(19) United States
(12) Patent Application Publication
(10) Pub. No.: US 2023/0312522 A1
(43) Pub. Date: Oct. 5, 2023

(54) CYP46A1 INHIBITORS AND METHODS OF USE THEREOF

(71) Applicant: Sage Therapeutics, Inc., Cambridge, MA (US)

(72) Inventors: Steven Mischke, Waltham, MA (US); Allen Taylor Hopper, Lexington, MA (US); Daniel La, Chestnut Hill, MA (US); Maria-Jesus Blanco-Pillado, Arlington, MA (US)

(21) Appl. No.: 18/201,885

(22) Filed: May 25, 2023

Related U.S. Application Data
(63) Continuation of application No. PCT/US2021/060844, filed on Nov. 24, 2021.
(60) Provisional application No. 63/118,291, filed on Nov. 25, 2020.

(51) Int. Cl. C07D 401/14; C07D 405/14; C07D 471/04; C07D 417/14

(52) U.S. Cl. CPC C07D 401/14; C07D 405/14; C07D 471/04; C07D 417/14

(57) ABSTRACT

Described herein are compounds that act as CYP46A1 inhibitors, compositions comprising these compounds, and methods for their use in treating neurodegenerative diseases and the like, or a pharmaceutically acceptable salt thereof.

The present disclosure relates to compounds represented by the formula

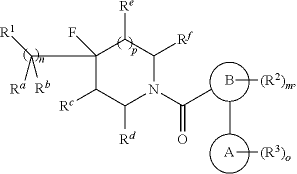

wherein each symbol is as defined in the specification, or a pharmaceutically acceptable salt thereof.

### RELATED APPLICATIONS

This Application is a continuation of International Patent Application No. PCT/US2021/060844, filed Nov. 24, 2021, which claims priority from and the benefit of U.S. Provisional Application No. 63/118,291, filed Nov. 25, 2020. The contents of each of the foregoing applications are incorporated herein by reference in their entireties.

### BACKGROUND

CYP46A1 is a gene expressed in the brain that encodes the enzyme cholesterol 24-hydroxylase (also known as CYP46A1 and CH24H), which converts cholesterol into 24S-hydroxycholesterol (24-HC), a positive allosteric modulator of N-methyl-D-aspartate (NMDA) receptors. Inhibition of 24-HC production in the brain by CYP46A1 inhibitors can negatively modulate glutamatergic over-activation in neurological diseases associated with NMDA hyperfunction, such as epilepsy and autism spectrum disorder (ASD); or diseases associated with elevated 24-HC levels, such as multiple sclerosis (MS). Findings have suggested that CYP46A1 inhibitors may also be promising therapeutics for neurodegenerative diseases such as Alzheimer's disease, Huntington's disease, Parkinson's disease, cerebral infarction, traumatic brain injury, glaucoma, and amyotrophic lateral sclerosis. The present disclosure provides compounds capable of modulating (e.g., inhibiting) CYP46A1.

### SUMMARY

Provided herein are compounds that act as CYP46A1 inhibitors and methods of use thereof. The compounds disclosed herein are useful as therapeutic agents for treating diseases associated with the inhibition of CYP46A1, for example, a neurodegenerative disease (e.g., Alzheimer's disease, mild cognitive impairment, Huntington's disease, Parkinson's disease, amyotrophic lateral sclerosis, traumatic brain injury, cerebral infarction, glaucoma, and multiple sclerosis), epilepsy, developmental and epileptic encephalopathies, psychiatric disorders (e.g., schizophrenia and autism spectrum disorder (ASD)), and spasms.

In a first aspect, the disclosure provides CYP46A1 inhibitors. In some embodiments, the disclosure provides a compound of Formula I:

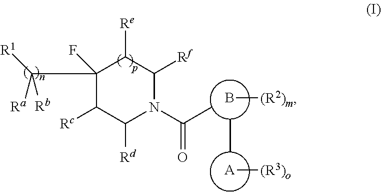

or a pharmaceutically acceptable salt thereof, wherein:
R¹ is selected from the group consisting of C₆-C₁₀ aryl, C₃-C₇ cycloalkyl, 3-7 membered heterocyclyl, and 5-10 membered heteroaryl, wherein R¹ is optionally substituted with one to four R⁴;
each of R^{a} and R^{b} is independently selected from the group consisting of H, halo, —CN, —OH, —NO₂, —N(R⁵)₂, C₁-C₆ alkyl, C₁-C₆ haloalkyl, C₁-C₆ alkoxy, and C₁-C₆ haloalkoxy; or R^{a} and R^{b} may form, together with the carbon to which they are attached, a C₃-C₇ cycloalkyl; or R^{a} and R^{b} taken together are oxo;
each of R^{c}, R^{d}, R^{e}, and R^{f} is independently selected from the group consisting of H, C₁-C₆ alkyl, C₁-C₆ haloalkyl, C₁-C₆ alkoxy, and C₁-C₆ haloalkoxy; or R^{c} and R^{e} may form, together with the carbons to which they are attached, a C₁-C₃ alkylene bridge; or R^{d} and R^{f} may form, together with the carbons to which they are attached, a C₁-C₃ alkylene bridge;
each R⁴ is independently selected from the group consisting of halo, —CN, —OH, —NO₂, —N(R⁵)₂, —S(O)₂R⁵, C₁-C₆ alkyl, C₁-C₆ haloalkyl, C₁-C₆ alkoxy, C₁-C₆ haloalkoxy, C₆-C₁₀ aryl, C₃-C₇ cycloalkyl, and 3-7 membered heterocyclyl;
each R⁵ is independently selected from H and C₁-C₆ alkyl;
each R² is independently selected from the group consisting of halo, —CN, —OH, —NO₂, —N(R⁵)₂, C₁-C₆ alkyl, C₃-C₇ cycloalkyl, C₁-C₆ haloalkyl, C₁-C₆ alkoxy, and C₁-C₆ haloalkoxy;
each R³ is independently selected from the group consisting of halo, —CN, —OH, —NO₂, —N(R⁵)₂, C₁-C₆ alkyl, C₁-C₆ haloalkyl, C₁-C₆ alkoxy, C₃-C₇ cycloalkyl, and C₁-C₆ haloalkoxy;
A is a 5-6 membered nitrogen-containing heteroaryl;
B is selected from C₆-C₁₀ aryl and 5-6 membered heteroaryl;
m is 0, 1, 2, or 3;
n is 0, 1, 2, 3, or 4;
is 0, 1, 2, or 3; and
p is 0, 1, or 2;
   provided that when n is 0, R¹ is not 4-cyanophenyl or 4-trifluomethylphenyl.

In some embodiments, the compound of Formula I is not:

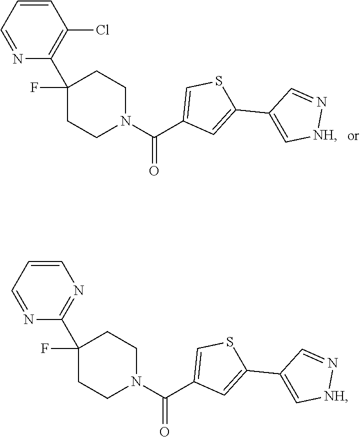 [SMILES: O=C(c1csc(-c2cn[nH]c2)c1)N1CCC(F)(c2ncccc2Cl)CC1 ; O=C(c1csc(-c2cn[nH]c2)c1)N1CCC(F)(c2ncccn2)CC1]

acceptable salt thereof.

In some embodiments, the compound of Formula I is not:

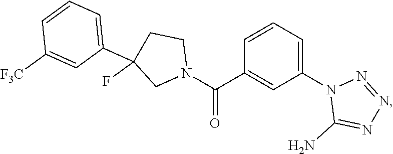 [SMILES: Nc1nnnn1-c1cccc(C(=O)N2CCC(F)(c3cccc(C(F)(F)F)c3)C2)c1]

or a pharmaceutically acceptable salt thereof.

In some embodiments, the compound of Formula I is a compound of Formula I-a-1:

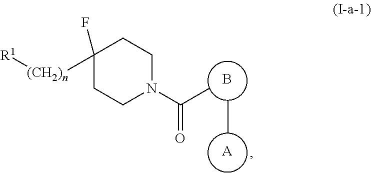

or a pharmaceutically acceptable salt thereof.

In some embodiments, R¹ is substituted C₆-C₁₀ aryl. In some embodiments, R¹ is unsubstituted C₆-C₁₀ aryl. In some embodiments, R¹ is

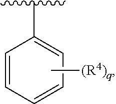

wherein each R⁴ is independently halo, —CN, —OH, —NO₂, —N(R⁵)₂, —S(O)₂R⁵, C₁-C₆ alkyl, C₁-C₆ haloalkyl, C₁-C₆ alkoxy, C₁-C₆ haloalkoxy, C₆-C₁₀ aryl, C₃-C₇ cycloalkyl, or 3-7 membered heterocyclyl; wherein each R⁵ is independently H or C₁-C₆ alkyl; and q is 0, 1, 2, or 3.

In some embodiments, R¹ is

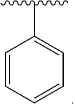 [SMILES: *c1ccccc1]

In some embodiments, R¹ is

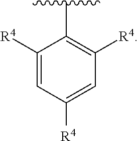

In some embodiments, R¹ is

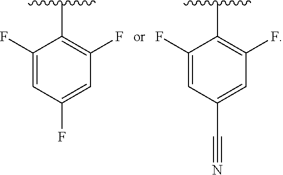

In some embodiments, R¹ is

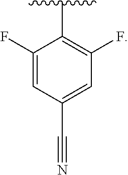 [SMILES: *c1c(F)cc(C#N)cc1F]

In some embodiments, R¹ is

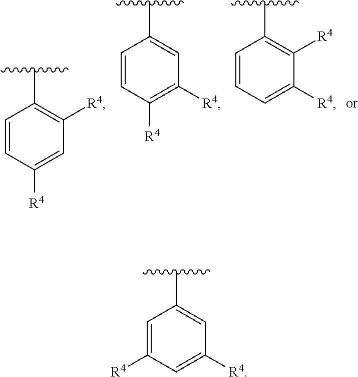

In some embodiments, R¹ is

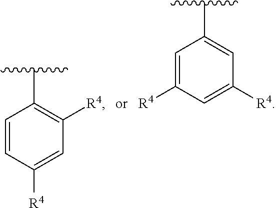

In some embodiments, R¹ is

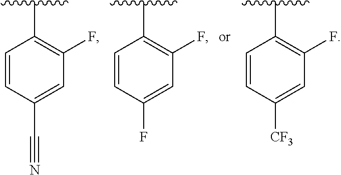

In some embodiments, R¹ is

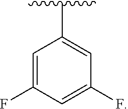 [SMILES: *c1cc(F)cc(F)c1]

In some embodiments, R¹ is

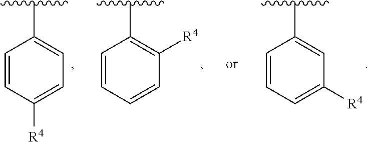

In some embodiments, R¹ is

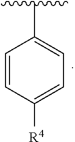

In some embodiments, R¹ is:

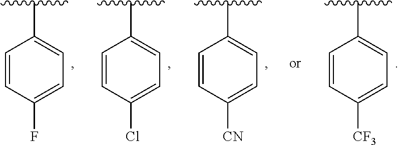

In some embodiments, R¹ is substituted 5-10 membered heteroaryl. In some embodiments, R¹ is unsubstituted 5-10 membered heteroaryl. In some embodiments, R¹ is pyrrolyl, furanyl, thiophenyl, imidazolyl, pyrazolyl, oxazolyl, isoxazolyl, thiazolyl, isothiazolyl, triazolyl, oxadiazolyl, thiadiazolyl, pyridyl, pyridazinyl, pyrimidinyl, pyrazinyl, triazinyl, tetrazinyl, tetrazolyl, azocinyl, dithiazinyl, or oxazinyl. In some embodiments, R¹ is pyridyl, pyridinyl, pyridazinyl, pyrimidinyl, or pyrazinyl. In some embodiments, R¹ is 2-pyridyl, 3-pyridyl, 4-pyridyl, 2-pyrimidinyl, or 4-pyrimidinyl.

In some embodiments, R¹ is

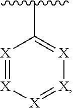

wherein each X is independently CH or N, wherein the H of CH is optionally substituted with one to four R⁴; wherein each R⁴ is independently halo, —CN, —OH, —NO₂, —N(R⁵)₂, C₁-C₆ alkyl, C₁-C₆ haloalkyl, C₁-C₆ alkoxy, C₁-C₆ haloalkoxy, C₆-C₁ aryl, C₃-C₇ cycloalkyl, or 3-7 membered heterocyclyl; and each R⁵ is independently H or C₁-C₆ alkyl. In some embodiments, R¹ is

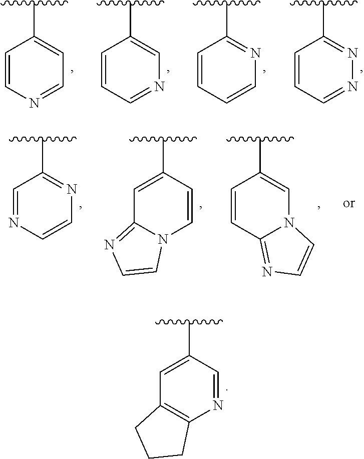

In some embodiments, R¹ is

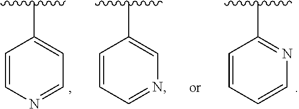

In some embodiments, R¹ is

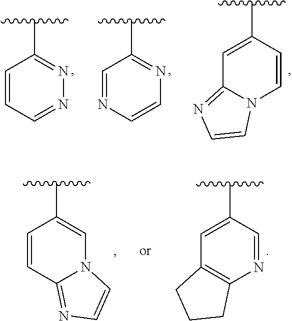

In some embodiments, R¹ is

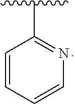 [SMILES: *c1ccccn1]

In some embodiments, R¹ is:

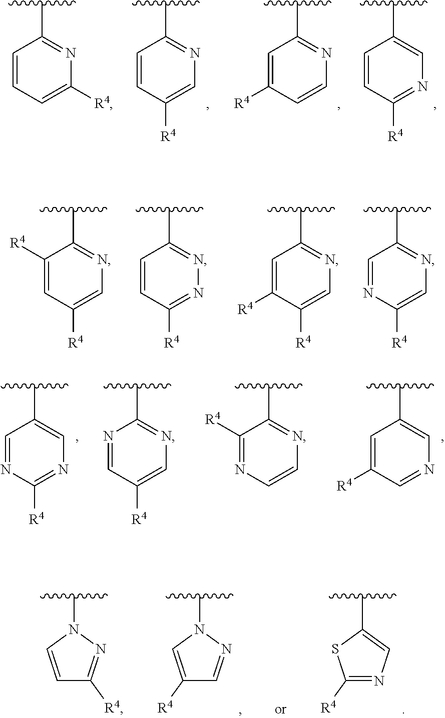

In some embodiments, R¹ is:

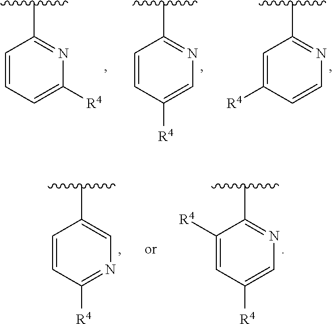

In some embodiments, R¹ is:

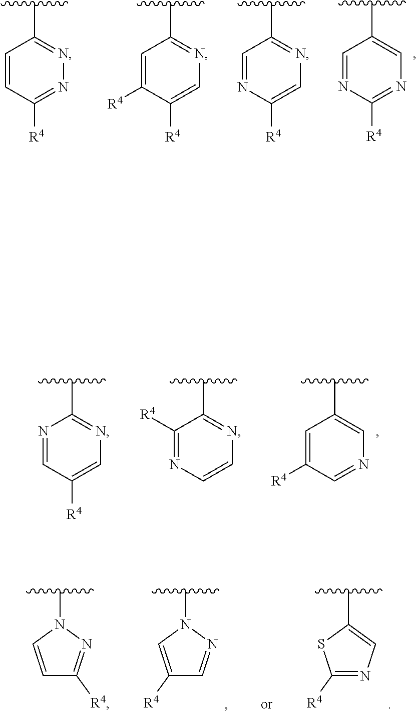

In some embodiments, R¹ is:

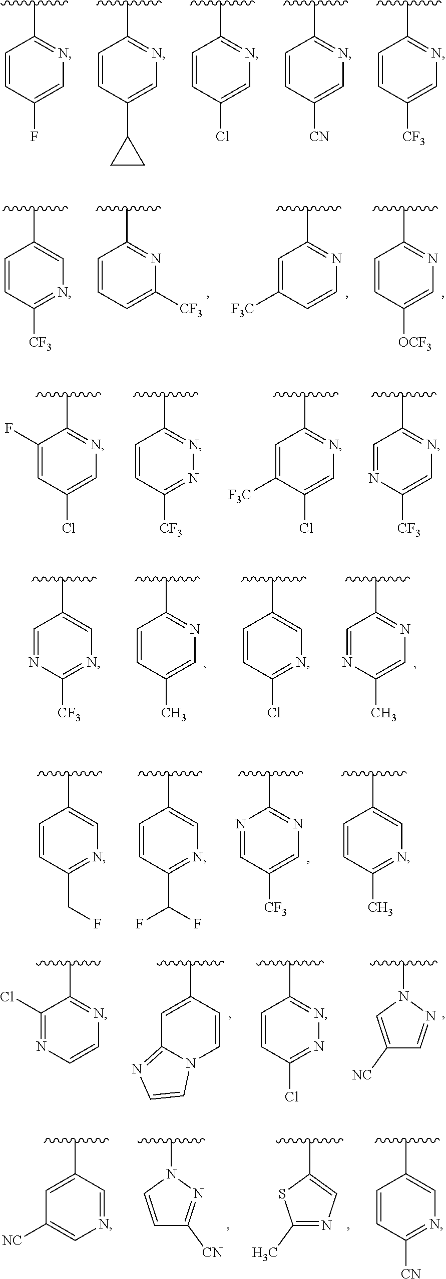

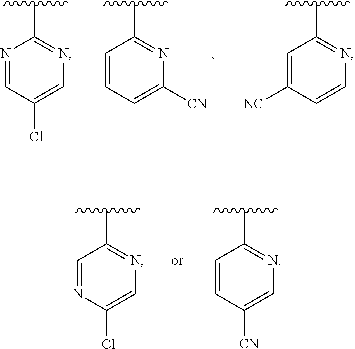

In some embodiments, R¹ is:

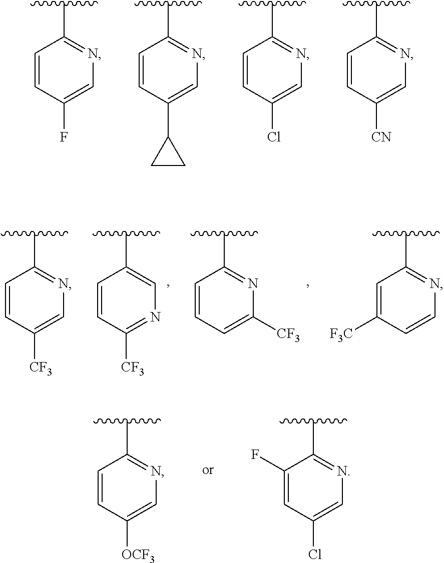

In some embodiments, R¹ is:

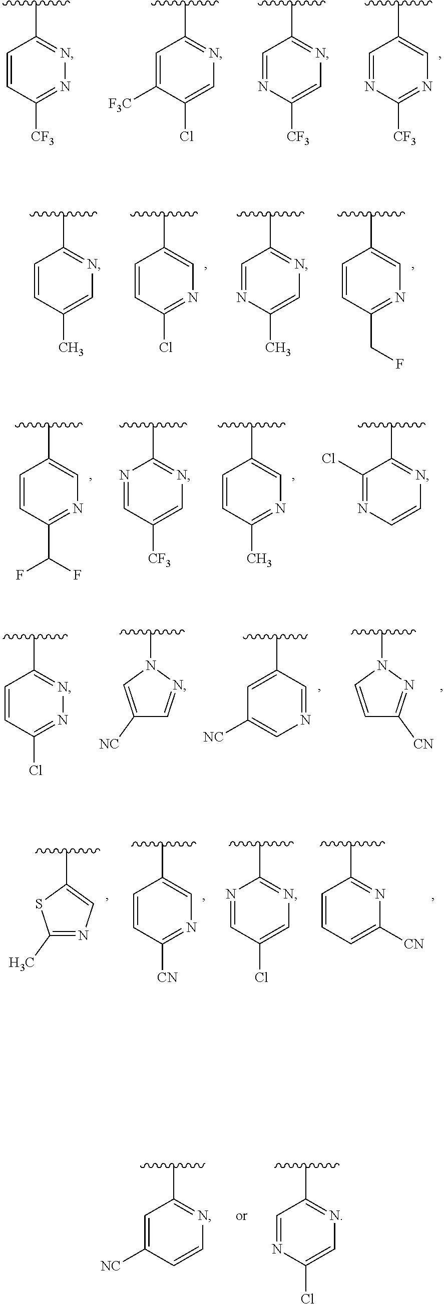

In some embodiments, R¹ is substituted 3-7 membered heterocyclyl. In some embodiments, R¹ is unsubstituted 3-7 membered heterocyclyl. In some embodiments, R¹ is tetrahydrofuran, tetrahydropyran, pyrrolidine, piperidine, piperazine, dioxolane, dioxane, thiomorpholine, or dithiane. In some embodiments, R¹ is tetrahydrofuran or tetrahydropyran. In some embodiments, R¹ is

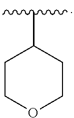 [SMILES: *C1CCOCC1]

In some embodiments, each R⁴ is independently halo, —CN, C₁-C₆ haloalkyl, and q is 0, 1, 2, or 3.

In some embodiments, each R⁴ is independently halo, —CN, substituted C₁-C₆ alkyl, substituted C₁-C₆ alkoxy, or substituted C₃-C₇ cycloalkyl. In some embodiments, each R⁴ is independently halo, —CN, unsubstituted C₁-C₆ alkyl, unsubstituted C₁-C₆ alkoxy, or unsubstituted C₃-C₇ cycloalkyl. In some embodiments, each R⁴ is independently halo, —CN, C₁-C₆ alkyl, C₁-C₆ haloalkyl, C₁-C₆ alkoxy, C₁-C₆ haloalkoxy, or C₃-C₇ cycloalkyl. In some embodiments, each R⁴ is independently halo, —CN, —CH₃, —CF₃, —CH₂F, —CHF₂, —OCH₃, —OCF₃, or cyclopropyl. In some embodiments, each R⁴ is independently halo, —CN, —CF₃, —OCF₃, or cyclopropyl. In some embodiments, each R⁴ is independently halo, —CN, —CH₃, —CF₃, —CH₂F, or —CHF₂. In some embodiments, each R⁴ is independently Cl, F, Br, or I. In some embodiments, each R⁴ is independently Cl, or F.

In some embodiments, n is 4. In some embodiments, n is 3. In some embodiments, n is 2. In some embodiments, n is 1. In some embodiments, n is 0.

In some embodiments, n is 1, and R^{a} is C₁-C₆ alkyl and R^{b} is H. In some embodiments, n is 1, and R^{a} is methyl and R^{b} is H. In some embodiments, n is 1, R^{a} is —OH, and R^{b} is H. In some embodiments, n is 1, and R^{a} and R^{b} are taken together to form an oxo. In some embodiments, n is 1, and R^{a} and R^{b} are both H.

In some embodiments, p is 2. In some embodiments, p is 1.

In some embodiments, p is 1, and R^{c}, R^{d}, R^{e}, and R^{f} are H. In some embodiments, p is 1, R^{c} is methyl, and R^{d}, R^{e}, and R^{f} are H. In some embodiments, p is 1, R^{c} and R^{e} are H, and R^{d} and R form together with the carbon to which they are attached, an C₁-C₃ alkylene bridge. In some embodiments, p is 1, R^{d} and R^{f} are H, and R^{c} and R^{e} form together with the carbon to which they are attached, an C₁-C₃ alkylene bridge.

In some embodiments, p is 0. In some embodiments, p is 0, and R^{c}, R^{d}, and R^{f} are H.

In some embodiments, B is

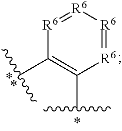

wherein each R⁶ is independently N or CR^{6a}, wherein R^{6a} is H or R²; and ** is the point of attachment to the carbonyl, and * is the point of attachment to A.

In some embodiments, up to two R⁶ may be N and the other occurrences of R⁶ are CH.

In some embodiments, B is:

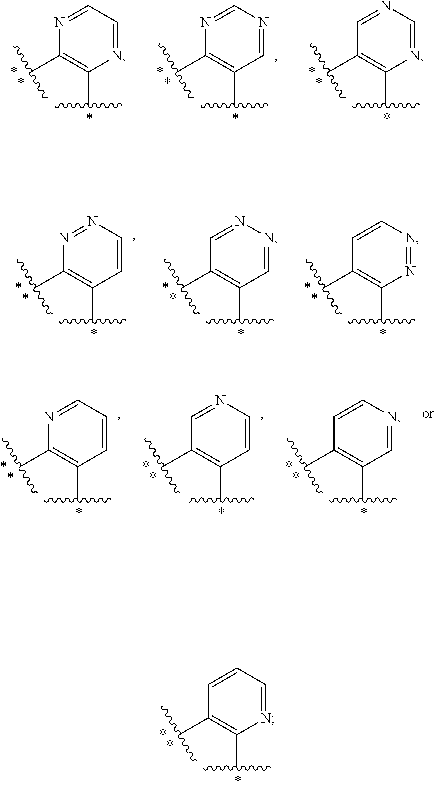

wherein ** is the point of attachment to a carbonyl, and * is the point of attachment to A. In some embodiments, B is

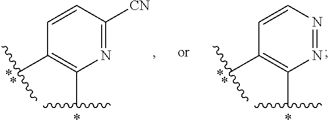 [SMILES: *c1ccc(C#N)nc1* ; *c1ccnnc1*]

wherein ** is the point of attachment to a carbonyl, and * is the point of attachment to A. In some embodiments, B is

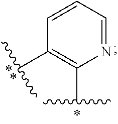 [SMILES: *c1cccnc1*]

wherein ** is the point of attachment to a carbonyl, and * is the point of attachment to A. In some embodiments, wherein B is

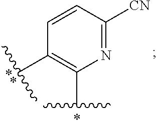 [SMILES: *c1ccc(C#N)nc1*]

wherein ** is the point of attachment to a carbonyl, and * is the point of attachment to A. In some embodiments, B is

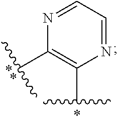 [SMILES: *c1nccnc1*]

wherein ** is the point of attachment to a carbonyl, and * is the point of attachment to A. In some embodiments, B is

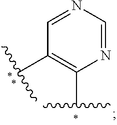 [SMILES: *c1cncnc1*]

wherein ** is the point of attachment to a carbonyl, and * is the point of attachment to A. In some embodiments, B is

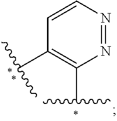 [SMILES: *c1ccnnc1*]

wherein ** is the point of attachment to a carbonyl, and * is the point of attachment to A. In some embodiments, B is

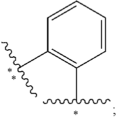 [SMILES: *c1ccccc1*]

wherein ** is the point of attachment to a carbonyl, and * is the point of attachment to A.

In some embodiments, m is 3. In some embodiments, m is 2. In some embodiments, m is 1. In some embodiments, m is 0.

In some embodiments, A is pyridinyl, pyrrolyl, imidazolyl, pyrazolyl, pyridazinyl, pyrimidinyl, pyrazinyl, triazolyl, triazinyl, tetrazinyl, tetrazolyl, oxazolyl, isoxazolyl, or thiozolyl. In some embodiments, A is pyridinyl oxazolyl, imidazolyl, triazolyl, pyridinyl, pyridazinyl, pyrimidinyl, or triazinyl.

In some embodiments, A is

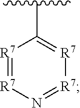

wherein each R⁷ is independently N or CH, wherein up to two R⁷ may be N and the other occurrences of R⁷ are CH, wherein the hydrogen of CH may be substituted with R³. In some embodiments A is:

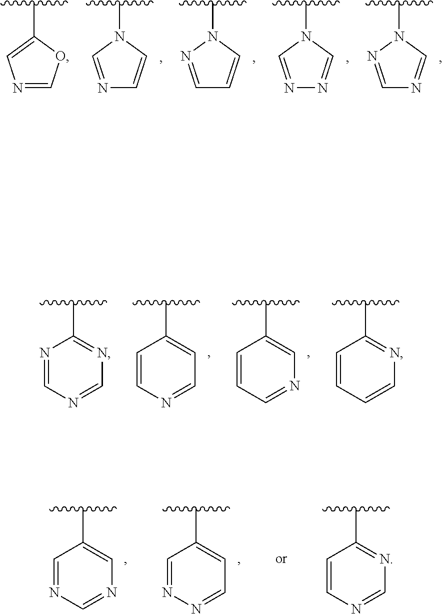

In some embodiments, A is:

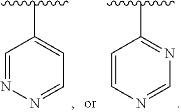 [SMILES: *c1ccnnc1 ; *c1ccncn1]

In some embodiments, the compound is selected from the group consisting of Compounds 1-98, or a pharmaceutically acceptable salt thereof. In some embodiments, the compound is selected from the group consisting of Compounds 1, 4-7, 9, 11, 13-15, 18-20, 22-24, 26, 28-29, 31-35, 38-39, 43, 46-47, 51-52, 54-55, and 57. In some embodiments, the compound is selected from the group consisting of Compounds 10, 12, 16-17, 21, 25, 27, 30, 36-37, 40-42, 44, 48-50 and 53.

In a second aspect, the disclosure provides a pharmaceutical composition comprising a compound of the disclosure, or a pharmaceutically acceptable salt thereof, and a pharmaceutically acceptable carrier.

In a third aspect, the disclosure provides a method for treating or preventing a disease or disorder involving the inhibition of CYP46A1 in a subject in need thereof, comprising administering to the subject therapeutically effective amount of a compound of the disclosure, or a pharmaceutically acceptable salt thereof, or a pharmaceutical composition of the disclosure, wherein the disease or disorder involving the inhibition of CYP46A1 is selected from the group consisting of a neurodegenerative disorder, epilepsy, developmental and epileptic encephalopathies, psychiatric disorders, and spasms.

In some embodiments, the disease or disorder involving the inhibition of CYP46A1 is a neurodegenerative disorder. In some embodiments, the neurodegenerative disorder is selected from the group consisting of Alzheimer's disease, mild cognitive impairment, Huntington's disease, Parkinson's disease, amyotrophic lateral sclerosis, traumatic brain injury, cerebral infarction, glaucoma, and multiple sclerosis.

In some embodiments, the disease or disorder involving the inhibition of CYP46A1 is epilepsy.

In some embodiments, the disease or disorder involving the inhibition of CYP46A1 is developmental and epileptic encephalopathies.

In some embodiments, the disease or disorder involving the inhibition of CYP46A1 is a psychiatric disorder. In some embodiments, the psychiatric disorder is selected from the group consisting of schizophrenia, autism spectrum disorder, delusional disorder, schizoaffective disorder, and depression.

In some embodiments, the disease or disorder involving the inhibition of CYP46A1 is spasms.

In a fourth aspect, the disclosure provides a compound or pharmaceutically acceptable salt thereof of the disclosure, or a pharmaceutically acceptable salt thereof, or a pharmaceutical composition of the disclosure, for use in treating or preventing a disease or disorder involving the inhibition of CYP46A1 in a subject. In some embodiments, the disease or disorder involving the inhibition of CYP46A1 is selected from the group consisting of a neurodegenerative disorder, epilepsy, developmental and epileptic encephalopathies, psychiatric disorders, and spasms.

In some embodiments, the compound or pharmaceutically acceptable salt thereof or pharmaceutical composition is for use in treating or preventing a neurodegenerative disorder. In some embodiments, the neurodegenerative disorder is selected from the group consisting of Alzheimer's disease, mild cognitive impairment, Huntington's disease, Parkinson's disease, amyotrophic lateral sclerosis, traumatic brain injury, cerebral infarction, glaucoma, and multiple sclerosis.

In some embodiments, the compound or pharmaceutically acceptable salt thereof or pharmaceutical composition is for use in treating or preventing epilepsy.

In some embodiments, the compound or pharmaceutically acceptable salt thereof or pharmaceutical composition is for use in treating or preventing developmental and epileptic encephalopathies.

In some embodiments, the compound or pharmaceutically acceptable salt thereof or pharmaceutical composition is for use in treating or preventing a psychiatric disorder. In some embodiments, the psychiatric disorder is selected from the group consisting of schizophrenia, autism spectrum disorder, delusional disorder, schizoaffective disorder, and depression.

In some embodiments, the compound or pharmaceutically acceptable salt thereof or pharmaceutical composition is for use in treating or preventing spasms.

In a fifth aspect, the disclosure provides a use of a compound or pharmaceutically acceptable salt thereof of the disclosure in the manufacture of a medicament for treating or preventing a disease or disorder involving the inhibition of CYP46A1 in a subject. In some embodiments, the disease or disorder involving the inhibition of CYP46A1 is selected from the group consisting of a neurodegenerative disorder, epilepsy, developmental and epileptic encephalopathies, psychiatric disorders, and spasms.

In some embodiments, the disease or disorder involving the inhibition of CYP46A1 is a neurodegenerative disorder. In some embodiments, the neurodegenerative disorder is selected from the group consisting of Alzheimer's disease, mild cognitive impairment, Huntington's disease, Parkinson's disease, amyotrophic lateral sclerosis, traumatic brain injury, cerebral infarction, glaucoma, and multiple sclerosis.

In some embodiments, the disease or disorder involving the inhibition of CYP46A1 is epilepsy.

In some embodiments, the disease or disorder involving the inhibition of CYP46A1 is developmental and epileptic encephalopathies.

In some embodiments, the disease or disorder involving the inhibition of CYP46A1 is a psychiatric disorder. In some embodiments, the psychiatric disorder is selected from the group consisting of schizophrenia, autism spectrum disorder, delusional disorder, schizoaffective disorder, and depression.

In some embodiments, the disease or disorder involving the inhibition of CYP46A1 is spasms.

### DETAILED DESCRIPTION

The present disclosure provides compounds that are CYP46A1 inhibitors. The compounds of the disclosure are useful as therapeutic agents for treating neurodegenerative disease (e.g. Alzheimer's disease, mild cognitive impairment, Huntington's disease, Parkinson's disease, amyotrophic lateral sclerosis, traumatic brain injury, cerebral infarction, glaucoma, and multiple sclerosis), epilepsy, developmental and epileptic encephalopathies, psychiatric disorders (e.g. schizophrenia and autism spectrum disorder), and spasms.

### General Definitions

The term “herein” means the entire application.

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments of the disclosure, unless explicitly disclaimed or improper. Combination of embodiments are not limited to those specific combinations claimed via the multiple dependent claims. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range and including the endpoints, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

All of the publications, patents and published patent applications referred to in this application are specifically incorporated by reference herein. In case of conflict, the present specification, including its specific definitions, will control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers, e.g., stereoisomers, can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound. As used herein, the term “diastereomeric purity” refers to the amount of a compound having the depicted absolute stereochemistry, expressed as a percentage of the total amount of the depicted compound and its diastereomers. The term “diastereomerically pure” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the diastereomer. Methods for determining diastereomeric and enantiomeric purity are well-known in the art. Diastereomeric purity can be determined by any analytical method capable of quantitatively distinguishing between a compound and its diastereomers, such as high performance liquid chromatography (HPLC).

In the compositions provided herein, an enantiomerically pure compound can be present with other active or inactive ingredients. For example, a pharmaceutical composition comprising enantiomerically pure R-position/center/carbon compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

As used herein, the term “diastereomeric purity” refers to the amount of a compound having the depicted absolute stereochemistry, expressed as a percentage of the total amount of the depicted compound and its diastereomers. The term “diastereomerically pure” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the diastereomer. Methods for determining diastereomeric and enantiomeric purity are well-known in the art. Diastereomeric purity can be determined by any analytical method capable of quantitatively distinguishing between a compound and its diastereomers, such as high performance liquid chromatography (HPLC).

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₄ alkyl.

The following terms are intended to have the meanings presented therewith below and are useful in understanding the description and intended scope of the present disclosure. It should also be understood that when described herein any of the moieties defined herein may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

“Alkyl” refers to a radical of a straight-chain or branched saturated hydrocarbon group having from 1 to 20 carbon atoms (“C₁₋₂₀ alkyl”). In some embodiments, an alkyl group has 1 to 6 carbon atoms (“C₁₋₄ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 4 substituents, 1 to 3 substituents, or 1 substituent. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

“Alkylene” refers to a bivalent saturated hydrocarbon. Alkylenes can be represented by —(CH₂)ₙ—, —(CH₂)—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, or —(CH₂)₁₀—. In some embodiments, alkylenes can be an indicated number of carbon atoms, for example, C₁-C₄ alkylene, C₁-C₃ alkylene, or C₁-C₂ alkylene. Unless otherwise specified, each instance of an alkylene group is independently optionally substituted, i.e., unsubstituted (an “unsubstituted alkylene”) or substituted (a “substituted alkylene”) with one or more substituents (for instance from 1 to 4 substituents, 1 to 3 substituents, or 1 substituent) which may be halo, —NO₂, —OH, C₁-C₆ alkoxy, C₁-C₆ alkyl, or C₁-C₆ cycloalkyl. Alkylene abbreviations include —(CH(CH₃))—, —(CH(CH₂CH₃))—, —(CH(CH₂CH₂CH₃))—, —(CH(CH₂CH₂CH₂CH₃))—, —(CH₂CH(CH₂CH₂CH₂CH₃))—, —(CH₂CH₂CH(CH₂CH₂CH₂CH₃))—, —(CH(CH₃)CH₂)—, —(CH(CH₃)CH₂CH₂)—, —(CH(CH₃)CH₂CH₂CH₂)—, —(CH₂CH(CH₃)CH₂)—, —(CH₂CH(CH₃)CH₂CH₂)—, and —(CH₂CH₂CH(CH₃)CH₂CH₂)—.

“Aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). Aryl” also includes ring systems wherein the aryl ring, as defined herein, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from benzene. Particularly aryl groups include phenyl, and indenyl. Unless otherwise specified, each instance of an aryl group is independently optionally substituted, i.e., unsubstituted (an “unsubstituted aryl”) or substituted (a “substituted aryl”) with one or more substituents.

“Heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system.

In some embodiments, a heteroaryl group is a 1-4 ring heteroatoms provided in the aromatic ring system, wherein each heteroatom is independently selected from nitrogen, oxygen, and sulfur. In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur. In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively.

Examples of representative heteroaryls include the following:

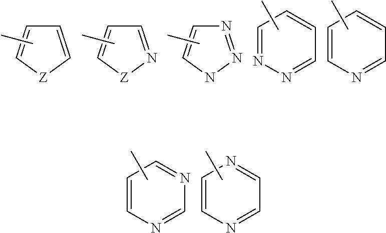

wherein each Z is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

“Alkylene bridge” refers to a straight or branched divalent hydrocarbon bridge, linking two different carbons of the same ring structure. The alkylene bridge may link any two carbons within the ring structure. In some embodiments, alkylene bridges can be an indicated number of carbon atoms, for example, C₁-C₆ alkylene bridge, C₁-C₅ alkylene bridge, C₁-C₄ alkylene bridge, C₁-C₃ alkylene bridge, or C₁-C₂ alkylene bridge. Unless otherwise specified, each instance of an alkylene bridge is independently optionally substituted, i.e., unsubstituted (an “unsubstituted alkylene bridge”) or substituted (a “substituted alkylene bridge”) with one or more substituents (for instance from 1 to 4 substituents, 1 to 3 substituents, or 1 substituent) which may be halo, —NO₂, —OH, C₁-C₆ alkoxy, C₁-C₆ alkyl, or C₁-C₆ cycloalkyl. Examples of alkylene bridge include, but are not limited to, methylene, ethylene, propylene, tetramethylene, and n-butylene.

“Nitrogen-containing heteroaryl” refers to a monocyclic aromatic heterocyclic group containing at least one nitrogen atom. Exemplary nitrogen-containing heteroaryl groups include, but without limitation, pyrrolyl, thiazolyl, isoxazolyl, pyrazinyl, imidazolyl, oxazolyl, pyridyl (e.g. 2-pyridyl, 3-pyridyl, 4-pyridyl), pyrimidinyl (e.g. 2-pyrimidinyl, 4-pyrimidinyl), pyridazinyl, triazolyl, triazinyl, tetrazolyl, azepinyl, azocinyl, dithiazinyl, and oxazinyl.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₄ carbocyclyl”). In some embodiments, a carbocyclyl group has 3 to 6 ring carbon atoms (“C₃₋₄ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₄ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Nitrogen-containing heterocyclyl” group refers to a non-aromatic heterocyclic group containing at least one nitrogen atom. Exemplary nitrogen-containing heterocyclyl groups include, but are not limited to, morpholinyl, piperidinyl (e.g. 2-piperidinyl, 3-piperidinyl and 4-piperidinyl), pyrrolidinyl (e.g. 2-pyrrolidinyl and 3-pyrrolidinyl), azetidinyl, pyrrolidonyl, imidazolinyl, imidazolidinonyl, 2-pyrazolinyl, pyrazolidinyl, piperazinyl, (e.g., N-alkyl piperazines such as N-methyl piperazine).

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₅ alkyl, C₃-C₅ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₅ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

Haloalkoxy” refers to a haloalkyl group as defined herein attached through an oxygen bridge (oxygen of an alcohol radical).

“Halo” or “halogen” refers to fluoro (F), chloro (Cl), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl, difluoromethyl, fluoromethyl, chloromethyl, dichloromethyl, dibromoethyl, tribromomethyl, tetrafluoroethyl, and the like.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc}), —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa}), —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc}), —NR^{bb}P(═O)(NR^{bb})₂, —P(R^{cc}), —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, —NNR^{bb}C(═O)R^{aa}, ═NNR^{bb}C(═O)OR^{aa}, —NNR^{bb}S(═O)₂R^{aa}, ═NR^{bb}, or ═NOR^{cc};
wherein each instance of R^{a} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})—CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc}), —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ee})₂, —OC(═O)N(R^{f})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ee})₂, —NR^{ee}C(═NR^{ee})N(R^{ee})₂, —NR^{ff}SO₂R^{ee}, —SO₂N(R^{f})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₄ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃+X⁻, —NH(C₁₋₆ alkyl)₂⁺X⁻, —NH₂(C₁₋₆ alkyl)⁺X⁻, —NH₃+X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁_alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa} C(═O)N(R^{bb})₂, —C(═NR^{bb})R^{aa}, —C(═NR^{bb})OR—, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa}), —P(═O)(OR^{cc}), —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), 2-methoxyethoxymethyl (MEM), benzyl (Bn), triisopropylsilyl (TIPS), t-butyldimethylsilyl (TBDMS), t-butylmethoxyphenylsilyl (TBMPS), methanesulfonate (mesylate), and tosylate (Ts).

In certain embodiments, the substituent present on a nitrogen atom is an amino protecting group (also referred to herein as a nitrogen protecting group). Amino protecting groups include, but are not limited to, —OH, —OR^{aa}, —N(R^{cc}), —C(═O)R^{aa}, —C(═O)OR^{aa}, C(═O)N(R^{cc}), —S(═O)₂R^{aa}, —C(═NR^{cc})R^{aa}, —C(═NR^{cc})OR^{aa}, —C(═NR^{cc})N(R^{cc}), —SO₂N(R^{cc})₂, —SO₂R^{cc}, —SO₂OR^{cc}, —SOR^{aa}, —C(═S)N(R^{cc}), —C(═O)SR^{cc}, —C(═S)SR^{cc}, C₁₋₁₀ alkyl, C₂₋₁₀ alkenyl, C₂₋₁₀ alkynyl, C₃₋₁₀ carbocyclyl, 3-14-membered heterocyclyl, C₆₋₁₄ aryl, and 5-14-membered heteroaryl groups, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein. Amino protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary amino protecting groups include, but are not limited to amide groups (e.g., —C(═O)R^{aa}), which include, but are not limited to, formamide and acetamide; carbamate groups (e.g., —C(═O)OR^{aa}), which include, but are not limited to, 9-fluorenylmethyl carbamate (Fmoc), t-butyl carbamate (BOC), and benzyl carbamate (Cbz); sulfonamide groups (e.g., —S(═O)₂R^{aa}) which include, but are not limited to, p-toluenesulfonamide (Ts), methanesulfonamide (Ms), and N-[2-(trimethylsilyl)ethoxy]methylamine (SEM).

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound disclosed herein that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

“Pharmaceutically acceptable carrier” refers to compositions, carriers, diluents, and reagents which are pharmaceutically acceptable materials that are capable of administration to or upon a subject. A pharmaceutically acceptable carrier can be involved with carrying or transporting the subject agents from one organ, or portion of the body, to another organ, or portion of the body. The carrier can be in the form of a solid, semi-solid or liquid diluent, cream or a capsule. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof.

A “subject” to which administration is contemplated includes, but is not limited to, human subject (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

Disease, disorder, and condition are used interchangeably herein.

As used herein, the term “treat,” “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation, amelioration, reduction of the severity, or slowing the progression, of one or more symptoms or conditions associated with a condition, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Exemplary beneficial clinical results are described herein.

As used herein, and unless otherwise specified, the term “prophylactic” contemplates an action that occurs before a subject begins to suffer from the specified disease, disorder, or condition.

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

The terms “pharmaceutically effective amount,” “therapeutically effective amount,” or “therapeutically effective dose” refer to an amount sufficient to treat a disease in a patient, e.g., effecting a beneficial and/or desirable alteration in the health of a patient suffering from a disease, treatment, healing, inhibition or amelioration of a physiological response or condition, delaying or minimizing one or more symptoms associated with the disease, disorder or condition etc. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of disease, the therapeutics or combination of therapeutics selected for administration, and the mode of administration. The skilled worker can readily determine the effective amount for a given situation by routine experimentation. The terms “pharmaceutically effective amount,” “therapeutically effective amount,” or “therapeutically effective dose” also refer to the amount required to improve the clinical symptoms of a patient. A therapeutically effective amount of a compound also refers to an amount of the therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, and unless otherwise specified, “pharmacokinetics” can be defined as the study of bodily absorption, distribution, metabolism, and excretion of drugs. “Pharmacokinetics” can also be defined as the characteristic interactions of a drug and a body in terms of its absorption, distribution, metabolism, and excretion; or a branch of pharmacology concerned with the way drugs are taken into, move around, and are eliminated from, a body.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. When a method is part of a therapeutic regimen involving more than one agent or treatment modality, the disclosure contemplates that the agents may be administered at the same or differing times and via the same or differing routes of administration. Appropriate methods of administering a substance, a compound or an agent to a subject will also depend, for example, on the age of the subject, whether the subject is active or inactive at the time of administering, whether the subject is cognitively impaired at the time of administering, the extent of the impairment, and the chemical and biological properties of the compound or agent (e.g. solubility, digestibility, bioavailability, stability and toxicity).

### Compounds

In one aspect the present disclosure provides compounds of Formula I. In some embodiments, the compounds inhibit CYP46A1 and can be used in the treatment of neurodegenerative diseases, epilepsy, developmental and epileptic encephalopathies, psychiatric disorders, and spasm. For example, in an aspect, described herein are compounds of Formula I:

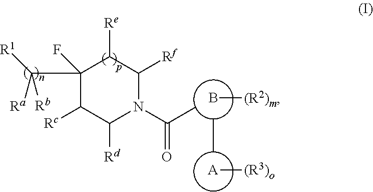

or a pharmaceutically acceptable salt thereof, wherein:
R¹ is selected from the group consisting of C₆-C₁₀ aryl, C₃-C₇ cycloalkyl, 3-7 membered heterocyclyl, and 5-10 membered heteroaryl, wherein R¹ is optionally substituted with one to four R⁴;
each of R^{a} and R^{b} is independently selected from the group consisting of H, halo, —CN, —OH, —NO₂, —N(R⁵)₂, C₁-C₆ alkyl, C₁-C₆ haloalkyl, C₁-C₆ alkoxy, and C₁-C₆ haloalkoxy; or R^{a} and R^{b} may form, together with the carbon to which they are attached, a C₃-C₇ cycloalkyl; or R^{a} and R^{b} taken together are oxo;
each of R^{c}, R^{d}, R^{e}, and R^{f} is independently selected from the group consisting of H, C₁-C₆ alkyl, C₁-C₆ haloalkyl, C₁-C₆ alkoxy, and C₁-C₆ haloalkoxy; or R^{c} and R^{e} may form, together with the carbons to which they are attached, a C₁-C₃ alkylene bridge; or R^{d} and R^{f} may form, together with the carbons to which they are attached, a C₁-C₃ alkylene bridge;
each R⁴ is independently selected from the group consisting of halo, —CN, —OH, —NO₂, —N(R⁵)₂, —S(O)₂R⁵, C₁-C₆ alkyl, C₁-C₆ haloalkyl, C₁-C₆ alkoxy, C₁-C₆ haloalkoxy, C₆-C₁₀ aryl, C₃-C₇ cycloalkyl, and 3-7 membered heterocyclyl;
each R⁵ is independently selected from H and C₁-C₆ alkyl;
each R² is independently selected from the group consisting of halo, —CN, —OH, —NO₂, —N(R⁵)₂, C₁-C₆ alkyl, C₃-C₇ cycloalkyl, C₁-C₆ haloalkyl, C₁-C₆ alkoxy, and C₁-C₆ haloalkoxy;
each R³ is independently selected from the group consisting of halo, —CN, —OH, —NO₂, —N(R⁵)₂, C₁-C₆ alkyl, C₁-C₆ haloalkyl, C₁-C₆ alkoxy, C₃-C₇ cycloalkyl, and C₁-C₆ haloalkoxy;
A is a 5-6 membered nitrogen-containing heteroaryl;
B is selected from C₆-C₁₀ aryl and 5-6 membered heteroaryl;
m is 0, 1, 2, or 3;
n is 0, 1, 2, 3, or 4;
is 0, 1, 2, or 3; and
p is 0, 1, or 2;
provided that when n is 0, R¹ is not 4-cyanophenyl or 4-trifluomethylphenyl.

In some embodiments, the compound of Formula I is not:

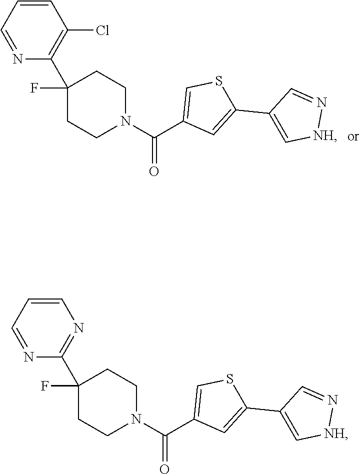 [SMILES: O=C(c1csc(-c2cn[nH]c2)c1)N1CCC(F)(c2ncccc2Cl)CC1 ; O=C(c1csc(-c2cn[nH]c2)c1)N1CCC(F)(c2ncccn2)CC1]

or a pharmaceutically acceptable salt thereof.

In some embodiments, the compound of Formula I is not:

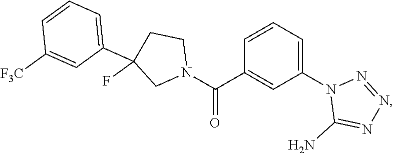 [SMILES: Nc1nnnn1-c1cccc(C(=O)N2CCC(F)(c3cccc(C(F)(F)F)c3)C2)c1]

or a pharmaceutically acceptable salt thereof.

In some embodiments, the compound of Formula I is a compound of Formula I:

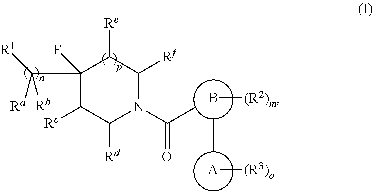

or a pharmaceutically acceptable salt thereof, wherein:
R¹ is selected from the group consisting of C₆-C₁₀ aryl, C₃-C₇ cycloalkyl, 3-7 membered heterocyclyl, and 5-10 membered heteroaryl, wherein R¹ is optionally substituted with one to four R⁴;
each of R^{a} and R^{b} is independently selected from the group consisting of H, halo, —CN, —OH, —NO₂, —N(R⁵)₂, C₁-C₆ alkyl, C₁-C₆ haloalkyl, C₁-C₆ alkoxy, and C₁-C₆ haloalkoxy; or R^{a} and R^{b} may form, together with the carbon to which they are attached, a C₃-C₇ cycloalkyl; or R^{a} and R^{b} taken together are oxo;
each of R^{c}, R^{d}, R^{c}, and R^{f} is independently selected from the group consisting of H, C₁-C₆ alkyl, C₁-C₆ haloalkyl, C₁-C₆ alkoxy, and C₁-C₆ haloalkoxy; or R^{c} and R^{e} may form, together with the carbons to which they are attached, a C₁-C₃ alkylene bridge; or R^{d} and R^{e} may form, together with the carbons to which they are attached, a C₁-C₃ alkylene bridge;
each R⁴ is independently selected from the group consisting of halo, —CN, —OH, —NO₂, —N(R⁵)₂, —S(O)₂R⁵, C₁-C₆ alkyl, C₁-C₆ haloalkyl, C₁-C₆ alkoxy, C₁-C₆ haloalkoxy, C₆-C₁₀ aryl, C₃-C₇ cycloalkyl, and 3-7 membered heterocyclyl;
each R⁵ is independently selected from H and C₁-C₆ alkyl;
each R² is independently selected from the group consisting of halo, —CN, —OH, —NO₂, —N(R⁵)₂, C₁-C₆ alkyl, C₃-C₇ cycloalkyl, C₁-C₆ haloalkyl, C₁-C₆ alkoxy, and C₁-C₆ haloalkoxy;
each R³ is independently selected from the group consisting of halo, —CN, —OH, —NO₂, —N(R⁵)₂, C₁-C₆ alkyl, C₁-C₆ haloalkyl, C₁-C₆ alkoxy, C₃-C₇ cycloalkyl, and C₁-C₆ haloalkoxy;
A is a 5-6 membered nitrogen-containing heteroaryl;
B is selected from C₆-C₁₀ aryl and 5-6 membered heteroaryl;
m is 0, 1, 2, or 3;
n is 0, 1, 2, 3, or 4;
is 0, 1, 2, or 3; and
p is 0, 1, or 2;
   provided that when n is 0, R¹ is not 4-cyanophenyl or 4-trifluomethylphenyl and further provided that the compound of Formula I is not:

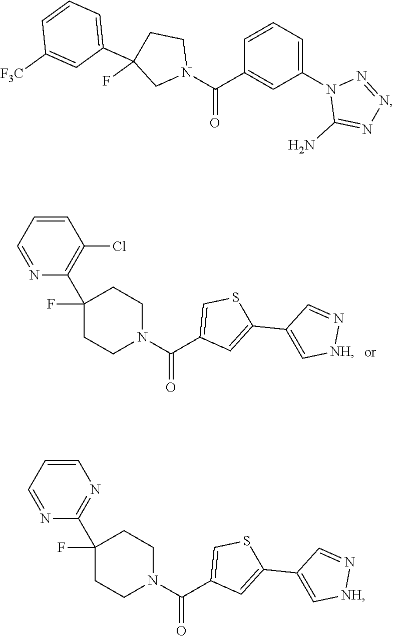 [SMILES: Nc1nnnn1-c1cccc(C(=O)N2CCC(F)(c3cccc(C(F)(F)F)c3)C2)c1 ; O=C(c1csc(-c2cn[nH]c2)c1)N1CCC(F)(c2ncccc2Cl)CC1 ; O=C(c1csc(-c2cn[nH]c2)c1)N1CCC(F)(c2ncccn2)CC1]

or a pharmaceutically acceptable salt thereof.

In some embodiments, the compound of Formula I is a compound of Formula I-a:

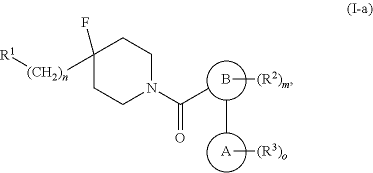

or a pharmaceutically acceptable salt thereof; wherein A, B, R¹, R², R³, m, n and o are as defined herein.

In some embodiments, the compound of Formula I-a is a compound of Formula I-a-1

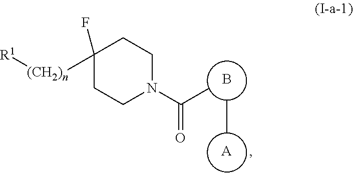

or a pharmaceutically acceptable salt thereof, wherein A, B, R¹ and n are as defined herein.

In some embodiments, the compound of Formula I-a is a compound of Formula I-a-2

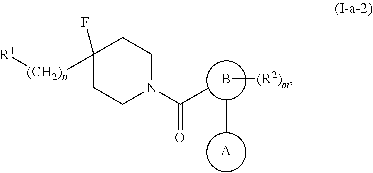

or a pharmaceutically acceptable salt thereof; wherein A, B, R¹, R², m, and n are as defined herein.

In some embodiments, the compound of Formula I-a is a compound of Formula I-a-3

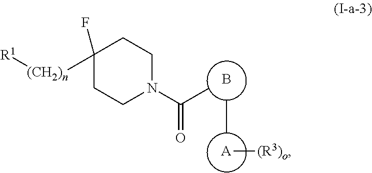

or a pharmaceutically acceptable salt thereof, wherein A, B, R¹, R³, n and o are as defined herein.

In some embodiments, the compound of Formula I is a compound of Formula I-b:

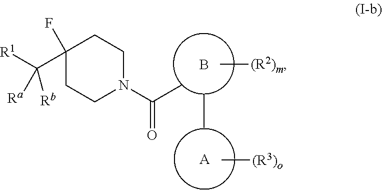

or a pharmaceutically acceptable salt thereof, wherein A, B, R¹, R², R³, R^{a}, R^{b}, m, and o are as defined herein.

In some embodiments, the compound of Formula I-b is a compound of Formula I-b-1:

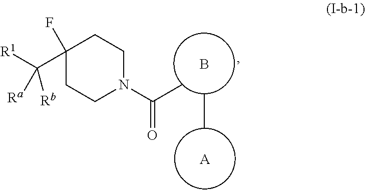

or a pharmaceutically acceptable salt thereof; wherein A, B, R¹, R^{a}, and R^{b} are as defined herein.

In some embodiments, the compound of Formula I-b is a compound of Formula I-b-2:

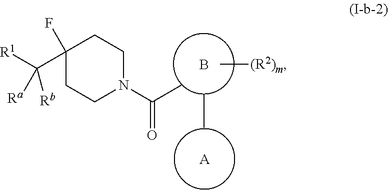

or a pharmaceutically acceptable salt thereof; wherein A, B, R¹, R², R^{a}, R^{b}, and m are as defined herein.

In some embodiments, the compound of Formula I-b is a compound of Formula I-b-3:

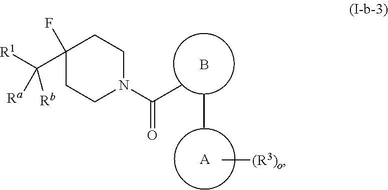

or a pharmaceutically acceptable salt thereof, wherein A, B, R¹, R³, R^{a}, R^{b}, and o are as defined herein.

In some embodiments, the compound of Formula I is a compound of Formula I-c:

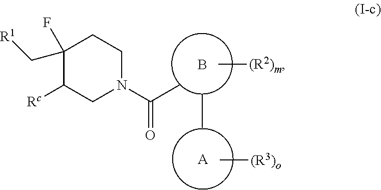

or a pharmaceutically acceptable salt thereof; wherein A, B, R¹, R², R³, R^{c}, m, and o are as defined herein.

In some embodiments, the compound of Formula I-c is a compound of Formula I-c-1:

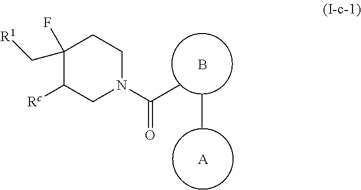

or a pharmaceutically acceptable salt thereof, wherein A, B, R¹, and R^{c} are as defined herein.

In some embodiments, the compound of Formula I-c is a compound of Formula I-c-2:

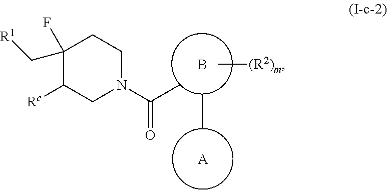

or a pharmaceutically acceptable salt thereof; wherein A, B, R¹, R², R^{c}, and m are as defined herein.

In some embodiments, the compound of Formula I-c is a compound of Formula I-c-3:

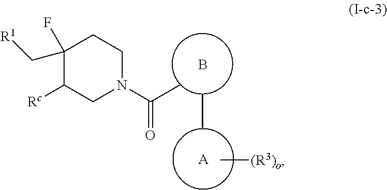

or a pharmaceutically acceptable salt thereof, wherein A, B, R¹, R³, R^{c}, and o are as defined herein.

In some embodiments, the compound of Formula I is a compound of Formula I-d:

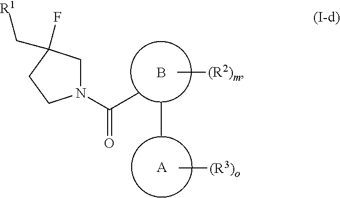

or a pharmaceutically acceptable salt thereof, wherein A, B, R¹, R², R³, m, and o are as defined herein.

In some embodiments, the compound of Formula I is a compound of Formula I-e:

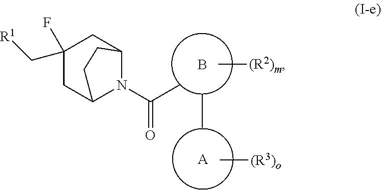

or a pharmaceutically acceptable salt thereof; wherein A, B, R¹, R², R³, m, and o are as defined herein.

In some embodiments, the compound of Formula I is a compound of Formula I-f:

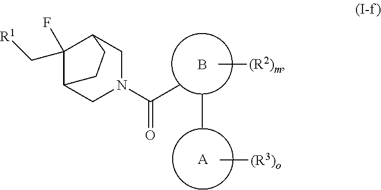

or a pharmaceutically acceptable salt thereof; wherein A, B, R¹, R², R³, m, and o are as defined herein.

### Group R1

In some embodiments, R¹ is substituted C₆-C₁₀ aryl. In some embodiments, R¹ is unsubstituted C₆-C₁₀ aryl. In some embodiments, R¹ is substituted C₆ aryl. In some embodiments, R¹ is unsubstituted C₆ aryl.

In some embodiments, R¹ is

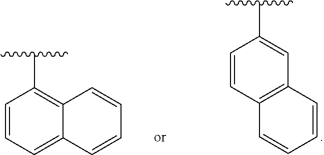 [SMILES: *c1cccc2ccccc12 ; *c1ccc2ccccc2c1]

In some embodiments, R¹ is

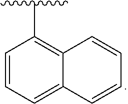 [SMILES: *c1cccc2ccccc12]

In some embodiments. R¹ is

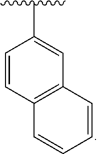 [SMILES: *c1ccc2ccccc2c1]

In some embodiments, R¹ is

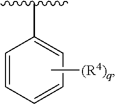

wherein each R⁴ is independently halo, —CN, —OH, —NO₂, —N(R⁵)₂, —S(O)₂R⁵, C₁-C₆ alkyl, C₁-C₆ haloalkyl, C₁-C₆ alkoxy, C₁-C₆ haloalkoxy, C₆-C₁₀ aryl, C₃-C₇ cycloalkyl, or 3-7 membered heterocyclyl; wherein each R⁵ is independently H or C₁-C₆ alkyl; and q is 0, 1, 2, or 3.

In some embodiments, R¹ is

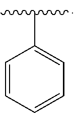 [SMILES: *c1ccccc1]

In some embodiments, R¹ is

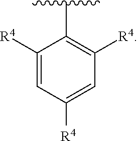

In some embodiments, R¹ is

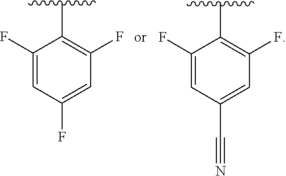

In some embodiments, R¹ is

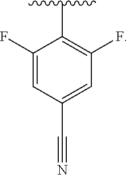 [SMILES: *c1c(F)cc(C#N)cc1F]

In certain embodiments, R¹ is

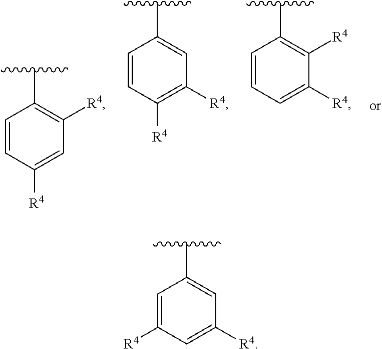

In some embodiments, R¹ is

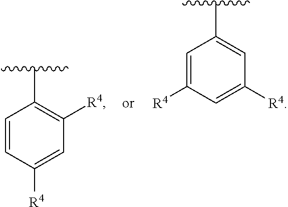

In some embodiments, R¹ is

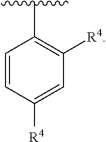

In some embodiments, R¹ is

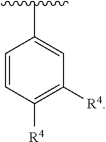

In some embodiments, R¹ is

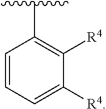

In some embodiments, R¹ is

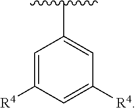

In some embodiments, R¹ is

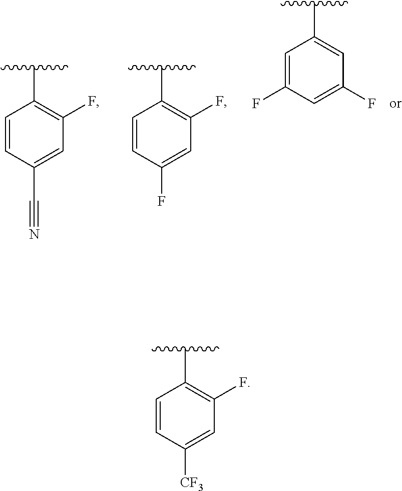

In some embodiments, R¹ is

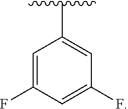 [SMILES: *c1cc(F)cc(F)c1]

In some embodiments, R¹ is

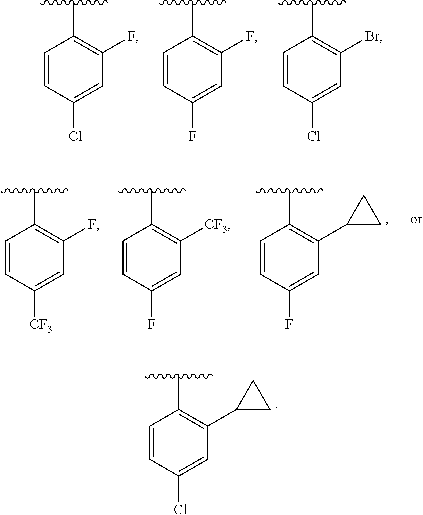

In some embodiments, R¹ is

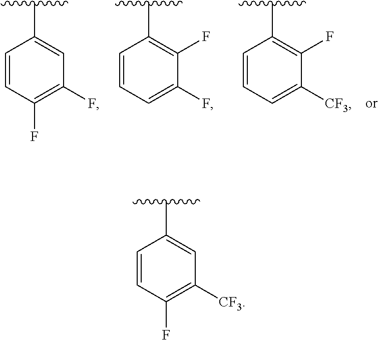

In some embodiment, R¹ is

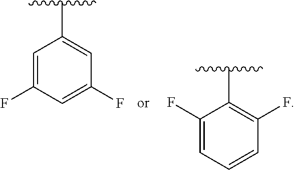 [SMILES: *c1cc(F)cc(F)c1 ; *c1c(F)cccc1F]

In some embodiment, R¹ is

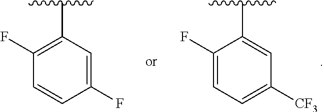 [SMILES: *c1cc(F)ccc1F ; *c1cc(C(F)(F)F)ccc1F]

In some embodiments, R¹ is

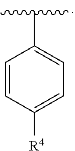

In some embodiments, R¹ is

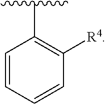

In some embodiments, R¹ is

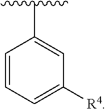

In some embodiments, R¹ is:

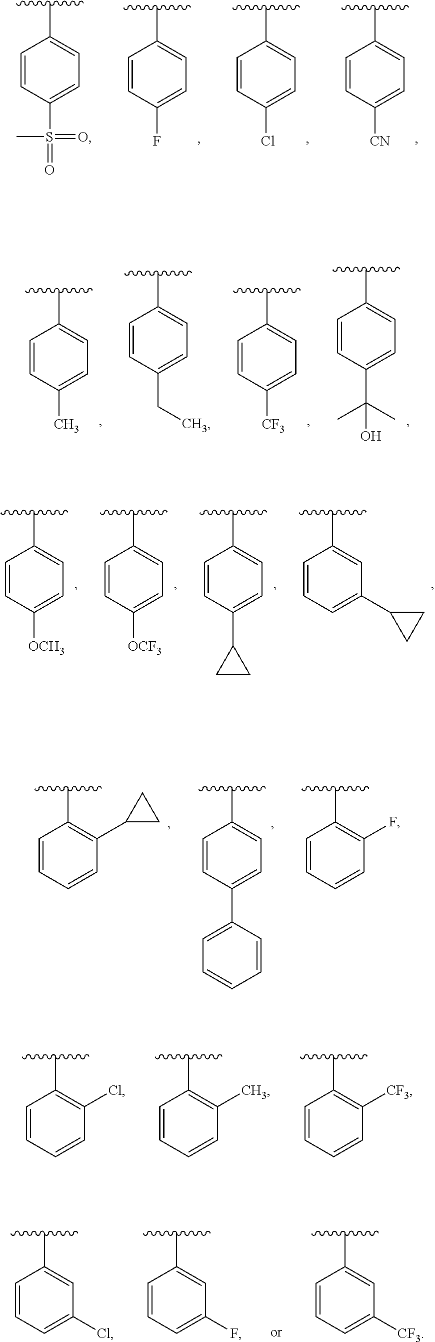

In some embodiments, R¹ is

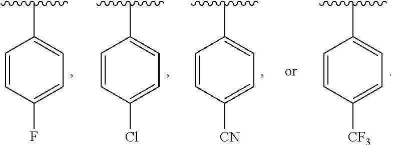

In some embodiments, R¹ is substituted 5-10 membered heteroaryl. In some embodiments, R¹ is unsubstituted 5-10 membered heteroaryl. In some embodiments, R¹ is a substituted 5-membered heteroaryl. In some embodiments, R¹ is a substituted 6-membered heteroaryl. In some embodiments, R¹ is an unsubstituted 5-membered heteroaryl. In some embodiments, R¹ is an unsubstituted 6-membered heteroaryl.

In some embodiments, R¹ is pyrrolyl, furanyl, thiophenyl, imidazolyl, pyrazolyl, oxazolyl, isoxazolyl, thiazolyl, isothiazolyl, triazolyl, oxadiazolyl, thiadiazolyl, pyridyl, 2-pyridyl, 3-pyridyl, 4-pyridyl, pyridinyl, pyridazinyl, pyrimidinyl, 2-pyrimidinyl, 4-pyrimidinyl, pyrazinyl, triazinyl, tetrazinyl, tetrazolyl, azocinyl, dithiazinyl, or oxazinyl. In some embodiments, R¹ is pyridyl, 2-pyridyl, 3-pyridyl, 4-pyridyl, pyridinyl, pyridazinyl, pyrimidinyl, 2-pyrimidinyl, 4-pyrimidinyl, pyridazinyl, or pyrazinyl. In some embodiments, R¹ is pyrrolyl, furanyl, thiophenyl, imidazolyl, pyrazolyl, oxazolyl, isoxazolyl, thiazolyl, isothiazolyl, triazolyl, oxadiazolyl, thiadiazolyl, pyridyl, pyridazinyl, pyrimidinyl, pyrazinyl, triazinyl, tetrazinyl, tetrazolyl, azocinyl, dithiazinyl, or oxazinyl. In some embodiments, R¹ is pyridyl, pyridinyl, pyridazinyl, pyrimidinyl, or pyrazinyl. In some embodiments, R¹ is 2-pyridyl, 3-pyridyl, 4-pyridyl, 2-pyrimidinyl, or 4-pyrimidinyl.

In some embodiments, R¹ is

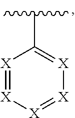

wherein each X is independently CH or N, wherein the H of CH may be substituted with one or more instances of R⁴; wherein each R⁴ is independently halo, —CN, —OH, —NO₂, —N(R⁵)₂, C₁-C₆ alkyl, C₁-C₆ haloalkyl, C₁-C₆ alkoxy, C₁-C₆ haloalkoxy, C₆-C₁₀ aryl, C₃-C₇ cycloalkyl, or 3-7 membered heterocyclyl; and each R⁵ is independently H or C₁-C₆ alkyl.

In some embodiments, R¹ is

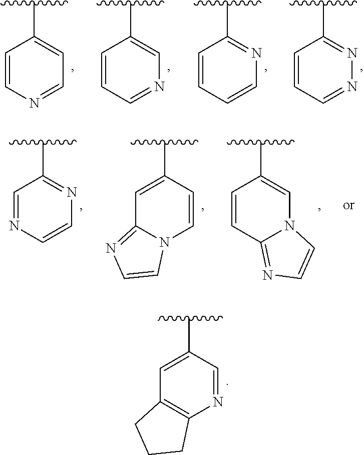

In some embodiments, R¹ is

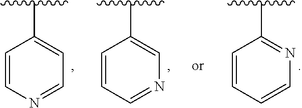

In some embodiments, R¹ is

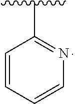 [SMILES: *c1ccccn1]

In some embodiments, R¹ is

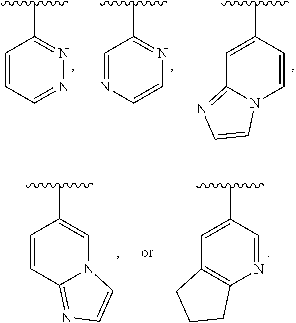

In some embodiments, R¹ is:

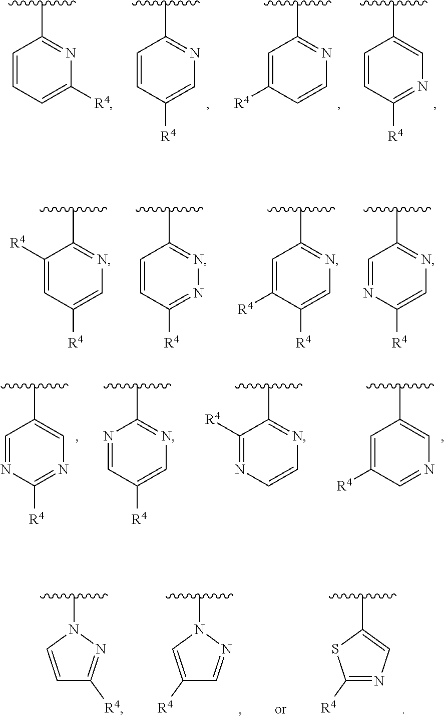

In some embodiments, R¹ is:

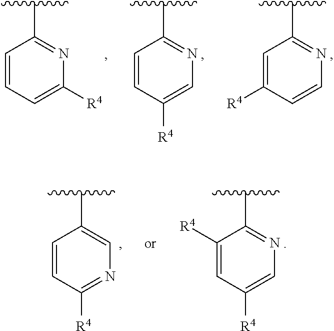

In some embodiments, R¹ is:

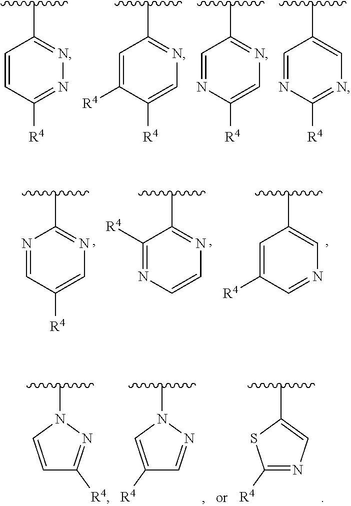

In some embodiments, R¹ is:

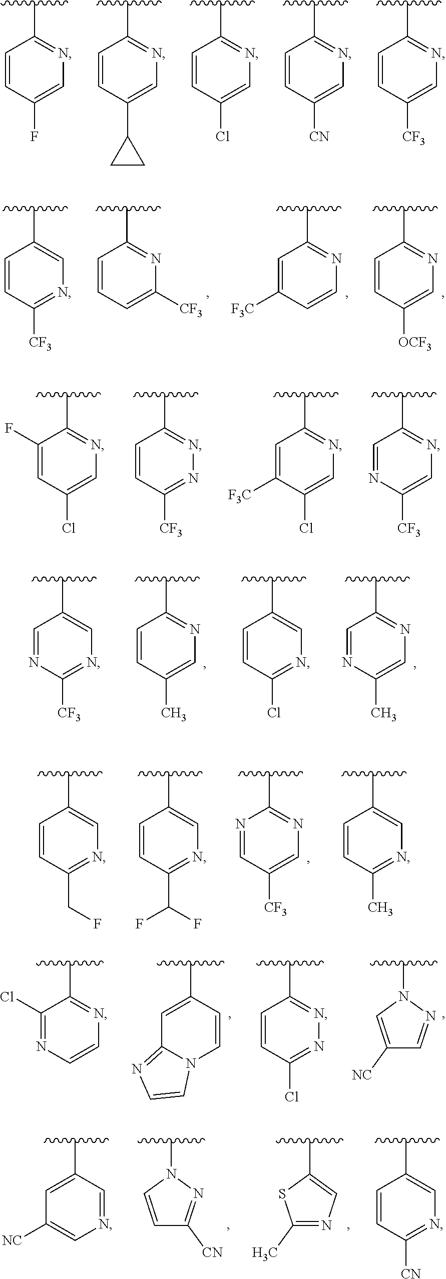

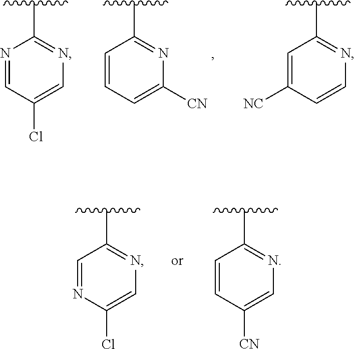

In some embodiments, R¹ is:

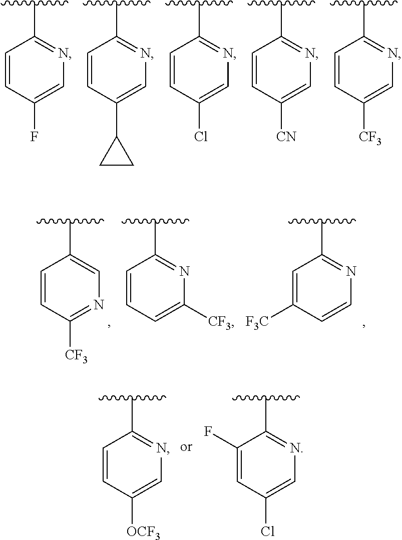

In some embodiments, R¹ is:

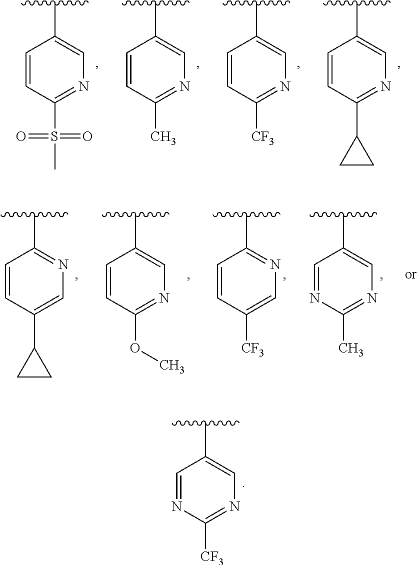

In some embodiments, R¹ is:

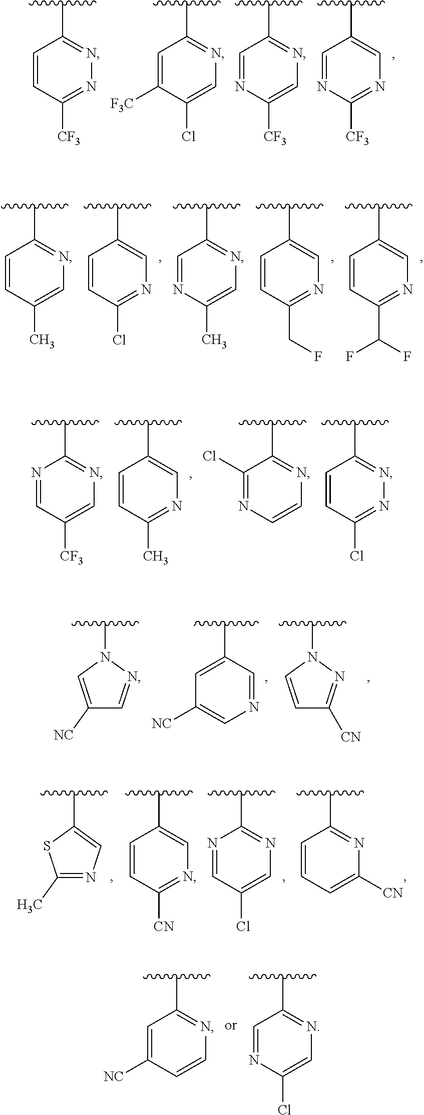

In some embodiments, R¹ is substituted C₃-C₇ cycloalkyl. In some embodiments, R¹ is unsubstituted C₃-C₇ cycloalkyl. In some embodiments, R¹ is cyclopropyl or cyclobutyl. In some embodiments, R¹ is

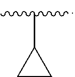 [SMILES: *C1CC1]

In some embodiments, R¹ is

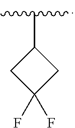 [SMILES: *C1CC(F)(F)C1]

In some embodiments, R¹ is substituted 3-7 membered heterocyclyl. In some embodiments, R¹ is unsubstituted 3-7 membered heterocyclyl. In some embodiments, R¹ is tetrahydrofuran, tetrahydropyran, pyrrolidine, piperidine, piperazine, dioxolane, dioxane, thiomorpholine, or dithiane. In some embodiments, R¹ is tetrahydrofuran or tetrahydropyran. In some embodiments, R¹ is

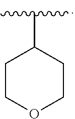 [SMILES: *C1CCOCC1]

### Group R⁴

In some embodiments, each R⁴ is independently halo, —CN, substituted C₁-C₆ alkyl, substituted C₁-C₆ alkoxy, or substituted C₃-C₇ cycloalkyl. In some embodiments, each R⁴ is independently halo, —CN, substituted C₁-C₆ alkyl, substituted C₁-C₆ alkoxy, or substituted C₃-C₇ cycloalkyl. In some embodiments, each R⁴ is independently halo, —CN, substituted C₁-C₆ alkyl, substituted C₁-C₆ alkoxy, or substituted C₃-C₇ cycloalkyl. In some embodiments, each R⁴ is independently halo, —CN, —CF₃, —OCF₃, or cyclopropyl. In some embodiments, each R⁴ is independently Cl, F, Br, or I. In some embodiments, each R⁴ is independently Cl, or F.

In some embodiments, each R⁴ is independently substituted C₁-C₆ alkyl, substituted C₁-C₆ alkoxy, or substituted C₃-C₇ cycloalkyl. In some embodiments, each R⁴ is independently unsubstituted C₁-C₆ alkyl, unsubstituted C₁-C₆ alkoxy, or unsubstituted C₃-C₇ cycloalkyl. In some embodiments, each R⁴ is independently halo, —NH₂, —NH(C₁-C₆ alkyl), —N(C₁-C₆ alkyl)₂, C₁-C₆ alkyl, C₁-C₆ haloalkyl, C₁-C₆ alkoxy, C₁-C₆ haloalkoxy, or C₆-C₁₀ aryl. In some embodiments, each R⁴ is independently halo, C₁-C₆ alkyl, C₁-C₆ haloalkyl, C₁-C₆ alkoxy, C₁-C₆ haloalkoxy, or C₆-C₁₀ aryl. In some embodiments, each R⁴ is independently halo, —CN, —CH₃, —CH₂CH₃, —CF₃, —OCH₃, —OCF₃, —C(CH₃)₂₀H, or —C₆H₅.

In some embodiments, each R⁴ is independently halo, substituted or unsubstituted C₁-C₆ alkyl, or —CN. In some embodiments, each R⁴ is independently halo, —CN, unsubstituted C₁-C₆ alkyl, or C₁-C₆ haloalkyl. In some embodiments, each R⁴ is independently halo, —CN, —CH₃, —CF₃, —CH₂F, or —CHF₂. In some embodiments, each R⁴ is independently F, Cl, —CN, —CH₃, —CF₃, —CH₂F, or —CHF₂.

In some embodiments, each R⁴ is independently halo, —CN, substituted or unsubstituted C₁-C₆ alkyl, substituted or unsubstituted C₁-C₆ alkoxy, or substituted or unsubstituted C₃-C₇ cycloalkyl. In some embodiments, each R⁴ is independently halo, —CN, unsubstituted C₁-C₆ alkyl, C₁-C₆ haloalkyl, unsubstituted C₁-C₆ alkoxy, C₁-C₆ haloalkoxy, or unsubstituted C₃-C₇ cycloalkyl. In some embodiments, each R⁴ is independently halo, —CN, —CH₃, —CF₃, —CH₂F, —CHF₂, —OCH₃, —OCF₃, or cyclopropyl. In some embodiments, each R⁴ is independently —F, —Cl, —CN, —CH₃, —CF₃, —CH₂F, —CHF₂, —OCF₃, or cyclopropyl.

### Group B

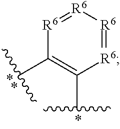

In some embodiments, B is; herein each R⁶ is independently N or CR^{6a}, wherein R^{6a} is H or R²; and ** is the point of attachment to the carbonyl, and * is the point of attachment to A.

In some embodiments, R² is halo, —CN, —OH, —NO₂, —CH₃, —CH₂CH₃, —CHF₂, —CF₃, —OCF₃, —OCH₃, —OCH₂CH₃, or —OCH₂CF₃. In some embodiments, up to two R⁶ may be N and the other occurrences of R⁶ are CH.

In some embodiments, B is:

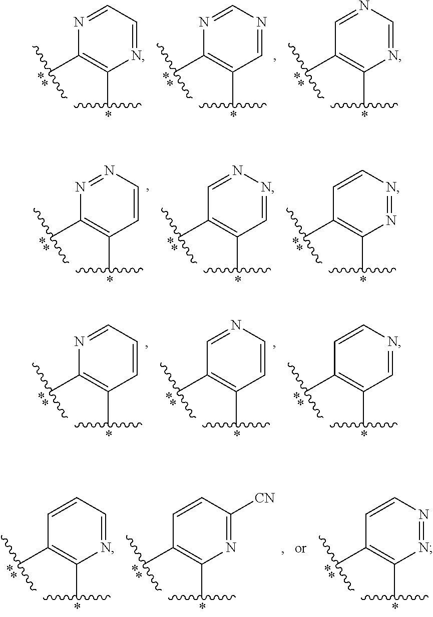

wherein ** is the point of attachment to a carbonyl, and * is the point of attachment to A.

In some embodiments, B is:

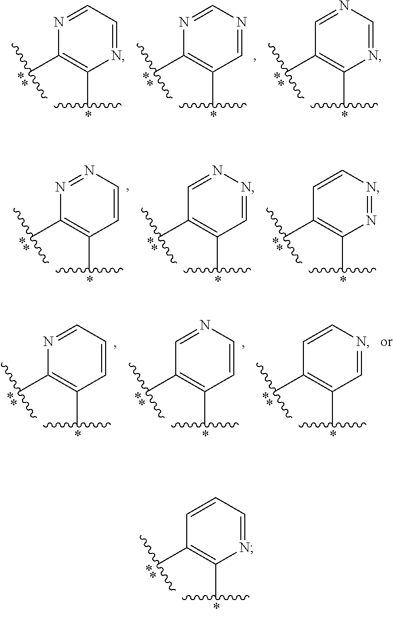

wherein ** is the point of attachment to a carbonyl, and * is the point of attachment to A.

In some embodiments, B is,

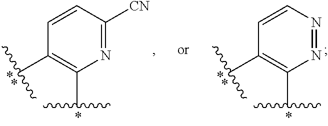 [SMILES: *c1ccc(C#N)nc1* ; *c1ccnnc1*]

wherein ** is the point of attachment to the carbonyl, and * is the point of attachment to A.

In some embodiments, B is

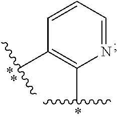 [SMILES: *c1cccnc1*]

wherein ** is the point of attachment to the carbonyl, and * is the point of attachment to A. In some embodiments, B is

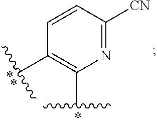 [SMILES: *c1ccc(C#N)nc1*]

wherein ** is the point of attachment to the carbonyl, and * is the point of attachment to A.

In some embodiments, B is

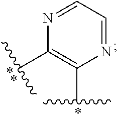 [SMILES: *c1nccnc1*]

wherein ** is the point of attachment to the carbonyl, and * is the point of attachment to A.

In some embodiments, B is

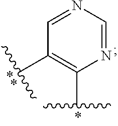 [SMILES: *c1cncnc1*]

wherein ** is the point of attachment to the carbonyl, and * is the point of attachment to A.

In some embodiments, B is

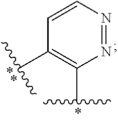 [SMILES: *c1ccnnc1*]

wherein ** is the point of attachment to the carbonyl, and * is the point of attachment to A.

In some embodiments, B is

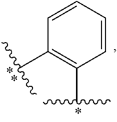 [SMILES: *c1ccccc1*]

wherein ** is the point of attachment to a carbonyl, and * is the point of attachment to A. In some embodiments, B is

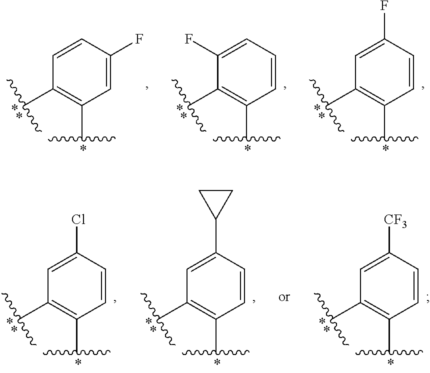 [SMILES: *c1ccc(F)cc1* ; *c1cccc(F)c1* ; *c1ccc(F)cc1* ; *c1ccc(Cl)cc1* ; *c1ccc(C2CC2)cc1* ; *c1ccc(C(F)(F)F)cc1*]

wherein ** is the point of attachment to a carbonyl, and * is the point of attachment to A.

### Group R²

In some embodiments, R² is halo, —CN, —OH, —NO₂, —CH₃, —CH₂CH₃, cyclopropyl, —CHF₂, —CF₃, —OCF₃, —OCH₃, —OCH₂CH₃, or —OCH₂CF₃. In some embodiments, R² is —CN.

### Group A

In some embodiments, A is pyridinyl, pyrrolyl, imidazolyl, pyrazolyl, pyridazinyl, pyrimidinyl, pyrazinyl, triazolyl, triazinyl, tetrazinyl, tetrazolyl, oxazolyl, isoxazolyl, or thiozolyl. In embodiments, A is pyridinyl oxazolyl, imidazolyl, triazolyl, pyridinyl, pyridazinyl, pyrimidinyl, or triazinyl. In some embodiments, A is

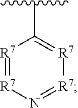

wherein each R⁷ is independently N or CH, wherein up to two R⁷ may be N and the other occurrences of R⁷ are CH. In some embodiments, the hydrogen of CH may be substituted with R³.

In some embodiments, A is

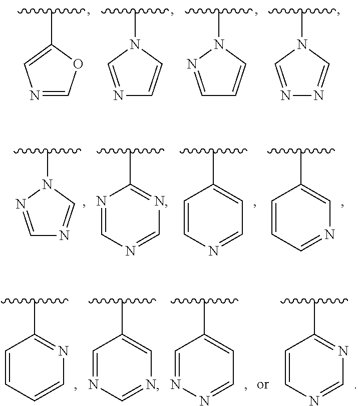

In some embodiments, A is

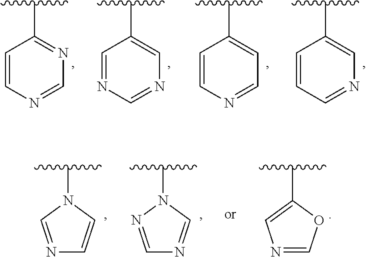

In some embodiments, A is

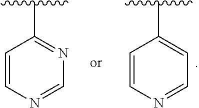 [SMILES: *c1ccncn1 ; *c1ccncc1]

n

In some embodiments, n is 4. In some embodiments, n is 3. In some embodiments, n is 2. In some embodiments, n is 1. In some embodiments, n is 0.

In some embodiments, n is 1, and R^{a} is C₁-C₆ alkyl and R^{b} is H. In some embodiments, n is 1, and R^{a} is ethyl and R^{b} is H. In some embodiments, n is 1, and R^{a} is methyl and R^{b} is H. In some embodiments, n is 1, R^{a} is —OH, and R^{b} is H. In some embodiments, n is 1, and R^{a} and R^{b} are taken together to form an oxo. In some embodiments, n is 1, and R^{a} and R^{b} are both H. In some embodiments, n is 1, and R^{a} and R^{b} form together with the carbon to which they are attached, a cyclopropyl.

m

In some embodiments, m is 3. In some embodiments, m is 2. In some embodiments, m is 1. In some embodiments, m is 0.

o

In some embodiments, o is 3. In some embodiments, o is 2. In some embodiments, o is 1. In some embodiments, o is 0.

p

In some embodiments, p is 2. In some embodiments, p is 1. In some embodiments, p is 0.

In some embodiments, p is 1, and R^{c}, R^{d}, R^{e}, and R^{f} are H. In some embodiments, p is 1, R^{c} is methyl, and R^{d}, R^{e}, and R^{f} are H. In some embodiments, p is 1, R^{c} and R^{e} are H, and R^{d} and R^{f} form together with the carbon to which they are attached, an C₁-C₃ alkylene bridge. In some embodiments, p is 1, R^{d} and R^{f} are H, and R^{c} and R^{e} form together with the carbon to which they are attached, an C₁-C₃ alkylene bridge. In some embodiments, p is 0, and R^{c}, R^{d}, and R^{f} are H.

q

In some embodiments, q is 3. In some embodiments, q is 2. In some embodiments, q is 1. In some embodiments, q is 0.

In some embodiments, the compound is any one of the compounds in Table 1.

TABLE 1
Exemplary compounds 1-98.

| Cmpd | |
|---|---|
| # | Structure |
| 1 | 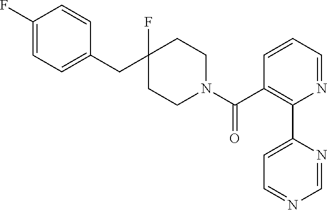 [SMILES: O=C(c1cccnc1-c1ccncn1)N1CCC(F)(Cc2ccc(F)cc2)CC1] |
| 2 | 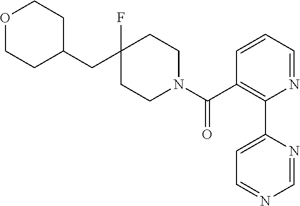 [SMILES: O=C(c1cccnc1-c1ccncn1)N1CCC(F)(CC2CCOCC2)CC1] |
| 3 | 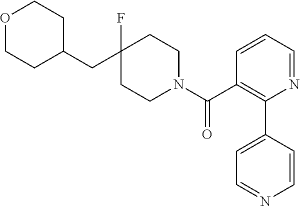 [SMILES: O=C(c1cccnc1-c1ccncc1)N1CCC(F)(CC2CCOCC2)CC1] |
| 4 | 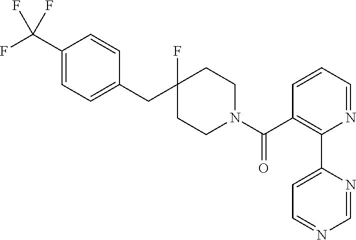 [SMILES: O=C(c1cccnc1-c1ccncn1)N1CCC(F)(Cc2ccc(C(F)(F)F)cc2)CC1] |
| 5 | 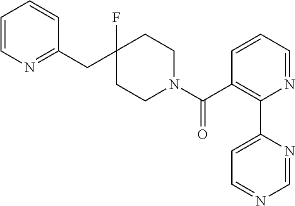 [SMILES: O=C(c1cccnc1-c1ccncn1)N1CCC(F)(Cc2ccccn2)CC1] |
| 6 | 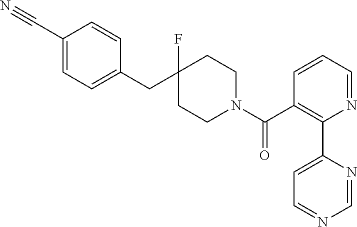 [SMILES: N#Cc1ccc(CC2(F)CCN(C(=O)c3cccnc3-c3ccncn3)CC2)cc1] |
| 7 | 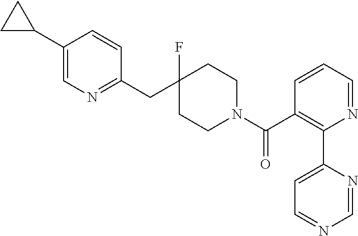 [SMILES: O=C(c1cccnc1-c1ccncn1)N1CCC(F)(Cc2ccc(C3CC3)cn2)CC1] |
| 8 | 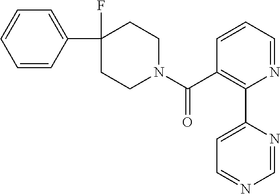 [SMILES: O=C(c1cccnc1-c1ccncn1)N1CCC(F)(c2ccccc2)CC1] |
| 9 | 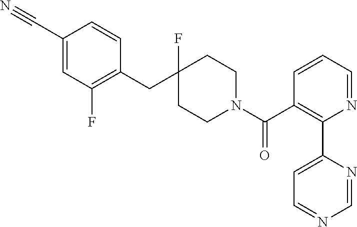 [SMILES: N#Cc1ccc(CC2(F)CCN(C(=O)c3cccnc3-c3ccncn3)CC2)c(F)c1] |
| 10 | 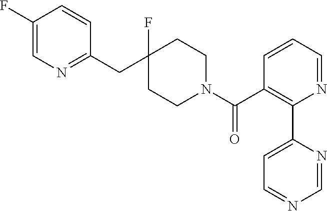 [SMILES: O=C(c1cccnc1-c1ccncn1)N1CCC(F)(Cc2ccc(F)cn2)CC1] |
| 11 | 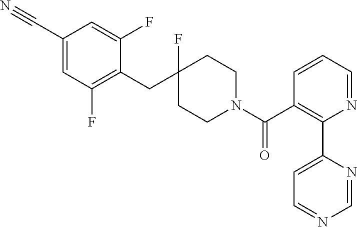 [SMILES: N#Cc1cc(F)c(CC2(F)CCN(C(=O)c3cccnc3-c3ccncn3)CC2)c(F)c1] |
| 12 | 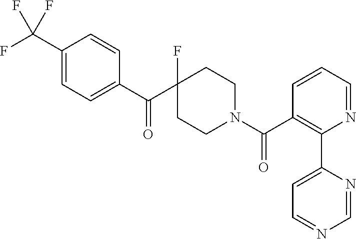 [SMILES: O=C(c1cccnc1-c1ccncn1)N1CCC(F)(C(=O)c2ccc(C(F)(F)F)cc2)CC1] |
| 13 | 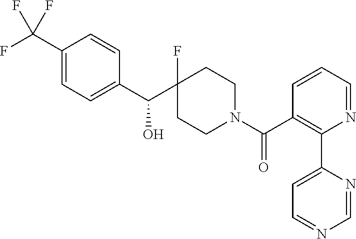 [SMILES: O=C(c1cccnc1-c1ccncn1)N1CCC(F)([C@H](O)c2ccc(C(F)(F)F)cc2)CC1] |
| 14 | 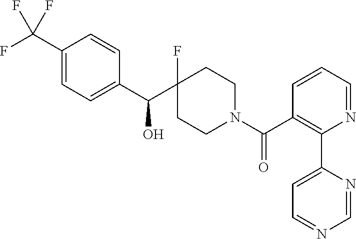 [SMILES: O=C(c1cccnc1-c1ccncn1)N1CCC(F)([C@@H](O)c2ccc(C(F)(F)F)cc2)CC1] |
| 15 | 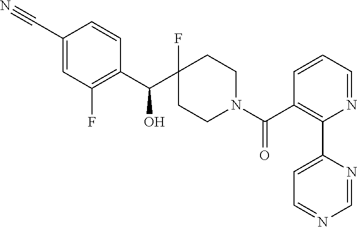 [SMILES: N#Cc1ccc([C@H](O)C2(F)CCN(C(=O)c3cccnc3-c3ccncn3)CC2)c(F)c1] |
| 16 | 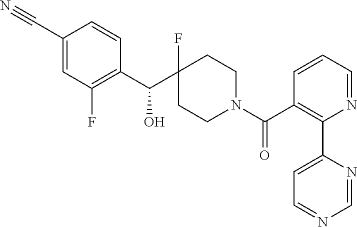 [SMILES: N#Cc1ccc([C@@H](O)C2(F)CCN(C(=O)c3cccnc3-c3ccncn3)CC2)c(F)c1] |
| 17 | 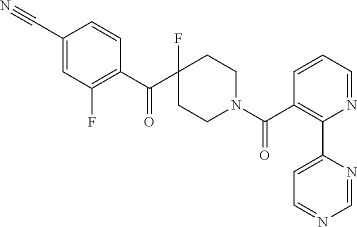 [SMILES: N#Cc1ccc(C(=O)C2(F)CCN(C(=O)c3cccnc3-c3ccncn3)CC2)c(F)c1] |
| 18 | 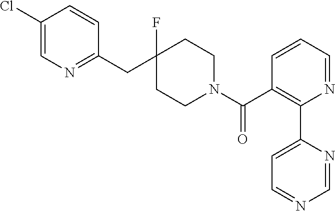 [SMILES: O=C(c1cccnc1-c1ccncn1)N1CCC(F)(Cc2ccc(Cl)cn2)CC1] |
| 19 | 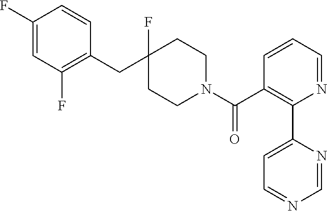 [SMILES: O=C(c1cccnc1-c1ccncn1)N1CCC(F)(Cc2ccc(F)cc2F)CC1] |
| 20 | 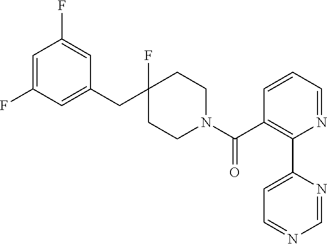 [SMILES: O=C(c1cccnc1-c1ccncn1)N1CCC(F)(Cc2cc(F)cc(F)c2)CC1] |
| 21 | 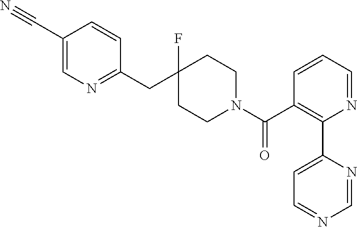 [SMILES: N#Cc1ccc(CC2(F)CCN(C(=O)c3cccnc3-c3ccncn3)CC2)nc1] |
| 22 | 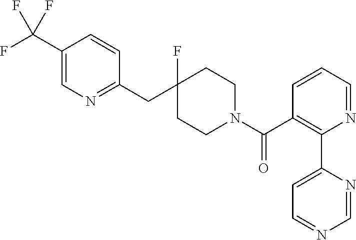 [SMILES: O=C(c1cccnc1-c1ccncn1)N1CCC(F)(Cc2ccc(C(F)(F)F)cn2)CC1] |
| 23 | 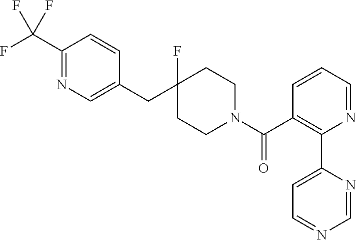 [SMILES: O=C(c1cccnc1-c1ccncn1)N1CCC(F)(Cc2ccc(C(F)(F)F)nc2)CC1] |
| 24 | 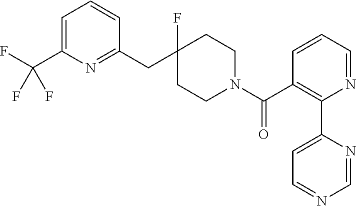 [SMILES: O=C(c1cccnc1-c1ccncn1)N1CCC(F)(Cc2cccc(C(F)(F)F)n2)CC1] |
| 25 | 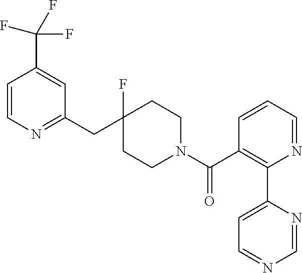 [SMILES: O=C(c1cccnc1-c1ccncn1)N1CCC(F)(Cc2cc(C(F)(F)F)ccn2)CC1] |
| 26 | 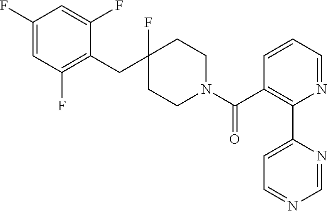 [SMILES: O=C(c1cccnc1-c1ccncn1)N1CCC(F)(Cc2c(F)cc(F)cc2F)CC1] |
| 27 | 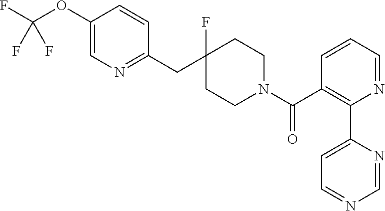 [SMILES: O=C(c1cccnc1-c1ccncn1)N1CCC(F)(Cc2ccc(OC(F)(F)F)cn2)CC1] |
| 28 | 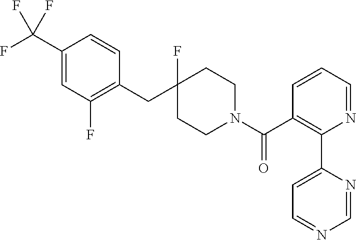 [SMILES: O=C(c1cccnc1-c1ccncn1)N1CCC(F)(Cc2ccc(C(F)(F)F)cc2F)CC1] |
| 29 | 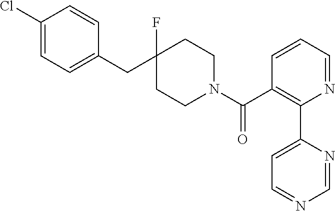 [SMILES: O=C(c1cccnc1-c1ccncn1)N1CCC(F)(Cc2ccc(Cl)cc2)CC1] |
| 30 | 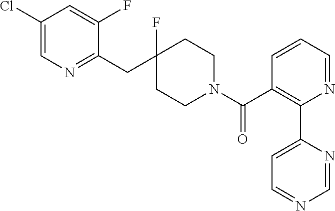 [SMILES: O=C(c1cccnc1-c1ccncn1)N1CCC(F)(Cc2ncc(Cl)cc2F)CC1] |
| 31 | 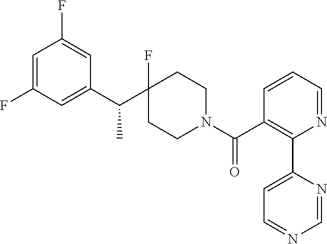 [SMILES: C[C@H](c1cc(F)cc(F)c1)C1(F)CCN(C(=O)c2cccnc2-c2ccncn2)CC1] |
| 32 | 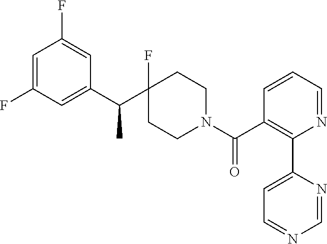 [SMILES: C[C@@H](c1cc(F)cc(F)c1)C1(F)CCN(C(=O)c2cccnc2-c2ccncn2)CC1] |
| 33 | 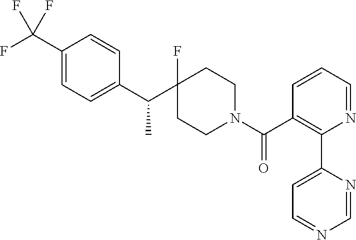 [SMILES: C[C@H](c1ccc(C(F)(F)F)cc1)C1(F)CCN(C(=O)c2cccnc2-c2ccncn2)CC1] |
| 34 | 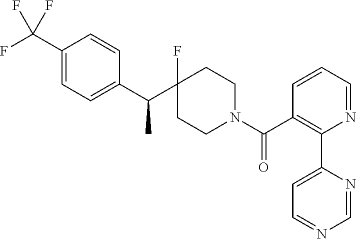 [SMILES: C[C@@H](c1ccc(C(F)(F)F)cc1)C1(F)CCN(C(=O)c2cccnc2-c2ccncn2)CC1] |
| 35 | 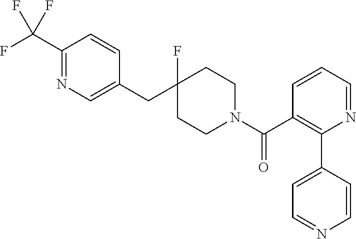 [SMILES: O=C(c1cccnc1-c1ccncc1)N1CCC(F)(Cc2ccc(C(F)(F)F)nc2)CC1] |
| 36 | 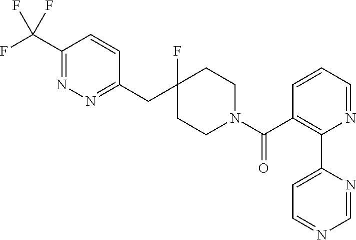 [SMILES: O=C(c1cccnc1-c1ccncn1)N1CCC(F)(Cc2ccc(C(F)(F)F)nn2)CC1] |
| 37 | 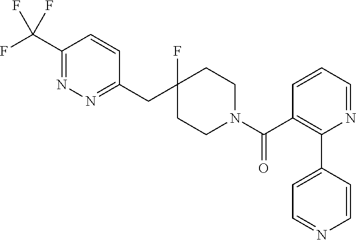 [SMILES: O=C(c1cccnc1-c1ccncc1)N1CCC(F)(Cc2ccc(C(F)(F)F)nn2)CC1] |
| 38 | 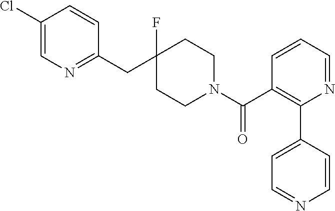 [SMILES: O=C(c1cccnc1-c1ccncc1)N1CCC(F)(Cc2ccc(Cl)cn2)CC1] |
| 39 | 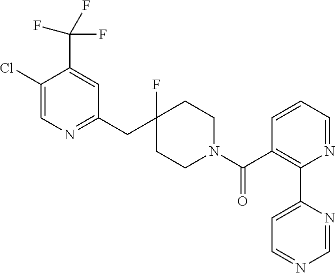 [SMILES: O=C(c1cccnc1-c1ccncn1)N1CCC(F)(Cc2cc(C(F)(F)F)c(Cl)cn2)CC1] |
| 40 | 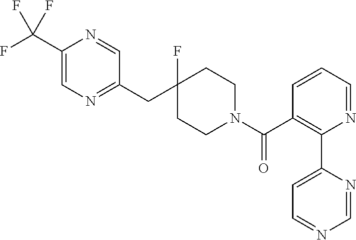 [SMILES: O=C(c1cccnc1-c1ccncn1)N1CCC(F)(Cc2cnc(C(F)(F)F)cn2)CC1] |
| 41 | 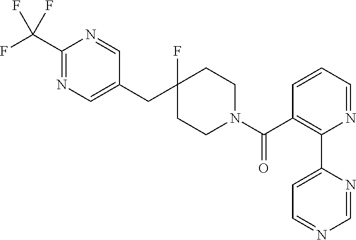 [SMILES: O=C(c1cccnc1-c1ccncn1)N1CCC(F)(Cc2cnc(C(F)(F)F)nc2)CC1] |
| 42 | 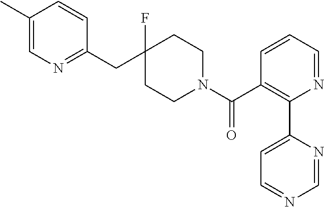 [SMILES: Cc1ccc(CC2(F)CCN(C(=O)c3cccnc3-c3ccncn3)CC2)nc1] |
| 43 | 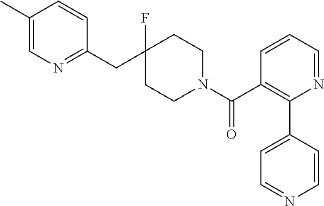 [SMILES: Cc1ccc(CC2(F)CCN(C(=O)c3cccnc3-c3ccncc3)CC2)nc1] |
| 44 | 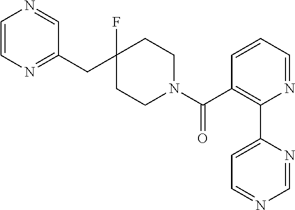 [SMILES: O=C(c1cccnc1-c1ccncn1)N1CCC(F)(Cc2cnccn2)CC1] |
| 45 | 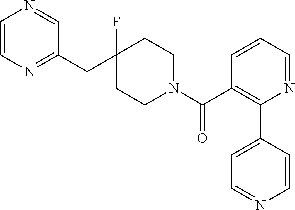 [SMILES: O=C(c1cccnc1-c1ccncc1)N1CCC(F)(Cc2cnccn2)CC1] |
| 46 | 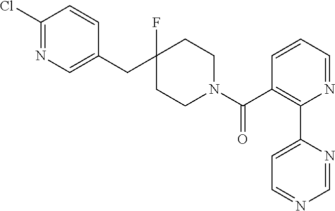 [SMILES: O=C(c1cccnc1-c1ccncn1)N1CCC(F)(Cc2ccc(Cl)nc2)CC1] |
| 47 | 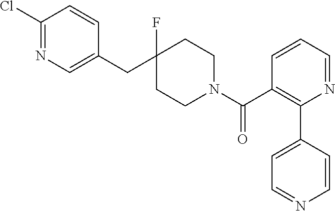 [SMILES: O=C(c1cccnc1-c1ccncc1)N1CCC(F)(Cc2ccc(Cl)nc2)CC1] |
| 48 | 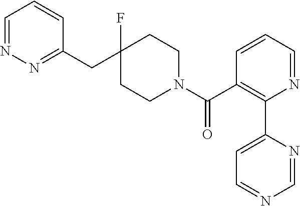 [SMILES: O=C(c1cccnc1-c1ccncn1)N1CCC(F)(Cc2cccnn2)CC1] |
| 49 | 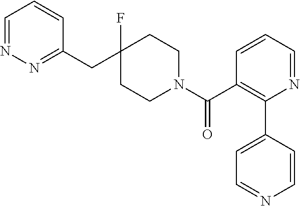 [SMILES: O=C(c1cccnc1-c1ccncc1)N1CCC(F)(Cc2cccnn2)CC1] |
| 50 | 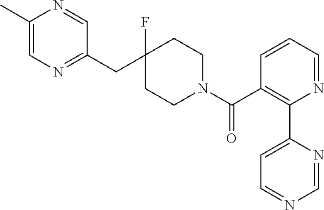 [SMILES: Cc1cnc(CC2(F)CCN(C(=O)c3cccnc3-c3ccncn3)CC2)cn1] |
| 51 | 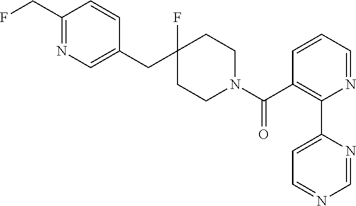 [SMILES: O=C(c1cccnc1-c1ccncn1)N1CCC(F)(Cc2ccc(CF)nc2)CC1] |
| 52 | 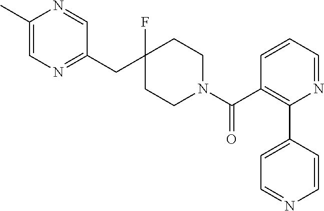 [SMILES: Cc1cnc(CC2(F)CCN(C(=O)c3cccnc3-c3ccncc3)CC2)cn1] |
| 53 | 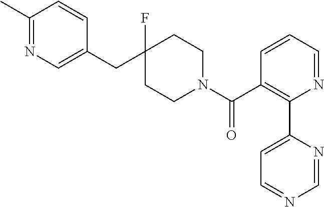 [SMILES: Cc1ccc(CC2(F)CCN(C(=O)c3cccnc3-c3ccncn3)CC2)cn1] |
| 54 | 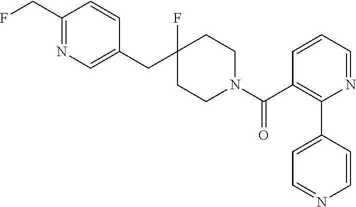 [SMILES: O=C(c1cccnc1-c1ccncc1)N1CCC(F)(Cc2ccc(CF)nc2)CC1] |
| 55 | 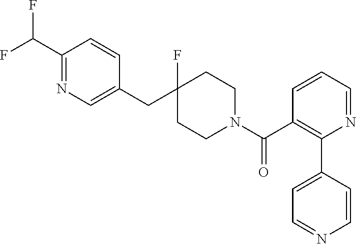 [SMILES: O=C(c1cccnc1-c1ccncc1)N1CCC(F)(Cc2ccc(C(F)F)nc2)CC1] |
| 56 | 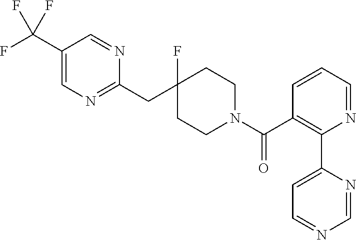 [SMILES: O=C(c1cccnc1-c1ccncn1)N1CCC(F)(Cc2ncc(C(F)(F)F)cn2)CC1] |
| 57 | 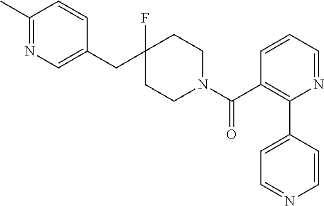 [SMILES: Cc1ccc(CC2(F)CCN(C(=O)c3cccnc3-c3ccncc3)CC2)cn1] |
| 58 | 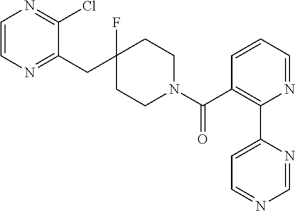 [SMILES: O=C(c1cccnc1-c1ccncn1)N1CCC(F)(Cc2nccnc2Cl)CC1] |
| 59 | 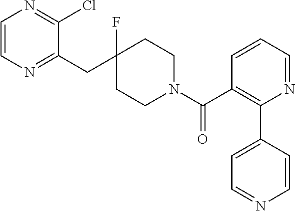 [SMILES: O=C(c1cccnc1-c1ccncc1)N1CCC(F)(Cc2nccnc2Cl)CC1] |
| 60 | 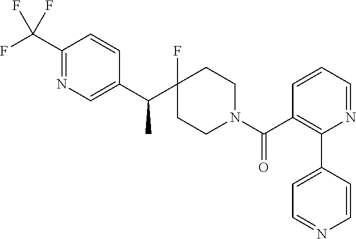 [SMILES: C[C@@H](c1ccc(C(F)(F)F)nc1)C1(F)CCN(C(=O)c2cccnc2-c2ccncc2)CC1] |
| 61 | 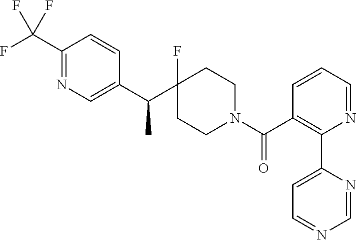 [SMILES: C[C@@H](c1ccc(C(F)(F)F)nc1)C1(F)CCN(C(=O)c2cccnc2-c2ccncn2)CC1] |
| 62 | 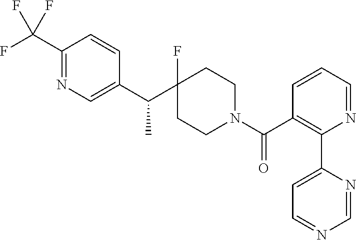 [SMILES: C[C@H](c1ccc(C(F)(F)F)nc1)C1(F)CCN(C(=O)c2cccnc2-c2ccncn2)CC1] |
| 63 | 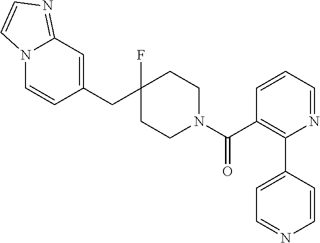 [SMILES: O=C(c1cccnc1-c1ccncc1)N1CCC(F)(Cc2ccn3ccnc3c2)CC1] |
| 64 | 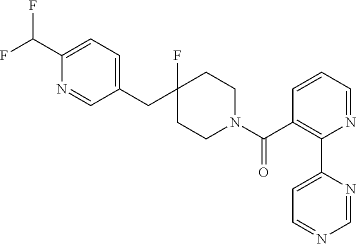 [SMILES: O=C(c1cccnc1-c1ccncn1)N1CCC(F)(Cc2ccc(C(F)F)nc2)CC1] |
| 65 | 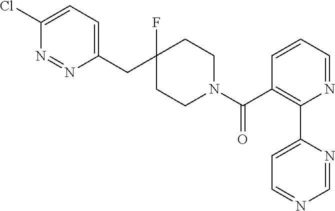 [SMILES: O=C(c1cccnc1-c1ccncn1)N1CCC(F)(Cc2ccc(Cl)nn2)CC1] |
| 66 | 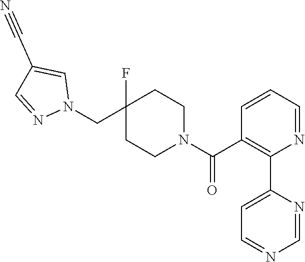 [SMILES: N#Cc1cnn(CC2(F)CCN(C(=O)c3cccnc3-c3ccncn3)CC2)c1] |
| 67 | 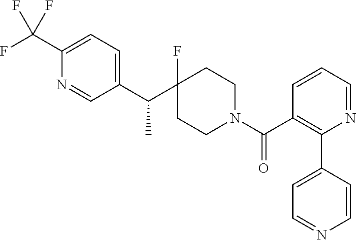 [SMILES: C[C@H](c1ccc(C(F)(F)F)nc1)C1(F)CCN(C(=O)c2cccnc2-c2ccncc2)CC1] |
| 68 | 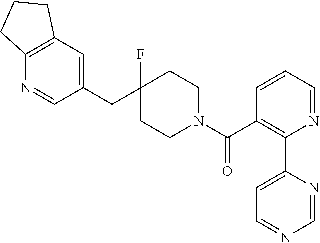 [SMILES: O=C(c1cccnc1-c1ccncn1)N1CCC(F)(Cc2cnc3c(c2)CCC3)CC1] |
| 69 | 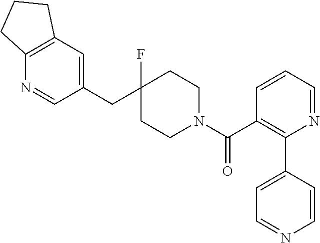 [SMILES: O=C(c1cccnc1-c1ccncc1)N1CCC(F)(Cc2cnc3c(c2)CCC3)CC1] |
| 70 | 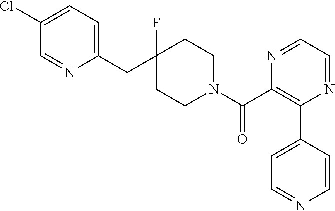 [SMILES: O=C(c1nccnc1-c1ccncc1)N1CCC(F)(Cc2ccc(Cl)cn2)CC1] |
| 71 | 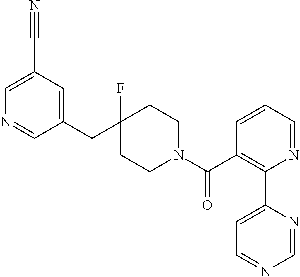 [SMILES: N#Cc1cncc(CC2(F)CCN(C(=O)c3cccnc3-c3ccncn3)CC2)c1] |
| 72 | 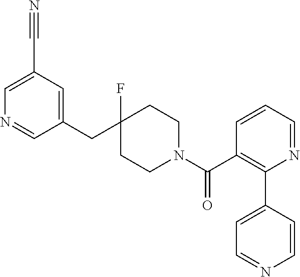 [SMILES: N#Cc1cncc(CC2(F)CCN(C(=O)c3cccnc3-c3ccncc3)CC2)c1] |
| 73 | 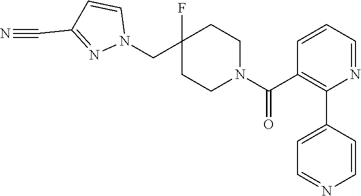 [SMILES: N#Cc1ccn(CC2(F)CCN(C(=O)c3cccnc3-c3ccncc3)CC2)n1] |
| 74 | 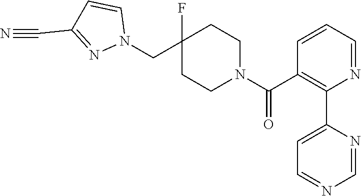 [SMILES: N#Cc1ccn(CC2(F)CCN(C(=O)c3cccnc3-c3ccncn3)CC2)n1] |
| 75 | 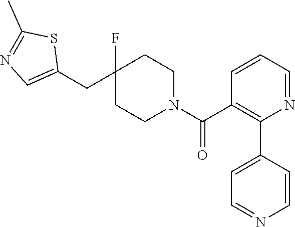 [SMILES: Cc1ncc(CC2(F)CCN(C(=O)c3cccnc3-c3ccncc3)CC2)s1] |
| 76 | 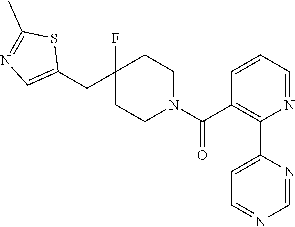 [SMILES: Cc1ncc(CC2(F)CCN(C(=O)c3cccnc3-c3ccncn3)CC2)s1] |
| 77 | 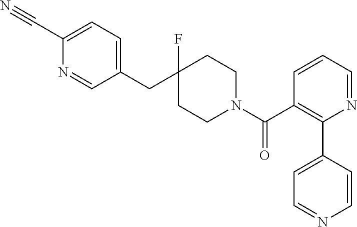 [SMILES: N#Cc1ccc(CC2(F)CCN(C(=O)c3cccnc3-c3ccncc3)CC2)cn1] |
| 78 | 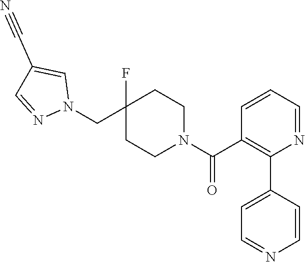 [SMILES: N#Cc1cnn(CC2(F)CCN(C(=O)c3cccnc3-c3ccncc3)CC2)c1] |
| 79 | 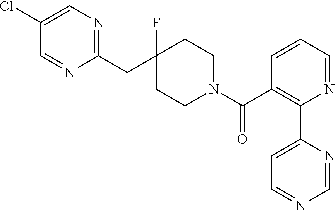 [SMILES: O=C(c1cccnc1-c1ccncn1)N1CCC(F)(Cc2ncc(Cl)cn2)CC1] |
| 80 | 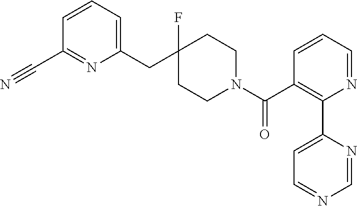 [SMILES: N#Cc1cccc(CC2(F)CCN(C(=O)c3cccnc3-c3ccncn3)CC2)n1] |
| 81 | 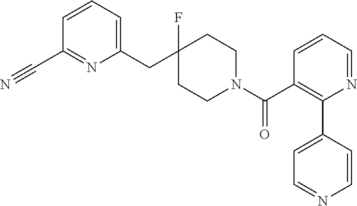 [SMILES: N#Cc1cccc(CC2(F)CCN(C(=O)c3cccnc3-c3ccncc3)CC2)n1] |
| 82 | 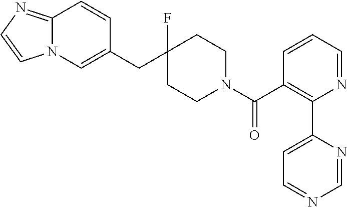 [SMILES: O=C(c1cccnc1-c1ccncn1)N1CCC(F)(Cc2ccc3nccn3c2)CC1] |
| 83 | 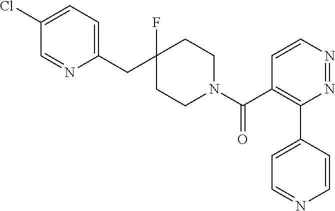 [SMILES: O=C(c1ccnnc1-c1ccncc1)N1CCC(F)(Cc2ccc(Cl)cn2)CC1] |
| 84 | 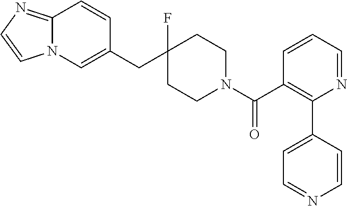 [SMILES: O=C(c1cccnc1-c1ccncc1)N1CCC(F)(Cc2ccc3nccn3c2)CC1] |
| 85 | 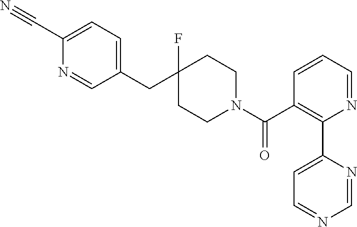 [SMILES: N#Cc1ccc(CC2(F)CCN(C(=O)c3cccnc3-c3ccncn3)CC2)cn1] |
| 86 | 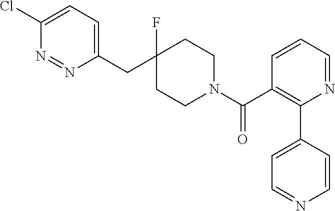 [SMILES: O=C(c1cccnc1-c1ccncc1)N1CCC(F)(Cc2ccc(Cl)nn2)CC1] |
| 87 | 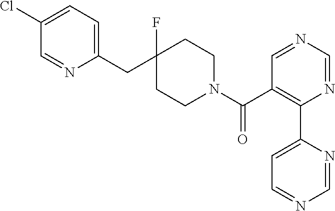 [SMILES: O=C(c1cncnc1-c1ccncn1)N1CCC(F)(Cc2ccc(Cl)cn2)CC1] |
| 88 | 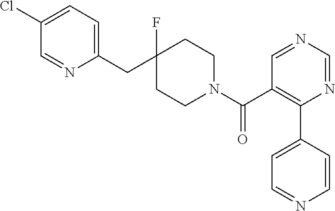 [SMILES: O=C(c1cncnc1-c1ccncc1)N1CCC(F)(Cc2ccc(Cl)cn2)CC1] |
| 89 | 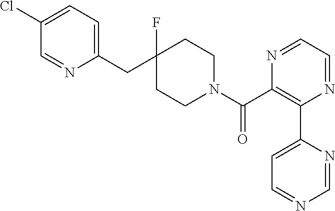 [SMILES: O=C(c1nccnc1-c1ccncn1)N1CCC(F)(Cc2ccc(Cl)cn2)CC1] |
| 90 | 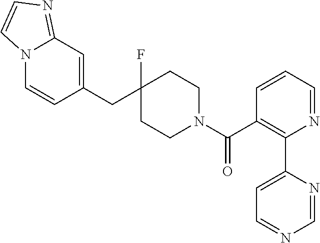 [SMILES: O=C(c1cccnc1-c1ccncn1)N1CCC(F)(Cc2ccn3ccnc3c2)CC1] |
| 91 | 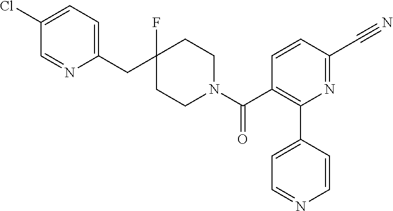 [SMILES: N#Cc1ccc(C(=O)N2CCC(F)(Cc3ccc(Cl)cn3)CC2)c(-c2ccncc2)n1] |
| 92 | 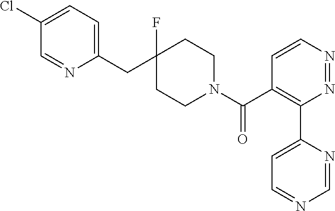 [SMILES: O=C(c1ccnnc1-c1ccncn1)N1CCC(F)(Cc2ccc(Cl)cn2)CC1] |
| 93 | 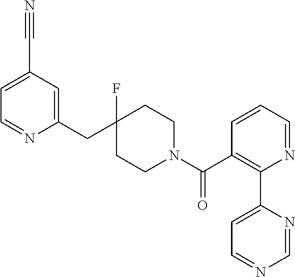 [SMILES: N#Cc1ccnc(CC2(F)CCN(C(=O)c3cccnc3-c3ccncn3)CC2)c1] |
| 94 | 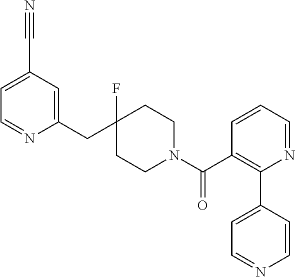 [SMILES: N#Cc1ccnc(CC2(F)CCN(C(=O)c3cccnc3-c3ccncc3)CC2)c1] |
| 95 | 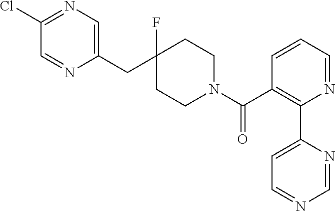 [SMILES: O=C(c1cccnc1-c1ccncn1)N1CCC(F)(Cc2cnc(Cl)cn2)CC1] |
| 96 | 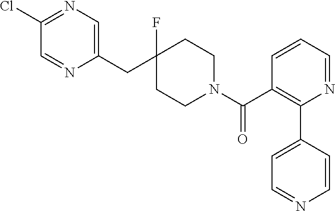 [SMILES: O=C(c1cccnc1-c1ccncc1)N1CCC(F)(Cc2cnc(Cl)cn2)CC1] |
| 97 | 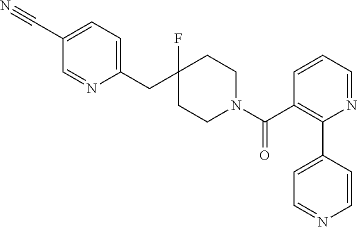 [SMILES: N#Cc1ccc(CC2(F)CCN(C(=O)c3cccnc3-c3ccncc3)CC2)nc1] |
| 98 | 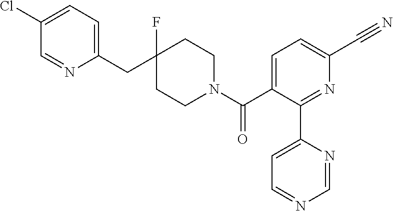 [SMILES: N#Cc1ccc(C(=O)N2CCC(F)(Cc3ccc(Cl)cn3)CC2)c(-c2ccncn2)n1] |

### Alternative Embodiments

In an alternative embodiment, compounds of Formula I may also comprise one or more isotopic substitutions. For example, hydrogen may be replaced by ²H (D or deuterium) or ³H (T or tritium); carbon may be replaced by, for example, ¹³C or ¹⁴C; oxygen may be replaced by, for example, ¹⁸O; nitrogen may be replaced by, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In another aspect, the disclosure provides a pharmaceutical composition comprising a compound of the present disclosure (e.g., a compound of Formula I or a pharmaceutically acceptable salt thereof, and a pharmaceutically acceptable excipient). The compounds of Formula I can be used in the treatment of certain disorders as described herein.

In certain embodiments, the compound of the present disclosure is provided in an effective amount in the pharmaceutical composition. In certain embodiments, the compound of the present disclosure is provided in a therapeutically effective amount. In certain embodiments, the compound of the present disclosure is provided in a prophylactically effective amount.

In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present disclosure may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, preferably from about 0.1 to about 20% by weight, preferably from about 0.1 to about 10% by weight, and more preferably from about 0.5 to about 15% by weight.

Injection dose levels range from about 0.1 mg/kg/hour to at least 20 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 5 g/day for a 40 to 80 kg human patient.

Liquid forms suitable for oral administration may include a suitable aqueous or nonaqueous vehicle with buffers, suspending and dispensing agents, colorants, flavours and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavouring agent such as peppermint, methyl salicylate, or orange flavouring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present disclosure can also be administered in sustained release forms or from sustained release drug delivery systems. A description of representative sustained release materials can be found in Remington's Pharmaceutical Sciences.

The present disclosure also relates to the pharmaceutically acceptable acid addition salt of a compound of the present disclosure. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

In another aspect, the disclosure provides a pharmaceutical composition comprising a compound of the present disclosure and a pharmaceutically acceptable excipient, e.g., a composition suitable for injection, such as for intravenous (IV) administration.

Pharmaceutically acceptable excipients include any and all diluents or other liquid vehicles, dispersion or suspension aids, surface active agents, isotonic agents, preservatives, lubricants and the like, as suited to the particular dosage form desired, e.g., injection. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

For example, injectable preparations, such as sterile injectable aqueous suspensions, can be formulated according to the known art using suitable dispersing or wetting agents and suspending agents. Exemplary excipients that can be employed include, but are not limited to, water, sterile saline or phosphate-buffered saline, or Ringer's solution.

The injectable composition can be sterilized, for example, by filtration through a bacterial-retaining filter, or by incorporating sterilizing agents in the form of sterile solid compositions which can be dissolved or dispersed in sterile water or other sterile injectable medium prior to use.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, response of the individual patient, the severity of the patient's symptoms, and the like.

The compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, pre-measured ampules or syringes of the liquid compositions. In such compositions, the compound is usually a minor component (from about 0.1% to about 50% by weight or preferably from about 1% to about 40% by weight) with the remainder being various vehicles or carriers and processing aids helpful for forming the desired dosing form.

The compounds provided herein can be administered as the sole active agent, or they can be administered in combination with other active agents. In one aspect, the present disclosure provides a combination of a compound of the present disclosure and another pharmacologically active agent. Administration in combination can proceed by any technique apparent to those of skill in the art including, for example, separate, sequential, concurrent, and alternating administration.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st}ed., Lippincott Williams & Wilkins, 2005.

In one aspect, provided is a kit comprising a composition (e.g., a solid composition) comprising a compound of Formula I.

### Methods of Use and Treatment

One aspect of the present disclosure relates to compounds that can be useful as therapeutic agents for the treatment of diseases associated with the inhibition of CYP46A1 (e.g., spasm, neurodegenerative disease, epilepsy, schizophrenia, and autism spectrum disorder). For example, in an aspect of the disclosure, provided herein is method of treating a disorder in a subject in need thereof, the method comprising administering to the subject a therapeutically effective amount of a compound or composition described herein to the subject, including the compound of Formula I as defined herein. Example of disorders that can be treated by the compounds include, but are not limited to, diseases associated with the inhibition of CYP46A1 (e.g., spasm, neurodegenerative disease, epilepsy, and schizophrenia), neurodegenerative disease, epilepsy, psychiatric disorders (e.g. schizophrenia, and autism spectrum disorder), spasm, and developmental and epileptic encephalopathies.

In some embodiments, the disease or disorder involving the inhibition of CYP46A1 is a neurodegenerative disorder. In some embodiments, the neurodegenerative disease is selected from the group consisting of Alzheimer's disease, mild cognitive impairment, Huntington's disease, Parkinson's disease, amyotrophic lateral sclerosis, traumatic brain injury, cerebral infarction, glaucoma, and multiple sclerosis. In certain embodiments, the disease or disorder involving the inhibition of CYP46A1 is epilepsy. In certain embodiments, the disease or disorder involving the inhibition of CYP46A1 is developmental and epileptic encephalopathies. In embodiments, the psychiatric disorder is selected from the group consisting of schizophrenia, delusional disorder, schizoaffective disorder, depression, and autism spectrum disorder. In embodiments, the disease or disorder involving the inhibition of CYP46A1 is spasm.

In certain embodiments, the compound is administered to the subject chronically. In certain embodiments, the compound is administered to the subject orally, subcutaneously, intramuscularly, or intravenously. In some embodiments, the compound is administered by a route of oral administration.

In one aspect, the disclosure provides a compound of the disclosure, disclosure, or pharmaceutically acceptable salt thereof, or a pharmaceutically acceptable composition comprising a compound of the disclosure, or a pharmaceutically acceptable salt thereof for use in the treatment of diseases or disorders associated with the inhibition of CYP46A1. Example of disorders that can be treated by the compounds include, but are not limited to, diseases associated with the inhibition of CYP46A1 (e.g., spasm, neurodegenerative disease, epilepsy, and schizophrenia), neurodegenerative disease, epilepsy, psychiatric disorders (e.g. schizophrenia, and autism spectrum disorder), spasm, and developmental and epileptic encephalopathies.

In one aspect, the disclosure provides the use of a compound of the disclosure, disclosure, or pharmaceutically acceptable salt thereof, for the manufacture of a medicament for the treatment of diseases or disorders associated with the inhibition of CYP46A1. Example of disorders that can be treated by the compounds include, but are not limited to, diseases associated with the inhibition of CYP46A1 (e.g., spasm, neurodegenerative disease, epilepsy, and schizophrenia), neurodegenerative disease, epilepsy, psychiatric disorders (e.g. schizophrenia, and autism spectrum disorder), spasm, and developmental and epileptic encephalopathies.

### Neurodegenerative Diseases and Disorders

A compound of the disclosure, or pharmaceutically acceptable salt thereof, or a pharmaceutically acceptable composition comprising a compound of the disclosure, or a pharmaceutically acceptable salt thereof, can be used in a method described herein, for example the treatment of neurodegenerative diseases and disorders.

The term “neurodegenerative disease” includes diseases and disorders that are associated with the progressive loss of structure or function of neurons, or death of neurons. Neurodegenerative diseases and disorders include, but are not limited to, Alzheimer's disease (including the associated symptoms of mild, moderate, or severe cognitive impairment); amyotrophic lateral sclerosis (ALS); anoxic and ischemic injuries; ataxia and convulsion (including for the treatment and prevention and prevention of seizures that are caused by schizoaffective disorder or by drugs used to treat schizophrenia); benign forgetfulness; brain edema; cerebellar ataxia including McLeod neuroacanthocytosis syndrome (MLS); closed head injury; coma; contusive injuries (e.g., spinal cord injury and head injury); dementias including multi-infarct dementia and senile dementia; disturbances of consciousness; Down syndrome; drug-induced or medication-induced Parkinsonism (such as neuroleptic-induced acute akathisia, acute dystonia, Parkinsonism, or tardive dyskinesia, neuroleptic malignant syndrome, or medication-induced postural tremor); epilepsy; fragile X syndrome; Gilles de la Tourette's syndrome; head trauma; hearing impairment and loss; Huntington's disease; Lennox syndrome; levodopa-induced dyskinesia; mental retardation; movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea), dyskinesia (including tics such as complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis); neuronal damage including ocular damage, retinopathy or macular degeneration of the eye; neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest; glaucoma (including the associated symptoms of blindness, normal intraocular pressure type field stenosis); Parkinson's disease; seizure; status epilepticus; stroke; tinnitus; tubular sclerosis, and viral infection induced neurodegeneration (e.g., caused by acquired immunodeficiency syndrome (AIDS) and encephalopathies). Neurodegenerative diseases also include, but are not limited to, neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest. Methods of treating or preventing a neurodegenerative disease also include treating or preventing loss of neuronal function characteristic of neurodegenerative disorder.

### Psychiatric Disorders

A compounds of the disclosure, or pharmaceutically acceptable salt thereof, or a pharmaceutically acceptable composition comprising a compound of the disclosure I, or a pharmaceutically acceptable salt thereof, can be used in a method described herein, for example the treatment of psychiatric disorders.

The term “psychiatric disorders” includes diseases and disorders that are associated with the clinically significant disturbance in an individual's cognition, emotion regulation, or behavior that reflects a dysfunction in the psychological, biological, or developmental processes underlying mental function. Psychiatric disorders include, but are not limited to, schizophrenia (including the associated symptoms of hallucinations, delusions, disorganized thinking, avolition, and diminished emotional expression); delusional disorder; schizoaffective disorder; dissociative identity disorder; depression, also known as depressive disorder (including the associated symptoms of persistent anxiety, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts); psychotic major depression (PMD); autism spectrum disorder; autism (including the associated symptoms of impaired social interaction, and impaired verbal and non-verbal communication); bipolar disorder (including the associated symptoms of anxiety, and mood fluctuations); and attention-deficit/hyperactivity disorder (including the associated symptoms of attention deficits, hyperactivity, and impulsiveness).

### Epilepsy

A compound of the disclosure, or pharmaceutically acceptable salt thereof, or a pharmaceutically acceptable composition comprising a compound of the disclosure, or a pharmaceutically acceptable salt thereof, can be used in a method described herein, for example the treatment of a disorder described herein such as epilepsy, developmental and epileptic encephalopathies, status epilepticus, or seizure.

Epilepsy is a syndrome of episodic brain dysfunction characterized by recurrent unpredictable, spontaneous seizures. Cerebellar dysfunction is a recognized complication of temporal lobe epilepsy and it is associated with seizure generation, motor deficits and memory impairment. Types of epilepsy can include, but are not limited to generalized epilepsy, e.g., childhood absence epilepsy, juvenile myoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### Epileptic Encephalopathies

A compound of the disclosure, or pharmaceutically acceptable salt thereof, or a pharmaceutically acceptable composition comprising a compound the disclosure, or a pharmaceutically acceptable salt thereof, can be used in a method described herein, for example the treatment of developmental and epileptic encephalopathies.

Epileptic encephalopathies are conditions in which neurologic deterioration is attributable entirely or partly to epileptic activity. It can be due to very frequent or severe seizures and/or to sub-continuous paroxysmal interictal activity. Developmental and epileptic encephalopathies represent a group of epileptic disorders that appear early in life and are characterized by pharmacoresistant generalized or focal seizures, persistent severe electroencephalography (EEG) abnormalities, and cognitive dysfunction or decline.

### Epileptogenesis

A compound of the disclosure, or pharmaceutically acceptable salt thereof, or a pharmaceutically acceptable composition comprising a compound of the disclosure, or a pharmaceutically acceptable salt thereof, can be used in a method described herein, for example the treatment of epileptogenesis.

Epileptogenesis is a gradual process by which a normal brain develops epilepsy (a chronic condition in which seizures occur). Epileptogenesis results from neuronal damage precipitated by the initial insult (e.g., status epilepticus).

### Status Epilepticus (SE)

A compound of the disclosure, or pharmaceutically acceptable salt thereof, or a pharmaceutically acceptable composition comprising a compound of the disclosure, or a pharmaceutically acceptable salt thereof, can be used in a method described herein, for example the treatment of status epilepticus (SE).

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

### Spasm

A compound of the disclosure, or pharmaceutically acceptable salt thereof, or a pharmaceutically acceptable composition comprising a compound of the disclosure, or a pharmaceutically acceptable salt thereof, can be used in a method described herein, for example the treatment of spasm.

Spasm is a disease that occurs in fits along with abnormal electric excitement of intracerebral nerve cells which includes the associated symptoms of muscle cramps, change in level of consciousness or lethargy, nausea, severe headache, sudden numbness, and vomiting. Spasm is one of the characteristic clinical findings in Alzheimer's disease.

### Combination Therapies and Treatments

When the compound of the present disclosure is applied to each of the above-mentioned diseases, it can be administered in combination with a medicament or a treatment method generally employed for the disease. Administration in combination can proceed by any technique apparent to those of skill in the art including, for example, separate, sequential, concurrent and alternating administration.

Examples of the medicament (hereinafter to be abbreviated as “concomitant drug”) to be used in combination with the compound of the present disclosure include acetylcholine esterase inhibitors (e.g., donepezil, rivastigmine, galanthamine, zanapezil etc.), antidementian agents (e.g., memantine), inhibitors of β amyloid protein production, secretion, accumulation, coagulation and/or deposition, β secretase inhibitors (e.g., 6-(4-biphenylyl)methoxy-2-[2-(N,N-dimethylamino) ethyl]tetralin, 6-(4-biphenylyl)methoxy-2-(N,N-dimethylamino)methyltetralin, 6-(4-biphenylyl)methoxy-2-(N,N-dipropylamino)methyltetralin, 2-(N,N-dimethylamino)methyl-6-(4′-methoxybiphenyl-4-yl)methoxytetralin, and 6-(4-biphenylyl)methoxy-2-[2-(N,N-diethylamino)ethyl]tetralin), γ secretase inhibitory agent, β amyloid protein coagulation inhibitory agent (e.g., PTI-00703, ALZHEMED (NC-531), PPI-368 (JP-A-11-514333), and PPI-558 (JP-A-2001-500852)), β amyloid vaccine, β amyloid degrading enzyme and the like, cerebral function activators (e.g., aniracetam, nicergoline), other therapeutic drug for Parkinson's disease (e.g., dopamine receptor agonists), a monoamine oxidase (MAO) inhibitors (e.g., deprenyl, Selgiline (selegiline), remacemide, riluzole), anticholinergic agents (e.g., trihexyphenidyl, biperiden), COMT inhibitors (e.g., entacapone)], therapeutic drug for amyotrophic lateral sclerosis (e.g., riluzole etc., neurotrophic factor), therapeutic drug for abnormal behavior, wandering and the like due to the progress of dementia (e.g., sedative drug, antianxiety drug), apoptosis inhibitors (e.g., CPI-1189, IDN-6556, CEP-1347), neuronal differentiation or regeneration promoters (e.g., leteprinim, xaliproden (SR-57746-A), SB-216763, Y-128, VX-853, prosaptide, 5,6-dimethoxy-2-[2,2,4,6,7-pentamethyl-3-(4-methylphenyl)-2,3-dihydro-1-b-enzofuran-5-yl]isoindoline and optically active forms, salts and hydrates), antidepressants (e.g., desipramine, amitriptyline, imipramine, tramadol), antiepilepsy drug (e.g., lamotrigine), antianxiety drugs (e.g., benzodiazepine), non-steroidal anti-inflammatory drugs (e.g., meloxicam, tenoxicam, indomethacin, ibuprofen, celecoxib, rofecoxib, aspirin, indomethacin), disease-modifying anti-rheumatic drugs (DMARDs), anti-cytokine drugs (e.g., TNF inhibitor, MAP kinase inhibitor), steroidal drugs (e.g., dexamethasone, hexestrol, cortisone acetate), therapeutic agents for incontinence or frequent urination (e.g., flavoxate hydrochloride, oxybutynin hydrochloride, propiverine hydrochloride), phosphodiesterase inhibitors (e.g., sildenafil (citrate)), dopamine agonists (e.g., apomorphine etc.), antiarrhythmics (e.g., mexiletine), sex hormones or derivatives thereof (e.g., progesterone, estradiol, estradiol benzoate), therapeutic agents for osteoporosis (e.g., alfacalcidol, calcitriol, elcatonin, calcitonin salmon, estriol, ipriflavone, disodium pamidronate, sodium alendronate hydrate, disodium incadronate), parathyroid hormone (PTH), calcium receptor antagonists, therapeutic drugs for insomnia (e.g., benzodiazepine medicament, non-benzodiazepine medicament, melatonin agonist), and therapeutic drugs for schizophrenia (e.g., typical antipsychotic agents such as haloperidol and the like; atypical antipsychotic agents such as clozapine, olanzapine, risperidone, aripiprazole and the like; medicament acted on metabotropic glutamate receptor or ionic channel-conjugated glutamate receptor; phosphodiesterase inhibitor).

In addition, a combined use with a transplantation method of neural stem cell or neural precursor cell prepared from embryonic stem cell or nervous tissue, or fetal neural tissue, and a combined use with a pharmaceutical agent such as an immunosuppressant after the transplantation and the like.

Furthermore, the compound of the present disclosure may be used in combination with the following concomitant drugs.

### 1. Therapeutic Agents for Diabetic Complications

For example, aldose reductase inhibitors (e.g., tolrestat, epalrestat, zenarestat, zopolrestat, minalrestat, fidarestat, CT-112), neurotrophic factor and an increasing agent thereof (e.g., NGF, NT-3, BDNF, neurotrophic factors and increasing drugs described in WO01/14372 (e.g., 4-(4-chlorophenyl)-2-(2-methyl-1-imidazolyl)-5-[3-(2-methylphenoxy)propyl-]oxazole)), nerve regeneration promoting agent (e.g., Y-128), PKC inhibitor (e.g., ruboxistaurin mesylate), AGE inhibitor (e.g., ALT946, pimagedine, pyratoxanthine, N-phenacylthiazolium bromide (ALT766), ALT-711, EXO-226, Pyridorin, pyridoxamine), active oxygen scavengers (e.g., thioctic acid), cerebral vasodilator (e.g., tiapuride, mexiletine), somatostatin receptor agonists (e.g., BIM23190), apoptosis signal regulating kinase-1 (ASK-1) inhibitor and the like can be mentioned.

### 2. Therapeutic Agent for Hyperlipidemia

For example, statin compound (e.g., pravastatin, simvastatin, lovastatin, atorvastatin, fluvastatin, rosuvastatin, pitavastatin, or a salt thereof (e.g., sodium salt, calcium salt)), squalene synthase inhibitors (e.g., lapaquistat acetate or a salt thereof), fibrate compound (e.g., bezafibrate, clofibrate, simfibrate, clinofibrate), ACAT inhibitor (e.g., Avasimibe, Eflucimibe), anion exchange resin (e.g., colestyramine), probucol, nicotinic acid drug (e.g., nicomol, niceritrol), ethyl icosapentate, phytosterol (e.g., soysterol, gamma oryzanol) and the like.

### 3. Diuretic

For example, xanthine derivative (e.g., theobromine sodium salicylate, theobromine calcium salicylate), thiazide preparation (e.g., ethiazide, cyclopenthiazide, trichloromethyazide, hydrochlorothiazide, hydroflumethiazide, benzylhydrochlorothiazide, penflutizide, polythiazide, methyclothiazide), antialdosterone preparation (e.g., spironolactone, triamterene), carbonic anhydrase inhibitors (e.g., acetazolamide), chlorobenzenesulfonamide agent (e.g., chlortalidone, mefruside, indapamide), azosemide, isosorbide, ethacrynic acid, piretanide, bumetanide, furosemide and the like.

### 4. Chemotherapeutic Agent

For example, alkylating agents (e.g., cyclophosphamide, ifosfamide), metabolic antagonists (e.g., methotrexate, 5-fluorouracil or derivative thereof), antitumor antibiotics (e.g., mitomycin, adriamycin), plant-derived antitumor agents (e.g., vincristine, vindesine, Taxol), cisplatin, carboplatin, etoposide and the like. Of these, Furtulon and NeoFurtulon, which are 5-fluorouracil derivatives, and the like are preferable.

### 5. Immunotherapeutic Agent

For example, microorganism or bacterial components (e.g., muramyl dipeptide derivative, Picibanil), polysaccharides having immunity potentiating activity (e.g., lentinan, schizophyllan, krestin), cytokines obtained by genetic engineering techniques (e.g., interferon, interleukin (IL)), colony stimulating factors (e.g., granulocyte colony stimulating factor, erythropoietin) and the like, with preference given to interleukins such as IL-1, IL-2, IL-12 and the like.

### 6. Antithrombotic Agent

For example, heparin (e.g., heparin sodium, heparin calcium, dalteparin sodium), warfarin (e.g., warfarin potassium), anti-thrombin drug (e.g., argatroban), thrombolytic agent (e.g., urokinase, tisokinase, alteplase, nateplase, monteplase, pamiteplase), platelet aggregation inhibitor (e.g., ticlopidine hydrochloride, cilostazol, ethyl icosapentate, beraprost sodium, sarpogrelate hydrochloride) and the like.

### 7. Cachexia Improving Medicament

For example, cyclooxygenase inhibitors (e.g., indomethacin etc.) [Cancer Research, Vol. 49, pages 5935-5939, 1989], progesterone derivatives (e.g., megestrol acetate) [Journal of Clinical Oncology, Vol. 12, pages 213-225, 1994], glucosteroids (e.g., dexamethasone etc.), metoclopramide agents, tetrahydrocannabinol agents (publications are all as mentioned above), fat metabolism improving agents (e.g., eicosapentanoic acid etc.) [British Journal of Cancer, Vol. 68, pages 314-318, 1993], growth hormones, IGF-1, or antibodies to a cachexia-inducing factor such as TNF-.alpha., LIF, IL-6, oncostatin M and the like.

Two or more kinds of the above-mentioned concomitant drugs may be used in combination at an appropriate ratio.

It is also possible to apply compound of the present disclosure to each of the above-mentioned diseases in combination with a biologic (e.g., antibody, vaccine preparation and the like), or as a combination therapy in combination with gene therapy method and the like.

Examples of the antibody and vaccine preparation include vaccine preparation to angiotensin II, vaccine preparation to CETP, CETP antibody, TNF.alpha. antibody and antibody to other cytokine, amyloid p vaccine preparation, type 1 diabetes vaccine (e.g., DIAPEP-277 manufactured by Peptor Ltd.), anti-HIV antibody, HIV vaccine preparation and the like, antibody or vaccine preparation to cytokine, renin-angiotensin enzyme and a product thereof, antibody or vaccine preparation to enzyme or protein involved in blood lipid metabolism, antibody or vaccine to enzyme or protein involved in blood coagulation or fibrinolytic system, antibody or vaccine preparation to protein involved in saccharometabolism or insulin resistance and the like.

In addition, a combined use with a biological preparation involved in a growth factor such as GH, IGF and the like is possible.

Examples of the gene therapy method include a treatment method using a gene relating to cytokine, renin-angiotensin enzyme and a product thereof, G protein, G protein conjugated receptor and its phosphorylation enzyme, a treatment method using a DNA decoy such as NF.kappa.B decoy and the like, a treatment method using an antisense, a treatment method using a gene relating to an enzyme or protein involved in blood lipid metabolism (e.g., gene relating to metabolism, excretion or absorption of cholesterol or triglyceride or HDL-cholesterol or blood phospholipid), a treatment method using a gene relating to an enzyme or protein involved in angiogenesis therapy targeting obstruction of peripheral vessel and the like (e.g., growth factors such as HGF, VEGF etc.), a treatment method using a gene relating to a protein involved in saccharometabolism or insulin resistance, an antisense to cytokine such as TNF and the like, and the like.

In addition, it is possible to use in combination with various organ regeneration methods such as heart regeneration, kidney regeneration, pancreas regeneration, blood vessel regeneration and the like or cell transplantation therapy utilizing bone marrow cell (myelomonocytic cell, myeloid stem cell) or an artificial organ utilizing tissue engineering (e.g., artificial blood vessel and cardiac muscle cell sheet).

The time of administration of the compound of the present disclosure and that of the concomitant drug are not limited, and they may be administered simultaneously or in a staggered manner to the administration subject. Furthermore, the compound of the present disclosure and the concomitant drug may be administered as two kinds of preparations containing each active ingredient, or a single preparation containing both active ingredients.

The dose of the concomitant drug can be appropriately determined based on the dose employed in clinical situations. The mixing ratio of the compound of the present disclosure and a concomitant drug can be appropriately determined depending on the administration subject, administration route, target disease, symptom, combination and the like. When the subject of administration is human, for example, a concomitant drug can be used in 0.01-100 parts by weight relative to 1 part by weight of the compound of the present disclosure.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art through routine optimization.

Additionally, as will be apparent to those skilled in the art, conventional protecting groups may be necessary to prevent certain functional groups from undergoing undesired reactions. The choice of a suitable protecting group for a particular functional group, as well as suitable conditions for protection and deprotection, are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative piperidines that have been listed herein. The compounds provided herein may be prepared from known or commercially available starting materials and reagents by one skilled in the art of organic synthesis. Exemplary chiral columns available for use in the separation/purification of the enantiomers/diastereomers provided herein include, but are not limited to, CHIRALPAK® AD-10, CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

¹H-NMR reported herein (e.g., for the region between δ (ppm) of about 0.5 to about 10 ppm) will be understood to be an exemplary interpretation of the NMR spectrum (e.g., exemplary peak integratations) of a compound.

Exemplary general method for LCMS/LC ELSD: 30-90AB_2 min. Lcm. (Mobile Phase: 1.5 mL/4 L TFA in water (solvent A) and 0.75 mL/4 L TFA in acetonitrile (solvent B), using the elution gradient 30%-90% (solvent B) over 0.9 minutes and holding at 90% for 0.6 minutes at a flow rate of 1.2 mL/min; Column: Xtimate C18 2.1*30 mm, 3 μm; Wavelength: UV 220 nm; Column temperature: 50° C.; MS ionization: ESI; Detector: PDA&ELSD).

### Abbreviations

ACN: acetonitrile; AcOK or KOAc: potassium acetate; AUC: area under the curve; sec-BuLi: sec-butyllithium; BSA: bis(trimethylsilyl)acetamide; BuOH: butanol; BPO: benzoyl peroxide; n-BuLi: n-butyllithium; CAN: ceric ammonium nitrate; CYP46A1: cholesterol 24-hydroxylase; DIPEA or DIEA: diisopropylethylamine; DEA: diethanolamine; DME: dimethoxyethane; DMF: dimethylformamide; DCM: dichloromethane; DMA: dimethylacetamide; DIPA: diisopropylamine; DMSO: dimethyl sulfoxide; EDCI 1-Ethyl-3-(3-dimethylaminopropyl)carbodiimide; EtOH: ethanol; EtOAc: ethyl acetate; HATU: 1-[bis(dimethylamino)methylene]-1H-1,2,3-triazolo[4,5-b]pyridinium 3-oxid hexafluorophosphate; HBSS: hank's balanced salt solution; HOBt: hydroxybenzotriazole; HSS: high strength silica; IPA: isopropyl alcohol; LC: liquid chromatography; LDA: lithium diisopropylamide; MeOD: deuterated methanol; MeCN: acetonitrile; MS: mass spectrometry; MDCK: madin-darby canine kidney cells; MDR1: multidrug resistance mutation; MeOH: methanol; NADPH: dihydronicotinamide-adenine dinucleotide phosphate; NBS: N-Bromosuccinimide; NMR: nuclear magnetic resonsnace; i-Pr₂O: diisopropyl ether; Pd₂(dba)₃: tris(dibenzylideneacetone)dipalladium(0); Pd(OAc)₂: palladium(II) Acetate; Pd(dppf)Cl₂: (1,1′-Bis(diphenylphosphino)ferrocene)palladium(II) dichloride; PE: petroleum ether; PET: polyethylene membrane; PK: pharmacokinetics; PO: per os; RFU: relative fluorescence unit; TEA: triethylamine; TFA: trifluoroacetic acid; THF: tetrahydrofuran; TQ: triple quadrupole; UPLC: ultra performance liquid chromatography.

General Synthetic Scheme

The compounds of the present invention can be prepared according to the following methods outlined in Schemes 1-3. Syntheses of specific compounds may require alterations to the reaction conditions and/or operations depicted in Schemes 1-3, which are intended to be illustrative.

As illustrated in Scheme 1, N-protected 4-piperidinones A can be treated with organometallic nucleophiles to give protected 4-piperidinols B. Deprotection to give piperidines C and subsequent amide coupling with acids D provides 4-piperidinol E. Finally, fluorination of E provides target compounds F. Alternatively, protected 4-piperidinols B can undergo fluorination followed by deprotection and subsequent amide coupling with acids D to provide target compounds F.

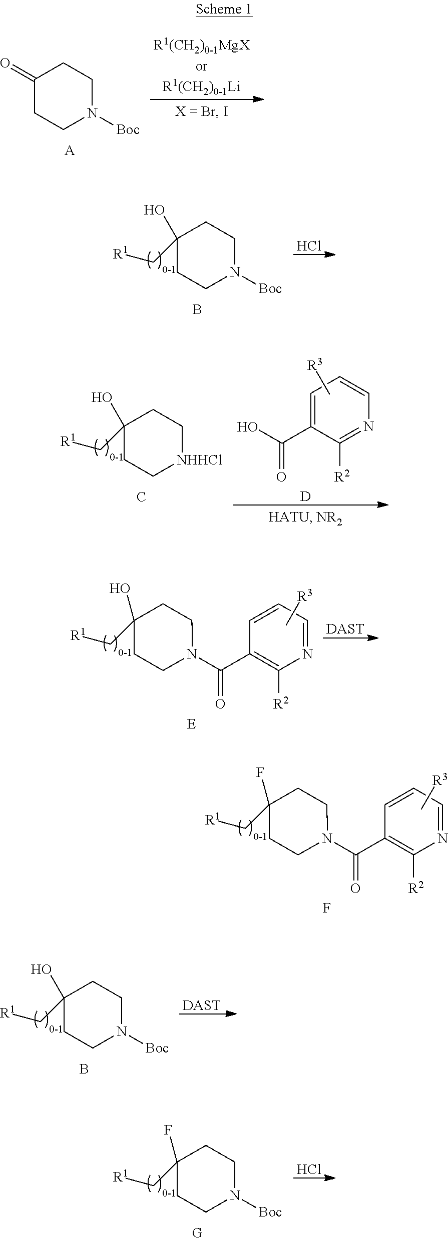

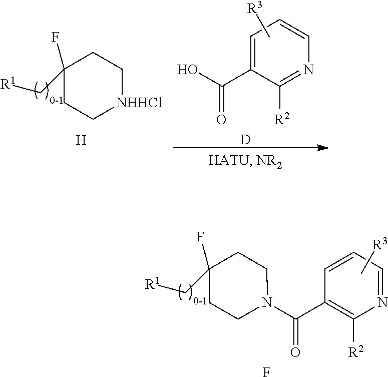

As illustrated in Scheme 2, N-protected 4-piperidinones A can be epoxidized to generate protected piperidines I. Nucleophilic epoxide opening via attack by an organometallic nucleophile affords 4-piperidinols J. Next, deprotection and subsequent amide coupling with acids D, followed by fluorination yields target compounds L. Alternatively, L may be prepared from 4-piperidinols J via a synthetic sequence involving an initial fluorination, followed by deprotection and subsequent amide coupling with acids D.

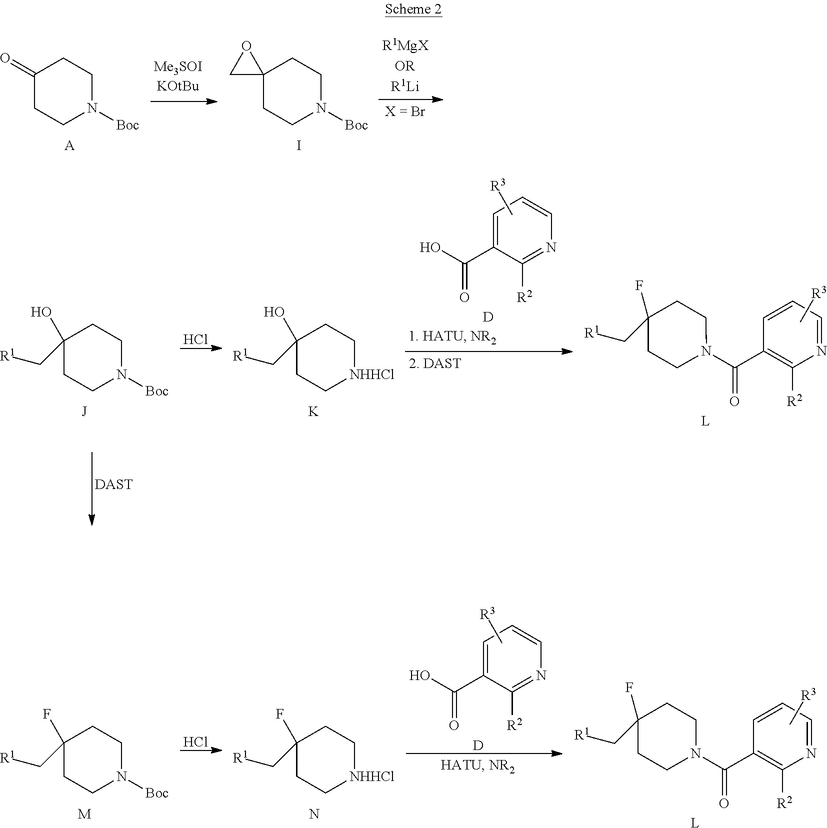

As illustrated in Scheme 3, N-protected piperide-4-carboxaldehyde A can undergo nucleophilic addition by an organometallic reagent to generate protected piperidines P. Oxidation of the pendant alcohol and subsequent fluorination then generates piperidine-derived ketones Q. Next, treatment with acid results in a net deprotection and salt formation, which affords R. Amide coupling with acids D produces piperidine-derived ketones of interest (not depicted) that can subsequently undergo mild reduction to generate target compounds S. Alternatively, ketones Q can be treated with reductant to yield piperidine-derived alcohols T. Deprotection followed by amide coupling with acids D produces target compounds S.

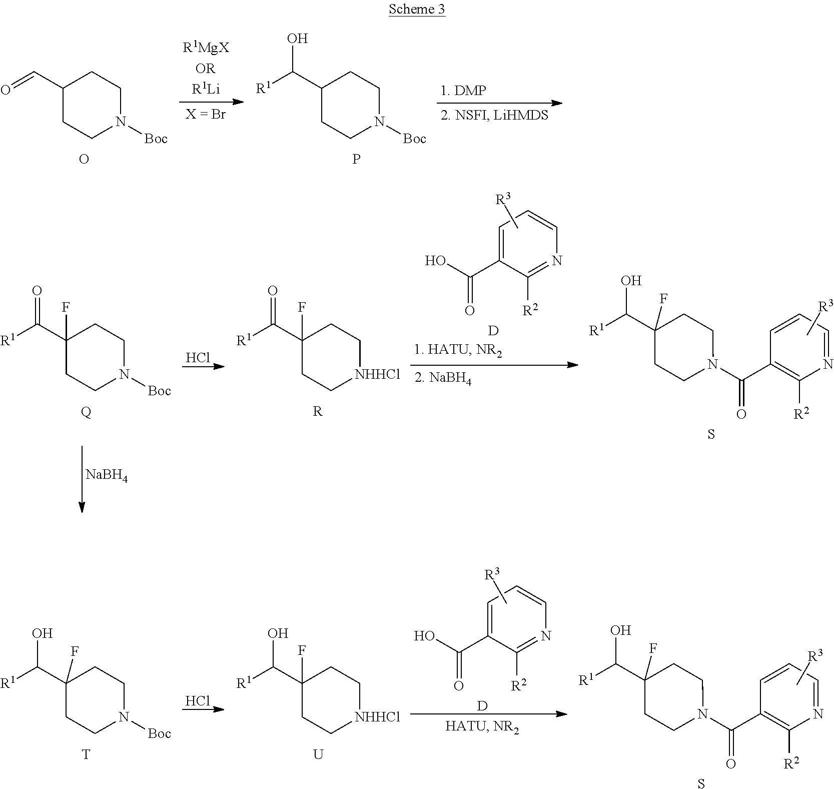

### Example 1. Synthesis of 2-(pyrimidin-4-yl)nicotinic acid (INT1)

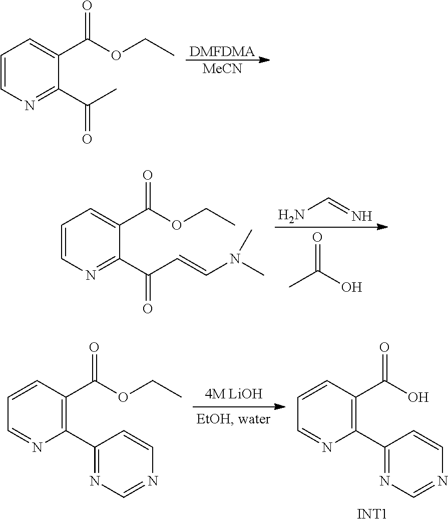

Step 1

To a solution of ethyl 2-acetylnicotinate (2.7 g, 13.9 mmol) in MeCN (27 mL) was added DMF-DMA (27 mL) in one portion at 25° C. The mixture was stirred at 85° C. for 2 h. The reaction mixture was concentrated to give ethyl (E)-2-(3-(dimethylamino)acryloyl)nicotinate (3.3 g), which was used directly in the next step.

Step 2

To a solution of ethyl (E)-2-(3-(dimethylamino)acryloyl)nicotinate (3.3 g, 13.3 mmol) and acetic acid (9.96 g, 166 mmol) in n-BuOH (30 mL) was added DIPEA (30 mL) in one portion at 25° C. The mixture was stirred at 120° C. for 40 h. The residue was poured into a mixture of water (50 mL) and saturated NaHCO₃ (18 mL). The aqueous phase was extracted with EtOAc (3×20 mL) and the combined organic extracts were washed with brine (2×20 mL), dried over anhydrous Na₂SO₄, filtered and concentrated. The residue was purified by silica gel chromatography (0˜30% EtOAc in PE) to afford ethyl-2-(pyrimidin-4-yl)nicotinate (2.3 g). ¹H NMR (400 MHz, CDCl₃) δ_{H} 9.29-9.12 (m, 1H), 9.01-8.86 (m, 1H), 8.83-8.68 (m, 1H), 8.17-7.94 (m, 2H), 7.58-7.38 (m, 1H), 4.34-4.20 (m, 2H), 1.64 (s, 3H).

Step 3

A suspension of ethyl 2-(pyrimidin-4-yl)nicotinate (2.3 g 10.0 mmol) and LiOH H₂O (629 mg, 15.0 mmol) in THE (10 mL) and MeOH (10 mL) was stirred at 25° C. for 2 hours. The mixture was concentrated, and the residue was dried in a vacuum oven at 70° C. for 2 hours and then at 100° C. for 10 minutes to give 2-(pyrimidin-4-yl)nicotinic acid (2.48 g). ¹H-NMR (400 MHz, CDCl₃) δ_{H} 9.16-9.03 (m, 1H), 8.84-8.72 (m, 1H), 8.55-8.41 (m, 1H), 7.97-7.84 (m, 1H), 7.76-7.67 (m, 1H), 7.44-7.32 (m, 1H).

### Example 2. Synthesis of lithium [2,4′-bipyridine]-3-carboxylate (INT2)

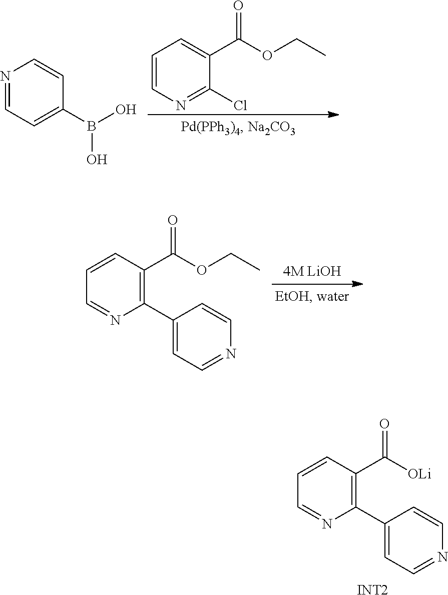

Step 1

To a mixture of pyridin-4-ylboronic acid (5 g, 0.0406 mmol), ethyl-2-chloropyridine-3-carboxylate (15 g, 81.2 mmol) and Na₂CO₃ (12.8 g, 121 mmol) in THF (20 mL) under nitrogen was added Pd(PPh₃)₄ (21.4 mg, 0.0186 mmol). The reaction mixture was stirred at 130° C. for 15 hours, cooled, and acidified (pH=2) with an aqueous solution of HCl (100 mL, 2 M). The mixture was washed with EtOAc (2×100 mL). The pH of the aqueous phase was adjusted to pH=7 and extracted with EtOAc (2×200 mL). The combined organic layers were dried over anhydrous Na₂SO₄, filtered, and concentrated to give ethyl-[2,4′-bipyridine]-3-carboxylate (7 g, 75.6%). ¹H NMR (400 MHz, CDCl₃) δ_{H} 8.85-8.79 (m, 1H), 8.72-8.66 (m, 2H), 8.21 (d, 1H), 7.49-7.42 (m, 3H), 4.25-4.15 (m, 2H), 1.12-1.07 (m, 3H).

Step 2

To a solution of ethyl-[2,4′-bipyridine]-3-carboxylate (1 g, 4.38 mmol) in THE (10 mL), MeOH (5 mL) and water (1 mL) was added LiOH H₂O (275 mg, 8.57 mmol) at 25° C. The reaction mixture was stirred at 50° C. for 3 hours and concentrated to give lithium [2,4′-bipyridine]-3-carboxylate (1.1 g). ¹H-NMR (400 MHz, DMSO-d₆) δ_{H} 8.57-8.51 (m, 2H), 8.50-8.45 (m, 1H), 7.82-7.76 (m, 2H), 7.69-7.61 (m, 1H), 7.32-7.22 (m, 1H).

### Example 3. Synthesis of (4-fluoro-4-(4-fluorobenzyl)piperidin-1-yl)(2-(pyrimidin-4-yl)pyridin-3-yl)methanone (Cmpd 1)

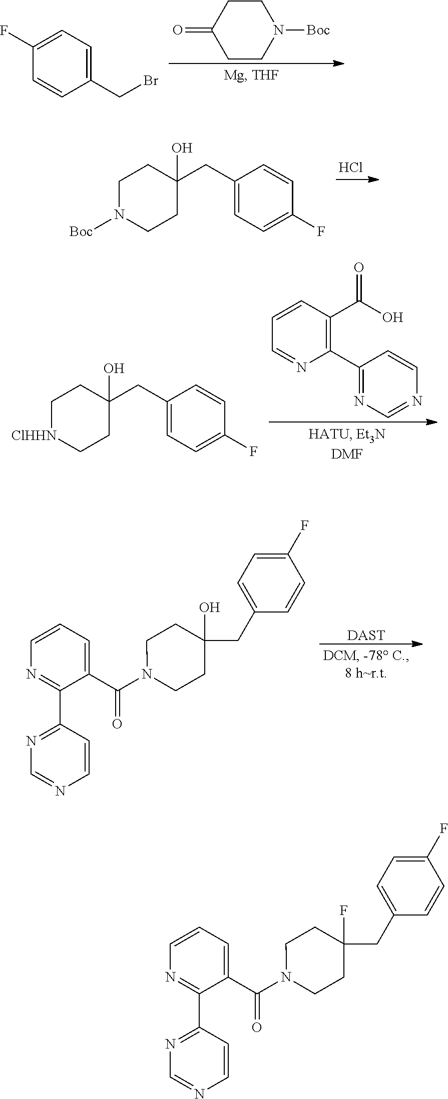

Step 1

To a suspension of magnesium (600 mg, 24.9 mmol) and iodine (63.2 mg, 0.249 mmol) in THE (20 mL) was added a solution of 1-(bromomethyl)-4-fluorobenzene (4.7 g, 24.9 mmol) in THF (10 mL) at 25° C. under nitrogen. The mixture was stirred at 25° C. for 1 hour. The mixture was cooled to 0° C., and tert-butyl 4-oxopiperidine-1-carboxylate (1 g, 5.01 mmol) was added to the mixture. The mixture was warmed to 25° C. and stirring was continued for 15 hours, and additional tert-butyl 4-oxopiperidine-1-carboxylate (700 mg) was added. The mixture was treated with water (10 mL) at 0° C. and extracted with ethyl acetate (2×50 mL). The combined organic layers were dried over anhydrous sodium sulfate, filtered and concentrated. The residue was purified by silica gel chromatography (PE/EtOAc=30/1 to 1/1) to afford tert-butyl-4-(4-fluorobenzyl)-4-hydroxypiperidine-1-carboxylate (1.5 g, 15%). ¹H-NMR (400 MHz, CDCl₃) δ_{H} 7.29-7.12 (m, 2H), 7.10-6.90 (m, 2H), 4.00-3.75 (m, 2H), 3.25-2.94 (m, 2H), 2.80-2.65 (m, 2H), 2.50-2.20 (m, 2H), 2.00-1.50 (m, 2H), 1.45 (s, 9H).

Step 2

To tert-butyl 4-(4-fluorobenzyl)-4-hydroxypiperidine-1-carboxylate (1.5 g, 4.84 mmol) was added 4M HCl in MeOH (20 mL) at 25° C. The reaction mixture was stirred at 25° C. for 1 hour, and the reaction mixture was concentrated to give 4-(4-fluorobenzyl)piperidin-4-ol hydrochloride (1.1 g, 93.2%). ¹H-NMR (400 MHz, MeOD) δ_{H} 7.41-7.18 (m, 2H), 7.15-6.90 (m, 2H), 3.30-3.10 (m, 5H), 2.90-2.65 (m, 2H), 2.05-1.88 (m, 1H), 1.86-1.72 (m, 2H), 1.71-1.60 (m, 2H).

Step 3

To a solution of 2-(pyrimidin-4-yl)nicotinic acid (200 mg, 0.994 mmol) in DMF (10 mL) was added HATU (566 mg, 1.49 mmol) at 0° C. To this mixture was added DIPEA (385 mg, 2.98 mmol) dropwise. After stirring at 0° C. for 20 minutes, 4-[(4-fluorophenyl)methyl]piperidin-4-ol hydrochloride (244 mg, 0.99 mmol) was added. The reaction was warmed to 25° C. and stirred for 30 minutes. The reaction mixture was concentrated. A second reaction was performed in parallel and the crude reaction mixtures were combined for purification. The combined reactions were purified by prep-HPLC (column: Xamide 150*30 mm 5 μm, gradient: 15-45% B (A=water, 10 mM NH₄HCO₃, B=ACN), flow rate: 25 mL/min) to give (4-(4-fluorobenzyl)-4-hydroxypiperidin-1-yl)(2-(pyrimidin-4-yl)pyridin-3-yl)methanone (325 mg, 100%). ¹H-NMR (400 MHz, CDCl₃) δ_{H} 9.19 (s, 0.5H), 8.94 (s, 0.5H), 8.90-8.81 (m, 1H), 8.76-8.72 (m, 1H), 8.29-8.18 (m, 1H), 7.75-7.60 (m, 1H), 7.52-7.38 (m, 1H), 7.18-7.12 (m, 2H), 7.05-6.98 (m, 2H), 4.70-4.46 (m, 1H), 3.64-2.88 (m, 4H), 2.77 (s, 2H), 1.95-1.61 (m, 2H), 1.41-1.24 (m, 1H), 1.21 (s, 1H). LC-ELSD/MS purity 100%, ESI cal. for C₂₂H₂₂FN₄O₂[M+H]⁺ 393, found 393.

Step 4

To a solution of (4-(4-fluorobenzyl)-4-hydroxypiperidin-1-yl)(2-(pyrimidin-4-yl)pyridin-3-yl)methanone (300 mg, 0.8 mmol) in DCM (5 mL) was added DAST (307 mg, 1.9 mmol) at −78° C. The mixture was stirred at −78° C. for 8 hours. The mixture was warmed to 10° C. and stirred for 10 hours. Saturated sodium bicarbonate solution (10 mL) was added and the aqueous layer was extracted with DCM (3×10 mL). The combined organic layers were washed with brine (10 mL), dried over anhydrous Na₂SO₄, filtered and concentrated under reduced pressure to afford 300 mg of the crude reaction mixture. The crude reaction product was purified by prep-HPLC (column: Waters Xbridge 150*25 Sum; condition: A=water (10 mM NH₄HCO₃)-B=ACN; Begin B: 33; End B: 53; Hold Time: 2; FlowRate (mL/min): 25) followed by SFC purification (column: DAICEL CHIRALPAK AD-H (250 mm*30 mm, 5 μm); condition: 0.10% NH₃H₂O EtOH; Begin B: 35; End B: 35; FlowRate (mL/min): 60) to afford (4-fluoro-4-(4-fluorobenzyl)piperidin-1-yl)(2-(pyrimidin-4-yl)pyridin-3-yl)methanone (59.0 mg, 20%). ¹H-NMR (400 MHz, DMSO-d6, t=80° C.) δ_{H} 9.07-8.90 (m, 2H), 8.76 (d, J=1.6 Hz, 1H), 8.16 (d, J=5.2 Hz, 1H), 7.80 (d, J=1.6 Hz, 1H), 7.60 (dd, J=4.8, 8.0 Hz, 1H), 7.30-7.18 (m, 2H), 7.10 (t, J=8.8 Hz, 2H), 4.40-4.27 (m, 1H), 3.35-2.96 (m, 5H), 1.90-1.50 (m, 4H). LC ELSD/MS purity ≥96%, MS ESI calcd. for C₂₂H₂₁F₂N₄O [M+H]⁺ 395, found 395. ¹⁹F-NMR (376.5 MHz, CDCl₃) δ_{F} −115.7, −161.1.

### Example 4. Synthesis of (4-fluoro-4-((tetrahydro-2H-pyran-4-yl)methyl)piperidin-1-yl)(2-(pyrimidin-4-yl)pyridin-3-yl)methanone (Cmpd 2)

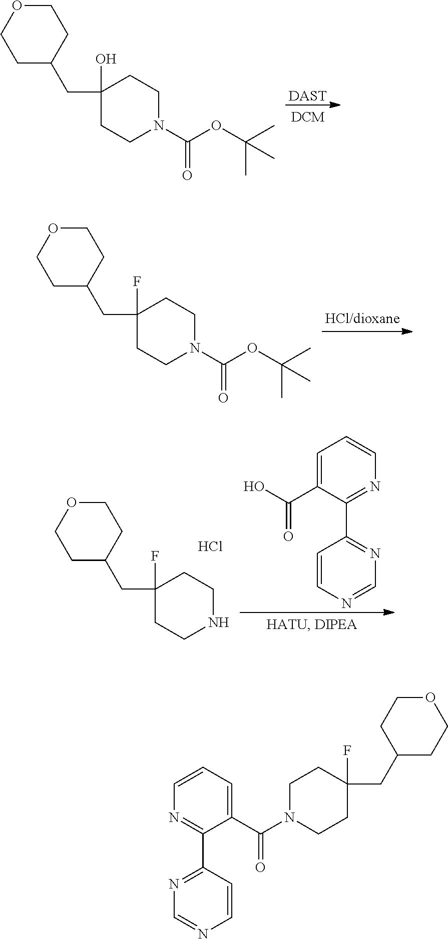

Step 1

To a solution of tert-butyl 4-hydroxy-4-((tetrahydro-2H-pyran-4-yl)methyl)piperidine-1-carboxylate (300 mg, 1.00 mmol) in DCM (4 mL) was added DAST (322 mg, 2.00 mmol) at −70° C. After stirring at −70° C. for 1 hour, the mixture was poured into sat. NaHCO₃ (10 mL) and extracted with DCM (2×20 mL). The combined organic layers were washed with brine (10 mL), dried over Na₂SO₄, filtered and concentrated. The residue was purified by silica gel chromatography (PE: EtOAc 10% to 15% to 20%) to give tert-butyl 4-fluoro-4-((tetrahydro-2H-pyran-4-yl)methyl)piperidine-1-carboxylate (180 mg, 59.8%). ¹H-NMR (400 MHz, CDCl₃)^{6H}3.94-3.90 (m, 4H), 3.42-3.36 (m, 2H), 3.09-3.03 (m, 2H), 1.67-1.64 (m, 3H), 1.57-1.51 (m, 2H), 1.50 (s, 2H), 1.45-1.37 (m, 2H), 1.36 (s, 9H), 1.34-1.29 (m, 2H).

Step 2

To a solution of tert-butyl 4-fluoro-4-((tetrahydro-2H-pyran-4-yl)methyl)piperidine-1-carboxylate (230 mg, 0.76 mmol) in MeOH (3 mL) was added 4M HCl/Dioxane (222 mg, 1.52 mL) at 25° C. The mixture was stirred at 25° C. for 2 hours under nitrogen and concentrated to give 4-fluoro-4-((tetrahydro-2H-pyran-4-yl)methyl)piperidine hydrochloride (210 mg). ¹H NMR (400 MHz, CDCl₃) δ_{H} 3.92 (br dd, J=3.8, 10.8 Hz, 2H), 3.62 (s, 1H), 3.51-3.40 (m, 2H), 3.35 (br s, 2H), 3.27-3.16 (m, 2H), 2.18 (br dd, J=7.8, 13.3 Hz, 2H), 1.99-1.79 (m, 3H), 1.77-1.69 (m, 3H), 1.46-1.26 (m, 2H).

Step 3

To a solution of 4-fluoro-4-((tetrahydro-2H-pyran-4-yl)methyl)piperidine hydrochloride (95 mg, 0.40 mmol) in DMF (1 mL) was added HATU (227 mg, 0.60 mmol) and DIPEA (0.206 mL, 1.19 mmol) at 25° C. After stirring for 30 minutes at 25° C., 2-(pyrimidin-4-yl)nicotinic acid (80.3 mg, 0.40 mmol) was added. The mixture was stirred for 10 hours at 25° C., concentrated and purified by prep-HPLC (Column: Phenomenex Gemini-NX 150*30 mm*5 um; Condition: A=water (0.04% NH₃H₂O+10 mM NH₄HCO₃)-B=ACN; Begin B:22, End B:52; Gradient Time(min):3; 100% B Hold Time(min):2; FlowRate(ml/min):30; Injections:7) to afford the product (4-fluoro-4-((tetrahydro-2H-pyran-4-yl)methyl)piperidin-1-yl)(2-(pyrimidin-4-yl)pyridin-3-yl)methanone (26.4 mg, 17.2%). ¹H-NMR (400 MHz, CDCl₃) δ_{H} 9.25-9.06 (m, 1H), 8.89 (br d, J=4.5 Hz, 1H), 8.76 (dd, J=1.6, 4.6 Hz, 1H), 8.25 (br d, J=4.8 Hz, 1H), 7.69 (br d, J=6.5 Hz, 1H), 7.47 (dd, J=4.8, 7.5 Hz, 1H), 4.78-4.46 (m, 1H), 3.94 (br d, J=11.3 Hz, 2H), 3.47-3.35 (m, 3H), 3.28-3.09 (m, 2H), 2.18-1.99 (m, 1H), 1.98-1.66 (m, 5H), 1.55 (br d, J=5.3 Hz, 2H), 1.45-1.33 (m, 2H), 1.29-0.94 (m, 1H). LC-MS: purity 100%, MS ESI calcd. for C₂₁H₂₅FN₄O₂ [M+H]⁺ 385.3, found 385.3. ¹⁹F-NMR (376.5 MHz, CDCl₃) δ_{F} −1⁶².

### Example 5. Synthesis of [2,4′-bipyridin]-3-yl(4-fluoro-4-((tetrahydro-2H-pyran-4-yl)methyl)piperidin-1-yl)methanone Cmpd 3)

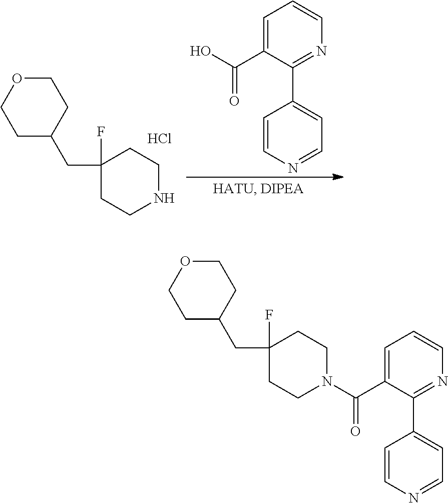 [SMILES: FC1(CC2CCOCC2)CCNCC1 ; Cl ; O=C(O)c1cccnc1-c1ccncc1 ; O=C(c1cccnc1-c1ccncc1)N1CCC(F)(CC2CCOCC2)CC1]

To a solution of 4-fluoro-4-((tetrahydro-2H-pyran-4-yl)methyl)piperidine hydrochloride (95 mg, 0.40 mmol) in DMF (1 mL) was added HATU (227 mg, 0.60 mmol) and DIPEA (0.206 mL, 1.19 mmol) at 25° C. After stirring for 30 minutes at 25° C., [2,4′-bipyridine]-3-carboxylic acid (79.9 mg, 0.4 mmol) was added to the solution. The mixture was stirred for 10 hours at 25° C., concentrated and purified by prep-HPLC (Column: Phenomenex Gemini-NX 150*30 mm*5 um; Condition: A=water (0.04% NH₃H₂O+10 mM NH₄HCO₃)-B=ACN; Begin B:22, End B:52; Gradient Time(min):3; 100% B Hold Time(min):2; FlowRate(ml/min):30; Injections:7) to afford the product [2,4′-bipyridin]-3-yl(4-fluoro-4-((tetrahydro-2H-pyran-4-yl)methyl)piperidin-1-yl)methanone (31.9 mg, 20.8%). ¹H-NMR (400 MHz, CDCl₃) δ_{H} 8.94-8.56 (m, 3H), 7.89-7.55 (m, 3H), 7.45 (br dd, J=4.8, 7.5 Hz, 1H), 4.75-4.48 (m, 1H), 3.90 (br dd, J=3.3, 11.3 Hz, 2H), 3.36 (br t, J=11.8 Hz, 2H), 3.17-2.60 (m, 3H), 2.05-1.69 (m, 1H), 1.58-1.33 (m, 5H), 1.32-0.73 (m, 5H). LC-MS: purity 100%, MS ESI calcd. for C₂₂H₂₆FN₃O₂[M+H]⁺ 384.3, found 384.3. ¹⁹F-NMR (376.5 MHz, CDCl₃) δ_{F} −162.

### Example 6. Synthesis of (4-fluoro-4-(4-(trifluoromethyl)benzyl)piperidin-1-yl)(2-(pyrimidin-4-yl)pyridin-3-yl)methanone (Cmpd 4)

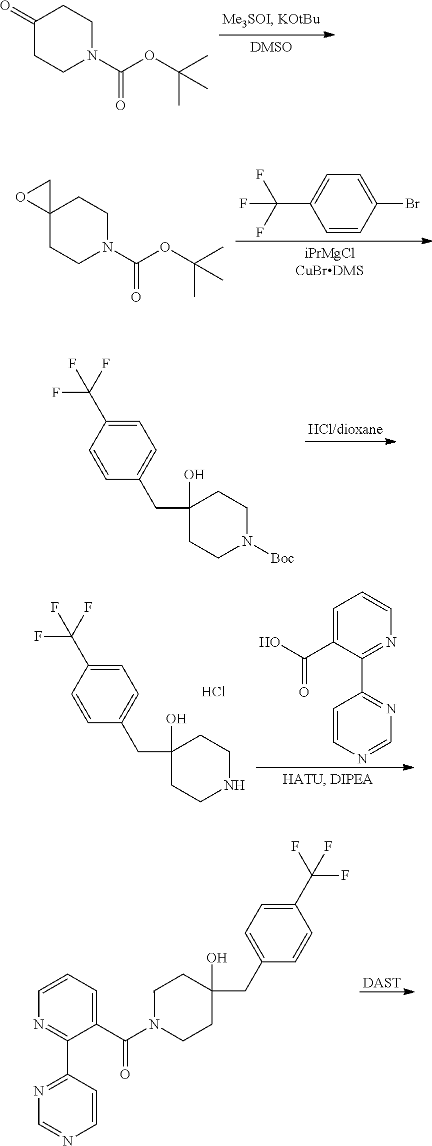

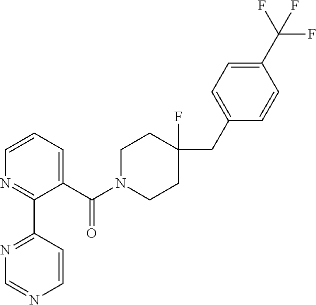 [SMILES: O=C(c1cccnc1-c1ccncn1)N1CCC(F)(Cc2ccc(C(F)(F)F)cc2)CC1]

Step 1

To a solution of Me₃SOI (12.1 g, 55.1 mmol) in DMSO (100 mL) was added t-BuOK (6.74 g, 60.1 mmol) at 0° C., and the mixture was stirred at 20° C. for 1 hour. The mixture was cooled to 0° C., and a solution of tert-butyl 4-oxopiperidine-1-carboxylate (10 g, 50.1 mmol) in DMSO (50 mL) was slowly added. The mixture was stirred at 0° C. for 3 hours, poured into an aqueous solution of NH₄Cl (200 mL) and extracted with EtOAc (3×200 mL). The combined organic layers were washed with brine (2×100 mL), dried over Na₂SO₄, filtered, and concentrated. The residue was applied to a silica gel pad and eluted with PE:EtOAc (3:1, 3×150 mL). The filtrate was concentrated to give tert-butyl-1-oxa-6-azaspiro[2.5]octane-6-carboxylate (9 g, 85%), which was used directly in the next reaction.

Step 2

To a solution of [4-(trifluoromethyl)phenyl]magnesium chloride (9.5 mmol) in THE (20 mL) was slowly added tert-butyl 1-oxa-6-azaspiro[2.5]octane-6-carboxylate (1.5 g, 7.03 mmol) in THE (10 mL) at 25° C., and the mixture was stirred 25° C. for 2 hours. The mixture was poured into an aqueous solution of NH₄Cl (20 mL) and extracted with EtOAc (3×50 mL). The combined organic layers were washed with brine (20 mL), dried over Na₂SO₄, filtered, and concentrated. The crude product was purified by silica gel chromatography (0˜30% of EtOAc in PE) to give tert-butyl 4-hydroxy-4-(4-(trifluoromethyl)benzyl)piperidine-1-carboxylate (900 mg, 36%), which was used directly in the next reaction.

Step 3

To a mixture of tert-butyl 4-hydroxy-4-(4-(trifluoromethyl)benzyl)piperidine-1-carboxylate (900 mg, 2.50 mmol) in dioxane (5 mL) was added HCl/dioxane (4 M, 5 mL, 20 mmol), and the mixture was stirred at 25° C. for 2 hours. The reaction mixture was concentrated to give 4-(4-(trifluoromethyl)benzyl)piperidin-4-ol hydrochloride (600 mg, crude), which was used directly in the next reaction.

Step 4

To a solution of 4-(4-(trifluoromethyl)benzyl)piperidin-4-ol hydrochloride (300 mg, 1.01 mmol) and 2-(pyrimidin-4-yl)nicotinic acid (243 mg, 1.21 mmol) in DMF (5 mL) was added HATU (640 mg, 1.51 mmol) and DIPEA (651 mg, 5.05 mmol). After stirring at 25° C. for 16 hours, the mixture was diluted with H₂O (10 mL) and extracted with EtOAc (2×50 mL). The combined organic phase was washed sequentially with H₂O (2×30 mL) and brine (2×30 mL), dried over Na₂SO₄, filtered and concentrated to give the crude product (300 mg, crude). The crude product (100 mg) was purified by prep-HPLC (Column: Phenomenex Gemini-NX 150*30 mm*5 μm; Condition: A=water (0.04% NH₃H₂O+10 mM NH₄HCO₃)-B=ACN; Begin B: 29%; End 59%) to give (4-hydroxy-4-(4-(trifluoromethyl)benzyl)piperidin-1-yl)(2-(pyrimidin-4-yl)pyridin-3-yl)methanone (34.6 mg, 11%). ¹H-NMR (400 MHz, CDCl₃) δ_{H} 9.24-9.01 (m, 1H), 8.93-8.81 (m, 1H), 8.79-8.70 (m, 1H), 8.30-8.21 (m, 1H), 7.75-7.65 (m, 1H), 7.64-7.54 (m, 2H), 7.50-7.42 (m, 1H), 7.35-7.27 (m, 2H), 4.65-4.45 (m, 1H), 3.50-3.33 (m, 1H), 3.28-3.10 (m, 1H), 2.92-2.77 (m, 2H), 2.00-1.85 (m, 1H), 1.82-1.65 (m, 2H), 1.47-1.16 (m, 2H). ¹⁹F NMR (376.5 MHz, CDCl3) δ_{F} −62.501. LCMS: purity 99%, MS ESI calcd. for C₂₃H₂₁F₃N₄O₂[M+H]⁺ 443.2, found 443.0.

Step 5

To a solution of (4-hydroxy-4-(4-(trifluoromethyl)benzyl)piperidin-1-yl)(2-(pyrimidin-4-yl)pyridin-3-yl)methanone (60 mg, 0.14 mmol) in DCM (2 mL) was added DAST (26.2 mg, 0.20 mmol) at −78° C. After stirring at −78° C. for 2 hours, the mixture was poured into a saturated aqueous solution of NaHCO₃ (10 mL) and extracted with DCM (2×20 mL). The combined organic layers were washed with brine (10 mL), dried over Na₂SO₄, filtered and concentrated. The residue was purified by silica gel chromatography (MeOH in EtOAc=5% to 10%) to give 40 mg of product (impure), which was triturated from isopropyl ether (2 mL) at 25° C. to give (4-fluoro-4-(4-(trifluoromethyl)benzyl)piperidin-1-yl)(2-(pyrimidin-4-yl)pyridin-3-yl)methanone (7.8 mg, 12%). ¹H-NMR (400 MHz, CDCl₃) δ_{H} 9.19 (s, 1H), 8.84-8.98 (m, 2H), 8.74-8.72 (m, 1H), 8.27-8.21 (m, 1H), 7.72-7.64 (m, 2H), 7.57 (d, J=7.60 Hz, 2H), 7.44-7.41 (m, 1H), 7.32-7.29 (m, 2H), 5.22-5.44 (m, 1H), 4.50-4.76 (m, 1H), 3.36-3.49 (m, 1H), 2.91-3.27 (m, 4H), 2.22 (t, J=7.60 Hz, 1H), 1.62-2.03 (m, 4H), 1.35-1.24 (m, 4H), 0.84-0.91 (m, 1H). LCMS: purity 99%, MS ESI calcd. for C₂₃H₂₀N₄F₄O [M+H]⁺ 445.2, found 445.0. ¹⁹F-NMR (376.5 MHz, CDCl₃) δ_{F} −62.5, −162.

### Example 7. Synthesis of (4-fluoro-4-(pyridin-2-ylmethyl)piperidin-1-yl)(2-(pyrimidin-4-yl)pyridin-3-yl)methanone (Cmpd 5)

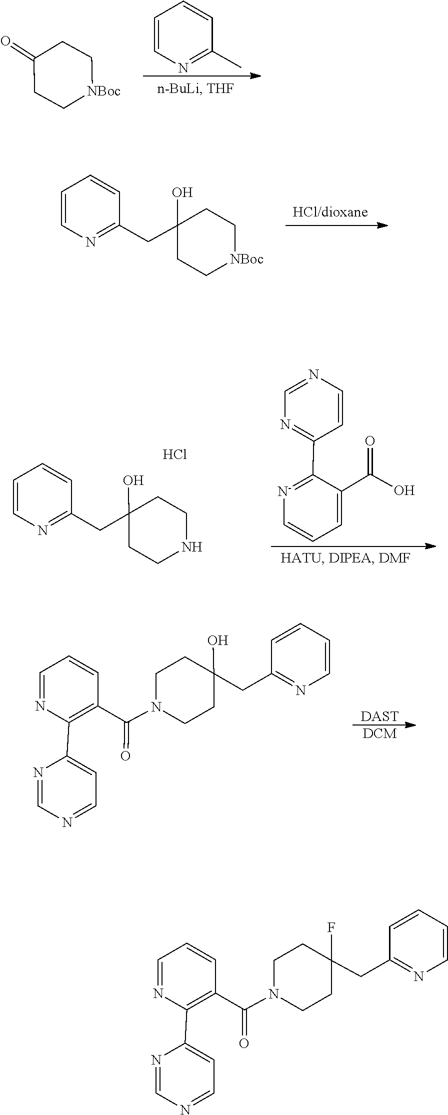

Step 1

To a solution of 2-methylpyridine (2 g, 21.4 mmol) in THE (20 mL) was added dropwise n-BuLi (10.2 mL, 2.5 M solution in Hexane, 25.6 mmol) at −78° C. under nitrogen. The reaction mixture was stirred for 30 minutes at −78° C., and tert-butyl 4-oxopiperidine-1-carboxylate (4.26 g, 21.4 mmol) in THF (10 mL) was added over 15 minutes. The mixture allowed to warm to 25° C. and was stirred for 16 hours. Saturated aqueous NH₄Cl solution (100 mL) was added, and the mixture was extracted with EtOAc (3×100 mL). The combined organic layers were washed with brine (2×100 mL), dried over Na₂SO₄, filtered and concentrated to give tert-butyl 4-hydroxy-4-(pyridin-2-ylmethyl)piperidine-1-carboxylate (6.06 g, crude). ¹H-NMR (400 MHz, CDCl₃) δ_{H} 8.48-8.46 (m, 1H), 7.64-7.60 (m, 1H), 7.26-7.08 (m, 2H), 3.77-3.69 (m, 2H), 3.22-3.20 (m, 2H), 2.86-2.84 (m, 2H), 1.48-1.43 (m, 12H).

Step 2

To a mixture of tert-butyl 4-hydroxy-4-(pyridin-2-ylmethyl)piperidine-1-carboxylate (2 g, 6.84 mmol) in dioxane (30 mL) was added HCl/dioxane (4 M, 30 mL, 120 mmol). The mixture was stirred at 20° C. for 16 h. The reaction mixture was filtered, and the filter cake washed with 10 mL of EtOAc, and dried to give 4-(pyridin-2-ylmethyl)piperidin-4-ol hydrochloride (1.5 g, crude). ¹H-NMR (400 MHz, CDCl₃) δ_{H} 8.81-8.80 (m, 1H), 8.62-8.57 (m, 1H), 8.08-8.02 (m, 2H), 3.32-3.24 (m, 5H), 2.09-2.03 (m, 2H), 1.78-1.74 (m, 2H).

Step 3

To a solution of 2-(pyrimidin-4-yl)nicotinic acid (600 mg, 2.98 mmol), 4-[(pyridin-2-yl)methyl]piperidin-4-ol hydrochloride (612 mg, 2.68 mmol) and HATU (1.69 g, 4.47 mmol) in DMF (15 mL) was added DIPEA (1.92 g, 14.9 mmol). The mixture was stirred at 20° C. for 2 h. The reaction mixture was poured into H₂O (50 mL) and the aqueous layer was extracted with EtOAc (2×100 mL). The combined organic layers were washed with brine (100 mL), dried over anhydrous Na₂SO₄, and evaporated under reduced pressure to give crude product, which was purified by prep-HPLC (Column: Phenomenex Gemini-NX 150*30 mm*5 um; Condition: A=water (0.04% NH₃H₂O+10 mM NH₄HCO₃)-B=ACN; Begin B: 10%; End 40%) to give product (200 mg, impure), which was further purified by prep-TLC to give 4-[(pyridin-2-yl)methyl]-1-[2-(pyrimidin-4-yl)pyridine-3-carbonyl]piperidin-4-ol (57 mg, 6%). ¹H-NMR (400 MHz, CDCl₃) δ_{H} 9.22-918 (m, 1H), 8.86-8.83 (m, 1H), 8.72-8.71 (m, 1H), 8.47-8.46 (m, 1H), 7.69-7.67 (m, 1H), 7.63-7.62 (m, 1H), 7.44-7.43 (m, 1H), 7.18-7.17 (m, 1H), 7.12-7.10 (m, 2H), 4.52-4.46 (m, 1H) 3.52-3.49 (m, 1H), 3.38-3.35 (m, 2H), 3.14-3.10 (m, 1H), 2.91-2.88 (m, 1H), 1.74-1.71 (m, 2H), 1.51-1.50 (m, 2H), 1.34-1.31 (m, 1H). HPLC purity 99%, MS ESI calcd for C₂₁H₂₂N₅O₂ [M+H]⁺ 376.3, found 376.3.

Step 4

To a mixture of 4-[(pyridin-2-yl)methyl]-1-[2-(pyrimidin-4-yl)pyridine-3-carbonyl]piperidin-4-ol (90 mg, 0.2397 mmol) in DCM (2 mL) was added DAST (77.2 mg, 0.4794 mmol) at 0° C. After stirring at 20° C. for 16 hours, the reaction mixture was concentrated. The residue was purified by prep-HPLC (Column: YMC-Triart Prep C18 150*40 mm*7 um; Condition: A=water (0.04% NH₃H₂O+10 mM NH₄HCO₃)-B=ACN; Begin B: 42%; End 42%) to give (4-fluoro-4-(pyridin-2-ylmethyl)piperidin-1-yl)(2-(pyrimidin-4-yl)pyridin-3-yl)methanone (2.2 mg 2.4%). ¹H-NMR (400 MHz, CDCl₃) δ_{H} 9.24-8.53 (m, 4H), 8.28-8.17 (m, 1H), 7.65 (br d, J=7.8 Hz, 2H), 7.44 (br d, J=4.5 Hz, 1H), 7.18 (br d, J=5.0 Hz, 2H), 4.82-4.38 (m, 1H), 3.43 (br d, J=8.5 Hz, 1H), 3.26-3.08 (m, 4H), 2.00 (br d, J=15.8 Hz, 2H), 1.69 (br s, 2H). LC-ELSD/MS purity 96%, MS ESI calcd for C₂₁H₂₀FN₅O [M+H]⁺ 378.3, found 378.3. ¹⁹F-NMR (376.5 MHz, CDCl₃) δ_{F} −158.

### Example 8. Synthesis of 4-((4-fluoro-1-(2-(pyrimidin-4-yl)nicotinoyl)piperidin-4-yl)methyl)benzonitrile (Cmpd 6)

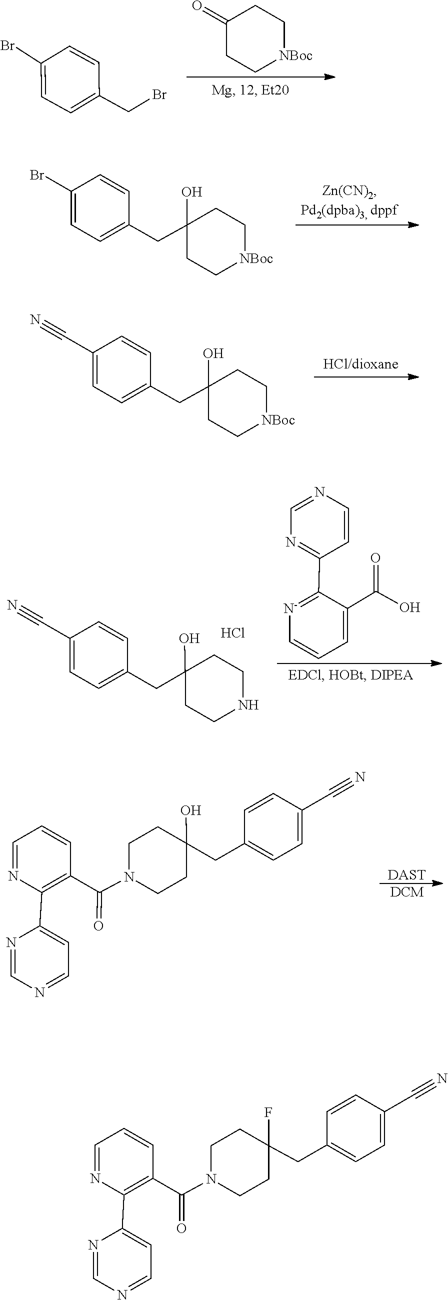

Step 1

To a suspension of Mg (3.90 g, 150 mmol) and I₂ (100 mg, 0.391 mmol) in Et₂O (60 mL) was added 1-bromo-4-(bromomethyl)benzene (9.37 g, 37.5 mmol) in Et₂O (10 mL) at 20° C. under nitrogen. The reaction mixture was stirred at 40° C. for 30 minutes, and tert-butyl 4-oxopiperidine-1-carboxylate (3 g, 15.0 mmol) in diethyl ether (10 mL) was slowly added. The mixture was heated at 40° C. for 1 hour. The mixture was cooled and added to a saturated aqueous solution of NH₄Cl (30 mL), and the mixture was extracted with EtOAc (20 mL×3). The combined organic layers were washed with brine (50 mL), dried over Na₂SO₄, filtered and concentrated. The residue was purified by silica gel chromatography (0˜10% of EtOAc in PE) to afford tert-butyl 4-(4-bromobenzyl)-4-hydroxypiperidine-1-carboxylate (4 g, 72.0%). ¹H-NMR (400 MHz, CDCl₃) δ_{H} 7.44 (d, J=8.4 Hz, 2H), 7.07 (d, J=8.4 Hz, 2H), 3.93-3.76 (m, 2H), 3.16-2.99 (m, 2H), 2.71 (s, 2H), 1.62-1.55 (m, 2H), 1.45 (s, 11H).

Step 2

To a solution of tert-butyl 4-(4-bromobenzyl)-4-hydroxypiperidine-1-carboxylate (4 g, 10.8 mmol) in DMF (40 mL) was added Zn(CN)₂ (8.76 g, 75.6 mmol) at 20° C. under nitrogen. To this mixture was added Pd₂(dba)₃ (1.67 g, 1.83 mmol) and dppf (2.81 g, 5.07 mmol). The mixture was stirred at 130° C. for 16 hours. Ice-water (70 mL) was added, and the mixture was extracted with EtOAc (70 mL×2). The organic phase was dried over Na₂SO₄, concentrated and purified by silica gel chromatography (0˜90% of EtOAc in PE) to give tert-butyl 4-(4-cyanobenzyl)-4-hydroxypiperidine-1-carboxylate (4 g, impure), which was used without further purification in the next step. ¹H-NMR (400 MHz, CDCl₃) δ_{H} 7.65-7.58 (m, 2H), 7.34-7.30 (m, 2H), 3.94-3.77 (m, 2H), 3.16-2.99 (m, 2H), 2.82 (s, 2H), 1.66-1.56 (m, 2H), 1.50-1.47 (m, 2H), 1.45 (s, 9H).

Step 3

To a solution of tert-butyl 4-(4-cyanobenzyl)-4-hydroxypiperidine-1-carboxylate (1 g, 3.16 mmol) in dioxane (2 mL) was added HCl/Dioxane (10 mL, 4M, 40.0 mmol) at 25° C. The mixture was stirred at 25° C. for 12 hours under nitrogen. The mixture was concentrated to give 4-((4-hydroxypiperidin-4-yl)methyl)benzonitrile hydrochloride (800 mg, crude), which was used directly in the next step.

Step 4

A solution of 2-(pyrimidin-4-yl)nicotinic acid (762 mg, 3.78 mmol), HOBt (853 mg, 6.32 mmol), EDCI (1.20 g, 6.32 mmol), DIPEA (1.64 mL, 9.48 mmol) and 4-((4-hydroxypiperidin-4-yl)methyl)benzonitrile hydrochloride (800 mg, 3.16 mmol) in DCM (2 mL) was stirred at 0° C. for 2 hours. The mixture was filtered and purified by prep-HPLC (Column: Xtimate C18 150*40 mm*5 um; Condition: A=water (10 mM NH₄HCO₃)-B=ACN; Begin B: 25; End B: 35; Gradient Time (min):8; 100% B Hold Time (min): 2) to afford 4-((4-hydroxy-1-(2-(pyrimidin-4-yl)nicotinoyl)piperidin-4-yl)methyl)benzonitrile (250 mg, 19.8%). ¹H-NMR (400 MHz, CDCl₃) δ_{H} 9.18 (s, 0.5H), 9.03 (s, 1H), 8.93-8.81 (m, 0.5H), 8.78-8.71 (m, 1H), 8.25 (d, J=4.4 Hz, 1H), 7.74-7.64 (m, 1H), 7.64-7.59 (m, 2H), 7.50-7.41 (m, 1H), 7.35-7.28 (m, 2H), 4.66-4.46 (m, 1H), 3.46-3.32 (m, 1H), 3.30-3.12 (m, 2H), 2.91-2.78 (m, 2H), 1.92 (br s, 0.5H), 1.78-1.63 (m, 1.5H), 1.42 (s, 0.5H), 1.30-1.19 (m, 1.5H). LCMS purity 99%, MS ESI calcd. for C₂₃H₂₂N₅O₂ [M+H]⁺ 400, found 400.

Step 5

To a mixture of 4-((4-hydroxy-1-(2-(pyrimidin-4-yl)nicotinoyl)piperidin-4-yl)methyl)benzonitrile (120 mg, 0.300 mmol) in DCM (4 mL) was added DAST (28.0 mg, 0.90 mmol) at 0° C. The mixture was stirred at 0° C. for 2 hours. The reaction mixture was concentrated and the residue was purified by SFC (Column: DAICEL CHIRALPAK AD (250 mm*30 mm, 10 um); Condition: water (0.1% NH₃H₂O IPA; Begin B: 40%; End 40%) to give 4-((4-fluoro-1-(2-(pyrimidin-4-yl)nicotinoyl)piperidin-4-yl)methyl)benzonitrile (17.6 mg, 14%).

¹H-NMR (400 MHz, CDCl₃) δ_{H} 9.18 (s, 0.3H), 8.95 (s, 0.6H), 8.92-8.83 (m, 1H), 8.79-8.71 (m, 1H), 8.30-8.21 (m, 1H), 7.72-7.64 (m, 1H), 7.63-7.57 (m, 2H), 7.48-7.42 (m, 1H), 7.35-7.28 (m, 2H), 4.76-4.53 (m, 1H), 3.49-3.32 (m, 1H), 3.29-3.05 (m, 2H), 3.05-2.88 (m, 2H), 2.05-1.59 (m, 3.5H), 1.52-1.46 (m, 0.5H). LCMS purity 98%, MS ESI calcd. for C₂₃H₂₁FN₅O [M+H]⁺ 402, found 402.

### Example 9. Synthesis of (4-((5-cyclopropylpyridin-2-yl)methyl)-4-fluoropiperidin-1-yl)(2-(pyrimidin-4-yl)pyridin-3-yl)methanone (Cmpd 7)

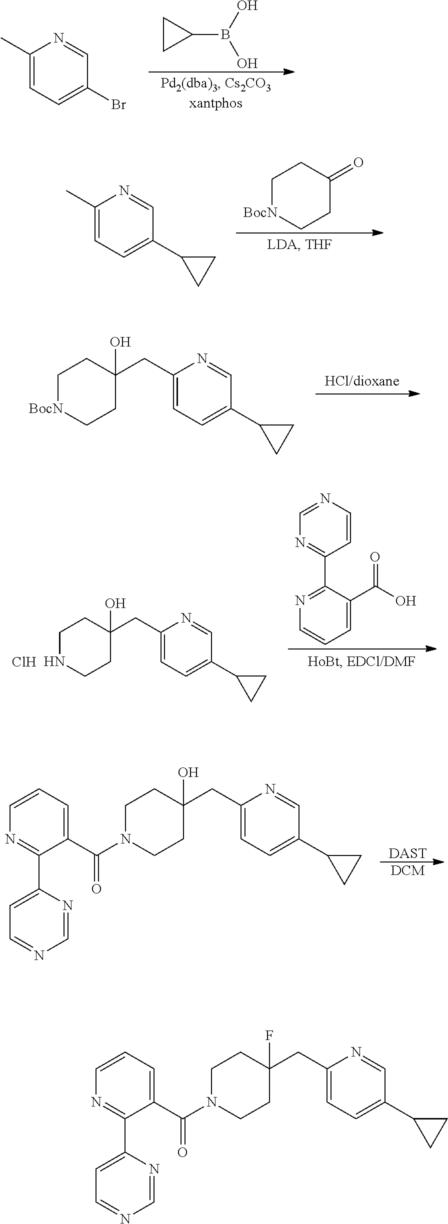

Step 1

A mixture of 5-bromo-2-methylpyridine (2.0 g, 11.6 mmol), cyclopropyl boronic acid (1.19 g, 13.9 mmol), Xantphos (1.34 g, 2.76 mmol), Pd₂(dba)₃ (1.26 g, 1.38 mmol) and Cs₂CO₃ (4.80 g, 34.8 mmol) in dioxane (20 mL) was stirred at 100° C. for 16 hours under nitrogen. The reaction mixture was poured into H₂O (50 mL) and the aqueous layer was extracted with EtOAc (2×20 mL). The combined organic layers were washed with brine (30 mL), dried over anhydrous Na₂SO₄ and concentrated. The crude product was purified by silica gel chromatography (5˜30% of EtOAc in PE) to give 5-cyclopropyl-2-methylpyridine (600 mg, 38.9%). ¹H-NMR (400 MHz, CDCl₃) δ_{H} 8.31 (d, J=2.0 Hz, 1H), 7.19 (dd, J=2.4, 8.0 Hz, 1H), 7.02 (d, J=8.0 Hz, 1H), 2.50 (s, 3H), 1.92-1.79 (m, 1H), 1.02-0.94 (m, 2H), 0.91-0.81 (m, 2H).

Step 2

To a solution of 5-cyclopropyl-2-methylpyridine (300 mg, 2.25 mmol) in THF (10 mL) was added n-BuLi (1.80 mL, 2 M in hexane, 4.50 mmol) at −70° C. After 30 minutes, tert-butyl 4-oxopiperidine-1-carboxylate (537 mg, 2.70 mmol) was added at −70° C., and the mixture was stirred at −70° C. for 2 hours. To the mixture was added a saturated aqueous solution of NH₄Cl (20 mL). The mixture was extracted with EtOAc (2×20 mL). The combined organic layers were dried over Na₂SO₄, filtered, concentrated, and purified by silica gel chromatography (PE/EtOAc=5/1 to 3/1) to give tert-butyl 4-((5-cyclopropylpyridin-2-yl)methyl)-4-hydroxypiperidine-1-carboxylate (280 mg, 37.4%). ¹H-NMR (400 MHz, CDCl₃) S_{H} 8.30 (d, J=2.0 Hz, 1H), 7.26-7.22 (m, 1H), 6.98 (d, J=8.0 Hz, 1H), 3.87-3.65 (m, 2H), 3.33-3.12 (m, 2H), 2.83 (s, 2H), 1.93-1.81 (m, 1H), 1.50-1.45 (m, 4H), 1.45 (s, 10H), 1.07-0.94 (m, 2H), 0.74-0.63 (m, 2H).

Step 3

To a solution of tert-butyl 4-((5-cyclopropylpyridin-2-yl)methyl)-4-hydroxypiperidine-1-carboxylate (300 mg, 0.90 mmol) was added HCl/Dioxane (10 mL, 4 M, 40.0 mmol) at 25° C. The mixture was stirred for 16 hours under nitrogen. The mixture was concentrated to give 4-((5-cyclopropylpyridin-2-yl)methyl)piperidin-4-ol hydrochloride (250 mg, crude), which was used directly in the next reaction.

Step 4

A solution of 2-(pyrimidin-4-yl)nicotinic acid (89.8 mg, 3.78 mmol), HOBt (100 mg, 0.74 mmol), EDCI (142 mg, 0.74 mmol), DIPEA (0.32 mL, 1.85 mmol) and 4-((5-cyclopropylpyridin-2-yl)methyl)piperidin-4-ol hydrochloride (100 mg, 0.37 mmol) in DMF (2 mL) was stirred at 0° C. for 2 hours. The mixture was filtered, concentrated, and purified by prep-HPLC (Column: Phenomenex Gemini-NX 80*40 mm*3 um; Condition: A=water (0.05% NH₃H₂O+10 mM NH₄HCO₃)-B=ACN; Begin B: 15; End B: 45; Gradient Time (min): 8; 100% B Hold Time (min): 2.5) to afford (4-((5-cyclopropylpyridin-2-yl)methyl)-4-hydroxypiperidin-1-yl)(2-(pyrimidin-4-yl)pyridin-3-yl)methanone (150 mg, impure). ¹H-NMR (400 MHz, CDCl₃) δ_{H} 9.29-9.17 (m, 1H), 8.92-8.82 (m, 1H), 8.77-8.69 (m, 1H), 8.31-8.27 (m, 1H), 8.26-8.21 (m, 1H), 7.74-7.65 (m, 1H), 7.48-7.40 (m, 1H), 7.26-7.24 (m, 1H), 7.04-6.95 (m, 1H), 4.57-4.40 (m, 1H), 3.52-3.10 (m, 3H), 2.91-2.79 (m, 2H), 1.93-1.81 (m, 1H), 1.69 (d, J=12.0 Hz, 1H), 1.50-1.26 (m, 4H), 1.07-0.96 (m, 2H), 0.74-0.65 (m, 2H). LCMS purity 95%, MS ESI calcd. for C₂₄H₂₆N₅O₂ [M+H]⁺ 416, found 416.

Step 5

To a mixture of (4-((5-cyclopropylpyridin-2-yl)methyl)-4-hydroxypiperidin-1-yl)(2-(pyrimidin-4-yl)pyridin-3-yl)methanone (140 mg, 0.34 mmol) in DCM (5 mL) was added DAST (108 mg, 0.67 mmol) at 0° C. The mixture was stirred at 0° C. for 15 minutes. The mixture was slowly powered into ice-water (20 mL), the aqueous layer was extracted with DCM (2×20 mL). The combined organic layers were washed with NaHCO₃ (20 mL) and brine (10 mL), dried over anhydrous Na₂SO₄ and concentrated. The residue was purified by SFC (Column: DAICEL CHIRALCEL OD-H (250 mm*30 mm, 5 um); Condition: A=0.1% NH₃H₂O B=EtOH; Begin B: 35%; End 35%) to give (4-((5-cyclopropylpyridin-2-yl)methyl)-4-fluoropiperidin-1-yl)(2-(pyrimidin-4-yl)pyridin-3-yl)methanone (7.3 mg 5.21%). ¹H-NMR (400 MHz, CDCl₃) δ_{H} 9.18 (s, 0.3H), 8.85 (d, J=5.2 Hz, 1H), 8.76-8.65 (m, 1.7H), 8.39-8.30 (m, 1H), 8.28-8.14 (m, 1H), 7.75-7.58 (m, 1H), 7.49-7.38 (m, 1H), 7.11 (d, J=7.6 Hz, 2H), 4.74-4.41 (m, 1H), 3.49-3.33 (m, 1H), 3.28-2.99 (m, 4H), 2.09-1.80 (m, 4H), 1.69-1.63 (m, 1H), 1.11-0.95 (m, 2H), 0.82-0.64 (m, 2H). LCMS purity 99%, MS ESI calcd. for C₂₄H₂₅FN₅O [M+H]⁺ 418, found 418.

### Example 10. Synthesis of (4-fluoro-4-phenylpiperidin-1-yl)(2-(pyrimidin-4-yl)pyridin-3-yl)methanone (Cmpd 8)

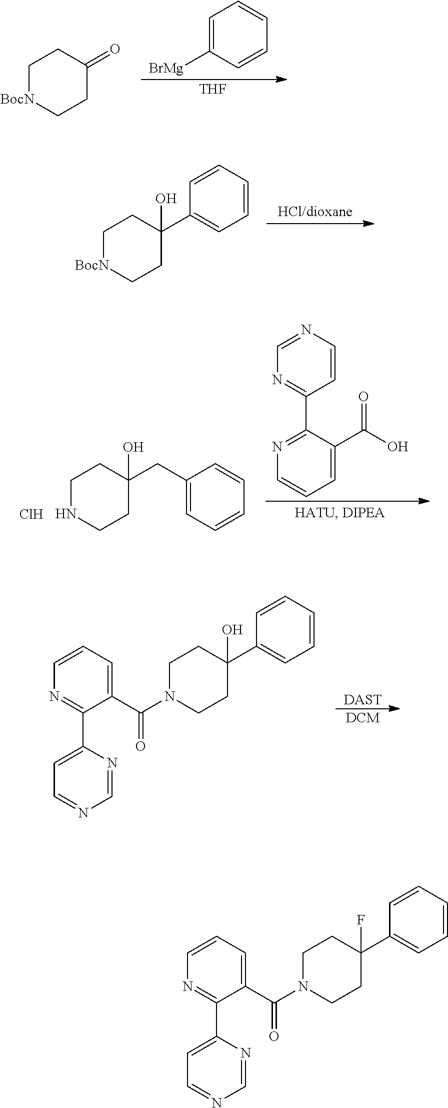

Step 1

To a solution of tert-butyl 4-oxopiperidine-1-carboxylate (3.0 g, 15.0 mmol) in THE (20 mL) was added phenyl magnesium bromide (15.0 mL, 45.0 mmol) dropwise at 0° C. under nitrogen. The mixture was stirred at 0° C. for 30 minutes and then warmed to 20° C. and stirred for 1 hour. The mixture was slowly poured into ice-water (100 mL) and stirred for 20 minutes. The mixture was extracted with EtOAc (3×400 mL). The combined organic layers were washed with brine (2×200 mL), dried over anhydrous Na₂SO₄, filtered and concentrated. The residue was purified by silica gel chromatography (10˜30% of EtOAc in PE) to give tert-butyl 4-hydroxy-4-phenylpiperidine-1-carboxylate (2.6 g, impure). ¹H-NMR (400 MHz, CDCl₃) δ_{H} 7.42-7.50 (m, 2H), 7.32-7.39 (m, 2H), 7.28-7.26 (m, 1H), 2.79-3.58 (m, 4H), 1.61-2.02 (m, 4H), 1.47-1.49 (s, 9H).

Step 2

To a solution of tert-butyl 4-hydroxy-4-phenylpiperidine-1-carboxylate (1.8 g, 6.48 mmol) in dioxane (20 mL) was added HCl/dioxane (4 M, 20 mL). The mixture was stirred at 25° C. for 16 h, the reaction mixture was concentrated under reduced pressure to give 4-phenylpiperidin-4-ol hydrochloride (1.4 g, crude), which was used directly in the next reaction.

Step 3

To a solution of 2-(pyrimidin-4-yl)nicotinic acid (657 mg, 3.27 mmol) in DMF (5 mL) was added HATU (1.86 g, 4.90 mmol) and DIPEA (1.70 mL, 9.80 mmol) at 25° C. The mixture was stirred for 30 min at 25° C. 4-Phenylpiperidin-4-ol hydrochloride (700 mg, 3.27 mmol) was added, and the mixture was stirred for 2 hours at 25° C. The mixture was concentrated, and the residue was purified by prep-HPLC (Column Phenomenex Gemini NX C18 150*40 mm*5 μm Condition A=water (10 mM NH4HCO3)-B=ACN Begin B 10 End B 40 Gradient Time (min) 10 100% B Hold Time (min) 2 Flow Rate (ml/min) 60 Injections 9) to afford (4-hydroxy-4-phenylpiperidin-1-yl)(2-(pyrimidin-4-yl)pyridin-3-yl)methanone (300 mg, 37.6%). ¹H-NMR (400 MHz, CDCl₃) δ_{H} 9.13-9.34 (m, 1H), 8.70-8.92 (m, 2H), 8.28 (br dd, J=19.6, 4.8 Hz, 1H), 7.70-7.84 (m, 1H), 7.49 (br dd, J=7.7, 4.6 Hz, 3H), 7.37-7.45 (m, 2H), 7.31-7.37 (m, 1H), 4.64-4.84 (m, 1H), 3.28-3.70 (m, 3H), 1.92-2.40 (m, 3H), 1.60 (br d, J=14.8 Hz, 1H); LC-ELSD/MS purity 99%, MS ESI calcd. for C₂₁H₂₁N₄O₂ [M+H]⁺ 361.2, found 361.2.

Step 4

To a mixture of (4-hydroxy-4-phenylpiperidin-1-yl)(2-(pyrimidin-4-yl)pyridin-3-yl)methanone (100 mg, 0.2774 mmol) in DCM (3 mL) was added DAST (89.4 mg, 0.559 mmol) at 0° C. The mixture was stirred at 0° C. for 30 minutes. The residue was poured into a mixture of ice-water and NaHCO₃ (80 mL) and stirred for 20 minutes. The aqueous phase was extracted with DCM (3×30 mL). The combined organic layers were washed with brine (2×20 mL), dried over anhydrous Na₂SO₄, filtered and concentrated. The residue was purified by HPLC (Column Boston Prime C18 150*30 mm*5 um Condition A=water (0.05%₀NH₃·H₂O+10 mM NH₄HCO₃)-B=ACN Begin B 40 End B 70 Gradient Time (min) 7 100% B Hold Time (min) 0 Flow Rate (ml/min) 25 Injections 5)) to afford (4-fluoro-4-phenylpiperidin-1-yl)(2-(pyrimidin-4-yl)pyridin-3-yl)methanone (19.7 mg, 19%). ¹H-NMR (400 MHz, CDCl₃) δ_{H} 9.20-9.30 (m, 1H), 8.74-8.93 (m, 2H), 8.22-8.34 (m, 1H), 7.69-7.81 (m, 1H), 7.48 (dd, J=7.6, 5.2 Hz, 1H), 7.32-7.43 (m, 5H), 4.73-4.94 (m, 1H), 3.59 (br s, 1H), 3.16-3.44 (m, 2H), 2.09-2.42 (m, 2H), 1.76-1.99 (m, 1H), 1.21-1.38 (m, 1H). ¹⁹F-NMR (376.5 MHz, CDCl₃) δ_{F} −161.675. LC-ELSD/MS purity 99%, MS ESI calcd. for C₂₁H₂₀FN₄O [M+H]⁺ 363.1, found 363.1.

### Example 11. Synthesis of 3-fluoro-4-((4-fluoro-1-(2-(pyrimidin-4-yl)nicotinoyl)piperidin-4-yl)methyl)benzonitrile (Cmpd 9)

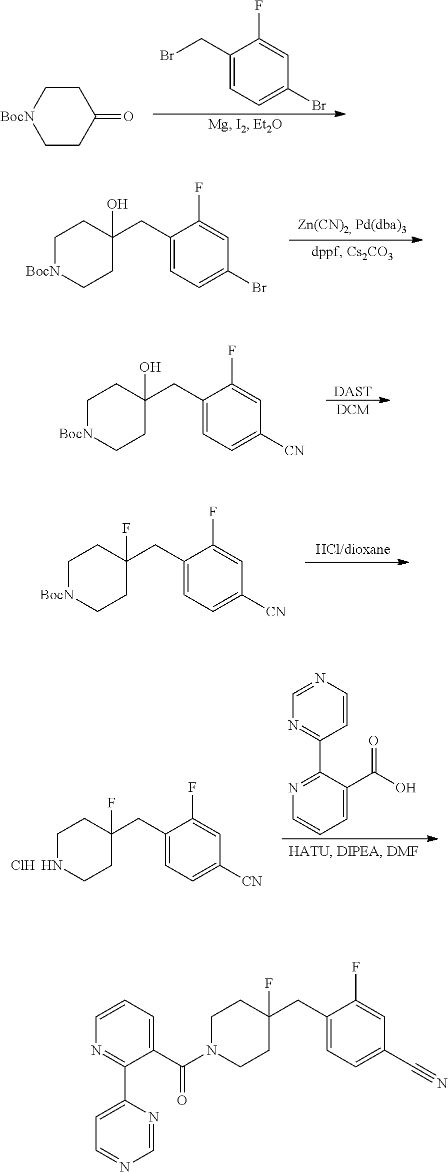

Step 1

To a suspension of Mg (3.90 g, 150 mmol) and I₂ (100 mg, 0.3906 mmol) in Et₂O (60 mL) was added 4-bromo-1-(bromomethyl)-2-fluorobenzene (9.37 g, 37.5 mmol) in Et₂O (10 mL) at 20° C. under nitrogen. The reaction mixture was heated to 40° C. and stirred for 30 minutes. Then tert-butyl 4-oxopiperidine-1-carboxylate (597 mg, 3 mmol) in diethyl ether (10 mL) was slowly added. The mixture was heated at 40° C. for 1 hour. The mixture was cooled and a saturated aqueous solution of NH₄Cl (30 mL) was added. The mixture was extracted with EtOAc (20 mL×2), and the combined organic layers were washed with brine (50 mL), dried over Na₂SO₄, filtered and concentrated. The crude product was purified by silica gel chromatography (0-10% of EtOAc in PE) to afford tert-butyl 4-(4-bromo-2-fluorobenzyl)-4-hydroxypiperidine-1-carboxylate (1.1 g, 94.8%). ¹H-NMR (400 MHz, CDCl₃)^{6H}=7.25-7.22 (m, 2H), 7.15-7.06 (m, 1H), 3.85 (s, 2H), 3.19-3.02 (m, 2H), 2.85-2.74 (m, 2H), 1.68-1.57 (m, 2H), 1.50-1.41 (m, 12H).

Step 2

To a solution of tert-butyl 4-(4-bromo-2-fluorobenzyl)-4-hydroxypiperidine-1-carboxylate (1.1 g, 2.83 mmol) in DMF (40 mL) was added Zn(CN)₂ (2.29 g, 19.8 mmol) at 20° C. under nitrogen. Then Pd₂(dba)₃ (440 mg, 0.4811 mmol) and dppf (737 mg, 1.33 mmol) were added. The mixture was stirred at 130° C. for 16 hours. The mixture was cooled and ice-water (70 mL) was added. The mixture was extracted with EtOAc (70 mL×2). The combined organic layers were dried over Na₂SO₄, concentrated and purified by silica gel chromatography (0˜90% of EtOAc in PE) to give tert-butyl 4-(4-cyano-2-fluorobenzyl)-4-hydroxypiperidine-1-carboxylate (400 mg, 42.2%). ¹H-NMR (400 MHz, CDCl₃) δ_{H}=7.45-7.34 (m, 3H), 3.95-3.77 (m, 2H), 3.18-3.00 (m, 2H), 2.87 (s, 2H), 1.69-1.58 (m, 2H), 1.45 (s, 12H).

Step 3

To a solution of tert-butyl 4-(4-bromo-2-fluorobenzyl)-4-hydroxypiperidine-1-carboxylate (400 mg, 1.19 mmol) in DCM (20 mL) was added DAST (397 mg, 2.38 mmol) at 0° C. The mixture was stirred at 0° C. for 5 minutes. The reaction mixture was slowly poured into ice-water (30 mL) and the mixture was extracted with DCM (2×30 mL). The combined organic layers were washed with an aqueous solution of NaHCO₃ (20 mL), then brine (10 mL), dried over anhydrous Na₂SO₄, filtered and concentrated to give tert-butyl 4-(4-cyano-2-fluorobenzyl)-4-fluoropiperidine-1-carboxylate (350 mg, 87.5%), which was used directly in the next reaction.

Step 4

To a solution of tert-butyl 4-(4-cyano-2-fluorobenzyl)-4-fluoropiperidine-1-carboxylate (350 mg, 1.04 mmol) was added HCl/dioxane (10 mL, 4M, 40.0 mmol) at 15° C. The mixture was stirred at 15° C. for 16 hours under nitrogen. The mixture was concentrated to give 3-fluoro-4-((4-fluoropiperidin-4-yl)methyl)benzonitrile hydrochloride (250 mg, crude), which was used directly in the next step.

Step 5

A mixture of 2-(pyrimidin-4-yl)nicotinic acid (201 mg, 1.00 mmol), HATU (695 mg, 1.83 mmol), DIPEA (0.796 mL, 4.58 mmol) and 3-fluoro-4-((4-fluoropiperidin-4-yl)methyl)benzonitrile hydrochloride (250 mg, 0.917 mmol) in DMF (2 mL) was stirred at 15° C. for 16 hours. The mixture was poured into water (20 mL) and stirred for 20 minutes. The mixture was extracted with EtOAc (30 mL). The organic layer was washed with brine (2×20 mL), dried over anhydrous Na₂SO₄, filtered and concentrated. The residue was purified by SFC (Column: DAICEL CHIRALPAK AS (250 mm*30 mm, 10 um), Condition 0.1% NH₃H₂O EtOH, Begin B 25, End B 25, Flow Rate (ml/min) 60) to afford the desired product (100 mg). The residue was purified by prep-TLC (DCM/MeOH=10/1, eluted twice) to afford 3-fluoro-4-((4-fluoro-1-(2-(pyrimidin-4-yl)nicotinoyl)piperidin-4-yl)methyl)benzonitrile (41.6 mg, 10%). ¹H-NMR (400 MHz, CDCl₃) δ_{H}=9.18 (s, 1H), 9.01 (s, 1H), 8.94-8.83 (m, 1H), 8.80-8.70 (m, 1H), 8.26 (s, 1H), 7.79-7.60 (m, 1H), 7.50-7.33 (m, 4H), 4.78-4.50 (m, 1H), 3.58-3.31 (m, 1H), 3.28-2.97 (m, 4H), 2.14-1.82 (m, 2H), 1.67 (s, 1H). LCMS purity 100%, MS ESI calcd. for C₂₃H₂₀F₂N₅O [M+H]⁺ 420, found 420.

### Example 12. Synthesis of (4-fluoro-4-((5-fluoropyridin-2-yl)methyl)piperidin-1-yl)(2-(pyrimidin-4-yl)pyridin-3-yl)methanone (Cmpd 10)

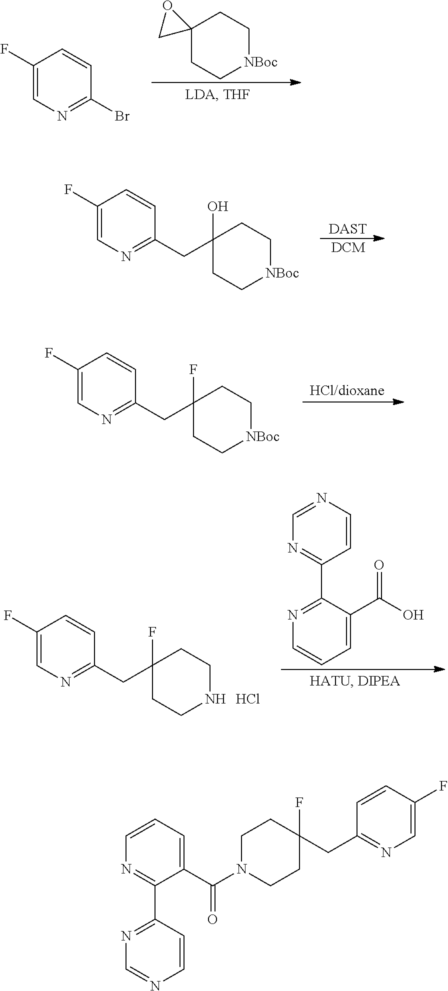

Step 1

To a solution of 2-bromo-5-fluoropyridine (100 mg, 0.568 mmol) in THF (5 mL) was added slowly to a solution of n-BuLi (0.22 mL, 2.5 M in hexane, 0.5682 mmol) at −78° C., and the mixture was stirred at −78° C. for 30 minutes. BF₃·Et₂O (0.72 ml 5.68 mmol) and CuI (1.08 g, 5.68 mmol) was added into the reaction and stirred at −78° C. for 10 minutes. To the resulting mixture was added a solution of tert-butyl 1-oxa-6-azaspiro[2.5]octane-6-carboxylate (1.2 g, 5.62 mmol) in THE (5 mL) dropwise at −70° C. under nitrogen. After stirring at −70° C. for 1 hour, the mixture was poured into water (20 mL) and stirred for 20 min. The mixture was extracted with EtOAc (3×20 mL). The combined organic layers were washed with brine (2×100 mL), dried over anhydrous Na₂SO₄, filtered and concentrated. The residue was purified by flash chromatography (0˜30% of EtOAc in PE) to give tert-butyl 4-((5-fluoropyridin-2-yl)methyl)-4-hydroxypiperidine-1-carboxylate (230 mg, impure). ¹H-NMR (400 MHz, CDCl₃) δ 8.39 (m, 1H), 7.44-7.35 (m, 1H), 7.13 (m, 1H), 3.80 (br s, 2H), 3.23 (s, 2H), 1.58 (m, 3H), 1.52-1.48 (m, 2H), 1.46 (s, 9H).

Step 2

To a mixture of tert-butyl 4-((5-fluoropyridin-2-yl)methyl)-4-hydroxypiperidine-1-carboxylate (230 mg, 0.741 mmol) in DCM (5 mL) was added DAST (0.195 mL, 1.48 mmol) at 0° C. The mixture was stirred at 20° C. for 30 minutes. The mixture was poured into water (50 mL) and stirred for 20 min. The mixture was extracted with DCM (3×20 mL). The combined organic layers were washed with brine (2×50 mL), dried over anhydrous Na₂SO₄, filtered and concentrated to give tert-butyl 4-fluoro-4-((5-fluoropyridin-2-yl)methyl)piperidine-1-carboxylate (200 mg, crude), which was used directly in the next step. ¹H-NMR (400 MHz, CDCl₃) δ 8.46-8.33 (m, 1H), 7.45-7.31 (m, 1H), 7.26-7.15 (m, 1H), 3.90 (s, 2H), 3.50 (m, 2H), 3.13-2.99 (m, 2H), 2.14-1.98 (m, 1H), 1.79-1.57 (m, 3H), 1.46 (m, 9H).

Step 3

To a mixture of tert-butyl 4-fluoro-4-((5-fluoropyridin-2-yl)methyl)piperidine-1-carboxylate (0.2 g, 0.6402 mmol) in dioxane (5 mL) was added HCl/dioxane (5 mL, 4M in dioxane, 18.1 mmol). The mixture was stirred at 25° C. for 4 hours. The mixture was concentrated to give 5-fluoro-2-((4-fluoropiperidin-4-yl)methyl)pyridine hydrochloride (150 mg, impure). ¹H-NMR (400 MHz, CDCl₃) δ 8.67-8.55 (m, 1H), 8.15-7.99 (m, 1H), 7.85-7.65 (m, 1H), 4.08-3.91 (m, 2H), 3.13-2.91 (m, 2H), 2.10 (s, 4H), 1.51-1.46 (m, 2H).

Step 4

To a solution of 2-(pyrimidin-4-yl)nicotinic acid (161 mg, 0.8041 mmol), 5-fluoro-2-((4-fluoropiperidin-4-yl)methyl)pyridine hydrochloride (200 mg, 0.8041 mmol) and HATU (456 mg, 1.20 mmol) in DMF (5 mL) was added DIPEA (0.42 mL, 2.41 mmol). The mixture was stirred at 20° C. for 12 hours. The mixture was poured into H₂O (50 mL) and stirred for 20 minutes. The mixture was extracted with EtOAc (3×20 mL). The combined organic layers were washed with brine (2×20 mL), dried over anhydrous Na₂SO₄, filtered and concentrated. The residue was purified by SFC (Column DAICEL CHIRALCEL OD-H (250 mm*30 mm, 5 um) Condition A=0.1% NH₃H₂O B=EtOH Begin B 20% End B 20% Gradient Time(min) 100% B Hold Time(min) Flow Rate (ml/min): 60 Injections 90) to afford (4-fluoro-4-((5-fluoropyridin-2-yl)methyl)piperidin-1-yl)(2-(pyrimidin-4-yl)pyridin-3-yl)methanone (20 mg, 6.3%). ¹H-NMR (400 MHz, CDCl₃) δ 9.20 (s, 1H), 8.90 (d, J=13.1 Hz, 1H), 8.75 (s, 1H), 8.40 (s, 1H), 8.24 (s, 1H), 7.68 (m, 1H), 7.51-7.34 (m, 2H), 7.31-7.27 (m, 1H), 4.75-4.48 (m, 1H), 3.43 (s, 1H), 3.27-3.08 (m, 4H), 2.08-1.90 (m, 2H), 1.73-1.57 (m, 2H). ¹⁹F NMR (400 MHz, CDCl₃) δ_{F} −129.492, −160.008. LC-ELSD/MS purity ≥98%, MS ESI calcd. for C₂₁H₁₉F₂N₅O [M+H]⁺ 396.2, found 396.2.

### Example 13. Synthesis of 3,5-difluoro-4-((4-fluoro-1-(2-(pyrimidin-4-yl)nicotinoyl)piperidin-4-yl)methyl)benzonitrile (Cmpd 11)

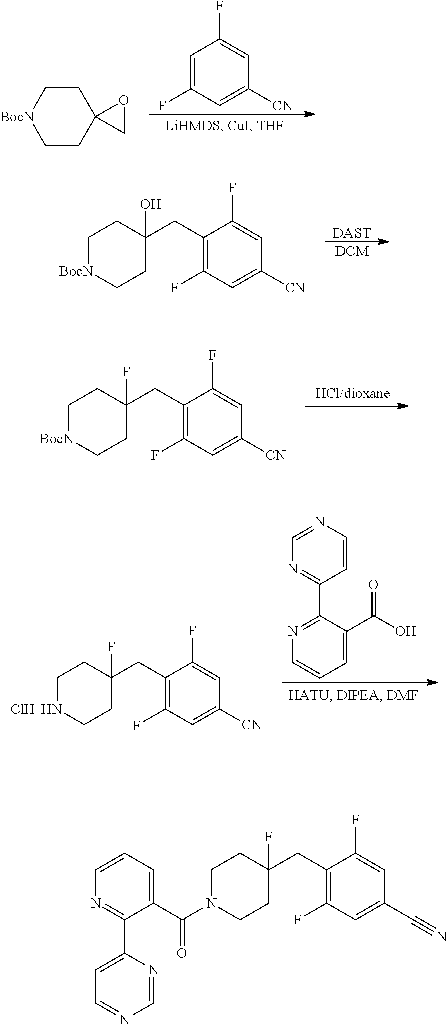

Step 1

To a solution of 3,5-difluorobenzonitrile (2 g, 14.3 mmol) in THE (20 mL) was added a solution of LiHMDS (28.6 mL, 1M in hexane, 28.6 mmol) at −78° C. The mixture was stirred at −78° C. for 30 minutes, and BF₃·EtO (2.02 g, 14.3 mmol) was added. A solution of tert-butyl 1-oxa-6-azaspiro[2.5]octane-6-carboxylate (1.68 g, 7.89 mmol) in THE (10 mL) was added dropwise to the mixture and stirring was continued for 1 hour. The reaction mixture was poured slowly into H₂O (20 mL). The aqueous phase was extracted with EtOAc (3×100 mL). The combined organic layers were washed with brine (2×20 mL), dried over anhydrous Na₂SO₄ and concentrated. The residue was purified by flash chromatography (5˜30% of EtOAc in PE) and further purified by prep-HPLC (Column Phenomenex Gemini-NX 80*30 mm*3 um, Condition A=water (10 mM NH₄HCO₃)-B=ACN, Begin B 36, End B 66, Gradient Time(min): 9, 100% B Hold Time(min): 2.5) to give tert-butyl 4-(4-cyano-2,6-difluorobenzyl)-4-hydroxypiperidine-1-carboxylate (100 mg, 3.95%). ¹H-NMR (400 MHz, CDCl₃) δ_{H}=7.24-7.21 (m, 2H), 3.88 (s, 2H), 3.09 (s, 2H), 2.90 (s, 2H), 1.45 (s, 10H).

Step 2

To a mixture of tert-butyl 4-(4-cyano-2,6-difluorobenzyl)-4-hydroxypiperidine-1-carboxylate (100 mg, 0.284 mmol) in DCM (10 mL) was added DAST (94.7 mg, 0.5674 mmol) at 0° C. The mixture was stirred at 0° C. for 5 minutes. The mixture was slowly poured into ice-water (30 mL) and extracted with DCM (2×30 mL). The combined organic layers were washed with NaHCO₃ (20 mL), then brine (10 mL), dried over anhydrous Na₂SO₄, concentrated to give tert-butyl 4-(4-cyano-2,6-difluorobenzyl)-4-fluoropiperidine-1-carboxylate (90 mg, 89.9%) which was used directly for the next reaction.

Step 3

To a solution of tert-butyl 4-(4-cyano-2,6-difluorobenzyl)-4-fluoropiperidine-1-carboxylate (90 mg, 0.254 mmol) was added HCl/MeOH (5 mL, 4M, 40.0 mmol) at 15° C. The mixture was stirred at 15° C. for 16 hours under nitrogen. The mixture was concentrated to give 3,5-difluoro-4-((4-fluoropiperidin-4-yl)methyl)benzonitrile hydrochloride (80 mg, crude), which was used directly in the next step.

Step 4

To a solution of 2-(pyrimidin-4-yl)nicotinic acid (66.4 mg, 0.33 mmol), HATU (209 mg, 0.55 mmol), DIPEA (0.142 mL, 0.825 mmol) and 3,5-difluoro-4-((4-fluoropiperidin-4-yl)methyl)benzonitrile hydrochloride (80 mg, 0.275 mmol) in DMF (2 mL) was stirred at 15° C. for 16 hours. The mixture was poured into water (10 mL) and stirred for 20 minutes. The mixture was extracted with EtOAc (10×2 mL). The combined organic layers were washed with brine (2×10 mL), dried over anhydrous Na₂SO₄, filtered and concentrated. The residue was purified by silica gel chromatography [two elutions: 1^{st} elution: (PE/EtOAc=1/0 to 0/1) 2^{nd} elution: (DCM/MEOH=0/1 to 10/1)] to afford 30 mg, which was further was purified by SFC (Column DAICEL CHIRALPAK AS (250 mm*30 mm, 10 um), Condition A=0.1% NH₃H₂O B=EtOH, Begin B 20, End B 20, Flow Rate (ml/min) 55) to afford 3,5-difluoro-4-((4-fluoro-1-(2-(pyrimidin-4-yl)nicotinoyl)piperidin-4-yl)methyl)benzonitrile (3.1 mg, 10%). ¹H-NMR (400 MHz, CDCl₃) δ_{H}=9.18 (s, 0.4H), 8.99 (s, 0.6H), 8.94-8.85 (m, 1H), 8.80-8.72 (m, 1H), 8.27 (s, 1H), 7.75-7.64 (m, 1H), 7.47 (br s, 1H), 7.26 (s, 2H), 4.83-4.51 (m, 1H), 3.54-3.33 (m, 1H), 3.10 (d, J=19.6 Hz, 3H), 2.06-1.94 (m, 2H), 0.91-0.78 (m, 3H). LCMS purity 100%, MS ESI calcd. for C₂₃H₁₉F₃N₅O [M+H]⁺ 438, found 438. ¹⁹F NMR (400 MHz, CDCl₃) δ_{F} −108.0, −161.9

### Example 14. Synthesis of (4-fluoro-1-(2-(pyrimidin-4-yl)nicotinoyl)piperidin-4-yl)(4-(trifluoromethyl)phenyl)methanone (Cmpd 12)

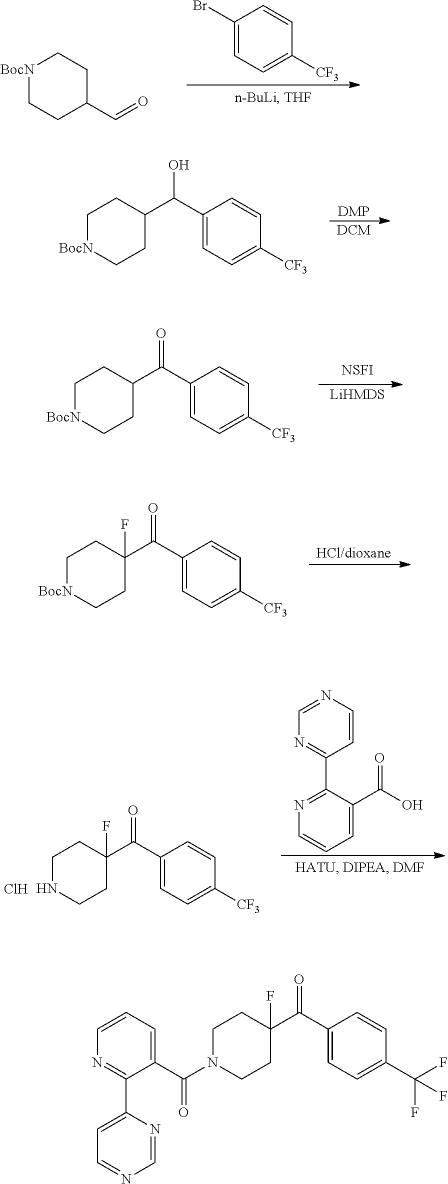

Step 1

To a solution of 1-bromo-4-(trifluoromethyl)benzene (5.0 g, 22.2 mmol) in THE (50 mL) at −78° C. was added slowly a solution of n-BuLi (13.3 mL, 2.5 M in hexane, 33.3 mmol), and the mixture was stirred at −78° C. for 30 minutes. To this mixture at −70° C. was added dropwise a solution of tert-butyl 4-formylpiperidine-1-carboxylate (3.37 g in THF, 22.2 mmol) in THE (10 mL). After stirring at −70° C. for 1 hour, the mixture was poured into a NH₄Cl solution (200 mL) and stirred for 20 minutes. The mixture was extracted with EtOAc (3×100 mL). The combined organic layers were washed with brine (2×200 mL), dried over anhydrous Na₂SO₄, filtered and concentrated. The residue was purified by silica gel chromatography (10˜30% of EtOAc in PE) to give tert-butyl 4-(hydroxy(4-(trifluoromethyl)phenyl)methyl)piperidine-1-carboxylate (3.0 g, impure). ¹H-NMR (400 MHz, CDCl₃) δ_{H} 7.62 (d, J=8.0 Hz, 2H), 7.44 (d, J=8.0 Hz, 2H), 4.49 (d, J=5.8 Hz, 1H), 4.13 (m, 2H), 2.64 (d, J=10.3 Hz, 2H), 1.90 (d, J=12.8 Hz, 1H), 1.80-1.69 (m, 2H), 1.45 (s, 9H), 1.36-1.18 (m, 5H).

Step 2

To a solution of tert-butyl 4-(hydroxy(4-(trifluoromethyl)phenyl)methyl)piperidine-1-carboxylate (3.0 g, 8.34 mmol) in DCM (30 mL) at 25° C. was added DMP (7.04 g, 16.6 mmol) in portions. The reaction was stirred at 25° C. for 0.5 hour. The reaction mixture was poured into a mixture of saturated aqueous NaHCO₃/Na₂S₂O₃ (1:1) (500 mL). The mixture was extracted with DCM (2×100 mL). The combined organic layers were washed with saturated aqueous NaHCO₃/Na₂S₂O₃ (1:1) (2×200 mL), dried over anhydrous Na₂SO₄, filtered and concentrated. The residue was purified by silica gel chromatography (10˜30% of EtOAc in PE) to give tert-butyl 4-(4-(trifluoromethyl)benzoyl)piperidine-1-carboxylate (2.7 g, 91%). ¹H-NMR (400 MHz, CDCl₃) δ_{H} 8.04 (d, J=8.3 Hz, 2H), 7.75 (d, J=8.1 Hz, 1H), 7.83-7.68 (m, 1H), 4.16 (s, 2H), 3.40 (m, 1H), 2.92 (m, 2H), 2.53-2.37 (m, 1H), 1.92-1.61 (m, 5H), 1.47 (s, 9H), 1.46 (s, 2H), 0.91 (m, 1H).

Step 3

To a solution tert-butyl 4-(4-(trifluoromethyl)benzoyl)piperidine-1-carboxylate (2.0 g, 5.59 mmol) in THE (20 mL) was added LiHMDS (8.38 mL, 8.38 mmol) dropwise at −78° C. under nitrogen. The mixture was stirred at −78° C. for 30 minutes. To this mixture was added a solution of NFSI (1.76 g, 5.59 mmol) in THF (2 mL) drop-wise at −78° C. The mixture was poured into a NaHCO₃ solution (200 mL) and stirred for 20 minutes. The mixture was extracted with EtOAc (3×100 mL). The combined organic layers were washed with brine (2×100 mL), dried over anhydrous Na₂SO₄, filtered and concentrated. The residue was purified by silica gel chromatography (5˜15% of EtOAc in PE) to afford tert-butyl 4-fluoro-4-(4-(trifluoromethyl)benzoyl)piperidine-1-carboxylate (1.2 g, 57.4%). ¹H-NMR (400 MHz, CDCl₃) δ_{H} 8.16 (d, J=8.4 Hz, 2H), 7.73 (d, J=8.3 Hz, 2H), 4.10 (s, 3H), 3.21 (m, 2H), 3.08-2.95 (m, 1H), 2.82 (m, 1H), 2.31-1.91 (m, 5H), 1.52-1.42 (m, 12H).

Step 4

To a solution of tert-butyl 4-fluoro-4-(4-(trifluoromethyl)benzoyl)piperidine-1-carboxylate (0.2 g, 0.53 mmol) in dioxane (5 mL) was added HCl/dioxane (5 mL, 4M in dioxane, 18.1 mmol) and the mixture was stirred at 25° C. for 4 hours. The mixture was concentrated to give (4-fluoropiperidin-4-yl)(4-(trifluoromethyl)phenyl)methanone hydrochloride (150 mg, crude). ¹H-NMR (400 MHz, CDCl₃) δ_{H} 8.16 (d, J=7.8 Hz, 2H), 7.84-7.67 (m, 2H), 4.20-4.01 (m, 1H), 3.65-2.99 (m, 3H), 2.82-2.57 (m, 1H), 2.35 (s, 1H), 2.21-1.94 (m, 3H), 1.73 (s, 3H).

Step 5

To a solution of 2-(pyrimidin-4-yl)nicotinic acid (116 mg, 0.5774 mmol), (4-fluoropiperidin-4-yl)(4-(trifluoromethyl)phenyl)methanone hydrochloride (180 mg, 0.5774 mmol) and HATU (329 mg, 0.8661 mmol) in DMF (5 mL) was added DIPEA (0.30 mL, 1.73 mmol). The mixture was stirred at 20° C. for 12 hours. The reaction mixture was poured into H₂O (50 mL) and stirred for 20 minutes. The mixture was extracted with EtOAc (3×20 mL). The combined organic layers were washed with brine (2×20 mL), dried over anhydrous Na₂SO₄, filtered and concentrated. The residue was purified by prep-HPLC (Phenomenex Gemini-NX C18 75*30 mm*3 um Condition A=water (0.225% FA)-B=ACN Begin B 42 End B 62 Gradient Time(min) 7 100% B Hold Time(min) 3 Flow Rate (ml/min) 30 Injections 5) to afford (4-fluoro-1-(2-(pyrimidin-4-yl)nicotinoyl)piperidin-4-yl)(4-(trifluoromethyl)phenyl)methanone (49.3 mg, 18.6%). ¹H-NMR (400 MHz, CDCl₃) δ_{H} 9.45-9.21 (m, 1H), 8.92 (s, 1H), 8.78 (d, J=4.3 Hz, 1H), 8.35-8.23 (m, 1H), 8.18 (d, J=8.3 Hz, 2H), 7.75 (d, J=8.3 Hz, 3H), 7.50 (d, J=4.8 Hz, 1H), 4.92-4.57 (m, 1H), 3.73-3.27 (m, 3H), 2.80-2.48 (m, 1H), 2.38-1.82 (m, 3H)¹⁹F-NMR (400 MHz, CDCl₃) δ_{F} −63.311, −165.406. LC-ELSD/MS purity ≥95%, MS ESI calcd. for C₂₃H₁₈F₄N₄O₂ [M+H]⁺ 459.1, found 459.1.

### Example 15 and Example 16. Synthesis of (R)-(4-fluoro-4-(hydroxy(4-(trifluoromethyl)phenyl)methyl)piperidin-1-yl)(2-(pyrimidin-4-yl)pyridin-3-yl)methanone (Cmpd 13) and Synthesis of (S)-(4-fluoro-4-(hydroxy(4-(trifluoromethyl)phenyl)methyl)piperidin-1-yl)(2-(pyrimidin-4-yl)pyridin-3-yl)methanone (Cmp 14)

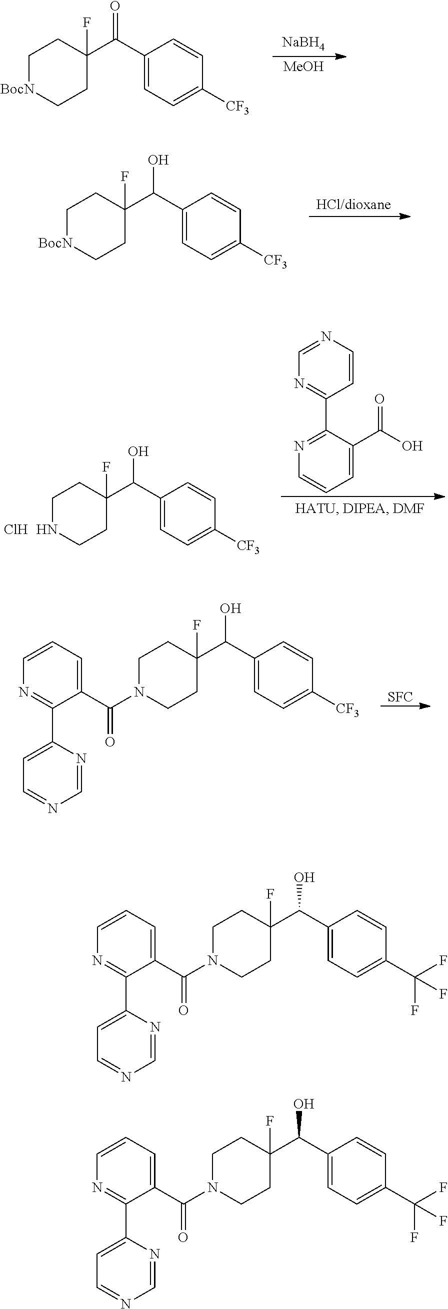

Step 1

To a solution of tert-butyl 4-fluoro-4-(4-(trifluoromethyl)benzoyl)piperidine-1-carboxylate (400 mg, 1.06 mmol) in MeOH (5 mL) was slowly added NaBH₄ (200 mg, 5.03 mmol) in portions at 25° C. The mixture was stirred at 25° C. for 1 hour. An aqueous Na₂S₂O₃ solution (20 mL) was added and stirring was continued for 30 minutes. The mixture was extracted with EtOAc (2×20 mL). The combined organic layers were washed with brine (2×20 mL), dried over anhydrous Na₂SO₄, filtered and concentrated to give racemic tert-butyl 4-fluoro-4-(hydroxy(4-(trifluoromethyl)phenyl)methyl)piperidine-1-carboxylate (400 mg, crude). ¹H-NMR (400 MHz, CDCl₃) δ_{H} 7.63 (d, J=8.3 Hz, 2H), 7.49 (d, J=8.1 Hz, 2H), 4.72 (d, J=13.4 Hz, 1H), 4.01 (s, 2H), 2.98 (s, 2H), 1.91-1.72 (m, 3H), 1.66-1.55 (m, 4H), 1.46 (s, 2H), 1.43 (s, 9H), 0.99 (m, 1H).

Step 2

To a solution of tert-butyl 4-fluoro-4-(hydroxy(4-(trifluoromethyl)phenyl)methyl)piperidine-1-carboxylate (0.4 g, 1.05 mmol) in dioxane (5 mL) was added HCl/dioxane (5 mL, 4M in dioxane, 18.1 mmol) and the mixture was stirred at 25° C. for 4 hours. The mixture was cooled and concentrated to give racemic-(4-fluoropiperidin-4-yl)(4-(trifluoromethyl)phenyl)methanol hydrochloride (360 mg, crude). ¹H-NMR (400 MHz, CDCl₃) δ_{H} 7.64 (d, J=8.5 Hz, 2H), 7.54-7.47 (m, 2H), 4.77-4.57 (m, 1H), 4.04 (s, 1H), 3.58 (s, 4H), 2.97 (s, 2H), 0.98 (d, J=8.0 Hz, 4H).

Step 3

To a solution of 2-(pyrimidin-4-yl)nicotinic acid (229 mg, 1.14 mmol), racemic-(4-fluoropiperidin-4-yl)(4-(trifluoromethyl)phenyl)methanol hydrochloride (360 mg, 1.14 mmol) and HATU (650 mg, 1.71 mmol) in DMF (5 mL) was added DIPEA (0.59 mL, 1.73 mmol). The mixture was stirred at 20° C. for 12 hours. The mixture was poured into H₂O (50 mL) and stirred for 20 minutes. The mixture was extracted with EtOAc (3×20 mL). The combined organic layers were washed with brine (2×20 mL), dried over anhydrous Na₂SO₄, filtered and concentrated. The residue was purified by SFC (Column DAICEL CHIRALPAK IG (250 mm*30 mm, 10 um) Condition A=0.1% NH₃H₂O B=EtOH Begin B 35% End B 35% Gradient Time(min) 100% B Hold Time(min) Flow Rate (ml/min) 70 Injections 55) to give:

(R)-(4-fluoro-4-(hydroxy(4-(trifluoromethyl)phenyl)methyl)piperidin-1-yl)(2-(pyrimidin-4-yl)pyridin-3-yl)methanone (58.5 mg, 32.5%). ¹H-NMR (400 MHz, CDCl₃) δ_{H} 9.19 (s, 1H), 8.91-8.62 (m, 3H), 8.33-8.13 (m, 1H), 7.73-7.57 (m, 3H), 7.57-7.37 (m, 3H), 4.94-4.59 (m, 2H), 3.58-2.95 (m, 3H), 2.83-2.59 (m, 1H), 2.18-1.83 (m, 2H), 1.57-1.41 (m, 1H)¹⁹F NMR (400 MHz, CDCl₃) δ_{F} −162.565. LC-ELSD/MS purity ≥98%, ee %=100%; MS ESI calcd. for C₂₃H₂₀F₄N₄O₂[M+H]⁺ 461.1, found 461.1.

(S)-(4-fluoro-4-(hydroxy(4-(trifluoromethyl)phenyl)methyl)piperidin-1-yl)(2-(pyrimidin-4-yl)pyridin-3-yl)methanone (65.8 mg, 36.7%): ¹H NMR (400 MHz, CDCl₃) δ_{H} 8.83-8.56 (m, 3H), 8.27-8.04 (m, 1H), 7.69-7.50 (m, 3H), 7.47-7.31 (m, 3H), 4.81-4.52 (m, 2H), 3.51-2.58 (m, 4H), 2.33-1.72 (m, 2H), 1.51-1.21 (m, 1H). ¹⁹F NMR (400 MHz, CDCl₃) δ_{F} −162.565. LC-ELSD/MS purity ≥98%, ee %=100%; MS ESI calcd. for C₂₃H₂₀F₄N₄O₂[M+H]⁺ 461.1, found 461.1.

### Example 17 and Example 18. Synthesis of (S)-3-fluoro-4-((4-fluoro-1-(2-(pyrimidin-4-yl)nicotinoyl)piperidin-4-yl)(hydroxy)methyl)benzonitrile (Cmpd 15) and Synthesis of (R)-3-fluoro-4-((4-fluoro-1-(2-(pyrimidin-4-yl)nicotinoyl)piperidin-4-yl)(hydroxy)methyl)benzonitrile (Cmpd 16)

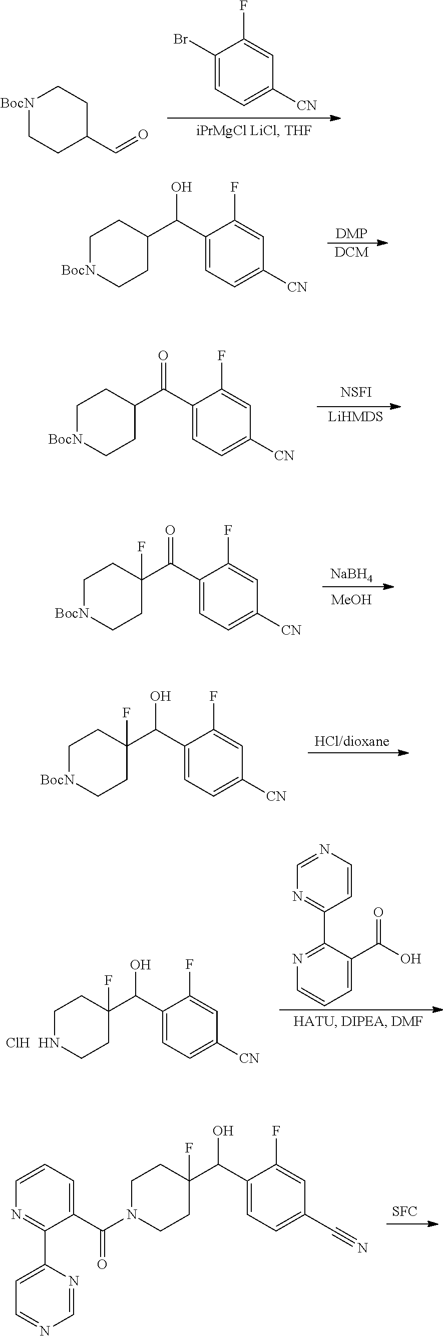

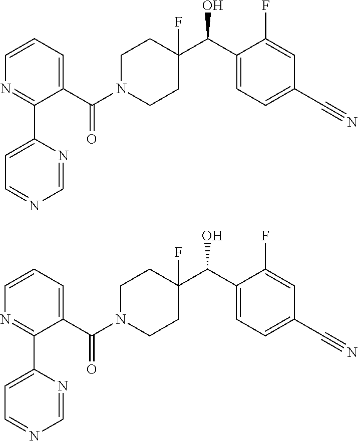 [SMILES: N#Cc1ccc([C@H](O)C2(F)CCN(C(=O)c3cccnc3-c3ccncn3)CC2)c(F)c1 ; N#Cc1ccc([C@@H](O)C2(F)CCN(C(=O)c3cccnc3-c3ccncn3)CC2)c(F)c1]

Step 1

To a solution of iPrMgCl LiCl (22.9 mL, 1.3 M, 29.8 mmol) in THE (10 mL) was added 4-bromo-3-fluorobenzonitrile (5 g, 24.9 mmol) in THE (10 mL). The mixture was stirred under nitrogen at −10° C. for 0.5 hours, and then tert-butyl 4-formylpiperidine-1-carboxylate (5 g, 23.4 mmol) in THF (50 mL) was added dropwise at −70° C. After stirring at −70° C. for 1 hour, the mixture was poured slowly into a saturated aqueous solution of NH₄Cl (40 mL). The mixture was extracted with EtOAc (3×20 mL). The combined organic layers were washed with brine (2×20 mL), dried over anhydrous Na₂SO₄, and concentrated. The residue was purified by silica gel chromatography (5˜30% of EtOAc in PE) to give tert-butyl 4-((4-cyano-2-fluorophenyl)(hydroxy)methyl)piperidine-1-carboxylate (6.6 g, 84.3%). ¹H-NMR (400 MHz, CDCl₃)^{6H}=7.67-7.56 (m, 1H), 7.54-7.43 (m, 1H), 7.36-7.31 (m, 1H), 4.92-4.82 (m, 1H), 4.26-3.93 (m, 2H), 2.71-2.52 (m, 2H), 2.11-2.01 (m, 1H), 1.85-1.66 (m, 2H), 1.44 (s, 9H), 1.40-1.23 (m, 3H).

Step 2

To a suspension of tert-butyl 4-((4-cyano-2-fluorophenyl)(hydroxy)methyl)piperidine-1-carboxylate (3 g, 8.97 mmol) in DCM (30 mL) was added DMP (7.58 g, 17.9 mmol). The reaction mixture was stirred at 60° C. for 0.5 hours. To the mixture was added an aqueous solution of NaHCO₃ (50 mL) and an aqueous solution of NaS₂O₃ (50 mL). The mixture was extracted with DCM (2×50 mL). The combined organic layers were washed with brine (50 mL), dried over Na₂SO₄, filtered and concentrated to give tert-butyl 4-(4-cyano-2-fluorobenzoyl)piperidine-1-carboxylate (2.5 g, 83.8%). ¹H-NMR (400 MHz, CDCl₃)^{6H}=7.90-7.78 (m, 1H), 7.60-7.52 (m, 1H), 7.50-7.43 (m, 1H), 4.24-3.99 (m, 2H), 3.29-3.12 (m, 1H), 2.95-2.78 (m, 2H), 1.97-1.81 (m, 2H), 1.64-1.56 (m, 2H), 1.45 (s, 9H).

Step 3

To a solution tert-butyl 4-(4-cyano-2-fluorobenzoyl)piperidine-1-carboxylate (1.2 g, 3.61 mmol) in THE (10 mL) under nitrogen was added LiHMDS (7.22 mL, 1 M, 7.22 mmol) dropwise at −70° C. The mixture was stirred at −78° C. for 30 minutes. Then NFSI (1.36 g, 4.33 mmol) in THE (5 mL) was added dropwise. The mixture was stirred at −78° C. for 2 hours and was then poured into an aqueous NaHCO₃ solution (10 mL) and stirred for 20 minutes. The mixture was extracted with EtOAc (3×20 mL). The combined organic layers were washed with brine (2×10 mL), dried over anhydrous Na₂SO₄, filtered and purified by silica gel chromatography (PE/EtOAc=5/1 to 3/1) to afford tert-butyl 4-(4-cyano-2-fluorobenzoyl)-4-fluoropiperidine-1-carboxylate (550 mg, 43.6%). ¹H-NMR (400 MHz, CDCl₃) δ_{H}=7.57-7.51 (m, 2H), 7.48-7.43 (m, 1H), 4.21-4.01 (m, 2H), 3.09 (s, 2H), 2.17-1.93 (m, 4H), 1.48 (s, 9H).

Step 4

To a solution of tert-butyl 4-(4-cyano-2-fluorobenzoyl)-4-fluoropiperidine-1-carboxylate (400 mg, 1.14 mmol) in MeOH (10 mL) was slowly added NaBH₄ (86.6 mg, 2.28 mmol) in portions at 25° C. The mixture was stirred at 25° C. for 1 hour. An aqueous NH₄Cl solution (50 mL) was added and the mixture was extracted with EtOAc (2×20 mL). The combined organic layers were washed with brine (2×20 mL), dried over anhydrous Na₂SO₄, filtered and concentrated to give tert-butyl 4-((4-cyano-2-fluorophenyl)(hydroxy)methyl)-4-fluoropiperidine-1-carboxylate (250 mg, crude). ¹H-NMR (400 MHz, CDCl₃) δ_{H}=7.75-7.60 (m, 1H), 7.54-7.47 (m, 1H), 7.38-7.33 (m, 1H), 5.06 (d, J=15.2 Hz, 1H), 4.08-3.93 (m, 2H), 3.07-2.87 (m, 2H), 1.64-1.51 (m, 5H), 1.43 (s, 9H).

Step 5

To tert-butyl 4-((4-cyano-2-fluorophenyl)(hydroxy)methyl)-4-fluoropiperidine-1-carboxylate (250 mg, 0.709 mmol) in dioxane (10 mL) was added HCl/MeOH (10 mL, 4M, 40.0 mmol) at 15° C. The mixture was stirred at 15° C. for 16 hours under nitrogen. The mixture was concentrated to give 3-fluoro-4-((4-fluoropiperidin-4-yl)(hydroxy)methyl)benzonitrile hydrochloride (200 mg, crude), which was used directly in the next reaction.

Step 6

To a solution of 2-(pyrimidin-4-yl)nicotinic acid (153 mg, 0.762 mmol), HATU (524 mg, 1.38 mmol), DIPEA (0.602 mL, 3.46 mmol) and 3-fluoro-4-((4-fluoropiperidin-4-yl)(hydroxy)methyl)benzonitrile hydrochloride (200 mg, 0.917 mmol) in DMF (2 mL) was stirred at 15° C. for 16 hours. The mixture was poured into water (20 mL) and stirred for 20 minutes. The aqueous phase was extracted with EtOAc (30 mL). The combined organic layers were washed with brine (2×20 mL), dried over anhydrous Na₂SO₄, filtered and concentrated. The residue was purified by SFC (Column: DAICEL CHIRALPAK AS (250 mm*30 mm, 10 um), Condition A=0.1% NH₃H₂O B=EtOH, Begin B 25, End B 25, FlowRate (ml/min) 60) to afford racemic-3-fluoro-4-((4-fluoro-1-(2-(pyrimidin-4-yl)nicotinoyl)piperidin-4-yl)(hydroxy)methyl)benzonitrile (200 mg, 66.4%), which was further purified by SFC (Column: DAICEL CHIRALPAK AD (250 mm*30 mm, 10 um), Condition A=0.1% NH₃H₂O B=MEOH, Begin B 45, End B 45, Flow Rate (ml/min) 60) to afford:

(S)-3-fluoro-4-((4-fluoro-1-(2-(pyrimidin-4-yl)nicotinoyl)piperidin-4-yl)(hydroxy)methyl)benzonitrile (69.2 mg, 34.7%). ¹H-NMR (400 MHz, CDCl₃) δ_{H}=9.17 (s, 0.4H), 8.84 (br s, 1H), 8.75 (s, 1.6H), 8.33-8.15 (m, 1H), 7.74-7.61 (m, 2H), 7.56-7.42 (m, 2H), 7.41-7.31 (m, 1H), 5.20-5.02 (m, 1H), 4.87-4.54 (m, 1H), 3.59-2.50 (m, 4H), 2.34-1.84 (m, 3H). LCMS purity 100%, ee=100%, MS ESI calcd. for C₂₃H₂₀F₂N₅O₂[M+H]⁺ 436, found 436.

(R)-3-fluoro-4-((4-fluoro-1-(2-(pyrimidin-4-yl)nicotinoyl)piperidin-4-yl)(hydroxy)methyl)benzonitrile (62.3 mg, 31.3%). ¹H NMR (400 MHz, CDCl₃) δ_{H}=9.22-9.10 (m, 1H), 9.22-9.10 (m, 1H), 8.85 (s, 1H), 8.74 (s, 2H), 8.31-8.15 (m, 1H), 7.73-7.60 (m, 2H), 7.55-7.41 (m, 2H), 7.41-7.31 (m, 1H), 5.18-5.04 (m, 1H), 4.86-4.56 (m, 1H), 3.60-2.65 (m, 3H), 2.30-1.82 (m, 3H). LCMS purity 100%, ee=99.8%. MS ESI calcd. for C₂₃H₂₀F₂N₅O₂[M+H]⁺ 436, found 436.

### Example 19. Synthesis of 3-fluoro-4-(4-fluoro-1-(2-(pyrimidin-4-yl)nicotinoyl)piperidine-4-carbonyl)benzonitrile (Cmpd 17)

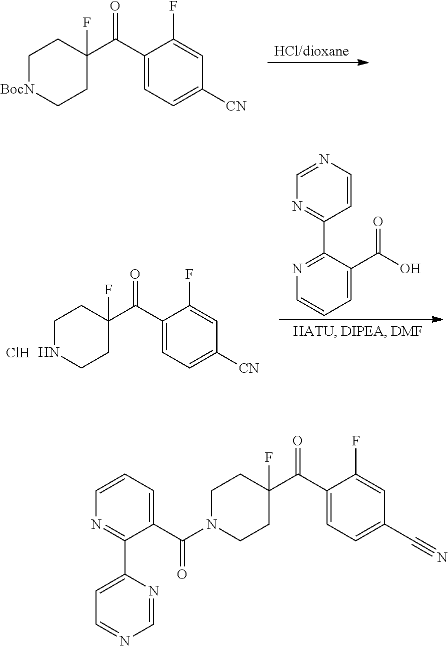 [SMILES: CC(C)(C)OC(=O)N1CCC(F)(C(=O)c2ccc(C#N)cc2F)CC1 ; Cl ; N#Cc1ccc(C(=O)C2(F)CCNCC2)c(F)c1 ; O=C(O)c1cccnc1-c1ccncn1 ; N#Cc1ccc(C(=O)C2(F)CCN(C(=O)c3cccnc3-c3ccncn3)CC2)c(F)c1]

Step 1

To a solution of tert-butyl 4-(4-cyano-2-fluorobenzoyl)-4-fluoropiperidine-1-carboxylate (150 mg, 0.428 mmol) in ??? was added HCl/MeOH (10 mL, 4M, 40.0 mmol) at 15° C. The mixture was stirred at 15° C. for 16 hour under nitrogen. The mixture was concentrated to give 3-fluoro-4-(4-fluoropiperidine-4-carbonyl)benzonitrile hydrochloride (120 mg, crude). The crude residue was used directly in the next step.

Step 2

A mixture of 2-(pyrimidin-4-yl)nicotinic acid (92.6 mg, 0.46 mmol), HATU (318 mg, 0.84 mmol), DIPEA (0.363 mL, 2.09 mmol) and 3-fluoro-4-(4-fluoropiperidine-4-carbonyl)benzonitrile hydrochloride (120 mg, 0.418 mmol) in DMF (10 mL) was stirred at 15° C. for 16 hours. The mixture was poured into water (20 mL) and stirred for 20 minutes. The aqueous phase was extracted with EtOAc (30 mL). The combined organic phases were washed with brine (3×20 mL), dried over anhydrous Na₂SO₄, filtered and concentrated. The residue was purified by prep-HPLC (Column: Phenomenex Gemini-NX 80*30 mm*3 um, Condition A=water (10 mM NH₄HCO₃)-B=ACN, Begin B 27, End B 57, Flow Rate (ml/min) 60) to afford 3-fluoro-4-(4-fluoro-1-(2-(pyrimidin-4-yl)nicotinoyl)piperidine-4-carbonyl)benzonitrile (82.6 mg, 45.6%). ¹H-NMR (400 MHz, CDCl₃)^{6H}=9.43 (s, 0.6H), 9.19 (s, 0.4H), 8.97-8.84 (m, 1H), 8.81-8.71 (m, 1H), 8.36-8.17 (m, 1H), 7.82-7.64 (m, 1H), 7.61-7.53 (m, 2H), 7.52-7.43 (m, 2H), 4.92-4.62 (m, 1H), 3.77-3.51 (m, 1H), 3.31-3.05 (m, 2H), 2.81-2.12 (m, 3H), 1.94-1.76 (m, 1H). LCMS purity 100%, MS ESI calcd. For C₂₃H₁₈F₂NsO₂ [M+H]⁺ 434, found 434. ¹⁹F-NMR (400 MHz, CDCl₃) δ_{F}−106.0, −162.

### Example 20. Synthesis of (4-((5-chloropyridin-2-yl)methyl)-4-fluoropiperidin-1-yl)(2-(pyrimidin-4-yl)pyridin-3-yl)methanone (Cmpd 18)

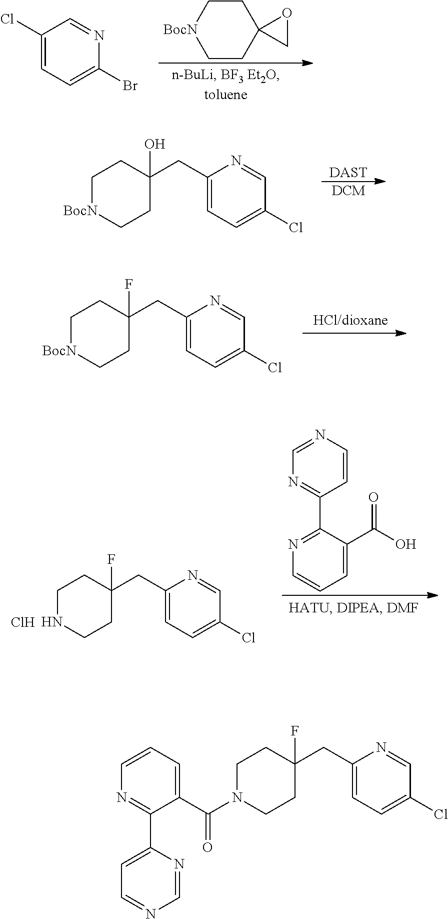

Step 1

To a solution of 2-bromo-5-chloropyridine (2 g, 10.3 mmol) in toluene (20 mL) at −78° C. under nitrogen was added slowly a solution of n-BuLi (8.24 mL, 2.5M in hexane, 20.6 mmol), and the mixture was stirred at −78° C. for 30 minutes. A solution of tert-butyl 1-oxa-6-azaspiro[2.5]octane-6-carboxylate (2.43 g, 11.4 mmol) in toluene (10 mL) and BF₃ Et₂O (1.47 g, 10.4 mmol) was added dropwise and the resulting mixture was stirred at −70° C. for 2 hours. The mixture was poured slowly into saturated aqueous NH₄Cl solution (200 mL) and the mixture was extracted with EtOAc (3×100 mL). The combined organic layers were washed with brine (2×100 mL), dried over anhydrous Na₂SO₄, and concentrated to give tert-butyl 4-((5-chloropyridin-2-yl)methyl)-4-hydroxypiperidine-1-carboxylate (400 mg, impure). ¹H-NMR (400 MHz, CDCl₃) δ_{H} 8.47 (d, J=2.4 Hz, 1H), 7.62 (dd, J=8.4, 2.4 Hz, 1H), 7.08 (d, J=8.4 Hz, 1H), 5.27 (br s, 1H), 3.67-3.89 (m, 2H), 3.21 (br s, 2H), 2.88 (s, 2H), 1.56-1.25 (m, 13H).

Step 2

To a solution of tert-butyl 4-((5-chloropyridin-2-yl)methyl)-4-hydroxypiperidine-1-carboxylate (400 mg, 1.22 mmol) in DCM (10 mL) was added DAST (393 mg, 2.44 mmol) at 0° C. The mixture was stirred at 0° C. for 30 minutes. The mixture was poured into ice-cold NaHCO₃ (80 mL) and stirred for 20 minutes. The mixture was extracted with DCM (3×30 mL). The combined organic layers were washed with brine (2×20 mL), dried over anhydrous Na₂SO₄, filtered and concentrated to give tert-butyl 4-((5-chloropyridin-2-yl)methyl)-4-fluoropiperidine-1-carboxylate (400 mg, crude). ¹H-NMR (400 MHz, CDCl₃) δ_{H} 8.50 (d, J=2.3 Hz, 1H), 7.60 (td, J=7.9, 2.5 Hz, 1H), 7.06-7.20 (m, 1H), 5.30 (s, 1H), 3.72-4.00 (m, 2H), 3.33-3.53 (m, 2H), 2.99-3.16 (m, 2H), 1.45 (d, J=3.0 Hz, 12H). ¹⁹F-NMR (376.5 MHz, CDCl₃) δ_{F} −160.379.

Step 3

To a solution of tert-butyl 4-((5-chloropyridin-2-yl)methyl)-4-fluoropiperidine-1-carboxylate (400 mg, 1.21 mmol) in dioxane (5 mL) was added HCl/dioxane (1.51 mL, 4M, 6.05 mmol) and the mixture was stirred at 25° C. for 4 hours. The mixture was concentrated to give 5-chloro-2-((4-fluoropiperidin-4-yl)methyl)pyridine hydrochloride (400 mg, crude), which was used directly in the next reaction.

Step 4

A mixture of 2-(pyrimidin-4-yl)nicotinic acid (301 mg, 1.5 mmol), HATU (855 mg, 2.25 mmol), DIPEA (969 mg, 7.5 mmol) and 5-chloro-2-((4-fluoropiperidin-4-yl)methyl)pyridine hydrochloride (400 mg, 1.5 mmol) in DMF (5 mL) was stirred at 25° C. for 2 hours. The mixture was poured into water (20 mL) and extracted with EtOAc (3×10 mL). The combined organic layers were washed with brine (2×20 mL), dried over anhydrous Na₂SO₄, filtered and concentrated. The residue was purified by prep-HPLC (Column: Phenomenex Gemini-NX 80*40 mm*3 μm Condition A=water (0.05% NH₃H₂O)-B=ACN Begin B 18 End B 48) to afford (4-((5-chloropyridin-2-yl)methyl)-4-fluoropiperidin-1-yl)(2-(pyrimidin-4-yl)pyridin-3-yl)methanone (50.1 mg, 8%). ¹H NMR (400 MHz, CDCl₃) δ_{H} 9.18-8.97 (m, 2H), 8.74 (d, J=3.2 Hz, 1H), 8.49 (br s, 1H), 8.24 (br d, J=5.2 Hz, 1H), 7.54-7.73 (m, 2H), 7.45 (br s, 1H), 7.22 (br t, J=8.4 Hz, 1H), 4.42-4.72 (m, 1H), 3.33-3.54 (m, 1H), 2.98-3.29 (m, 4H), 1.64-2.06 (m, 4H). LC-ELSD/MS purity 99%; MS ESI calcd. for C₂₁H₂₀ClFN₅O [M+H]⁺ 412.1, found 412.1. ¹⁹F-NMR (376.5 MHz, CDCl₃) δ_{F} −160.064.

### Example 21. Synthesis of (4-(2,4-difluorobenzyl)-4-fluoropiperidin-1-yl)(2-(pyrimidin-4-yl)pyridin-3-yl)methanone (Cmpd 19)

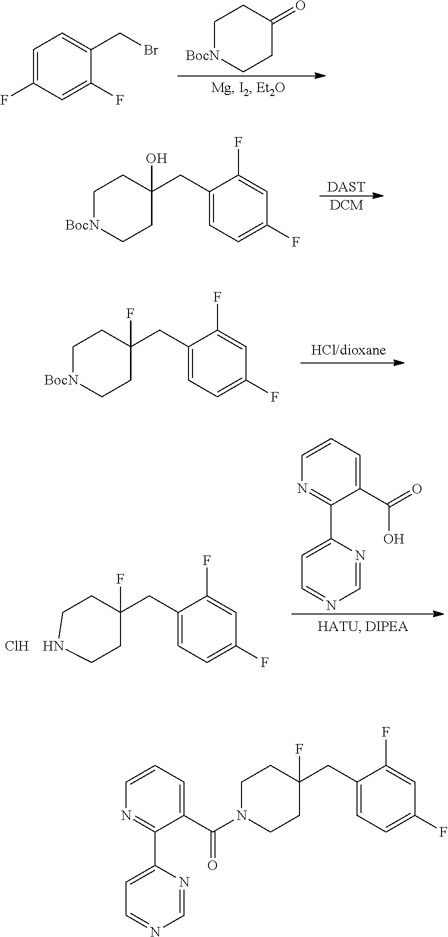

Step 1

To a mixture of Mg (0.46 g, 19.3 mmol) and I₂ (2.45 mg, 0.00966 mmol) in THF (5 mL) was added 1-(bromomethyl)-2,4-difluorobenzene (2.0 g, 9.66 mmol) in THE (5 mL) at 25° C. under nitrogen. The mixture was heated to 50° C. and stirred for 1 hour. The mixture was cooled to room temperature and then added to a solution of tert-butyl 4-oxopiperidine-1-carboxylate (1.98 g, 9.94 mmol) in THF (10 mL) at 25° C. The mixture was stirred at 25° C. for 1 hour. The mixture was poured into an aqueous NH₄Cl solution (100 mL) and stirred for 1 hour. The mixture was extracted with EtOAc (3×50 mL). The combined organic layers were washed with brine (2×100 mL), dried over Na₂SO₄, filtered and concentrated. The residue was purified by silica gel chromatography (10˜40% of EtOAc in PE) to give tert-butyl 4-(2,4-difluorobenzyl)-4-hydroxypiperidine-1-carboxylate (720 mg, 22.1%). ¹H NMR (400 MHz, CDCl₃) δ_{H} 8.39 (m, 1H), 7.44-7.35 (m, 1H), 7.13 (m, 1H), 3.80 (s, 2H), 3.23 (s, 2H), 1.58 (m, 3H), 1.52-1.48 (m, 2H), 1.46 (s, 9H).

Step 2

To a mixture of tert-butyl 4-(2,4-difluorobenzyl)-4-hydroxypiperidine-1-carboxylate (400 mg, 1.22 mmol) in DCM (5 mL) was added DAST (0.322 mL, 2.44 mmol) at 0° C. The mixture was stirred at 20° C. for 0.5 hours. The mixture was poured into water (50 mL) and stirred for 20 minutes. The mixture was extracted with DCM (3×20 mL). The combined organic layers were washed with brine (2×50 mL), dried over anhydrous Na₂SO₄, filtered and concentrated to give tert-butyl 4-(2,4-difluorobenzyl)-4-fluoropiperidine-1-carboxylate (400 mg, crude), which was used directly in the next reaction.

Step 3

To a solution of tert-butyl 4-(2,4-difluorobenzyl)-4-fluoropiperidine-1-carboxylate (0.4 g, 1.21 mmol) in dioxane (5 mL) was added HCl/dioxane (5 mL, 4M in dioxane, 18.1 mmol), and the mixture was stirred at 25° C. for 4 hours. The mixture concentrated to give 4-(2,4-difluorobenzyl)-4-fluoropiperidine hydrochloride (350 mg, crude), which was used directly in the next reaction.

Step 4

To a mixture of 2-(pyrimidin-4-yl)nicotinic acid (263 mg, 1.31 mmol), 4-(2,4-difluorobenzyl)-4-fluoropiperidine hydrochloride (350 mg, 1.31 mmol) and HATU (745 mg, 1.96 mmol) in DMF (5 mL) was added DIPEA (0.68 mL, 3.93 mmol). The mixture was stirred at 20° C. for 12 hours. The reaction mixture poured into H₂O (50 mL) and stirred for 20 minutes. The mixture was extracted with EtOAc (3×20 mL). The combined organic layers were washed with brine (2×20 mL), dried over anhydrous Na₂SO₄, filtered and concentrated. The residue was purified by silica gel chromatography (0˜10% of MeOH in DCM) and further purified by SFC (Column DAICEL CHIRALCEL OD-H (250 mm*30 mm, 5 um) Condition A=0.1% NH₃H₂O B=EtOH Begin B 30% End B 30% Gradient Time(min) 100% B Hold Time(min) Flow Rate (ml/min) 60 Injections 100) to give (4-(2,4-difluorobenzyl)-4-fluoropiperidin-1-yl)(2-(pyrimidin-4-yl)pyridin-3-yl)methanone (107 mg, 20%). ¹H-NMR (400 MHz, CDCl₃) δ_{H} 9.19 (s, 1H), 8.91-8.81 (m, 1H), 8.75 (dd, J=1.4, 4.8 Hz, 1H), 8.33-8.19 (m, 1H), 7.75-7.63 (m, 1H), 7.51-7.41 (m, 1H), 7.26-7.17 (m, 1H), 6.93-6.77 (m, 2H), 4.75-4.51 (m, 1H), 3.53-3.35 (m, 1H), 3.31-3.09 (m, 2H), 3.03-2.88 (m, 2H), 2.09-1.81 (m, 2H), 1.73-1.61 (m, 1H). ¹⁹F-NMR (400 MHz, CDCl₃) δ_{F} −111.176, 160.979. LC-ELSD/MS purity ≥98%, MS ESI calcd. for C₂₂H₁₉F₃N₄O [M+H]⁺ 413.2, found 413.2.

### Example 22. Synthesis of (4-(3,5-difluorobenzyl)-4-fluoropiperidin-1-yl)(2-(pyrimidin-4-yl)pyridin-3-yl)methanone (Cmpd 20)

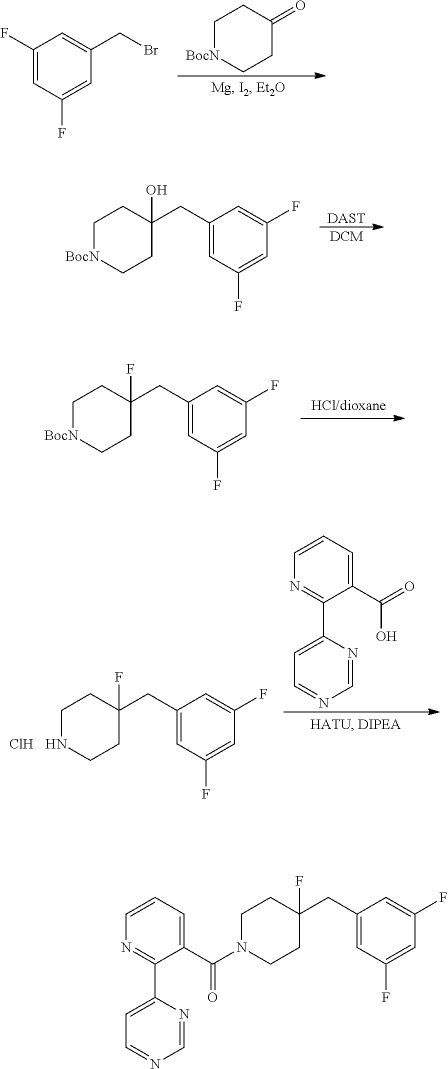

Step 1

To a mixture of Mg (0.56 g, 23.2 mmol) and I₂ (2.94 mg, 0.0116 mmol) in Et₂O (10 mL) was added 1-(bromomethyl)-3,5-difluorobenzene (3.0 g, 11.6 mmol) in Et₂O (10 mL) at 25° C. under nitrogen. The mixture was stirred at 50° C. for 1 hour. The mixture was cooled and added dropwise to a solution of tert-butyl 4-oxopiperidine (1.98 g, 9.94 mmol) in THF at 25° C. and stirred for 1 hour. The mixture was poured into an aqueous NH₄Cl solution (50 mL) and stirred for 0.5 hours. The mixture was extracted with EtOAc (50 mL×3), and the combined organic layers were washed with brine (2×10 mL), dried over Na₂SO₄, filtered and concentrated. The residue was purified by silica gel chromatography (10˜40% of EtOAc in PE) to give tert-butyl 4-(3,5-difluorobenzyl)-4-hydroxypiperidine-1-carboxylate (600 mg, 18%). ¹H-NMR (400 MHz, CDCl₃) δ_{H} 6.82-6.66 (m, 3H), 3.86 (br s, 2H), 3.10 (m, 2H), 2.74 (s, 2H), 1.65-1.50 (m, 4H), 1.46 (s, 9H).

Step 2

To a mixture of tert-butyl 4-(3,5-difluorobenzyl)-4-hydroxypiperidine-1-carboxylate (600 mg, 1.83 mmol) in DCM (5 mL) was added DAST (0.482 mL, 3.66 mmol) at 0° C. and the mixture was stirred at 20° C. for 0.5 hours. The mixture was poured into water (50 mL) and stirred for 20 minutes. The mixture was extracted with DCM (3×20 mL), and the combined organic layers were washed with brine (2×50 mL), dried over anhydrous Na₂SO₄, filtered and concentrated to give tert-butyl 4-(3,5-difluorobenzyl)-4-fluoropiperidine-1-carboxylate (600 mg, crude), which was used directly in the next reaction.

Step 3

To a mixture of tert-butyl 4-(3,5-difluorobenzyl)-4-fluoropiperidine-1-carboxylate (0.4 g, 1.21 mmol) in dioxane (5 mL) was added HCl/dioxane (3.0 mL, 4M in dioxane, 12.1 mmol), the mixture was stirred at 25° C. for 4 h. The mixture was cooled and concentrated to give 4-(3,5-difluorobenzyl)-4-fluoropiperidine hydrochloride (600 mg, crude), which was used directly in the next reaction.

Step 4

To a solution of 2-(pyrimidin-4-yl)nicotinic acid (340 mg, 1.69 mmol), 4-(3,5-difluorobenzyl)-4-fluoropiperidine hydrochloride (450 mg, 1.69 mmol) and HATU (962 mg, 2.53 mmol) in DMF (5 mL) was added DIPEA (0.88 mL, 5.06 mmol). The mixture was stirred at 20° C. for 12 hours. The mixture was poured into H₂O (50 mL) and stirred for 20 minutes. The mixture was extracted with EtOAc (3×20 mL), and the combined organic layers were washed with brine (2×20 mL), dried over anhydrous Na₂SO₄, filtered and concentrated. The residue was purified silica gel chromatography (0˜10% of MeOH in DCM) to afford the desired product (700 mg, impure), which was further purified by SFC (Column DAICEL CHIRALCEL OD-H (250 mm*30 mm, 5 um) Condition A=0.1% NH₃H₂O B=EtOH Begin B 30% End B 30% Gradient Time(min) 100% B Hold Time(min) Flow Rate (ml/min) 60 Injections 100) to give (4-(3,5-difluorobenzyl)-4-fluoropiperidin-1-yl)(2-(pyrimidin-4-yl)pyridin-3-yl)methanone (234 mg, 33.6%). ¹H-NMR (400 MHz, CDCl₃) δ_{H} 9.12 (s, 1H), 8.87-8.75 (m, 1H), 8.68 (m, 1H), 8.17 (d, J=4.6 Hz, 1H), 7.68-7.56 (m, 1H), 7.38 (m, 1H), 6.66 (d, J=5.4 Hz, 3H), 4.72-4.43 (m, 1H), 3.44-3.27 (m, 1H), 3.20-3.00 (m, 2H), 2.95-2.72 (m, 2H), 1.97-1.57 (m, 3H). ¹⁹F-NMR (400 MHz, CDCl₃) δ_{F} −110.005, −161.317. LC-ELSD/MS purity ≥99%, MS ESI calcd. for C₂₂H₁₉F₃N₄O [M+H]⁺ 413.2, found 413.2.

### Example 23. Synthesis of 6-((4-fluoro-1-(2-(pyrimidin-4-yl)nicotinoyl)piperidin-4-yl)methyl)nicotinonitrile (Cmpd 21)

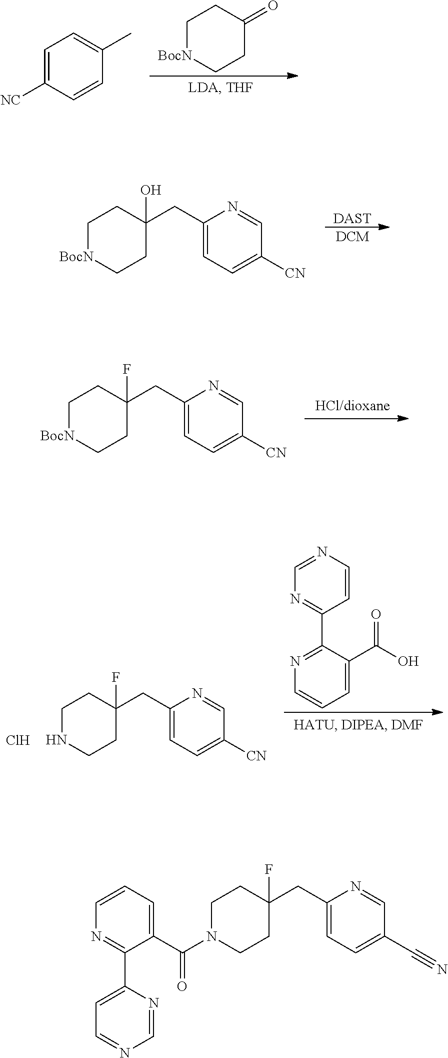

Step 1

To a mixture of 6-methylnicotinonitrile (1 g, 8.46 mmol) in THF (15 mL) was added dropwise LDA (6.3 mL, 2 M in THF, 12.6 mmol) at −78° C. and the mixture was stirred at −78° C. for 1 hour under nitrogen. Then tert-butyl 4-oxopiperidine-1-carboxylate (2.51 g, 12.6 mmol) was added and stirring was continued at −70° C. for 2 hours. The mixture was poured into water (100 mL) and extracted with EtOAc (3×50 mL). The combined organic layers were washed with brine (2×30 mL), dried over Na₂SO₄, filtered and concentrated. The residue was purified by prep-HPLC (Column: Phenomenex Gemini-NX 80*40 mm*3 um Condition A=water (0.05% NH3H2O)-B=ACN Begin B 24End B 54) to give tert-butyl 4-((5-cyanopyridin-2-yl)methyl)-4-hydroxypiperidine-1-carboxylate (100 mg, impure). ¹H-NMR (400 MHz, CDCl3) δ_{H} ppm 8.80 (d, J=2.0 Hz, 1H), 7.92 (dd, J=8.0, 2.0 Hz, 1H), 7.29 (br d, J=8.0 Hz, 1H), 4.81 (br s, 1H), 3.81 (br s, 2H), 3.20 (br s, 2H), 3.00 (s, 2H), 1.48-1.53 (m, 4H), 1.45 (s, 9H).

Step 2

To a solution of tert-butyl 4-((5-cyanopyridin-2-yl)methyl)-4-hydroxypiperidine-1-carboxylate (100 mg, 0.3150 mmol) in DCM (10 mL) was added DAST (101 mg, 0.63 mmol) at 0° C. The mixture was stirred at 0° C. for 5 minutes. The reaction mixture was poured into ice-water and NaHCO₃ (80 ml) and stirred for 20 min. The mixture was extracted with DCM (3×30 mL). The combined organic layers were washed with brine (2×20 mL), dried over anhydrous Na₂SO₄, filtered and concentrated to give tert-butyl 4-((5-cyanopyridin-2-yl)methyl)-4-fluoropiperidine-1-carboxylate (100 mg, crude), which was used directly in the next reaction.

Step 3

To a solution of tert-butyl 4-((5-cyanopyridin-2-yl)methyl)-4-fluoropiperidine-1-carboxylate (100 mg, 0.3131 mmol) in dioxane (10 mL) was added HCl/dioxane (0.39 mL, 4 M in dioxane, 1.56 mmol) and the mixture was stirred at 25° C. for 2 h. The mixture was cooled and concentrated to give 6-((4-fluoropiperidin-4-yl)methyl)nicotinonitrile hydrochloride (100 mg, crude), which was used directly in the next reaction.

Step 4

2-(Pyrimidin-4-yl)nicotinic acid (117 mg, 0.5865 mmol), HATU (334 mg, 0.8797 mmol), DIPEA (378 mg, 2.93 mmol) and 6-((4-fluoropiperidin-4-yl)methyl)nicotinonitrile hydrochloride (150 mg, 0.5865 mmol) were combined in DMF (5 mL) and stirred at 25° C. for 2 hours. The mixture was poured into water (20 mL) and extracted with EtOAc (3×10 mL). The combined organic layers were washed with brine (2×20 mL), dried over anhydrous Na₂SO₄, filtered, and concentrated. The residue was purified by prep-HPLC (Column: Phenomenex Gemini-NX 80*40 mm*3 um Condition A=water (0.05% NH₃H₂O)-B=ACN Begin B 14 End B) to afford 6-((4-fluoro-1-(2-(pyrimidin-4-yl)nicotinoyl)piperidin-4-yl)methyl)nicotinonitrile (38.7 mg, 16%). ¹H-NMR (400 MHz, CDCl₃) δ_{H} 9.18-9.05 (s, 1H), 8.88 (br dd, J=13.2, 4.4 Hz, 1H), 8.72-8.83 (m, 2H), 8.25 (br s, 1H), 7.91 (dd, J=8.0, 2.0 Hz, 1H), 7.60-7.74 (m, 1H), 7.36-7.50 (m, 2H), 4.32-4.80 (m, 1H), 3.40 (br d, J=15.2 Hz, 1H), 3.11-3.30 (m, 4H), 1.88-2.10 (m, 2H), 1.67 (br d, J=17.2 Hz, 2H). LC-ELSD/MS purity 100%; MS ESI calcd. for C₂₂H₂₀FN₆O [M+H]⁺ 403.2, found 403.2. ¹⁹F-NMR (376.5 MHz, CDCl₃) δ_{F} −160.607.

### Example 24. Synthesis of (4-fluoro-4-((5-(trifluoromethyl)pyridin-2-yl)methyl)piperidin-1-yl)(2-(pyrimidin-4-yl)pyridin-3-yl)methanone (Cmpd 22)

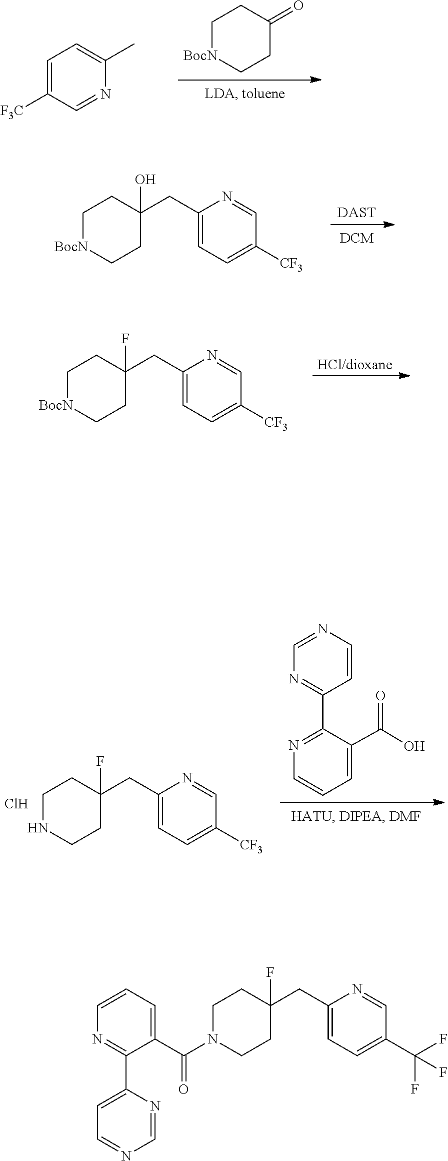

Step 1

To a solution of 2-methyl-5-(trifluoromethyl)pyridine (2.0 g, 12.4 mmol) in THF (15 mL) was added dropwise LDA (18.6 mL, 2 M in THF, 37.2 mmol) at −78° C., and the mixture was stirred at −78° C. for 1 hour. Then tert-butyl 4-oxopiperidine-1-carboxylate (2.47 g, 12.4 mmol) was added, and the mixture was stirred at −70° C. for 2 hours. The mixture was poured into water (100 mL) and was extracted with EtOAc (3×100 mL). The combined organic layers were washed with brine (2×50 mL), dried over anhydrous Na₂SO₄, filtered and concentrated. The residue was purified by prep-HPLC (Column: Phenomenex Gemini NX C18 150*40 mm*5 μm Condition A=water (0.04% NH₃H₂O+10 mM NH₄HCO₃)-B=ACN Begin B 32 End B 62) to give tert-butyl 4-hydroxy-4-((5-(trifluoromethyl)pyridin-2-yl)methyl)piperidine-1-carboxylate (280 mg, 6.27%). ¹H-NMR (400 MHz, CDCl₃) δ_{H} 8.79 (s, 1H), 7.89 (m, 1H), 7.28 (s, 1H), 5.23 (s, 1H), 3.71-3.93 (m, 2H), 3.14-3.31 (m, 2H), 2.99 (s, 2H), 1.48-1.63 (m, 10H), 1.45 (s, 10H).

Step 2

To a solution of tert-butyl 4-hydroxy-4-((5-(trifluoromethyl)pyridin-2-yl)methyl)piperidine-1-carboxylate (280 mg, 0.7769 mmol) in DCM (10 mL) was added DAST (249 mg, 1.55 mmol) at 0° C., and the mixture was stirred at 0° C. for 5 minutes. The mixture was poured into an aqueous NaHCO₃ solution (80 mL) and stirred for 20 minutes. The mixture was extracted with DCM (3×30 mL), and the combined organic layers were washed with brine (2×20 mL), dried over anhydrous Na₂SO₄, filtered and concentrated to give tert-butyl 4-fluoro-4-((5-(trifluoromethyl)pyridin-2-yl)methyl)piperidine-1-carboxylate (280 mg, crude), which was used directly in the next reaction.

Step 3

To a solution of tert-butyl 4-fluoro-4-((5-(trifluoromethyl)pyridin-2-yl)methyl)piperidine-1-carboxylate (280 mg, 0.7726 mmol) in dioxane (10 mL) was added HCl/dioxane (0.965 mL, 4M in dioxane, 3.86 mmol), and the mixture was stirred at 25° C. for 2 hours. The mixture was concentrated to give 2-((4-fluoropiperidin-4-yl)methyl)-5-(trifluoromethyl)pyridine hydrochloride (280 mg, crude), which was used directly in the next reaction.

Step 4

2-(Pyrimidin-4-yl)nicotinic acid (188 mg, 0.9373 mmol), HATU (532 mg, 1.40 mmol), DIPEA (604 mg, 4.68 mmol) and 2-((4-fluoropiperidin-4-yl)methyl)-5-(trifluoromethyl)pyridine hydrochloride (280 mg, 0.9373 mmol) were combined in DMF (5 mL) and stirred at 25° C. for 2 hours. The mixture was poured into water (30 mL) and extracted with EtOAc (3×15 mL). The combined organic layers were washed with brine (2×10 mL), dried over anhydrous Na₂SO₄, filtered and concentrated. The residue was purified by prep-HPLC (Column: Phenomenex Gemini-NX 80*40 mm*3 μm Condition A=water (0.05% NH₃H₂O)-B=ACN Begin B 25End B 55) to afford (4-fluoro-4-((5-(trifluoromethyl)pyridin-2-yl)methyl)piperidin-1-yl)(2-(pyrimidin-4-yl)pyridin-3-yl)methanone (72.4 mg, 17.3%). ¹H-NMR (400 MHz, CDCl₃) δ_{H} 9.18-9.03 (s, 1H), 8.87 (d, J=12.0, 4.0 Hz, 1H), 8.72-8.81 (m, 2H), 8.18-8.30 (m, 1H), 7.88 (d, J=8.0, 2.0 Hz, 1H), 7.63-7.72 (m, 1H), 7.34-7.48 (m, 2H), 7.34-7.48 (m, 1H), 4.42-4.78 (m, 1H), 3.33-3.52 (m, 1H), 3.12-3.31 (m, 4H), 1.76-2.25 (m, 3H). LC-ELSD/MS purity 95%; MS ESI calcd. for C₂₂H₂₀F₄N₅O [M+H]⁺ 446.2, found 446.2. ¹⁹F-NMR (376.5 MHz, CDCl₃) δ_{F} −62.355; −160.521.

### Example 25. Synthesis of (4-fluoro-4-((6-(trifluoromethyl)pyridin-3-yl)methyl)piperidin-1-yl)(2-(pyrimidin-4-yl)pyridin-3-yl)methanone (Cmpd 23)

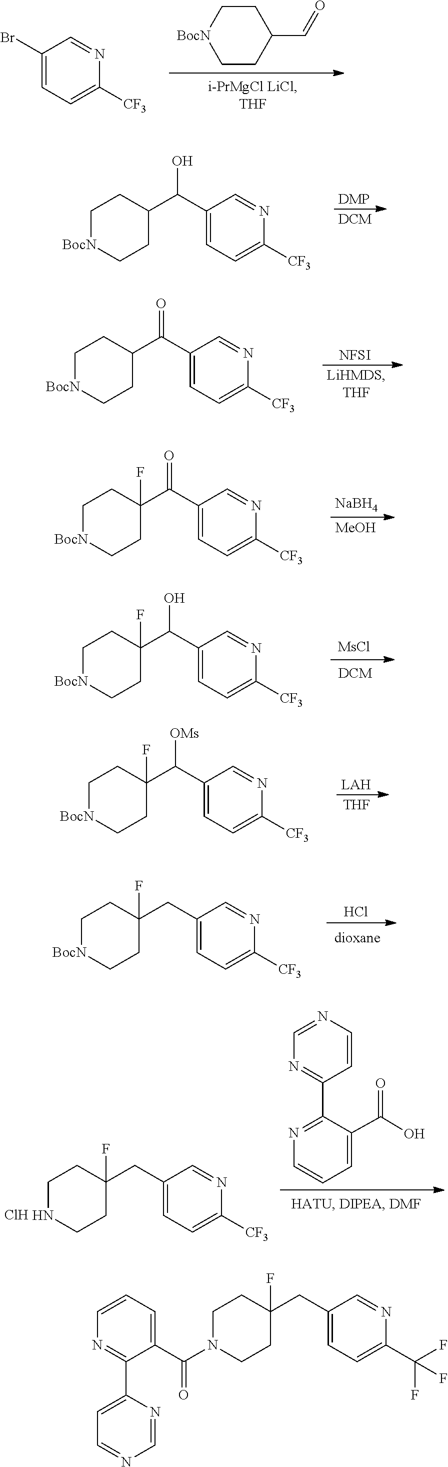

Step 1

To a solution of 5-bromo-2-(trifluoromethyl)pyridine (5.0 g, 22.1 mmol) in THE (10 mL) was added slowly a solution of iPrMgCl LiCl (17.0 mL, 1.3 M in hexane, 22.1 mmol) at 0° C., and the mixture was stirred at 0° C. for 30 minutes. To this mixture was added a solution of tert-butyl 4-formylpiperidine-1-carboxylate (4.64 g, 21.8 mmol) in THE (5 mL) dropwise at 0° C. under nitrogen. After stirring at 0° C. for 1 hour, the mixture was poured into water (100 mL) and stirred for 20 minutes and then was extracted with EtOAc (3×20 mL). The combined organic layers were washed with brine (2×100 mL), dried over anhydrous Na₂SO₄, filtered and concentrated. The residue was purified by silica gel chromatography (10˜30% of EtOAc in PE) to give tert-butyl 4-(hydroxy(6-(trifluoromethyl)pyridin-3-yl)methyl)piperidine-1-carboxylate (5.7 g, 72.6%). ¹H-NMR (400 MHz, CDCl₃) δ_{H} 8.58 (s, 1H), 7.79 (d, J=6.5 Hz, 1H), 7.62 (d, J=8.0 Hz, 1H), 4.53 (m, 1H), 4.06 (s, 2H), 2.55 (d, J=14.1 Hz, 2H), 2.13 (d, J=3.0 Hz, 1H), 1.82-1.63 (m, 2H), 1.42-1.34 (m, 11H), 1.29-1.17 (m, 3H).

Step 2

To a solution of tert-butyl 4-(hydroxy(6-(trifluoromethyl)pyridin-3-yl)methyl)piperidine-1-carboxylate (5.6 g, 15.5 mmol) in DCM (50 mL) was added DMP (13.1 g, 31.0 mmol) in portions at 25° C. The reaction mixture was stirred at 25° C. for 0.5 hour and then poured into a saturated aqueous solution of NaHCO₃/Na₂S₂O₃ (1:1) (200 mL). The mixture was extracted with DCM (2×50 mL), and the combined organic layers were washed with saturated aqueous NaHCO₃/Na₂S₂O₃ (1:1) (2×100 mL), dried over anhydrous Na₂SO₄, filtered and concentrated. The residue was purified by silica gel chromatography (0˜30% of EtOAc in PE) to give tert-butyl 4-(6-(trifluoromethyl)nicotinoyl)piperidine-1-carboxylate (6.0 g, impure). ¹H-NMR (400 MHz, CDCl₃) δ_{H} 9.21 (d, J=1.5 Hz, 1H), 8.39 (m, 1H), 8.25 (m, 1H), 8.06-7.90 (m, 1H), 7.82 (d, J=8.3 Hz, 1H), 7.75-7.69 (m, 1H), 4.17 (d, J=9.0 Hz, 2H), 3.38 (m, 1H), 2.93 (m, 2H), 2.26 (s, 1H), 1.87 (d, J=12.3 Hz, 2H), 1.77-1.65 (m, 3H), 1.46 (s, 9H).

Step 3

To a solution tert-butyl 4-(6-(trifluoromethyl)nicotinoyl)piperidine-1-carboxylate (6.0 g, 16.7 mmol) in THE (50 mL) was added LiHMDS (25.0 mL, 25.0 mmol) dropwise at −78° C. The mixture was stirred at −78° C. for 30 minutes. A solution of NFSI (6.30 g, 20.0 mmol) in THF (10 mL) was added dropwise at −78° C. The mixture was poured into NaHCO₃ solution (100 mL), stirred for 20 minutes, and then extracted with EtOAc (3×20 mL). The combined organic layers were washed with saturated brine (2×100 mL), dried over anhydrous Na₂SO₄, filtered and concentrated. The residue was purified by silica gel chromatography (0˜30% of EtOAc in PE) to afford tert-butyl 4-fluoro-4-(6-(trifluoromethyl)nicotinoyl)piperidine-1-carboxylate (4.7 g, 74.8%). ¹H-NMR (400 MHz, CDCl₃) δ_{H} 9.33 (s, 1H), 8.49 (d, J=7.5 Hz, 1H), 7.81 (d, J=8.3 Hz, 1H), 4.23-4.01 (m, 2H), 3.29-3.12 (m, 2H), 2.25-2.00 (m, 4H), 1.64 (s, 2H), 1.49 (s, 9H).

Step 4

To a solution of tert-butyl 4-fluoro-4-(6-(trifluoromethyl)nicotinoyl)piperidine-1-carboxylate (1.0 g, 2.65 mmol) in MeOH (20 mL) was slowly added NaBH₄ (0.2 g, 5.30 mmol) in portions at 25° C. The mixture was stirred at 25° C. for 1 hour and then a saturated aqueous solution of Na₂S₂O₃ (200 mL) was added and the resulting mixture was stirred for 30 minutes. The mixture was extracted with DCM (2×50 mL), and the combined organic layers were washed with brine (2×20 mL), dried over anhydrous Na₂SO₄, filtered and concentrated to give tert-butyl 4-fluoro-4-(hydroxy(6-(trifluoromethyl)pyridin-3-yl)methyl)piperidine-1-carboxylate (1.0 g, impure), which was used directly in the next reaction.

Step 5

To a solution of tert-butyl 4-fluoro-4-(hydroxy(6-(trifluoromethyl)pyridin-3-yl)methyl)piperidine-1-carboxylate (1.0 g, 2.64 mmol) in DCM (10 mL) was added TEA (1.1 mL, 7.92 mmol) and methanesulfonyl chloride (0.6 g, 5.28 mmol) in one portion at 0° C. The mixture was stirred at 0° C. for 2 hours and then slowly poured into H₂O (50 mL). The mixture was extracted with DCM (3×20 mL), and the combined organic layers were washed with brine (2×10 mL), dried over anhydrous Na₂SO₄, filtered and concentrated to give tert-butyl 4-fluoro-4-(((methylsulfonyl)oxy)(6-(trifluoromethyl)pyridin-3-yl)methyl)piperidine-1-carboxylate (1.3 g, crude). ¹H-NMR (400 MHz, CDCl₃) δ 8.75 (s, 1H), 8.26-8.16 (m, 1H), 7.97 (d, J=8.0 Hz, 1H), 7.77 (d, J=8.3 Hz, 1H), 6.53-6.46 (m, 1H), 5.50 (d, J=17.6 Hz, 1H), 4.07 (s, 2H), 2.98 (s, 3H), 1.81-1.57 (m, 6H), 1.46 (s, 9H).

Step 6

To a solution of tert-butyl 4-fluoro-4-(((methylsulfonyl)oxy)(6-(trifluoromethyl)pyridin-3-yl)methyl)piperidine-1-carboxylate (1.3 g, 2.84 mmol) in THE (5 mL) was added LAH (215 mg, 5.68 mmol). After stirring at 50° C. for 2 hours, the mixture was diluted with water (30 mL) and extracted with EtOAc (2×20 mL). The combined organic layers were washed with a 10% NH₄Cl aqueous solution (40 mL), dried over Na₂SO₄, filtered and concentrated. The residue was partitioned between EtOAc (20 mL) and HCl (3M, 20 mL). After stirring at 15° C. for 2 hours, the mixture was neutralized with a saturated aqueous solution of NaHCO₃ (60 mL). The organic layer was separated, washed with brine (20 mL), dried over Na₂SO₄, filtered and concentrated. The residue was purified by prep-HPLC (Column Phenomenex Gemini-NX C18 75*30 mm*3 um Condition A=water (0.225% FA)-B=ACN Begin B 48 End B 78 Gradient Time(min) 7 100% B Hold Time(min) 2 FlowRate(ml/min) 30 Injections 7) to give tert-butyl 4-fluoro-4-((6-(trifluoromethyl)pyridin-3-yl)methyl)piperidine-1-carboxylate (170 mg, 16.6%). ¹H-NMR (400 MHz, CDCl₃) δ_{H} 8.57 (s, 1H), 7.76 (d, J=8.0 Hz, 1H), 7.65 (d, J=8.1 Hz, 1H), 3.96 (s, 2H), 3.09-2.98 (m, 3H), 2.96 (s, 1H), 1.75-1.64 (m, 4H), 1.46 (s, 9H), 1.48-1.44 (m, 1H).

Step 7

To a solution of tert-butyl 4-fluoro-4-((6-(trifluoromethyl)pyridin-3-yl)methyl)piperidine-1-carboxylate (0.2 g, 0.5519 mmol) in dioxane (5 mL) was added HCl/dioxane (1.37 mL, 4M in dioxane, 5.51 mmol), the mixture was stirred at 25° C. for 4 hours. The mixture was concentrated to give 5-((4-fluoropiperidin-4-yl)methyl)-2-(trifluoromethyl)pyridine hydrochloride (200 mg, crude), which was used directly in the next reaction.

Step 8

To a mixture of 2-(pyrimidin-4-yl)nicotinic acid (121 mg, 0.6025 mmol), 5-((4-fluoropiperidin-4-yl)methyl)-2-(trifluoromethyl)pyridine hydrochloride (180 mg, 0.6025 mmol) and HATU (343 mg, 0.9037 mmol) in DMF (5 mL) was added DIPEA (0.31 mL, 1.80 mmol). The mixture was stirred at 20° C. for 12 hours. The mixture poured into H₂O (50 mL) and stirred for 20 minutes. The mixture was extracted with EtOAc (3×20 mL), and the combined organic layers were washed with brine (2×20 mL), dried over anhydrous Na₂SO₄, filtered and concentrated. The residue was purified by prep-HPLC (Column Phenomenex Gemini-NX 80*40 mm*3 um Condition A=water (0.05% NH₃H₂O)-B=ACN Begin B 26 End B 56 Gradient Time(min) 8 100% B Hold Time(min) 3.2 FlowRate(ml/min) 30 Injections 6) to give to afford (4-fluoro-4-((6-(trifluoromethyl)pyridin-3-yl)methyl)piperidin-1-yl)(2-(pyrimidin-4-yl)pyridin-3-yl)methanone (132 mg, 49.2%). ¹H-NMR (400 MHz, CDCl₃) δ_{H} 9.19 (s, 1H), 9.08 (s, 1H), 8.90 (m, 1H), 8.76 (m, 1H), 8.57 (s, 1H), 8.27 (d, J=4.8 Hz, 1H), 7.85-7.61 (m, 3H), 7.47 (m, 1H), 4.80-4.56 (m, 1H), 3.54-3.34 (m, 1H), 3.27-2.91 (m, 4H), 2.06-1.82 (m, 2H), 1.53 (s, 1H). ¹⁹F NMR (400 MHz, CDCl₃) δ_{F} −67.530, −163.259. LC-ELSD/MS purity ≥98%, MS ESI calcd. for C₂₂H₁₉F₄N₅O [M+H]⁺ 446.2, found 446.2.

### Example 26. Synthesis of (4-fluoro-4-((6-(trifluoromethyl)pyridin-2-yl)methyl)piperidin-1-yl)(2-(pyrimidin-4-yl)pyridin-3-yl)methanone (Cmpd 24)

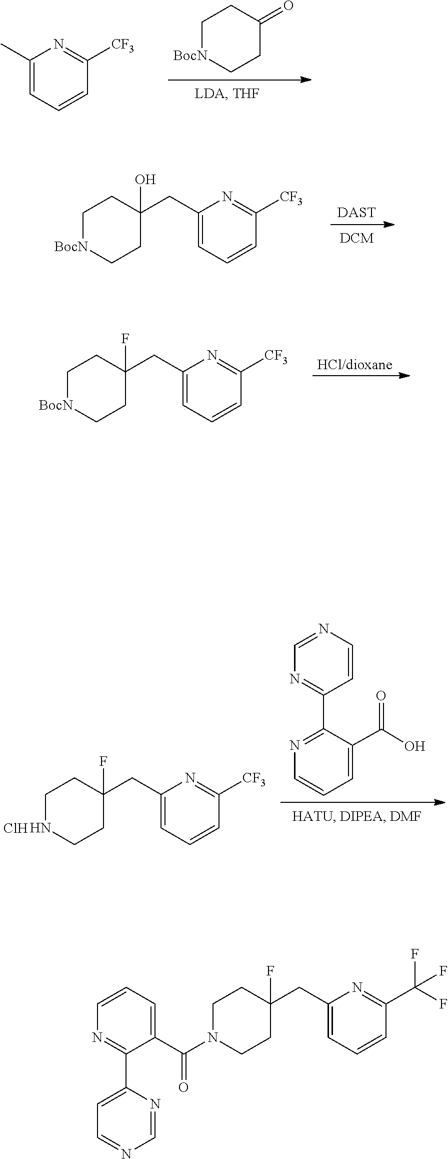

Step 1

To a solution of 2-methyl-6-(trifluoromethyl)pyri dine (2 g, 12.4 mmol) in THF (15 mL) was added dropwise LDA (9.30 mL, 2 M in THF, 18.6 mmol) at −78 TC. After stirring at −78° C. for 1 hour, a solution of tert-butyl 4-oxopiperidine-1-carboxylate (3.70 g, 18.6 mmol) in THF (10 mL) was added, and the reaction was stirred at −70° C. for 2 hours. The mixture was poured into water (100 mL) and extracted with EtOAc (3×50 mL). The combined organic layers were washed with brine (2×30 mL), dried over Na₂SO₄, filtered and concentrated. The residue was purified by prep-HPLC (Column: Xtimate C18 150*40 mm*5 um Condition A=water (0.04% NH₃H₂O+10 mM NH₄HCO₃)-B=ACN Begin B 38End B 68) to give tert-butyl 4-hydroxy-4-((6-(trifluoromethyl)pyridin-2-yl)methyl)piperidine-1-carboxylate (120 mg, impure), which was used directly in the next reaction.

Step 2

To a solution of tert-butyl 4-hydroxy-4-((6-(trifluoromethyl)pyridin-2-yl)methyl)piperidine-1-carboxylate (120 mg, 0.333 mmol) in DCM (10 mL) was added DAST (107 mg, 0.6658 mmol) at 0° C. The mixture was stirred at 0° C. for 5 minutes, then poured into an aqueous NaHCO₃ solution (30 mL) and stirred for 20 minutes. The mixture was extracted with DCM (3×20 mL), and the combined organic layers were washed with saturated brine (2×20 mL), dried over anhydrous Na₂SO₄, filtered and concentrated to give tert-butyl 4-fluoro-4-((6-(trifluoromethyl)pyridin-2-yl)methyl)piperidine-1-carboxylate (120 mg, crude), which was used directly in the next reaction.

Step 3

To a solution of tert-butyl 4-fluoro-4-((6-(trifluoromethyl)pyridin-2-yl)methyl)piperidine-1-carboxylate (120 mg, 0.33111 mmol) in dioxane (10 mL) was added HCl/dioxane (4.12 mL, 4M in dioxane, 1.65 mmol), and the mixture was stirred at 25° C. for 2 hours. The mixture was concentrated to give 2-((4-fluoropiperidin-4-yl)methyl)-6-(trifluoromethyl)pyridine hydrochloride (100 mg, crude), which was used directly in the next reaction.

Step 4

A mixture of 2-(pyrimidin-4-yl)nicotinic acid (80.8 mg, 0.4017 mmol), HATU (229 mg, 0.6025 mmol), DIPEA (258 mg, 2.00 mmol) and 2-((4-fluoropiperidin-4-yl)methyl)-6-(trifluoromethyl)pyridine hydrochloride (120 mg, 0.4017 mmol) in DMF (5 mL) was stirred at 25° C. for 2 hours. The mixture was poured into water (20 mL) and extracted with EtOAc (3×10 mL). The combined organic layers were washed with brine (2×20 mL), dried over anhydrous Na₂SO₄, filtered, and concentrated. The residue purified by prep-HPLC (Column Phenomenex Gemini-NX 80*40 mm*3 um Condition A=water (0.05% NH₃H₂O)-B=ACN Begin B 26 End B 56) to afford (50 mg, impure), which was further purified by SFC (Column DAICEL CHIRALCEL OD-H (250 mm*30 mm, 5 um) Condition A=0.1% NH₃H₂O B=EtOH Begin B 30% End B 30%) to give (4-fluoro-4-((6-(trifluoromethyl)pyridin-2-yl)methyl)piperidin-1-yl)(2-(pyrimidin-4-yl)pyridin-3-yl)methanone (35.6 mg, 20%). ¹H-NMR (400 MHz, CDCl₃) δ_{H} 9.11 (s, 1H), 8.79 (d, J=4.0 Hz, 1H), 8.68 (m, 1H), 8.17 (m, 1H), 7.76 (m, 1H), 7.48-7.66 (m, 2H), 7.34-7.44 (m, 2H), 4.37-4.66 (m, 1H), 3.25-3.41 (m, 1H), 3.03-3.24 (m, 4H), 1.68-2.02 (m, 3H), 1.13-1.29 (m, 1H). LC-ELSD/MS purity 99%; MS ESI calcd. for C₂₂H₂₀F₄N₅O [M+H]⁺ 446.3, found 446.3. ¹⁹F NMR (376.5 MHz, CDCl₃) δ_{F} −67.889; −157.22.

### Example 27. Synthesis of (4-fluoro-4-((4-(trifluoromethyl)pyridin-2-yl)methyl)piperidin-1-yl)(2-(pyrimidin-4-yl)pyridin-3-yl)methanone (Cmpd 25)

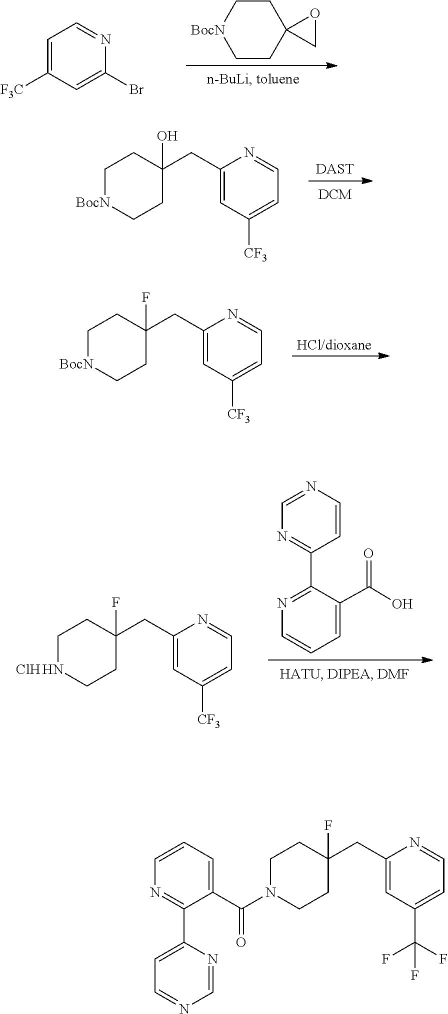

Step 1

To a solution of 2-bromo-4-(trifluoromethyl)pyridine (2 g, 8.84 mmol) in toluene (20 mL) was added slowly a solution of n-BuLi (7.04 mL, 2.5M in hexane, 17.6 mmol) at −78° C. under nitrogen. The mixture was stirred at −78° C. for 30 minutes, and then a solution of tert-butyl 1-oxa-6-azaspiro[2.5]octane-6-carboxylate (1.98 g, 9.33 mmol) in toluene (10 mL) and BF₃·EtO₂ (1.20 g, 8.49 mmol) was added dropwise at −70° C. After stirring at −70° C. for 2 hours, the mixture was poured slowly into a saturated aqueous NH₄Cl solution (200 mL), and the aqueous phase was extracted with EtOAc (3×100 mL). The combined organic layers were washed with brine (2×100 mL), dried over anhydrous Na₂SO₄, filtered and concentrated. The residue was purified by HPLC (Column: Phenomenex Gemini NX C18 150*40 mm*5 um Condition A=water (0.04% NH₃H₂O+10 mM NH₄HCO₃)-B=ACN Begin B 32End B 62) to give tert-butyl 4-hydroxy-4-((4-(trifluoromethyl)pyridin-2-yl)methyl)piperidine-1-carboxylate (100 mg, 3.27%). ¹H-NMR (400 MHz, CDCl₃) δ_{H} 8.71-8.70 (m, 1H), 7.30-7.49 (m, 2H), 3.81 (s, 2H), 3.22 (s, 2H), 3.00 (s, 2H), 2.96 (s, 1H), 2.88 (s, 1H), 2.80 (s, 1H), 1.49-1.54 (m, 4H), 1.45 (s, 10H).

Step 2

To a solution of tert-butyl 4-hydroxy-4-((4-(trifluoromethyl)pyridin-2-yl)methyl)piperidine-1-carboxylate (100 mg, 0.2774 mmol) in DCM (10 mL) was added DAST (89.4 mg, 0.5548 mmol) at 0° C. The mixture was stirred at 0° C. for 5 minutes and then poured into an aqueous NaHCO₃ solution (30 mL) and stirred for 20 minutes. The mixture was extracted with DCM (3×10 mL), and the combined organic layers were washed with brine (2×10 mL), dried over anhydrous Na₂SO₄, filtered and concentrated to give tert-butyl 4-fluoro-4-((4-(trifluoromethyl)pyridin-2-yl)methyl)piperidine-1-carboxylate (100 mg, crude), which was used directly in the next reaction.

Step 3

To a solution of tert-butyl 4-fluoro-4-((4-(trifluoromethyl)pyridin-2-yl)methyl)piperidine-1-carboxylate (100 mg, 0.2759 mmol) in dioxane (10 mL) was added HCl/dioxane (0.342 mL, 4M in dioxane, 1.37 mmol) and the mixture was stirred at 25° C. for 2 hours. The mixture was concentrated to give 2-((4-fluoropiperidin-4-yl)methyl)-4-(trifluoromethyl)pyridine hydrochloride (100 mg, crude), which was used directly in the next reaction.

Step 4

2-(Pyrimidin-4-yl)nicotinic acid (67.3 mg, 0.335 mmol), HATU (190 mg, 0.502 mmol), DIPEA (215 mg, 1.67 mmol) and 2-((4-fluoropiperidin-4-yl)methyl)-4-(trifluoromethyl)pyridine hydrochloride (100 mg, 0.335 mmol) were combined in DMF (5 mL) and stirred at 25° C. for 2 hours. The mixture was poured into water (20 mL) and extracted with EtOAc (3×15 mL). The combined organic layers were washed with brine (2×20 mL), dried over anhydrous Na₂SO₄, filtered and concentrated. The residue was purified by prep-HPLC (Column: Phenomenex Gemini-NX 80*40 mm*3 um Condition A=water (0.05% NH₃H₂O)-B=ACN Begin B 25End B 55) to afford (25 mg, impure), which was further purified by SFC (Column Phenomenex Gemini-NX 80*40 mm*3 um Condition A=water (0.05% NH₃H₂O+10 mM NH₄HCO₃)-B=ACN Begin B 40 End B 40 Gradient Time(min) 8 100% B Hold Time(min) 2.5FlowRate(ml/min) 30 Injections 4) to give (4-fluoro-4-((4-(trifluoromethyl)pyridin-2-yl)methyl)piperidin-1-yl)(2-(pyrimidin-4-yl)pyridin-3-yl)methanone (11 mg, 7%). ¹H-NMR (400 MHz, CDCl₃) δ_{H} 9.12-9.24 (m, 1H), 8.81-8.97 (m, 1H), 8.73 (s, 2H), 8.17-8.31 (m, 1H), 7.59-7.74 (m, 1H), 7.45 (s, 3H), 4.50-4.76 (m, 1H), 3.12-3.53 (m, 5H), 1.69-2.11 (m, 3H), 1.25 (s, 1H). LC-ELSD/MS purity 99%; MS ESI calcd. for C₂₂H₂₀F₄N₅O [M+H]⁺ 446.2, found 446.2. ¹⁹F-NMR (376.5 MHz, CDCl₃) δ_{F} −64.454; −159.925.

### Example 28. Synthesis of (4-fluoro-4-(2,4,6-trifluorobenzyl)piperidin-1-yl)(2-(pyrimidin-4-yl)pyridin-3-yl)methanone (Cmpd 26)

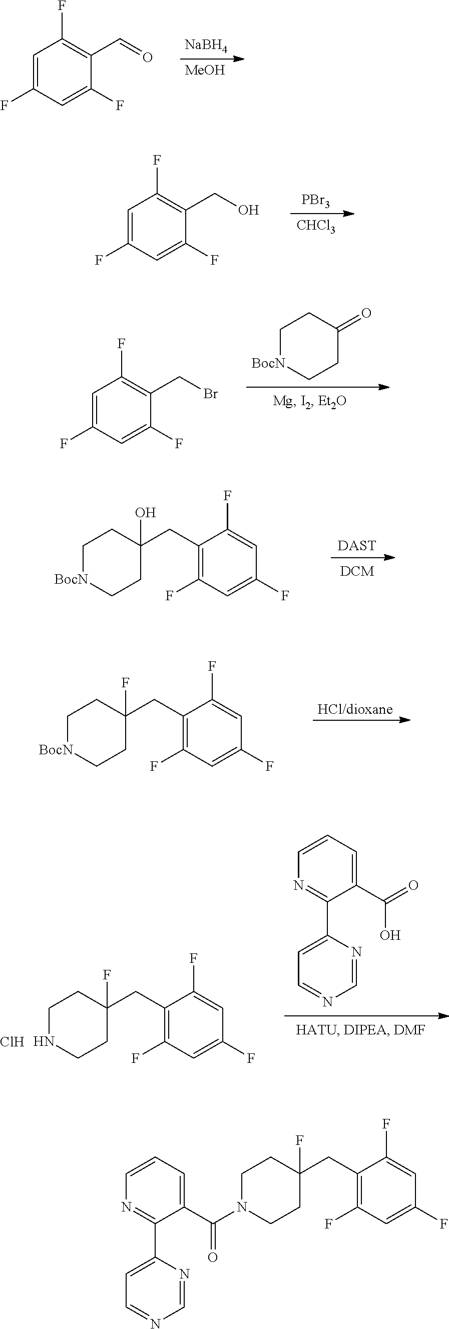

Step 1

To NaBH₄ (2.37 g, 62.4 mmol) in MeOH (50 mL) was added 2,4,6-trifluorobenzaldehyde (5 g, 31.2 mmol) and the resulting mixture was stirred at 25° C. for 1 hour. A saturated aqueous Na₂S₂O₃ solution (50 mL) was added and stirring was continued for 30 minutes. The mixture was extracted with EtOAc (2×50 mL), and the combined organic layers were dried over anhydrous Na₂SO₄, filtered and concentrated to give (2,4,6-trifluorophenyl)methanol (5 g, crude). ¹H-NMR (400 MHz, CDCl₃) S_{H} 6.76-6.57 (m, 2H), 4.81-4.65 (m, 2H), 1.89-1.80 (m, 1H). ¹⁹F NMR (376.5 MHz, CDCl₃) δ_{F} −107.514, −113.359.

Step 2.

To a solution of (2,4,6-trifluorophenyl)methanol (5.0 g, 30.8 mmol) in CHCl₃ (30 mL) was added tribromophosphane (16.6 g, 61.6 mmol) in one portion at 25° C., and the mixture was stirred at 25° C. for 0.5 hours. The mixture was slowly poured into ice-water (20 mL) and extracted with DCM (3×25 mL). The combined organic layers were washed with brine (2×20 mL), dried over anhydrous Na₂SO₄, filtered and concentrated to give 2-(bromomethyl)-1,3,5-trifluorobenzene (4.5 g, 64.9%). ¹H-NMR (400 MHz, CDCl₃) δ_{H} 6.76-6.59 (m, 2H), 4.48 (s, 2H). ¹⁹F NMR (376.5 MHz, CDCl₃) δ_{F} −105.975, −111.206.

Step 3

To a mixture of Mg (0.80 g, 33.3 mmol) and I₂ (2.81 mg, 0.011 mmol) in Et₂O (10 mL) was added 2-(bromomethyl)-1,3,5-trifluorobenzene (2.5 g, 11.1 mmol) in Et₂O (15 mL) at 25° C. under nitrogen. The mixture was stirred at 50° C. for 1 hour. The mixture was cooled, and tert-butyl 4-oxopiperidine-1-carboxylate (1.10 g, 5.55 mmol) was added the mixture at 25° C. The mixture was stirred at 25° C. for 2 hours and then was poured into ice-water (30 mL) and stirred for 20 minutes. The mixture was extracted with EtOAc (3×20 mL), and the combined organic layers were washed with brine (2×20 mL), dried over anhydrous Na₂SO₄, filtered and concentrated. The residue was purified by silica gel chromatography (0-30% of EtOAc in PE) to give tert-butyl 4-hydroxy-4-(2,4,6-trifluorobenzyl)piperidine-1-carboxylate (0.7 g, impure). ¹H-NMR (400 MHz, CDCl₃) δ_{H} 6.75-6.63 (m, 2H), 3.96-3.67 (m, 2H), 3.15-3.07 (m, 2H), 2.78 (s, 2H), 1.67-1.62 (m, 1H), 1.54-1.43 (m, 12H). ¹⁹F-NMR (376.5 MHz, CDCl₃) δ_{F} −109.293.

Step 4

To tert-butyl 4-hydroxy-4-(2,4,6-trifluorobenzyl)piperidine-1-carboxylate (0.7 g, 2.02 mmol) in DCM (10 mL) was added DAST (1.01 g, 6.06 mmol) at −10° C. The mixture was stirred at −10° C. for 15 minutes, and then poured slowly into ice-water (20 mL). The mixture was extracted with DCM (2×20 mL), and the combined organic layers were washed with NaHCO₃ (20 mL) and brine (10 mL), dried over anhydrous Na₂SO₄, concentrated to give tert-butyl 4-fluoro-4-(2,4,6-trifluorobenzyl)piperidine-1-carboxylate (600 mg, crude), which was used directly in the next reaction.

Step 5

To a solution of tert-butyl 4-fluoro-4-(2,4,6-trifluorobenzyl)piperidine-1-carboxylate (0.6 g, 1.72 mmol) in dioxane (10 mL) was added HCl/dioxane (4.3 mL, 4M HCl in dioxane) at 25° C. under nitrogen. The mixture was stirred at 25° C. for 16 hours. The reaction mixture was concentrated to give 4-fluoro-4-(2,4,6-trifluorobenzyl)piperidine hydrochloride (450 mg, crude), which was used directly in the next reaction.

Step 6

2-(Pyrimidin-4-yl)nicotinic acid (412 mg, 2.05 mmol), HATU (901 mg, 2.37 mmol), DIPEA (1.02 mg, 7.9 mmol) and 4-fluoro-4-(2,4,6-trifluorobenzyl)piperidine hydrochloride (450 mg, 1.58 mmol) were combined in DMF (5 mL) and stirred at 25° C. for 2 hours. The mixture was poured to water (10 mL) and extracted with EtOAc (3×10 mL). The combined organic layers were washed with brine (2×10 mL), dried over anhydrous Na₂SO₄, filtered and concentrated. The residue was purified by SFC (Column: Phenomenex-Cellulose-2 (250 mm*30 mm, 10 um); Condition: A=0.1% NH₃H₂O B=IPA; Begin B: 35; End B: 35) to afford (4-fluoro-4-(2,4,6-trifluorobenzyl)piperidin-1-yl)(2-(pyrimidin-4-yl)pyridin-3-yl)methanone (252 mg, 41.9%). ¹H-NMR (400 MHz, CDCl₃) δ_{H} 9.02-8.69 (m, 3H), 8.29-8.01 (m, 1H), 7.74-7.63 (m, 1H), 7.50-7.40 (m, 1H), 6.76-6.62 (m, 2H), 4.85-4.61 (m, 1H), 3.51-2.93 (m, 5H), 2.01-1.60 (m, 4H). LCMS purity 99%, calcd. for C₂₂H₂₀F₄N₄O [M+H]⁺ 431.2, found 431.2. ¹⁹F NMR (376.5 MHz, CDCl₃) δ_{F} −109.228, −161.132.

### Example 29. Synthesis of (4-fluoro-4-((5-(trifluoromethoxy)pyridin-2-yl)methyl)piperidin-1-yl)(2-(pyrimidin-4-yl)pyridin-3-yl)methanone (Cmpd27)

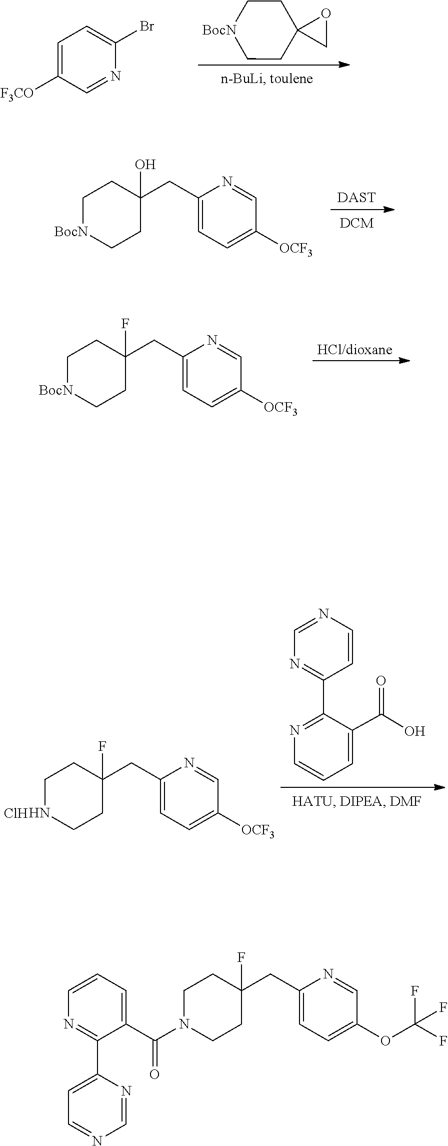

Step 1

To a solution of 2-bromo-5-(trifluoromethoxy)pyridine (2 g, 8.26 mmol) in toluene (20 mL) was added slowly a solution of n-BuLi (6.60 mL, 2.5M in hexane, 16.5 mmol) at −78° C. under nitrogen. The mixture was stirred at −78° C. for 30 minutes. A solution of tert-butyl 1-oxa-6-azaspiro[2.5]octane-6-carboxylate (1.80 g, 8.45 mmol) in toluene (10 mL) and BF₃·Et₂O (1.09 g, 7.69 mmol) was added dropwise at −70° C. under nitrogen. After stirring at −70° C. for 2 hours, the reaction mixture was poured slowly into a saturated aqueous NH₄Cl solution (100 mL). The mixture was extracted with EtOAc (3×30 mL) and the combined organic layers were washed with brine (2×40 mL), dried over anhydrous Na₂SO₄, filtered and concentrated. The residue was purified by prep-HPLC (Column: Phenomenex Gemini NX C18 150*40 mm*5 um Condition A=water (0.04% NH₃H₂O+10 mM NH₄HCO₃)-B=ACN Begin B 32End B 62) to give tert-butyl 4-hydroxy-4-((5-(trifluoromethoxy)pyridin-2-yl)methyl)piperidine-1-carboxylate (150 mg, impure). ¹H-NMR (400 MHz, CDCl₃) δ_{H} 8.46-8.45 (d, J=4.0 Hz, 1H), 7.49-7.56 (m, 1H), 7.20-7.18 (d, J=8.0 Hz, 1H), 5.18 (s, 1H), 3.69-3.87 (m, 2H), 3.13-3.29 (m, 2H), 2.93 (s, 2H), 1.47-1.52 (m, 4H), 1.45 (s, 10H).

Step 2

To tert-butyl 4-hydroxy-4-((5-(trifluoromethoxy)pyridin-2-yl)methyl)piperidine-1-carboxylate (150 mg, 0.3985 mmol) in DCM (10 mL) was added DAST (128 mg, 0.797 mmol) at 0° C. The mixture was stirred at 0° C. for 5 minutes. Then the mixture was poured into an aqueous NaHCO₃ solution (80 mL) and stirred for 20 minutes. The mixture was extracted with DCM (3×30 mL), and the combined organic layers were washed with brine (2×20 mL), dried over anhydrous Na₂SO₄, filtered and concentrated to give tert-butyl 4-fluoro-4-((5-(trifluoromethoxy)pyridin-2-yl)methyl)piperidine-1-carboxylate (150 mg, crude), which was used directly in the next reaction.

Step 3

To tert-butyl 4-fluoro-4-((5-(trifluoromethoxy)pyridin-2-yl)methyl)piperidine-1-carboxylate (150 mg, 0.3964 mmol) in dioxane (10 mL) was added HCl/dioxane (0.495 mL, 4M in dioxane, 1.98 mmol) and the mixture was stirred at 25° C. for 2 hours. The mixture was concentrated to give 2-((4-fluoropiperidin-4-yl)methyl)-5-(trifluoromethoxy)pyridine hydrochloride (150 mg, crude), which was used directly in the next reaction.

Step 4

2-(Pyrimidin-4-yl)nicotinic acid (95.8 mg, 0.4766 mmol), HATU (271 mg, 0.7149 mmol), DIPEA (307 mg, 2.38 mmol) and 2-((4-fluoropiperidin-4-yl)methyl)-5-(trifluoromethoxy)pyridine hydrochloride (150 mg, 0.4766 mmol) were combined in DMF (5 mL) and stirred at 25° C. for 2 hours. The mixture was poured into water (20 mL) and extracted with EtOAc (3×10 mL). The combined organic layers were washed with brine (2×20 mL), dried over anhydrous Na₂SO₄, filtered and concentrated. The residue was purified by silica gel chromatography (0˜3% of MeOH in DCM) to afford the desire product (300 mg, impure), which was further purified by prep-HPLC (Column. DAICEL CHIRALCEL OD-H (250 mm*30 mm, 5 um) Condition A=0.1% NH₃H₂O B=EtOH Begin B 25% End B 25%)) to give (4-fluoro-4-((5-(trifluoromethoxy)pyridin-2-yl)methyl)piperidin-1-yl)(2-(pyrimidin-4-yl)pyridin-3-yl)methanone (28.6 mg, 13%). ¹H-NMR (400 MHz, CDCl₃) δ_{H} 9.18-9.01 (s, 1H), 8.87 (d, J=4.0 Hz, 1H), 8.74 (m, 1H), 8.47 (s, 1H), 8.24 (m, 1H), 7.68-7.65 (m, 1H), 7.42-7.55 (m, 2H), 7.32 (m, 1H), 4.51-4.70 (m, 1H), 3.32-3.50 (m, 1H), 3.07-3.27 (m, 4H), 1.86-2.08 (m, 2H), 1.68 (s, 2H). LC-ELSD/MS purity 99%, MS ESI calcd. for C₂₂H₂₀F₄N₅O₂[M+H]⁺ 462.1, found 462.1. ¹⁹F-NMR (376.5 MHz, CDCl₃) δ_{F} −58.176; −160.589.

### Example 30. Synthesis of (4-fluoro-4-(2-fluoro-4-(trifluoromethyl)benzyl)piperidin-1-yl)(2-(pyrimidin-4-yl)pyridin-3-yl)methanone (Cmpd 28)

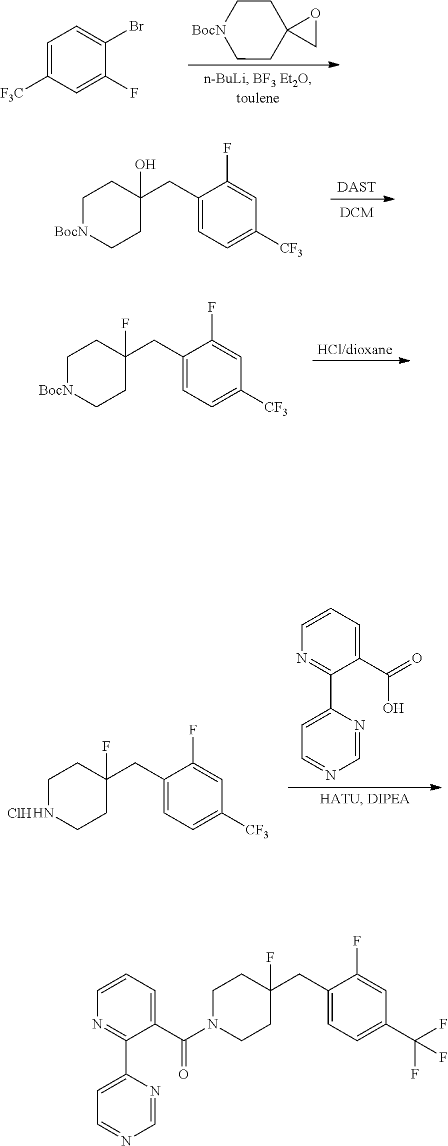

Step 1

To a solution of 1-bromo-2-fluoro-4-(trifluoromethyl)benzene (2 g, 8.23 mmol) in toluene (20 mL) was added slowly a solution of n-BuLi (6.55 mL, 2.5M in hexane, 16.4 mmol) at −78° C. under nitrogen, and the mixture was stirred at −78° C. for 30 minutes. To this mixture was added a solution of tert-butyl 1-oxa-6-azaspiro[2.5]octane-6-carboxylate (1.79 g, 8.40 mmol) in toluene (10 mL) and BF₃ Et₂O (1.08 g, 7.64 mmol) dropwise at −70° C. After stirring at −70° C. for 2 hours, the reaction mixture was poured slowly into a saturated aqueous NH₄Cl solution (100 mL). The mixture was extracted with EtOAc (3×40 mL), and the combined organic layers were washed with brine (2×20 mL), dried over anhydrous Na₂SO₄, filtered and concentrated. The residue was purified by prep-HPLC (Column: Phenomenex Gemini NX C18 150*40 mm*5 um Condition A=water (0.04% NH₃H₂O+10 mM NH₄HCO₃)-B=ACN Begin B 54End B 64) tert-butyl 4-fluoro-4-(2-fluoro-4-(trifluoromethyl)benzyl)piperidine-1-carboxylate (500 mg, impure). ¹H-NMR (400 MHz, CDCl₃) S_{H} 7.36-7.39 (m, 2H), 7.33 (d, J=8.0 Hz, 1H), 3.87 (s, 2H), 3.10 (m, 2H), 2.87 (s, 2H), 1.59-1.67 (m, 2H), 1.50 (s, 1H), 1.45 (s, 10H).

Step 2

To tert-butyl 4-fluoro-4-(2-fluoro-4-(trifluoromethyl)benzyl)piperidine-1-carboxylate (500 mg, 1.32 mmol) in DCM (10 mL) was added DAST (425 mg, 2.64 mmol) at 0° C. The mixture was stirred at 0° C. for 5 minutes and then was poured into an aqueous NaHCO₃ solution (100 mL) and stirred for 20 minutes. The mixture was extracted with DCM (3×40 mL), and the combined organic layers were washed with brine (2×20 mL), dried over anhydrous Na₂SO₄, filtered and concentrated to give tert-butyl 4-(2-fluoro-4-(trifluoromethyl)benzyl)-4-hydroxypiperidine-1-carboxylate (500 mg, crude), which was used directly in the next reaction.

Step 3

To tert-butyl 4-(2-fluoro-4-(trifluoromethyl)benzyl)-4-hydroxypiperidine-1-carboxylate (500 mg, 1.31 mmol) in dioxane (10 mL) was added HCl/dioxane (1.63 mL, 4M in dioxane, 6.55 mmol), and the mixture was stirred at 25° C. for 2 hours. The mixture was concentrated to give 4-fluoro-4-(2-fluoro-4-(trifluoromethyl)benzyl)piperidine hydrochloride (460 mg, crude), which was used directly in the next reaction.

Step 4

2-(Pyrimidin-4-yl)nicotinic acid (140 mg, 0.6967 mmol), HATU (361 mg, 0.9501 mmol), DIPEA (408 mg, 3.16 mmol) and 4-fluoro-4-(2-fluoro-4-(trifluoromethyl)benzyl)piperidine hydrochloride (200 mg, 0.6334 mmol) were combined in DMF (10 mL) and stirred at 25° C. for 2 hours. The mixture was poured into water (40 mL) and extracted with EtOAc (3×20 mL). The combined organic layers were washed with brine (2×20 mL), dried over anhydrous Na₂SO₄, filtered and concentrated. The residue was purified by silica gel chromatography (0˜3% of MeOH in DCM) to afford the desired product (330 mg, impure), which was further purified by prep-HPLC (Column: DAICEL CHIRALPAK AS (250 mm*30 mm, 10 um) Condition A=0.1% NH₃H₂O B=EtOH Begin B 15End B 15) to give (4-fluoro-4-(2-fluoro-4-(trifluoromethyl)benzyl)piperidin-1-yl)(2-(pyrimidin-4-yl)pyridin-3-yl)methanone (76.7 mg, 26%). ¹H-NMR (400 MHz, CDCl₃) δ_{H} 9.18-8.99 (s, 1H), 8.89 (s, 1H), 8.75-8.74 (m, 1H), 8.23 (d, J=4.0 Hz, 1H), 7.66 (s, 1H), 7.28-7.50 (m, 4H), 4.69 (d, J=12.0 Hz, 1H), 3.33-3.49 (m, 1H), 2.97-3.27 (m, 4H), 1.59-2.10 (m, 4H); ¹⁹F-NMR (376.5 MHz, CDCl₃) S_{F}-62.708; −114.065; −161.752. LC-ELSD/MS purity 99%; MS ESI calcd. for C₂₃H₂₀F₅N₄O [M+H]⁺ 463.2, found 463.2.

### Example 31. Synthesis of (4-(4-chlorobenzyl)-4-fluoropiperidin-1-yl)(2-(pyrimidin-4-yl)pyridin-3-yl)methanone (Cmpd 29)

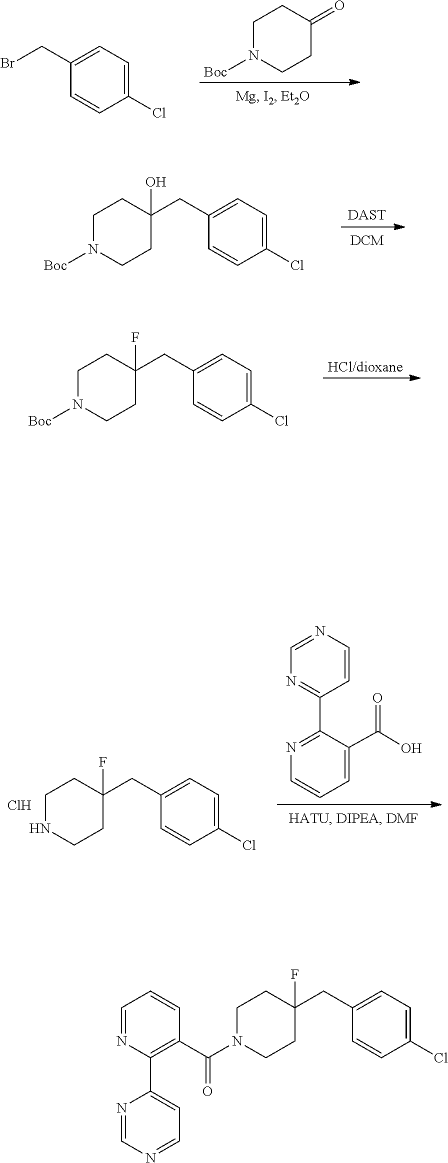

Step 1

To a mixture of Mg (0.35 g, 14.5 mmol) and I₂ (2.46 mg, 0.02 mmol) in Et₂O (5 mL) was added 1-(bromomethyl)-4-chlorobenzene (1 g, 4.86 mmol) in Et₂O (10 mL) at 25° C. under nitrogen. The mixture was stirred at 35° C. for 1 hour. tert-Butyl 4-oxopiperidine-1-carboxylate (0.773 g, 3.88 mmol) in Et₂O (10 mL) was added the mixture at 25° C. The mixture was stirred at 25° C. for 2 hours, and then the mixture was poured into ice-water (30 mL) and stirred for 20 minutes. The mixture was extracted with EtOAc (3×20 mL), and the combined organic phases were washed with brine (2×20 mL), dried over anhydrous Na₂SO₄, filtered and concentrated. The residue was purified by silica gel chromatography (0˜30% of EtOAc in PE) to give tert-butyl 4-(4-chlorobenzyl)-4-hydroxypiperidine-1-carboxylate (280 mg, 17.7%). ¹H-NMR (400 MHz, CDCl₃) δ_{H} 7.31-7.27 (m, 2H), 7.15-7.10 (m, 2H), 3.95-3.77 (m, 2H), 3.15-3.00 (m, 2H), 2.76-2.68 (m, 2H), 1.59-1.44 (m, 13H).

Step 2

To tert-butyl 4-(4-chlorobenzyl)-4-hydroxypiperidine-1-carboxylate (0.28 g, 0.86 mmol) in DCM (10 mL) was added DAST (0.429 g, 2.57 mmol) at −10° C. The mixture was stirred at −10° C. for 15 minutes and then poured slowly into ice-water (20 mL). The mixture was extracted with DCM (2×20 mL), and the combined organic layers were washed with NaHCO₃ (20 mL) and brine (10 mL), dried over anhydrous Na₂SO₄, filtered and concentrated to give tert-butyl 4-(4-chlorobenzyl)-4-fluoropiperidine-1-carboxylate (280 mg, crude), which was used directly in the next reaction.

Step 3

To a solution of tert-butyl 4-(4-chlorobenzyl)-4-fluoropiperidine-1-carboxylate (0.28 g, 0.85 mmol) in dioxane (5 mL) was added HCl/dioxane (2.13 mL, 4M HCl in dioxane) at 25° C. under nitrogen. The mixture was stirred at 25° C. for 2 hours and concentrated to give 4-(4-chlorobenzyl)-4-fluoropiperidine hydrochloride (200 mg, crude), which was used directly in the next reaction.

Step 4

2-(Pyrimidin-4-yl)nicotinic acid (201 mg, 1.0 mmol), HATU (0.47 g, 1.24 mmol), DIPEA (0.537 g, 4.16 mmol) and 4-(4-chlorobenzyl)-4-fluoropiperidine hydrochloride (220 mg, 0.83 mmol) were combined in DMF (5 mL) and stirred at 25° C. for 2 hours. The mixture was poured to water (10 mL) and extracted with EtOAc (3×10 mL). The combined organic layers were washed with brine (2×10 mL), dried over Na₂SO₄, filtered and concentrated. The residue was purified by silica gel chromatography (0˜20% of MeOH in DCM) to give the desired product (200 mg, impure). LC-ELSD/MS purity 83%, MS ESI calcd. for C₂₂H₂₁ClFN₄O [M+H]⁺ 411.1, found 411.1.

The residue was further purified by prep-SFC (Column: DAICEL CHIRALCEL OD-H (250 mm*30 mm, Sum); Condition: A=0.1% NH₃H₂O B=IPA; Begin B: 35; End B: 35) to afford a residue that was triturated from water (10 mL) at 80° C. to give (4-(4-chlorobenzyl)-4-fluoropiperidin-1-yl)(2-(pyrimidin-4-yl)pyridin-3-yl)methanone (23.7 mg, 7%). ¹H-NMR (400 MHz, CDCl₃) δ_{H} 9.25-8.72 (m, 3H), 8.28-8.19 (m, 1H), 7.71-7.69 (m, 1H), 7.47-7.41 (m, 1H), 7.32-7.27 (m, 2H), 7.16-7.08 (m, 2H), 4.75-4.52 (m, 1H), 3.44-3.37 (m, 1H), 3.25-3.06 (m, 2H), 2.99-2.85 (m, 2H), 1.99-1.61 (m, 3H), 1.55-1.48 (m, 1H). ¹⁹F-NMR (376.5 MHz, CDCl₃) δ_{F} −161.33. LC-ELSD/MS purity 99%, MS ESI calcd. for C₂₂H₂₁ClFN₄O [M+H]⁺ 411.1, found 411.2.

### Example 32. Synthesis of (4-((5-chloro-3-fluoropyridin-2-yl)methyl)-4-fluoropiperidin-1-yl)(2-(pyrimidin-4-yl)pyridin-3-yl)methanone (Cmpd 30)

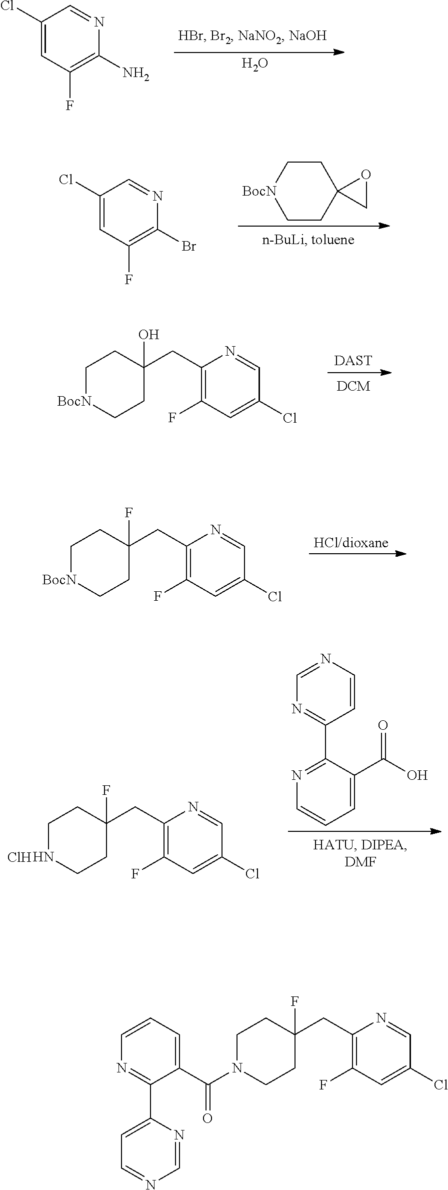

Step 1

To 5-chloro-3-fluoropyridin-2-amine (5.0 g, 34 mmol) was slowly added HBr (48%, 20 mL) with stirring at 0° C. The reaction mixture was cooled to −10° C. and a solution of NaNO₂ (5.88 g, 85.3 mmol) in water (20 mL) was added over 1.5 hours. The mixture was stirred for an additional 30 minutes at −10° C. Then a solution of NaOH (12 g, 300 mmol) in water (20 mL) was added over 30 minutes, and the mixture was allowed to warm to 20° C. The mixture was extracted with EtOAc (3×100 mL), and the combined organic layers were washed with brine (100 mL), dried over Na₂SO₄, filtered and concentrated to afford the crude product (6.0 g). A sample of the crude product (3 g, 14.1 mmol) was purified by silica gel chromatography (0˜30% of EtOAc in PE) to give (4-((5-chloro-3-fluoropyridin-2-yl)methyl)-4-fluoropiperidin-1-yl)(2-(pyrimidin-4-yl)pyridin-3-yl)methanone (2.3 g, 76.9%). ¹H-NMR (400 MHz, CDCl₃) δ_{H} 8.22 (d, J=2.0 Hz, 1H), 7.47 (d, J=8, 2.0 Hz, 1H), 1.99-2.08 (m, 1H), 1.60 (s, 1H).

Step 2

To a solution of (4-((5-chloro-3-fluoropyridin-2-yl)methyl)-4-fluoropiperidin-1-yl)(2-(pyrimidin-4-yl)pyridin-3-yl)methanone (2.3 g, 10.9 mmol) in toluene (20 mL) was added slowly a solution of n-BuLi (8.72 mL, 2.5M in hexane, 21.8 mmol) at −78° C. under nitrogen. After addition was complete, the mixture was stirred at −78° C. for 30 minutes. A solution of tert-butyl 1-oxa-6-azaspiro[2.5]octane-6-carboxylate (2.15 g, 10.1 mmol) in toluene (10 mL) and BF₃·Et₂O (1.43 g, 10.1 mmol) was added dropwise at −70° C. After stirring at −70° C. for 2 hours, the reaction mixture was poured slowly into a saturated aqueous NH₄Cl solution (200 mL). The mixture was extracted with EtOAc (3×80 mL) and the combined organic layers were washed with brine (2×50 mL), dried over anhydrous Na₂SO₄, filtered and concentrated. The residue was purified by silica gel chromatography (0˜20% of EtOAc in PE) to give tert-butyl 4-((5-chloro-3-fluoropyridin-2-yl)methyl)-4-hydroxypiperidine-1-carboxylate (800 mg, impure). ¹H-NMR (400 MHz, CDCl₃) δ_{H} 8.27-8.26 (d, J=4.0 Hz, 1H), 7.40 (m, 1H), 3.66-3.83 (m, 2H), 3.02-3.23 (m, 2H), 2.88-2.87 (d, J=4.0 Hz, 2H), 1.54 (s, 2H), 1.44-1.50 (m, 3H), 1.37-1.40 (m, 9H).

Step 3

To tert-butyl 4-((5-chloro-3-fluoropyridin-2-yl)methyl)-4-hydroxypiperidine-1-carboxylate (400 mg, 1.16 mmol) in DCM (10 mL) was added DAST (373 mg, 2.32 mmol) at 0° C. The mixture was stirred at 0° C. for 5 minutes and then poured slowly into an aqueous NaHCO₃ solution (100 mL) and stirred for 20 minutes. The mixture was extracted with DCM (3×40 mL), and the combined organic layers were washed with brine (2×30 mL), dried over anhydrous Na₂SO₄, filtered and concentrated to give tert-butyl 4-((5-chloro-3-fluoropyridin-2-yl)methyl)-4-fluoropiperidine-1-carboxylate (400 mg, crude), which was used directly in the next step.

Step 4

To tert-butyl 4-((5-chloro-3-fluoropyridin-2-yl)methyl)-4-fluoropiperidine-1-carboxylate (400 mg, 1.15 mmol) in dioxane (10 mL) was added HCl/dioxane (1.43 mL, 4M in dioxane, 5.75 mmol), and the mixture was stirred at 25° C. for 2 hours. The mixture was concentrated to give 5-chloro-3-fluoro-2-((4-fluoropiperidin-4-yl)methyl)pyridine hydrochloride (400 mg, crude), which was used directly in the next step.

Step 5

2-(Pyrimidin-4-yl)nicotinic acid (283 mg, 1.41 mmol), HATU (802 mg, 2.11 mmol), DIPEA (911 mg, 7.05 mmol) and 5-chloro-3-fluoro-2-((4-fluoropiperidin-4-yl)methyl)pyridine hydrochloride (400 mg, 1.5 mmol) were combined in DMF (5 mL) and stirred at 25° C. for 2 hours. The mixture was poured into water (30 mL) and extracted with EtOAc (3×20 mL). The combined organic layers were washed with brine (2×20 mL), dried over anhydrous Na₂SO₄, filtered, concentrated and purified by silica gel chromatography (0˜3% of MeOH in DCM), and further purified by SFC (Column: Phenomenex-Cellulose-2 (250 mm*30 mm 10 um) Condition A=0.1% NH₃H₂O B=EtOH Begin B 40End B 40) to give (4-((5-chloro-3-fluoropyridin-2-yl)methyl)-4-fluoropiperidin-1-yl)(2-(pyrimidin-4-yl)pyridin-3-yl)methanone (24.2 mg, 4%). ¹H-NMR (400 MHz, CDCl₃) δ_{H} 9.17 (s, 1H), 8.87 (s, 1H), 8.74 (m, 1H), 8.38 (s, 1H), 8.24-8.22 (d, J=8.0 Hz, 1H), 7.66 (d, J=8.0 Hz, 1H), 7.45 (d, J=8.0 Hz, 2H), 4.52-4.74 (m, 1H), 3.43 (s, 1H), 3.00-3.33 (m, 4H), 1.72-2.11 (m, 4H), 0.81-0.91 (m, 1H). ¹⁹F-NMR (376.5 MHz, CDCl₃) δ_{F} −120.045; −159.763. LC-ELSD/MS purity 99%; MS ESI calcd. for C₂₁H₁₉ClF₂N₅O [M+H]⁺ 430.1, found 430.1.

### Example 33. Synthesis of (R)-(4-(1-(3,5-difluorophenyl)ethyl)-4-fluoropiperidin-1-yl)(2-(pyrimidin-4-yl)pyridin-3-yl)methanone (Cmpd 31)

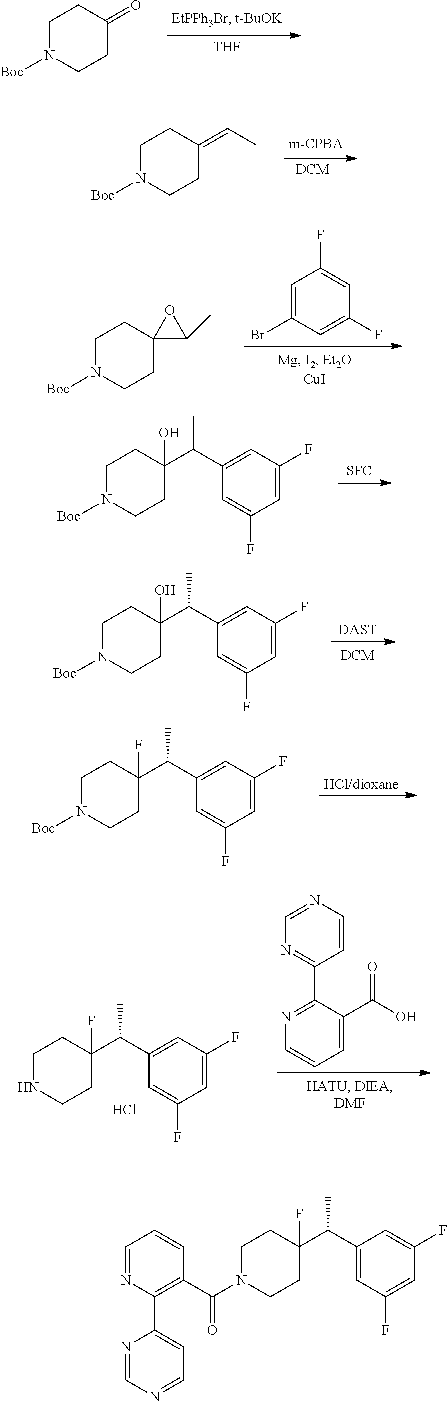

Step 1

To a mixture of EtPPh₃Br (74.2 g, 200 mmol) in THF (100 mL) was added t-BuOK (22.4 g, 200 mmol) at 25° C. under nitrogen. The resulting mixture was stirred at 60° C. for 30 minutes. tert-butyl 4-oxopiperidine-1-carboxylate (20 g, 100 mmol) in THE (100 mL) was added in portions so that the reaction temperature was maintained below 60° C. The reaction mixture was stirred at 60° C. for 16 h. The mixture was cooled and concentrated. The residue was poured into ice-water (150 mL), stirred for 20 minutes, and then was extracted with EtOAc (3×100 mL). The combined organic layers were washed with brine (2×100 mL), dried over anhydrous Na₂SO₄, filtered and concentrated. The residue was purified by silica gel chromatography (0˜100% of EtOAc in PE) to give tert-butyl 4-ethylidenepiperidine-1-carboxylate (20 g, impure). ¹H-NMR (400 MHz, CDCl₃) δ_{H} 5.32-5.23 (m, 1H), 3.42-3.32 (m, 4H), 2.23-2.09 (m, 4H), 1.60 (s, 3H), 1.47 (s, 9H).

Step 2

To a solution of tert-butyl 4-ethylidenepiperidine-1-carboxylate (15 g, 70.9 mmol) in DCM (50 mL) at 25° C. was added m-CPBA (15.3 g, 212 mmol). After stirring at 25° C. for 2 hours, the reaction mixture was quenched with a saturated aqueous NaHCO₃ solution (50 mL) and then saturated aqueous Na₂S₂O₃ (50 mL) was added. The mixture was extracted with DCM (2×50 mL), and the combined organic layers were washed with an aqueous NaHCO₃ solution (50 mL) and saturated aqueous Na₂S₂O₃ (50 mL), dried over Na₂SO₄, filtered and concentrated. The residue was purified by silica gel chromatography (0˜30% of EtOAc in PE) to give tert-butyl 2-methyl-1-oxa-6-azaspiro[2.5]octane-6-carboxylate (15 g, 93.1%). ¹H-NMR (400 MHz, CDCl₃) δ_{H} 3.79-3.64 (m, 2H), 3.44-3.32 (m, 2H), 2.96-2.89 (m, 1H), 1.47 (s, 9H), 1.44-1.34 (m, 2H).

Step 3

To a solution tert-butyl 2-methyl-1-oxa-6-azaspiro [2.5] octane-6-carboxylate (2.0 g, 8.79 mmol) in Et₂O (20 mL) was added CuI (834 mg, 4.39 mmol), bromo(3, 5-difluorophenyl) and magnesium (3.80 g, 17.5 mmol) at 25° C. The mixture was stirred at 25° C. for 2 hours. The mixture was poured into ice-water (30 mL) and stirred for 20 minutes. The mixture was extracted with EtOAc (3×20 mL), and the combined organic layers were washed with brine (2×20 mL), dried over anhydrous Na₂SO₄, filtered and concentrated. The residue was purified by silica gel chromatography (0˜30% of EtOAc in PE) to give tert-butyl 4-(1-(3,5-difluorophenyl)ethyl)-4-hydroxypiperidine-1-carboxylate (2 g, impure). ¹H-NMR (400 MHz, CDCl₃) δ_{H} 6.82-6.65 (m, 1H), 4.05-3.65 (m, 2H), 3.44-3.30 (m, 1H), 3.11-2.87 (m, 2H), 1.85-1.62 (m, 3H), 1.56-1.41 (m, 9H), 1.32-1.28 (m, 2H). ¹⁹F-NMR (376.5 MHz, CDCl₃) δ_{F} −110.06. A sample of the impure racemic product was further purified by prep-SFC (Column: DAICEL CHIRALPAK AD 250 mm*50 mm, 10 um); Condition: A=0.1% NH₃H₂O B=EtOH; Begin B: 45; End B: 45) to afford tert-butyl (R)-4-(1-(3,5-difluorophenyl)ethyl)-4-hydroxypiperidine-1-carboxylate (350 mg, 17.5%). ¹H-NMR (400 MHz, CDCl₃) δ_{H} 6.81-6.65 (m, 4H), 4.05-3.76 (m, 2H), 3.13-2.69 (m, 2H), 1.70-1.59 (m, 2H), 1.53-1.49 (m, 2H), 1.45 (m, 9H), 1.34-1.27 (m, 3H), 1.24-1.18 (m, 1H). ee=100%. ¹⁹F-NMR (376.5 MHz, CDCl₃) δ_{F} −110.04.

Step 4

To a mixture of tert-butyl (R)-4-(1-(3,5-difluorophenyl)ethyl)-4-hydroxypiperidine-1-carboxylate (300 mg, 0.74 mmol) in DCM (10 mL) was added DAST (245 mg, 1.47 mmol) at −10° C. The mixture was stirred at −10° C. for 15 minutes and then slowly added to ice-water (20 mL). The mixture was extracted with DCM (2×20 mL), and the combined organic layers were washed with NaHCO₃ (20 mL) and brine (10 mL), dried over anhydrous Na₂SO₄, filtered and concentrated to give tert-butyl (R)-4-(1-(3,5-difluorophenyl)ethyl)-4-fluoropiperidine-1-carboxylate (320 mg, crude). LC-ELSD/MS purity 70%, MS ESI calcd. for C₁₃H₁₇F₃N [M+H]⁺ 244.1, found 244.1.

Step 5

To a solution of tert-butyl (R)-4-(1-(3,5-difluorophenyl)ethyl)-4-fluoropiperidine-1-carboxylate (320 mg, 0.93 mmol) in dioxane (5 mL) was added HCl/dioxane (2.32 mL, 4 M HCl in dioxane) at 25° C. under nitrogen. The mixture was stirred at 25° C. for 2 hours. The reaction mixture was concentrated to give (R)-4-(1-(3,5-difluorophenyl)ethyl)-4-fluoropiperidine hydrochloride (220 mg, crude). LC-ELSD/MS purity 90%, MS ESI calcd. for C₁₃H₁₇F₃N [M+H]⁺ 244.1, found 244.1.

Step 6

2-(Pyrimidin-4-yl)nicotinic acid (217 mg, 1.08 mmol), HATU (0.513 g, 1.35 mmol), DIPEA (0.584 g, 4.52 mmol) and (R)-4-(1-(3,5-difluorophenyl)ethyl)-4-fluoropiperidine hydrochloride (220 mg, 0.904 mmol) were combined in DMF (5 mL) and stirred at 25° C. for 2 hours. The mixture was poured into water (20 mL) and extracted with EtOAc (3×10 mL). The combined organic layers were washed with brine (2×20 mL), dried over anhydrous Na₂SO₄, filtered and concentrated. The residue was purified by silica gel chromatography (0˜10% of MeOH in DCM) to afford (300 mg, impure), which was further purified by HPLC (Column: Phenomenex Gemini-NX C18 75*30 mm*3 um; Condition: A=water (0.225% FA)-B=ACN; Begin B: 45%; End B:65%) to afford (50 mg, impure), which was purified by SFC (Column: DAICEL CHIRALCEL OD-H (250 mm*30 mm, Sum); Condition: A=0.1% NH₃H₂O B=EtOH; Begin B: 25%; End B: 25%) to afford (R)-(4-(1-(3,5-difluorophenyl)ethyl)-4-fluoropiperidin-1-yl)(2-(pyrimidin-4-yl)pyridin-3-yl)methanone (10.9 mg, 3%). ¹H-NMR (400 MHz, CDCl₃)^{6H}8.93-8.66 (m, 3H), 8.28-8.19 (m, 1H), 7.75-7.61 (m, 1H), 7.50-7.40 (m, 1H), 6.80-6.64 (m, 3H), 4.79-4.57 (m, 1H), 3.50-2.73 (m, 5H), 2.27-1.65 (m, 2H), 1.41-1.29 (m, 4H). ¹⁹F-NMR (376.5 MHz, CDCl₃) δ_{F} −109.81, −170.14, −171.74. LC-ELSD/MS purity 99%, MS ESI calcd. for C₂₃H₂₁F₃N₄ONa [M+Na]⁺ 449.2, found 449.2.

### Example 34. Synthesis of (S)-(4-(1-(3,5-difluorophenyl)ethyl)-4-fluoropiperidin-1-yl)(2-(pyrimidin-4-yl)pyridin-3-yl)methanone (Cmpd 32)

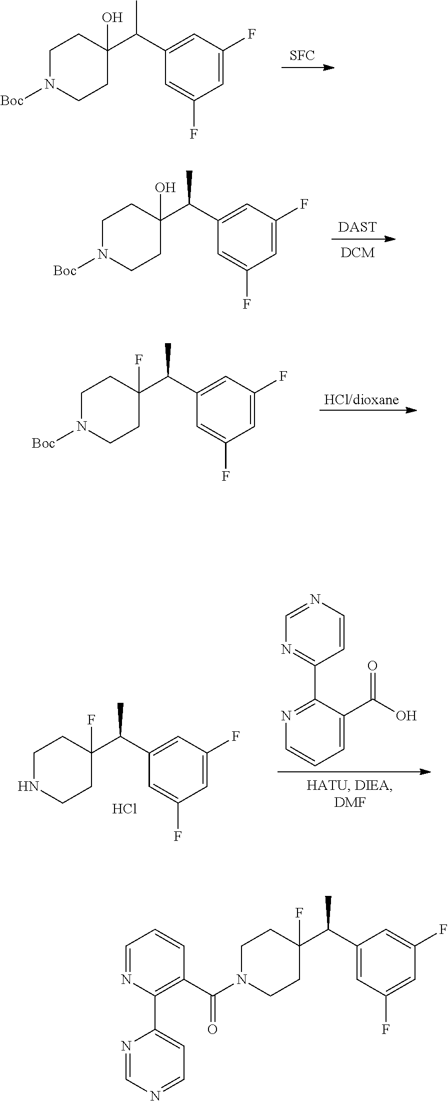

Step 1

Racemic-tert-butyl 4-(1-(3,5-difluorophenyl)ethyl)-4-hydroxypiperidine-1-carboxylate (1.00 g, impure) was purified by SFC (Column: DAICEL CHIRALPAK AD 250 mm*50 mm, 10 um); Condition: A=0.1% NH₃H₂O B=EtOH; Begin B: 45; End B: 45) to afford the desired product (1 g, impure). The residue was further purified by SFC (Column: DAICEL CHIRALPAK AD (250 mm*50 mm, 10 um); Condition: A=0.1% NH₃H₂O B=EtOH; Begin B: 15%; End B:15%) to afford tert-butyl (S)-4-(1-(3,5-difluorophenyl)ethyl)-4-hydroxypiperidine-1-carboxylate (350 mg, 35.1%). ¹H-NMR (400 MHz, CDCl₃) δ_{H} 6.81-6.65 (m, 4H), 4.03-3.72 (m, 2H), 3.17-2.88 (m, 2H), 2.73-2.34 (m, 1H), 1.72-1.57 (m, 1H), 1.55-1.47 (m, 3H), 1.45 (m, 9H), 1.33-1.18 (m, 4H). ¹⁹F-NMR (376.5 MHz, CDCl₃) δ_{F} −110.04.

Step 2

To tert-butyl (S)-4-(1-(3,5-difluorophenyl)ethyl)-4-hydroxypiperidine-1-carboxylate (0.35 g, 1.02 mmol) in DCM (10 mL) was added DAST (425 mg, 2.55 mmol) at −10° C. After stirring at −10° C. for 15 minutes, the mixture was slow poured into ice-water (20 mL). The mixture was extracted with DCM (2×20 mL) and the combined organic layers were washed with NaHCO₃ (20 mL) and brine (10 mL), dried over anhydrous Na₂SO₄, filtered and concentrated to give tert-butyl (S)-4-(1-(3,5-difluorophenyl)ethyl)-4-fluoropiperidine-1-carboxylate (320 mg, crude), which was used directly in the next reaction.

Step 3

To a solution of tert-butyl (S)-4-(1-(3,5-difluorophenyl)ethyl)-4-fluoropiperidine-1-carboxylate (0.32 g, 0.93 mmol) in dioxane (5 mL) was added HCl/dioxane (2.32 mL, 4M HCl in dioxane) at 25° C. under nitrogen. The mixture was stirred at 25° C. for 2 hours. The reaction mixture was concentrated to give (S)-4-(1-(3,5-difluorophenyl)ethyl)-4-fluoropiperidine hydrochloride (220 mg, crude), which was used directly in the next reaction.

Step 4

2-(Pyrimidin-4-yl)nicotinic acid (217 mg, 1.08 mmol), HATU (0.513 g, 1.35 mmol), DIPEA (0.584 g, 4.52 mmol) and (S)-4-(1-(3,5-difluorophenyl)ethyl)-4-fluoropiperidine hydrochloride (220 mg, 0.904 mmol) were combined in DMF (5 mL) and stirred at 25° C. for 2 hours. The mixture was poured into water (20 mL) and extracted with EtOAc (3×10 mL). The combined organic layers were washed with brine (2×20 mL), dried over anhydrous Na₂SO₄, filtered and concentrated. The residue was purified by HPLC (Column: Phenomenex Gemini-NX C18 75*30 mm*3 um; Condition: A=water (0.225% FA)-B=ACN; Begin B: 40%; End B:70%) to afford (90 mg, impure). The impure residue further was purified by SFC (Column: DAICEL CHIRALPAK AD (250 mm*30 mm, 10 um); Condition: A=0.1% NH₃H₂O B=EtOH; Begin B: 30%; End B:30%) to afford (S)-(4-(1-(3,5-difluorophenyl)ethyl)-4-fluoropiperidin-1-yl)(2-(pyrimidin-4-yl)pyridin-3-yl)methanone (40.6 mg, 11%). ¹H-NMR (400 MHz, CDCl₃) δ_{H} 8.95-8.69 (m, 3H), 8.34-8.16 (m, 1H), 7.72-7.62 (m, 1H), 7.53-7.36 (m, 1H), 6.84-6.65 (m, 3H), 4.84-4.51 (m, 1H), 3.53-2.73 (m, 5H), 2.27-1.65 (m, 2H), 1.47-1.29 (m, 4H). ¹⁹F-NMR (376.5 MHz, CDCl₃) δ_{F} −109.83, −170.14, −171.74. LC-ELSD/MS purity 99%, MS ESI calcd. for C₂₃H₂₂F₃N₄O [M+H]⁺ 427.3, found 427.3.

### Example 35 and Example 36. Synthesis of (S)-(4-fluoro-4-(1-(4-(trifluoromethyl)phenyl)ethyl)piperidin-1-yl)(2-(pyrimidin-4-yl)pyridin-3-yl)methanone (Cmpd 33) and Synthesis of (R)-(4-fluoro-4-(1-(4-(trifluoromethyl)phenyl)ethyl)piperidin-1-yl)(2-(pyrimidin-4-yl)pyridin-3-yl)methanone (Cmpd 34)

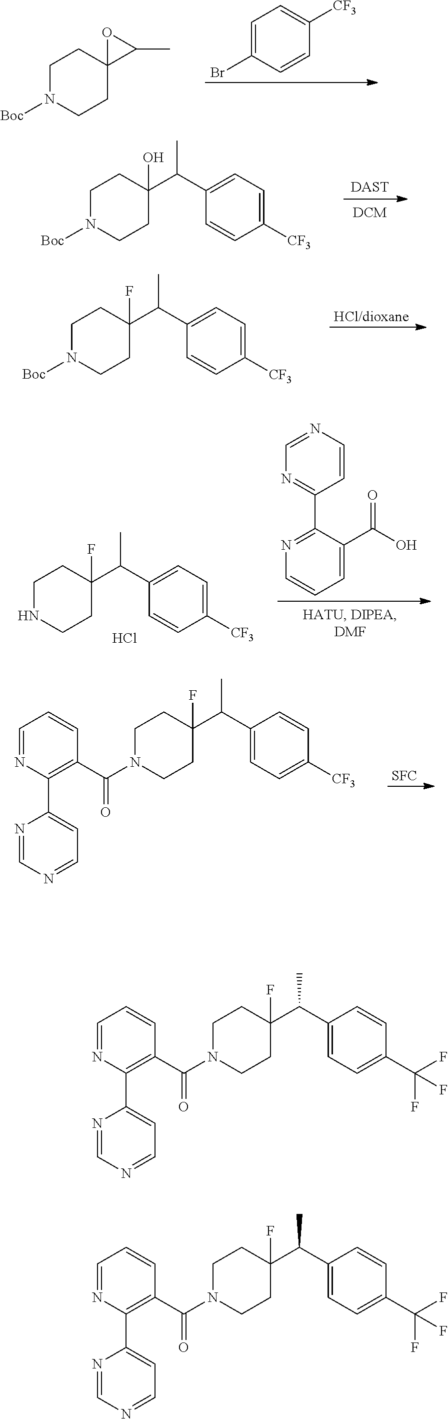

Step 1

To a mixture of Mg (1.26 g, 52.7 mmol) and I₂ (2.23 mg, 0.01 mmol) in Et₂O (10 mL) was added 1-bromo-4-(trifluoromethyl)benzene (3.93 g, 17.5 mmol) in Et₂O (20 mL) at 25° C. under nitrogen. The mixture was stirred at 35° C. for 1 hour, and then tert-butyl 2-methyl-1-oxa-6-azaspiro[2.5]octane-6-carboxylate (2.0 g, 8.79 mmol) in Et₂O (20 mL) was added to the mixture at 25° C. The mixture was stirred at 25° C. for 2 hours. The mixture was poured into ice-water (30 mL), stirred for 20 minutes and then extracted with EtOAc (3×20 mL). The combined organic layers were washed with brine (2×20 mL), dried over anhydrous Na₂SO₄, filtered and concentrated. The residue was purified by silica gel chromatography (0˜30% of EtOAc in PE) to give tert-butyl 4-hydroxy-4-(1-(4-(trifluoromethyl)phenyl)ethyl)piperidine-1-carboxylate (2 g, 60.9%). ¹H-NMR (400 MHz, CDCl₃) δ_{H} 7.61-7.53 (m, 2H), 7.39-7.30 (m, 2H), 4.04-3.77 (m, 2H), 3.11-2.89 (m, 2H), 2.83-2.73 (m, 1H), 1.60-1.48 (m, 3H), 1.46-1.43 (m, 10H), 1.36-1.31 (m, 3H), 1.22-1.14 (m, 1H). ¹⁹F-NMR (376.5 MHz, CDCl₃) δ_{F} −62.45.

Step 2

To a mixture of tert-butyl 4-hydroxy-4-(1-(4-(trifluoromethyl)phenyl)ethyl)piperidine-1-carboxylate (1.0 g, 2.67 mmol) in DCM (20 mL) was added DAST (1.11 g, 6.67 mmol) at −10° C. After stirring at −10° C. for 15 minutes, the reaction mixture was slowly poured into ice-water (20 mL), and the aqueous layer was extracted with DCM (2×20 mL). The combined organic layers were washed with an aqueous NaHCO₃ solution (20 mL) and brine (10 mL), dried over anhydrous Na₂SO₄, filtered and concentrated to give tert-butyl 4-fluoro-4-(1-(4-(trifluoromethyl)phenyl)ethyl)piperidine-1-carboxylate (1 g, crude), which was used directly in the next reaction.

Step 3

To a solution of tert-butyl 4-fluoro-4-(1-(4-(trifluoromethyl)phenyl)ethyl)piperidine-1-carboxylate (1 g, 2.66 mmol) in dioxane (10 mL) was added HCl/dioxane (6.62 mL, 4 M HCl in dioxane) at 25° C. under nitrogen. The mixture was stirred at 25° C. for 2 hours. The reaction mixture was concentrated to give 4-fluoro-4-(1-(4-(trifluoromethyl)phenyl)ethyl)piperidine hydrochloride (700 mg, crude). LC-ELSD/MS purity 90%, MS ESI calcd. for C₁₄H₁₈F₄N [M+H]⁺ 276.1, found 276.1.

Step 4

2-(Pyrimidin-4-yl)nicotinic acid (611 mg, 3.04 mmol), HATU (1.44 g, 3.81 mmol), DIPEA (1.64 g, 2.21 mmol) and 4-fluoro-4-(1-(4-(trifluoromethyl)phenyl)ethyl)piperidine hydrochloride (700 mg, 2.54 mmol) were combined in DMF (10 mL) and stirred at 25° C. for 2 hours. The mixture was poured into water (20 mL) and extracted with EtOAc (3×10 mL). The combined organic layers were washed with saturated brine (2×20 mL), dried over anhydrous Na₂SO₄, filtered and concentrated. The residue was purified by HPLC (Column: Xtimate C18 150*40 mm*5 um; Condition: A=water (0.04% NH₃H₂O+10 mM NH₄HCO₃)-B=ACN; Begin B: 45%; End B:75%) to afford (800 mg, impure). The residue was purified by HPLC (Column: Phenomenex Gemini-NX C18 75*30 mm*3 um; Condition: A=water (0.225% FA)-B=ACN; Begin B: 40%; End B:70%) to afford racemic-(4-fluoro-4-(1-(4-(trifluoromethyl)phenyl)ethyl)piperidin-1-yl)(2-(pyrimidin-4-yl)pyridin-3-yl)methanone (400 mg, impure). LC-ELSD/MS purity 60%, MS ESI calcd. for C₂₄H₂₃F₄N₄O [M+H]⁺ 459.2, found 459.2. Racemic-(4-fluoro-4-(1-(4-(trifluoromethyl)phenyl)ethyl)piperidin-1-yl)(2-(pyrimidin-4-yl)pyridin-3-yl)methanone (400 mg) was purified by SFC (Column: DAICEL CHIRALCEL OD-H (250 mm*30 mm, Sum); Condition: A=0.1% NH₃H₂O B=EtOH; Begin B: 20%; End B:20%) to afford (S)-(4-fluoro-4-(1-(4-(trifluoromethyl)phenyl)ethyl)piperidin-1-yl)(2-(pyrimidin-4-yl)pyridin-3-yl)methanone (90 mg, impure) and (R)-(4-fluoro-4-(1-(4-(trifluoromethyl)phenyl)ethyl)piperidin-1-yl)(2-(pyrimidin-4-yl)pyridin-3-yl)methanone (170 mg, impure).

The impure (S)-(4-fluoro-4-(1-(4-(trifluoromethyl)phenyl)ethyl)piperidin-1-yl)(2-(pyrimidin-4-yl)pyridin-3-yl)methanone was re-purified by SFC (Column: DAICEL CHIRALPAK IC (250 mm*30 mm, 10 um); Condition: A=Neutral-B=EtOH; Begin B: 40%; End B:40%) to afford (S)-(4-fluoro-4-(1-(4-(trifluoromethyl)phenyl)ethyl)piperidin-1-yl)(2-(pyrimidin-4-yl)pyridin-3-yl)methanone (40.8 mg, 10%). ¹H-NMR (400 MHz, CDCl₃) δ_{H} 8.94-8.68 (m, 3H), 8.31-8.15 (m, 1H), 7.73-7.62 (m, 1H), 7.61-7.51 (m, 2H), 7.48-7.28 (m, 3H), 4.80-4.53 (m, 1H), 3.53-2.82 (m, 5H), 2.27-1.68 (m, 2H), 1.48-1.34 (m, 4H). ¹⁹F-NMR (376.5 MHz, CDCl₃) δ_{F} −62.45, −170.80, −172.18. LC-ELSD/MS purity 99%, MS ESI calcd. for C₂₄H₂₃F₄N₄O [M+H]⁺ 459.3, found 459.3.

The impure (R)-(4-fluoro-4-(1-(4-(trifluoromethyl)phenyl)ethyl)piperidin-1-yl)(2-(pyrimidin-4-yl)pyridin-3-yl)methanone was purified by SFC (Column: DAICEL CHIRALPAK IC (250 mm*30 mm, 10 um); Condition: A=0.1% NH₃H₂O B=EtOH; Begin B: 35%; End B: 35%) to afford (R)-(4-fluoro-4-(1-(4-(trifluoromethyl)phenyl)ethyl)piperidin-1-yl)(2-(pyrimidin-4-yl)pyridin-3-yl)methanone (32.8 mg, 8%). ¹H-NMR (400 MHz, CDCl₃) δ_{H} 8.95-8.68 (m, 3H), 8.29-8.17 (m, 1H), 7.72-7.62 (m, 1H), 7.61-7.51 (m, 2H), 7.49-7.28 (m, 3H), 4.81-4.53 (m, 1H), 3.51-2.82 (m, 4H), 2.30-1.66 (m, 3H), 1.48-1.29 (m, 4H). ¹⁹F-NMR (376.5 MHz, CDCl₃) S_{F}-62.47, −62.52. LC-ELSD/MS purity 99%, MS ESI calcd. for C₂₄H₂₃F₄N₄O [M+H]⁺ 459.3, found 459.3.

### Example 37. Synthesis of [2,4′-bipyridin]-3-yl(4-fluoro-4-((6-(trifluoromethyl)pyridin-3-yl)methyl)piperidin-1-yl)methanone (Cmpd 35)

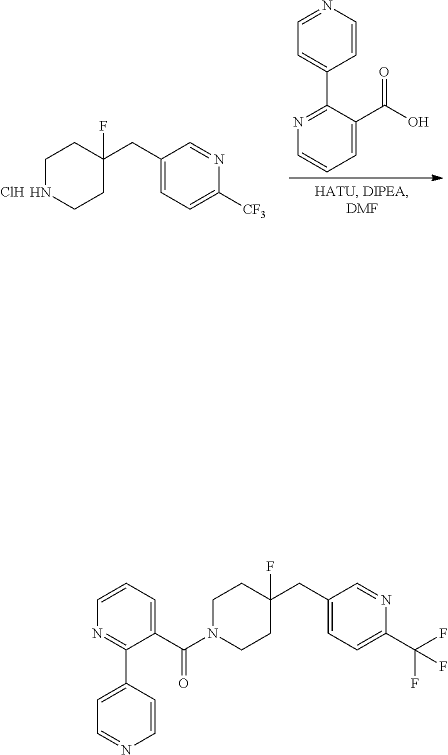 [SMILES: Cl ; FC1(Cc2ccc(C(F)(F)F)nc2)CCNCC1 ; O=C(O)c1cccnc1-c1ccncc1 ; O=C(c1cccnc1-c1ccncc1)N1CCC(F)(Cc2ccc(C(F)(F)F)nc2)CC1]

To a solution of [2,4′-bipyridine]-3-carboxylic acid (0.133 g, 0.669 mmol) and HATU (0.38 g, 1.00 mmol) in DMF (5 mL) was added DIPEA (0.258 mL, 2.0 mmol). 5-((4-fluoropiperidin-4-yl)methyl)-2-(trifluoromethyl)pyridine hydrochloride (0.2 g, 0.6695 mmol) in DMF (5 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, concentrated, and purified by flash column (Column Phenomenex Gemini-NX 80*40 mm*3 um Condition water (0.05% NH₃H₂O)-ACN Begin B 23 End B 53 Gradient Time(min) 8 100% B Hold Time(min) 3 FlowRate (ml/min) 30 Injections 6) to give [2,4′-bipyridin]-3-yl(4-fluoro-4-((6-(trifluoromethyl)pyridin-3-yl)methyl)piperidin-1-yl)methanone (156.0 mg, 53%).

¹H NMR (400 MHz, CDCl₃) δ_{H} 8.83-8.76 (m, 2H), 8.71-8.69 (m, 1H), 8.50-8.42 (m, 1H), 7.82-7.57 (m, 5H), 7.45-7.41 (m, 1H), 4.76-4.51 (m, 1H), 3.09-2.88 (m, 3H), 2.80-2.50 (m, 2H), 1.88-1.73 (m, 1H), 1.50-1.23 (m, 1H), 1.19-1.08 (m, 1H), 1.03-1.01 (m, 1H). ¹⁹F NMR (376.5 MHz, CDCl₃) δ_{F}−67.631, −164.927. LCMS purity >99%, MS ESI calcd. For C₂₃H₂₀F₄N₄O [M+H]⁺ 445.2 found 445.2.

### Example 38. Synthesis of (4-fluoro-4-((6-(trifluoromethyl)pyridazin-3-yl)methyl)piperidin-1-yl)(2-(pyrimidin-4-yl)pyridin-3-yl)methanone (Cmpd 36)

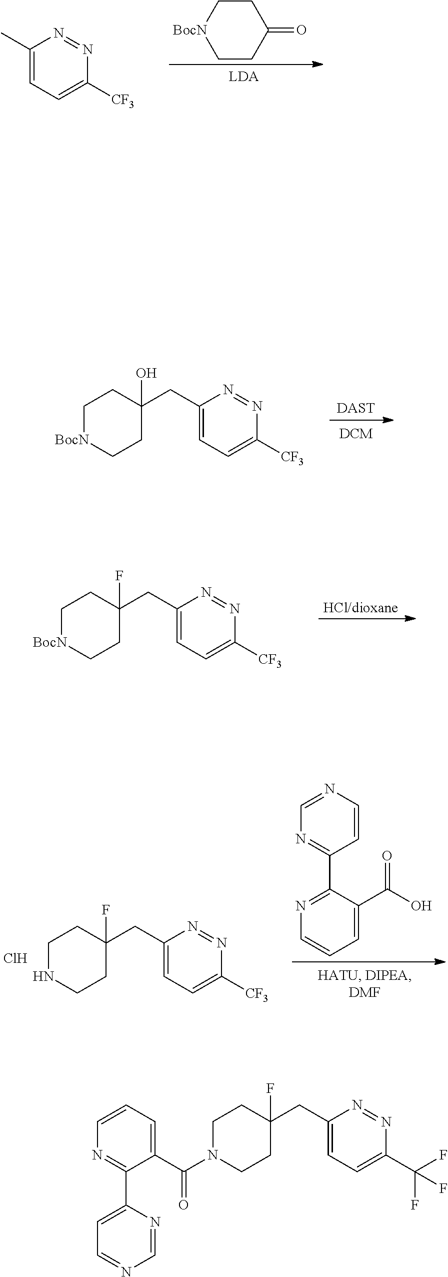

Step 1

To a solution of 3-methyl-6-(trifluoromethyl)pyridazine (800 mg, 4.93 mmol) in dry THF (24 mL) at −78° C. was added LDA (2.58 mL, 5.17 mmol, 2 M in THF) and tert-butyl 4-oxopiperidine-1-carboxylate (1.03 g, 5.17 mmol) and the reaction mixture was stirred over 20 min under N₂ atmosphere. The reaction mixture was poured into ice and sat. NH₄Cl (20 mL). The residue was dissolved in DCM (20 mL), diluted with water (10 mL), and then extracted with DCM (3×20 mL). The combined organic layers were washed with brine (20 mL), dried over anhydrous Na₂SO₄, filtered, the filtrate was concentrated, and the resulting residue was purified by flash column (0˜30% of EtOAc in PE) to give tert-butyl 4-hydroxy-4-((6-(trifluoromethyl)pyridazin-3-yl)methyl)piperidine-1-carboxylate (1.278 g, 63%).

Step 2

To a solution of tert-butyl 4-hydroxy-4-((6-(trifluoromethyl)pyridazin-3-yl)methyl)piperidine-1-carboxylate (1.278 g, 3.51 mmol) in DCM (10 mL) was added DAST (1.13 g, 7.02 mmol) at 0° C. under N₂ atmosphere and the reaction mixture was stirred at 0° C. for 5 min and then concentrated under reduced pressure. The residue was dissolved in DCM (20 mL), diluted with water (10 mL), and then extracted with DCM (3×20 mL). The combined organic layers were washed with brine (20 mL), dried over anhydrous Na₂SO₄, filtered, concentrated, and purified by silica gel column chromatography (50% EtOAc in PE) to give tert-butyl 4-fluoro-4-((6-(trifluoromethyl)pyridazin-3-yl)methyl)piperidine-1-carboxylate (1.27 g).

Step 3

To a solution of tert-butyl 4-fluoro-4-((6-(trifluoromethyl)pyridazin-3-yl)methyl)piperidine-1-carboxylate (1.27 g) in 1,4-dioxane (3 mL) was added hydrogen chloride (3 mL, 7.02 mmol, in 1,4-dioxane) at 25° C. under N₂ atmosphere and the reaction mixture was stirred at 25° C. for 2 h. The reaction mixture was concentrated under reduced pressure to give 3-((4-fluoropiperidin-4-yl)methyl)-6-(trifluoromethyl)pyridazine hydrochloride (1.04 g).

Step 4

To a stirred solution of 3-((4-fluoropiperidin-4-yl)methyl)-6-(trifluoromethyl)pyridazine hydrochloride (1.04 g, crude), 2-(pyrimidin-4-yl)pyridine-3-carboxylic acid (698 mg, 3.47 mmol), HATU (1.38 g, 3.64 mmol) in DMF (10 mL) was added DIEA (1.78 g, 13.8 mmol, 2.39 mL) and the reaction mixture was stirred at 20° C. under N₂ atmosphere for 12 h. The residue was dissolved in EtOAc (20 mL), diluted with water (10 mL), and then extracted with EtOAc. The combined organic layers were washed with brine (20 mL), dried over anhydrous Na₂SO₄, filtered and the filtrate was concentrated and purified by prep-HPLC (Column: Phenomenex Gemini-NX C18 75*30 mm*3 um; Condition: water (0.05% NH₃H₂O+10 mM NH₄HCO₃)-ACN; Begin B: 60; End B: 80; Gradient Time (min):7; 100% B Hold Time (min): 2) to give (4-fluoro-4-((6-(trifluoromethyl)pyridazin-3-yl)methyl)piperidin-1-yl)(2-(pyrimidin-4-yl)pyridin-3-yl)methanone (100 mg, 0.22 mmol, 6%).

¹H NMR (400 MHz, CDCl₃) δ_{H} 9.17 (s, 1H), 8.88 (m, 1H) 8.75 (m, 1H) 8.26 (s, 1H) 7.85-7.75 (m, 1H) 7.61-7.74 (m, 2H) 7.46 (d, J=4.40 Hz, 1H) 4.74-4.47 (m, 1H) 3.51-3.17 (m, 5H) 2.01 (s, 4H). ¹⁹F NMR (376.5 MHz, CDCl₃) δ_{F} −67.015. LCMS purity 95.1%; MS ESI calcd. for C₂₁H₁₈F₄N₆O [M+H]⁺ 447.4, found 447.2.

### Example 39. Synthesis of [2,4′-bipyridin]-3-yl(4-fluoro-4-((6-(trifluoromethyl)pyridazin-3-yl)methyl)piperidin-1-yl)methanone (Cmpd 37)

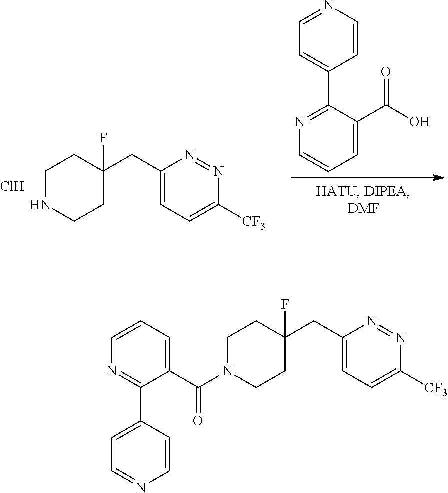 [SMILES: Cl ; FC1(Cc2ccc(C(F)(F)F)nn2)CCNCC1 ; O=C(O)c1cccnc1-c1ccncc1 ; O=C(c1cccnc1-c1ccncc1)N1CCC(F)(Cc2ccc(C(F)(F)F)nn2)CC1]

To a stirred solution of 3-((4-fluoropiperidin-4-yl)methyl)-6-(trifluoromethyl)pyridazine hydrochloride (115 mg), 2-(pyrimidin-4-yl) pyridine-3-carboxylic acid (80.8 mg, 0.402 mmol) and HATU (1.38 g, 3.64 mmol) in DMF (10 mL) was added DIPEA (1.78 g, 13.8 mmol, 2.39 mL). The reaction mixture was stirred at 20° C. under N₂ atmosphere for 12 h. The residue was dissolved in EtOAc (20 mL), diluted with water (10 mL), and then extracted with EtOAc (3×20 mL). The combined organic layers were washed with brine (20 mL), dried over anhydrous Na₂SO₄, filtered and the filtrate was concentrated under reduced pressure. The residue was purified by prep-HPLC (Column: Phenomenex Gemini-NX C18 75*30 mm*3 um; Condition: water (0.05% NH₃H₂O+10 mM NH₄HCO₃)-ACN; Begin B: 60; End B: 80; Gradient Time (min):7; 100% B Hold Time (min): 2) to give [2,4′-bipyridin]-3-yl(4-fluoro-4-((6-(trifluoromethyl)pyridazin-3-yl)methyl)piperidin-1-yl)methanone (2 mg, 0.22 mmol, 7%).

¹H NMR (400 MHz, CDCl₃) δ_{H} 8.90-8.57 (m, 3H), 7.87-7.38 (m, 6H), 4.56 (m, 1H), 3.53-3.29 (m, 1H), 3.23-2.49 (m, 4H), 2.10-1.67 (m, 2H), 1.41-1.22 (m, 2H). ¹⁹F NMR (376.5 MHz, CDCl₃) S_{F}-67.023, −160.709, −162.840. LCMS purity 95.1%; MS ESI calcd. for C₂₂H₁₉F₄N₅O [M+H]+ 445.9, found 446.2.

### Example 40. Synthesis of [2,4′-bipyridin]-3-yl(4-((5-chloropyridin-2-yl)methyl)-4-fluoropiperidin-1-yl)methanone (Cmpd 38)

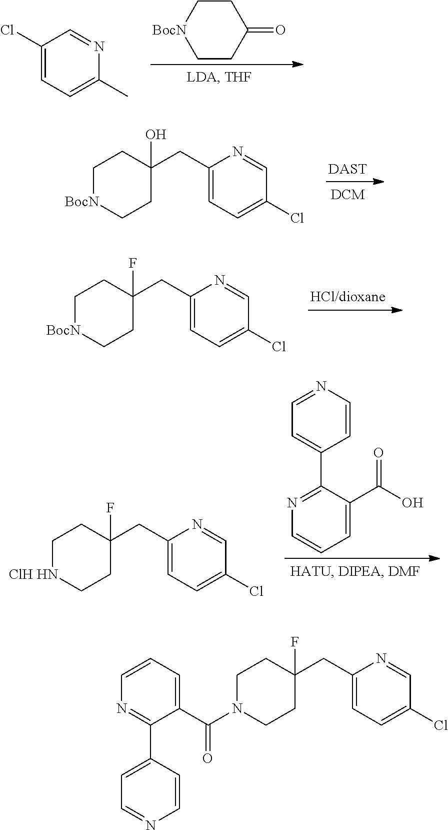

Step 1

To a solution of i-Pr₂NH (12.5 g, 123 mmol) in THE (100 mL) under N₂, n-BuLi (49.2 mL, 2.5 M in hexane, 123 mmol) was added at −78° C. The mixture was stirred at −78° C. for 30 min. 5-chloro-2-methylpyridine (10 g, 78.3 mmol) in THF (100 mL) was added at −75° C. and the mixture was stirred for 1 h. Tert-butyl 4-oxopiperidine-1-carboxylate (18.7 g, 93.9 mmol) was added to the mixture at −75° C. and stirred for 2 h. The reaction mixture was poured into aq. NH₄Cl (400 mL) and extracted with EtOAc (3×100 mL). The combined organic layer was washed with brine (2×100 mL), dried over anhydrous Na₂SO₄, filtered, and concentrated. Purification by flash column (0-20% EtOAc in PE) provided tert-butyl 4-((5-chloropyridin-2-yl)methyl)-4-hydroxypiperidine-1-carboxylate (12 g, 48%).

¹H NMR (400 MHz, CDCl₃) δ_{H} 8.47 (d, J=2.4 Hz, 1H), 7.62 (dd, J=2.4, 8.4 Hz, 1H), 7.08 (d, J=8.4 Hz, 1H), 5.41-5.08 (m, 1H), 3.79 (s, 2H), 3.21 (s, 2H), 2.88 (s, 2H), 1.49 (s, 4H), 1.44 (s, 9H).

Step 2

To a mixture of tert-butyl 4-((5-chloropyridin-2-yl)methyl)-4-hydroxypiperidine-1-carboxylate (6 g, 18.3 mmol) in DCM (100 mL) was added DAST (5.89 g, 36.6 mmol) at 0° C. and the mixture was stirred at 0° C. for 10 min. The residue was poured into ice-water (80 mL) and NaHCO₃ (80 mL) and stirred for 20 min. The aqueous phase was extracted with DCM (3×40 mL). The combined organic phase was washed with brine (2×50 mL), dried over anhydrous Na₂SO₄, filtered and concentrated to give tert-butyl 4-((5-chloropyridin-2-yl)methyl)-4-fluoropiperidine-1-carboxylate (5 g).

Step 3

To a solution of tert-butyl 4-((5-chloropyridin-2-yl)methyl)-4-fluoropiperidine-1-carboxylate (0.8 g, 2.43 mmol) in 1,4-dioxane (5 mL) was added hydrogen chloride (5 mL, 20.0 mmol, 4 M in 1,4-dioxane) at 25° C. under N₂ and the reaction mixture was stirred at 25° C. for 0.5 h. The reaction mixture was concentrated under reduced pressure to give 5-chloro-2-((4-fluoropiperidin-4-yl)methyl)pyridine hydrochloride (lg).

Step 4

To a solution of [2,4′-bipyridine]-3-carboxylic acid (904 mg, 4.52 mmol) and HATU (2.14 g, 5.65 mmol) in DMF (5 mL) was added DIPEA (3.27 mL, 18.8 mmol). 5-chloro-2-((4-fluoropiperidin-4-yl)methyl)pyridine hydrochloride (1 g, 3.77 mmol) in DMF (5 mL) was added slowly. The mixture was stirred at 20° C. for 16 h. The reaction mixture was poured into H₂O (50 mL) and extracted with EtOAc (3×20 mL). The combined organic phase was washed with brine (2×20 mL), dried over anhydrous Na₂SO₄, filtered and purified by HPLC (Column: Phenomenex Gemini-NX 80*40 mm*3 um; Condition: water (0.05% NH₃H₂O)-ACN; Begin B: 18; End B: 48; Gradient Time(min): 8; 100% B Hold Time(min): 2.8; Flow Rate (ml/min): 30; Injections 10) to give [2,4′-bipyridin]-3-yl(4-((5-chloropyridin-2-yl)methyl)-4-fluoropiperidin-1-yl)methanone (106.6 mg, 6.88%).

¹H NMR (400 MHz, CDCl₃) δ_{H} 8.82-8.76 (m, 1H), 8.73-8.68 (m, 2H), 8.50-8.43 (m, 1H), 7.82-7.70 (m, 2H), 7.68-7.62 (m, 1H), 7.60-7.55 (m, 1H), 7.45-7.40 (dd, 1H), 7.30-7.05 (m, 1H) 4.60-4.58 (m, 1H), 3.16-2.93 (m, 3H), 2.90-2.5 (m, 2H), 1.98-1.70 (m, 2H), 1.60-1.26 (m, 2H). 1⁹F NMR (376.5 MHz, CDCl₃) δ_{F} −160.341. LC-ELSD/MS purity 97%, MS ESI calcd. For C₂₂H₂₀ClFN₄O [M+H]⁺ 410.9, found 410.9.

### Example 41. Synthesis of (4-((5-chloro-4-(trifluoromethyl)pyridin-2-yl)methyl)-4-fluoropiperidin-1-yl)(2-(pyrimidin-4-yl)pyridin-3-yl)methanone (Cmpd 39)

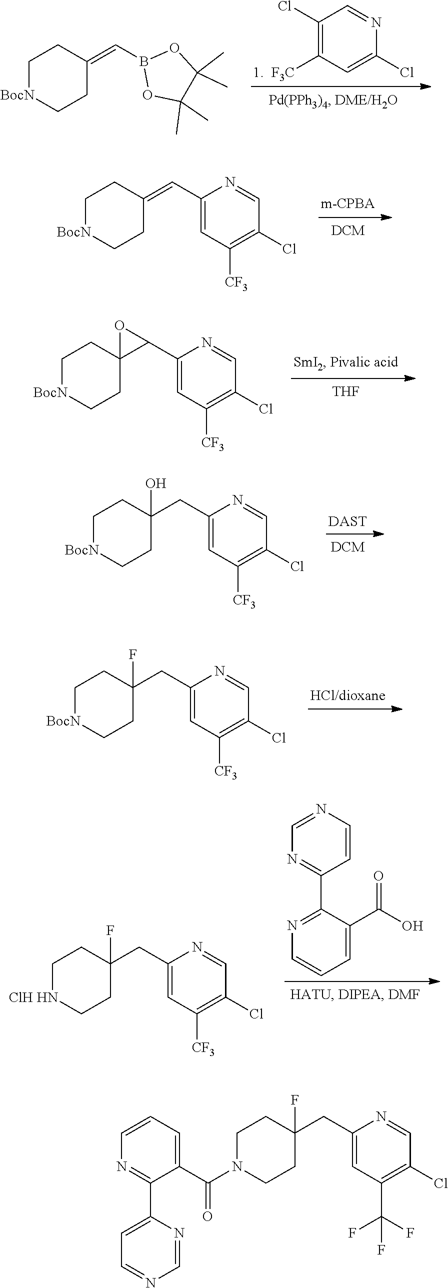

Step 1

A mixture of Pd(PPh₃)₄ (266 mg, 0.231 mmol), Na₂CO₃ (981 mg, 9.26 mmol), 2,5-dichloro-4-(trifluoromethyl)pyridine (1 g, 4.63 mmol) and tert-butyl 4-[(4,4,5,5-tetramethyl-1,3,2-dioxaborolan-2-yl)methylidene]piperidine-1-carboxylate (1.64 g, 5.09 mmol) in DME (16 mL) and water (4 mL) was stirred at 80° C. for 16 hours under N₂. After cooling to 25° C., the mixture was poured into water (15 mL) and extracted with EtOAc (2×15 mL). The combined organic phase was washed with water (15 mL) and brine (15 mL), dried over Na₂SO₄, filtered, and concentrated. The crude product was purified by flash chromatography on silica gel (0% to 1% to 3% to 20% EtOAc in PE) to give tert-butyl 4-((5-chloro-4-(trifluoromethyl)pyridin-2-yl)methylene)piperidine-1-carboxylate (1.2 g, 69%).

¹H NMR (400 MHz, CDCl₃) δ_{H} 8.66 (s, 1H), 7.37 (s, 1H), 6.34 (s, 1H), 3.58-3.45 (m, 4H), 2.95-2.86 (m, 2H), 2.42-2.34 (m, 2H), 1.48 (s, 9H).

Step 2

To a solution of 4-((5-chloro-4-(trifluoromethyl)pyridin-2-yl)methylene)piperidine-1-carboxylate (600 mg, 1.59 mmol) in DCM (10 mL) was added m-CPBA (644 mg, 3.18 mmol) at 0° C. and the reaction mixture was stirred at 0° C. for 2 h. The mixture was diluted with H₂O (10 mL), extracted with DCM (3×10 mL) and the combined organic phase was dried with Na₂SO₄, filtered and concentrated. The residue was purified by flash chromatography on silica gel to afford tert-butyl 2-(5-chloro-4-(trifluoromethyl)pyridin-2-yl)-1-oxa-6-azaspiro[2.5]octane-6-carboxylate (380 mg, 60.8%).

¹H NMR (400 MHz, CDCl₃) δ_{H} 8.71 (s, 1H), 7.56 (s, 1H), 4.06 (s, 1H), 3.80-3.68 (m, 1H), 3.62-3.37 (m, 3H), 1.99-1.89 (m, 1H), 1.77-1.67 (m, 1H), 1.48-1.38 (m, 11H).

Step 3

To a solution of tert-butyl 2-(5-chloro-4-(trifluoromethyl)pyridin-2-yl)-1-oxa-6-azaspiro[2.5]octane-6-carboxylate (530 mg, 1.34 mmol) in HMPA (13 mL) at 20° C. was added 0.1M SmI₂ in THE (80.3 mL, 8.04 mmol). A 0.17M solution of pivalic acid in THE was added (11.8 mL, 2.01 mmol) and the solution was stirred for 48 h. The reaction was quenched with a solution of sodium potassium tartrate (40 mL). The mixture was extracted with diethyl ether (3×20 mL) and the organic layer was washed with H₂O (2×20 mL), dried over Na₂SO₄, and filtered. The solvent was removed in vacuo and the resulting residue was purified by flash chromatography on silica gel to afford tert-butyl 4-((5-chloro-4-(trifluoromethyl)pyridin-2-yl)methyl)-4-hydroxypiperidine-1-carboxylate (260 mg, 49%).

¹H NMR (400 MHz, CDCl₃) δ_{H} 8.67 (s, 1H), 7.42 (s, 1H), 3.91-3.76 (m, 2H), 3.27-3.12 (m, 2H), 2.98 (s, 2H), 1.54-1.49 (m, 4H), 1.45 (s, 9H).

Step 4

To a mixture of tert-butyl 4-((5-chloro-4-(trifluoromethyl)pyridin-2-yl)methyl)-4-hydroxypiperidine-1-carboxylate (200 mg, 0.507 mmol) in DCM (5 mL) was added DAST (162 mg, 1.01 mmol) at 0° C. The mixture was stirred at 0° C. for 5 mins. The residue was poured into ice-water (10 mL) and NaHCO₃ (10 mL) and stirred for 10 min. The aqueous phase was extracted with DCM (3×10 mL). The combined organic phase was washed with brine (2×10 mL), dried over anhydrous Na₂SO₄, filtered, and concentrated to give product. The product was combined with another batch of the same reaction and the mixture was purified by column chromatography (0˜30% of EtOAc in PE) to give tert-butyl 4-((5-chloro-4-(trifluoromethyl)pyridin-2-yl)methyl)-4-fluoropiperidine-1-carboxylate (85 mg, 34%).

¹H NMR (400 MHz, CDCl₃) δ_{H} 8.67 (s, 1H), 7.54 (s, 1H), 3.99-3.87 (m, 2H), 3.20 (s, 1H), 3.14 (s, 1H), 3.10-3.01 (m, 2H), 1.78-1.68 (m, 4H), 1.45 (s, 9H).

Step 5

To a mixture of tert-butyl 4-((5-chloro-4-(trifluoromethyl)pyridin-2-yl)methyl)-4-fluoropiperidine-1-carboxylate (85 mg, 0.214 mmol) in dioxane (2 mL) was added HCl/dioxane (0.535 mL, 4M in dioxane, 2.14 mmol) and the mixture was stirred at 25° C. for 2 h. The mixture was filtered and concentrated to give 5-chloro-2-((4-fluoropiperidin-4-yl) methyl)-4-(trifluoromethyl)pyridine hydrochloride, which was used directly in the next step.

Step 6

To a solution of 2-(pyrimidin-4-yl)pyridine-3-carboxylic acid (47.0 mg, 0.234 mmol), HATU (111 mg, 0.292 mmol) in DMF (3 mL) was added DIPEA (0.170 mL, 0.975 mmol). 5-chloro-2-((4-fluoropiperidin-4-yl)methyl)-4-(trifluoromethyl)pyridine hydrochloride (65.0 mg, 0.195 mmol) in DMF (2 mL) was added slowly. The mixture was stirred at 20° C. for 2 h then poured into H₂O (15 mL) and stirred for 20 min. The aqueous phase was extracted with EtOAc (3×10 mL). The combined organic phase was washed with brine (2×10 mL), dried over anhydrous Na₂SO₄, filtered, and concentrated. Purification by flash column (Column: Phenomenex Gemini-NX 80*40 mm*3 um; Condition: water (0.05% NH₃H₂O)-ACN; Begin B: 46 End B 76; Gradient Time(min) 8 100% B Hold Time(min) 3 FlowRate(ml/min) 30 Injections 4) provided (4-((5-chloro-4-(trifluoromethyl)pyridin-2-yl)methyl)-4-fluoropiperidin-1-yl)(2-(pyrimidin-4-yl)pyridin-3-yl)methanone (26.4 mg, 28%).

¹H NMR (400 MHz, CDCl₃) δ_{H} 9.17-9.01 (m, 1H), 8.89-8.85 (m, 1H), 8.75-8.74 (m, 1H), 8.66 (s, 1H), 8.30-8.20 (m, 1H), 7.77-7.66 (m, 1H), 7.52-7.43 (m, 2H), 4.68-4.53 (m, 1H), 3.43-3.39 (m, 1H), 3.22-3.13 (m, 4H), 1.97-1.94 (m, 2H), 1.75-1.55 (m, 2H). LCMS purity 99%, MS ESI calcd. for C₂₂H₁₈ClF₄N₅O [M+H]⁺ 480.1, found 480.1.

### Example 42. Synthesis of (4-fluoro-4-((5-(trifluoromethyl)pyrazin-2-yl)methyl)piperidin-1-yl)(2-(pyrimidin-4-yl)pyridin-3-yl)methanone (Cmpd 40)

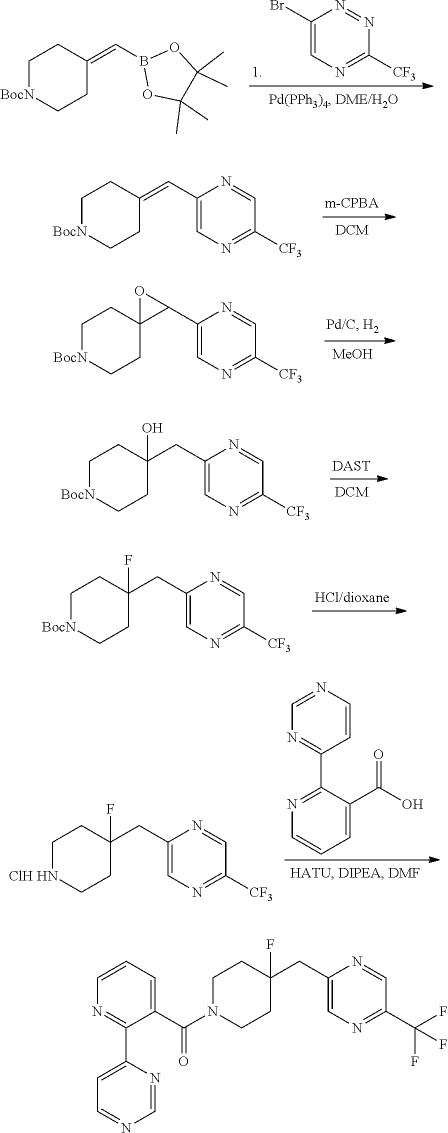

Step 1

A mixture of Pd(PPh₃)₄ (127 mg, 0.11 mmol), Na₂CO₃ (466 mg, 4.40 mmol), 2-bromo-5-(trifluoromethyl)pyrazine (500 mg, 2.20 mmol) and tert-butyl 4-[(4,4,5,5-tetramethyl-1,3,2-dioxaborolan-2-yl)methylidene]piperidine-1-carboxylate (782 mg, 2.42 mmol) in DME (10 mL) and water (2.5 mL) was added to a flask at 20° C. Then the mixture was stirred at 80° C. for 16 h under N₂ and cooled to 20° C. The residue was poured into water (10 mL) and the mixture was extracted with EtOAc (2×10 mL). The combined organic phase was washed with water (10 mL) and brine (10 mL), dried over Na₂SO₄, filtered, and concentrated. Purification by flash chromatography on silica gel (0-20% of EtOAc in PE) gave tert-butyl 4-((5-(trifluoromethyl)pyrazin-2-yl)methylene)piperidine-1-carboxylate (450 mg, 59.6%).

¹H NMR (400 MHz, CDCl₃) δ_{H} 8.95-8.81 (m, 1H), 8.51 (s, 1H), 6.42 (s, 1H), 3.65-3.44 (m, 4H), 2.99 (m, 2H), 2.44 (m, 2H), 1.48 (s, 9H).

Step 2

To a solution of tert-butyl 4-((5-(trifluoromethyl)pyrazin-2-yl)methylene)piperidine-1-carboxylate (100 mg, 0.291 mmol) in DCM (10 mL) was added m-CPBA (352 mg, 1.74 mmol, 85% purity) at 0° C. and the reaction mixture was stirred at 0° C. for 5 hr. The reaction mixture was diluted with Na₂S₂O₃ (10 mL), extracted with DCM (5 ml×2) and the combined organic phase was dried with Na₂SO₄, filtered and concentrated. Purification by flash column (0-20% EtOAc in PE) gave tert-butyl 2-(5-(trifluoromethyl)pyrazin-2-yl)-1-oxa-6-azaspiro[2.5]octane-6-carboxylate (50 mg, 48%).

¹H NMR (400 MHz, CDCl₃) δ_{H} 8.91 (s, 1H), 8.70 (s, 1H), 4.13 (s, 1H), 3.84-3.69 (m, 1H), 3.62-3.49 (m, 2H), 3.47-3.37 (m, 1H), 1.98 (m, 1H), 1.79-1.67 (m, 1H), 1.55 (s, 2H), 1.45 (s, 9H). ¹⁹F NMR (376.5 MHz, CDCl₃) δ_{F}−67.493.

Step 3

To a solution of tert-butyl 2-(5-(trifluoromethyl)pyrazin-2-yl)-1-oxa-6-azaspiro[2.5]octane-6-carboxylate (50 mg, 0.139 mmol) and Pd/C (50 mg, 10% Palladium on carbon) in THE (10 mL) was hydrogenated under 15 psi of hydrogen at 20° C. for 6 hours. The mixture was filtered and washed with THF (30 mL), the filtrate was concentrated to give tert-butyl 4-hydroxy-4-((5-(trifluoromethyl)pyrazin-2-yl)methyl)piperidine-1-carboxylate (60 mg).

Step 4

To a mixture of tert-butyl 4-hydroxy-4-((5-(trifluoromethyl)pyrazin-2-yl)methyl)piperidine-1-carboxylate (60 mg, 0.166 mmol) in DCM (5 mL) was added DAST (53.5 mg, 0.332 mmol) at 0° C. and the mixture was stirred for 10 min. The mixture was poured into ice-water (5 mL) and NaHCO₃ (10 mL) and stirred for 10 min. The aqueous phase was extracted with DCM (2×10 mL) and the combined organic phase was washed with saturated brine (2×20 mL), dried over anhydrous Na₂SO₄, filtered, and concentrated. Purification by flash column (0-10% of EtOAc in PE) provided tert-butyl 4-fluoro-4-((5-(trifluoromethyl)pyrazin-2-yl)methyl)piperidine-1-carboxylate (60 mg).

Step 5

To a solution of tert-butyl 4-fluoro-4-((5-(trifluoromethyl)pyrazin-2-yl)methyl)piperidine-1-carboxylate (60 mg, 0.165 mmol) in 1,4-dioxane (5 mL) was added hydrogen chloride (5 mL, 20.0 mmol, 4 M in 1,4-dioxane) at 20° C. under N₂ and the reaction mixture was stirred at 20° C. for 2 h. The reaction mixture was concentrated to give 2-((4-fluoropiperidin-4-yl)methyl)-5-(trifluoromethyl)pyrazine hydrochloride (60 mg).

Step 6

To a solution of 2-(pyrimidin-4-yl) pyridine-3-carboxylic acid (48.2 mg, 0.24 mmol) and HATU (114 mg, 0.3 mmol) in DMF (3 mL) was added DIPEA (0.174 mL, 1 mmol). 2-((4-fluoropiperidin-4-yl)methyl)-5-(trifluoromethyl)pyrazine hydrochloride (60 mg, 0.2 mmol) in DMF (2 mL) was added slowly. The mixture was stirred at 20° C. for 16 h. The reaction mixture was poured into H₂O (10 mL) and stirred for 20 min. The aqueous phase was extracted with EtOAc (3×10 mL). The combined organic phase was washed with brine (2×10 mL), dried over anhydrous Na₂SO₄, filtered, and concentrated. Purification by HPLC (Column: Phenomenex Gemini-NX 80*40 mm*3 um; Condition: water (0.05NH₃H₂O)-ACN; Begin B: 23; End B: 53; Gradient Time(min): 8; 100% B Hold Time(min): 2; FlowRate(ml/min): 30; Injections: 5) provided (4-fluoro-4-((5-(trifluoromethyl)pyrazin-2-yl)methyl)piperidin-1-yl)(2-(pyrimidin-4-yl)pyridin-3-yl)methanone (7.4 mg, 8%).

¹H NMR (400 MHz, CDCl₃) δ_{H} 9.25-9.10 (m, 1H), 8.95-8.84 (m, 2H), 8.76 (dd, J=1.6, 4.8 Hz, 1H), 8.67 (s, 1H), 8.29 (d, J=4.4 Hz, 1H), 7.76-7.65 (m, 1H), 7.50-7.45 (m, 1H), 4.76-4.51 (m, 1H), 3.53-3.36 (m, 1H), 3.35-3.12 (m, 4H), 2.18-1.84 (m, 2H), 1.69-1.42 (m, 1H), 1.32-1.22 (m, 1H). ¹⁹F NMR (400 MHz, CDCl₃) δ_{F} −67.466, 161.012. LCMS purity 97%, MS ESI calcd. For C₂₁H₁₈F₄N₆O [M+H]⁺ 447.2, found 447.2.

### Example 43. (4-fluoro-4-((2-(trifluoromethyl)pyrimidin-5-yl)methyl)piperidin-1-yl)(2-(pyrimidin-4-yl)pyridin-3-yl)methanone (Cmpd 41)

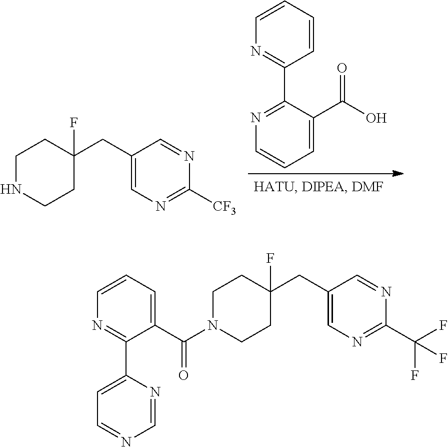 [SMILES: FC1(Cc2cnc(C(F)(F)F)nc2)CCNCC1 ; O=C(O)c1cccnc1-c1ccccn1 ; O=C(c1cccnc1-c1ccncn1)N1CCC(F)(Cc2cnc(C(F)(F)F)nc2)CC1]

5-((4-fluoropiperidin-4-yl)methyl)-2-(trifluoromethyl)pyrimidine was prepared in an analogous manner to 2-((4-fluoropiperidin-4-yl)methyl)-5-(trifluoromethyl)pyrazine in Example 42 using 5-bromo-2-(trifluoromethyl)pyrimidine as starting material.

To a solution of 2-(pyrimidin-4-yl)pyridine-3-carboxylic acid (134 mg, 667 μmol) and HATU (0.19 g, 500 μmol) in DMF (2 mL) was added DIPEA (0.174 mL, 1.0 mmol). 5-((4-fluoropiperidin-4-yl)methyl)-2-(trifluoromethyl)pyrimidine (0.1 g, 0.3336 mmol) in DMF (2 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with brine (2×20 mL), dried over anhydrous Na₂SO₄, filtered, and concentrated. Purification by flash column (Column: Phenomenex Gemini-NX 80*40 mm*3 um; Condition: water (0.05% NH₃H₂O)-ACN; Begin B: 24; End B: 54; Gradient Time (min): 8; 100% B Hold Time(min): 4; FlowRate(ml/min): 30; Injections: 6) provided (4-fluoro-4-((2-(trifluoromethyl)pyrimidin-5-yl)methyl)piperidin-1-yl)(2-(pyrimidin-4-yl)pyridin-3-yl)methanone (37.0 mg, 25.0%).

¹H NMR (400 MHz, CDCl₃) δ ppm 9.20 (s, 1H), 8.90 (s, 1H), 8.77 (s, 3H), 8.30 (s, 1H), 7.69 (m, 1H), 7.43-7.55 (m, 1H), 4.59-4.82 (m, 1H), 3.15-3.56 (m, 3H), 2.90-3.09 (m, 2H), 1.89-2.11 (m, 2H), 1.66-1.88 (m, 2H). LCMS purity 98%, MS ESI calcd. for C₂₁H₁₈F₄N₆O [M+H]⁺ 447.1, found 447.2.

### Example 44. Synthesis of (4-fluoro-4-((5-methylpyridin-2-yl)methyl)piperidin-1-yl)(2-(pyrimidin-4-yl)pyridin-3-yl)methanone (Cmpd 42)

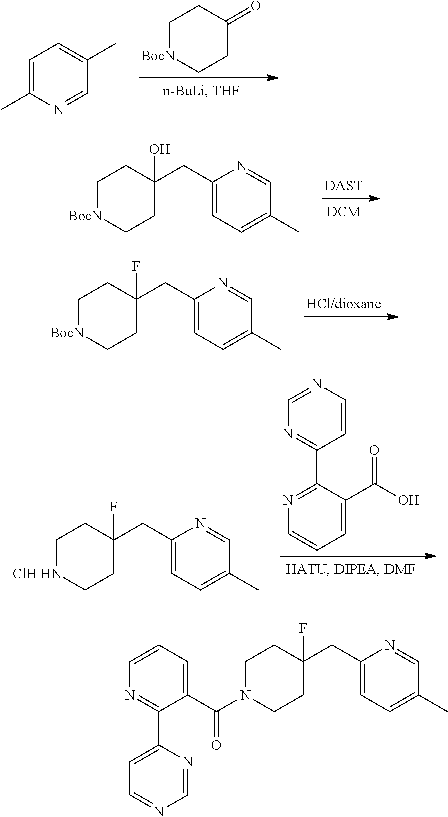

Step 1

To a solution of 2,5-dimethylpyridine (2.0 g, 18.6 mmol) in THE (15 ml) was added n-BuLi (6.68 mL, 2.5 M, 16.7 mmol) dropwise slowly at −70° C. under N₂. After stirring at −70° C. for 0.5 h under N₂, to the mixture was added tert-butyl 4-oxopiperidine-1-carboxylate (2.94 g, 14.8 mmol) in THE (10 ml) at −70° C. under N₂ and stirred for 2 h at −70° C. After warming to 25° C., the residue was poured into water (20 mL) and the mixture was extracted with EtOAc (20 mL×2). The combined organic phase was washed with water (15 mL) and brine (15 mL), dried over Na₂SO₄, filtered, and concentrated. Purification by flash chromatography on silica gel (0% to 10% to 50% EtOAc in PE) provided tert-butyl 4-hydroxy-4-((5-methyl pyridin-2-yl)methyl)piperidine-1-carboxylate (3.5 g, 61.5%).

¹H NMR (400 MHz, CDCl₃) δ_{H} 8.33 (s, 1H), 7.49-7.42 (m, 1H), 7.01 (d, J=7.6 Hz, 1H), 3.86-3.69 (m, 2H), 3.31-3.12 (m, 2H), 2.86 (s, 2H), 2.32 (s, 3H), 1.54-1.45 (m, 4H), 1.45 (s, 9H).

Step 2

To a mixture of tert-butyl 4-hydroxy-4-((5-methyl pyridin-2-yl)methyl)piperidine-1-carboxylate (2 g, 6.52 mmol) in DCM (30 mL) was added DAST (1.57 g, 9.78 mmol) at 0° C. The mixture was stirred at 0° C. for 5 mins. The mixture was poured into ice-water (50 mL) and NaHCO₃ (50 mL), and stirred for 10 min. The aqueous phase was extracted with DCM (3×20 mL). The combined organic phase was washed with saturated brine (2×30 mL), dried over anhydrous Na₂SO₄, filtered, and concentrated to give tert-butyl 4-fluoro-4-((5-methylpyridin-2-yl)methyl)piperidine-1-carboxylate (1.5 g).

¹H NMR (400 MHz, CDCl₃) δ_{H} 8.41-8.34 (m, 1H), 7.53-7.44 (m, 1H), 7.13-7.08 (m, 1H), 3.89 (s, 2H), 3.59-3.42 (m, 4H), 3.15-3.07 (m, 1H), 2.33 (s, 3H), 2.06-1.98 (m, 2H), 1.78-1.69 (m, 1H), 1.46-1.42 (m, 9H).

Step 3

To a mixture of tert-butyl 4-fluoro-4-((5-methylpyridin-2-yl)methyl)piperidine-1-carboxylate (1.5 g, 4.86 mmol) in dioxane (10 mL) was added HCl/dioxane (12.1 mL, 4M in dioxane, 48.6 mmol) and the mixture was stirred at 25° C. for 2 h. The mixture was concentrated to give 2-((4-fluoropiperidin-4-yl)methyl)-5-methylpyridine hydrochloride (1.5 g) which was used directly in the next step.

Step 4

To a solution of 2-(pyrimidin-4-yl)pyridine-3-carboxylic acid (410 mg, 2.04 mmol) and HATU (1.16 g, 3.06 mmol) in DMF (5 mL) was added DIPEA (1.31 g, 10.2 mmol). 2-((4-fluoropiperidin-4-yl)methyl)-5-methylpyridine hydrochloride (0.5 g, 2.04 mmol) in DMF (5 mL) was added slowly. The mixture was stirred at 20° C. for 16 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by HPLC (Column: Phenomenex Gemini-NX 80*40 mm*3 um; Condition: water (0.05% NH₃H₂O)-ACN; Begin B: 22; End B: 52: Gradient Time (min): 8; 100% B Hold Time (min): 3.5; FlowRate (ml/min): 30; Injections: 7) gave the product, which was further purified by SFC (Column: DAICEL CHIRALCEL OJ (250 mm*30 mm, 10 um); Condition: 0.1% NH₃H₂O ETOH; Begin B: 20%; End B: 20%; FlowRate (ml/min): 60; Injections: 25) to give (4-fluoro-4-((5-methylpyridin-2-yl)methyl)piperidin-1-yl)(2-(pyrimidin-4-yl)pyridin-3-yl)methanone (52.9 mg, 6.6%).

¹H NMR (400 MHz, CDCl₃) S_{H} 9.22-8.61 (m, 3H), 8.44-8.33 (m, 1H), 8.30-8.14 (m, 1H), 7.76-7.40 (m, 3H), 7.24-7.09 (m, 1H), 4.72-4.45 (m, 1H), 3.50-3.45 (m, 1H), 3.35-3.09 (m, 4H), 2.45-2.27 (m, 3H), 2.08-1.89 (m, 2H), 1.70-1.64 (m, 2H). ¹⁹F NMR (376.5 MHz, CDCl₃) δ_{F} −159.479. LCMS purity 99%, MS ESI calcd. For C₂₂H₂₂FN₅O [M+H]⁺ 392.2, found 392.0.

### Example 45. Synthesis of [2,4′-bipyridin]-3-yl(4-fluoro-4-((5-methylpyridin-2-yl)methyl)piperidin-1-yl)methanone (Cmpd 43)

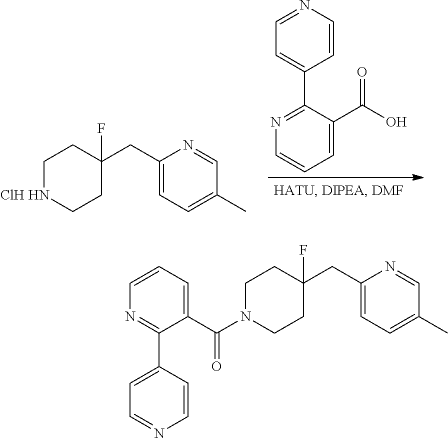

To a solution of [2,4′-bipyridine]-3-carboxylic acid (408 mg, 2.04 mmol) and HATU (1.16 g, 3.06 mmol) in DMF (5 mL) was added DIPEA (1.31 g, 10.2 mmol). 2-((4-fluoropiperidin-4-yl)methyl)-5-methylpyridine hydrochloride (0.5 g, 2.04 mmol) in DMF (5 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by flash column (0˜5% of MeOH in DCM) gave the product, which was further purified by HPLC (Column: Phenomenex Gemini-NX 80*40 mm*3 um; Condition: water (0.05% NH₃H₂O)-ACN; Begin B: 21; End B: 51; Gradient Time(min): 8; 100% B Hold Time(min): 2; FlowRate(ml/min): 30; Injections: 6) then by SFC (Column: DAICEL CHIRALPAK AD (250 mm*30 mm, 10 um); Condition: 0.1% NH₃H₂O ETOH; Begin B: 40%; End B: 40%; FlowRate(ml/min): 70; Injections: 70) to give [2,4′-bipyridin]-3-yl(4-fluoro-4-((5-methylpyridin-2-yl)methyl)piperidin-1-yl)methanone (26 mg, 3%).

¹H NMR (400 MHz, CDCl₃) S_{H} 8.79-8.74 (m, 1H), 8.72-8.60 (m, 2H), 8.38-8.30 (m, 1H), 7.82-7.55 (m, 3H), 7.50-7.34 (m, 2H), 7.19-6.90 (m, 1H), 4.62-4.46 (m, 1H), 3.19-2.95 (m, 2H), 2.90-2.55 (m, 2H), 2.37-2.28 (m, 3H), 1.80-1.65 (m, 2H), 1.54-1.31 (m, 2H), 0.51-0.24 (m, 1H). 1⁹F NMR (376.5 MHz, CDCl₃) δ_{F} −159.939, −161.100 LCMS purity 99%, MS ESI calcd. For C₂₃H₂₃FN₄O [M+H]⁺ 391.2, found 391.1.

### Example 46. Synthesis of (4-fluoro-4-(pyrazin-2-ylmethyl)piperidin-1-yl)(2-(pyrimidin-4-yl)pyridin-3-yl)methanone (Cmpd 44)

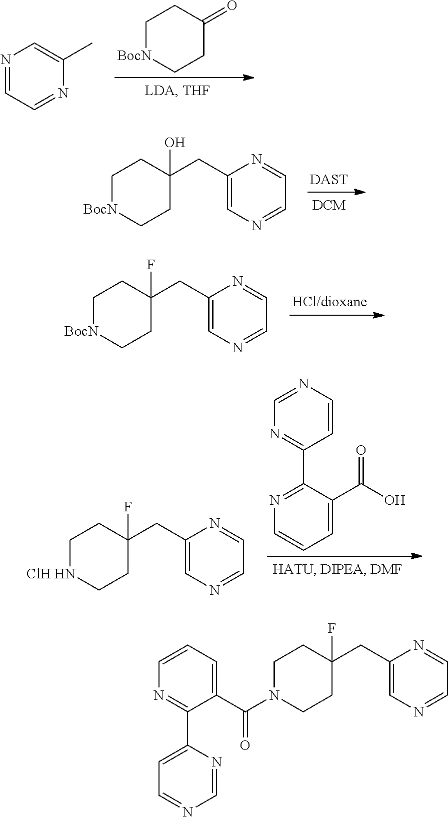

Step 1

To a solution of i-Pr₂NH (11.1 mL, 79.0 mmol) in THE (100 mL) under N₂ was added n-BuLi (31.6 mL, 2.5 M in hexane, 79.0 mmol) at −70° C. The mixture was stirred at −70° C. for 30 min. To the solution of LDA (8.52 g in THF, 79.6 mmol) was added 2-methylpyrazine (5.0 g, 53.1 mmol) in THE (50 mL) and the mixture was stirred at −70° C. for 1 h. Tert-butyl 4-oxopiperidine-1-carboxylate (12.6 g, 63.7 mmol) in THE (50 mL) was added and the mixture was stirred at −70° C. for 3 h. The mixture was poured to saturated ammonium chloride solution (500 mL) and extracted with EtOAc (200 mL×3). The combined organic phase was washed with brine (2×200 mL), dried over Na₂SO₄, filtered, and concentrated. Purification by silica gel chromatography (20-70% EtOAc in PE) provided tert-butyl 4-hydroxy-4-(pyrazin-2-ylmethyl)piperidine-1-carboxylate (10.2 g, 65.8%).

¹H NMR (400 MHz, CDCl₃) δ_{H} 8.57-8.43 (m, 3H), 3.90-3.73 (m, 2H), 3.31-3.14 (m, 2H), 2.96 (s, 2H), 1.56-1.51 (m, 4H), 1.46 (s, 9H).

Step 2

To a mixture of tert-butyl 4-hydroxy-4-(pyrazin-2-ylmethyl)piperidine-1-carboxylate (3.0 g, 10.2 mmol) in DCM (30 mL) was added DAST (3.28 g, 20.4 mmol) at 0° C. The mixture was stirred at 0° C. for 30 min. The mixture was poured into water and NaHCO₃ (80 mL) and stirred for 20 min. The aqueous phase was extracted with DCM (3×30 mL). The combined organic phase was washed with saturated brine (2×20 mL), dried over anhydrous Na₂SO₄, filtered, and concentrated. Purification by column chromatography (EA in PE=10˜30%) provided tert-butyl 4-fluoro-4-(pyrazin-2-ylmethyl)piperidine-1-carboxylate (2.5 g).

¹H NMR (400 MHz, CDCl₃) δ_{H} 8.60-8.41 (m, 3H), 3.90 (m, 1H), 3.87-3.87 (m, 1H), 3.54 (m, 1H), 3.48 (m, 1H), 3.18-3.10 (m, 1H), 3.06 (m, 1H), 2.09-2.04 (m, 1H), 1.80-1.59 (m, 3H), 1.45 (d, J=2.0 Hz, 9H).

Step 3

To a mixture of tert-butyl 4-fluoro-4-(pyrazin-2-ylmethyl)piperidine-1-carboxylate (2.5 g, 8.46 mmol) in dioxane (20 mL) was added HCl/dioxane (21.1 mL, 4M in dioxane, 84.6 mmol). The mixture was stirred at 25° C. for 4 h. The mixture was cooled and concentrated to give 2-((4-fluoropiperidin-4-yl)methyl)pyrazine hydrochloride (2.6 g).

Step 4

To a solution of 2-(pyrimidin-4-yl)pyridine-3-carboxylic acid (0.309 g, 1.54 mmol), HATU (0.733 g, 1.93 mmol) in DMF (5 mL) was added DIPEA (0.675 mL, 3.87 mmol). 2-((4-fluoropiperidin-4-yl)methyl)pyrazine hydrochloride (0.3 g, 1.29 mmol) in DMF (5 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by Prep-HPLC (Column Phenomenex Gemini-NX 80*40 mm*3 um Condition water (0.05% NH₃H₂O)-ACN Begin B 13 End B 43 Gradient Time(min) 8 100% B Hold Time(min) 4 FlowRate(ml/min) 30 Injections 8) provided (4-fluoro-4-(pyrazin-2-ylmethyl)piperidin-1-yl)(2-(pyrimidin-4-yl)pyridin-3-yl) methanone (100 mg) which was further purified by SFC (Column DAICEL CHIRALPAK AD (250 mm*30 mm, 10 um) Condition 0.1% NH₃H₂O ETOH Begin B 35% End B 35% Gradient Time(min) 100% B Hold Time (min) FlowRate (ml/min) 80 Injections to give (4-fluoro-4-(pyrazin-2-ylmethyl)piperidin-1-yl)(2-(pyrimidin-4-yl)pyridin-3-yl)methanone (48.7 mg, 48.7%).

¹H NMR (400 MHz, CDCl₃) δ_{H} 9.22-8.90 (m, 1H), 8.87 (s, 1H), 8.74 (m, 1H), 8.61-8.45 (m, 3H), 8.24 (d, J=5.2 Hz, 1H), 7.73-7.63 (m, 1H), 7.45 (m, 1H), 4.79-4.49 (m, 1H), 3.50-3.36 (m, 1H), 3.26-3.11 (m, 4H), 2.12-1.82 (m, 2H), 1.71-1.57 (m, 2H). ¹⁹F NMR (376.5 MHz, CDCl₃) S_{F}-159.746. LC-ELSD/MS purity >99%, MS ESI calcd. For C₂₀H₁₉FN₆O [M+H]⁺ 379.2, found 379.2.

### Example 47. Synthesis of [2,4′-bipyridin]-3-yl(4-fluoro-4-(pyrazin-2-ylmethyl)piperidin-1-yl) methanone (Cmpd 45)

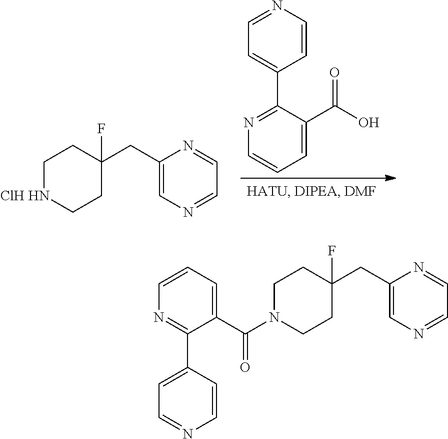

To a solution of [2,4′-bipyridine]-3-carboxylic acid (0.309 g, 1.54 mmol) and HATU (0.733 g, 1.93 mmol) in DMF (5 mL) was added DIPEA (0.675 mL, 3.87 mmol). 2-((4-fluoropiperidin-4-yl)methyl)pyrazine hydrochloride (0.3 g, 1.29 mmol) in DMF (5 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by HPLC (Column: Phenomenex Gemini-NX 80*40 mm*3 um; Condition: water (0.05NH₃H₂O)-ACN; Begin B: 10; End B: 40; Gradient Time(min): 8; 100% B Hold Time(min): 4; FlowRate(ml/min): 30; Injections: 8) provided [2,4′-bipyridin]-3-yl(4-fluoro-4-(pyrazin-2-ylmethyl)piperidin-1-yl)methanone (100 mg), which was further purified by SFC (Column: DAICEL CHIRALPAK AS (250 mm*30 mm, 10 um); Condition: 0.1% NH₃H₂O ETOH; Begin B: 20; End B: 20; FlowRate(ml/min): 60; Injections: 60) to give [2,4′-bipyridin]-3-yl(4-fluoro-4-(pyrazin-2-ylmethyl)piperidin-1-yl)methanone (42.2 mg, 42%).

¹H NMR (400 MHz, CDCl₃) δ_{H} 8.56 (m, 1H), 8.48 (d, J=5.2 Hz, 2H), 8.32-8.15 (m, 3H), 7.59-7.41 (m, 3H), 7.21 (m, 1H), 4.36 (d, J=12.0 Hz, 1H), 2.87-2.55 (m, 4H), 1.95-1.55 (m, 3H), 1.38-1.09 (m, 2H). ¹⁹F NMR (376.5 MHz, CDCl₃) δ_{F} −161.533. LCMS purity >98%, MS ESI calcd. For C₂₁H₂₀FN₅O [M+H]⁺ 378.2, found 378.3.

### Example 48. Synthesis of (4-((6-chloropyridin-3-yl)methyl)-4-fluoropiperidin-1-yl)(2-(pyrimidin-4-yl)pyridin-3-yl)methanone (Cmpd 46)

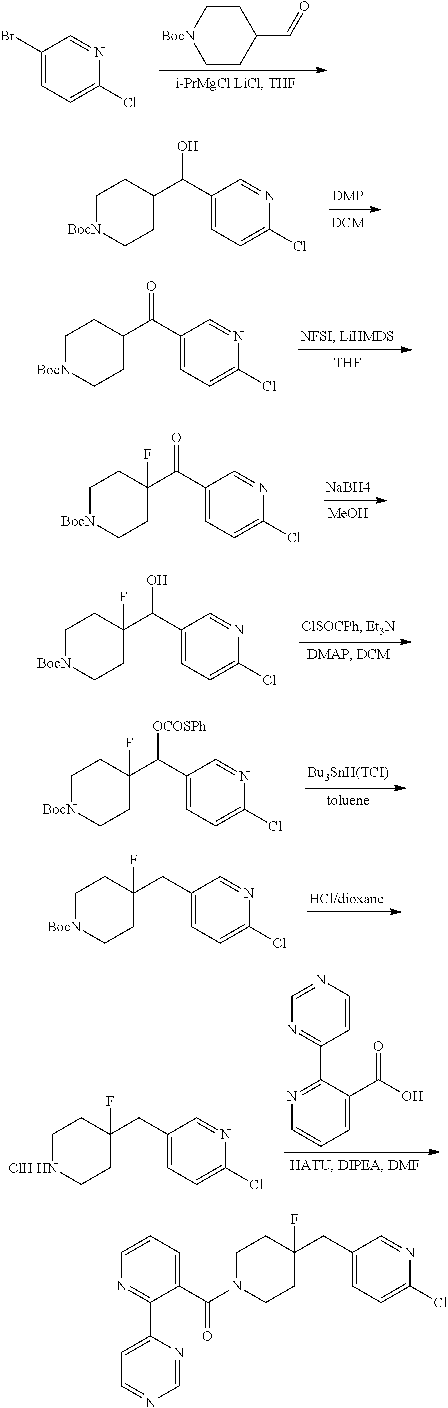

Step 1

A solution of 5-bromo-2-chloropyridine (10 g, 51.9 mmol) in THE (200 mL) was added slowly to a solution of iPrMgCl LiCl (43.8 mL, 1.3 M in hexane, 57.0 mmol) at 0° C. and the mixture was stirred at 0° C. for 30 min. To the 2-chloro-5-(chloromagnesio)pyridine (9 g, in THF, 52.2 mmol) was added a solution of tert-butyl 4-formylpiperidine-1-carboxylate (11.1 g, 52.2 mmol) in THE (150 mL) dropwise slowly at 0° C. under N₂. After stirring at 0° C. for 1 h under N₂ the mixture was poured into water (300 mL) and stirred for 20 min. The aqueous phase was extracted with EtOAc (3×200 mL). The combined organic phase was washed with brine (2×150 mL), dried over anhydrous Na₂SO₄, filtered, and concentrated. Purification by flash column (10˜50% of EtOAc in PE) provided tert-butyl 4-((6-chloropyridin-3-yl)(hydroxy)methyl)piperidine-1-carboxylate (14.5 g, 85.2%).

¹H NMR (400 MHz, CDCl₃) δ_{H} 8.28 (d, J=4.0 Hz, 1H), 7.64 (m, 1H), 7.32 (d, J=8.4 Hz, 1H), 4.47 (d, J=7.2 Hz, 1H), 4.03-4.11 (m, 2H), 2.53-2.69 (m, 2H), 2.32 (s, 1H), 1.67-1.90 (m, 2H), 1.43 (s, 9H), 1.27-1.40 (m, 2H).

Step 2

To a solution tert-butyl 4-((6-chloropyridin-3-yl)(hydroxy)methyl)piperidine-1-carboxylate (2.7 g, 8.26 mmol) in DCM (30 mL) was added DMP (6.99 g, 16.5 mmol) slowly at 0° C. under N₂. The mixture was stirred at 25° C. for 30 min. The mixture was poured into NaHCO₃ (100 mL) and Na₂SO₃ (100 mL) and stirred for 20 min. The aqueous phase was extracted with DCM (3×50 mL). The combined organic phase was washed with brine (2×50 mL), dried over anhydrous Na₂SO₄, filtered, and concentrated to give tert-butyl 4-(6-chloronicotinoyl)piperidine-1-carboxylate (2.4 g, 89.5%).

¹H NMR (400 MHz, CDCl₃) δ_{H} 8.91 (d, J=2.4 Hz, 1H), 8.17 (m, 1H), 7.46 (d, J=8.4 Hz, 1H), 4.15 (s, 2H), 3.26-3.38 (m, 1H), 2.90 (m, 2H), 1.79-1.89 (m, 2H), 1.65-1.78 (m, 2H), 1.45-1.47 (m, 9H)

Step 3

To a solution of tert-butyl 4-(6-chloronicotinoyl)piperidine-1-carboxylate (2 g, 6.15 mmol) in THE (20 mL) was added LiHMDS (9.22 mL, 1M in THF, 9.22 mmol) dropwise slowly at −78° C. under N₂. The mixture was stirred at −78° C. for 30 min. To the mixture was added a solution of NFSI (2.13 g, 6.76 mmol) in THF (20 mL) dropwise at −78° C. for 1 h. The resulting mixture was poured into NaHCO₃ (100 mL) and stirred for 20 min. The aqueous phase was extracted with EtOAc (3×50 mL). The combined organic phase was washed with brine (2×50 mL), dried over anhydrous Na₂SO₄, filtered, and concentrated. Purification by flash column chromatography (0˜20% of EtOAc in PE) provided tert-butyl 4-(6-chloro nicotinoyl)-4-fluoropiperidine-1-carboxylate (1.58 g, 75.2%).

¹H NMR (400 MHz, CDCl₃) δ_{H} 9.06 (d, J=2.0 Hz, 1H), 8.28 (m, 1H), 7.44 (d, J=8.4 Hz, 1H), 4.08 (d, J=6.4 Hz, 2H), 3.19 (t, J=12.0 Hz, 2H), 1.96-2.20 (m, 4H), 1.48 (s, 9H)

Step 4

To a mixture of tert-butyl 4-(6-chloronicotinoyl)-4-fluoropiperidine-1-carboxylate (1.58 g, 4.60 mmol) in EtOH (20 mL) was added NaBH₄ (348 mg, 9.20 mmol) and the mixture was stirred at 25° C. for 10 min. The residue was poured into H₂O (50 mL) and stirred for 20 min. The aqueous phase was extracted with DCM (3×30 mL). The combined organic phase was washed with brine (2×20 mL), dried over anhydrous Na₂SO₄, filtered, and concentrated under reduced pressure to give tert-butyl 4-((6-chloropyridin-3-yl)(hydroxy)methyl)-4-fluoropiperidine-1-carboxylate (1.58 g).

¹H NMR (400 MHz, CDCl₃) δ_{H} 8.32 (d, J=2.4 Hz, 1H), 7.71 (m, 1H), 7.33 (d, J=8.4 Hz, 1H), 4.66 (d, J=13.6 Hz, 1H), 3.87-4.09 (m, 2H), 2.89-3.03 (m, 2H), 1.50-1.85 (m, 4H), 1.42 (s, 9H)

Step 5

To a mixture of tert-butyl 4-((6-chloropyridin-3-yl)(hydroxy)methyl)-4-fluoropiperidine-1-carboxylate (1.58 g, 4.58 mmol) in DCM (20 mL) was added phenyl chloromethanethioate (1.58 g, 9.16 mmol) and DMAP (111 mg, 0.916 mmol) and Et₃N (1.89 mL, 13.7 mmol) at 0° C. The mixture was stirred at 20° C. for 12 h. The reaction mixture was poured into water (50 mL) and stirred for 20 min. The aqueous phase was extracted with EtOAc (3×50 mL). The combined organic phase was washed with brine (2×50 mL), dried over anhydrous Na₂SO₄, filtered and concentrated. Purification by flash column (0-25% of EtOAc in PE) provided tert-butyl 4-((6-chloropyridin-3-yl)(((phenylthio)carbonyl)oxy)methyl)-4-fluoropiperidine-1-carboxylate (1.58 g, 71.8%).

¹H NMR (400 MHz, CDCl₃) δ_{H} 8.42 (d, J=2.0 Hz, 1H), 8.36-8.48 (m, 1H), 7.75 (d, J=8.4 Hz, 1H), 7.36-7.45 (m, 3H), 7.27-7.33 (m, 2H), 7.27-7.33 (m, 1H), 7.05 (d, J=8.0 Hz, 2H), 6.14 (d, J=20.0 Hz, 1H), 6.07-6.19 (m, 1H), 3.87-4.11 (m, 2H), 2.87-3.11 (m, 2H), 1.92-1.94 (m, 1H), 1.50-1.99 (m, 3H), 1.46 (s, 9H)

Step 6

To a solution of tert-butyl 4-((6-chloropyridin-3-yl)(((phenylthio)carbonyl)oxy) methyl)-4-fluoropiperidine-1-carboxylate (750 mg, 1.55 mmol) and tris(monobutyl) tin (1.34 g, 4.65 mmol) and AIBN (50.9 mg, 310 μmol) in toluene (10 mL) at 25° C. under N₂. After stirring at 110° C. for 2 h. The reaction mixture was poured into KF aq. (50 mL) and extracted with EtOAc (2×20 mL). The combined organic phase was washed with brine (2×50 mL), dried over anhydrous Na₂SO₄, filtered, and concentrated. Purification by flash column (0-30% of EtOAc in PE) provided tert-butyl 4-((6-chloropyridin-3-yl)methyl)-4-fluoropiperidine-1-carboxylate (370 mg, 72.6%).

¹H NMR (400 MHz, CDCl₃) δ_{H} 8.20 (d, J=2.0 Hz, 1H), 7.53 (m, 1H), 7.28 (d, J=8.0 Hz, 1H), 3.94 (s, 2H), 2.95-3.08 (m, 2H), 2.80-2.92 (m, 2H), 1.60-1.76 (m, 4H), 1.45 (s, 9H)

Step 7

A solution of tert-butyl 4-((6-chloropyridin-3-yl)methyl)-4-fluoropiperidine-1-carboxylate (370 mg, 1.12 mmol) in HCl/dioxane (10 mL) was stirred at 25° C. for 2 h. The reaction mixture was concentrated under reduced pressure to give 2-chloro-5-((4-fluoropiperidin-4-yl)methyl)pyridine hydrochloride (360 mg).

¹H NMR (400 MHz, CDCl₃) δ_{H} 8.29 (d, J=2.0 Hz, 1H), 7.75 (m, 1H), 7.49 (d, J=8.0 Hz, 1H), 3.19 (d, J=12.4 Hz, 2H), 2.99-3.10 (m, 2H), 2.83-2.96 (m, 2H), 1.74-2.08 (m, 1H), 1.74-2.08 (m, 3H).

Step 8

To a solution of 2-(pyrimidin-4-yl)pyridine-3-carboxylic acid (163 mg, 0.814 mmol) and HATU (384 mg, 1.01 mmol) in DMF (5 mL) was added DIPEA (0.591 mL, 3.39 mmol). 2-chloro-5-((4-fluoropiperidin-4-yl)methyl)pyridine hydrochloride (180 mg, 0.6788 mmol) in DMF (5 mL) was added slowly. The mixture was stirred at 20° C. for 2 h. The reaction mixture poured into H₂O (50 mL) and stirred for 20 min. The aqueous phase was extracted with EtOAc (3×20 mL). The combined organic phase was washed with brine (2×20 mL), dried over anhydrous Na₂SO₄, filtered, and concentrated to give (4-((6-chloropyridin-3-yl)methyl)-4-fluoropiperidin-1-yl)(2-(pyrimidin-4-yl)pyridin-3-yl)methanone (200 mg). The product was purified by HPLC (Column: Phenomenex Gemini-NX 80*40 mm*3 um; Condition: water (0.05% NH₃H₂O)-ACN; Begin B: 24; End B: 54; Gradient Time(min): 8; 100% B Hold Time(min): 2.8; FlowRate(ml/min): 30; Injections 6) to give (4-((6-chloropyridin-3-yl)methyl)-4-fluoropiperidin-1-yl)(2-(pyrimidin-4-yl)pyridin-3-yl)methanone (118.2 mg, 42.2%).

¹H NMR (400 MHz, CDCl₃) δ_{H} 8.98-9.21 (m, 1H), 8.83-8.94 (m, 1H), 8.75 (m, 1H), 8.15-8.33 (m, 2H), 7.67 (d, J=8.0 Hz, 1H), 7.39-7.58 (m, 2H), 7.29 (d, J=8.4 Hz, 1H), 4.55-4.76 (m, 1H), 3.06-3.46 (m, 3H), 2.81-3.00 (m, 2H), 1.76-2.04 (m, 2H), 1.64 (s, 1H), 1.52 (s, 1H) ¹⁹F NMR (376.5 MHz, CDCl₃) δ_{F} −163.09. LCMS purity >98%, calcd. for C₂₁H₁₉ClFN₅O [M+H]⁺ 412.1, found 412.1.

### Example 49. Synthesis of [2,4′-bipyridin]-3-yl(4-((6-chloropyridin-3-yl)methyl)-4-fluoro piperidin-1-yl)methanone (Cmpd 47)

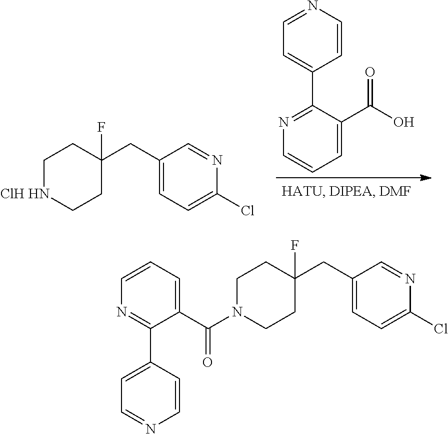

To a solution of [2,4′-bipyridine]-3-carboxylic acid (162 mg, 0.814 mmol) and HATU (384 mg, 1.1 mmol) in DMF (5 mL) was added DIPEA (0.591 mL, 3.39 mmol). 2-chloro-5-((4-fluoropiperidin-4-yl)methyl)pyridine hydrochloride (180 mg, 0.6788 mmol) in DMF (5 mL) was added slowly. The mixture was stirred at 20° C. for 2 h. The reaction mixture poured into H₂O (50 mL) and stirred for 20 min. The aqueous phase was extracted with EtOAc (3×20 mL). The combined organic phase was washed with brine (2×20 mL), dried over anhydrous Na₂SO₄, filtered and concentrated to give [2,4′-bipyridin]-3-yl(4-((6-chloropyridin-3-yl)methyl)-4-fluoropiperidin-1-yl)methanone (200 mg). The product was purified by HPLC (Column: Phenomenex Gemini-NX 80*40 mm*3 um; Condition: water (0.05% NH₃H₂O)-ACN; Begin B: 23; End B: 53; Gradient Time (min): 8; 100% B Hold Time (min): 2.8; FlowRate (ml/min): 30; Injections 76) to give [2,4′-bipyridin]-3-yl(4-((6-chloropyridin-3-yl)methyl)-4-fluoropiperidin-1-yl)methanone (133.2 mg, 47.8%).

¹H NMR (400 MHz, CDCl₃) δ_{H} 8.58-8.89 (m, 3H), 8.07 (s, 1H), 7.54-7.91 (m, 3H), 7.32-7.52 (m, 2H), 7.24 (s, 1H), 4.64 (s, 1H), 2.86-3.05 (m, 3H), 2.30-2.80 (m, 2H), 1.07-1.83 (m, 4H) LCMS purity >99%, calcd. for C₂₂H₂₀ClFN₄O [M+H]⁺ 411.1, found 411.1.

### Example 50. Synthesis of (4-fluoro-4-(pyridazin-3-ylmethyl)piperidin-1-yl)(2-(pyrimidin-4-yl)pyridin-3-yl)methanone (Cmpd 48)

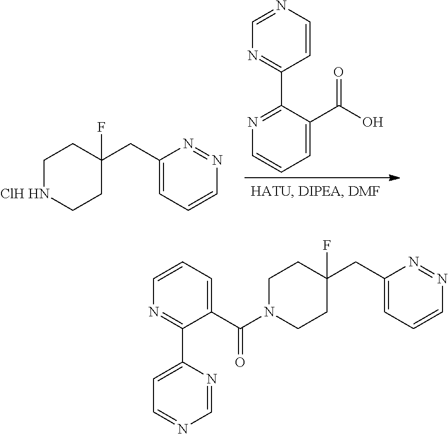

3-((4-fluoropiperidin-4-yl)methyl)pyridazine hydrochloride was prepared according to the analogous procedure in Example 44 using 3-methylpyridazine as starting material.

To a solution of 2-(pyrimidin-4-yl)pyridine-3-carboxylic acid (259 mg, 1.29 mmol) and HATU (733 mg, 1.93 mmol) in DMF (5 mL) was added DIPEA (833 mg, 6.45 mmol). 3-((4-fluoropiperidin-4-yl)methyl)pyridazine hydrochloride (0.3 g, 1.29 mmol) in DMF (5 mL) was added slowly. The mixture was stirred at 20° C. for 16 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated, and combined with another batch of the same reaction. Purification by HPLC (Column: Phenomenex Gemini-NX 80*30 mm*3 um; Condition: water (10 mM NH₄HCO₃)-ACN; Begin B: 0; End B: 60; Gradient Time(min): 9; 100% B Hold Time(min): 2; FlowRate(ml/min): 30; Injections: 5) gave the product. Purification by SFC (Column: DAICEL CHIRALCEL OD-H (250 mm*30 mm, 5 um); Condition: 0.1% NH₃H₂O ETOH; Begin B: 40; End B: 40; FlowRate(ml/min):80) provided (4-fluoro-4-(pyridazin-3-ylmethyl)piperidin-1-yl)(2-(pyrimidin-4-yl)pyridin-3-yl)methanone (6.7 mg, 0.63%).

¹H NMR (400 MHz, CDCl₃) δ_{H} 9.21-8.98 (m, 2H), 8.91-8.83 (m, 1H), 8.78-8.68 (m, 1H), 8.31-8.18 (m, 1H), 7.75-7.61 (m, 1H), 7.55-7.39 (m, 3H), 4.76-4.44 (m, 1H), 3.51-3.32 (m, 3H), 3.29-3.13 (m, 2H), 2.05-1.92 (m, 2H), 1.83-1.54 (m, 2H). LCMS purity 99%, MS ESI calcd. For C₂₀H₁₉FN₆O [M+H]⁺ 379.2, found 379.2.

### Example 51. Synthesis of [2,4′-bipyridin]-3-yl(4-fluoro-4-(pyridazin-3-ylmethyl) piperidin-1-yl)methanone (Cmpd 49)

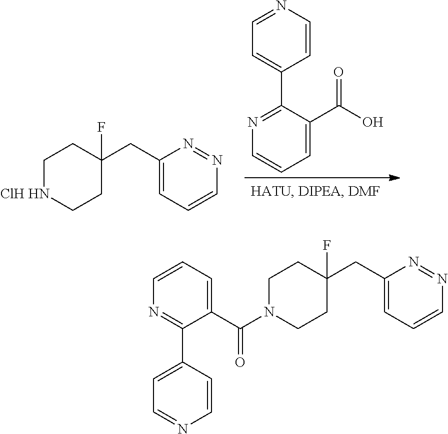

To a solution of [2,4′-bipyridine]-3-carboxylic acid (430 mg, 2.15 mmol) and HATU (1.22 g, 3.22 mmol) in DMF (5 mL) was added DIPEA (1.38 g mL, 10.7 mmol). 3-((4-fluoropiperidin-4-yl)methyl)pyridazine hydrochloride (500 mg, 2.15 mmol) in DMF (5 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by HPLC (Column: Phenomenex Gemini-NX 80*40 mm*3 um; Condition: water (0.05% NH₃H₂O)-ACN: Begin B: 11; End B: 41; Gradient Time(min): 8; 100% B Hold Time(min): 3.5; FlowRate(ml/min): 30; Injections: 7) gave the product, which was combined with the product of another batch of the same reaction. Purification by Prep-TLC (DCM:MeOH=10:1) gave [2,4′-bipyridin]-3-yl(4-fluoro-4-(pyridazin-3-ylmethyl)piperidin-1-yl)methanone (9.1 mg, 0.70%).

¹H NMR (400 MHz, CDCl₃) δ_{H} 9.09 (br s, 1H), 8.85-8.61 (m, 3H), 7.83-7.55 (m, 3H), 7.47-7.29 (m, 3H), 4.64-4.44 (m, 1H), 3.36-2.96 (m, 4H), 2.94-2.49 (m, 1H), 2.17-1.88 (m, 2H), 1.82-1.54 (m, 2H). LCMS purity 99%, MS ESI calcd. For C₂₁H₂₀FN₅O [M+H]⁺ 378.2, found 378.2.

### Example 52. Synthesis of (4-fluoro-4-((5-methylpyrazin-2-yl)methyl)piperidin-1-yl)(2-(pyrimidin-4-yl)pyridin-3-yl)methanone (Cmpd 50)

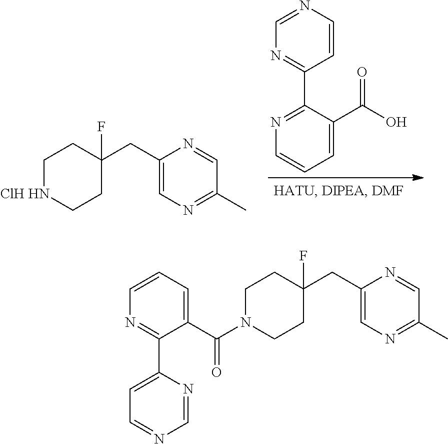

2-((4-fluoropiperidin-4-yl)methyl)-5-methylpyrazine was prepared in an analogous manner to 2-((4-fluoropiperidin-4-yl)methyl)-5-(trifluoromethyl)pyrazine in Example 42 using 2-bromo-5-methylpyrazine as starting material.

To a solution of 2-(pyrimidin-4-yl)pyridine-3-carboxylic acid (147 mg, 0.731 mmol) and HATU (347 mg, 0.915 mmol) in DMF (5 mL) was added DIPEA (0.532 mL, 3.05 mmol, 0.74 g/mL) at 20° C. and stirred for 15 min. 2-((4-fluoropiperidin-4-yl)methyl)-5-methylpyrazine hydrochloride (150 mg, 0.610 mmol) in DMF (5 mL) was added slowly at 20° C. The mixture was stirred at 20° C. for 16 h. The reaction mixture was poured into H₂O (10 mL) and stirred for 20 min. The aqueous phase was extracted with EtOAc (3×10 mL) and washed with saturated brine (2×10 mL), dried over anhydrous Na₂SO₄, filtered, and concentrated. Purification by flash column (0-10% MeOH in DCM) and further purification by SFC (Column: DAICEL CHIRALPAK AD (250 mm*30 mm, 10 um); Condition: 0.1% NH₃H₂O ETOH; Begin B: 40%; End B: 40%; FlowRate(ml/min): 70; Injections: 70) provided (4-fluoro-4-((5-methylpyrazin-2-yl)methyl)piperidin-1-yl)(2-(pyrimidin-4-yl)pyridin-3-yl)methanone (20.9 mg, 29.9%).

¹H NMR (400 MHz, CDCl₃) δ_{H} 9.24-8.83 (m, 2H), 8.74 (d, J=4.0 Hz, 1H), 8.45-8.36 (m, 2H), 8.28-8.20 (m, 1H), 7.71-7.63 (m, 1H), 7.48-7.42 (m, 1H), 4.73-4.50 (m, 1H), 3.47-3.36 (m, 1H), 3.28-3.07 (m, 4H), 2.62-2.51 (m, 3H), 2.10-1.65 (m, 4H). ¹⁹F NMR (376.5 MHz, CDCl₃). δ_{F} −159.649. LCMS purity 99%, MS ESI calcd. For C₂₁H₂₁FN₆O [M+H]⁺ 393.2, found 393.2.

### Example 53. Synthesis of (4-fluoro-4-((6-(fluoromethyl)pyridin-3-yl)methyl)piperidin-1-yl)(2-(pyrimidin-4-yl)pyridin-3-yl)methanone (Cmpd 51)

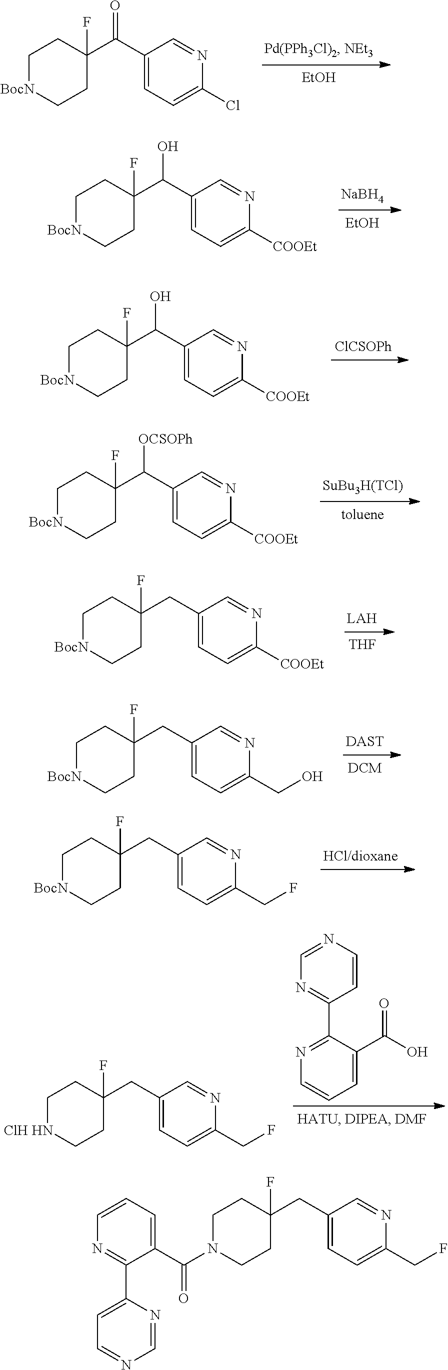

Step 1

A mixture of tert-butyl 4-(6-chloronicotinoyl)-4-fluoropiperidine-1-carboxylate (5 g, 14.0 mmol), Pd(PPh₃)₂Cl₂ (1.01 g, 1.45 mmol), and triethylamine (3.66 g, 36.2 mmol) in EtOH (50 mL) was stirred under carbon monoxide (50 psi) at 80° C. for 16 h. The reaction mixture was filtered. The filtrate was concentrated. Purification by column (0˜20% of EtOAc in PE) provided ethyl 5-(1-(tert-butoxycarbonyl)-4-fluoropiperidine-4-carbonyl)picolinate (1.6 g, 29%).

¹H NMR (400 MHz, CDCl₃) δ_{H} 9.33 (s, 1H), 8.44-8.42 (d, J=8.0 Hz, 1H), 8.20 (d, J=8.0 Hz, 1H), 4.53-4.47 (m, 2H), 4.13-4.08 (m, 2H), 3.22-3.16 (m, 2H), 2.16-2.00 (m, 4H), 1.47 (s, 9H), 1.45-1.43 (m, 3H).

Step 2

To a solution of ethyl 5-(1-(tert-butoxycarbonyl)-4-fluoropiperidine-4-carbonyl)picolinate (1.6 g, 4.20 mmol) in EtOH (20 mL) was added NaBH₄ (317 mg, 8.40 mmol). The mixture was stirred at 25° C. for 10 min. The mixture was poured into H₂O (50 mL) and stirred for 20 min. The aqueous phase was extracted with DCM (3×50 mL). The combined organic phase was washed with brine (2×50 mL), dried over anhydrous Na₂SO₄, filtered and concentrated under reduced pressure to give ethyl 5-((1-(tert-butoxycarbonyl)-4-fluoropiperidin-4-yl)(hydroxy)methyl)picolinate (1.6 g).

¹H NMR (400 MHz, CDCl₃) δ_{H} 8.68 (s, 1H), 8.13-8.11 (m, 1H), 7.89-7.87 (m, 1H), 4.77-4.74 (m, 1H), 4.50-4.45 (m, 2H), 4.08-3.99 (m, 2H), 2.95-2.91 (m, 3H), 1.70-1.67 (m, 2H), 1.54-1.65 (m, 2H), 1.43-1.47 (m, 3H), 1.42 (s, 9H).

Step 3

To a solution of ethyl 5-((1-(tert-butoxycarbonyl)-4-fluoropiperidin-4-yl) (hydroxy)methyl)picolinate (1.6 g, 4.18 mmol) in DCM (20 mL) was added phenyl chloromethanethioate (1.44 g, 8.36 mmol), DMAP (102 mg, 0.836 mmol) and Et₃N (1.72 mL, 12.5 mmol) at 0° C. The mixture was stirred at 20° C. for 12 h, then poured into water (50 mL) and stirred for 20 mins. The mixture was extracted with DCM (3×50 mL). The combined organic phase was washed with brine (2×50 mL), dried over anhydrous Na₂SO₄, filtered and concentrated and purified by flash column (0-30% of EtOAc in PE) to give ethyl 5-((1-(tert-butoxycarbonyl)-4-fluoropiperidin-4-yl)((phenoxycarbonothioyl)oxy)methyl)picolinate (1.13 g, 52%).

¹H NMR (400 MHz, CDCl₃) δ_{H} 8.81 (s, 1H), 8.23-8.21 (d, J=8.0 Hz, 1H), 8.00-7.98 (d, J=8.0 Hz, 1H), 7.42-7.38 (m, 2H), 7.31-7.26 (m, 1H), 7.05-7.03 (m, 2H), 6.23-6.18 (m, 1H), 4.54-4.48 (m, 2H), 4.09-4.02 (m, 2H), 3.06-2.95 (m, 2H), 1.79-1.60 (m, 4H), 1.36-1.48 (m, 12H).

Step 4

A solution of ethyl 5-((1-(tert-butoxycarbonyl)-4-fluoropiperidin-4-yl) ((phenoxycarbonothioyl)oxy)methyl)picolinate (480 mg, 0.926 mmol) and tris(monobutyl) tin (1.07 g, 3.70 mmol) and AIBN (30.3 mg, 0.19 mmol) in toluene (40 mL) was stirred at 110° C. for 2 h. The reaction mixture was poured into KF aq. (50 mL) and extracted with EtOAc (2×50 mL). The combined organic phase was washed with brine (2×50 mL), dried over anhydrous Na₂SO₄, filtered and concentrated and purified by flash column (0˜25% of EtOAc in PE) to give ethyl 5-((1-(tert-butoxycarbonyl)-4-fluoropiperidin-4-yl)methyl)picolinate (60 mg, 18%).

¹H NMR (400 MHz, CDCl₃) δ_{H} 8.59 (s, 1H), 8.11-8.09 (d, J=8.0 Hz, 1H), 7.74-7.72 (d, J=8.0 Hz, 1H), 4.51-4.46 (m, 2H), 3.94 (s, 2H), 2.95-3.04 (m, 4H), 1.70-1.67 (m, 4H), 1.43-1.47 (m, 12H).

Step 5

To a solution of ethyl 5-((1-(tert-butoxycarbonyl)-4-fluoropiperidin-4-yl)methyl)picolinate (60 mg, 0.164 mmol) in THE (3 mL) was added LiAlH₄ (9.29 mg, 0.245 mmol). After stirring at 0° C. for 0.5 h. The mixture was quenched with H₂O (1 mL) slowly and NaOH (1 ml, 15% aq.) and H₂O (3 mL), filtered, and concentrated to give tert-butyl 4-fluoro-4-((6-(hydroxymethyl)pyridin-3-yl)methyl)piperidine-1-carboxylate (35 mg).

¹H NMR (400 MHz, CDCl₃) δ_{H} 8.40 (s, 1H), 7.61-7.59 (d, J=8.0 Hz, 1H), 7.26-7.24 (d, J=8.0 Hz, 1H), 4.77 (s, 2H), 3.85-3.92 (m, 2H), 3.04-2.97 (m, 2H), 2.94-2.89 (d, J=20 Hz, 2H), 1.63-1.79 (m, 4H), 1.45 (s, 9H).

Step 6

To a solution of tert-butyl 4-fluoro-4-((6-(hydroxymethyl)pyridin-3-yl)methyl) piperidine-1-carboxylate (100 mg, 0.308 mmol) in DCM (5 mL) was added DAST (99.2 mg, 0.616 mmol) at 0° C. The mixture was stirred at 0° C. for 10 min. The mixture was poured into water (5 mL) and NaHCO₃ (10 mL) and stirred for 10 min. The mixture was extracted with DCM (2×10 mL). The combined organic phase was washed with brine (2×20 mL), dried over anhydrous Na₂SO₄, filtered, concentrated and purified by flash column (0-35% of EtOAc in PE) to give tert-butyl 4-fluoro-4-((6-(fluoromethyl)pyridin-3-yl) methyl)piperidine-1-carboxylate (75 mg).

¹H NMR (400 MHz, CDCl₃) δ_{H} 8.42 (s, 1H), 7.70-7.68 (m, 1H), 7.48-7.46 (m, 1H), 5.59-5.47 (m, 2H), 3.94 (s, 2H), 3.02-2.99 (m, 2H), 2.96-2.90 (m, 2H), 1.70-1.62 (m, 4H), 1.45 (s, 9H).

Step 7

A solution of tert-butyl 4-fluoro-4-((6-(fluoromethyl)pyridin-3-yl)methyl) piperidine-1-carboxylate (75 mg, 0.230 mmol) in HCl/dioxane (4 M, 10 mL) was stirred at 25° C. for 2 h. The reaction mixture was concentrated under reduced pressure to give 2-(fluoromethyl)-5-((4-fluoropiperidin-4-yl)methyl)pyridine hydrochloride (80 mg).

Step 8

To a solution of 2-(pyrimidin-4-yl)pyridine-3-carboxylic acid (36.6 mg, 0.182 mmol), HATU (86.6 mg, 0.228 mmol) in DMF (2 mL) was added DIPEA (0.132 mL, 0.761 mmol). Then 2-(fluoromethyl)-5-((4-fluoropiperidin-4-yl)methyl)pyridine hydrochloride (40 mg, 0.1522 mmol) in DMF (2 mL) was dropwise slowly. The mixture was stirred at 20° C. for 2 h and poured into H₂O (20 mL) and stirred for 20 min. The aqueous phase was extracted with EtOAc (3×10 mL). The combined organic phase was washed with brine (2×10 mL), dried over anhydrous Na₂SO₄, filtered, and concentrated. Purification by HPLC (Column:Phenomenex Gemini-NX 80*40 mm*3 um; Condition: water (0.05% NH₃H₂O)-ACN; Begin B: 21; End B: 51; Gradient Time(min): 8; 100% B Hold Time(min): 2.5; FlowRate(ml/min): 30; Injections 4) provided (4-fluoro-4-((6-(fluoromethyl)pyridin-3-yl)methyl)piperidin-1-yl)(2-(pyrimidin-4-yl)pyridin-3-yl)methanone (7.6 mg, 12%).

¹H NMR (400 MHz, CDCl₃) δ_{H} 9.18-8.81 (m, 2H), 8.75-8.73 (m, 1H), 8.40 (s, 1H), 8.24-8.22 (m, 1H), 7.59-7.67 (m, 2H), 7.46-7.41 (m, 2H), 5.56-5.41 (m, 2H), 4.57-4.71 (m, 1H), 3.41-3.21 (m, 3H), 3.18-2.92 (m, 2H), 1.94-1.73 (m, 2H), 1.56-1.45 (m, 2H). ¹⁹F NMR (376.5 MHz, CDCl₃) δ_{F} −161.98, −220.66. LCMS purity 97%, calcd. for C₂₂H₂₁F₂N₅O [M+H]⁺ 410.2, found 410.2.

### Example 54. Synthesis of [2,4′-bipyridin]-3-yl(4-fluoro-4-((5-methylpyrazin-2-yl)methyl)piperidin-1-yl)methanone (Cmpd 52)

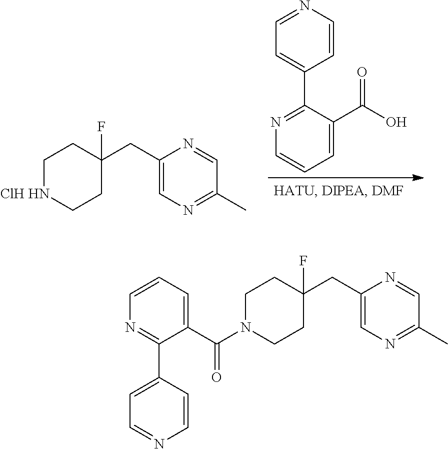

To a solution of [2,4′-bipyridine]-3-carboxylic acid (78.0 mg, 0.39 mmol), HATU (185 mg, 0.487 mmol) in DMF (3 mL) was added DIPEA (0.282 mL, 1.62 mmol) at 20° C. 2-((4-fluoropiperidin-4-yl)methyl)-5-methylpyrazine hydrochloride (80 mg, 0.325 mmol) in DMF (2 mL) was added slowly. The mixture was stirred at 20° C. for 16 h. The reaction mixture was poured was into H₂O (10 mL) and stirred for 20 min. The aqueous phase was extracted with EtOAc (3×10 mL) and washed with saturated brine (2×10 mL), dried over anhydrous Na₂SO₄, filtered, and concentrated. Purification by flash column (0-10% MeOH in DCM) and further purification by HPLC (Column: Phenomenex C18 80*40 mm*3 um; Condition: water (NH₃H₂O)-ACN; Begin B: 15; End B: 45; Gradient Time(min): 8; 100% B Hold Time(min): 2; FlowRate(ml/min): 30; Injections: 5) provided [2,4′-bipyridin]-3-yl(4-fluoro-4-((5-methylpyrazin-2-yl)methyl)piperidin-1-yl)methanone (13.9 mg, 19.6%).

¹H NMR (400 MHz, CDCl₃) δ_{H} 8.78 (dd, J=1.6, 4.8 Hz, 1H), 8.69 (s, 2H), 8.43-8.19 (m, 2H), 7.80-7.67 (m, 2H), 7.65-7.54 (m, 1H), 7.42 (dd, J=4.8, 7.6 Hz, 1H), 4.64-4.50 (m, 1H), 3.15-2.93 (m, 3H), 2.89-2.62 (m, 2H), 2.59-2.49 (m, 4H), 1.95-1.75 (m, 1H), 1.56-1.27 (m, 2H). 1⁹F NMR (376.5 MHz, CDCl₃). δ_{F} −161.592. LCMS purity 99%, MS ESI calcd. For C₂₂H₂₂FN₅O [M+H]⁺ 392.2, found 392.2.

### Example 55 and 56. Synthesis of (4-fluoro-4-((6-methylpyridin-3-yl)methyl)piperidin-1-yl)(2-(pyrimidin-4-yl)pyridin-3-yl)methanone (Cmpd 53) and Synthesis of [2,4′-bipyridin]-3-yl(4-fluoro-4-((6-methylpyridin-3-yl)methyl)piperidin-1-yl)methanone (Cmpd 57)

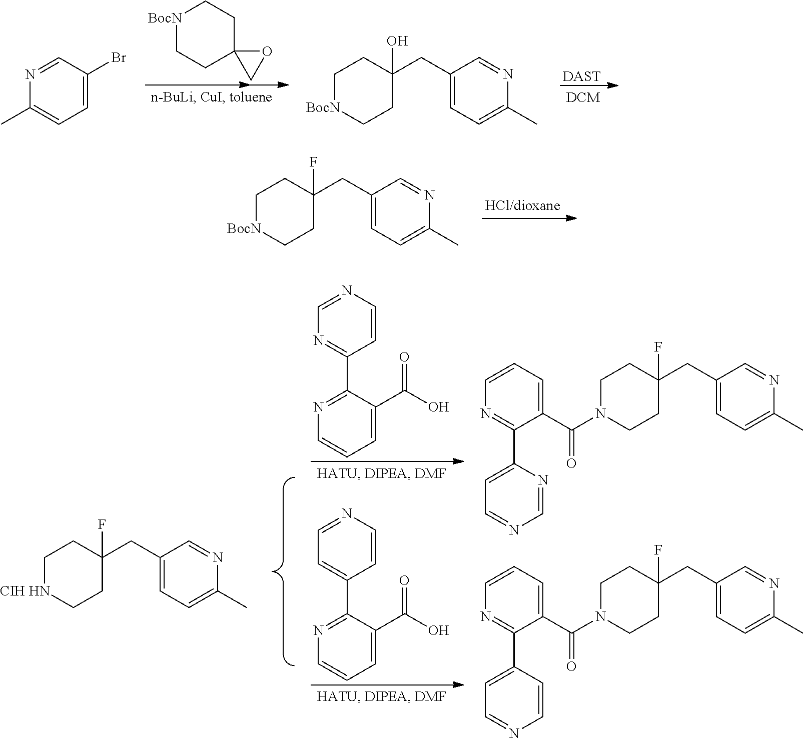

Step 1

To a solution of 5-bromo-2-methylpyridine (5 g, 29.0 mmol) in toluene (50 mL) was added dropwise n-BuLi (12.7 mL) at −70° C. for 0.5 h and then was added CuI (6.07 g, 31.9 mmol) in one portion. The reaction mixture was stirred at −70° C. for 0.5 h. Then to the mixture was added dropwise tert-butyl 1-oxa-6-azaspiro [2.5] octane-6-carboxylate (7.42 g, 34.8 mmol) in THF (30 mL). The reaction mixture was stirred at −70° C. for 4 h. The mixture was warmed to room temperature, poured into sat. NH₄Cl, extracted with DCM, dried over anhydrous Na₂SO₄, filtered and concentrated. The residue was purified by flash column (3-10% of MeOH in DCM) to afford tert-butyl 4-hydroxy-4-((6-methylpyridin-3-yl)methyl)piperidine-1-carboxylate.

Step 2

To a solution of tert-butyl 4-hydroxy-4-((6-methylpyridin-3-yl)methyl)piperidine-1-carboxylate (900 mg, 2.93 mmol) in DCM (2 mL) was added dropwise DAST (944 mg, 5.86 mmol) at 0° C. under N₂. The mixture was stirred at 0° C. for 10 min. The mixture was poured into NaHCO₃ (30 mL) and stirred for 20 min. The aqueous phase was extracted with DCM (2×40 mL). The combined organic phase was washed with saturated brine (2×30 mL), dried over anhydrous Na₂SO₄, filtered and concentrated. The residue was purified by silica gel chromatography (3˜10% of EtOAc in PE) to afford tert-butyl 4-fluoro-4-((6-methylpyridin-3-yl)methyl)piperidine-1-carboxylate (640 mg, 2.07 mmol), which was used directly in the next step.

Step 3

To a solution of tert-butyl 4-fluoro-4-((6-methylpyridin-3-yl)methyl)piperidine-1-carboxylate (600 mg, 1.94 mmol) in dioxane (5 mL) was added HCl/dioxane (4N, 4.85 mL) and the reaction was stirred at 15° C. for 1 h. The reaction mixture was concentrated to give 5-((4-fluoropiperidin-4-yl)methyl)-2-methylpyridine hydrochloride (600 mg), which was used directly in the next step.

Step 4.

To a solution of 2-(pyrimidin-4-yl)pyridine-3-carboxylic acid (1.1 g, 5.50 mmol), HATU (2.09 g, 5.05 mmol) in DMF (5 mL) was added DIPEA (1.42 g, 11 mmol). 5-((4-fluoropiperidin-4-yl)methyl)-2-methylpyridine hydrochloride (900 mg, 3.67 mmol) in DMF (2 mL) was added slowly. The mixture was stirred at 20° C. for 2 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered and concentrated to give (4-fluoro-4-((6-methylpyridin-3-yl)methyl)piperidin-1-yl)(2-(pyrimidin-4-yl)pyridin-3-yl)methanone (600 mg). Purification by HPLC (Column: Phenomenex Gemini-NX 80*40 mm*3 um; Condition: water (0.05NH₃H₂O)-CAN; Begin B: 21; End B: 51; Gradient Time(min): 8; 100% B Hold Time(min): 4; Flow Rate (ml/min): 30; Injections 12) provided (4-fluoro-4-((6-methylpyridin-3-yl)methyl)piperidin-1-yl)(2-(pyrimidin-4-yl)pyridin-3-yl)methanone (96 mg, 0.2452 mmol). Further purification by HPLC (Column: DAICEL CHIRALPAK IG (250 mm*30 mm, 10 um); Condition: 0.1% NH₃H₂O ETOH; Begin B: 60%; End B: 60%; FlowRate(ml/min): 80; Injections: 70) followed by lyophilization provided (4-fluoro-4-((6-methylpyridin-3-yl)methyl)piperidin-1-yl)(2-(pyrimidin-4-yl)pyridin-3-yl)methanone (10 mg, 0.0256 mmol).

¹HNMR (400 MHz, CDCl₃) δ ppm 9.11-9.29 (m, 1H), 8.88 (d, J=5.2 Hz, 1H), 8.76 (m, 1H), 8.54 (d, J=4.0 Hz, 1H), 8.20-8.32 (m, 1H), 7.65-7.73 (m, 1H), 7.58-7.65 (m, 1H), 7.44-7.49 (m, 1H), 7.12-7.16 (m, 1H), 4.53-4.75 (m, 1H), 3.46 (d, J=7.6 Hz, 1H), 3.17-3.28 (m, 2H), 2.87-3.00 (m, 2H), 2.03-2.21 (m, 3H), 1.65-1.97 (m, 4H). ¹⁹F NMR (376.5 MHz, CDCl₃)^{6F}. −163.50, −163.87. LCMS purity >98%, calcd. for C₂₂H₂₂FN₅O [M+H]⁺ 392.2, found 392.0.

Step 5.

To a solution of [2,4′-bipyridine]-3-carboxylic acid (0.117 g, 0.588 mmol) and HATU (0.279 g, 0.735 mmol) in DMF (5 mL) was added DIPEA (0.255 mL, 1.47 mmol). 5-((4-fluoropiperidin-4-yl)methyl)-2-methylpyridine hydrochloride (0.12 g, 0.4901 mmol) in DMF (5 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered and concentrated. Purification by Prep-HPLC (Column: Phenomenex Gemini-NX 80*40 mm*3 um; Condition: water (0.05% NH₃H₂O)-ACN; Begin B: 19; End B: 49; Gradient Time(min): 8; 100% B Hold Time(min): 3; FlowRate(ml/min): 30; Injections: 5) provided [2,4′-bipyridin]-3-yl(4-fluoro-4-((6-methylpyridin-3-yl)methyl)piperidin-1-yl)methanone (32.9 mg, 17.2%). 1HNMR (400 MHz, CDCl₃) δ 8.86-8.75 (m, 1H), 8.71 (d, J=6.0 Hz, 1H), 8.50 (d, J=2.4 Hz, 1H), 7.82-7.70 (m, 2H), 7.67-7.54 (m, 2H), 7.43 (m, 1H), 7.11 (m, 2H), 4.68-4.51 (m, 1H), 3.26-2.93 (m, 2H), 2.91-2.77 (m, 1H), 2.74-2.61 (m, 2H), 2.15-1.73 (m, 2H), 1.70-1.55 (m, 3H), 1.52-1.18 (m, 2H). LCMS purity >95%, calcd. for C₂₃H₂₃FN₄O [M+H]⁺ 391.2, found 391.2.

### Example 57. Synthesis of [2,4′-bipyridin]-3-yl(4-fluoro-4-((6-(fluoromethyl)pyridin-3-yl)methyl)piperidin-1-yl)methanone (Cmpd 54)

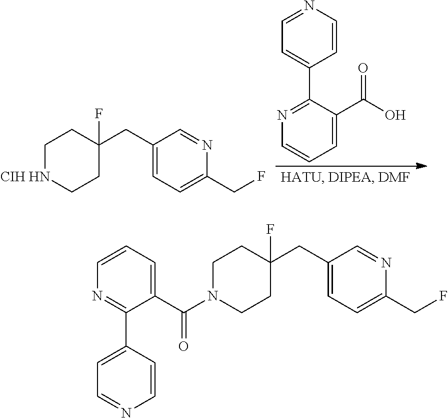

To a solution of [2,4′-bipyridine]-3-carboxylic acid (36.4 mg, 0.182 mmol) and HATU (86.6 mg, 228 μmol) in DMF (2 mL) was added DIPEA (0.591 mL, 3.39 mmol). 2-(fluoromethyl)-5-((4-fluoropiperidin-4-yl)methyl)pyridine hydrochloride (40 mg, 0.1522 mmol) in DMF (2 mL) was added slowly. The mixture was stirred at 20° C. for 2 h. The reaction mixture poured into H₂O (10 mL) and stirred for 20 min. The aqueous phase was extracted with EtOAc (3×10 mL). The combined organic phase was washed with brine (2×10 mL), dried over anhydrous Na₂SO₄, filtered and concentrated. Purification by HPLC (Column:Phenomenex Gemini-NX 80*40 mm*3 um; Condition: water (0.05% NH₃H₂O)-ACN; Begin B: 19; End B: 49; Gradient Time(min): 8; 100% B Hold Time(min): 2.5; FlowRate(ml/min): 30; Injections 5) provided [2,4′-bipyridin]-3-yl(4-fluoro-4-((6-(fluoromethyl)pyridin-3-yl)methyl)piperidin-1-yl)methanone (4.9 mg). Further purification by recrystallization from MeCN (1 mL) at 40° C. afforded [2,4′-bipyridin]-3-yl(4-fluoro-4-((6-(fluoromethyl)pyridin-3-yl)methyl)piperidin-1-yl)methanone (2.7 mg, 4.3%).

¹H NMR (400 MHz, CDCl₃) δ_{H} 8.65-8.82 (m, 3H), 8.22-8.36 (m, 1H), 7.77 (s, 2H), 7.52-7.64 (m, 1H), 7.37-7.48 (m, 3H), 5.31-5.58 (m, 2H), 4.47-4.75 (m, 1H), 2.42-3.09 (m, 5H), 1.65-1.90 (m, 2H), 0.84-1.54 (m, 2H). ¹⁹F NMR (376.5 MHz, CDCl₃) δ_{F} −164.63, −220.39. LC-MS purity >94%, calcd. for C₂₃H₂₂F₂N₄O [M+H]⁺ 409.1, found 409.1.

### Example 58. Synthesis of [2,4′-bipyridin]-3-yl(4-((6-(difluoromethyl)pyridin-3-yl) methyl)-4-fluoropiperidin-1-yl)methanone (Cmpd 55)

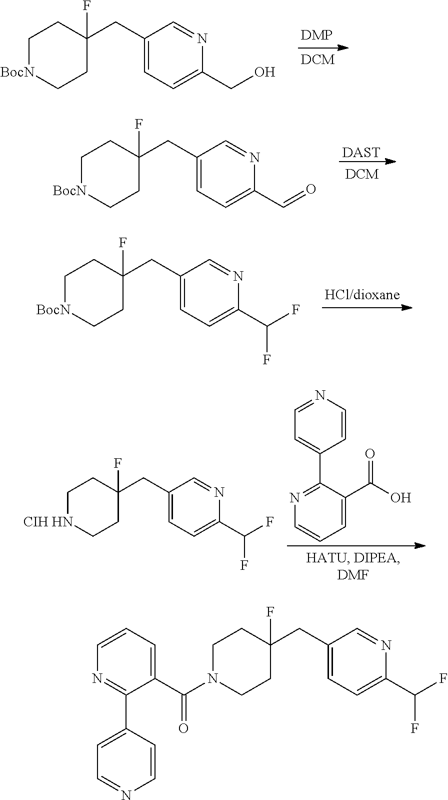

Step 1

To a solution of tert-butyl 4-fluoro-4-((6-(hydroxymethyl)pyridin-3-yl) methyl)piperidine-1-carboxylate (230 mg, 0.7090 mmol) in DCM (5 mL) was added DMP (598 mg, 1.41 mmol). After stirring at 0° C. for 0.5 h, the mixture was poured into NaHCO₃ (10 mL) and Na₂SO₃ (10 mL), and stirred for an additional 20 min. The aqueous phase was extracted with DCM (3×10 mL). The combined organic phase was washed with brine (2×10 mL), dried over anhydrous Na₂SO₄, filtered, and concentrated to give tert-butyl 4-fluoro-4-((6-formylpyridin-3-yl)methyl)piperidine-1-carboxylate (220 mg).

¹H NMR (400 MHz, CDCl₃) δ_{H} 10.08 (s, 1H), 8.62 (s, 1H), 7.93 (d, J=8.0 Hz, 1H), 7.73-7.77 (m, 1H), 3.94 (s, 4H), 3.02 (s, 2H), 1.70 (d, J=9.2 Hz, 4H), 1.45 (s, 9H)

Step 2

To a mixture of tert-butyl 4-fluoro-4-((6-formylpyridin-3-yl)methyl)piperidine-1-carboxylate (220 mg, 0.6824 mmol) in DCM (5 mL) was added DAST (549 mg, 3.41 mmol) at 0° C. and the mixture was stirred at 0° C. for 10 min. The mixture was poured into ice-water (5 mL) and NaHCO₃ (10 mL) and stirred for 10 min. The aqueous phase was extracted with DCM (2×10 mL). The combined organic phase was washed with brine (2×20 mL), dried over anhydrous Na₂SO₄, filtered, and concentrated. Purification by flash column (0-35% of EtOAc in PE) provided tert-butyl 4-((6-(difluoromethyl)pyridin-3-yl)methyl)-4-fluoropiperidine-1-carboxylate (100 mg, 42.7%).

¹H NMR (400 MHz, CDCl₃) δ_{H} 8.48 (s, 1H), 7.72 (d, J=8.0 z, 1H), 7.60 (d, J=8.0 Hz, 1H), 6.45-6.81 (m, 1H), 3.95 (s, 2H), 3.01-3.36 (m, 2H), 2.92-2.99 (m, 2H), 1.53-1.63 (m, 4H), 1.45 (s, 9H)

Step 3

To a solution of tert-butyl 4-((6-(difluoromethyl)pyridin-3-yl)methyl)-4-fluoropiperidine-1-carboxylate (100 mg, 0.2903 mmol) was added HCl/dioxane (10 mL) at 25° C. under N₂ and the reaction mixture was stirred for 2 h. The reaction mixture was concentrated to give 2-(difluoromethyl)-5-((4-fluoropiperidin-4-yl)methyl)pyridine hydrochloride (90 mg), which was used directly in the next step.

Step 4

To a solution of [2,4′-bipyridine]-3-carboxylic acid (36.4 mg, 0.182 mmol) and HATU (86.6 mg, 0.228 mmol) in DMF (2 mL) was added DIPEA (0.591 mL, 3.39 mmol). 2-(difluoromethyl)-5-((4-fluoropiperidin-4-yl)methyl)pyridine hydrochloride (40 mg, 0.1522 mmol) in DMF (2 mL) was added slowly. The mixture was stirred at 20° C. for 2 h. The reaction mixture was poured into H₂O (10 mL) and stirred for 20 min. The aqueous phase was extracted with EtOAc (3×10 mL). The combined organic phase was washed with brine (2×10 mL), dried over anhydrous Na₂SO₄, filtered, and concentrated. Purification by HPLC (Column: Phenomenex Gemini-NX 80*40 mm*3 um; Condition: water (NH₃H₂O)-ACN; Begin B: 21; End B: 51; Gradient Time(min): 8; 100% B Hold Time(min): 2; FlowRate(ml/min): 30; Injections 6) provided [2,4′-bipyridin]-3-yl(4-((6-(difluoromethyl)pyridin-3-yl)methyl)-4-fluoropiperidin-1-yl)methanone (33.2 mg, 24%).

¹H NMR (400 MHz, CDCl₃) δ_{H} 8.66-8.84 (m, 3H), 8.31-8.45 (m, 1H), 7.72-7.82 (m, 2H), 7.48-7.69 (m, 2H), 7.43 (m 1H), 7.40-7.48 (m, 1H), 6.40-6.81 (m, 1H), 4.51-4.75 (m, 1H), 2.82-3.11 (m, 3H), 2.45-2.78 (m, 2H), 2.37-2.78 (m, 1H), 1.63-1.82 (m, 1H), 1.05-1.52 (m, 2H). F NMR (376.5 MHz, CDCl₃) δ_{F} −115.63, −164.69. LCMS purity >96%, calcd. for C₂₃H₂₁F₃N₄O [M+H]⁺ 427.2, found 427.2.

### Example 59. Synthesis of (4-fluoro-4-((5-(trifluoromethyl)pyrimidin-2-yl)methyl)piperidin-1-yl)(2-(pyrimidin-4-yl)pyridin-3-yl)methanone (Cmpd 56)

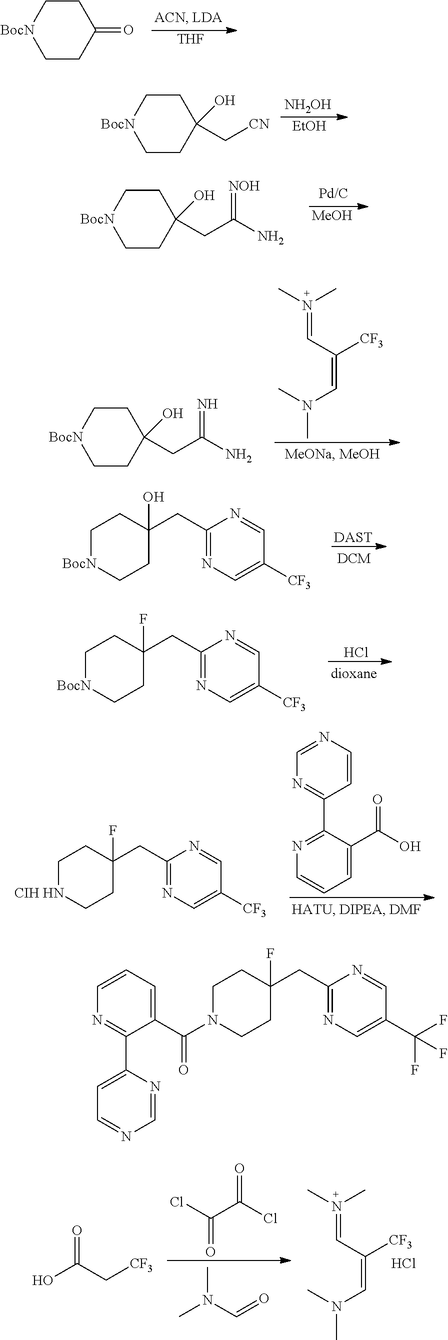

Step 1

LDA (50 mL, 100.0 mmol, 2M) was added to THE (150 mL) and acetonitrile (4.10 g, 100.0 mmol) was added into the mixture at −78° C. and stirred for 1 h. Then tert-butyl 4-oxopiperidine-1-carboxylate (10.0 g, 50.1 mmol) in THE (50 mL) was added to the reaction system slowly over 30 min at −78° C. The reaction mixture was stirred at −78° C. for 4 h. The reaction was slowly raised to room temperature and maintained for 1 h. It was quenched with saturated ammonium chloride aqueous solution, water (150 mL) was added, and extraction was performed with ethyl acetate (600 mL). The organic phases were combined, washed with saturated brine (500 mL), and dried over anhydrous sodium sulfate, filtered, and concentrated. Purification by flash column (0˜40% of EtOAc in PE) provided tert-butyl 4-(cyanomethyl)-4-hydroxypiperidine-1-carboxylate (4.2 g, 35%).

¹H NMR (400 MHz, CDCl₃) δ_{H} 4.00-3.85 (m, 2H), 3.25-3.10 (m, 2H), 2.50 (s, 2H), 1.80-1.55 (m, 4H), 1.45 (s, 9H).

Step 2

To a solution of tert-butyl 4-(cyanomethyl)-4-hydroxypiperidine-1-carboxylate (3 g, 12.4 mmol) in EtOH (30 mL) was added NH₄₀H (20 mL) in one portion at 25° C. The mixture was stirred at 70° C. for 2 h. The mixture was extracted with EtOAc (50 mL), dried over Na₂SO₄, filtered, and concentrated to give the tert-butyl (E)-4-(2-amino-2-(hydroxyimino)ethyl)-4-hydroxypiperidine-1-carboxylate (2.2 g).

¹H NMR (400 MHz, DMSO-d₆) δ_{H} 8.85-8.90 (m, 1H), 5.34 (s, 2H), 4.71 (s, 1H), 3.60-3.50 (m, 2H), 3.18-3.10 (m, 2H), 2.10 (s, 2H), 1.50-1.40 (m, 4H), 1.38 (s, 9H).

Step 3

To a solution of tert-butyl (E)-4-(2-amino-2-(hydroxyimino)ethyl)-4-hydroxypiperidine-1-carboxylate (2 g, 7.31 mmol) in MeOH (20 mL) was added Pd/C (0.2 g, 10% wet) under N₂. The mixture was stirred under H₂ (15 psi) at 40° C. for 48 h. The mixture was filtered and concentrated to give tert-butyl 4-(2-amino-2-iminoethyl)-4-hydroxypiperidine-1-carboxylate (1.2 g).

¹H NMR (400 MHz, DMSO-d₆) δ_{H} 5.34 (s, 2H), 5.00-4.50 (m, 2H), 3.60-3.50 (m, 2H), 3.08 (s, 2H), 2.10 (s, 2H), 1.50-1.40 (m, 4H), 1.46 (s, 9H).

Step 4

Oxalic dichloride (10.3 g, 81.9 mmol) was added into DMF (15 mL) slowly over 0.5 h at 0° C. A white solid formed after 0.1 h and then 3,3,3-trifluoropropanoic acid (5 g, 39.0 mmol) was slowly added at 0° C. After 10 minutes, the reaction was heated at 70° C. for 1 h. The reaction mixture was cooled and concentrated under reduced pressure to give (E)-N-(3-(dimethylamino)-2-(trifluoromethyl)allylidene)-N-methylmethanaminium hydrochloride (8 g) which was carried directly to the next step.

¹H NMR (400 MHz, CDCl₃) δ_{H} 8.91 (s, 2H), 3.57 (s, 6H), 3.26 (d, J=2.4 Hz, 6H).

Step 5

To a solution of tert-butyl 4-(2-amino-2-iminoethyl)-4-hydroxypiperidine-1-carboxylate (1.2 g, 4.66 mmol) in MeOH (30 mL) was added MeONa (1.25 g, 23.3 mmol) and (E)-N-(3-(dimethylamino)-2-(trifluoromethyl)allylidene)-N-methylmethanaminium hydrochloride (1.36 g, 6.98 mmol) in one portion at 10° C. under N₂. The mixture was stirred at 10° C. for 2 h. The mixture was diluted with H₂O (50 mL). The aqueous phase was extracted with EtOAc (3×30 mL). The combined organic phase was washed with saturated brine (2×20 mL), dried over anhydrous Na₂SO₄, filtered and concentrated. Purification by flash column (0-30% of EtOAc in PE) provided tert-butyl 4-hydroxy-4-((5-(trifluoromethyl)pyrimidin-2-yl)methyl)piperidine-1-carboxylate (200 mg).

¹H NMR (400 MHz, CDCl₃) δ_{H} 8.94 (s, 2H), 3.81 (m, 2H), 3.29-3.20 (m, 5H), 1.57 (dd, J=4.0, 7.2 Hz, 4H), 1.45 (s, 9H). ¹⁹F NMR (376 MHz, CDCl₃) δ_{F} −62.400.

Step 6

To a mixture of tert-butyl 4-hydroxy-4-((5-(trifluoromethyl)pyrimidin-2-yl)methyl)piperidine-1-carboxylate (150 mg, 0.975 mmol) in DCM (10 mL) was added DAST (535 mg, 3.32 mmol) at 0° C. and the mixture was stirred at 0° C. for 30 min. The residue was poured into ice-water (10 mL) and NaHCO₃ (20 mL) and stirred for 10 min. The aqueous phase was extracted with DCM (2×20 mL). The combined organic phase was washed with saturated brine (2×20 mL), dried over anhydrous Na₂SO₄, filtered and concentrated. Purification by flash column (0-30% EtOAc in PE) and further purification by SFC (Column: DAICEL CHIRALPAK IC (250 mm*30 mm, 10 um); Condition: 0.1% NH₃H₂O ETOH; Begin B: 10%; End B: 10%; Gradient Time(min); 100% B Hold Time(min); Flow Rate (ml/min): 50; Injections: 60) provided tert-butyl 4-fluoro-4-((5-(trifluoromethyl)pyrimidin-2-yl)methyl)piperidine-1-carboxylate (25 mg, 17%).

¹H NMR (400 MHz, CDCl₃) δ_{H} 8.95 (s, 2H), 4.00-3.88 (m, 2H), 3.40 (d, J=18.4 Hz, 2H), 3.16-3.01 (m, 2H), 1.95-1.86 (m, 2H), 1.85-1.67 (m, 2H), 1.45 (s, 9H). ¹⁹F NMR (376 MHz, CDCl₃). δ_{F} −62.3 90.

Step 7

To a solution of tert-butyl 4-fluoro-4-((5-(trifluoromethyl)pyrimidin-2-yl)methyl)piperidine-1-carboxylate (25 mg, 0.0688 mmol) in 1,4-dioxane (5 mL) was added hydrogen chloride (5 mL, 20.0 mmol, 4 M in 1,4-dioxane) at 20° C. under N₂ and the reaction mixture was stirred at 20° C. for 2 h. The reaction mixture was concentrated under reduced pressure to give 2-((4-fluoropiperidin-4-yl)methyl)-5-(trifluoromethyl)pyrimidine hydrochloride (30 mg).

Step 8

To a solution of 2-(pyrimidin-4-yl)pyridine-3-carboxylic acid (24.1 mg, 0.12 mmol) and HATU (57.0 mg, 0.15 mmol) in DMF (2 mL) was added DIPEA (0.0872 mL, 0.5 mmol, 0.74 g/mL) at 20° C. and stirred for 15 min. 2-((4-fluoropiperidin-4-yl)methyl)-5-(trifluoromethyl)pyrimidine hydrochloride (30 mg, 0.1 mmol) in DMF (2 mL) was added slowly at 20° C. The mixture was stirred at 20° C. for 16 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 22; End B: 52; Gradient Time(min): 8; 100% B Hold Time(min): 3; Flow Rate (ml/min): 30; Injections: 5) gave (4-fluoro-4-((5-(trifluoromethyl)pyrimidin-2-yl)methyl)piperidin-1-yl)(2-(pyrimidin-4-yl)pyridin-3-yl)methanone (13.2 mg, 30%).

¹H NMR (400 MHz, CDCl₃) δ_{H} 9.20-9.01 (m, 1H), 8.96 (s, 2H), 8.88 (d, J=4.8 Hz, 1H), 8.75 (m, 1H), 8.30-8.22 (m, 1H), 7.74-7.64 (m, 1H), 7.46 (m, 1H), 4.74-4.55 (m, 1H), 3.43 (d, J=18.4 Hz, 3H), 3.30-3.11 (m, 2H), 2.12 (s, 2H), 1.92-1.69 (m, 2H). ¹⁹F NMR (376 MHz, CDCl₃). δ_{F} −62.381, −159.897. LCMS purity 99%, MS ESI calcd. For C₂₁H₁₈F₄N₆O [M+H]⁺ 447.1, found 447.1.

### Example 60. Synthesis of (4-((5-chloropyrazin-2-yl)methyl)-4-fluoropiperidin-1-yl)(2-(pyrimidin-4-yl)pyridin-3-yl)methanone (Cmpd 95)

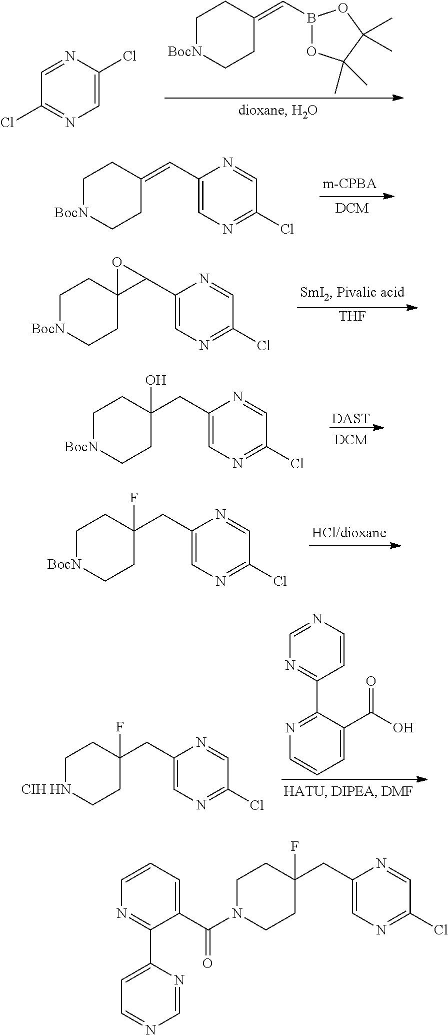

Step 1

A mixture of Pd(dppf)Cl₂ (245 mg, 0.335 mmol), Na₂CO₃ (1.42 g, 13.4 mmol), 2,5-dichloropyrazine (1 g, 6.71 mmol) and tert-butyl 4-[(4,4,5,5-tetramethyl-1,3,2-dioxaborolan-2-yl) methylidene]piperidine-1-carboxylate (1.73 g, 5.36 mmol) in dioxane (20 mL) and water (4 mL) was stirred at 100° C. for 16 h under N₂. After cooling to 20° C., the mixture was poured into water (50 mL) and extracted with EtOAc (50 mL×2). The combined organic phase was washed with water (50 mL) and brine (100 mL), dried over Na₂SO₄, filtered and concentrated. Purification by flash column (0%˜20% EtOAc in PE) provided tert-butyl 4-((5-chloropyrazin-2-yl)methylene)piperidine-1-carboxylate (1 g).

¹H NMR (400 MHz, CDCl₃) δ_{H} 8.44 (m, 1H), 8.17 (m, 1H), 6.59 (s, 1H), 3.57 (m, 2H), 3.49 (m, 2H), 2.88-2.80 (m, 2H), 2.44 (t, J=5.2 Hz, 2H), 1.48 (s, 9H).

Step 2

To a solution of tert-butyl 4-((5-chloropyrazin-2-yl)methylene)piperidine-1-carboxylate (1.2 g, 3.87 mmol) in DCM (50 mL) was added m-CPBA (3.91 g, 19.3 mmol, 85%) at 0° C. and the reaction mixture was stirred at 0° C. for 5 h. The reaction mixture was diluted with Na₂S₂O₃ (100 mL) and stirred 10 min, then extracted with DCM (50 ml×2) and the combined organic phase was dried over Na₂SO₄, filtered, and concentrated. Purification by flash column (0-20% EtOA in PE) provided tert-butyl 2-(5-chloropyrazin-2-yl)-1-oxa-6-azaspiro[2.5]octane-6-carboxylate (1 Ig).

¹H NMR (400 MHz, CDCl₃) δ_{H} 8.55 (m, 1H), 8.34 (m, 1H), 4.19 (s, 1H), 3.82-3.72 (m, 1H), 3.62 (m, 1H), 3.51-3.44 (m, 2H), 2.00 (m, 1H), 1.78 (m, 1H), 1.52-1.48 (m, 2H), 1.46 (s, 9H).

Step 3

To a solution of tert-butyl 2-(5-chloropyrazin-2-yl)-1-oxa-6-azaspiro[2.5]octane-6-carboxylate (200 mg, 0.613 mmol) in HMPA (6 mL) at 20° C. was added SmI₂ (18.3 mL, 0.1M in THF, 1.83 mmol). A solution of pivalic acid (5.40 mL, 0.17 M in THF, 0.919 mmol) was added and the solution was stirred for 48 h. The reaction was quenched with an aqueous solution of sodium potassium tartrate (40 mL). The mixture was extracted with EtOAc (3×20 mL) and the organic layer was washed with H₂O (2×20 mL), dried with Na₂SO₄, and filtered. The solvent was removed in vacuo and the crude product was purified by flash column (0-40% EtOAc in PE) to afford tert-butyl 4-((5-chloropyrazin-2-yl)methyl)-4-hydroxypiperidine-1-carboxylate (120 mg).

¹H NMR (400 MHz, CDCl₃) δ_{H} 8.56 (m, 1H), 8.34 (m, 1H), 3.89-3.72 (m, 2H), 3.50-3.45 (m, 2H), 2.00 (m, 1H), 1.78 (m, 1H), 1.58-1.55 (m, 2H), 1.46 (s, 9H).

Step 4

To a mixture of tert-butyl 4-((5-chloropyrazin-2-yl)methyl)-4-hydroxypiperidine-1-carboxylate (120 mg, 0.366 mmol) in DCM (10 mL) was added DAST (117 mg, 0.732 mmol) at 0° C. and the mixture was stirred at 0° C. for 30 min. The residue was poured into ice-water (10 mL) and NaHCO₃ (10 mL) and stirred for 10 min. The aqueous phase was extracted with DCM (2×20 mL). The combined organic phase was washed with saturated brine (2×20 mL), dried over anhydrous Na₂SO₄, filtered, and concentrated. Purification by flash column (0-50% EtOAc in PE) and further purification by SFC (Column: DAICEL CHIRALCEL OD-H (250 mm*30 mm, 5 um); Condition: 0.1% NH₃H₂O ETOH; Begin B: 15%; End B; 15%; Flow Rate (ml/min): 60; Injections: 50) provided tert-butyl 4-((5-chloropyrazin-2-yl)methyl)-4-fluoropiperidine-1-carboxylate (10 mg, 8%).

¹H NMR (400 MHz, CDCl₃) δ_{H} 8.48 (m, 1H), 8.28 (m, 1H), 3.96 (m, 2H), 3.05 (t, J=12.0 Hz, 2H), 1.89-1.73 (m, 4H), 1.45 (s, 9H). ¹⁹F NMR (376.5 MHz, CDCl₃) δ_{F} −159.686.

Step 5

To a solution of tert-butyl 4-((5-chloropyrazin-2-yl)methyl)-4-fluoropiperidine-1-carboxylate (20 mg, 0.0606 mmol) in 1,4-dioxane (5 mL) was added hydrogen chloride (5 mL, 20.0 mmol, 4 M in 1,4-dioxane) at 20° C. under N₂ and the reaction mixture was stirred at 20° C. for 2 h. The reaction mixture was concentrated under reduced pressure to give 2-chloro-5-((4-fluoropiperidin-4-yl)methyl)pyrazine hydrochloride (20 mg), which was carried directly into the next step.

Step 6

To a solution of 2-(pyrimidin-4-yl)pyridine-3-carboxylic acid (18.1 mg, 0.09 mmol) and HATU (42.5 mg, 0.11 mmol) in DMF (2 mL) was added DIPEA (0.0654 mL, 0.375 mmol, 0) at 20° C. and stirred for 15 min. 2-chloro-5-((4-fluoropiperidin-4-yl)methyl)pyrazine hydrochloride (20 mg, 0.0751 mmol) in DMF (2 mL) was added slowly at 20° C. and stirred for 1 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 25; End B: 55; Gradient Time(min): 8; 100% B Hold Time(min): 2; Flow Rate (ml/min): 30; Injections 4) provided (4-((5-chloropyrazin-2-yl)methyl)-4-fluoropiperidin-1-yl)(2-(pyrimidin-4-yl)pyridin-3-yl)methanone (6.9 mg, 22%).

¹H NMR (400 MHz, CDCl₃) δ_{H} 9.21-8.83 (m, 2H), 8.75 (d, J=2.4 Hz, 1H), 8.50 (s, 1H), 8.34-8.23 (m, 2H), 7.73-7.64 (m, 1H), 7.49-7.43 (m, 1H), 4.78-4.56 (m, 1H), 3.48-3.36 (m, 3H), 3.29-3.10 (m, 2H), 2.20-1.97 (m, 2H), 1.96-1.70 (m, 2H). ¹⁹F NMR (376.5 MHz, CDCl₃). S_{F}-159.078. LCMS purity 99%, MS ESI calcd. For C₂₀H₁₈ClFN₆O [M+Na]⁺ 435.1, found 435.1.

### Example 61. Synthesis of [2,4′-bipyridin]-3-yl(4-((5-chloropyrazin-2-yl)methyl)-4-fluoropiperidin-1-yl)methanone (Cmpd 96)

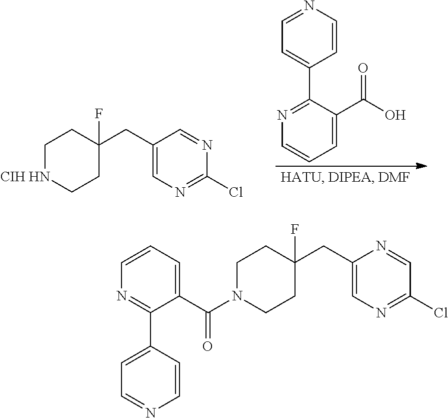

To a solution of [2, 4′-bipyridine]-3-carboxylic acid (9.00 mg, 0.045 mmol) and HATU (21.3 mg, 0.0562 mmol) in DMF (2 mL) was added DIPEA (0.0325 mL, 0.187 mmol). 2-chloro-5-((4-fluoropiperidin-4-yl)methyl)pyrazine hydrochloride (10 mg, 0.0375 mmol) in DMF (2 mL) was added slowly. The mixture was stirred at 20° C. for 1 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, concentrated, and purified by HPLC (Column: Phenomenex C18 80*40 mm*3 um; Condition: water (NH₃H₂O)-ACN; Begin B: 18; End B: 48; Gradient Time(min): 8; 100% B Hold Time(min): 2; Flow Rate (ml/min): 30; Injections: 3) to give [2,4′-bipyridin]-3-yl(4-((5-chloropyrazin-2-yl)methyl)-4-fluoropiperidin-1-yl)methanone (3 mg, 19%).

¹H NMR (400 MHz, CDCl₃) δ_{H} 8.80 (d, J=4.8 Hz, 1H), 8.68 (s, 2H), 8.47 (s, 1H), 8.31 (s, 1H), 7.77 (m, 3H), 7.45 (dd, J=4.8, 7.6 Hz, 1H), 4.68-4.58 (m, 1H), 3.42-3.13 (m, 2H), 3.12-2.86 (m, 2H), 2.06-1.82 (m, 2H), 1.73-1.58 (m, 1H). ¹⁹F NMR (376.5 MHz, CDCl₃). δ_{F} −159.990. LCMS purity 99%, MS ESI calcd. For C₂₁H₁₉ClFN₅O [M+H]⁺ 412.1, found 412.1.

### Example 62 and Example 63. Synthesis of (R)-(4-fluoro-4-(1-(6-(trifluoromethyl)pyridin-3-yl)vinyl)piperidin-1-yl)(2-(pyrimidin-4-yl)pyridin-3-yl)methanone (Cmpd 62) and Synthesis of (S)-(4-fluoro-4-(1-(6-(trifluoromethyl)pyridin-3-yl)ethyl)piperidin-1-yl)(2-(pyrimidin-4-yl)pyridin-3-yl)methanone (Cmpd 61)

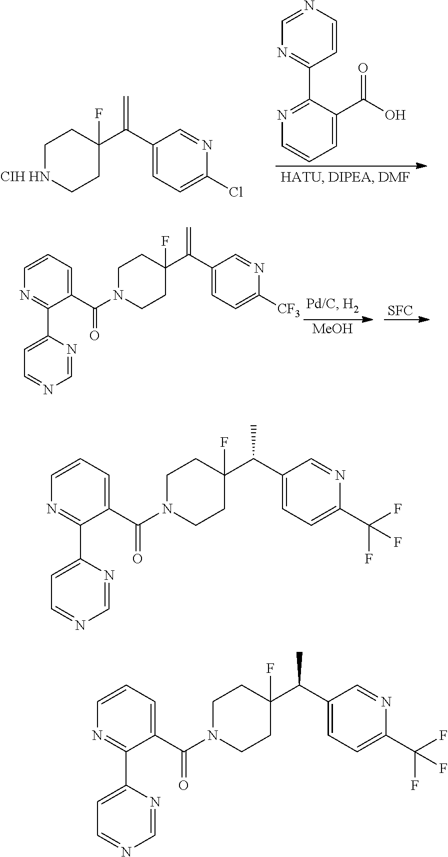

5-(1-(4-fluoropiperidin-4-yl)vinyl)-2-(trifluoromethyl)pyridine hydrochloride was prepared according to the procedures detailed in Examples 69 and 70 of this disclosure.

To a solution of 2-(pyrimidin-4-yl) pyridine-3-carboxylic acid (186 mg, 926 μmol) and HATU (437 mg, 1.15 mmol) in DMF (2 mL) was added DIPEA (0.672 mL, 3.86 mmol). 5-(1-(4-fluoropiperidin-4-yl)vinyl)-2-(trifluoromethyl)pyridine hydrochloride (240 mg, 0.77 mmol) in DMF (3 mL) was added slowly. The mixture was stirred at 20° C. for 2 h. The reaction mixture poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated to give (4-fluoro-4-(1-(6-(trifluoromethyl)pyridin-3-yl)vinyl)piperidin-1-yl)(2-(pyrimidin-4-yl)pyridin-3-yl)methanone (300 mg).

To a solution of (4-fluoro-4-(1-(6-(trifluoromethyl)pyridin-3-yl)vinyl)piperidin-1-yl)(2-(pyrimidin-4-yl)pyridin-3-yl)methanone (300 mg) in THE (5 mL) was added Pd/C (wet, 50 mg) at 25° C. Then the solution was hydrogenated under 15 psi of hydrogen at 25° C. for 16 hours. The mixture was filtered through a pad of celite and washed with THE (3×5 mL) and the filtrate was concentrated in vacuum. The product was purified by flash column (0-30% of EtOAc in PE) and further purified by HPLC (Column: Phenomenex Gemini-C18 80*40 mm*3 um; Condition: water (NH₃H₂O)-ACN; Begin B: 34; End B: 64; Gradient Time(min): 8; 100% B Hold Time(min): 2.3; Flow Rate (ml/min): 30; Injections: 5) to give (4-fluoro-4-(1-(6-(trifluoromethyl)pyridin-3-yl)ethyl)piperidin-1-yl)(2-(pyrimidin-4-yl)pyridin-3-yl)methanone (72 mg, 24%).

(4-fluoro-4-(1-(6-(trifluoromethyl)pyridin-3-yl)ethyl)piperidin-1-yl)(2-(pyrimidin-4-yl)pyridin-3-yl)methanone (72 mg, 0.1567 mmol) was separated into its enantiomers by SFC (Column: DAICEL CHIRALPAK AD (250 mm*30 mm, 10 um); Condition: 0.1% NH₃H₂O ETOH; Begin B: 25%; End B: 25%; Flow Rate (ml/min): 60; Injections: 60) to afford (R)-(4-fluoro-4-(1-(6-(trifluoromethyl)pyridin-3-yl)ethyl)piperidin-1-yl)(2-(pyrimidin-4-yl)pyridin-3-yl)methanone (30 mg) and (S)-(4-fluoro-4-(1-(6-(trifluoromethyl)pyridin-3-yl)ethyl)piperidin-1-yl)(2-(pyrimidin-4-yl)pyridin-3-yl)methanone (18.4 mg, 25%). The (R)-(4-fluoro-4-(1-(6-(trifluoromethyl)pyridin-3-yl)ethyl)piperidin-1-yl)(2-(pyrimidin-4-yl)pyridin-3-yl)methanone (30 mg) was further purified by SFC (Column: (s, s) WHELK-01 (250 mm*30 mm, Sum); Condition: 0.1% NH₃H₂O ETOH; Begin B: 40%; End B: 40%; Flow Rate (ml/min): 80; Injections: 45) to afford (R)-(4-fluoro-4-(1-(6-(trifluoromethyl)pyridin-3-yl)ethyl)piperidin-1-yl)(2-(pyrimidin-4-yl)pyridin-3-yl)methanone (13.9 mg, 19%).

Cmpd 62. (R)-(4-fluoro-4-(1-(6-(trifluoromethyl)pyridin-3-yl)ethyl)piperidin-1-yl)(2-(pyrimidin-4-yl)pyridin-3-yl)methanone: ¹H NMR (400 MHz, CDCl₃) δ_{H} 9.21-9.02 (m, 1H), 8.91-8.86 (m, 1H), 8.79-8.68 (m, 1H), 8.64-8.49 (m, 1H), 8.26 (s, 1H), 7.82-7.59 (m, 3H), 7.49-7.38 (m, 1H), 4.84-4.53 (m, 1H), 3.53-2.85 (m, 4H), 2.30-1.67 (m, 3H), 1.48-1.21 (m, 4H). ¹⁹F NMR (376.5 MHz, CDCl₃) δ_{F} −67.823. MS ESI calcd. for C₂₃H₂₁F₄N₅O [M+H]⁺ 460.2, found 460.2.

Cmpd 61. (S)-(4-fluoro-4-(1-(6-(trifluoromethyl)pyridin-3-yl)ethyl)piperidin-1-yl)(2-(pyrimidin-4-yl)pyridin-3-yl)methanone: ¹H NMR (400 MHz, CDCl₃) δ_{H} 9.25-8.99 (m, 1H), 8.90-8.86 (m, 1H), 8.79-8.69 (m, 1H), 8.63-8.48 (m, 1H), 8.26 (s, 1H), 7.83-7.59 (m, 3H), 7.51-7.40 (m, 1H), 4.87-4.51 (m, 1H), 3.58-2.85 (m, 4H), 2.34-1.66 (m, 3H), 1.52-1.20 (m, 4H). ¹⁹F NMR (376.5 MHz, CDCl₃) δ_{F} −67.818. LCMS purity >95%, MS ESI calcd. for C₂₃H₂₁F₄N₅O [M+H]⁺ 460.2, found 460.2.

### Example 64. Synthesis of [2,4′-bipyridin]-3-yl(4-fluoro-4-(imidazo[1,2-a]pyridin-7-ylmethyl)piperidin-1-yl)methanone (Cmpd 63)

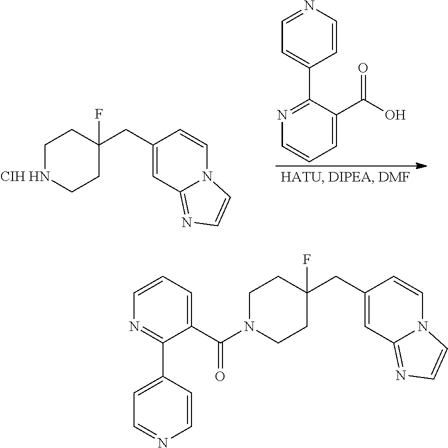

To a solution of [2,4′-bipyridine]-3-carboxylic acid (1.07 g, 5.32 mmol) and HATU (2.53 g, 6.66 mmol) in DMF (30 mL) was added DIPEA (2.316 mL, 13.32 mmol) and the mixture was stirred 10-15 min at 20° C. 7-((4-fluoropiperidin-4-yl)methyl)imidazo[1,2-a]pyridine hydrochloride (1.2 g, 4.44 mmol) in DMF (20 mL) was added slowly. The mixture was stirred at 20° C. for 1-2 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by flash Prep-HPLC (Column: Phenomenex Gemini-NX 80*40 mm*3 um; Condition: water (0.05% NH₃H₂O)-ACN; Begin B: 19; End B: 49; Gradient Time(min): 8; 100% B Hold Time(min): 3; FlowRate(ml/min): 30; Injections: 5) to give [2,4′-bipyridin]-3-yl(4-fluoro-4-(imidazo[1,2-a]pyridin-7-ylmethyl)piperidin-1-yl)methanone (89.4 mg). Further purification by SFC (Column: DAICEL CHIRALPAK AS (250 mm*30 mm, 10 um); Condition: 0.1% NH₃H₂O ETOH; Begin B: 30; End B: 30; FlowRate(ml/min): 70; Injections: 50) provided [2,4′-bipyridin]-3-yl(4-fluoro-4-(imidazo[1,2-a]pyridin-7-ylmethyl)piperidin-1-yl)methanone (7.8 mg, 15%).

¹H NMR (400 MHz, CDCl₃)^{6H}=8.78 (m, 1H), 8.70-8.69 (d, J=4.0 Hz, 2H), 8.03-8.01 (d, J=8.0 Hz, 1H), 7.82-7.70 (m, 2H), 7.65-7.52 (m, 3H), 7.42 (m, 1H), 7.29 (s, 1H), 6.73-6.46 (m, 1H), 4.72-4.33 (m, 2H), 3.16-2.46 (m, 5H), 1.48-1.15 (m, 2H), 0.14-0.17 (m, 1H). ¹⁹F NMR (376.5 MHz, CDCl₃) δ_{F} −163.19. LCMS purity 98.1%, MS ESI calcd. for C₂₄H₂₂FN₅O [M+H]⁺ 415.9, found 415.9.

### Example 65. Synthesis of (4-((6-(difluoromethyl)pyridin-3-yl)methyl)-4-fluoropiperidin-1-yl)(2-(pyrimidin-4-yl)pyridin-3-yl)methanone (Cmpd 64)

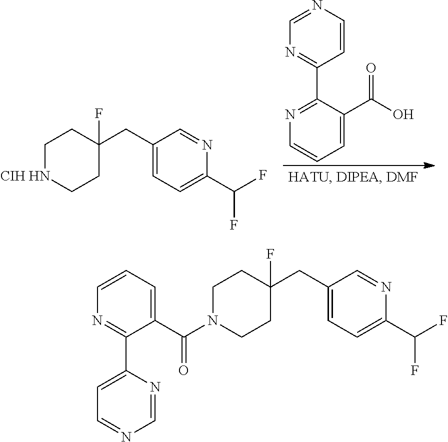

To a solution of 2-(pyrimidin-4-yl)pyridine-3-carboxylic acid (29.9 mg, 0.149 mmol) and HATU (70.7 mg, 0.186 mmol) in DMF (2 mL) was added DIPEA (0.108 mL, 0.623 mmol). 2-(difluoromethyl)-5-((4-fluoropiperidin-4-yl)methyl)pyridine hydrochloride (35 mg, 0.12 mmol) in DMF (2 mL) was added slowly. The mixture was stirred at 20° C. for 2 h. The reaction mixture poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 30; End B: 60; Gradient Time (min): 8; 100% B Hold Time (min): 2; Flow Rate (ml/min): 30; Injections: 4) provided (4-((6-(difluoromethyl)pyridin-3-yl)methyl)-4-fluoropiperidin-1-yl)(2-(pyrimidin-4-yl)pyridin-3-yl) methanone (7.2 mg, 13%).

¹H NMR (400 MHz, CDCl₃) δ_{H} 9.23-8.96 (m, 1H), 8.89-8.87 (m, 1H), 8.76-8.75 (m, 1H), 8.48 (s, 1H), 8.27 (s, 1H), 7.76-7.64 (m, 2H), 7.61-7.59 (m, 1H), 7.48-7.44 (m, 1H), 6.85-6.45 (m, 1H), 4.75-4.56 (m, 1H), 3.50-3.30 (m, 1H), 3.24-2.86 (m, 4H), 2.07-1.69 (m, 4H). ¹⁹F NMR (376.5 MHz, CDCl₃) δ_{F} −115.678, −162.562, −162.817. LCMS purity >99%, calcd. for C₂₂H₂₀F₃N₅ONa [M+Na]⁺ 450.3, found 450.3.

### Example 66. Synthesis of (4-((6-chloropyridazin-3-yl)methyl)-4-fluoropiperidin-1-yl)(2-(pyrimidin-4-yl)pyridin-3-yl)methanone (Cmpd 65)

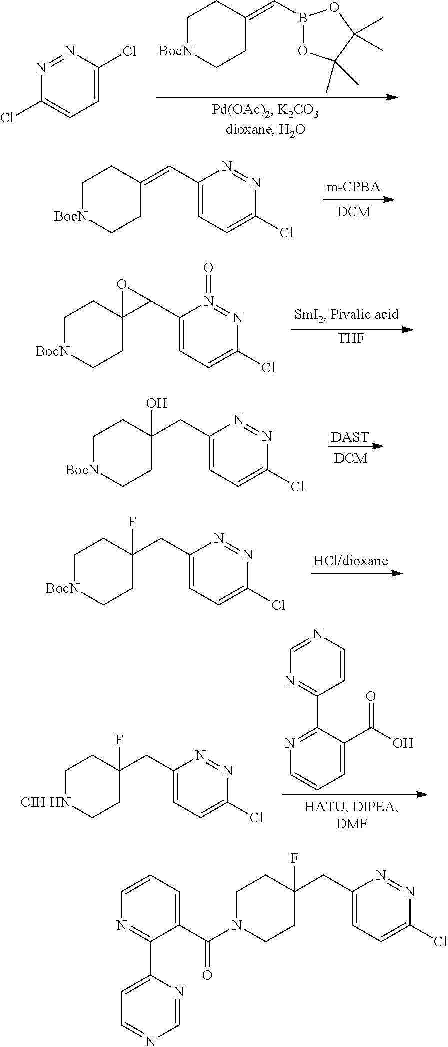

Step 1

A mixture of Pd(dppf)Cl₂ (245 mg, 0.335 mmol), Na₂CO₃ (1.42 g, 13.4 mmol), 3,6-dichloropyridazine (1 g, 6.71 mmol) and tert-butyl 4-[(4,4,5,5-tetramethyl-1,3,2-dioxaborolan-2-yl)methylidene]piperidine-1-carboxylate (1.51 g, 4.69 mmol) in dioxane (20 mL) and water (4 mL) was stirred at 20° C. Then the mixture was stirred at 100° C. for 16 hours under N₂. After cooling to 20° C., the mixture was poured into water (50 mL) and extracted with EtOAc (50 mL×2). The combined organic phase was washed with water (50 mL) and brine (100 mL), dried over Na₂SO₄, filtered, and concentrated. The crude product was purified by column (0-30% of EtOAc in PE) to give tert-butyl 4-((6-chloropyridazin-3-yl)methylene)piperidine-1-carboxylate (700 mg).

¹H NMR (400 MHz, CDCl₃) δ_{H} 7.44 (d, J=8.8 Hz, 1H), 7.28 (s, 1H), 6.39 (s, 1H), 3.60-3.54 (m, 2H), 3.49 (m, 2H), 2.86 (m, 2H), 2.42 (m, 2H), 1.48 (s, 9H).

Step 2

To a solution of tert-butyl 4-((6-chloropyridazin-3-yl)methylene)piperidine-1-carboxylate (700 mg, 2.25 mmol) in DCM (20 mL) was added m-CPBA (2.27 g, 11.2 mmol, 85% purity) at 0° C. and the reaction mixture was stirred at 0° C. for 5 h. The reaction mixture was diluted with Na₂S₂O₃ (50 mL), extracted with DCM (20 ml×3) and the combined organic phase was dried over Na₂SO₄, filtered, and concentrated. Purification by flash column (0˜40% of EtOAc in PE) gave 6-(6-(tert-butoxycarbonyl)-1-oxa-6-azaspiro[2.5]octan-2-yl)-3-chloropyridazine 1-oxide (300 mg).

¹H NMR (400 MHz, CDCl₃) δ_{H} 7.62-7.49 (m, 1H), 7.46-7.16 (m, 1H), 4.10 (s, 1H), 4.34-4.07 (m, 1H), 3.83-3.68 (m, 1H), 3.67-3.47 (m, 1H), 3.45-3.30 (m, 1H), 2.02-1.57 (m, 4H), 1.45 (s, 9H).

Step 3

To a solution of 6-(6-(tert-butoxycarbonyl)-1-oxa-6-azaspiro[2.5]octan-2-yl)-3-chloropyridazine 1-oxide (300 mg, 0.877 mmol) in HMPA (9 mL) at 20° C. was added SmI₂ (21.9 mL, 0.1M in THF, 2.19 mmol). A solution of pivalic acid (7.17 mL, 0.17 M in THF, 1.22 mmol) was added and the solution was allowed to stir for 24 h. The reaction was quenched with a solution of sodium potassium tartrate (50 mL). The mixture was extracted with EtOAc (3×20 mL) and the organic layer was washed with H₂O (2×20 mL) and dried with Na₂SO₄. The solvent was removed in vacuo and the crude product was purified by flash column (0-40% EtOAc in PE) to afford tert-butyl 4-((6-chloropyridazin-3-yl)methyl)-4-hydroxypiperidine-1-carboxylate (300 mg).

Step 4

To a mixture of tert-butyl 4-((6-chloropyridazin-3-yl)methyl)-4-hydroxypiperidine-1-carboxylate (300 mg, 0.915 mmol) in DCM (10 mL) was added DAST (293 mg, 1.82 mmol) at 0° C., the mixture was stirred at 0° C. for 20 min. The residue was poured into ice-water (10 mL) and NaHCO₃ (10 mL) and stirred for 10 min. The aqueous phase was extracted with DCM (2×20 mL). The combined organic phase was washed with saturated brine (2×20 mL), dried over anhydrous Na₂SO₄, filtered, and concentrated. Purification by flash column (0-50% EtOAc in PE) and further purified by SFC (Column: DAICEL CHIRALPAK AD (250 mm*30 mm, 10 um); Condition: 0.1% NH₃H₂O ETOH; Begin B: 20%; End B: 20%; FlowRate(ml/min): 70; Injections: 70) to give tert-butyl 4-((6-chloropyridazin-3-yl)methyl)-4-fluoropiperidine-1-carboxylate (28 mg).

¹H NMR (400 MHz, CDCl₃) δ_{H} 7.47 (s, 2H), 4.01-3.82 (m, 2H), 3.33 (s, 1H), 3.27 (s, 1H), 3.12-3.00 (m, 2H), 1.75 (m, 2H), 1.71-1.65 (m, 2H), 1.45 (s, 9H). ¹⁹F NMR (376.5 MHz, CDCl₃) δ_{F}−160.79.

Step 5

To a solution of tert-butyl 4-((6-chloropyridazin-3-yl)methyl)-4-fluoropiperidine-1-carboxylate (28 mg, 0.0848 mmol) in 1,4-dioxane (5 mL) was added hydrogen chloride (5 mL, 20.0 mmol, 4 M in 1,4-dioxane) at 20° C. under N₂ and the reaction mixture was stirred at 20° C. for 1 h. The reaction mixture was concentrated under reduced pressure to give 3-chloro-6-((4-fluoropiperidin-4-yl)methyl)pyridazine hydrochloride (30 mg).

Step 6

To a solution of 2-(pyrimidin-4-yl)pyridine-3-carboxylic acid (26.9 mg, 0.134 mmol) and HATU (63.8 mg, 0.168 mmol) in DMF (2 mL) was added DIPEA (0.0977 mL, 0.56 mmol, 0.74 g/mL) at 20° C. and stirred for 15 min. 3-chloro-6-((4-fluoropiperidin-4-yl)methyl)pyridazine hydrochloride (30 mg, 0.112 mmol) in DMF (2 mL) was added slowly at 20° C. The mixture was stirred at 20° C. for 2 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 19; End B: 49; Gradient Time(min): 8; 100% B Hold Time(min): 2; FlowRate(ml/min): 30; Injections: 4) provided (4-((6-chloropyridazin-3-yl)methyl)-4-fluoropiperidin-1-yl)(2-(pyrimidin-4-yl)pyridin-3-yl)methanone (8.4 mg, 18%).

¹H NMR (400 MHz, CDCl₃) δ_{H} 9.23-9.10 (m, 1H), 8.93-8.83 (m, 1H), 8.75 (dd, J=1.6, 4.8 Hz, 1H), 8.25 (s, 1H), 7.75-7.62 (m, 1H), 7.52-7.40 (m, 3H), 4.70-4.46 (m, 1H), 3.52-3.39 (m, 1H), 3.39-3.29 (m, 2H), 3.27-3.12 (m, 2H), 2.10-1.89 (m, 2H), 1.58 (m, 2H). ¹⁹F NMR (376.5 MHz, CDCl₃). δ_{F} −160.026, 161.316. LCMS purity 99%, MS ESI calcd. For C₂₀H₁₈ClFN₆ONa [M+Na]⁺ 435.1, found 435.1.

### Example 67. Synthesis of [2,4′-bipyridin]-3-yl(4-((6-chloropyridazin-3-yl)methyl)-4-fluoropiperidin-1-yl)methanone (Cmpd 86)

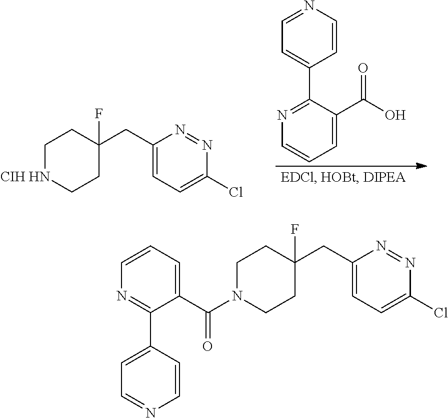

To a solution of [2,4′-bipyridine]-3-carboxylic acid (36.0 mg, 0.180 mmol), EDCl (34.5 mg, 0.180 mmol) and HOBt (24.3 mg, 0.180 mmol) in DMF (2 mL) was added DIPEA (0.0785 mL, 0.450 mmol, 0.74 g/mL) at 20° C. and the mixture was stirred for 15 min. 3-chloro-6-((4-fluoropiperidin-4-yl)methyl)pyridazine hydrochloride (40 mg, 0.150 mmol) in DMF (2 mL) was added slowly. The mixture was stirred at 20° C. for 16 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by column (0-10% MeOH in DCM) and further purification by SFC (Column: DAICEL CHIRALCEL OJ (250 mm*30 mm, 10 um); Condition: 0.1% NH₃H₂O ETOH; Begin B: 30%; End B: 30%; Gradient Time(min); 100% B Hold Time(min); Flow Rate (ml/min): 70; Injections: 35) provided [2,4′-bipyridin]-3-yl(4-((6-chloropyridazin-3-yl)methyl)-4-fluoropiperidin-1-yl)methanone (5.3 mg, 10% yield).

¹H NMR (400 MHz, CDCl₃) δ_{H} 8.79-8.77 (m, 1H), 8.76-8.66 (m, 2H), 7.82-7.75 (m, 1H), 7.72 (d, J=5.2 Hz, 1H), 7.61 (d, J=5.2 Hz, 1H), 7.47-7.37 (m, 2H), 7.31-7.26 (m, 1H), 4.54 (t, J=14.8 Hz, 1H), 3.31-3.16 (m, 1H), 3.11-2.92 (m, 3H), 2.90-2.79 (m, 1H), 2.70-2.47 (m, 1H), 1.92-1.75 (m, 1H), 1.52-1.23 (m, 2H). ¹⁹F NMR (376.5 MHz, CDCl₃). δ_{F}−160.026, −161.316. LCMS purity 96%, MS ESI calcd. For C₂₁H₁₉ClFN₅O [M+H]⁺ 412.1, found 412.1.

### Example 68. Synthesis of 1-((4-fluoro-1-(2-(pyrimidin-4-yl)nicotinoyl)piperidin-4-yl)methyl)-1H-pyrazole-4-carbonitrile (Cmpd 66)

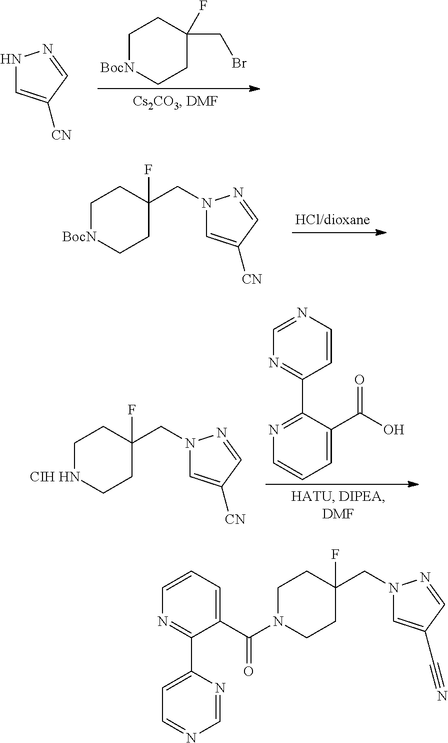

Step 1

To a solution 1H-pyrazole-4-carbonitrile (500 mg, 2.14 mmol) and Cs₂CO₃ (2.62 g, 8.05 mmol) in DMF (30 mL) was added tert-butyl 4-(bromomethyl)-4-fluoropiperidine-1-carboxylate (1.59 g, 5.37 mmol) at 80° C. under N₂ and the mixture was stirred for 48 hours. The mixture was concentrated and the residue was purified by flash column (0-40% of EtOAc in PE) to give tert-butyl 4-((4-cyano-1H-pyrazol-1-yl)methyl)-4-fluoropiperidine-1-carboxylate (800 mg, 48%).

Step 2

To a mixture of tert-butyl 4-((4-cyano-1H-pyrazol-1-yl)methyl)-4-fluoropiperidine-1-carboxylate (0.8 g, 2.59 mmol) in dioxane (10 mL) was added HCl/dioxane (10 mL, 4M in dioxane, 1.72 mmol) and the mixture was stirred at 25° C. for 0.5 h. The mixture was concentrated to give 1-((4-fluoropiperidin-4-yl)methyl)-1H-pyrazole-4-carbonitrile hydrochloride (660 mg).

¹H NMR (400 MHz, CDCl₃) δ_{H} 7.92 (s, 1H), 7.82 (s, 1H), 4.50-4.31 (m, 2H), 3.48-3.45 (d, J=12.0 Hz, 2H), 3.27-3.04 (m, 2H), 2.44-2.13 (m, 2H), 1.85 (m, 2H). ¹⁹F NMR (376.5 MHz, CDCl₃) δ_{F}−163.44

Step 3

To a solution of [2-(pyrimidin-4-yl) pyridine-3-carboxylic acid (317 mg, 1.58 mmol) and HATU (901 mg, 2.37 mmol) in DMF (20 mL) was added DIPEA (1.37 mL, 7.90 mmol) at 20° C. 1-((4-fluoropiperidin-4-yl)methyl)-1H-pyrazole-4-carbonitrile hydrochloride (330 mg, 1.58 mmol) in DMF (20 mL) was added slowly. The mixture was stirred at 20° C. for 1-2 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by flash Prep-HPLC (Column: Phenomenex C18 80*40 mm*3 um; Condition: water (NH₃·H₂O)-ACN; Begin B: 15; End B: 45; Gradient Time (min): 8; 100% B Hold Time (min): 3; FlowRate (ml/min): 30; Injections: 7) provided 1-((4-fluoro-1-(2-(pyrimidin-4-yl)nicotinoyl)piperidin-4-yl)methyl)-1H-pyrazole-4-carbonitrile (134 mg, 21%).

¹H NMR (400 MHz, DMSO-d6) δ_{H} 9.16-9.12 (m, 1H), 8.95-8.84 (m, 1H), 8.76 (m, 1H), 8.32-8.22 (m, 1H), 7.92 (s, 1H), 7.79 (s, 1H), 7.72-7.63 (m, 1H), 7.46 (m, 1H), 4.80-4.51 (m, 1H), 4.46-4.30 (m, 2H), 3.60-3.11 (m, 3H), 2.09-1.69 (m, 3H), 1.49-1.29 (m, 1H). ¹⁹F NMR (376.5 MHz, CDCl3) δ_{F} −163.92 (s, 1F). LCMS purity 99.8%, MS ESI calcd. For C₂₀H₁₈FN₇O [M+H]⁺ 392.1, found 392.1.

### Example 69 and Example 70. Synthesis of (R)-[2,4′-bipyridin]-3-yl(4-fluoro-4-(1-(6-(trifluoromethyl)pyridin-3-yl)ethyl)piperidin-1-yl)methanone (Cmpd 67) and Synthesis of (S)-[2,4′-bipyridin]-3-yl(4-fluoro-4-(1-(6-(trifluoromethyl)pyridin-3-yl)ethyl)piperidin-1-yl)methanone (Cmpd 70)

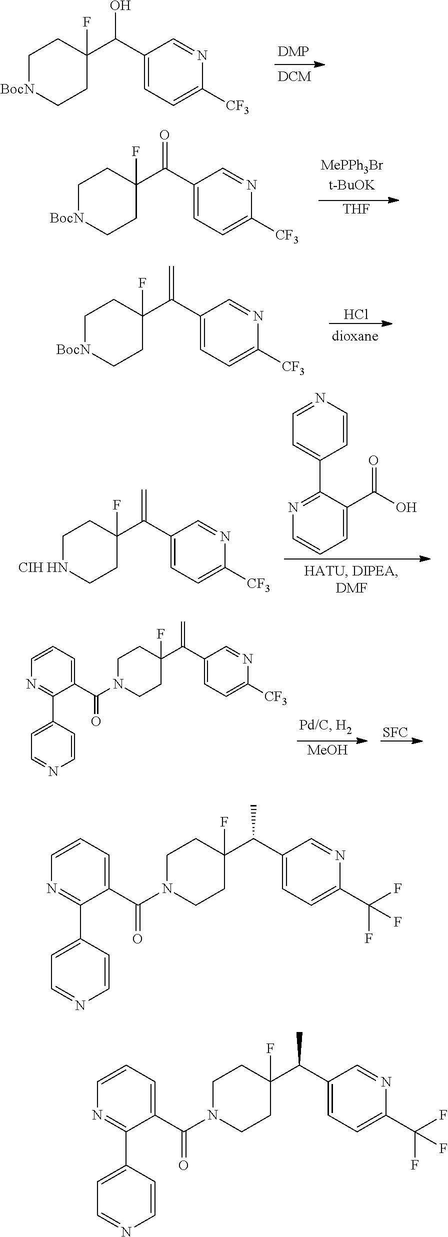

Step 1

To a solution of tert-butyl 4-fluoro-4-(hydroxy(6-(trifluoromethyl)pyridin-3-yl)methyl)piperidine-1-carboxylate (4 g, 10.5 mmol) in DCM (40 mL) was added DMP (8.90 g, 21.0 mmol) at 0° C. under N₂. The mixture was stirred at 25° C. for 30 min. The mixture was poured into NaHCO₃ and Na₂S₂O₃ (200 mL) and stirred for 20 min. The aqueous phase was extracted with DCM (3×50 mL). The combined organic phase was washed with saturated brine (2×50 mL), dried over anhydrous Na₂SO₄, filtered, and concentrated to give tert-butyl 4-fluoro-4-(6-(trifluoromethyl)nicotinoyl)piperidine-1-carboxylate (4 g).

¹H NMR (400 MHz, CDCl₃) δ_{H} 9.32 (s, 1H), 8.49-8.45 (m, 1H), 7.80-7.78 (d, J=8.0 Hz, 1H), 4.10 (s, 2H), 3.32-3.08 (m, 2H), 2.24-1.99 (m, 4H), 1.48 (s, 9H).

Step 2

To a mixture of MePPh₃Br (5.67 g, 15.9 mmol) in THE (100 mL) was added t-BuOK (2.37 g, 1.2 mmol) at 20° C. under N₂. The resulting mixture was stirred at 50° C. for 30 min. tert-butyl 4-fluoro-4-(6-(trifluoromethyl)nicotinoyl)piperidine-1-carboxylate (4 g, 10.6 mmol) was added in portions at <50° C. The reaction mixture was stirred at 50° C. for 16 hours. The reaction mixture was quenched with saturated NH₄Cl (100 mL) at 15° C. The THF layer was separated. The aqueous layer was extracted with EtOAc (50 mL×2). The combined organic phase was concentrated and the residue was purified by flash silica gel chromatography (0-10% EtOAc in PE) to give tert-butyl 4-fluoro-4-(1-(6-(trifluoromethyl)pyridin-3-yl)vinyl)piperidine-1-carboxylate (1.6 g).

¹H NMR (400 MHz, CDCl₃) δ_{H} 8.64 (s, 1H), 7.85-7.83 (d, J=8.0 Hz, 1H), 7.66-7.64 (d, J=8.0 Hz, 1H), 5.56 (d, J=4.0 Hz, 1H), 5.32 (s, 1H), 4.03 (d, J=12.0 Hz, 2H), 3.15-3.01 (m, 2H), 1.93-1.72 (m, 4H), 1.45 (s, 9H).

Step 3

To a solution of tert-butyl 4-fluoro-4-(1-(6-(trifluoromethyl)pyridin-3-yl)vinyl)piperidine-1-carboxylate (600 mg, 1.60 mmol) in 1,4-dioxane (3 mL, 1.60 mmol) was added hydrogen chloride (3 mL, 12.0 mmol, 4 M in 1,4-dioxane) at 25° C. under N₂ and the reaction mixture was stirred at 25° C. for 2 h. The reaction mixture was concentrated under reduced pressure to give 5-(1-(4-fluoropiperidin-4-yl)vinyl)-2-(trifluoromethyl)pyridine hydrochloride (600 mg).

¹H NMR (400 MHz, CDCl₃) δ_{H} 8.71-8.70 (d, J=4.0 Hz, 1H), 8.05-8.03 (m, 1H), 7.94 (d, J=8.4 Hz, 1H), 5.72-5.71 (d, J=4.0 Hz, 1H), 5.53 (s, 1H), 3.27-3.24 (d, J=12.0 Hz, 2H), 3.12-2.96 (m, 2H), 2.31-2.03 (m, 4H).

Step 4

To a solution of [2, 4′-bipyridine]-3-carboxylic acid (193 mg, 0.965 mmol) and HATU (547 mg, 1.44 mmol) in DMF (2 mL) was added DIPEA (0.84 mL, 4.82 mmol). 5-(1-(4-fluoropiperidin-4-yl)vinyl)-2-(trifluoromethyl)pyridine hydrochloride (300 mg, 0.96 mmol) in DMF (2 mL) was added slowly. The mixture was stirred at 20° C. for 2 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated to give [2,4′-bipyridin]-3-yl(4-fluoro-4-(1-(6-(trifluoromethyl)pyridin-3-yl)vinyl)piperidin-1-yl)methanone (200 mg).

Step 5

To a solution of [2,4′-bipyridin]-3-yl(4-fluoro-4-(1-(6-(trifluoromethyl)pyridin-3-yl)vinyl)piperidin-1-yl)methanone (300 mg, 0.6572 mmol) in THE (5 mL) was added Pd/C (wet, 50 mg) at 25° C. Then the solution was hydrogenated under 15 psi of hydrogen at 25° C. for 16 h. The mixture was filtered through a pad of celite and washed with THE (3×5 mL) and the filtrate was concentrated. Purification by flash column (0-30% of EtOAc in PE) provided [2,4′-bipyridin]-3-yl(4-fluoro-4-(1-(6-(trifluoromethyl)pyridin-3-yl)ethyl)piperidin-1-yl)methanone (60 mg). [2,4′-bipyridin]-3-yl(4-fluoro-4-(1-(6-(trifluoromethyl)pyridin-3-yl)ethyl)piperidin-1-yl)methanone (60 mg, 0.1308 mmol) enantiomers were separated by SFC (Column: DAICEL CHIRALPAK AD (250 mm*30 mm, 10 um); Condition: 0.1% NH₃H₂O ETOH; Begin B: 25; End B: 25; FlowRate(ml/min): 60; Injections: 60) to give (R)-[2,4′-bipyridin]-3-yl(4-fluoro-4-(1-(6-(trifluoromethyl)pyridin-3-yl)ethyl)piperidin-1-yl)methanone (11.9 mg, 20%) and (S)-[2,4′-bipyridin]-3-yl(4-fluoro-4-(1-(6-(trifluoromethyl)pyridin-3-yl)ethyl)piperidin-1-yl)methanone (6.8 mg, 11%). Cmpd 67. (R)-[2,4′-bipyridin]-3-yl(4-fluoro-4-(1-(6-(trifluoromethyl)pyridin-3-yl)ethyl) piperidin-1-yl)methanone: ¹H NMR (400 MHz, CDCl₃) δ_{H} 8.92-8.66 (m, 3H), 8.54-8.35 (m, 1H), 7.77-7.61 (m, 5H), 7.44 (m, 1H), 4.81-4.53 (m, 1H), 3.17-2.53 (m, 3H), 2.02 (s, 1H), 1.69-1.54 (m, 2H), 1.43-1.02 (m, 4H), 0.87 (s, 1H). ¹⁹F NMR (376.5 MHz, CDCl₃) δ_{F} −67.869. LCMS purity 99%, MS ESI calcd. for C₂₄H₂₂F₄N₄O [M+H]⁺ 459.2, found 459.2. Cmpd 70. (S)-[2,4′-bipyridin]-3-yl(4-fluoro-4-(1-(6-(trifluoromethyl)pyridin-3-yl)ethyl) piperidin-1-yl)methanone: ¹H NMR (400 MHz, CDCl₃) δ_{H} 8.88-8.65 (m, 3H), 8.53-8.41 (m, 1H), 7.86 (s, 2H), 7.77 (m, 1H), 7.70-7.56 (m, 2H), 7.46 (m, 1H), 4.77-4.55 (m, 1H), 3.13-2.55 (m, 4H), 2.06 (s, 1H), 1.70-1.40 (m, 1H), 1.37-1.17 (m, 4H), 1.06-0.60 (m, 1H) ¹⁹F NMR (376.5 MHz, CDCl₃) δ_{F} −67.871. LCMS purity 99%, MS ESI calcd. for C₂₄H₂₂F₄N₄O [M+H]⁺ 459.2, found 459.2.

### Example 71. Synthesis of (4-((6, 7-dihydro-5H-cyclopenta[b]pyridin-3-yl)methyl)-4-fluoropiperidin-1-yl)(2-(pyrimidin-4-yl)pyridin-3-yl)methanone (Cmpd 68)

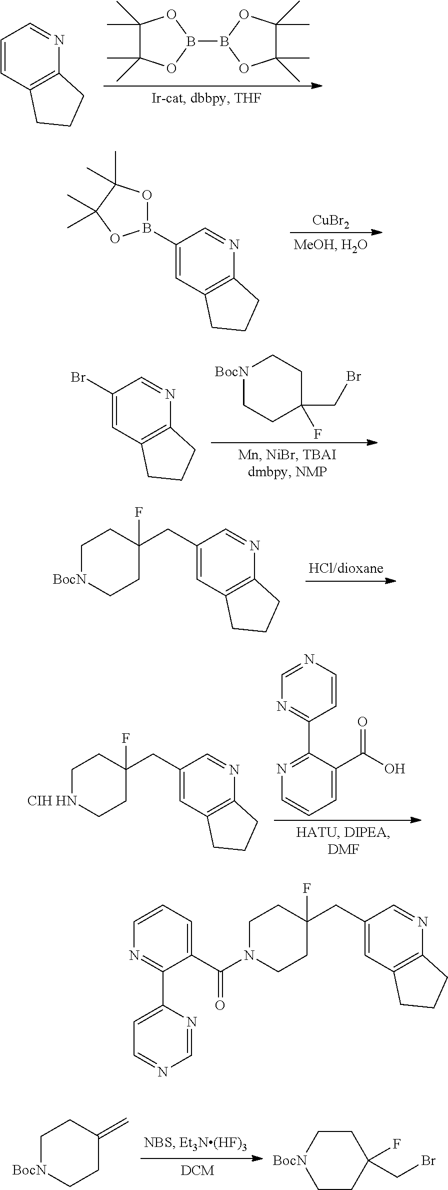

Step 1

To a solution of 6,7-dihydro-5H-cyclopenta[b]pyridine (1 g, 8.39 mmol) in THE (20 mL) was added Ir-cat (166 mg, 0.251 mmol), dbbpy (135 mg, 0.203 mmol) and 4,4,5,5-tetramethyl-2-(4,4,5,5-tetramethyl-1,3,2-dioxaborolan-2-yl)-1,3,2-dioxaborolane (2.34 g, 9.22 mmol) at 20° C. under N₂. The mixture was stirred at 75° C. for 16 h. The mixture was concentrated to give 3-(4,4,5,5-tetramethyl-1,3,2-dioxaborolan-2-yl)-6,7-dihydro-5H-cyclopenta[b]pyridine (2 g), which was used directly in the next step.

Step 2

The 3-(4,4,5,5-tetramethyl-1,3,2-dioxaborolan-2-yl)-6,7-dihydro-5H-cyclopenta[b]pyridine (2 g, 8.15 mmol) was dissolved in methanol (40 mL) and CuBr₂ (6.36 g, 28.5 mmol) in water (40 mL) was added at 25° C. The mixture was heated to 75° C. for 4 hours. The mixture was cooled to room temperature and 10% aqueous ammonia (100 mL) was added. The mixture was extracted with EtOAc (100 mL×3), dried over anhydrous sodium sulfate, filtered and concentrated. Purification by flash column (0-40% of EtOAc in PE) afforded 3-bromo-6,7-dihydro-5H-cyclopenta[b]pyridine (500 mg).

¹H NMR (400 MHz, CDCl₃) δ_{H} 8.12 (d, J=6.0 Hz, 1H), 7.22 (d, J=6.0 Hz, 1H), 3.12 (m, 2H), 2.98 (m, 2H), 2.15 (m, 2H).

Step 3

To a mixture of tert-butyl 4-methylenepiperidine-1-carboxylate (12.5 g, 63.3 mmol) and triethylamine trihydrofluoride (25.4 g, 158 mmol) in DCM (200 mL) was added NBS (16.8 g, 94.9 mmol) at 0° C. After stirring at 20° C. for 3 h, the mixture was poured into ice-water (200 mL), neutralized with aqueous 28% ammonia and extracted with DCM (2×200). The combined extracts were washed with 0.1 N HCl and with 5% aqueous sodium hydrogencarbonate solution, dried with sodium sulfate, filtered, and concentrated. Purification by flash column (0-10% of EtOAc in PE) provided tert-butyl 4-(bromomethyl)-4-fluoropiperidine-1-carboxylate (17.5 g).

¹H NMR (400 MHz, CDCl₃) δ_{H} 3.99 (d, J=5.6 Hz, 2H), 3.45 (d, J=18.0 Hz, 2H), 3.06 (t, J=12.4 Hz, 2H), 2.00-1.90 (m, 2H), 1.79-1.60 (m, 2H), 1.46 (s, 9H). ¹⁹F NMR (376.5 MHz, CDCl₃) δ_{F} −162.37.

Step 4

To a solution 3-bromo-6,7-dihydro-5H-cyclopenta[b]pyridine (500 mg, 2.52 mmol) and tert-butyl 4-(bromomethyl)-4-fluoropiperidine-1-carboxylate (894 mg, 3.02 mmol) in NMP (8 mL) was added NiBr₂ (218 mg, 1.00 mmol) and Mn (1.10 g, 20.1 mmol) and TBAI (742 mg, 2.01 mmol) in one portion at 20° C. Then the mixture was stirred at 80° C. for 24 hours. The reaction mixture was filtered and poured into water (20 mL). The aqueous phase was extracted with EtOAc (3×20 mL). The combined organic phase was washed with brine (2×20 mL), dried over anhydrous Na₂SO₄, filtered, and concentrated. Purification by flash column (0-50% of EtOAc in PE) provided tert-butyl 4-((6,7-dihydro-5H-cyclopenta[b]pyridin-3-yl)methyl)-4-fluoropiperidine-1-carboxylate (300 mg).

¹H NMR (400 MHz, CDCl₃) δ_{H} 8.27 (d, J=6.0 Hz, 1H), 6.96 (d, J=6.0 Hz, 1H), 4.03-3.87 (m, 2H), 3.11 (m, 2H), 3.05-2.98 (m, 2H), 2.97-2.89 (m, 3H), 2.87-2.82 (m, 1H), 2.13 (m, 2H), 1.81-1.71 (m, 4H), 1.45 (s, 9H). ¹⁹F NMR (376.5 MHz, CDCl₃) δ_{F} −161.12.

Step 5

To a solution of tert-butyl 4-((6,7-dihydro-5H-cyclopenta[b]pyridin-3-yl)methyl)-4-fluoropiperidine-1-carboxylate (300 mg, 0.897 mmol) in 1,4-dioxane (5 mL) was added hydrogen chloride (10 mL, 40.0 mmol, 4 M in 1,4-dioxane) at 20° C. under N₂ and the reaction mixture was stirred at 20° C. for 1 h. The reaction mixture was concentrated under reduced pressure to give 3-((4-fluoropiperidin-4-yl)methyl)-6,7-dihydro-5H-cyclopenta[b]pyridine hydrochloride (300 mg).

Step 6

To a solution of 2-(pyrimidin-4-yl)pyridine-3-carboxylic acid (89.9 mg, 0.442 mmol) and HATU (210 mg, 0.553 mmol) in DMF (3 mL) was added DIPEA (0.32 mL, 1.87 mmol, 0.74 g/mL) at 20° C. 3-((4-fluoropiperidin-4-yl)methyl)-6,7-dihydro-5H-cyclopenta[b]pyridine hydrochloride (100 mg, 0.369 mmol) in DMF (2 mL) was added slowly at 20° C. The mixture was stirred at 20° C. for 1 h. The reaction mixture was poured into H₂O (10 mL) and stirred for 20 min. The aqueous phase was extracted with EtOAc (3×10 mL). The combined organic phase was washed with brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 25; End B: 55; Gradient Time(min): 8; 100% B Hold Time(min): 2; FlowRate(ml/min): 30; Injections: 6) provided (4-((6,7-dihydro-5H-cyclopenta[b]pyridin-3-yl)methyl)-4-fluoropiperidin-1-yl)(2-(pyrimidin-4-yl)pyridin-3-yl)methanone (31 mg, 20%).

¹H NMR (400 MHz, CDCl₃) δ_{H} 8.93-8.83 (m, 2H), 8.75 (dd, J=1.6, 4.8 Hz, 1H), 8.30-8.22 (m, 2H), 7.73-7.64 (m, 1H), 7.45 (m, 1H), 6.89 (d, J=5.2 Hz, 1H), 4.78-4.54 (m, 1H), 3.49-3.33 (m, 1H), 3.27-3.08 (m, 2H), 3.05 (t, J=7.6 Hz, 2H), 2.99-2.82 (m, 4H), 2.10 (t, J=7.2 Hz, 2H), 2.02-1.87 (m, 2H), 1.66-1.51 (m, 2H). ¹⁹F NMR (376.5 MHz, CDCl₃). δ_{F} −160.80. LCMS purity 98%, MS ESI calcd. For C₂₄H₂₄FN₅O [M+H]⁺ 418.2, found 418.2.

### Example 72. Synthesis of [2,4′-bipyridin]-3-yl(4-((6, 7-dihydro-5H-cyclopenta[b]pyridin-3-yl)methyl)-4-fluoropiperidin-1-yl)methanone (Cmpd 69)

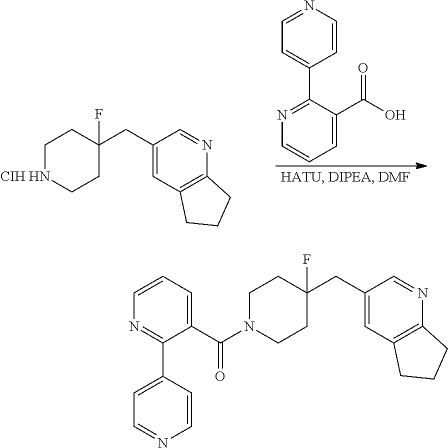

To a solution of [2,4′-bipyridine]-3-carboxylic acid (88.4 mg, 0.442 mmol) and HATU (210 mg, 0.553 mmol) in DMF (3 mL) was added DIPEA (0.32 mL, 1.84 mmol) at 20° C. and stirred for 15 min. 3-((4-fluoropiperidin-4-yl)methyl)-6,7-dihydro-5H-cyclopenta[b]pyridine hydrochloride (100 mg, 0.369 mmol) in DMF (3 mL) was added slowly into the mixture. The mixture was stirred at 20° C. for 1 h. The reaction mixture was poured into H₂O (10 mL) and stirred for 20 min. The aqueous phase was extracted with EtOAc (3×10 mL). The combined organic phase was washed with brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 23; End B: 53; Gradient Time(min): 8; 100% B Hold Time(min): 2; FlowRate(ml/min): 30; Injections: 6) provided [2,4′-bipyridin]-3-yl(4-((6,7-dihydro-5H-cyclopenta[b]pyridin-3-yl)methyl)-4-fluoropiperidin-1-yl)methanone (25 mg, 16%).

¹H NMR (400 MHz, CDCl₃) δ_{H} 8.79 (dd, J=1.6, 4.8 Hz, 1H), 8.76 (d, J=5.2 Hz, 1H), 8.69 (d, J=5.2 Hz, 1H), 8.23 (d, J=5.2 Hz, 1H), 7.88-7.71 (m, 2H), 7.60 (d, J=4.4 Hz, 1H), 7.43 (dd, J=4.8, 7.6 Hz, 1H), 6.86-6.73 (m, 1H), 4.74-4.53 (m, 1H), 3.09-2.98 (m, 3H), 2.97-2.76 (m, 4H), 2.75-2.43 (m, 2H), 2.18-2.01 (m, 2H), 1.89 (s, 1H), 1.72-1.62 (m, 1H), 1.48-1.16 (m, 2H). LCMS purity 99%, MS ESI calcd. For C₂₅H₂₅FN₄O [M+H]⁺ 417.2, found 417.2.

### Example 73. Synthesis of (4-((5-chloropyridin-2-yl)methyl)-4-fluoropiperidin-1-yl)(3-(pyridin-4-yl)pyrazin-2-yl)methanone (Cmpd 70)

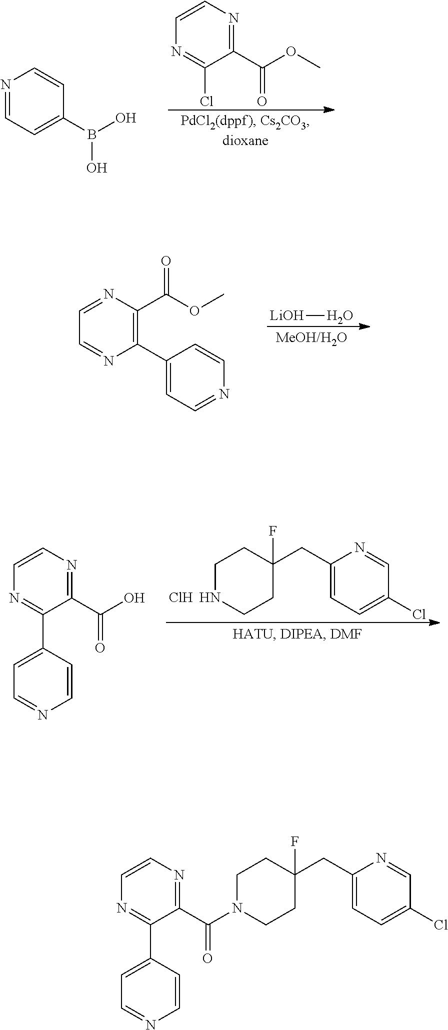

Step 1

To a solution of (pyridin-4-yl)boronic acid (1.0 g, 8.13 mmol) in dioxane (20 mL) under N₂ at 20° C. was added PdCl₂(dppf) (59.6 mg, 0.0813 mmol), methyl 3-chloropyrazine-2-carboxylate (1.26 g, 7.31 mmol) and Cs₂CO₃ (5.27 g, 16.2 mmol) was added dropwise at 20° C. under N₂. The mixture was stirred at 20° C. for 0.5 h and at 75° C. for 12 h. The mixture was poured into 10% NH₄Cl (100 mL) and extracted with EtOAc (2×50 mL). The combined organic layer was washed with brine (100 mL), dried over Na₂SO₄, filtered, and concentrated. Purification by combi-flash (10-50% of EtOAc in PE) provided methyl 3-(pyridin-4-yl)pyrazine-2-carboxylate (300 mg, 17%).

¹H NMR (400 MHz, CDCl₃) δ_{H} 8.83-8.82 (m, 1H), 8.77-8.75 (m, 2H), 8.72-8.71 (m, 1H), 7.64-7.45 (m, 2H), 3.89 (s, 3H).

Step 2

To a mixture of methyl 3-(pyridin-4-yl)pyrazine-2-carboxylate (230 mg, 1.06 mmol) in MeOH (5 mL)/H₂O (1 mL) was added LiOH H₂O (48.7 mg, 1.16 mmol) and the mixture was stirred at 60° C. for 16 h. The reaction mixture was concentrated to dryness and then triturated with DCM (20 mL) at 20° C. for 1 h and then filtered. The filter cake was washed with DCM (20 mL) and dried in vacuum to give 3-(pyridin-4-yl)pyrazine-2-carboxylic acid (200 mg).

¹H NMR (400 MHz, CD₃OD) δ_{H} 8.68-8.67 (m, 1H), 8.64-8.62 (m, 2H), 8.55-8.54 (m, 1H), 7.94-7.90 (m, 2H).

Step 3

To a solution of 3-(pyridin-4-yl)pyrazine-2-carboxylic acid (100 mg, 0.497 mmol) and HATU (226 mg, 0.596 mmol) in DMF (5 mL) was added DIPEA (0.259 mL, 1.49 mmol). 5-chloro-2-[(4-fluoropiperidin-4-yl)methyl]pyridine hydrochloride (131 mg, 0.497 mmol) in DMF (5 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by Prep-HPLC (Column: Phenomenex C18 80*40 mm*3 um; Condition: water (NH₃H₂O)-ACN; Begin B: 24; End B: 54; Gradient Time (min): 8; 100% B Hold Time (min): 2; FlowRate (ml/min): 30; Injections: 4) provided (4-((5-chloropyridin-2-yl)methyl)-4-fluoropiperidin-1-yl)(3-(pyridin-4-yl)pyrazin-2-yl)methanone (56 mg, 27%).

¹H NMR (400 MHz, CDCl₃) δ_{H} 8.79-8.72 (m, 3H), 8.65-8.64 (m, 1H), 8.51-8.50 (m, 1H), 7.75-7.69 (m, 2H), 7.62-7.59 (m, 1H), 7.17-7.15 (m, 1H), 3.18-2.99 (m, 5H), 1.97-1.80 (m, 2H), 1.73-1.54 (m, 2H), 1.48-1.33 (m, 1H). ¹⁹F NMR (376.5 MHz, CDCl₃) δ_{F} −160.65. LCMS purity 99%; MS ESI calcd. for C₂₁H₁₉ClFN₅O [M+H]⁺ 412.1, found 412.1.

### Example 74. Synthesis of 5-((4-fluoro-1-(2-(pyrimidin-4-yl)nicotinoyl)piperidin-4-yl)methyl)nicotinonitrile (Cmpd 71)

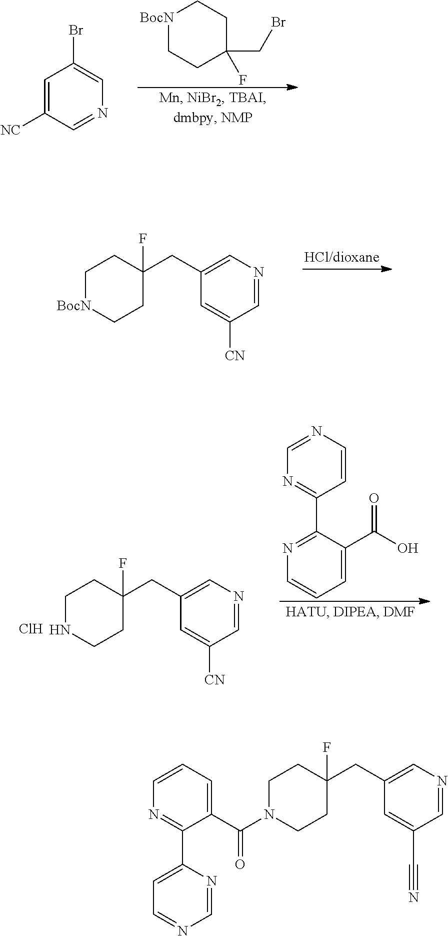

Step 1

To a solution of 5-bromonicotinonitrile (1.0 g, 5.06 mmol) and tert-butyl 4-(bromomethyl)-4-fluoropiperidine-1-carboxylate (1.61 g, 5.46 mmol) in NMP (10 mL) was added nickel dibromide (119 mg, 0.546 mmol), manganese (1.19 g, 21.8 mmol), tetrabutylazanium iodide (201 mg, 0.546 mmol), and dmbpy (200 mg, 1.09 mmol) in one portion at 80° C. under N₂. The mixture was stirred for 12 hours, cooled, poured into NH₄Cl (50 mL), and stirred for 20 min. The aqueous phase was extracted with EtOAc (2×30 mL). The combined organic phase was washed with saturated brine (2×50 mL), dried over anhydrous Na₂SO₄, filtered and concentrated. Purification by flash column (10-50% of EtOAc in PE) provided tert-butyl 4-((5-cyanopyridin-3-yl)methyl)-4-fluoropiperidine-1-carboxylate (0.6 g). The product was combined with another batch of tert-butyl 4-((5-cyanopyridin-3-yl)methyl)-4-fluoropiperidine-1-carboxylate (1.4 g) prepared under the same conditions and purified by SFC (Column: Phenomenex C18 80*40 mm*3 um; Condition: water (NH₃H₂O)-ACN; Begin B: 39; End B: 69; Gradient Time(min): 8; 100% B Hold Time(min): 3; FlowRate(ml/min): 30; Injections: 11) to give tert-butyl 4-((5-cyanopyridin-3-yl)methyl)-4-fluoropiperidine-1-carboxylate (200 mg, 10.0%).

¹H NMR (400 MHz, CDCl₃) δ_{H} 8.80 (s, 1H), 8.64-8.63 (m, 1H), 7.85 (s, 1H), 3.96 (br, 2H), 3.07-2.99 (m, 2H), 2.98-2.90 (m, 2H), 1.68-1.66 (m, 4H), 1.45 (s, 9H).

Step 2

To a mixture of tert-butyl 4-((5-cyanopyridin-3-yl)methyl)-4-fluoropiperidine-1-carboxylate (180 mg, 0.56 mmol) in dioxane (10 mL) was added HCl/dioxane (1.40 mL, 4M in dioxane, 5.63 mmol) and the mixture was stirred at 25° C. for 2 h. The mixture was cooled and concentrated to give 5-((4-fluoropiperidin-4-yl)methyl)nicotinonitrile hydrochloride (200 mg), which was carried directly on to the next step.

Step 3

To a solution of 2-(pyrimidin-4-yl)pyridine-3-carboxylic acid (94.3 mg, 0.46 mmol) and HATU (193 mg, 0.51 mmol) in DMF (5 mL) was added DIPEA (0.20 mL, 1.17 mmol). 5-((4-fluoropiperidin-4-yl)methyl)nicotinonitrile hydrochloride (100 mg, 0.39 mmol) in DMF (5 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by Prep-HPLC (Column: Phenomenex C18 80*40 mm*3 um; Condition: water (NH₃H₂O)-ACN; Begin B: 21; End B: 51; Gradient Time(min): 8; 100% B Hold Time(min): 2.3; FlowRate(ml/min): 30; Injections: 4) provided 5-((4-fluoro-1-(2-(pyrimidin-4-yl)nicotinoyl)piperidin-4-yl)methyl)nicotinonitrile (25 mg, 16%).

¹H NMR (400 MHz, CDCl₃) δ_{H} 9.22-9.04 (m, 1H), 8.90 (m, 1H), 8.82 (s, 1H), 8.77 (m, 1H), 8.65 (br s, 1H), 8.28 (m, 1H), 7.86 (s, 1H), 7.74-7.63 (m, 1H), 7.47 (m, 1H), 4.82-4.57 (m, 1H), 3.51-3.19 (m, 2H), 3.16-2.90 (m, 3H), 2.04-1.82 (m, 2H), 1.74-1.70 (m, 1H), 1.55-1.44 (m, 1H). ¹⁹F NMR (376.5 MHz, CDCl₃) δ_{F} −166.63. LCMS purity 95%; MS ESI calcd. for C₂₂H₁₉FN₆O [M+H]⁺ 403.2, found 403.2.

### Example 75. Synthesis of 5-((1-([2,4′-bipyridine]-3-carbonyl)-4-fluoropiperidin-4-yl)methyl)nicotinonitrile (Cmpd 72)

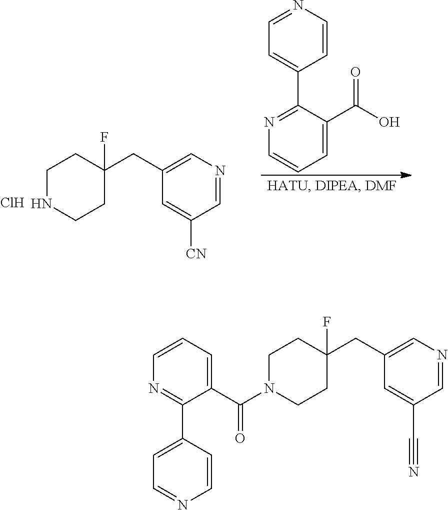 [SMILES: Cl ; N#Cc1cncc(CC2(F)CCNCC2)c1 ; O=C(O)c1cccnc1-c1ccncc1 ; N#Cc1cncc(CC2(F)CCN(C(=O)c3cccnc3-c3ccncc3)CC2)c1]

To a solution of [2,4′-bipyridine]-3-carboxylic acid (93.8 mg, 0.469 mmol) and HATU (193 mg, 0.508 mmol) in DMF (5 mL) was added DIPEA (0.204 mL, 1.17 mmol). 5-((4-fluoropiperidin-4-yl)methyl)nicotinonitrile hydrochloride (100 mg, 0.391 mmol) in DMF (5 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by Prep-HPLC (Column: Phenomenex C18 80*40 mm*3 um; Condition: water (NH₃H₂O)-ACN; Begin B: 20; End B: 50; Gradient Time(min): 8; 100% B Hold Time(min): 2; FlowRate(ml/min): 30; Injections: 4) provided 5-((1-([2,4′-bipyridine]-3-carbonyl)-4-fluoropiperidin-4-yl)methyl)nicotinonitrile (10 mg, 7%).

¹H NMR (400 MHz, CDCl₃) δ_{H} 8.88-8.56 (m, 5H), 7.90-7.61 (m, 4H), 7.46-7.43 (m, 1H), 4.97-4.73 (m, 1H), 3.28-3.02 (m, 3H), 2.95-2.66 (m, 2H), 2.04-1.88 (m, 1H), 1.67-1.41 (m, 1H), 1.34-1.23 (m, 1H), 0.15-0.06 (m, 1H). ¹⁹F NMR (376.5 MHz, CDCl₃) δ_{F} −164.63. LCMS purity 99%; MS ESI calcd. for C₂₃H₂₀FN₅O [M+H]⁺ 402.3, found 402.3.

### Example 76. 1-((1-([2,4′-bipyridine]-3-carbonyl)-4-fluoropiperidin-4-yl)methyl)-1H-pyrazole-3-carbonitrile (Cmpd 74)

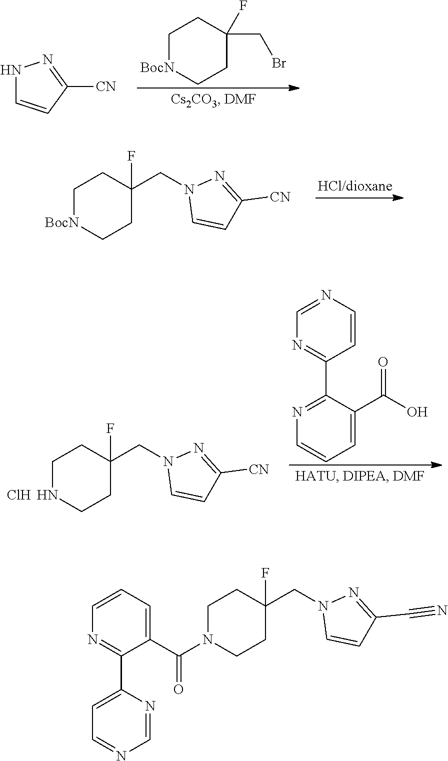

Step 1

To a solution 1H-pyrazole-3-carbonitrile (500 mg, 5.37 mmol) and tert-butyl 4-(bromomethyl)-4-fluoropiperidine-1-carboxylate (1.59 g, 5.37 mmol) in DMF (20 mL) was added Cs₂CO₃ in one portion at 80° C. under N₂ and then stirred for 48 h. The mixture was poured into water and aqueous LiCi (20 mL) and stirred for 20 mins. The aqueous phase was extracted with EtOAc (2×20 mL). The combined organic phase was washed with brine (2×20 mL), dried over Na₂SO₄, filtered, and concentrated. The residue was purified by flash column to give tert-butyl 4-((3-cyano-1H-pyrazol-1-yl)methyl)-4-fluoropiperidine-1-carboxylate (600 mg).

¹H NMR (400 MHz, CDCl₃) δ_{H} 7.68 (d, 1H), 6.77 (d, 1H), 4.50-4.28 (m, 2H), 3.97 (s, 2H), 3.12-2.98 (m, 2H), 1.72-1.61 (m, 4H), 1.46 (s, 9H). ¹⁹F NMR (376.5 MHz, CDCl₃) δ_{F} −163.167.

Step 2

To a mixture of tert-butyl 4-((3-cyano-1H-pyrazol-1-yl)methyl)-4-fluoropiperidine-1-carboxylate (400 mg, 1.29 mmol) in dioxane (6 mL) was added HCl/dioxane (8 mL, 4M in dioxane, 32.0 mmol) and the mixture was stirred at 25 C for 0.5 h. The mixture was concentrated to give 1-((4-fluoropiperidin-4-yl)methyl)-1H-pyrazole-3-carbonitrile hydrochloride which was carried directly to the next step.

Step 3

To a solution of [2,4′-bipyridine]-3-carboxylic acid (163 mg, 0.817 mmol) and HATU (463 mg, 1.22 mmol) in DMF (8 mL) was added DIPEA (0.712 mL, 4.08 mmol). 1-((4-fluoropiperidin-4-yl)methyl)-1H-pyrazole-3-carbonitrile hydrochloride (200 mg, 0.817 mmol) in DMF (2 mL) was added slowly. The mixture was stirred at 20 C for 1 h. The reaction mixture was poured into H₂O (10 mL) and stirred for 20 min. The aqueous phase was extracted with EtOAc (3×10 mL). The combined organic phase was washed with brine (2×10 mL), dried over Na₂SO₄, filtered, and concentrated. Purification by HPLC (Column: Phenomenex C18 80*40 mm*3 um; Condition: water (NH₃H₂O)-ACN; Begin B: 19; End B: 49; Gradient Time(min): 8; 100% B Hold Time(min): 2; FlowRate(ml/min): 30; Injections: 3) provided 1-((1-([2,4′-bipyridine]-3-carbonyl)-4-fluoropiperidin-4-yl)methyl)-1H-pyrazole-3-carbonitrile (68 mg, 21%).

¹H NMR (400 MHz, CDCl₃) S_{H} 8.83-8.67 (m, 1H), 8.72 (d, 1H), 7.75 (s, 2H), 7.60 (d, 1H), 7.53-7.39 (m, 2H), 6.85-6.63 (m, 1H), 4.63 (d, 1H), 4.50-3.91 (m, 2H), 3.02 (d, 2H), 2.90-2.49 (m, 1H), 1.74 (d, 1H), 1.52-1.06 (m, 3H), 0.32-0.00 (m, 1H). LCMS purity 99%; MS ESI calcd. for C₂₁H₁₉FN₆O [M+H]⁺ 391.1, found 391.1.

### Example 77. Synthesis of 1-((4-fluoro-1-(2-(pyrimidin-4-yl)nicotinoyl)piperidin-4-yl)methyl)-1H-pyrazole-3-carbonitrile (Cmpd 73)

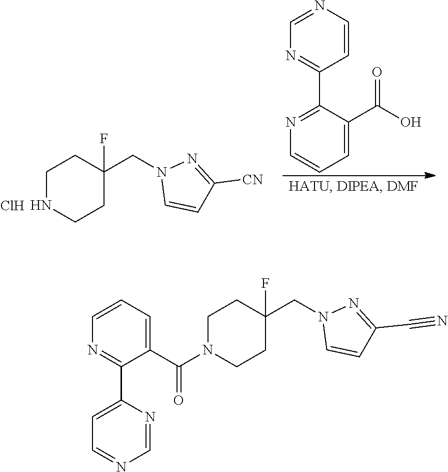 [SMILES: Cl ; N#Cc1ccn(CC2(F)CCNCC2)n1 ; O=C(O)c1cccnc1-c1ccncn1 ; N#Cc1ccn(CC2(F)CCN(C(=O)c3cccnc3-c3ccncn3)CC2)n1]

To a solution of 2-(pyrimidin-4-yl)pyridine-3-carboxylic acid (147 mg, 0.734 mmol) and HATU (349 mg, 0.918 mmol) in DMF (4 mL) was added DIPEA (0.533 mL, 3.06 mmol, 0.74 g/mL) at 20° C. and stirred for 15 min. 1-((4-fluoropiperidin-4-yl)methyl)-1H-pyrazole-3-carbonitrile hydrochloride (150 mg, 0.612 mmol) in DMF (4 mL) was added slowly at 20° C. The mixture was stirred at 20° C. for 1 h. The reaction mixture was poured into H₂O (10 mL) and stirred for 20 mins. The aqueous phase was extracted with EtOAc (3×10 mL). The combined organic phase was washed with brine (2×10 mL), dried over Na₂SO₄, filtered, and concentrated. Purification by HPLC (Column: Phenomenex C18 80*40 mm*3 um; Condition: water (NH₃H₂O)-ACN; Begin B: 22; End B: 52; Gradient Time(min): 8; 100% B Hold Time(min): 3; FlowRate(ml/min): 30; Injections 4) provided 1-((4-fluoro-1-(2-(pyrimidin-4-yl)nicotinoyl)piperidin-4-yl)methyl)-1H-pyrazole-3-carbonitrile (59 mg, 25%).

¹H NMR (400 MHz, CDCl₃) S_{H} 9.21-9.10 (m, 1H), 8.96-8.84 (m, 1H), 8.76 (m, 1H), 8.27 (s, 1H), 7.68 (m, 1H), 7.56 (s, 1H), 7.51-7.43 (m, 1H), 6.72 (d, 1H), 4.75-4.50 (m, 2H), 4.44-4.33 (m, 2H), 3.54-3.11 (m, 4H), 1.92 (d, 1H), 1.56-1.35 (m, 2H). ¹⁹F NMR (376.5 MHz, CDCl₃) S_{F}-163.803. LCMS: purity 99%; MS ESI calcd. for C₂₀H₁₈FN₇O [M+H]⁺ 392.2, found 392.2.

### Example 78. Synthesis of [2,4′-bipyridin]-3-yl(4-fluoro-4-((2-methylthiazol-5-yl)methyl)piperidin-1-yl)methanone (Cmpd 75)

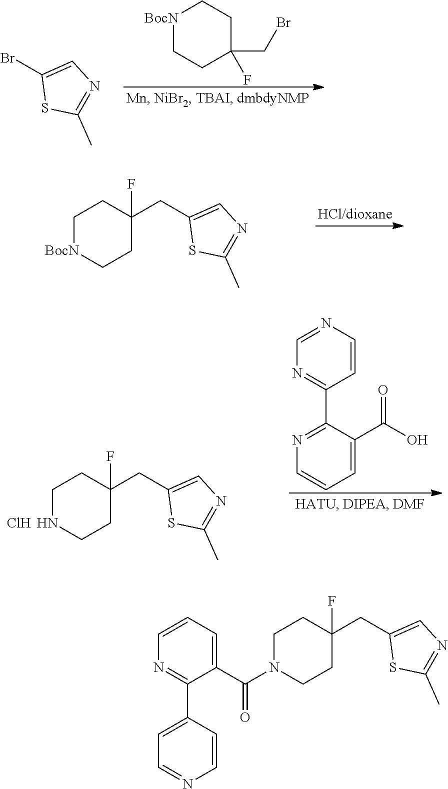

Step 1

To a solution 5-bromo-2-methylthiazole (2 g, 11.2 mmol) and tert-butyl 4-(bromomethyl)-4-fluoropiperidine-1-carboxylate (3.31 g, 11.2 mmol) in NMP (20 mL) was added nickel dibromide (244 mg, 1.12 mmol), manganese (2.46 g, 44.8 mmol), tetrabutylazanium iodide (413 mg, 1.12 mmol), and dmbpy (412 mg, 2.24 mmol) in one portion at 80° C. The mixture was stirred under N₂ for 12 h and then concentrated. The residue was purified by flash column (0˜50% of EtOAc in PE) to give tert-butyl 4-fluoro-4-((2-methylthiazol-5-yl)methyl)piperidine-1-carboxylate (600 mg).

¹H NMR (400 MHz, CDCl₃) δ_{H} 7.35 (s, 1H), 3.93 (s, 2H), 3.14-2.97 (m, 4H), 2.69-2.65 (m, 3H), 1.94 (s, 2H), 1.79-1.74 (m, 2H), 1.44 (s, 9H)

Step 2

To a solution of tert-butyl 4-fluoro-4-((2-methylthiazol-5-yl)methyl)piperidine-1-carboxylate (600 mg, impure) in 1,4-dioxane (3 mL) was added hydrogen chloride (3 mL, 12.0 mmol, 4 M in 1,4-dioxane) at 25° C. under N₂ atmosphere and the reaction mixture was stirred at 25° C. for 2 h. The reaction mixture was concentrated under reduced pressure to give 5-((4-fluoropiperidin-4-yl)methyl)-2-methylthiazole hydrochloride (200 mg), which was carried directly to the next step.

Step 3

To a solution of [2, 4′-bipyridine]-3-carboxylic acid (28.6 mg, 143 μmol) and HATU (68 mg, 179 μmol) in DMF (2 mL) was added DIPEA (0.104 mL, 0.598 mmol). 5-((4-fluoropiperidin-4-yl)methyl)-2-methylthiazole hydrochloride (30 mg, 0.1196 mmol) in DMF (2 mL) was added slowly. The mixture was stirred at 20° C. for 2 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 20; End B: 50; Gradient Time(min): 8; 100% BHoldTime(min): 2; Flow Rate (ml/min): 30; Injections: 4) afforded [2,4′-bipyridin]-3-yl(4-fluoro-4-((2-methylthiazol-5-yl)methyl)piperidin-1-yl)methanone (7.9 mg, 16%).

¹H NMR (400 MHz, CDCl₃) δ_{H} 8.84-8.65 (m, 3H), 7.81-7.71 (m, 2H), 7.61 (d, J=4.8 Hz, 1H), 7.45-7.41 (m, 1H), 7.23 (s, 1H), 4.69-4.56 (m, 1H), 3.07-2.73 (m, 4H), 2.67-2.63 (m, 3H), 1.99-1.64 (m, 2H), 1.47-1.12 (m, 2H), 0.21-0.12 (m, 1H). ¹⁹F NMR (376.5 MHz, CDCl₃) δ_{F} −163.605. LCMS purity >98%, MS ESI calcd. for C₂₁H₂₁FN₄OS [M+H]⁺ 397.1, found 397.1.

### Example 79. Synthesis of (4-fluoro-4-((2-methylthiazol-5-yl)methyl)piperidin-1-yl)(2-(pyrimidin-4-yl)pyridin-3-yl)methanone (Cmpd 76)

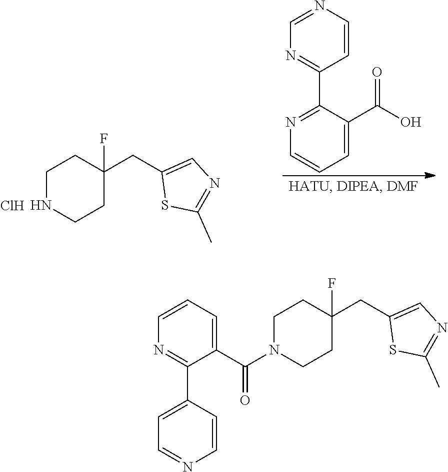 [SMILES: Cl ; Cc1ncc(CC2(F)CCNCC2)s1 ; O=C(O)c1cccnc1-c1ccncn1 ; Cc1ncc(CC2(F)CCN(C(=O)c3cccnc3-c3ccncc3)CC2)s1]

To a solution of 2-(pyrimidin-4-yl)pyridine-3-carboxylic acid (192 mg, 957 μmol) and HATU (452 mg, 1.19 mmol) in DMF (2 mL) was added DIPEA (0.694 mL, 3.98 mmol). 5-((4-fluoropiperidin-4-yl)methyl)-2-methylthiazole hydrochloride (200 mg, 0.7975 mmol) in DMF (2 mL) was added slowly. The mixture was stirred at 20° C. for 2 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by HPLC (Column: Phenomenex C18 80*40 mm*3 um; Condition: water (NH₃H₂O)-ACN; Begin B: 20; End B: 50; Gradient Time(min): 8; 100% B Hold Time(min): 2; Flow Rate (ml/min): 30; Injections: 5) afforded (4-fluoro-4-((2-methylthiazol-5-yl)methyl)piperidin-1-yl)(2-(pyrimidin-4-yl)pyridin-3-yl)methanone (56 mg, 17%).

¹H NMR (400 MHz, CDCl₃) δ_{H} 8.95-8.84 (m, 1H), 9.19 (s, 1H), 8.75-8.73 (m, 1H), 8.29-8.19 (m, 1H), 7.67 (d, J=7.2 Hz, 1H), 7.47-7.43 (m, 1H), 7.35 (s, 1H), 4.75-4.53 (m, 1H), 3.48-3.36 (m, 1H), 3.27-3.02 (m, 4H), 2.71-2.66 (m, 3H), 2.10-1.68 (m, 4H). ¹⁹F NMR (376.5 MHz, CDCl₃) δ_{F} −161.293. LCMS purity >97%, MS ESI calcd. for C₂₀H₂OFN₅OS [M+H]⁺ 398.1, found 398.1.

### Example 80. Synthesis of 5-((1-([2,4′-bipyridine]-3-carbonyl)-4-fluoropiperidin-4-yl)methyl)picolinonitrile (Cmpd 77)

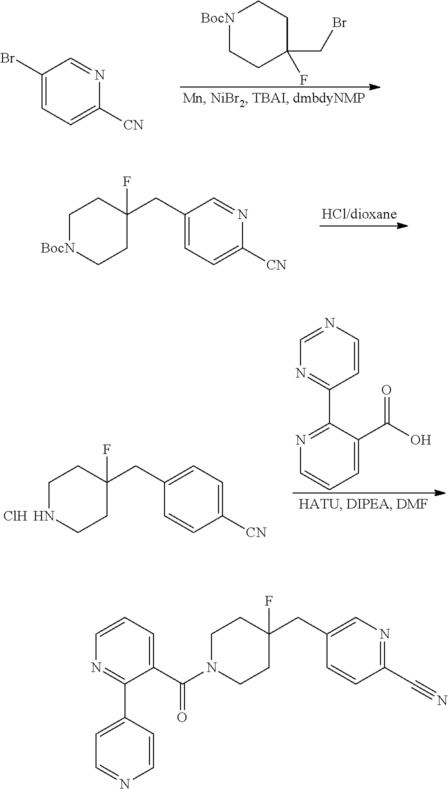

Step 1

To a solution 5-bromopicolinonitrile (2 g, 11.2 mmol) and tert-butyl 4-(bromomethyl)-4-fluoropiperidine-1-carboxylate (3.31 g, 11.2 mmol) in NMP (20 mL) was added nickel dibromide (244 mg, 1.12 mmol), manganese (2.46 g, 44.8 mmol), tetrabutylazanium iodide (413 mg, 1.12 mmol), and dmbpy (412 mg, 2.24 mmol) in one portion at 80° C. under N₂ and then the mixture was stirred for 12 h. The mixture was concentrated and the residue was purified by flash column (0˜50% of EtOAc in PE) to give tert-butyl 4-((6-cyanopyridin-3-yl)methyl)-4-fluoropiperidine-1-carboxylate (600 mg).

Step 2

To a solution of tert-butyl 4-((6-cyanopyridin-3-yl)methyl)-4-fluoropiperidine-1-carboxylate (600 mg, impure) in 1,4-dioxane (3 mL) was added hydrogen chloride (3 mL, 12.0 mmol, 4 M in 1,4-dioxane) at 25° C. under N₂ and the reaction mixture was stirred at 25° C. for 2 h. The reaction mixture was concentrated under reduced pressure to give tert-butyl 4-((6-cyanopyridin-3-yl)methyl)-4-fluoropiperidine-1-carboxylate (200 mg) that was used directly for the next step.

Step 3

To a solution of [2,4′-bipyridine]-3-carboxylic acid (28.6 mg, 0.143 mmol) and HATU (68 mg, 0.179 mmol) in DMF (2 mL) was added DIPEA (0.104 mL, 0.598 mmol). 5-((1-([2,4′-bipyridine]-3-carbonyl)-4-fluoropiperidin-4-yl)methyl)picolinonitrile (30 mg, 0.1196 mmol) in DMF (2 mL) was added slowly. The mixture was stirred at 20° C. for 2 h. The reaction mixture poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 20; End B: 50; Gradient Time(min): 8; 100% BHoldTime(min): 2; Flow Rate (ml/min): 30; Injections: 4) provided 5-((1-([2,4′-bipyridine]-3-carbonyl)-4-fluoropiperidin-4-yl)methyl)picolinonitrile (7.9 mg, 16%) ¹H NMR (400 MHz, CDCl₃) δ_{H} 8.84-8.65 (m, 3H), 8.58-8.40 (m, 1H), 7.78-7.63 (m, 2H), 7.56-7.45 (m, 3H), 7.44-7.40 (m, 1H), 4.69-4.65 (m, 2H), 3.07-2.73 (m, 3H), 2.70-2.63 (m, 2H), 1.60-1.28 (m, 2H), 1.22-1.08 (m, 1H). ¹⁹F NMR (376.5 MHz, CDCl₃) δ_{F} −164.5. LCMS purity >98%, MS ESI calcd. for C₂₁H₂₂FN₄O [M+H]⁺ 402.1, found 402.1.

### Example 81. Synthesis of 5-((4-fluoro-1-(2-(pyrimidin-4-yl)nicotinoyl)piperidin-4-yl)methyl)picolinonitrile (Cmpd 85)

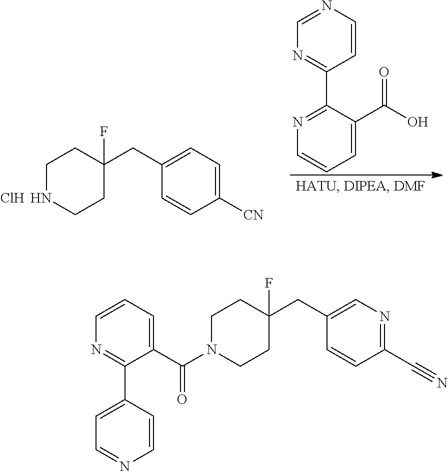 [SMILES: Cl ; N#Cc1ccc(CC2(F)CCNCC2)cc1 ; O=C(O)c1cccnc1-c1ccncn1 ; N#Cc1ccc(CC2(F)CCN(C(=O)c3cccnc3-c3ccncc3)CC2)cn1]

To a solution of 2-(pyrimidin-4-yl)pyridine-3-carboxylic acid (188 mg, 0.938 mmol), HOBT (158 mg, 1.17 mmol) and EDCI (225 mg, 1.17 mmol) in DMF (2 mL) was added DIPEA (0.386 mL, 2.34 mmol) followed by slow addition of 5-((4-fluoropiperidin-4-yl)methyl)picolinonitrile hydrochloride (200 mg, 0.7821 mmol) in DMF (2 mL). The mixture was stirred at 20° C. for 2 h and poured into H₂O (10 mL). The aqueous phase was extracted with EtOAc (3×10 mL). The combined organic phase was washed with brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 20; End B: 50; Gradient Time (min): 8; 100% B HoldTime(min): 3; FlowRate(ml/min): 30; Injections: 5) to give 5-((4-fluoro-1-(2-(pyrimidin-4-yl)nicotinoyl)piperidin-4-yl)methyl)picolinonitrile (26 mg, 6%).

¹H NMR (400 MHz, CDCl₃) S_{H} 9.21-8.96 (m, 1H), 8.95-8.80 (m, 1H), 8.75 (dd, J=1.6, 4.8 Hz, 1H), 8.40 (s, 1H), 8.25 (d, J=4.8 Hz, 1H), 8.15 (d, J=8.0 Hz, 1H), 7.80 (s, 1H), 7.75-7.63 (m, 1H), 7.47-7.44 (m, 1H), 5.58 (s, 1H), 4.76-4.56 (m, 1H), 3.51-3.31 (m, 1H), 3.26-3.08 (m, 2H), 3.06-2.91 (m, 2H), 2.04-1.82 (m, 3H). ¹⁹F NMR (376.5 MHz, CDCl₃) δ_{F} −162.750. LCMS purity 98%, MS ESI calcd. for C₂₂H₁₉FN₆O [M+H₂O+H]⁺ 421.2, found 421.2.

### Example 82. Synthesis of 1-((1-([2,4′-bipyridine]-3-carbonyl)-4-fluoropiperidin-4-yl)methyl)-1H-pyrazole-4-carbonitrile (Cmpd 78)

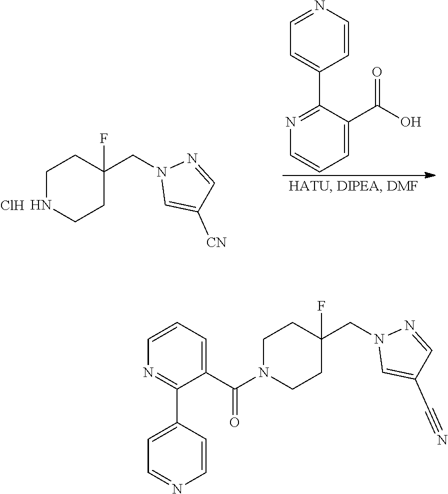 [SMILES: Cl ; N#Cc1cnn(CC2(F)CCNCC2)c1 ; O=C(O)c1cccnc1-c1ccncc1 ; N#Cc1cnn(CC2(F)CCN(C(=O)c3cccnc3-c3ccncc3)CC2)c1]

To a solution of [2-(pyrimidin-4-yl) pyridine-3-carboxylic acid (1.07 g, 5.32 mmol) and HATU (2.53 g, 6.66 mmol) in DMF (30 mL) was added DIPEA (2.3 mL, 13.3 mmol). 1-((4-fluoropiperidin-4-yl)methyl)-1H-pyrazole-4-carbonitrile hydrochloride (330 mg, 1.58 mmol) in DMF (20 mL) was added slowly. The mixture was stirred at 20° C. for 1-2 h. The reaction mixture was poured into H₂O (30 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by Prep-HPLC (Column: Phenomenex C18 80*40 mm*3 um; Condition: water (NH₃H₂O)-ACN; Begin B: 15; End B: 45; Gradient Time (min): 8; 100% B Hold Time (min): 3; FlowRate (ml/min): 30; Injections: 7) provided the product, which was washed with water to afford 1-((1-([2,4′-bipyridine]-3-carbonyl)-4-fluoropiperidin-4-yl)methyl)-1H-pyrazole-4-carbonitrile (50 mg, 35%). Further purification by SFC (Column: Phenomenex C18 80*40 mm*3 um; Condition: water (NH₃H₂O)-ACN; Begin B: 20; End B: 50; Gradient Time (min): 8; 100% B Hold Time (min): 1.3; FlowRate (ml/min): 30; Injections: 6) afforded 1-((1-([2,4′-bipyridine]-3-carbonyl)-4-fluoropiperidin-4-yl)methyl)-1H-pyrazole-4-carbonitrile (36 mg, 74%).

¹H NMR (400 MHz, CDCl₃) δ_{H} 8.85-8.64 (m, 3H), 7.93-7.56 (m, 5H), 7.44 (m, 1H), 4.77-4.50 (m, 1H), 4.39-3.88 (m, 2H), 3.19-2.90 (m, 2H), 2.87-2.50 (m, 1H), 1.84-1.71 (m, 1H), 1.58-1.06 (m, 2H), 0.25-0.05 (m, 1H). ¹⁹F NMR (376.5 MHz, CDCl₃) δ_{F} −165.63. LCMS purity 98.1%, MS ESI calcd. for C₂₁H₁₉FN₆O [M+H]⁺ 391.2, found 391.2.

### Example 83. Synthesis of (4-((5-chloropyrimidin-2-yl)methyl)-4-fluoropiperidin-1-yl)(2-(pyrimidin-4-yl)pyridin-3-yl)methanone (Cmpd 79)

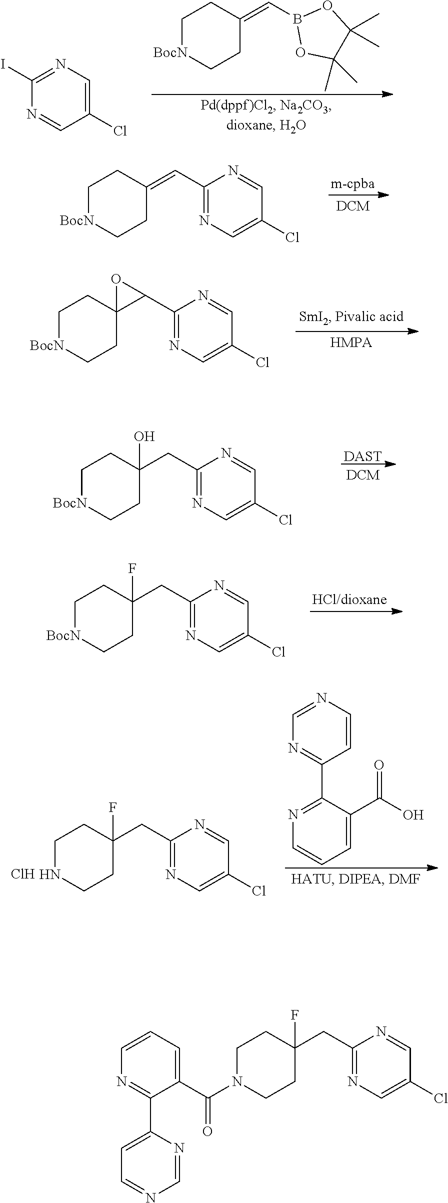

Step 1

A mixture of Pd(dppf)Cl₂ (303 mg, 0.415 mmol), Na₂CO₃ (1.75 g, 16.6 mmol), 5-chloro-2-iodopyrimidine (2 g, 8.31 mmol) and tert-butyl 4-[(4,4,5,5-tetramethyl-1,3,2-dioxaborolan-2-yl)methylidene]piperidine-1-carboxylate (2.68 g, 8.31 mmol) in dioxane (40 mL) and water (8 mL) was stirred at 100° C. for 16 h under N₂. After cooling to 20° C., the mixture was poured into water (100 mL) and extracted with EtOAc (100 mL×2). The combined organic phase was washed with brine (100 mL), dried over Na₂SO₄, filtered, and concentrated. Purification by column (0-20% of EtOAc in PE) provided tert-butyl 4-((5-chloropyrimidin-2-yl)methylene)piperidine-1-carboxylate (2 g).

¹H NMR (400 MHz, CDCl₃) δ_{H} 8.62 (s, 2H), 6.45 (s, 1H), 3.59-3.53 (m, 2H), 3.49 (t, J=6.0 Hz, 2H), 3.09 (t, J=5.6 Hz, 2H), 2.40 (t, J=5.6 Hz, 2H), 1.48 (s, 9H).

Step 2

To a solution of tert-butyl 4-((5-chloropyrimidin-2-yl)methylene)piperidine-1-carboxylate (1.5 g, 4.84 mmol) in DCM (20 mL) was added m-CPBA (4.90 g, 24.2 mmol, 85% purity) at 0° C. and the reaction mixture was stirred at 0° C. for 5 h. The reaction mixture was diluted with Na₂S₂O₃ (50 mL) and NaHCO₃ (50 mL), extracted with DCM (50 ml×2) and the combined organic phase was washed with brine (2×50 mL), dried over anhydrous Na₂SO₄, filtered, and concentrated. Purification by flash column (0-30% of EtOAc in PE) provided tert-butyl 2-(5-chloropyrimidin-2-yl)-1-oxa-6-azaspiro[2.5]octane-6-carboxylate (1.1 g).

¹H NMR (400 MHz, CDCl₃) δ_{H} 8.71 (s, 2H), 4.12 (s, 1H), 3.86-3.74 (m, 1H), 3.63-3.36 (m, 3H), 2.04-1.92 (m, 1H), 1.79-1.65 (m, 3H), 1.45 (s, 9H).

Step 3

To a solution of tert-butyl 2-(5-chloropyrimidin-2-yl)-1-oxa-6-azaspiro[2.5]octane-6-carboxylate (1.1 g, 3.37 mmol) in HMPA (33 mL) was added SmI₂ (84.1 mL, 0.1M in THF, 8.42 mmol) at 20° C. A solution of pivalic acid (27.7 mL, 0.17 M in THF, 4.71 mmol) was added and the solution was allowed to stir for 24 h. The reaction was quenched with an aqueous solution of sodium potassium tartrate (100 mL). The mixture was extracted with EtOAc (3×100 mL) and the organic layer was washed with brine (2×100 mL), dried with Na₂SO₄, and filtered. The solvent was removed in vacuo and the product was purified by flash column (0-40% EtOAc in PE) to afford tert-butyl 4-((5-chloropyrimidin-2-yl)methyl)-4-hydroxypiperidine-1-carboxylate (580 mg).

¹H NMR (400 MHz, CDCl₃) δ_{H} 8.65 (s, 2H), 3.80 (d, J=13.2 Hz, 2H), 3.29-3.19 (m, 2H), 3.13 (s, 2H), 1.58-1.51 (m, 4H), 1.45 (s, 9H).

Step 4

To a mixture of tert-butyl 4-((5-chloropyrimidin-2-yl)methyl)-4-hydroxypiperidine-1-carboxylate (690 mg, 2.10 mmol) in DCM (20 mL) was added DAST (676 mg, 4.20 mmol) at 0° C., the mixture was stirred at 0° C. for 10 min. The mixture was poured into NaHCO₃ (50 mL) and stirred for 10 min. The aqueous phase was extracted with DCM (2×30 mL). The combined organic phase was washed with saturated brine (2×30 mL), dried over anhydrous Na₂SO₄, filtered, and concentrated. Purification by flash column (0-20% EtOAc in PE) and further purification by SFC (Column: DAICEL CHIRALPAK AD (250 mm*30 mm, 10 um); Condition: 0.1% NH₁₁₃H₂O ETOH; Begin B: 15; End B: 15; Flow Rate (ml/min): 70; Injections: 150) provided tert-butyl 4-((5-chloropyrimidin-2-yl)methyl)-4-fluoropiperidine-1-carboxylate (300 mg).

¹H NMR (400 MHz, CDCl₃) δ_{H} 8.66 (s, 2H), 3.93 (d, J=12.0 Hz, 2H), 3.29 (d, J=18.8 Hz, 2H), 3.08 (t, J=11.6 Hz, 2H), 1.92-1.83 (m, 2H), 1.82-1.67 (m, 2H), 1.45 (s, 9H). ¹⁹F NMR (376.5 MHz, CDCl₃) δ_{F} −159.46.

Step 5

To a solution of tert-butyl 4-((5-chloropyrimidin-2-yl)methyl)-4-fluoropiperidine-1-carboxylate (300 mg, 0.9 mmol) in 1,4-dioxane (5 mL) was added hydrogen chloride (10 mL, 40.0 mmol, 4 M in 1,4-dioxane) at 20° C. under N₂ and the reaction mixture was stirred at 20° C. for 1 h. The reaction mixture was concentrated under reduced pressure to give 5-chloro-2-((4-fluoropiperidin-4-yl)methyl)pyrimidine hydrochloride (300 mg).

¹H NMR (400 MHz, DMSO-d₆) δ_{H} 8.92 (s, 2H), 3.31 (d, J=18.8 Hz, 2H), 3.21 (d, J=12.8 Hz, 2H), 3.02-2.88 (m, 2H), 2.14-1.97 (m, 4H). ¹⁹F NMR (376.5 MHz, DMSO-d₆) δ_{F}−155.66.

Step 6

To a solution of 2-(pyrimidin-4-yl)pyridine-3-carboxylic acid (90.5 mg, 0.45 mmol) and HATU (562 mg, 0.112 mmol) in DMF (5 mL) was added DIPEA (0.325 mL, 1.87 mmol, 0.74 g/mL) at 20° C. and stirred for 15 min. 5-chloro-2-((4-fluoropiperidin-4-yl)methyl)pyrimidine hydrochloride (100 mg, 0.375 mmol) in DMF (5 mL) was added slowly at 20° C. The mixture was stirred at 20° C. for 1 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 21, End B: 51; Gradient Time(min): 8; 100% B Hold Time(min): 2.3; FlowRate(ml/min): 30; Injections: 5) provided (4-((5-chloropyrimidin-2-yl)methyl)-4-fluoropiperidin-1-yl)(2-(pyrimidin-4-yl)pyridin-3-yl)methanone (55 mg, 36%).

¹H NMR (400 MHz, CDCl₃) δ_{H} 9.19-8.92 (m, 1H), 8.87 (d, J=4.8 Hz, 1H), 8.74 (dd, J=1.6, 4.8 Hz, 1H), 8.67 (s, 2H), 8.24 (d, J=5.2 Hz, 1H), 7.73-7.62 (m, 1H), 7.45 (dd, J=4.8, 7.6 Hz, 1H), 4.74-4.51 (m, 1H), 3.42 (d, J=7.6 Hz, 1H), 3.33 (d, J=18.8 Hz, 2H), 3.27-3.13 (m, 2H), 2.18-1.90 (m, 2H), 1.89-1.67 (m, 2H). ¹⁹F NMR (376.5 MHz, CDCl₃). δ_{F} −159.34. LCMS purity 99%, MS ESI calcd. For C₂₀H₁₈ClFN₆O [M+H]⁺ 413.2, found 413.2.

### Example 84. Synthesis of 6-((4-fluoro-1-(2-(pyrimidin-4-yl)nicotinoyl)piperidin-4-yl)methyl) picolinonitrile (Cmpd 80)

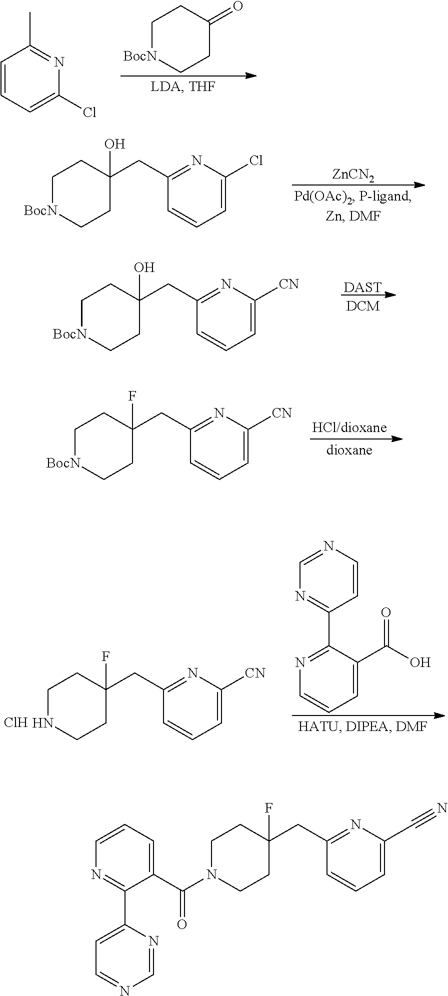

Step 1

To a solution of i-Pr₂NH (6 g, 59.2 mmol) in THE (50 mL) was added n-BuLi (23.6 mL, 2.5 M in hexane, 59.2 mmol) at −70° C. under N₂. The mixture was stirred at −70° C. for 30 mins. To the resulting LDA (6.27 g, 58.6 mmol) solution was added 6-((4-fluoro-1-(2-(pyrimidin-4-yl)nicotinoyl)piperidin-4-yl)methyl)picolinonitrile (5 g, 39.1 mmol) at −70° C. and the mixture was stirred for 1 h. Then tert-butyl 4-oxopiperidine-1-carboxylate (9.34 g, 46.9 mmol) in THE (80 mL) was added. After stirring at −70° C. for 1 h, the mixture was poured into saturated ammonium chloride solution (50 mL) and extracted with EtOAc (50 mL×3). The combined organic phase was washed with brine (2×50 mL), dried over Na₂SO₄, filtered, and concentrated. The residue was purified by flash column (15˜20% of EtOAc in PE) to give tert-butyl 4-((6-chloropyridin-2-yl)methyl)-4-hydroxypiperidine-1-carboxylate (9 g).

¹H NMR (400 MHz, CDCl₃) δ_{H} 7.60 (t, 1H), 7.22 (d, J=8.0 Hz, 1H), 7.06 (d, J=7.2 Hz, 1H), 3.82-3.79 (br m, 2H), 3.20 (m, 2H), 2.89 (s, 2H), 1.49 (q, 4H), 1.45 (s, 9H).

Step 2

A mixture of tert-butyl 4-((6-chloropyridin-2-yl)methyl)-4-hydroxypiperidine-1-carboxylate (2 g, 6.11 mmol), Pd(OAc)₂ (137 mg, 0.611 mmol), {[1,1′-binaphthalen]-2-yl}di-tert-butyl)phosphane (243 mg, 0.611 mmol), zinc (79.7 mg, 1.22 mmol), and Zn(CN)₂ (386 mg, 3.29 mmol) in DMF (20 ml) at 20° C. under N₂ was heated to 110° C. After stirring at 110° C. for 1 h, the reaction was cooled to room temperature and extracted with EtOAc (2×20 mL). The combined organic phase was washed with water (2×20 mL), dried over Na₂SO₄, filtered, and concentrated. Purification by flash column (10-20% of EtOAc in PE) provided tert-butyl 4-((6-cyanopyridin-2-yl)methyl)-4-hydroxypiperidine-1-carboxylate (1.76 g).

¹H NMR (400 MHz, CDCl₃) δ_{H} 7.80 (t, 1H), 7.67-7.55 (d, J=6.8 Hz, 1H), 7.38 (d, J=7.2 Hz, 1H), 4.20 (m, 1H), 3.82 (m, 2H), 3.20 (m, 2H), 1.63 (m, 2H), 1.52 (m, 3H), 1.45 (s, 9H).

Step 3

To a mixture of tert-butyl 4-((6-cyanopyridin-2-yl)methyl)-4-hydroxypiperidine-1-carboxylate (1 g, 3.15 mmol) in DCM (60 mL) was added DAST (609 mg, 3.78 mmol) at 0° C. After stirring at 0° C. for 5 mins, the reaction mixture was added to NaHCO₃ (20 mL) slowly. The aqueous phase was extracted with DCM (3×20 mL). The combined organic phase was washed with brine (2×20 mL), dried over anhydrous Na₂SO₄, filtered, and concentrated. Purification by flash column (5%-10% of EtOAc in PE) and further purification by SFC (Column: DAICEL CHIRALPAK AD (250 mm*30 mm, 10 um); Condition: 0.1% NH₃H₂O ETOH; Begin B: 10%; End B: 10%) provided tert-butyl 4-((6-cyanopyridin-2-yl)methyl)-4-fluoropiperidine-1-carboxylate (225 mg).

¹H NMR (400 MHz, CDCl₃) δ_{H} 7.78 (t, 1H), 7.63-7.57 (d, J=7.6 Hz, 1H), 7.50 (d, J=8.0 Hz, 1H), 3.92 (m, 2H), 3.18 (m, 2H), 3.09-3.00 (m, 2H), 1.61 (m, 2H), 1.45 (s, 9H). ¹⁹F NMR (376.5 MHz, CDCl₃) δ_{F} −160.354.

Step 4

To a mixture of tert-butyl 4-((6-cyanopyridin-2-yl)methyl)-4-fluoropiperidine-1-carboxylate (225 mg, 0.704 mmol) in dioxane (10 mL) was added HCl/dioxane (10 mL, 4M in dioxane, 40.0 mmol). The mixture was stirred at 25° C. for 0.5 h. The reaction mixture was concentrated to give 6-((4-fluoropiperidin-4-yl)methyl)picolinonitrile hydrochloride (225 mg), which was carried directly into the next step.

Step 5

To a solution of [2-(pyrimidin-4-yl)pyridine-3-carboxylic acid (87.9 mg, 0.437 mmol) and HATU (249 mg, 0.656 mmol) in DMF (20 mL) was added DIPEA (0.23 mL, 1.31 mmol) at 20° C. The mixture was stirred for 10-15 mins. 6-((4-fluoropiperidin-4-yl)methyl)picolinonitrile hydrochloride (112.5 mg, 0.438 mmol) in DMF (20 mL) was added slowly and then DIPEA (0.152 mL, 0.867 mmol) was added. After stirring at 20° C. for 2 h, the reaction mixture was poured into H₂O (50 mL) and stirred for 20 mins. The aqueous phase was extracted with EtOAc (3×20 mL). The combined organic phase was washed with brine (2×20 mL), dried over anhydrous Na₂SO₄, filtered, and concentrated. Purification by Prep-HPLC (Column: Phenomenex C18 80*40 mm*3 um; Condition: water (NH₃H₂O)-ACN; Begin B: 22; End B: 52; Gradient Time(min): 8; 100% B Hold Time(min): 2.5; FlowRate (ml/min): 30; Injections: 7) provided 6-((4-fluoro-1-(2-(pyrimidin-4-yl)nicotinoyl)piperidin-4-yl)methyl)picolinonitrile (26.2 mg, 14%).

¹H NMR (400 MHz, CDCl₃) δ_{H} 8.96-8.83 (m, 2H), 8.75 (dd, J=1.6, 4.4 Hz, 1H), 8.25 (m, 1H), 7.79 (t, 1H), 7.62 (m, 2H), 7.48 (br m, 2H), 4.84-4.38 (m, 2H), 3.39 (m, 1H), 3.30-3.11 (m, 5H), 1.95 (m, 2H). ¹⁹F NMR (376 MHz, CDCl₃) δ_{F} −160.248. LCMS purity 98%, MS ESI calcd. for C₂₂H₁₉FN₆O [M+H]⁺ 403.2 found 403.2.

### Example 85. Synthesis of 6-((1-([2,4′-bipyridine]-3-carbonyl)-4-fluoropiperidin-4-yl)methyl)picolinonitrile (Cmpd 81)

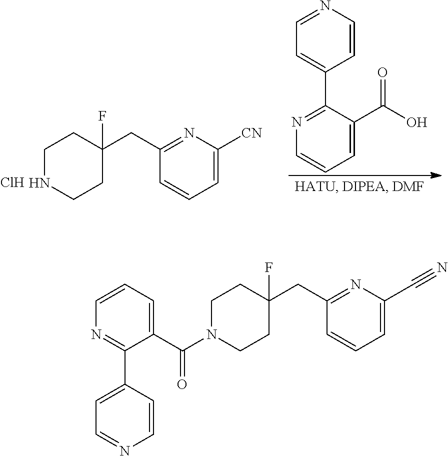 [SMILES: Cl ; N#Cc1cccc(CC2(F)CCNCC2)n1 ; O=C(O)c1cccnc1-c1ccncc1 ; N#Cc1cccc(CC2(F)CCN(C(=O)c3cccnc3-c3ccncc3)CC2)n1]

To a solution of [2-(pyrimidin-4-yl) pyridine-3-carboxylic acid (87.4 mg, 0.437 mmol) and HATU (249 mg, 0.656 mmol) in DMF (20 mL) was added DIPEA (0.23 mL, 1.31 mmol) at 20° C. The mixture was stirred for 15 mins. 6-((4-fluoropiperidin-4-yl)methyl)picolinonitrile hydrochloride (113 mg, 0.44 mmol) in DMF (20 mL) was added slowly and then DIPEA (0.152 mL, 0.867 mmol) was added. After stirring at 20° C. for 2 h, the reaction mixture was poured into H₂O (50 mL) and stirred for 20 mins. The aqueous phase was extracted with EtOAc (3×20 mL). The combined organic phase was washed with brine (2×20 mL), dried over anhydrous Na₂SO₄, filtered, and concentrated. Purification by Prep-HPLC (Column: Phenomenex C18 80*40 mm*3 um; Condition: water (NH₃H₂O)-ACN; Begin B: 22; End B: 52; Gradient Time(min): 8; 100% B Hold Time(min): 2.5; FlowRate (ml/min): 30; Injections: 7) provided 6-((1-([2,4′-bipyridine]-3-carbonyl)-4-fluoropiperidin-4-yl)methyl)picolinonitrile (56 mg, 32%).

¹H NMR (400 MHz, CDCl₃) δ_{H} 8.79 (dd, 1H), 8.65-8.74 (m, 2H), 7.75-7.73 (m, 3H), 7.62-7.60 (m, 2H), 7.40-7.31 (m, 2H), 4.43-4.67 (m, 1H), 2.95-3.20 (m, 3H), 2.90-2.84 (br m, 2H), 2.54-2.65 (m, 1H), 1.90-1.75 (m, 1H), 1.23-1.49 (m, 2H). ¹⁹F NMR (376 MHz, CDCl₃) δ_{F} −161.629. LCMS purity 99%, MS ESI calcd. for C₂₃H₂₀FN₅O [M+H]⁺ 402.2, found 402.2.

### Example 86. Synthesis of (4-((3-chloropyrazin-2-yl)methyl)-4-fluoropiperidin-1-yl)(2-(pyrimidin-4-yl)pyridin-3-yl)methanone (Cmpd 58)

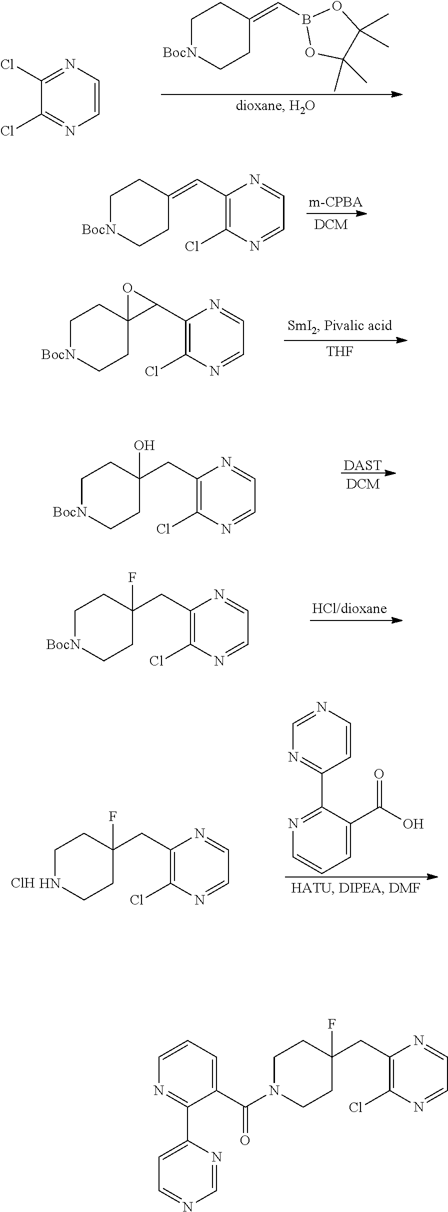

Step 1

A mixture of Pd(dppf)Cl₂ (245 mg, 0.335 mmol), Na₂CO₃ (1.42 g, 13.4 mmol), 2,3-dichloropyrazine (1 g, 6.71 mmol) and tert-butyl 4-[(4,4,5,5-tetramethyl-1,3,2-dioxaborolan-2-yl) methylidene]piperidine-1-carboxylate (1.73 g, 5.36 mmol) in dioxane (20 mL) and water (4 mL) was stirred at 100° C. for 16 hours under N₂. After cooling to 20° C., the mixture was poured into water (50 mL), and the mixture was extracted with EtOAc (50 mL×2). The combined organic phase was washed with water (50 mL) and brine (100 mL), dried over Na₂SO₄, filtered, and concentrated. Purification by flash column (0˜20% EtOAc in PE) provided tert-butyl 4-((3-chloropyrazin-2-yl)methylene)piperidine-1-carboxylate (1 g).

¹H NMR (400 MHz, CDCl₃) δ_{H} 8.44 (m, 1H), 8.17 (m, 1H), 6.59 (s, 1H), 3.57 (m, 2H), 3.49 (m, 2H), 2.88-2.80 (m, 2H), 2.44 (t, J=5.2 Hz, 2H), 1.48 (s, 9H).

Step 2

To a solution of tert-butyl 4-((3-chloropyrazin-2-yl)methylene)piperidine-1-carboxylate (1.2 g, 3.87 mmol) in DCM (50 mL) was added m-CPBA (3.91 g, 19.3 mmol, 85%) at 0° C. and the reaction mixture was stirred at 0° C. for 5 h. The reaction mixture was diluted with Na₂S₂O₃ (100 mL) and stirred 10 min, then extracted with DCM (2×50 ml) and the combined organic phase was dried over Na₂SO₄, filtered, and concentrated. Purification by flash column (0-20% EtOA in PE) provided tert-butyl 2-(3-chloropyrazin-2-yl)-1-oxa-6-azaspiro[2.5]octane-6-carboxylate (1.1 g).

¹H NMR (400 MHz, CDCl₃) δ_{H} 8.55 (m, 1H), 8.34 (m, 1H), 4.19 (s, 1H), 3.82-3.72 (m, 1H), 3.62 (m, 1H), 3.51-3.44 (m, 2H), 2.00 (m, 1H), 1.78 (m, 1H), 1.52-1.48 (m, 2H), 1.46 (s, 9H).

Step 3

To a solution of tert-butyl 2-(3-chloropyrazin-2-yl)-1-oxa-6-azaspiro[2.5]octane-6-carboxylate (200 mg, 0.613 mmol) in HMPA (6 mL) at 20° C. was added SmI₂ (18.3 mL, 0.1M in THF, 1.83 mmol). A solution of pivalic acid (5.40 mL, 0.17 M in THF, 0.919 mmol) was added and the solution was allowed to stir for 48 h. The reaction was quenched with an aqueous solution of sodium potassium tartrate (40 mL). The mixture was extracted with EtOAc (3×20 mL) and the organic layer was washed with H₂O (2×20 mL) and dried over Na₂SO₄. The solvent was removed in vacuo and the crude product was purified by flash column (0-40% EtOAc in PE) to afford tert-butyl 4-((3-chloropyrazin-2-yl)methyl)-4-hydroxypiperidine-1-carboxylate (120 mg).

¹H NMR (400 MHz, CDCl₃) δ_{H} 8.56 (m, 1H), 8.34 (m, 1H), 4.19 (s, 1H), 3.89-3.72 (m, 2H), 3.50-3.45 (m, 2H), 2.00 (m, 1H), 1.78 (m, 1H), 1.58-1.55 (m, 2H), 1.46 (s, 9H).

Step 4

To a mixture of tert-butyl 4-((3-chloropyrazin-2-yl)methyl)-4-hydroxypiperidine-1-carboxylate (120 mg, 0.366 mmol) in DCM (10 mL) was added DAST (117 mg, 0.732 mmol) at 0° C. The mixture was stirred at 0° C. for 30 min. The mixture was poured into ice-water (10 mL) and NaHCO₃ (10 mL) and stirred for 10 min. The aqueous phase was extracted with DCM (2×20 mL). The combined organic phase was washed with saturated brine (2×20 mL), dried over anhydrous Na₂SO₄, filtered, and concentrated. Purification by flash column (0-50% EtOAc in PE) and further purification by SFC (Column: DAICEL CHTRALCEL OD-H (250 mm*30 mm, 5 um); Condition: 0.1% NH₃H₂O ETOH; Begin B: 15%; End B; 15%; Flow Rate (ml/min): 60; Injections: 50) provided tert-butyl 4-((3-chloropyrazin-2-yl)methyl)-4-fluoropiperidine-1-carboxylate (10 mg, 8%).

¹H NMR (400 MHz, CDCl₃) δ_{H} 8.48 (m, 1H), 8.28 (m, 1H), 3.96 (m, 2H), 3.35 (d, J=18.4 Hz, 2H), 3.05 (t, J=12.0 Hz, 2H), 1.89-1.73 (m, 4H), 1.45 (s, 9H). ¹⁹F NMR (376.5 MHz, CDCl₃) δ^{F} −159.686.

Step 5

To a solution of tert-butyl 4-((3-chloropyrazin-2-yl)methyl)-4-fluoropiperidine-1-carboxylate (20 mg, 0.0606 mmol) in 1,4-dioxane (5 mL) was added hydrogen chloride (5 mL, 20.0 mmol, 4 M in 1,4-dioxane) at 20° C. under N₂ and the reaction mixture was stirred at 20° C. for 2 h. The reaction mixture was concentrated under reduced pressure to give 2-chloro-3-((4-fluoropiperidin-4-yl)methyl)pyrazine hydrochloride (20 mg), which was carried directly into the next step.

Step 6

To a solution of 2-(pyrimidin-4-yl)pyridine-3-carboxylic acid (18.1 mg, 0.0901 mmol) and HATU (42.5 mg, 0.112 mmol) in DMF (2 mL) was added DIPEA (0.0654 mL, 0.375 mmol, 0.74 g/mL) at 20° C. 2-chloro-3-((4-fluoropiperidin-4-yl)methyl)pyrazine hydrochloride (20 mg, 0.0751 mmol) in DMF (2 mL) was added slowly at 20° C. and the mixture was stirred for 1 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 25; End B: 55; Gradient Time(min): 8; 100% B Hold Time(min): 2; Flow Rate (ml/min): 30; Injections 4) provided (4-((3-chloropyrazin-2-yl)methyl)-4-fluoropiperidin-1-yl)(2-(pyrimidin-4-yl)pyridin-3-yl)methanone (6.9 mg, 22%).

¹H NMR (400 MHz, CDCl₃) δ_{H} 9.21-8.83 (m, 2H), 8.75 (d, J=2.4 Hz, 1H), 8.50 (s, 1H), 8.34-8.23 (m, 2H), 7.73-7.64 (m, 1H), 7.49-7.43 (m, 1H), 4.78-4.56 (m, 1H), 3.48-3.36 (m, 3H), 3.29-3.10 (m, 2H), 2.20-1.97 (m, 2H), 1.96-1.70 (m, 2H). ¹⁹F NMR (376.5 MHz, CDCl₃). δ_{F} −159.078. LCMS purity 99%, MS ESI calcd. For C₂₀H₁₈ClFN₆ONa [M+Na]⁺ 435.1, found 435.1.

### Example 87. Synthesis of [2,4′-bipyridin]-3-yl(4-((3-chloropyrazin-2-yl)methyl)-4-fluoropiperidin-1-yl)methanone (Cmpd 59)

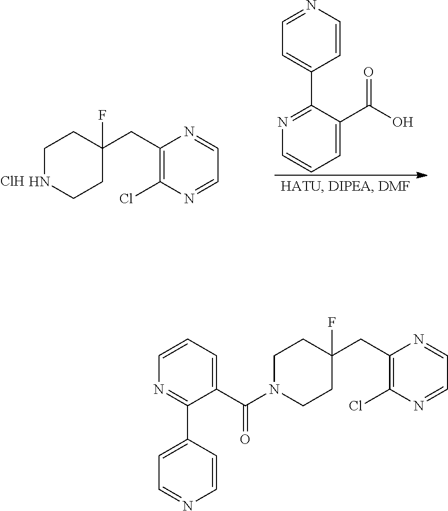 [SMILES: Cl ; FC1(Cc2nccnc2Cl)CCNCC1 ; O=C(O)c1cccnc1-c1ccncc1 ; O=C(c1cccnc1-c1ccncc1)N1CCC(F)(Cc2nccnc2Cl)CC1]

To a solution of [2,4′-bipyridine]-3-carboxylic acid (9.00 mg, 0.045 mmol) and HATU (21.3 mg, 0.0562 mmol) in DMF (2 mL) was added DIPEA (0.0325 mL, 0.187 mmol). 2-chloro-3-((4-fluoropiperidin-4-yl)methyl)pyrazine hydrochloride (10 mg, 0.0375 mmol) in DMF (2 mL) was added slowly. The mixture was stirred at 20° C. for 1 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 18; End B: 48; Gradient Time(min): 8; 100% B Hold Time(min): 2; Flow Rate (ml/min): 30; Injections: 3) provided [2,4′-bipyridin]-3-yl(4-((3-chloropyrazin-2-yl)methyl)-4-fluoropiperidin-1-yl)methanone (3 mg, 19%).

¹H NMR (400 MHz, CDCl₃) S_{H} 8.80 (d, J=4.8 Hz, 1H), 8.68 (s, 2H), 8.47 (s, 1H), 8.31 (s, 1H), 7.77 (d, J=5.6 Hz, 3H), 7.45 (dd, J=4.8, 7.6 Hz, 1H), 4.68-4.58 (m, 1H), 3.42-3.13 (m, 2H), 3.12-2.86 (m, 3H), 2.06-1.82 (m, 2H), 1.73-1.58 (m, 2H). ¹⁹F NMR (376.5 MHz, CDCl₃). δ_{F} −159.990. LCMS purity 99%, MS ESI calcd. For C₂₁H₁₉ClFN₅O [M+H]⁺ 412.1, found 412.1.

### Example 88. Synthesis of (4-fluoro-4-(imidazo[1,2-a]pyridin-6-ylmethyl)piperidin-1-yl)(2-(pyrimidin-4-yl)pyridin-3-yl)methanone (Cmpd 82)

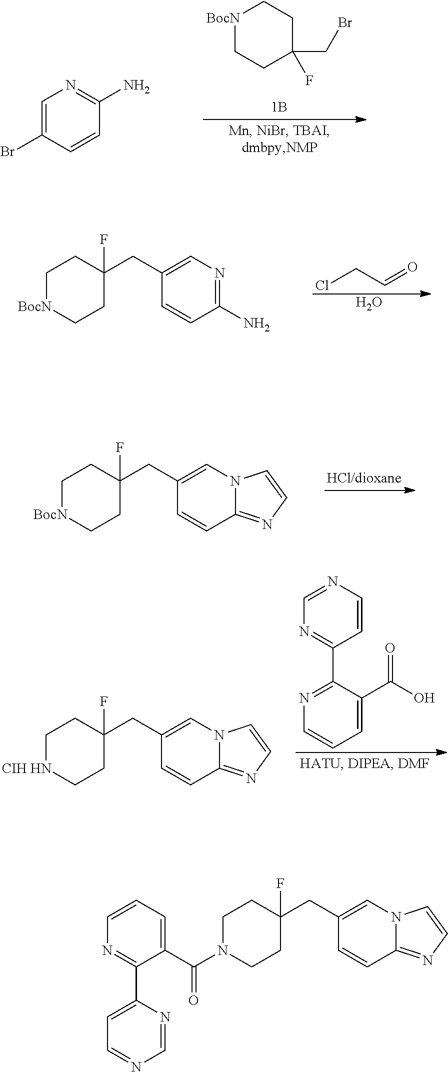

Step 1

To a solution 5-bromopyridin-2-amine (1 g, 5.77 mmol) and tert-butyl 4-(bromomethyl)-4-fluoropiperidine-1-carboxylate (2.04 g, 6.92 mmol) in NMP (10 mL) was added NiBr₂ (251 mg, 1.15 mmol), Mn (1.26 g, 23.0 mmol), MgCl₂ (823 mg, 8.65 mmol) and dmbpy (211 mg, 1.15 mmol) in one portion at 20° C. under N₂. Then the mixture was stirred at 80° C. for 16 h. The mixture was filtered and the filtrate was poured into NH₄Cl (50 mL) and extracted with EtOAc (3×50 mL). The combined organic phase was washed with brine (100 mL), dried over anhydrous Na₂SO₄, filtered, and concentrated. Purification by HPLC (Column: Phenomenex C18 80*40 mm*3 um; Condition: water (NH₃H₂O)-ACN; Begin B: 31; End B: 61; Gradient Time(min): 8; 100% B Hold Time(min): 2; Flow Rate (ml/min): 30; Injections: 8) to give tert-butyl 4-((6-aminopyridin-3-yl)methyl)-4-fluoropiperidine-1-carboxylate (500 mg).

¹H NMR (400 MHz, CDCl₃) δ_{H} 7.86 (d, J=2.0 Hz, 1H), 7.31 (dd, J=1.2, 8.4 Hz, 1H), 6.47 (d, J=8.4 Hz, 1H), 4.38 (s, 2H), 3.92 (d, J=4.4 Hz, 2H), 3.02 (t, J=11.6 Hz, 2H), 2.80-2.69 (m, 2H), 1.75-1.67 (m, 2H), 1.61-1.50 (m, 2H), 1.44 (s, 9H). ¹⁹F NMR (376.5 MHz, CDCl₃) δ_{F} −162.30.

Step 2

A solution of tert-butyl 4-((6-aminopyridin-3-yl)methyl)-4-fluoropiperidine-1-carboxylate (500 mg, 1.61 mmol) and 2-chloroacetaldehyde (787 mg, 40% in H₂O, 4.02 mmol) in H₂O (10 mL) under N₂ was stirred at 20° C. for 10 min and then heated to 80° C. for 12 h. The mixture was cooled and poured into Na₂CO₃ (20 mL) to obtain a mixture with a pH of 8. The aqueous phase was extracted with DCM (3×20 mL). The combined organic phase was washed with saturated brine (2×50 mL), dried over anhydrous Na₂SO₄, filtered, and concentrated to give tert-butyl 4-fluoro-4-(imidazo[1,2-a]pyridin-6-ylmethyl)piperidine-1-carboxylate (600 mg).

¹H NMR (400 MHz, CDCl₃) δ_{H} 7.98 (s, 1H), 7.62 (d, J=1.2 Hz, 1H), 7.58-7.53 (m, 2H), 7.05 (d, J=9.2 Hz, 1H), 4.04-3.84 (m, 2H), 3.11-2.97 (m, 2H), 2.92-2.82 (m, 2H), 1.75 (d, J=10.4 Hz, 2H), 1.55 (s, 2H), 1.45 (s, 9H). ¹⁹F NMR (376.5 MHz, CDCl₃) δ_{F} −162.145.

Step 3

To a solution of tert-butyl 4-fluoro-4-(imidazo[1,2-a]pyridin-6-ylmethyl)piperidine-1-carboxylate (600 mg, 1.79 mmol) in 1,4-dioxane (5 mL) was added hydrogen chloride (10 mL, 40.0 mmol, 4 M in 1,4-dioxane) under N₂, and the reaction mixture was stirred at 20° C. for 1 h. The reaction mixture was concentrated under reduced pressure to give 6-((4-fluoropiperidin-4-yl)methyl)imidazo[1,2-a]pyridine hydrochloride (500 mg), which was carried directly into the next step.

Step 4

To a solution of 2-(pyrimidin-4-yl)pyridine-3-carboxylic acid (80.2 mg, 0.399 mmol) and HATU (189 mg, 0.499 mmol) in DMF (5 mL) was added DIPEA (0.289 mL, 1.66 mmol, 0.74 g/mL) at 20° C. 6-((4-fluoropiperidin-4-yl)methyl)imidazo[1,2-a]pyridine hydrochloride (90 mg, 0.333 mmol) in DMF (5 mL) was added slowly at 20° C. The mixture was stirred at 20° C. for 16 h. The reaction mixture was poured into H₂O (10 mL) and stirred for 20 min. The aqueous phase was extracted with EtOAc (3×10 mL). The combined organic phase was washed with saturated brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 20; End B: 50; Gradient Time(min): 8; 100% B Hold Time(min): 2; FlowRate(ml/min): 30; Injections: 4) provided (4-fluoro-4-(imidazo[1,2-a]pyridin-6-ylmethyl)piperidin-1-yl)(2-(pyrimidin-4-yl)pyridin-3-yl)methanone (34 mg, 24%).

¹H NMR (400 MHz, CDCl₃) δ_{H} 9.19-9.04 (m, 1H), 8.92-8.83 (m, 1H), 8.75 (dd, J=1.6, 4.8 Hz, 1H), 8.25 (d, J=4.0 Hz, 1H), 7.98 (s, 1H), 7.73-7.65 (m, 1H), 7.62 (d, J=1.2 Hz, 1H), 7.58-7.52 (m, 2H), 7.45 (dd, J=4.8, 7.6 Hz, 1H), 7.03 (d, J=9.2 Hz, 1H), 4.76-4.56 (m, 1H), 3.49-3.34 (m, 1H), 3.25-3.06 (m, 2H), 2.99-2.79 (m, 2H), 2.09-1.94 (m, 1H), 1.92-1.69 (m, 2H), 1.59-1.45 (m, 1H). ¹⁹F NMR (376.5 MHz, CDCl₃). δ_{F} −162.615. LC-ELSD/MS purity 97%, MS ESI calcd. For C₂₃H₂₁FN₆O [M+H]⁺ 417.2, found 417.2.

### Example 89. Synthesis of [2,4′-bipyridin]-3-yl(4-fluoro-4-(imidazo[1,2-a]pyridin-6-ylmethyl)piperidin-1-yl)methanone (Cmpd 84)

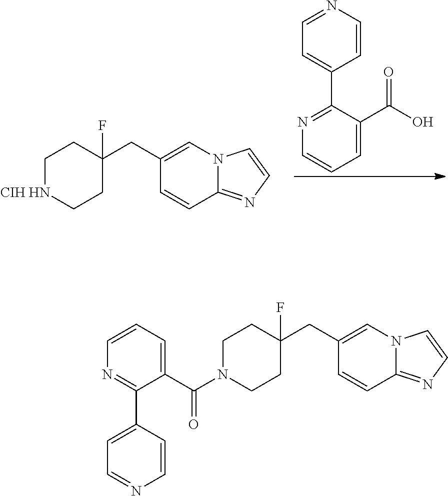

To a solution of [2,4′-bipyridine]-3-carboxylic acid (177 mg, 0.889 mmol) and HATU (422 mg, 1.11 mmol) in DMF (5 mL) was added DIPEA (0.645 mL, 3.70 mmol) at 20° C. 6-((4-fluoropiperidin-4-yl)methyl)imidazo[1,2-a]pyridine hydrochloride (200 mg, 0.741 mmol) in DMF (5 mL) was added slowly into the mixture. The mixture was stirred at 20° C. for 16 h. The reaction mixture was poured into H₂O (10 mL) and stirred for 20 min. The aqueous phase was extracted with EtOAc (3×10 mL). The combined organic phase was washed with brine (2×10 mL), dried over anhydrous Na₂SO₄, filtered, and concentrated. Purification by HPLC (Column: Phenomenex C18 80*40 mm*3 um; Condition: water (NH₃H₂O)-ACN; Begin B: 20; End B: 50; Gradient Time(min): 8; 100% B Hold Time(min): 2; FlowRate(ml/min): 30; Injections: 6) provided [2,4′-bipyridin]-3-yl(4-fluoro-4-(imidazo[1,2-a]pyridin-6-ylmethyl)piperidin-1-yl)methanone (39 mg, 16%).

¹H NMR (400 MHz, CDCl₃) δ_{H} 8.81-8.74 (m, 2H), 8.72-8.65 (m, 1H), 7.92-7.85 (m, 1H), 7.81-7.74 (m, 2H), 7.61 (s, 1H), 7.57-7.49 (m, 2H), 7.46-7.40 (m, 2H), 6.99-6.86 (m, 1H), 4.71-4.53 (m, 1H), 3.09-2.99 (m, 1H), 2.98-2.89 (m, 1H), 2.88-2.70 (m, 1H), 2.69-2.56 (m, 1H), 2.54-2.39 (m, 1H), 1.92-1.77 (m, 1H), 1.46-1.14 (m, 2H), −0.03-0.23 (m, 1H). ¹⁹F NMR (376.5 MHz, CDCl₃). δ_{F} −163.853. LCMS purity 95%, MS ESI calcd. For C₂₄H₂₂FN₅O [M+H]⁺ 416.0, found 416.0.

### Example 90. Synthesis of (4-((5-chloropyridin-2-yl)methyl)-4-fluoropiperidin-1-yl)(3-(pyridin-4-yl)pyridazin-4-yl)methanone (Cmpd 83)

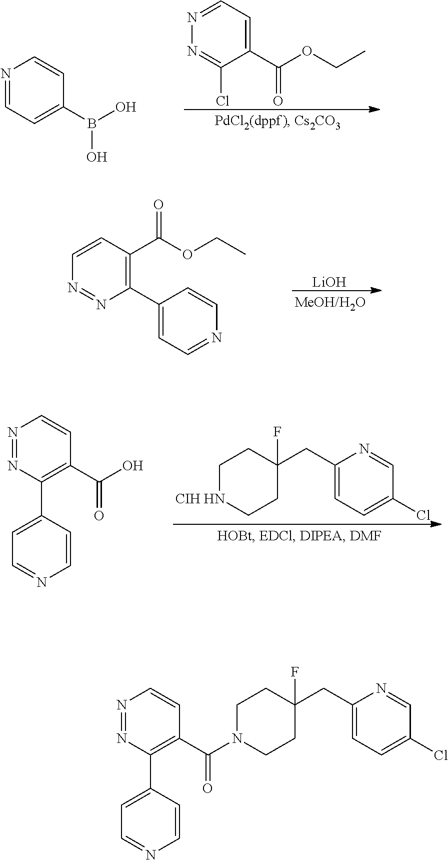

Step 1

To a solution of (pyridin-4-yl)boronic acid (400 mg, 3.25 mmol) in dioxane (20 mL) under N₂ at 20° C. was added PdCl₂(dppf) (47.6 mg, 0.065 mmol), ethyl 3-chloropyridazine-4-carboxylate (544 mg, 2.92 mmol) and Cs₂CO₃ (2.11 g, 6.50 mmol). The mixture was stirred at 20° C. for 0.5 h and 75° C. for 12 h. The mixture was poured into 10% NH₄Cl (100 mL) and extracted with EtOAc (2×50 mL). The combined organic layers were washed with brine (100 mL), dried over Na₂SO₄, filtered, and concentrated. Purification by combi-flash (10-50% of EtOAc in PE) provided ethyl 3-(pyridin-4-yl)pyridazine-4-carboxylate (210 mg, 28%).

¹H NMR (400 MHz, CDCl₃) δ_{H} 9.44 (m, 1H) 8.87-8.70 (m, 2H), 7.89 (d, J=6.0 Hz, 1H), 7.60-7.50 (m, 2H), 4.26 (m, 2H), 1.15 (t, J=8.0 Hz, 3H).

Step 2

To a mixture of ethyl 3-(pyridin-4-yl)pyridazine-4-carboxylate (210 mg, 0.916 mmol) in MeOH (5 mL) and H₂O (1 mL) was added LiOH H₂O (42.0 mg, 1.00 mmol) and the mixture was stirred at 60° C. for 16 h. The reaction mixture was concentrated to dryness and then triturated from DCM (20 mL) at 20° C. for 1 h and filtered. The filter cake was washed with DCM (20 mL) and dried in vacuum to give 3-(pyridin-4-yl)pyridazine-4-carboxylic acid (200 mg).

¹H NMR (400 MHz, CD₃OD) δ_{H} 9.22 (d, J=6.0 Hz, 1H), 8.73-8.63 (m, 2H), 7.93-7.84 (m, 2H), 7.74 (d, J=8.0 Hz, 1H).

Step 3

To a solution of 3-(pyridin-4-yl)pyrazine-2-carboxylic acid (100 mg, 0.497 mmol), EDCI HCl (114 mg, 0.596 mmol), and HOBt (80.5 mg, 0.596 mmol) in DMF (5 mL) was added DIPEA (0.259 mL, 1.49 mmol). 5-chloro-2-[(4-fluoropiperidin-4-yl)methyl]pyridine hydrochloride (131 mg, 0.497 mmol) in DMF (5 mL) was added slowly. The mixture was stirred at 20° C. for 12 h. The reaction mixture was poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by Prep-HPLC (Column: Welch Xtimate C18 100*40 mm*3 um; Condition: water (TFA)-ACN; Begin B: 5; End B: 35; Gradient Time(min): 8; 100% B Hold Time(min): 2; FlowRate(ml/min): 60; Injections: 2) followed by further purification by Prep-HPLC (Column: Phenomenex C18 80*40 mm*3 um; Condition: water (NH₃H₂O)-ACN; Begin B: 22; End B: 52; Gradient Time (min): 8; 100% B Hold Time (min): 1; FlowRate(ml/min): 30; Injections: 2) provided (4-((5-chloropyridin-2-yl)methyl)-4-fluoropiperidin-1-yl)(3-(pyridin-4-yl)pyridazin-4-yl)methanone (8.2 mg, 27%).

¹H NMR (400 MHz, CDCl₃) δ_{H} 9.34-9.32 (m, 1H), 8.77-8.76 (m, 2H), 8.47-8.43 (m, 1H), 7.83-7.67 (m, 2H), 7.59-7.56 (m, 1H), 7.52 (s, 1H), 7.19-7.01 (m, 1H), 4.53 (s, 1H), 3.18-2.94 (m, 2H), 2.91-2.78 (m, 2H), 1.82-1.80 (m, 2H), 1.54-1.33 (m, 2H), 0.39-0.13 (m, 1H). ¹⁹F NMR (376.5 MHz, CDCl₃) δ_{F} −160.48. LCMS purity 97%, MS ESI calcd. for C₂₁H₁₉ClFN₅O [M+H]⁺ 412.1, found 412.1.

### Example 91. Synthesis of [4,4′-bipyrimidin]-5-yl(4-((5-chloropyridin-2-yl)methyl)-4-fluoropiperidin-1-yl)methanone (Cmpd 87)

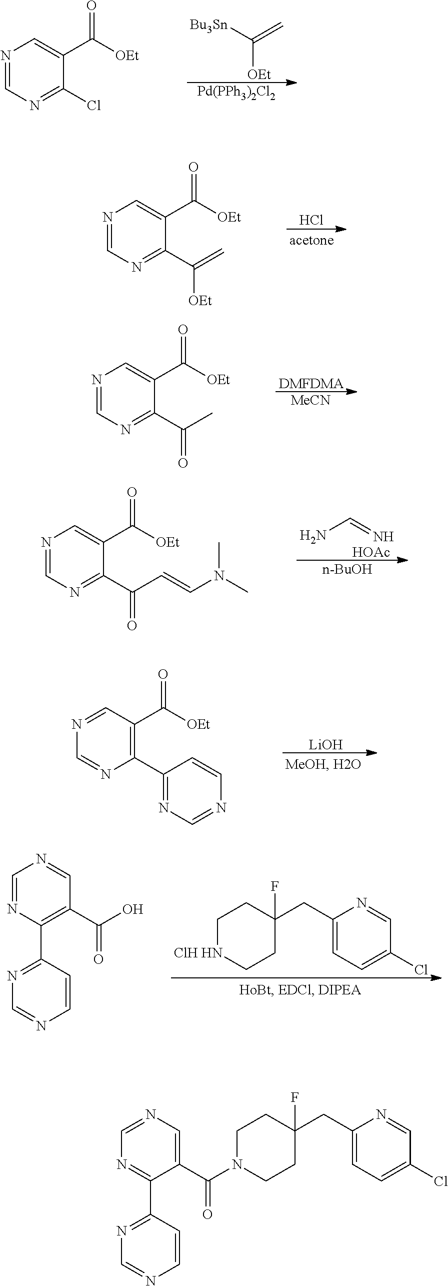

Step 1

To a solution of ethyl 4-chloropyrimidine-5-carboxylate (5 g, 26.7 mmol) in DMF (50 mL) was added tributyl(1-ethoxyvinyl)stannane (11.5 g, 32.0 mmol) at 20° C. under N₂ and the mixture was stirred for 15 min. Then Pd(PPh₃)₂Cl₂ (1.87 g, 2.67 mmol) was added and the mixture was stirred for another 5 min at 20° C. The reaction mixture was heated to 100° C. for 16 h. The residue was poured into aq. KF (10 g in 200 mL water). The reaction mixture was diluted with water (100 mL) and extracted with EtOAc (2×100 mL). The combined organic extracts were dried over anhydrous Na₂SO₄, filtered and concentrated to afford ethyl 4-(1-ethoxyvinyl)pyrimidine-5-carboxylate (5 g), which was used directly in the next step without further purification

Step 2

To a solution of ethyl 4-(1-ethoxyvinyl)pyrimidine-5-carboxylate (5 g, 22.4 mmol) in acetone (150 mL) was added HCl (53.6 mL, 134 mmol, 2.5 M in water) and the mixture was stirred at 20° C. for 2 h. The volatiles were removed under reduced pressure. The mixture was poured into NaHCO₃ (200 mL, aq.) The reaction mixture was extracted with DCM (3×100 mL). The combined organic layers were washed with brine (300 mL), dried over Na₂SO₄, filtered, and concentrated. Purification by column (0-30% of EtOAc in PE) provided ethyl 4-acetylpyrimidine-5-carboxylate (300 mg).

¹H NMR (400 MHz, CDCl₃) δ_{H} 9.34 (s, 1H), 9.16 (s, 1H), 4.42 (q, J=7.2 Hz, 2H), 2.67 (s, 3H), 1.39 (t, J=7.2 Hz, 3H).

Step 3

To a solution of ethyl 4-acetylpyrimidine-5-carboxylate (300 mg, 1.35 mmol) in MeCN (3 mL) was added DMF-DMA (0.3 ml, 2.27 mmol, 0.904 g/mL) at 20° C. Then the mixture was stirred at 80° C. for 3 h. The reaction mixture was concentrated to give ethyl (E)-4-(3-(dimethylamino)acryloyl)pyrimidine-5-carboxylate (400 mg) which was used directly in the next step.

Step 4

To a mixture of ethyl (E)-4-(3-(dimethylamino)acryloyl)pyrimidine-5-carboxylate (400 mg, 1.60 mmol) in n-BuOH (4 mL) was added acetic acid, methanimidamide (2.08 g, 20.0 mmol), and DIPEA (2.89 g, 22.4 mmol) at 25° C., and the mixture was stirred at 120° C. for 16 h. The mixture was poured into water (10 mL) and saturated NaHCO₃ (10 mL). The aqueous phase was extracted with EtOAc (3×10 mL) and the combined organic extracts were washed with brine (2×20 mL), dried over anhydrous Na₂SO₄, filtered, and concentrated. The residue was purified by flash column (0˜30% EtOAc in PE) to give ethyl [4,4′-bipyrimidine]-5-carboxylate (150 mg).

¹H NMR (400 MHz, CDCl₃) δ_{H} 9.39 (s, 1H), 9.28 (d, J=1.2 Hz, 1H), 9.06 (s, 1H), 8.99 (d, J=5.2 Hz, 1H), 8.17 (dd, J=1.6, 5.2 Hz, 1H), 4.37 (q, J=7.2 Hz, 2H), 1.27-1.25 (m, 3H).

Step 5

To a solution of ethyl [4,4′-bipyrimidine]-5-carboxylate (150 mg, 0.651 mmol) in MeOH (3 mL) and H₂O (0.3 mL) was added LiOH·H₂O (32.7 mg, 0.781 mmol) at 25° C. The mixture was stirred at 60° C. for 2 h. The reaction mixture was concentrated to give [4,4′-bipyrimidine]-5-carboxylic acid (150 mg).

¹H NMR (400 MHz, MeOH) δ_{H} 9.25 (s, 1H), 9.21 (s, 1H), 8.96 (s, 1H), 8.94 (d, J=5.2 Hz, 1H), 8.14-8.11 (m, 1H).

Step 6

To a solution of [4,4′-bipyrimidine]-5-carboxylic acid (100 mg, 0.497 mmol), EDCI HCl (113 mg, 0.592 mmol), and HOBt (79.9 mg, 0.592 mmol) in DMF (5 mL) was added DIPEA (0.258 mL, 1.48 mmol) at 20° C. 5-chloro-2-[(4-fluoropiperidin-4-yl)methyl]pyridine hydrochloride (130 mg, 0.494 mmol) in DMF (5 mL) was added slowly into the mixture. The mixture was stirred at 20° C. for 15 h. The reaction mixture was poured into H₂O (30 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by Prep-HPLC (Column: Phenomenex C18 80*40 mm*3 um; Condition: water (NH₃H₂O)-ACN; Begin B: 25; End B: 55; Gradient Time(min): 8; 100% B Hold Time(min): 2; FlowRate(ml/min): 30; Injections: 5) and further purification by SFC (Column: DAICEL CHIRALCEL OD-H (250 mm*30 mm, 5 um); Condition: 0.1% NH₃H₂O ETOH; Begin B: 25%; End B: 25%; FlowRate(ml/min): 70; Injections: 40) provided [4,4′-bipyrimidin]-5-yl(4-((5-chloropyridin-2-yl)methyl)-4-fluoropiperidin-1-yl)methanone (26 mg, 44%).

¹H NMR (400 MHz, CDCl₃) δ_{H} 9.34-9.20 (m, 1H), 8.97 (d, J=4.4 Hz, 2H), 8.75 (m, 1H), 8.49 (d, J=2.4 Hz, 1H), 8.32 (d, J=2.0 Hz, 1H), 7.63 (dd, J=2.4, 8.4 Hz, 1H), 7.22 (d, J=8.4 Hz, 1H), 4.72-4.51 (m, 1H), 3.55-3.36 (m, 1H), 3.21 (s, 2H), 3.18 (d, J=2.4 Hz, 1H), 3.12 (d, J=3.6 Hz, 1H), 2.09-1.92 (m, 2H), 1.91-1.65 (m, 2H). ¹⁹F NMR (376.5 MHz, CDCl₃). δ_{F} −160.063. LCMS purity 96%, MS ESI calcd. For C₂₀H₁₈ClFN₆O [M+H]⁺ 413.1, found 413.1.

### Example 92. Synthesis of (4-((5-chloropyridin-2-yl)methyl)-4-fluoropiperidin-1-yl)(4-(pyridin-4-yl)pyrimidin-5-yl)methanone (Cmpd 88)

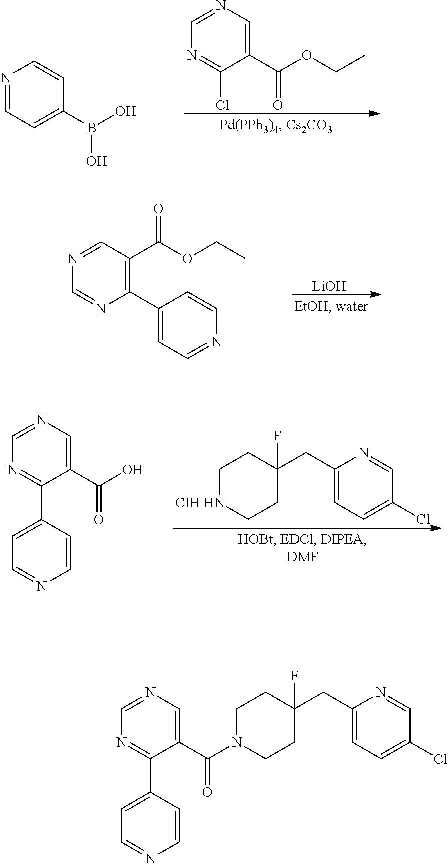

Step 1

To a solution of (pyridin-4-yl)boronic acid (800 mg, 6.50 mmol) in dioxane (20 mL) under N₂ at 20° C. was added PdCl₂(dppf) (95.3 mg, 0.13 mmol), ethyl 4-chloropyrimidine-5-carboxylate (1.09 g, 5.85 mmol) and Cs₂CO₃ (4.23 g, 13.0 mmol). The mixture was stirred at 20° C. for 0.5 h and 75° C. for 12 h. The mixture was poured into 10% NH₄Cl (100 mL) and extracted with EtOAc (2×50 mL). The combined organic layers were washed with brine (100 mL), dried over Na₂SO₄, filtered, and concentrated. Purification by combi-flash (10-50% of EtOAc in PE) provided ethyl 4-(pyridin-4-yl)pyrimidine-5-carboxylate (230 mg).

¹H NMR (400 MHz, CDCl₃) δ_{H} 9.39 (s, 1H), 9.23 (s, 1H), 8.90 (s, 1H), 8.82-8.72 (m, 2H), 8.62 (s, 1H), 7.56-7.43 (m, 2H), 4.28 (m, 2H), 1.18 (m, 3H).

Step 2

To a mixture of ethyl 4-(pyridin-4-yl)pyrimidine-5-carboxylate (220 mg, 0.9596 mmol) in MeOH (5 mL)/H₂O (1 mL) was added LiOH H₂O (44.1 mg, 1.05 mmol) and the mixture was stirred at 60° C. for 16 h. The reaction mixture was concentrated to dryness and then triturated from DCM (20 mL) at 20° C. for 1 h and filtered. The filter cake was washed with DCM (20 mL) and dried in vacuum to give 4-(pyridin-4-yl)pyrimidine-5-carboxylic acid (200 mg).

¹H NMR (400 MHz, CD₃OD) δ_{H} 9.18 (s, 1H), 8.90 (s, 1H), 8.68-8.63 (m, 2H), 7.90-7.86 (m, 2H).

Step 3

To a solution of 4-(pyridin-4-yl)pyrimidine-5-carboxylic acid (100 mg, 0.497 mmol), EDCI HCl (114 mg, 0.596 mmol), and HOBt (80.5 mg, 0.596 mmol) in DMF (5 mL) was added DIPEA (0.259 mL, 1.49 mmol). 5-chloro-2-[(4-fluoropiperidin-4-yl)methyl]pyridine hydrochloride (131 mg, 0.497 mmol) in DMF (5 mL) was added slowly. The mixture was stirred at 20° C. for 12 h. The reaction was mixture poured into H₂O (50 mL) and stirred for 20 min. The aqueous phase was extracted with EtOAc (3×20 mL). The combined organic phase was washed with saturated brine (2×20 mL), dried over anhydrous Na₂SO₄, filtered, and concentrated. Purification by flash Prep-HPLC (Column Phenomenex C18 80*40 mm*3 um Condition water (NH₃H₂O)-ACN Begin B 24End B 54 Gradient Time(min) 8100% B Hold Time(min) 2 FlowRate(ml/min) 30Injections 4) and further purification by SFC (Column DAICEL CHIRALPAK AD (250 mm*30 mm, 10 um) Condition 0.1% NH₃H₂O IPA Begin B 35% End B 35% Gradient Time(min) 100% B Hold Time(min) FlowRate(ml/min) 70 Injections 60) provided (4-((5-chloropyridin-2-yl)methyl)-4-fluoropiperidin-1-yl)(4-(pyridin-4-yl)pyrimidin-5-yl)methanone (24 mg, 69%).

¹H NMR (400 MHz, CDCl₃) δ_{H} 9.35 (s, 1H), 8.84-8.72 (m, 3H), 8.47 (s, 1H), 7.78-7.63 (m, 2H), 7.60-7.58 (m, 1H), 7.20-7.03 (m, 1H), 4.58-4.55 (m, 1H), 3.24-3.07 (m, 1H), 3.06-2.64 (m, 4H), 1.91-1.78 (m, 1H), 1.58-1.32 (m, 3H). ¹⁹F NMR (376.5 MHz, CDCl₃) δ_{F} −160.35. LCMS purity 99%; MS ESI calcd. for C₂₁H₁₉ClFN₅O [M+H]⁺ 412.1, found 412.1.

### Example 93. Synthesis of (4-((5-chloropyridin-2-yl)methyl)-4-fluoropiperidin-1-yl)(3-(pyrimidin-4-yl)pyrazin-2-yl)methanone (Cmpd 89)

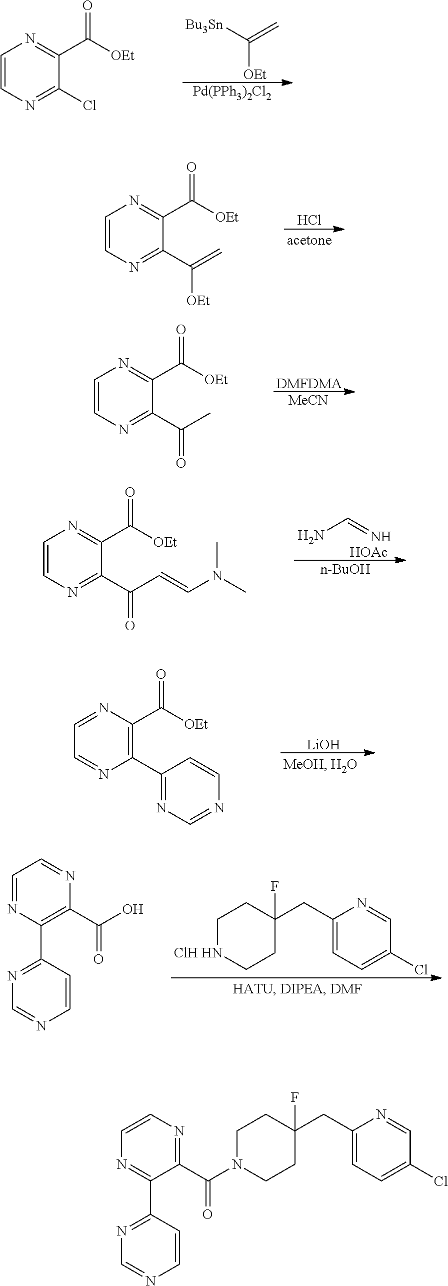

Step 1

To a solution of ethyl 3-chloropyrazine-2-carboxylate (5 g, 26.7 mmol) in toluene (50 mL) was added tributyl(1-ethoxyvinyl)stannane (11.5 g, 32.0 mmol) at 20° C. under N₂ and the mixture was stirred for 15 min. Then Pd(PPh₃)₂Cl₂ (933 mg, 1.33 mmol) was added and the mixture was stirred for another 5 min at 20° C. The reaction mixture was heated to 100° C. for 16 h and then poured into aq. KF (10 g in 200 mL water). The reaction mixture was diluted with water (100 mL) and extracted with EtOAc (2×100 mL). The combined organic extracts were dried over anhydrous Na₂SO₄, filtered, and concentrated under reduced pressure to afford ethyl 3-(1-ethoxyvinyl)pyrazine-2-carboxylate (6 g).

¹H NMR (400 MHz, CDCl₃) δ_{H} 8.60 (d, J=2.4 Hz, 1H), 8.52 (d, J=2.4 Hz, 1H), 5.20 (d, J=2.4 Hz, 1H), 4.52 (d, J=2.4 Hz, 1H), 4.43 (q, J=7.2 Hz, 2H), 3.94 (q, J=6.8 Hz, 2H), 1.41 (t, J=7.2 Hz, 3H), 1.36 (t, J=7.2 Hz, 3H).

Step 2

To a solution of ethyl 3-(1-ethoxyvinyl)pyrazine-2-carboxylate (6 g, 26.9 mmol) in acetone (150 mL) was added HCl (80.5 mL, 161 mmol, 2.0 M in water) at 20° C. and the mixture was stirred at 20° C. for 2 h. The volatiles were removed under reduced pressure and the mixture was poured into NaHCO₃ (200 mL, aq.). The reaction mixture was extracted with DCM (3×100 mL). The combined organic layers were washed with brine (300 mL), dried over Na₂SO₄, filtered, concentrated, and purified by column (0-30% EtOAc in PE) to give ethyl 3-acetylpyrazine-2-carboxylate (4.5 g).

¹H NMR (400 MHz, CDCl₃) δ_{H} 8.74 (d, J=2.4 Hz, 1H), 8.71 (d, J=2.4 Hz, 1H), 4.49 (q, J=7.2 Hz, 2H), 2.72 (s, 3H), 1.42 (t, J=7.2 Hz, 3H).

Step 3

To a solution of ethyl 3-acetylpyrazine-2-carboxylate (4.5 g, 23.1 mmol) in MeCN (50 mL) was added DMF-DMA (8.25 g, 69.3 mmol) at 20° C. and the mixture was stirred at 80° C. for 3 h. The reaction mixture was concentrated to afford ethyl (E)-3-(3-(dimethylamino)acryloyl)pyrazine-2-carboxylate (6 g).

¹H NMR (400 MHz, CDCl₃) δ_{H} 8.62 (s, 2H), 7.96-7.79 (m, 1H), 6.14 (m, 1H), 4.49 (q, J=7.2 Hz, 2H), 3.17 (s, 3H), 2.98 (s, 3H), 1.42 (t, J=7.2 Hz, 3H).

Step 4

To a mixture of ethyl (E)-3-(3-(dimethylamino)acryloyl)pyrazine-2-carboxylate (6 g, 24.0 mmol), acetic acid, and methanimidamide (31.2 g, 300 mmol) in n-BuOH (100 mL) was added DIPEA (58.6 mL, 336 mmol, 0.74 g/mL) at 25° C. and the mixture was stirred at 120° C. for 16 h. The mixture was poured into water (100 mL) and saturated NaHCO₃ (200 mL). The aqueous phase was extracted with EtOAc (3×100 mL) and the combined organic extracts were washed with brine (2×200 mL), dried over anhydrous Na₂SO₄, filtered and concentrated. The residue was purified by flash column (0-20% EtOAc in PE) to give ethyl 3-(pyrimidin-4-yl)pyrazine-2-carboxylate (2 g).

¹H NMR (400 MHz, CDCl₃) δ_{H} 9.25 (d, J=1.6 Hz, 1H), 8.95 (d, J=5.2 Hz, 1H), 8.77 (d, J=2.4 Hz, 1H), 8.72 (d, J=2.4 Hz, 1H), 8.21 (dd, J=1.6, 5.2 Hz, 1H), 4.47 (q, J=7.2 Hz, 2H), 1.36 (t, J=7.2 Hz, 3H).

Step 5

To a solution of ethyl 3-(pyrimidin-4-yl)pyrazine-2-carboxylate (2 g, 8.68 mmol) in MeOH (20 mL) and H₂O (2 mL) was added LiOH H₂O (436 mg, 10.4 mmol) at 25° C. and the mixture was stirred at 60° C. for 2 h. The reaction mixture was concentrated to give 3-(pyrimidin-4-yl)pyrazine-2-carboxylic acid (1.8 g).

¹H NMR (400 MHz, MeOD) δ_{H} 9.22 (d, J=1.2 Hz, 1H), 8.90 (d, J=5.2 Hz, 1H), 8.69 (d, J=2.4 Hz, 1H), 8.63 (d, J=2.4 Hz, 1H), 8.17 (dd, J=1.2, 5.2 Hz, 1H).

Step 6

To a solution of 3-(pyrimidin-4-yl)pyrazine-2-carboxylic acid (100 mg, 0.494 mmol) and HATU (281 mg, 0.740 mmol) in DMF (5 mL) was added DIPEA (0.428 mL, 2.46 mmol) at 20° C. 5-chloro-2-[(4-fluoropiperidin-4-yl)methyl]pyridine hydrochloride (130 mg, 0.494 mmol) in DMF (5 mL) was added slowly into the mixture. The mixture was stirred at 20° C. for 16 h. The reaction mixture poured into H₂O (10 mL) and stirred for 20 min. The aqueous phase was extracted with DCM (3×10 mL). The combined organic phase was washed with saturated brine (2×20 mL), dried over anhydrous Na₂SO₄, filtered, and concentrated. Purification by flash column (0-5% MeOH in DCM) and further purification by Prep-HPLC (Column: Phenomenex C18 80*40 mm*3 um; Condition: water (NH₃H₂O)-ACN; Begin B: 26; End B: 56; Gradient Time(min): 8; 100% B Hold Time(min): 2.2; Flow Rate (ml/min): 30; Injections: 5) provided (4-((5-chloropyridin-2-yl)methyl)-4-fluoropiperidin-1-yl)(3-(pyrimidin-4-yl)pyrazin-2-yl)methanone (56 mg, 27%).

¹H NMR (400 MHz, CDCl₃) δ_{H} 9.09 (d, J=1.2 Hz, 1H), 8.91 (d, J=5.2 Hz, 1H), 8.70 (d, J=2.4 Hz, 1H), 8.65 (d, J=2.4 Hz, 1H), 8.50 (d, J=2.4 Hz, 1H), 8.20 (dd, J=1.6, 5.2 Hz, 1H), 7.63 (dd, J=2.4, 8.4 Hz, 1H), 7.24 (d, J=8.4 Hz, 1H), 4.59 (d, J=13.6 Hz, 1H), 3.53-3.34 (m, 2H), 3.31-3.21 (m, 1H), 3.20 (d, J=3.2 Hz, 1H), 3.14 (d, J=4.8 Hz, 1H), 2.12-1.87 (m, 3H), 1.77-1.67 (m, 1H). ¹⁹F NMR (376.5 MHz, CDCl₃). δ_{F} −160.054. LC-ELSD/MS purity 99%, MS ESI calcd. For C₂₀H₁₈ClFN₆O [M+Na]⁺ 435.2, found 435.2.

### Example 94. Synthesis of (4-fluoro-4-(imidazo[1,2-a]pyridin-7-ylmethyl)piperidin-1-yl)(2-(pyrimidin-4-yl)pyridin-3-yl)methanone (Cmpd 90)

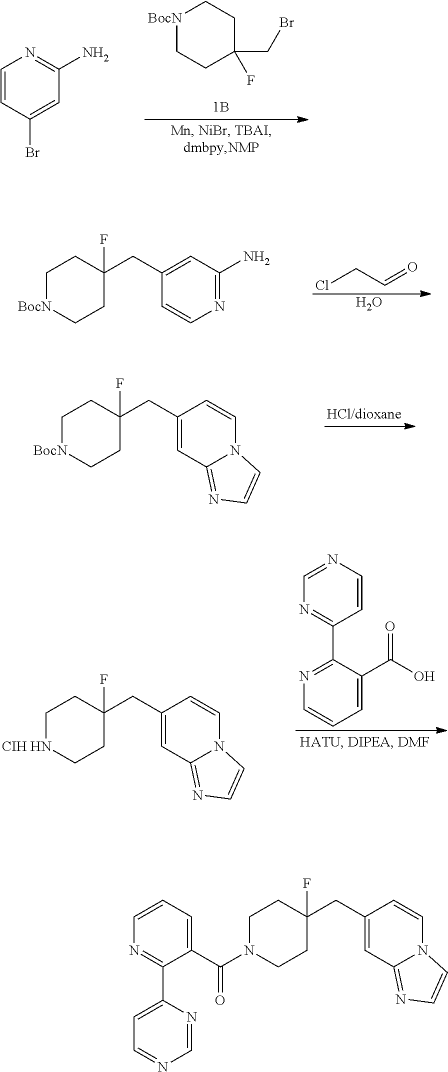

Step 1

To a mixture of 4-bromopyridin-2-amine (1 g, 5.77 mmol) and tert-butyl 4-(bromomethyl)-4-fluoropiperidine-1-carboxylate (2.04 g, 6.92 mmol) in NMP (15 mL) was added nickel dibromide (126 mg, 577 μmol), manganese (1.26 g, 23.0 mmol), tetrabutylazanium iodide (424 mg, 1.15 mmol), and 4,4′-dimethyl-2,2′-bipyridine (106 mg, 577 μmol). The mixture was stirred at 80° C. under N₂ for 48 hours. The mixture was cooled and concentrated under reduced pressure at 40° C. The residue was poured into saturated ammonium chloride (30 mL) and stirred for 20 min. The aqueous phase was extracted with EtOAc (3×20 mL). The combined organic phase was washed with brine (2×50 mL), dried over anhydrous Na₂SO₄, filtered, and concentrated. Purification by flash column (0˜25% of DCM in MeOH) provided tert-butyl 4-((2-aminopyridin-4-yl)methyl)-4-fluoropiperidine-1-carboxylate (1.15 g). LCMS: purity 97.9%, MS ESI calcd. for C₁₆H₂₄FN₂O₂[M+H]⁺ 310.2, found 310.2.

Step 2

A solution of tert-butyl 4-((2-aminopyridin-4-yl)methyl)-4-fluoropiperidine-1-carboxylate (1.15 g, 3.71 mmol) and 2-chloroacetaldehyde (1.81 g, 40% in H₂O, 9.27 mmol) in H₂O (10 mL) at 20° C. under N₂ was stirred at 20° C. for 10 min then heated at 80° C. for 12 h. The mixture was cooled, concentrated, and adjusted to pH 10 by addition of aqueous saturated Na₂CO₃. The aqueous phase was extracted with DCM (3×20 mL). The combined organic phase was washed with brine (2×20 mL), dried over anhydrous Na₂SO₄, filtered, and concentrated to give tert-butyl 4-fluoro-4-(imidazo[1,2-a]pyridin-7-ylmethyl)piperidine-1-carboxylate (1.1 g).

¹H NMR (400 MHz, CDCl₃) δ_{H} 8.03-7.93 (m, 1H), 7.58-7.44 (m, 2H), 7.42-7.29 (m, 1H), 6.77-6.59 (m, 1H), 3.22-2.92 (m, 3H), 2.91-2.87 (m, 1H), 2.86-2.75 (m, 2H), 2.42-1.76 (m, 13H). ¹⁹F NMR (376.5 MHz, CDCl₃) δ_{F} −161.864.

Step 3

To a mixture of tert-butyl 4-fluoro-4-(imidazo[1,2-a]pyridin-7-ylmethyl)piperidine-1-carboxylate (1.1 g, 3.29 mmol) in dioxane (10 mL) was added HCl/dioxane (8.22 mL, 4M in dioxane, 32.9 mmol) and the mixture was stirred at 25° C. for 2 h. The mixture was cooled and concentrated to give 7-((4-fluoropiperidin-4-yl)methyl)imidazo[1,2-a]pyridine hydrochloride (1 g), which was carried directly to the next step.

Step 4

To a solution of 2-(pyrimidin-4-yl)pyridine-3-carboxylic acid (446 mg, 2.22 mmol), HOBT (374 mg, 2.77 mmol) and (3-{[(ethylimino)methylidene]amino}propyl)dimethylamine hydrochloride (531 mg, 2.77 mmol) in DMF (10 mL) was added DIPEA (1.6 mL, 9.25 mmol) at 20° C. 7-((4-fluoropiperidin-4-yl)methyl)imidazo[1,2-a]pyridine hydrochloride (500 mg, 1.85 mmol) in DMF (10 mL) was added slowly. The mixture was stirred at 20° C. for 1-2 h. The reaction mixture poured into H₂O (20 mL) and stirred for 20 min. The aqueous phase was extracted with EtOAc (3×10 mL). The combined organic phase was washed with brine (2×10 mL), dried over anhydrous Na₂SO₄, filtered, and concentrated. Purification by Prep-HPLC (Column: Phenomenex C18 80*40 mm*3 um; Condition: water (NH₃H₂O)-ACN; Begin B: 20; End B: 50; Gradient Time (min): 8; 100% B Hold Time (min): 2; FlowRate (ml/min): 30; Injections: 8) provided (4-fluoro-4-(imidazo[1,2-a]pyridin-7-ylmethyl)piperidin-1-yl)(2-(pyrimidin-4-yl)pyridin-3-yl)methanone. Further purification by SFC (Column: DAICEL CHIRALPAK AS (250 mm*30 mm, 10 um); Condition: 0.1% NH₃H₂O ETOH; Begin B: 35; End B: 35; FlowRate(ml/min): 80; Injections: 30) provided (4-fluoro-4-(imidazo[1,2-a]pyridin-7-ylmethyl)piperidin-1-yl)(2-(pyrimidin-4-yl)pyridin-3-yl)methanone (5.6 mg, 18%).

¹H NMR (400 MHz, CDCl₃) δ_{H} 9.22-8.87 (m, 1H), 8.85 (d, J=5.2 Hz, 1H), 8.74 (dd, J=1.6, 4.6 Hz, 1H), 8.24 (dd, J=4.9, 14.1 Hz, 1H), 8.07 (d, J=6.8 Hz, 1H), 7.75-7.53 (m, 3H), 7.50-7.36 (m, 2H), 6.78-6.61 (m, 1H), 4.79-4.48 (m, 1H), 3.40 (s, 1H), 3.28-2.86 (m, 4H), 2.15-1.75 (m, 3H), 1.25 (s, 1H). ¹⁹F NMR (376.5 MHz, CDCl₃) δ_{F} −161.197. LCMS purity 95.54%, MS ESI calcd. for C₂₃H₂₁FN₆O [M+H]⁺ 417.2, found 417.2.

### Example 95. Synthesis of 3-(4-((5-chloropyridin-2-yl)methyl)-4-fluoropiperidine-1-carbonyl)-[2,4′-bipyridine]-6-carbonitrile (Cmpd 91)

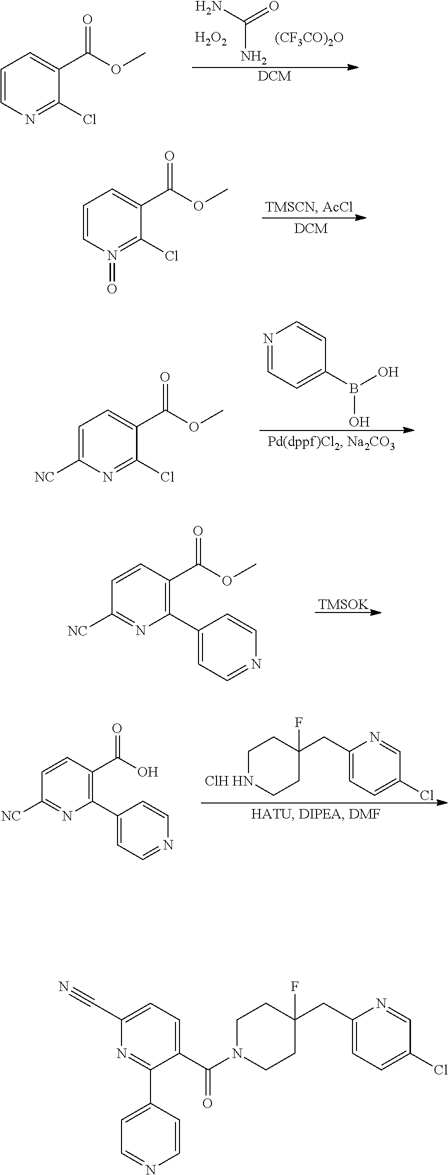

Step 1

To a solution of methyl 2-chloronicotinate (5 g, 29.1 mmol) and urea hydrogen peroxide (5.47 g, 58.2 mmol) in DCM (50 mL) was added TFAA (12.2 g, 58.2 mmol) dropwise at 0° C. and the resulting mixture was warmed to 25° C. with stirring under N₂ for 16 h. The mixture was added to cooled sodium carbonate (50 mL) and Na₂S2O₃ (50 mL) and adjusted to a pH of 8-9. The mixture was extracted with DCM (100 mL×2). The combined organic layers were dried over sodium sulfate, filtered, and concentrated to give 2-chloro-3-(methoxycarbonyl)pyridine 1-oxide (5 g).

¹H NMR (400 MHz, CDCl₃) δ_{H} 8.46 (d, J=6.4 Hz, 1H), 7.67 (d, J=8.0 Hz, 1H), 7.29-7.24 (m, 1H), 3.98 (d, J=1.2 Hz, 3H).

Step 2

To a solution of 2-chloro-3-(methoxycarbonyl)pyridine 1-oxide (5 g, 26.6 mmol) and TMSCN (3.95 g, 39.9 mmol) in DCM (50 mL) was added acetyl chloride (4.16 g, 53 mmol) dropwise at 15° C. and the mixture was stirred at 25° C. under N₂ for 16 h. Then TMSCN (3.96 g, 39.9 mmol) and acetyl chloride (4.16 g, 53 mmol) were added into the mixture again and the mixture was stirred at 25° C. for another 4 h. The mixture was poured into saturated sodium carbonate (100 mL) and extracted with DCM (100 mL×2). The combined organic layers were dried over sodium sulfate, filtered, and concentrated. The residue was purified by flash column (0-35% EtoAc in PE) to give methyl 2-chloro-6-cyanonicotinate (5 g).

¹H NMR (400 MHz, CDCl₃) δ_{H} 8.28 (d, J=7.6 Hz, 1H), 7.72 (d, J=7.6 Hz, 1H), 4.00 (s, 3H).

Step 3

A mixture of Pd(dppf)Cl₂ (185 mg, 0.254 mmol), Na₂CO₃ (1.07 g, 10.1 mmol), methyl 2-chloro-6-cyanonicotinate (1 g, 5.08 mmol) and (pyridin-4-yl)boronic acid (748 mg, 6.09 mmol) in dioxane (10 mL) and water (2 mL) was stirred at 90° C. for 16 h under N₂. After cooling to 20° C., the residue was poured into water (50 mL) and the mixture was extracted with EtOAc (30 mL×2). The combined organic phase was washed with brine (50 mL), dried over Na₂SO₄, filtered, and concentrated. Purification by flash column (0-60% EtOAc in PE) provided methyl 6-cyano-[2,4′-bipyridine]-3-carboxylate (550 mg).

¹H NMR (400 MHz, CDCl₃) δ_{H} 8.77-8.69 (m, 2H), 8.31 (d, J=8.0 Hz, 1H), 7.82 (d, J=8.0 Hz, 1H), 7.48-7.41 (m, 2H), 3.77 (s, 3H).

Step 4

To a solution of methyl 6-cyano-[2,4′-bipyridine]-3-carboxylate (200 mg, 0.836 mmol) in dioxane (4 mL) was added TMSOK (128 mg, 1.00 mmol) at 25° C. and the mixture was stirred at 25° C. for 2 h. The reaction mixture was poured into citric acid monohydrate (10 mL) and adjusted to a pH of 5-6. The mixture was extracted with DCM (10 mL×2). The combined organic layers were dried over sodium sulfate, filtered, and concentrated to give 6-cyano-[2,4′-bipyridine]-3-carboxylic acid (30 mg).

¹H NMR (400 MHz, MeOD) δ_{H} 8.73-8.68 (m, 2H), 8.43 (d, J=7.6 Hz, 1H), 8.03 (d, J=8.0 Hz, 1H), 7.80-7.76 (m, 2H).

Step 5

To a solution of 6-cyano-[2,4′-bipyridine]-3-carboxylic acid (30 mg, 0.133 mmol) and HATU (75.6 mg, 0.199 mmol) in DMF (2 mL) was added DIPEA (0.116 mL, 0.665 mmol, 0.74 g/mL) at 20° C. and the mixture was stirred for 15 min. 5-chloro-2-[(4-fluoropiperidin-4-yl)methyl]pyridine hydrochloride (35.2 mg, 0.133 mmol) in DMF (2 mL) was added slowly into the mixture. The mixture was stirred at 20° C. for 16 h. The reaction mixture was poured into H₂O (10 mL) and stirred for 10 min. The aqueous phase was extracted with DCM (3×10 mL). The combined organic phase was washed with saturated brine (2×20 mL), dried over anhydrous Na₂SO₄, filtered, and concentrated. Purification by flash column (0-5% MeOH in DCM) and further purification by Prep-HPLC (Column: Phenomenex C18 80*40 mm*3 um; Condition: water (NH₃H₂O)-ACN; Begin B: 32; End B: 62; Gradient Time(min): 8; 100% B Hold Time(min): 2; Flow Rate (ml/min): 30; Injections: 4) provided 3-(4-((5-chloropyridin-2-yl)methyl)-4-fluoropiperidine-1-carbonyl)-[2,4′-bipyridine]-6-carbonitrile (25 mg, 43%).

¹H NMR (400 MHz, CDCl₃) δ_{H} 8.74 (m, 2H), 8.46 (m, 1H), 7.97-7.86 (m, 1H), 7.80 (d, J=8.0 Hz, 1H), 7.72 (d, J=6.0 Hz, 1H), 7.65-7.56 (m, 2H), 7.19-7.00 (m, 1H), 4.54 (m, 1H), 3.25-2.96 (m, 2H), 2.92-2.57 (m, 3H), 1.93-1.74 (m, 2H), 1.55-1.27 (m, 1H), 0.35-0.13 (m, 1H). ¹⁹F NMR (376.5 MHz, CDCl₃). δ_{F} −160.529, 161.917. LCMS purity 99%, MS ESI calcd. For C₂₃H₁₉ClFN₅O [M+H]⁺ 436.1, found 436.1.

### Example 96. Synthesis of (4-((5-chloropyridin-2-yl)methyl)-4-fluoropiperidin-1-yl)(3-(pyrimidin-4-yl)pyridazin-4-yl)methanone (Cmpd 92)

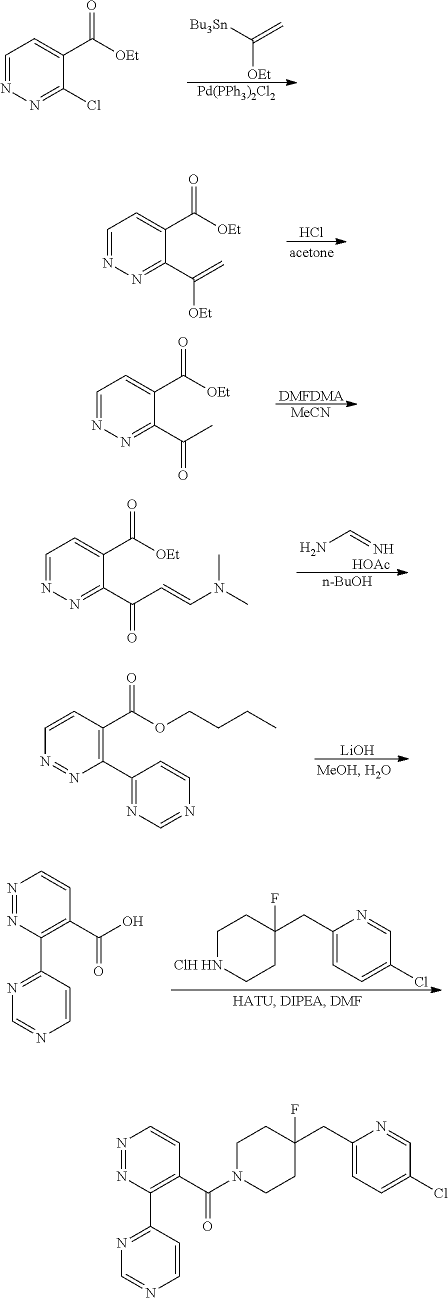

Step 1

To a solution of ethyl 3-chloropyridazine-4-carboxylate (3 g, 16.0 mmol) in toluene (30 mL) was added tributyl(1-ethoxyvinyl)stannane (6.93 g, 19.2 mmol) at 20° C. under N₂ and the mixture was for 15 min. Then Pd(PPh₃)₂Cl₂ (561 mg, 0.8 mmol) was added and the mixture was stirred for another 5 min at 20° C. The reaction mixture was heated to 100° C. for 16 h then poured into aq. KF (10 g in 100 mL water) and extracted with EtOAc (2×100 mL). The combined organic extracts were dried over anhydrous Na₂SO₄, filtered, and concentrated to afford ethyl 3-(1-ethoxyvinyl)pyridazine-4-carboxylate (4 g), which was used directly in the next step without further purification.

¹H NMR (400 MHz, CDCl₃) δ_{H} 9.25 (d, J=5.2 Hz, 1H), 7.56 (d, J=5.2 Hz, 1H), 5.30 (d, J=2.4 Hz, 1H), 4.58 (d, J=2.4 Hz, 1H), 4.38 (q, J=7.2 Hz, 2H), 3.94 (q, J=7.2 Hz, 2H), 1.40-1.32 (m, 6H).

Step 2

To a solution of ethyl 3-(1-ethoxyvinyl)pyridazine-4-carboxylate (4 g, 17.9 mmol) in acetone (50 mL) was added HCl (42.8 mL, 107 mmol, 2.5 M in water) at 20° C. and the mixture was stirred for 2 h. The volatiles were removed under reduced pressure and poured into NaHCO₃ (200 mL, aq.) The reaction mixture was extracted with DCM (2×100 mL). The combined organic layers were washed with brine (200 mL) and dried over Na₂SO₄, filtered, concentrated, and purified by flash column (0-30% EtOAc in PE) to give ethyl 3-acetylpyridazine-4-carboxylate (3 g).

¹H NMR (400 MHz, CDCl₃) δ_{H} 9.42 (d, J=5.2 Hz, 1H), 7.69 (d, J=5.2 Hz, 1H), 4.43 (q, J=7.2 Hz, 2H), 2.88 (s, 3H), 1.39 (t, J=7.2 Hz, 3H).

Step 3

To a solution of ethyl 3-acetylpyridazine-4-carboxylate (3 g, 15.4 mmol) in MeCN (30 mL) was added DMF-DMA (5.50 g, 46.2 mmol) at 20° C. and the mixture was stirred at 80° C. for 3 h. The reaction mixture was concentrated to afford ethyl (E)-3-(3-(dimethylamino)acryloyl)pyridazine-4-carboxylate (3.5 g).

Step 4

To a mixture of ethyl (E)-3-(3-(dimethylamino)acryloyl)pyridazine-4-carboxylate (3.5 g, 14.0 mmol), acetic acid, and methanimidamide (18.2 g, 175 mmol) in n-BuOH (40 mL) was added DIPEA (34.1 mL, 196 mmol, 0.74 g/mL) at 25° C., and the mixture was stirred at 120° C. for 16 h. The residue was poured into water (100 mL) and saturated NaHCO₃ (200 mL). The aqueous phase was extracted with DCM (3×100 mL) and the combined organic extracts were washed with brine (2×200 mL), dried over anhydrous Na₂SO₄, filtered, and concentrated. The residue was purified by flash column (0˜30% EtOAc in PE) to give butyl 3-(pyrimidin-4-yl)pyridazine-4-carboxylate (1.2 g).

¹H NMR (400 MHz, CDCl₃) δ_{H} 9.41 (d, J=5.2 Hz, 1H), 9.26 (d, J=1.2 Hz, 1H), 8.99 (d, J=5.2 Hz, 1H), 8.39 (m, 1H), 7.73 (d, J=5.2 Hz, 1H), 4.31 (t, J=6.8 Hz, 2H), 1.61-1.54 (m, 2H), 1.28-1.24 (m, 2H), 0.89 (t, J=7.6 Hz, 3H).

Step 5

To a solution of butyl 3-(pyrimidin-4-yl)pyridazine-4-carboxylate (1.2 g, 4.64 mmol) in MeOH (10 mL) and H₂O (1 mL) was added LiOH H₂O (233 mg, 5.56 mmol) at 25° C. then the mixture was stirred at 60° C. for 2 h. The reaction mixture was concentrated to give 3-(pyrimidin-4-yl)pyridazine-4-carboxylic acid (1.1 g).

¹H NMR (400 MHz, MeOD) δ_{H} 9.28 (d, J=5.2 Hz, 1H), 9.25 (s, 1H), 8.95 (d, J=5.2 Hz, 1H), 8.18 (dd, J=1.2, 5.2 Hz, 1H), 7.80 (d, J=5.2 Hz, 1H).

Step 6

To a solution of 3-(pyrimidin-4-yl)pyridazine-4-carboxylic acid (100 mg, 0.494 mmol) and HATU (281 mg, 0.740 mmol) in DMF (5 mL) was added DIPEA (0.428 mL, 2.46 mmol, 0.74 g/mL) at 20° C. and stirred for 15 min. 5-chloro-2-[(4-fluoropiperidin-4-yl)methyl]pyridine hydrochloride (130 mg, 0.494 mmol) in DMF (5 mL) was added slowly into the mixture. The mixture was stirred at 20° C. for 16 h. The reaction mixture was poured into H₂O (10 mL) and stirred for 10 min. The aqueous phase was extracted with DCM (3×10 mL). The combined organic phase was washed with saturated brine (2×20 mL), dried over anhydrous Na₂SO₄, filtered, and concentrated. Purification by flash column (0-5% MeOH in DCM) and further purification by Prep-HPLC (Column: Phenomenex C18 80*40 mm*3 um; Condition: water (NH₃H₂O)-ACN; Begin B: 24; End B: 54; Gradient Time(min): 8; 100% B Hold Time(min): 2; FlowRate(ml/min): 30; Injections: 6) provided (4-((5-chloropyridin-2-yl)methyl)-4-fluoropiperidin-1-yl)(3-(pyrimidin-4-yl)pyridazin-4-yl)methanone (76 mg, 37%).

¹H NMR (400 MHz, CDCl₃) δ_{H} 9.32 (d, J=5.2 Hz, 1H), 9.27-8.91 (m, 2H), 8.59-8.40 (m, 2H), 7.63 (d, J=7.6 Hz, 1H), 7.43 (dd, J=4.4, 9.2 Hz, 1H), 7.22 (d, J=8.0 Hz, 1H), 4.72-4.44 (m, 1H), 3.52-3.21 (m, 2H), 3.21-3.07 (m, 3H), 2.07-1.85 (m, 2H), 1.78-1.64 (m, 2H). ¹⁹F NMR (376.5 MHz, CDCl₃). δ_{F} −159.962, 160.266. LCMS purity 99%, MS ESI calcd. For C₂₀H₁₈ClFN₆O [M+H]⁺ 413.2, found 413.2.

### Example 97. Synthesis of 2-((4-fluoro-1-(2-(pyrimidin-4-yl)nicotinoyl)piperidin-4-yl)methyl)isonicotinonitrile (Cmpd 93)

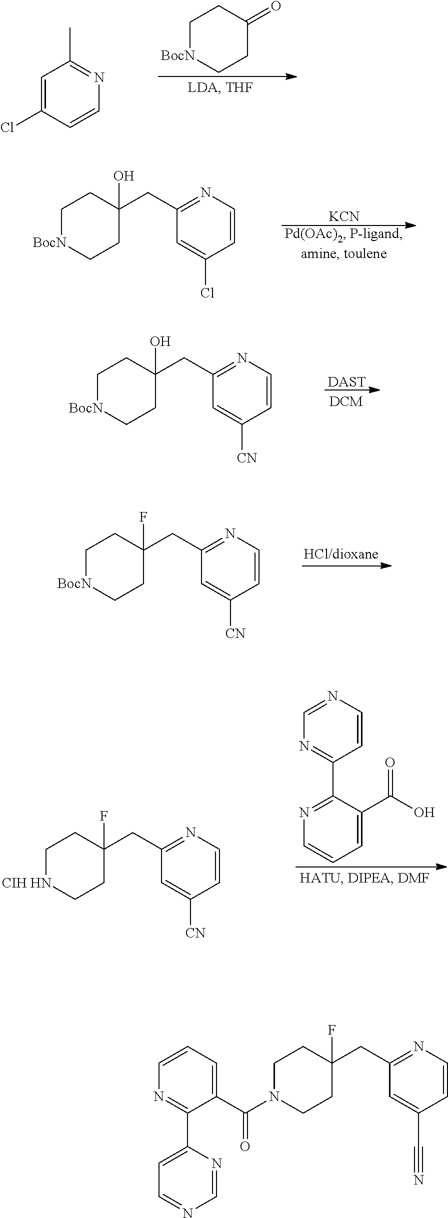

Step 1

To a LDA solution was added 4-chloro-2-methylpyridine (5 g, 39.1 mmol) and the mixture was stirred at −70° C. for 1 h. Then tert-butyl 4-oxopiperidine-1-carboxylate (7.79 g, 39.1 mmol) in THE (80 mL) was added and the mixture was stirred at −70° C. for 3 h. The mixture was poured into saturated ammonium chloride solution (100 mL) and extracted with EtOAc (50 mL×3). The combined organic phase was washed with brine (2×50 mL), dried over Na₂SO₄, filtered, and concentrated. The residue was purified by flash column (0-35% of EtOAc in PE) to give tert-butyl 4-((4-chloropyridin-2-yl)methyl)-4-hydroxypiperidine-1-carboxylate (13 g).

¹H NMR (400 MHz, CDCl₃) δ_{H} 8.42 (d, J=5.5 Hz, 1H), 7.20 (d, J=1.5 Hz, 1H), 3.97-3.61 (m, 2H), 3.21 (s, 2H), 2.93 (s, 2H), 1.51 (s, 2H), 1.50-1.47 (m, 2H), 1.45 (s, 9H)

Step 2

A mixture of tert-butyl 4-((4-chloropyridin-2-yl)methyl)-4-hydroxypiperidine-1-carboxylate (5.0 g, 15.2 mmol), Pd(OAc)₂ (170 mg, 760 μmoll), 1,4-bis(diphenylphosphino)butane (648 mg, 1.52 mmol), tetramethylethylenediamine (883 mg, 7.60 mmol), and potassium cyanide (989 mg, 15.2 mmol) in toluene (30 ml) at 20° C. under N₂ was heated to 115° C. After stirring at 115° C. for 12 h, the reaction was cooled to room temperature and extracted with EtOAc (2×50 mL). The combined organic phase was washed with water (2×100 mL), dried over Na₂SO₄, filtered, and concentrated. Purification by silica gel chromatography (10-30% of EtOAc in PE) provided tert-butyl 4-((4-cyanopyridin-2-yl)methyl)-4-hydroxypiperidine-1-carboxylate (2.02 g, 42%).

¹H NMR (400 MHz, CDCl₃) δ_{H} 8.70 (d, J=5.0 Hz, 1H), 7.43 (d, J=5.0 Hz, 1H), 7.38 (s, 1H), 3.94-3.08 (m, 4H), 2.98 (s, 2H), 1.50 (dd, J=3.9, 6.9 Hz, 4H), 1.45 (s, 9H)

Step 3

To a mixture of tert-butyl 4-((4-cyanopyridin-2-yl)methyl)-4-hydroxypiperidine-1-carboxylate (2 g, 6.30 mmol) in DCM (40 mL) was added DAST (2.03 g, 12.6 mmol) at 0° C. The mixture was stirred at 0° C. for 10 min. The reaction mixture was added to NaHCO₃ (20 mL) slowly. The organic layers were washed with NaHCO₃ (50 mL) and brine (50 mL), dried over anhydrous Na₂SO₄, filtered, and concentrated. The residue was purified by flash column (0˜40% of EtOAc in PE) to give tert-butyl 4-((4-cyanopyridin-2-yl)methyl)-4-fluoropiperidine-1-carboxylate (2 g).

Step 4

To a mixture of tert-butyl 4-((4-cyanopyridin-2-yl)methyl)-4-fluoropiperidine-1-carboxylate (2 g, 6.26 mmol) in dioxane (30 mL) was added HCl/dioxane (20 mL, 4M in dioxane, 1.72 mmol) and the mixture was stirred at 25° C. for 0.5 h. The mixture was concentrated to give 2-((4-fluoropiperidin-4-yl)methyl)isonicotinonitrile hydrochloride (2 g), which was used directly in the next step.

Step 5

To a solution of [2-(pyrimidin-4-yl) pyridine-3-carboxylic acid (549 mg, 2.73 mmol) and HATU (1.55 g, 4.09 mmol) in DMF (20 mL) was added DIPEA (2.36 mL, 13.6 mmol). 2-((4-fluoropiperidin-4-yl)methyl)isonicotinonitrile hydrochloride (600 mg, 2.73 mmol) in DMF (10 mL) was added slowly. The mixture was stirred at 20° C. for 1-2 h. The reaction mixture was poured into H₂O (20 mL) and stirred for 20 min. The aqueous phase was extracted with EtOAc (3×10 mL). The combined organic phase was washed with saturated brine (2×20 mL), dried over anhydrous Na₂SO₄, filtered, and concentrated. The residue was purified by flash column (0˜10% of MeOH in DCM), further purified by SFC (Column: DAICEL CHIRALCEL OD-H (250 mm*30 mm, 5 um); Condition: 0.1% NH₃H₂O ETOH; Begin B: 30%; End B: 30%; FlowRate (ml/min): 80; Injections: 180), and purified again by SFC (Column: DAICEL CHIRALCEL OJ (250 mm*30 mm, 10 um); Condition: 0.1% NH₃H₂O ETOH; Begin B: 20%; End B: 20%; FlowRate (ml/min): 60; Injections: 35) to afford 2-((4-fluoro-1-(2-(pyrimidin-4-yl)nicotinoyl)piperidin-4-yl)methyl)iso nicotinonitrile (55 mg, 18%).

¹H NMR (400 MHz, CDCl₃) δ_{H} 9.23-8.96 (m, 1H), 8.91-8.84 (m, 1H), 8.79-8.68 (m, 2H), 8.29-8.20 (m, 1H), 7.72-7.64 (m, 1H), 7.51 (br d, J=8.0 Hz, 1H), 7.45 (dd, J=4.6, 7.7 Hz, 2H), 4.72-4.50 (m, 1H), 3.48-3.36 (m, 1H), 3.30-3.13 (m, 4H), 2.13-1.64 (m, 4H). ¹⁹F NMR (376.5 MHz, CDCl₃) δ_{F} −160.29. LCMS purity 97%; MS ESI calcd. for C₂₂H₁₉FN₆O [M+H]⁺ 403.1, found 403.1.

### Example 98. Synthesis of 2-((1-([2,4′-bipyridine]-3-carbonyl)-4-fluoropiperidin-4-yl) methyl)isonicotinonitrile (Cmpd 94)

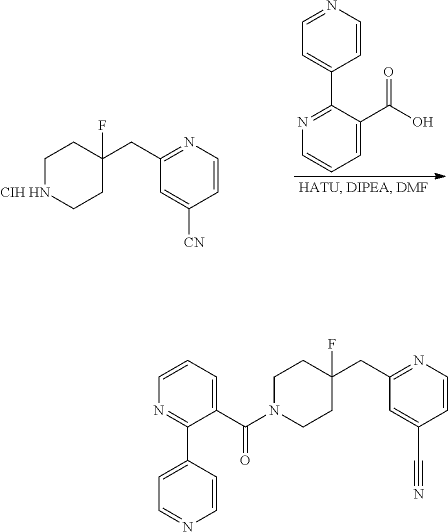

To a solution of [2,4′-bipyridine]-3-carboxylic acid (546 mg, 2.73 mmol) and HATU (1.55 g, 4.09 mmol) in DMF (20 mL) was added DIPEA (2.36 mL, 13.6 mmol) at 20° C. 2-((4-fluoropiperidin-4-yl)methyl)isonicotinonitrile hydrochloride (600 mg, 0.91 mmol) in DMF (10 mL) was added slowly. The mixture was stirred at 20° C. for 1-2 h. The reaction mixture was poured into H₂O (20 mL) and stirred for 20 min. The aqueous phase was extracted with EtOAc (3×10 mL). The combined organic phase was washed with saturated brine (2×20 mL), dried over anhydrous Na₂SO₄, filtered, and concentrated. Purification by flash column (0-10% of MeOH in DCM) and further purification by SFC (Column: DAICEL CHIRALPAK AD (250 mm*30 mm, 10 um); Condition: 0.1% NH₃H₂O ETOH; Begin B: 35; End B: 35; FlowRate (ml/min): 80; Injections: 150) provided 2-((1-([2,4′-bipyridine]-3-carbonyl)-4-fluoropiperidin-4-yl)methyl)isonicotinonitrile (67 mg, 22%).

¹H NMR (400 MHz, CDCl₃) δ_{H} 8.78 (dd, J=1.8, 4.8 Hz, 1H), 8.74-8.66 (m, 3H), 7.81-7.70 (m, 2H), 7.60 (d, J=5.3 Hz, 1H), 7.43 (dd, J=4.8, 7.8 Hz, 2H), 7.33 (s, 1H), 4.67-4.48 (m, 1H), 3.27-2.73 (m, 5H), 1.92-1.66 (m, 2H), 1.54-1.23 (m, 2H). ¹⁹F NMR (376.5 MHz, CDCl₃) S_{F}-161.68. LCMS purity 96.1%; MS ESI calcd. for C₂₃H₂₀FN₅O [M+H]⁺ 402.2, found 402.2.

### Example 99. Synthesis of 6-((1-([2,4′-bipyridine]-3-carbonyl)-4-fluoropiperidin-4-yl)methyl)nicotinonitrile (Cmpd 97)

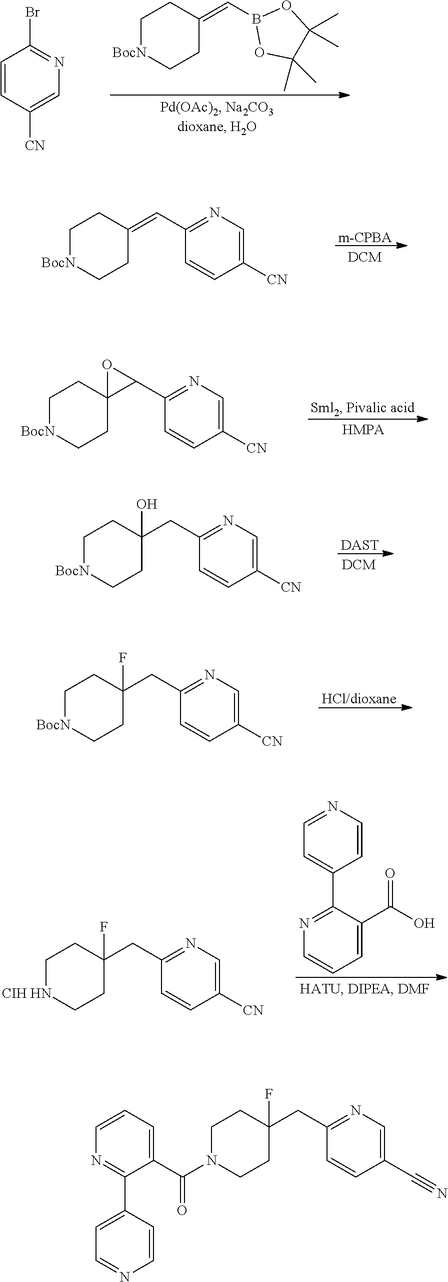

Step 1

A mixture of Pd(dppf)Cl₂ (199 mg, 0.273 mmol), Na₂CO₃ (1.15 g, 10.9 mmol), 6-bromonicotinonitrile (1 g, 5.46 mmol) and tert-butyl 4-[(4,4,5,5-tetramethyl-1,3,2-dioxaborolan-2-yl)methylidene]piperidine-1-carboxylate (1.76 g, 5.46 mmol) in dioxane (10 mL) and water (2 mL) was stirred at 90° C. for 16 h under N₂. The mixture was cooled to 20° C., filtered and concentrated. Purification by flash column (0-15% EtOAc in PE) provided tert-butyl 4-((5-cyanopyridin-2-yl)methylene)piperidine-1-carboxylate (1.5 g).

¹H NMR (400 MHz, CDCl₃) δ_{H} 8.81 (d, J=1.6 Hz, 1H), 7.86 (dd, J=2.4, 8.4 Hz, 1H), 7.21 (d, J=8.0 Hz, 1H), 6.36 (s, 1H), 3.55 (t, J=5.6 Hz, 2H), 3.48 (t, J=5.6 Hz, 2H), 2.97 (t, J=5.6 Hz, 2H), 2.40 (t, J=5.6 Hz, 2H), 1.48 (s, 9H).

Step 2

To a solution of tert-butyl 4-((5-cyanopyridin-2-yl)methylene)piperidine-1-carboxylate (1.5 g, 5.01 mmol) in DCM (30 mL) was added m-CPBA (5.07 g, 25.0 mmol, 85% purity) at 0° C. and the reaction mixture was stirred at 0° C. for 5 h. The reaction mixture was diluted with Na₂S₂O₃ (50 mL) and NaHCO₃ (50 mL), and extracted with DCM (50 mL×2). The combined organic phase was dried over Na₂SO₄, filtered, and concentrated. Purification by flash column (0-20% EtOAc in PE) provided tert-butyl 2-(5-cyanopyridin-2-yl)-1-oxa-6-azaspiro[2.5]octane-6-carboxylate (900 mg).

Step 3

To a solution of tert-butyl 2-(5-cyanopyridin-2-yl)-1-oxa-6-azaspiro[2.5]octane-6-carboxylate (900 mg, 2.85 mmol) in HMPA (28.5 mL) was added SmI₂ (71.2 mL, 0.1 M in THF, 7.12 mmol) at 20° C. A solution of pivalic acid (23.4 mL, 0.17 M in THF, 3.95 mmol) was added and the solution was stirred for 16 h at 20° C. The reaction was quenched with a solution of sodium potassium tartrate (100 mL), extracted with EtOAc (2×100 mL), and the combined organic layer was washed with brine (2×100 mL), dried over Na₂SO₄, filtered, and concentrated. Purification by flash column (0-20% EtOAc in PE) provided tert-butyl 4-((5-cyanopyridin-2-yl)methyl)-4-hydroxypiperidine-1-carboxylate (400 mg).

¹H NMR (400 MHz, CDCl₃) δ_{H} 8.79 (d, J=2.0 Hz, 1H), 7.90 (dd, J=2.0, 8.4 Hz, 1H), 7.29 (d, J=8.4 Hz, 1H), 4.86 (s, 1H), 3.89-3.73 (m, 2H), 3.28-3.11 (m, 2H), 2.99 (s, 2H), 1.52-1.47 (m, 4H), 1.43 (s, 9H).

Step 4

To a mixture of tert-butyl 4-((5-cyanopyridin-2-yl)methyl)-4-hydroxypiperidine-1-carboxylate (400 mg, 1.26 mmol) in DCM (10 mL) was added DAST (406 mg, 2.52 mmol) at 0° C. The mixture was stirred at 0° C. for 30 min. The mixture was poured into water (10 mL) and NaHCO₃ (10 mL) and stirred for 10 min. The aqueous phase was extracted with DCM (2×20 mL). The combined organic phase was washed with saturated brine (2×20 mL), dried over anhydrous Na₂SO₄, filtered, and concentrated. Purification by flash column (0-40% EtOAc in PE) and further purification by SFC (Column: DAICEL CHIRALCEL OD (250 mm*30 mm, 10 um); Condition: 0.1% NH₃H₂O ETOH; Begin B: 15; End B: 15; Flow Rate (ml/min): 60; Injections 70) provided tert-butyl 4-((5-cyanopyridin-2-yl)methyl)-4-fluoropiperidine-1-carboxylate (40 mg).

¹H NMR (400 MHz, CDCl₃) δ_{H} 8.82 (d, J=1.6 Hz, 1H), 7.90 (dd, J=2.4, 8.0 Hz, 1H), 7.41 (d, J=8.0 Hz, 1H), 4.01-3.81 (m, 2H), 3.22-3.14 (m, 2H), 3.10-2.99 (m, 2H), 1.78-1.60 (m, 4H), 1.45 (s, 9H). ¹⁹F NMR (376.5 MHz, CDCl₃) δ_{F}−160.25.

Step 5

To a solution of tert-butyl 4-((5-cyanopyridin-2-yl)methyl)-4-fluoropiperidine-1-carboxylate (40 mg, 0.125 mmol) in 1,4-dioxane (5 mL) was added hydrogen chloride (5 mL, 20.0 mmol, 4 M in 1,4-dioxane) at 20° C. under N₂ and stirred for 1 h. The reaction mixture was concentrated under reduced pressure to give 6-((4-fluoropiperidin-4-yl)methyl)nicotinonitrile hydrochloride (40 mg), which was carried directly into the next step.

Step 6

To a solution of [2,4′-bipyridine]-3-carboxylic acid (37.4 mg, 0.187 mmol) and HATU (88.9 mg, 0.234 mmol) in DMF (2 mL) was added DIPEA (0.135 mL, 0.780 mmol, 0.74 g/mL) at 20° C. and stirred for 15 min. 6-((4-fluoropiperidin-4-yl)methyl)nicotinonitrile hydrochloride (40 mg, 0.156 mmol) in DMF (2 mL) was added slowly into the mixture. The mixture was stirred at 20° C. for 3 h. The reaction mixture was poured into H₂O (10 mL) and stirred for 20 min. The aqueous phase was extracted with DCM (3×10 mL). The combined organic phase was washed with saturated brine (2×20 mL), dried over anhydrous Na₂SO₄, filtered, and concentrated. Purification by flash column (0-5% MeOH in DCM) and further purification by Prep-HPLC (Column: Phenomenex C18 80*40 mm*3 um; Condition: water (NH₃H₂O)-ACN; Begin B: 20; End B: 50; Gradient Time(min): 8; 100% B Hold Time(min): 2; FlowRate(ml/min): 30; Injections: 5) provided 6-((1-([2,4′-bipyridine]-3-carbonyl)-4-fluoropiperidin-4-yl)methyl) nicotinonitrile (15 mg, 23%).

¹H NMR (400 MHz, CDCl₃) δ_{H} 8.79 (dd, J=1.6, 4.8 Hz, 2H), 8.71 (m, 2H), 7.87 (dd, J=2.0, 8.4 Hz, 1H), 7.75 (m, 2H), 7.63 (s, 1H), 7.43 (dd, J=4.8, 8.0 Hz, 1H), 7.38-7.20 (m, 1H), 4.63-4.48 (m, 1H), 3.28-2.52 (m, 5H), 1.85-1.75 (m, 2H), 1.51-1.27 (m, 2H). ¹⁹F NMR (376.5 MHz, CDCl₃). δ_{F} −162.099. LCMS purity 99%, MS ESI calcd. For C₂₃H₂₀FN₅O [M+H]⁺ 402.2, found 402.2.

### Example 100. Synthesis of 5-(4-((5-chloropyridin-2-yl)methyl)-4-fluoropiperidine-1-carbonyl)-6-(pyrimidin-4-yl)picolinonitrile (Cmpd 98)

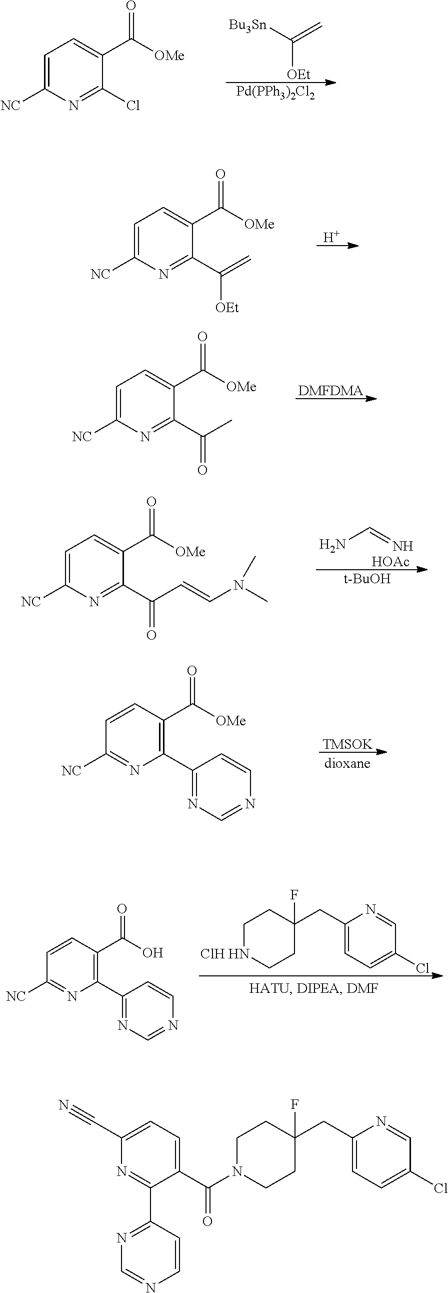

Step 1

To a solution of methyl 2-chloro-6-cyanonicotinate (5 g, 25.4 mmol) in toluene (50 mL) was added tributyl(1-ethoxyvinyl)stannane (10.9 g, 30.4 mmol) at 20° C. under N₂. The mixture was stirred at 20° C. for 15 min. Then Pd(PPh₃)₂Cl₂ (891 mg, 1.27 mmol) was added and the mixture was stirred for another 5 min at 20° C. The reaction mixture was heated to 100° C. for 16 h and then was poured into aq. KF (10 g in 100 mL water) and extracted with EtOAc (2×100 mL). The combined organic extracts were dried over anhydrous Na₂SO₄, filtered, and concentrated to afford methyl 6-cyano-2-(1-ethoxyvinyl)nicotinate (6 g), which was used directly in the next step without further purification.

¹H NMR (400 MHz, CDCl₃) δ_{H} 7.94 (d, J=7.6 Hz, 1H), 7.66 (d, J=7.6 Hz, 1H), 5.21 (d, J=2.8 Hz, 1H), 4.52 (d, J=2.8 Hz, 1H), 3.94-3.87 (q, 3H), 1.34 (t, J=7.2 Hz, 3H).

Step 2

To a solution of methyl 6-cyano-2-(1-ethoxyvinyl)nicotinate (3 g, 12.1 mmol) in acetone (50 mL) was added HCl (29.0 mL, 72.6 mmol, 2.5 M in water) at 20° C. and stirred for 2 h. The volatiles were removed under reduced pressure. The mixture was poured into NaHCO₃ (200 mL, aq.). The reaction mixture was extracted with DCM (2×100 mL). The combined organic layers were washed with brine (200 mL), dried over Na₂SO₄, filtered, and concentrated. Purification by flash column (0-40% EtOAc in PE) provided methyl 2-acetyl-6-cyanonicotinate (2.4 g).

¹H NMR (400 MHz, CDCl₃) δ_{H} 8.13 (d, J=8.0 Hz, 1H), 7.87 (d, J=8.0 Hz, 1H), 3.95 (s, 3H), 2.71 (s, 3H).

Step 3

To a solution of methyl 2-acetyl-6-cyanonicotinate (2.4 g, 11.7 mmol) in MeCN (30 mL) was added DMF-DMA (4.18 g, 35.1 mmol) at 20° C., and the mixture was stirred at 80° C. for 3 h. The reaction mixture was concentrated to afford methyl (E)-6-cyano-2-(3-(dimethylamino) acryloyl)nicotinate (3.2 g).

Step 4

To a mixture of methyl (E)-6-cyano-2-(3-(dimethylamino)acryloyl)nicotinate (1 g, 3.85 mmol) in t-BuOH (10 mL) was added DIPEA (13.4 mL, 77.0 mmol, 0.74 g/mL), acetic acid, and methanimidamide (5.00 g, 48.1 mmol) at 25° C., and the mixture was stirred at 120° C. for 16 h. The reaction mixture was poured into H₂O (50 mL) and stirred for 10 min. The aqueous phase was extracted with EtOAc (3×30 mL). The combined organic phase was washed with saturated brine (2×50 mL), dried over anhydrous Na₂SO₄, filtered, and concentrated. Purification by flash column (0-50% EtOAc in PE) provided methyl 6-cyano-2-(pyrimidin-4-yl)nicotinate (80 mg).

¹H NMR (400 MHz, CDCl₃) δ_{H} 9.25 (d, J=1.2 Hz, 1H), 8.97 (d, J=5.2 Hz, 1H), 8.22 (dd, J=1.6, 5.2 Hz, 1H), 8.12 (d, J=8.0 Hz, 1H), 7.86 (d, J=7.6 Hz, 1H), 3.87 (s, 3H).

Step 5

To a solution of methyl 6-cyano-2-(pyrimidin-4-yl)nicotinate (80 mg, 0.333 mmol) in dioxane (5 mL) was added TMSOK (51.1 mg, 0.399 mmol) at 25° C. and the mixture was stirred at 25° C. for 2 h. The reaction mixture was poured into citric acid monohydrate (10 mL) and adjusted to a pH of 5-6. The mixture was extracted with DCM (10 mL×3). The combined organic layers were dried over sodium sulfate, filtered, and concentrated to give 6-cyano-2-(pyrimidin-4-yl)nicotinic acid (50 mg).

¹H NMR (400 MHz, MeOD) δ_{H} 9.21 (d, J=1.2 Hz, 1H), 8.98 (d, J=5.2 Hz, 1H), 8.35 (d, J=8.0 Hz, 1H), 8.14 (dd, J=1.6, 5.2 Hz, 1H), 8.09 (d, J=8.0 Hz, 1H).

Step 6

To a solution of 6-cyano-2-(pyrimidin-4-yl)nicotinic acid (50 mg, 0.221 mmol) and HATU (125 mg, 0.331 mmol) in DMF (5 mL) was added DIPEA (0.191 mL, 1.10 mmol, 0.74 g/mL) at 20° C. 5-chloro-2-[(4-fluoropiperidin-4-yl)methyl]pyridine hydrochloride (52.4 mg, 0.198 mmol) in DMF (5 mL) was added slowly into the mixture. The mixture was stirred at 20° C. for 16 h. The reaction mixture was poured into H₂O (10 mL) and stirred for 10 min. The aqueous phase was extracted with DCM (3×10 mL). The combined organic phase was washed with saturated brine (2×20 mL), dried over anhydrous Na₂SO₄, filtered, and concentrated. Purification by flash column (0-5% MeOH in DCM) and further purification by Prep-HPLC (Column: Phenomenex C18 80*40 mm*3 um; Condition: water (NH₃H₂O)-ACN; Begin B: 34; End B: 64; Gradient Time(min): 8; 100% B Hold Time(min): 2.2; Flow Rate (ml/min): 30; Injections: 4) provided 5-(4-((5-chloropyridin-2-yl)methyl)-4-fluoropiperidine-1-carbonyl)-6-(pyrimidin-4-yl)picolinonitrile (7 mg, 7%).

¹H NMR (400 MHz, CDCl₃) δ_{H} 9.30-8.89 (m, 2H), 8.49-8.47 (m, 1H), 8.28-8.26 (m, 1H), 7.88-7.77 (m, 2H), 7.68-7.60 (m, 1H), 7.22 (t, J=7.2 Hz, 1H), 4.68-4.49 (m, 1H), 3.50-3.32 (m, 1H), 3.25-3.19 (m, 1H), 3.19-3.07 (m, 3H), 2.06-1.89 (m, 2H), 1.76-1.64 (m, 2H). ¹⁹F NMR (376.5 MHz, CDCl₃). δ_{F} −160.091. LC-ELSD/MS purity 99%, MS ESI calcd. For C₂₂H₁₈ClFN₆O [M+H]+ 437.1, found 437.1.

### Example 101. Exemplary CYP46A1 Enzyme Assay

Briefly, in a 384-well plate format, 10 μL per well of an enzyme-substrate mixture of CYP46A1 (5 μM final concentration) and Testosterone (10 mM final concentration) were dispensed to wells containing test compound. For CYP46A1 titration, 5 μL per well of serially diluted CYP46A1 (concentrations including 10 μM, 5 μM, and 2.5 μM) and Testosterone (concentration of 10 mM) were dispensed. For Testosterone titration, 5 μL per well of serially diluted Testosterone (concentrations including 10 μM, 5 μM, and 2.5 μM) and CYP46A1 (concentration of 5 μM) were dispensed. The plates were centrifuged at 1000 rpm for 30 seconds, and then sealed and incubated at 37° C. for 30 minutes. The plate was removed from the incubator, and then 10 μL per well of NADPH-generating system were dispensed to initiate the reaction. The plates were incubated at 37° C. for 15 minutes, added with 80 μL per well of 100% methanol consisting 1 ng/ml diclofenac as internal standard, and then transferred for HPLC-MS.

IC₅₀ values for exemplary compounds were also obtained with the assay described above, as shown in Table 2. In Table 2, A indicates a CYP46A1 IC₅₀<0.1 μM, B indicates a CYP46A1 IC₅₀ (μM) of 0.1 μM to <1.0 μM, and C indicates a CYP46A1 IC₅₀ (μM) of ≥1.0 μM

TABLE 2
CYP46A1 inhibitory activity data for exemplary compounds.

| Compound | | |
|---|---|---|
| No. | Structure | IC₅₀ |
| 1 | 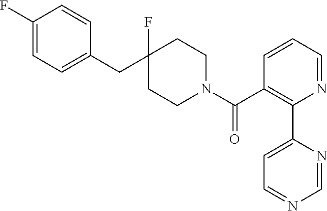 [SMILES: O=C(c1cccnc1-c1ccncn1)N1CCC(F)(Cc2ccc(F)cc2)CC1] | A |
| 4 | 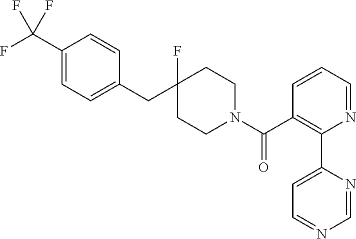 [SMILES: O=C(c1cccnc1-c1ccncn1)N1CCC(F)(Cc2ccc(C(F)(F)F)cc2)CC1] | A |
| 11 | 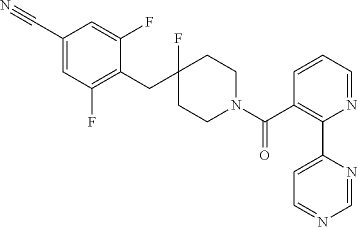 [SMILES: N#Cc1cc(F)c(CC2(F)CCN(C(=O)c3cccnc3-c3ccncn3)CC2)c(F)c1] | A |
| 2 | 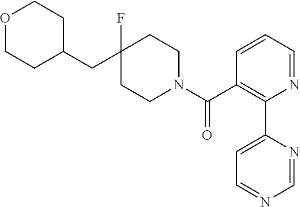 [SMILES: O=C(c1cccnc1-c1ccncn1)N1CCC(F)(CC2CCOCC2)CC1] | C |
| 3 | 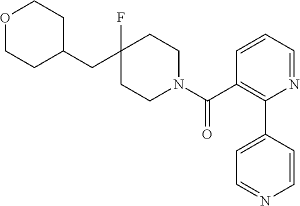 [SMILES: O=C(c1cccnc1-c1ccncc1)N1CCC(F)(CC2CCOCC2)CC1] | C |
| 5 | 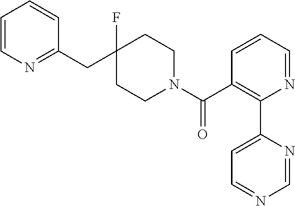 [SMILES: O=C(c1cccnc1-c1ccncn1)N1CCC(F)(Cc2ccccn2)CC1] | A |
| 6 | 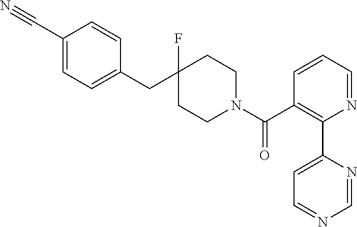 [SMILES: N#Cc1ccc(CC2(F)CCN(C(=O)c3cccnc3-c3ccncn3)CC2)cc1] | A |
| 7 | 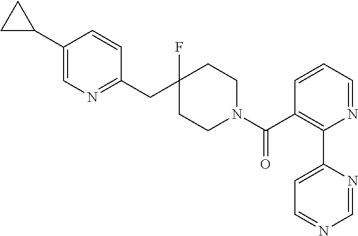 [SMILES: O=C(c1cccnc1-c1ccncn1)N1CCC(F)(Cc2ccc(C3CC3)cn2)CC1] | A |
| 9 | 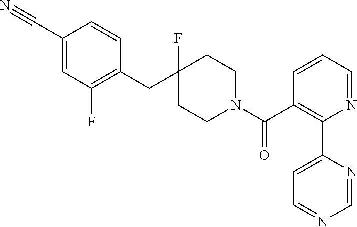 [SMILES: N#Cc1ccc(CC2(F)CCN(C(=O)c3cccnc3-c3ccncn3)CC2)c(F)c1] | A |
| 10 | 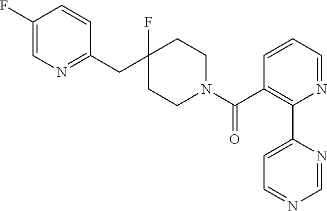 [SMILES: O=C(c1cccnc1-c1ccncn1)N1CCC(F)(Cc2ccc(F)cn2)CC1] | B |
| 12 | 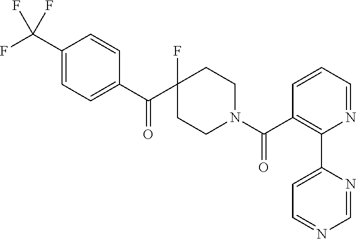 [SMILES: O=C(c1cccnc1-c1ccncn1)N1CCC(F)(C(=O)c2ccc(C(F)(F)F)cc2)CC1] | B |
| 15 | 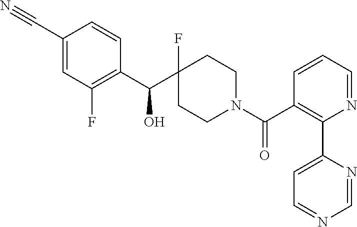 [SMILES: N#Cc1ccc([C@H](O)C2(F)CCN(C(=O)c3cccnc3-c3ccncn3)CC2)c(F)c1] | A |
| 16 | 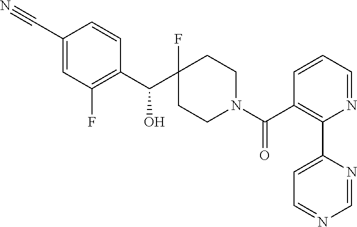 [SMILES: N#Cc1ccc([C@@H](O)C2(F)CCN(C(=O)c3cccnc3-c3ccncn3)CC2)c(F)c1] | B |
| 17 | 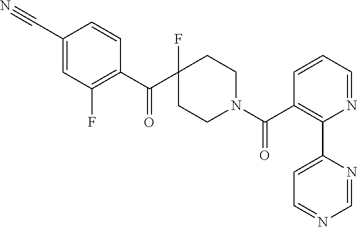 [SMILES: N#Cc1ccc(C(=O)C2(F)CCN(C(=O)c3cccnc3-c3ccncn3)CC2)c(F)c1] | B |
| 13 | 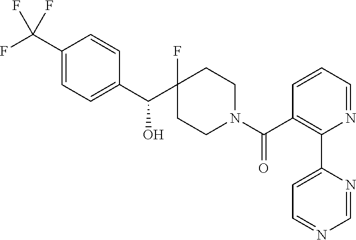 [SMILES: O=C(c1cccnc1-c1ccncn1)N1CCC(F)([C@H](O)c2ccc(C(F)(F)F)cc2)CC1] | A |
| 14 | 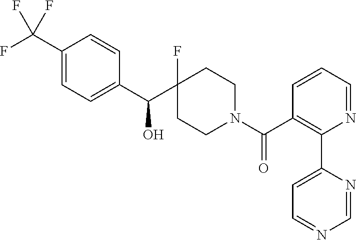 [SMILES: O=C(c1cccnc1-c1ccncn1)N1CCC(F)([C@@H](O)c2ccc(C(F)(F)F)cc2)CC1] | A |
| 18 | 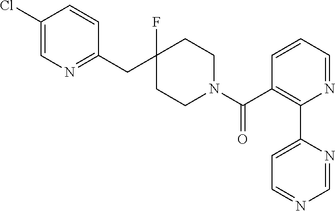 [SMILES: O=C(c1cccnc1-c1ccncn1)N1CCC(F)(Cc2ccc(Cl)cn2)CC1] | A |
| 19 | 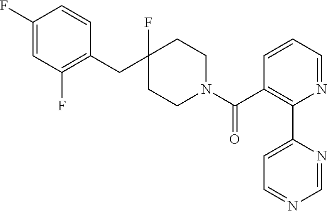 [SMILES: O=C(c1cccnc1-c1ccncn1)N1CCC(F)(Cc2ccc(F)cc2F)CC1] | A |
| 20 | 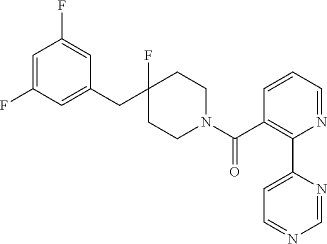 [SMILES: O=C(c1cccnc1-c1ccncn1)N1CCC(F)(Cc2cc(F)cc(F)c2)CC1] | A |
| 21 | 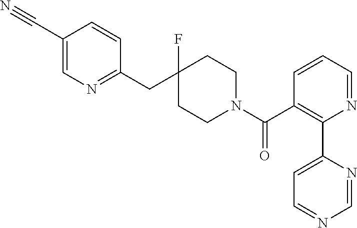 [SMILES: N#Cc1ccc(CC2(F)CCN(C(=O)c3cccnc3-c3ccncn3)CC2)nc1] | B |
| 22 | 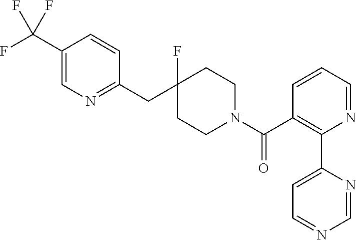 [SMILES: O=C(c1cccnc1-c1ccncn1)N1CCC(F)(Cc2ccc(C(F)(F)F)cn2)CC1] | A |
| 23 | 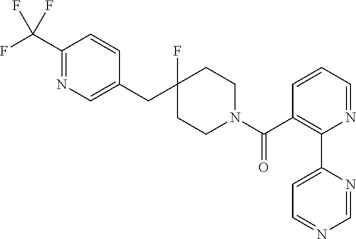 [SMILES: O=C(c1cccnc1-c1ccncn1)N1CCC(F)(Cc2ccc(C(F)(F)F)nc2)CC1] | A |
| 24 | 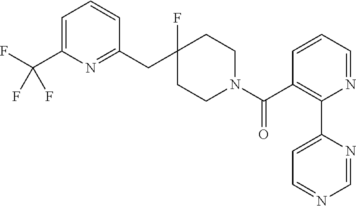 [SMILES: O=C(c1cccnc1-c1ccncn1)N1CCC(F)(Cc2cccc(C(F)(F)F)n2)CC1] | A |
| 25 | 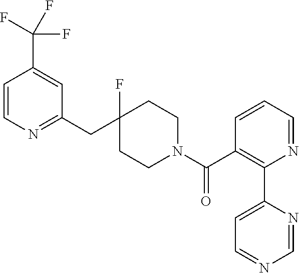 [SMILES: O=C(c1cccnc1-c1ccncn1)N1CCC(F)(Cc2cc(C(F)(F)F)ccn2)CC1] | B |
| 26 | 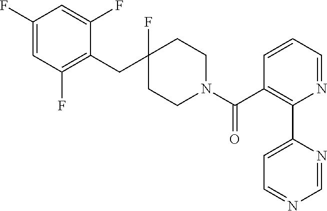 [SMILES: O=C(c1cccnc1-c1ccncn1)N1CCC(F)(Cc2c(F)cc(F)cc2F)CC1] | A |
| 27 | 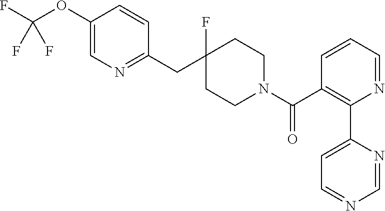 [SMILES: O=C(c1cccnc1-c1ccncn1)N1CCC(F)(Cc2ccc(OC(F)(F)F)cn2)CC1] | B |
| 28 | 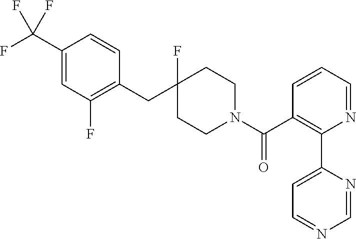 [SMILES: O=C(c1cccnc1-c1ccncn1)N1CCC(F)(Cc2ccc(C(F)(F)F)cc2F)CC1] | A |
| 29 | 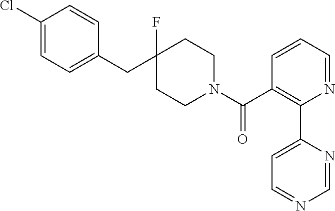 [SMILES: O=C(c1cccnc1-c1ccncn1)N1CCC(F)(Cc2ccc(Cl)cc2)CC1] | A |
| 30 | 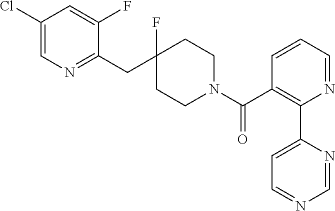 [SMILES: O=C(c1cccnc1-c1ccncn1)N1CCC(F)(Cc2ncc(Cl)cc2F)CC1] | B |
| 32 | 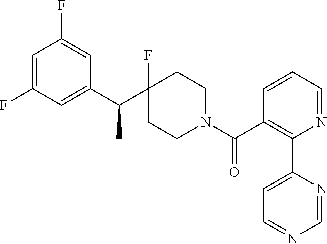 [SMILES: C[C@@H](c1cc(F)cc(F)c1)C1(F)CCN(C(=O)c2cccnc2-c2ccncn2)CC1] | A |
| 31 | 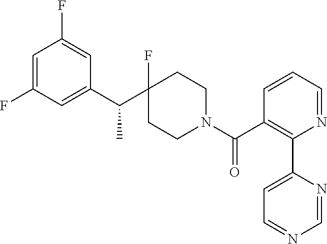 [SMILES: C[C@H](c1cc(F)cc(F)c1)C1(F)CCN(C(=O)c2cccnc2-c2ccncn2)CC1] | A |
| 33 | 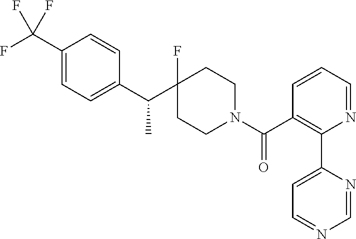 [SMILES: C[C@H](c1ccc(C(F)(F)F)cc1)C1(F)CCN(C(=O)c2cccnc2-c2ccncn2)CC1] | A |
| 34 | 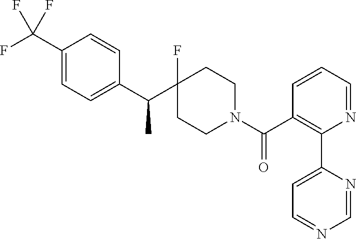 [SMILES: C[C@@H](c1ccc(C(F)(F)F)cc1)C1(F)CCN(C(=O)c2cccnc2-c2ccncn2)CC1] | A |
| 8 | 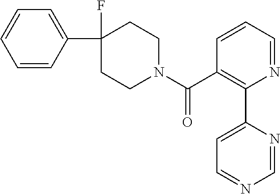 [SMILES: O=C(c1cccnc1-c1ccncn1)N1CCC(F)(c2ccccc2)CC1] | C |
| 35 | 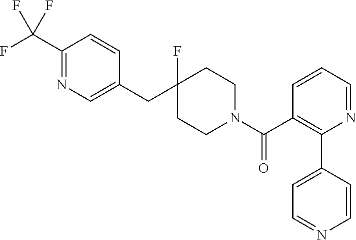 [SMILES: O=C(c1cccnc1-c1ccncc1)N1CCC(F)(Cc2ccc(C(F)(F)F)nc2)CC1] | A |
| 36 | 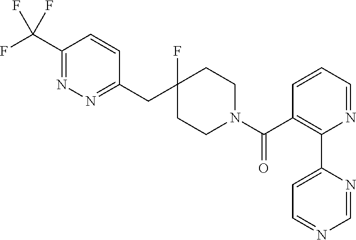 [SMILES: O=C(c1cccnc1-c1ccncn1)N1CCC(F)(Cc2ccc(C(F)(F)F)nn2)CC1] | B |
| 37 | 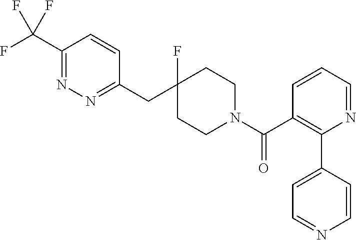 [SMILES: O=C(c1cccnc1-c1ccncc1)N1CCC(F)(Cc2ccc(C(F)(F)F)nn2)CC1] | B |
| 38 | 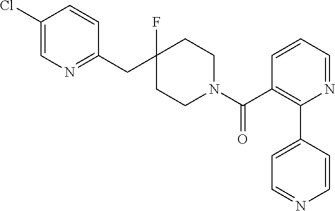 [SMILES: O=C(c1cccnc1-c1ccncc1)N1CCC(F)(Cc2ccc(Cl)cn2)CC1] | A |
| 39 | 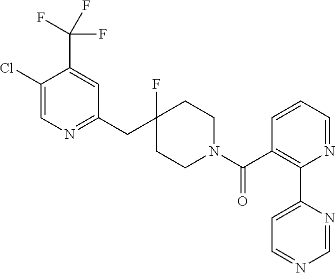 [SMILES: O=C(c1cccnc1-c1ccncn1)N1CCC(F)(Cc2cc(C(F)(F)F)c(Cl)cn2)CC1] | A |
| 40 | 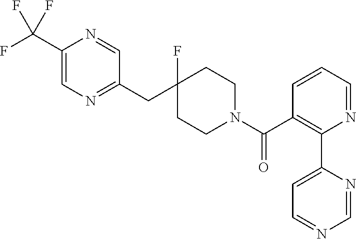 [SMILES: O=C(c1cccnc1-c1ccncn1)N1CCC(F)(Cc2cnc(C(F)(F)F)cn2)CC1] | B |
| 41 | 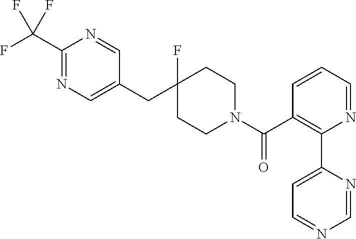 [SMILES: O=C(c1cccnc1-c1ccncn1)N1CCC(F)(Cc2cnc(C(F)(F)F)nc2)CC1] | B |
| 42 | 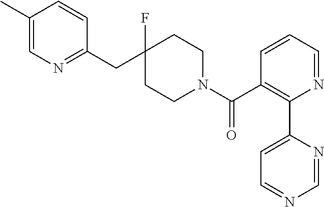 [SMILES: Cc1ccc(CC2(F)CCN(C(=O)c3cccnc3-c3ccncn3)CC2)nc1] | B |
| 43 | 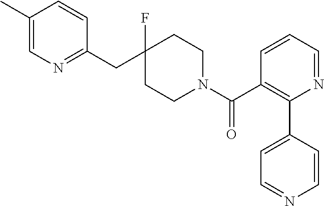 [SMILES: Cc1ccc(CC2(F)CCN(C(=O)c3cccnc3-c3ccncc3)CC2)nc1] | A |
| 44 | 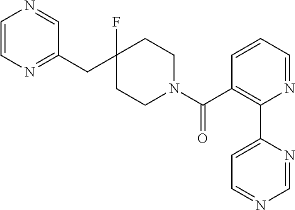 [SMILES: O=C(c1cccnc1-c1ccncn1)N1CCC(F)(Cc2cnccn2)CC1] | B |
| 45 | 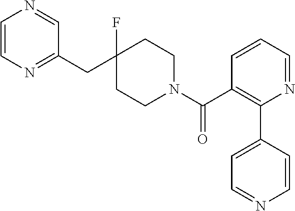 [SMILES: O=C(c1cccnc1-c1ccncc1)N1CCC(F)(Cc2cnccn2)CC1] | C |
| 46 | 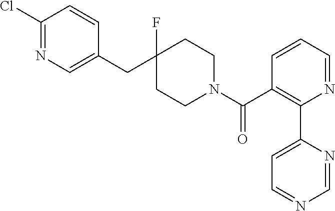 [SMILES: O=C(c1cccnc1-c1ccncn1)N1CCC(F)(Cc2ccc(Cl)nc2)CC1] | A |
| 47 | 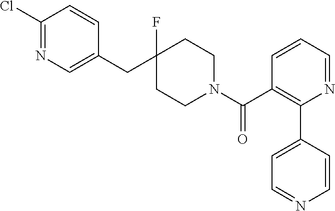 [SMILES: O=C(c1cccnc1-c1ccncc1)N1CCC(F)(Cc2ccc(Cl)nc2)CC1] | A |
| 48 | 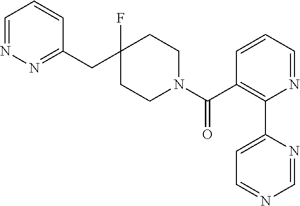 [SMILES: O=C(c1cccnc1-c1ccncn1)N1CCC(F)(Cc2cccnn2)CC1] | B |
| 49 | 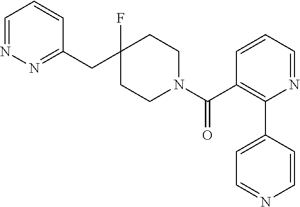 [SMILES: O=C(c1cccnc1-c1ccncc1)N1CCC(F)(Cc2cccnn2)CC1] | B |
| 50 | 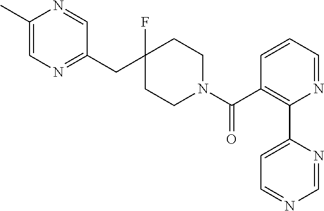 [SMILES: Cc1cnc(CC2(F)CCN(C(=O)c3cccnc3-c3ccncn3)CC2)cn1] | B |
| 51 | 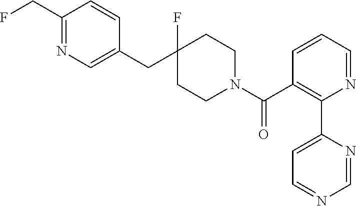 [SMILES: O=C(c1cccnc1-c1ccncn1)N1CCC(F)(Cc2ccc(CF)nc2)CC1] | A |
| 52 | 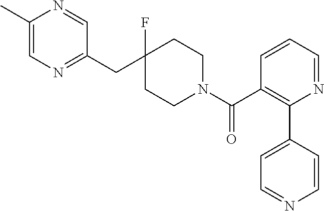 [SMILES: Cc1cnc(CC2(F)CCN(C(=O)c3cccnc3-c3ccncc3)CC2)cn1] | A |
| 53 | 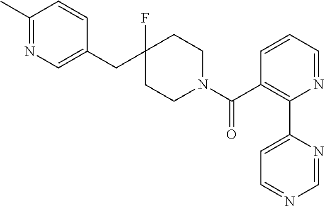 [SMILES: Cc1ccc(CC2(F)CCN(C(=O)c3cccnc3-c3ccncn3)CC2)cn1] | B |
| 54 | 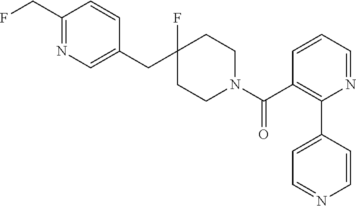 [SMILES: O=C(c1cccnc1-c1ccncc1)N1CCC(F)(Cc2ccc(CF)nc2)CC1] | A |
| 55 | 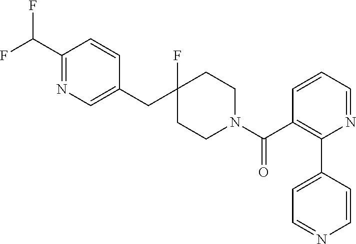 [SMILES: O=C(c1cccnc1-c1ccncc1)N1CCC(F)(Cc2ccc(C(F)F)nc2)CC1] | A |
| 56 | 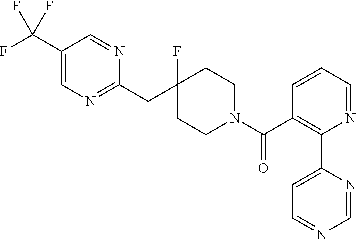 [SMILES: O=C(c1cccnc1-c1ccncn1)N1CCC(F)(Cc2ncc(C(F)(F)F)cn2)CC1] | C |
| 57 | 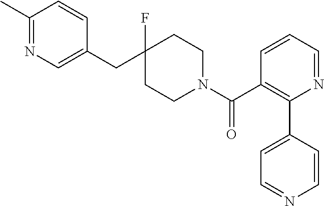 [SMILES: Cc1ccc(CC2(F)CCN(C(=O)c3cccnc3-c3ccncc3)CC2)cn1] | A |

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed:

1. A compound of Formula I: 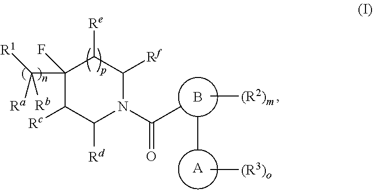
or a pharmaceutically acceptable salt thereof, wherein:
R¹ is selected from the group consisting of C₆-C₁₀ aryl, C₃-C₇ cycloalkyl, 3-7 membered heterocyclyl, and 5-10 membered heteroaryl, wherein R¹ is optionally substituted with one to four R⁴;
each of R^{a} and R^{b} is independently selected from the group consisting of H, halo, —CN, —OH, —NO₂, —N(R⁵)₂, C₁-C₆ alkyl, C₁-C₆ haloalkyl, C₁-C₆ alkoxy, and C₁-C₆ haloalkoxy; or R^{a} and R^{b} may form, together with the carbon to which they are attached, a C₃-C₇ cycloalkyl; or R^{a} and R^{b} taken together are oxo;
each of R^{c}, R^{d}, R^{e}, and R^{f} is independently selected from the group consisting of H, C₁-C₆ alkyl, C₁-C₆ haloalkyl, C₁-C₆ alkoxy, and C₁-C₆ haloalkoxy; or R^{c} and R^{e} may form, together with the carbons to which they are attached, a C₁-C₃ alkylene bridge; or R^{d} and R^{f} may form, together with the carbons to which they are attached, a C₁-C₃ alkylene bridge;
each R⁴ is independently selected from the group consisting of halo, —CN, —OH, —NO₂, —N(R⁵)₂, —S(O)₂R⁵, C₁-C₆ alkyl, C₁-C₆ haloalkyl, C₁-C₆ alkoxy, C₁-C₆ haloalkoxy, C₆-C₁₀ aryl, C₃-C₇ cycloalkyl, and 3-7 membered heterocyclyl;
each R⁵ is independently selected from H and C₁-C₆ alkyl;
each R² is independently selected from the group consisting of halo, —CN, —OH, —NO₂, —N(R⁵)₂, C₁-C₆ alkyl, C₃-C₇ cycloalkyl, C₁-C₆ haloalkyl, C₁-C₆ alkoxy, and C₁-C₆ haloalkoxy;
each R³ is independently selected from the group consisting of halo, —CN, —OH, —NO₂, —N(R⁵)₂, C₁-C₆ alkyl, C₁-C₆ haloalkyl, C₁-C₆ alkoxy, C₃-C₇ cycloalkyl, and C₁-C₆ haloalkoxy;
A is a 5-6 membered nitrogen-containing heteroaryl;
B is selected from C₆-C₁₀ aryl and 5-6 membered heteroaryl;
m is 0, 1, 2, or 3;
n is 0, 1, 2, 3, or 4;
o is 0, 1, 2, or 3; and
p is 0, 1, or 2;
provided that when n is 0, R¹ is not 4-cyanophenyl or 4-trifluomethylphenyl.

2. The compound of claim 1, wherein the compound of Formula I is not: 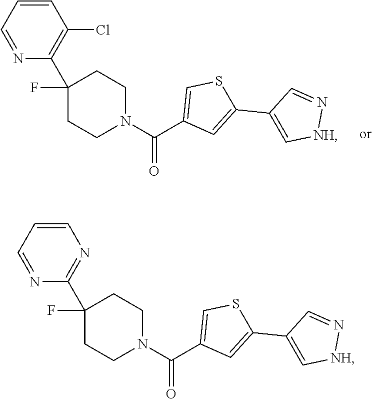 [SMILES: O=C(c1csc(-c2cn[nH]c2)c1)N1CCC(F)(c2ncccc2Cl)CC1 ; "or" ; O=C(c1csc(-c2cn[nH]c2)c1)N1CCC(F)(c2ncccn2)CC1]
or a pharmaceutically acceptable salt thereof.

3. The compound of claim 1, wherein the compound of Formula I is not: 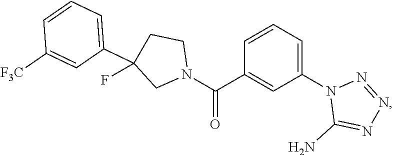 [SMILES: Nc1nnnn1-c1cccc(C(=O)N2CCC(F)(c3cccc(C(F)(F)F)c3)C2)c1]
or a pharmaceutically acceptable salt thereof.

4. The compound of any one of claims 1-3, wherein the compound of Formula I is a compound of Formula I-a-1: 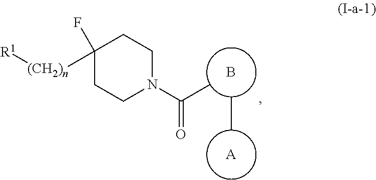
or a pharmaceutically acceptable salt thereof.

5. The compound of any one of claims 1-4, wherein R¹ is substituted C₆-C₁₀ aryl.

6. The compound of any one of claims 1-4, wherein R¹ is unsubstituted C₆-C₁₀ aryl.

7. The compound of any one of claims 1-4, wherein R¹ is 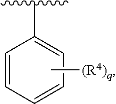
wherein each R⁴ is independently halo, —CN, —OH, —NO₂, —N(R⁵)₂, —S(O)₂R⁵, C₁-C₆ alkyl, C₁-C₆ haloalkyl, C₁-C₆ alkoxy, C₁-C₆ haloalkoxy, C₆-C₁₀ aryl, C₃-C₇ cycloalkyl, or 3-7 membered heterocyclyl; wherein each R⁵ is independently H or C₁-C₆ alkyl; and q is 0, 1, 2, or 3.

8. The compound of claim 7, wherein each R⁴ is independently halo, —CN, C₁-C₆ haloalkyl, and q is 0, 1, 2, or 3.

9. The compound of claim 7, wherein R¹ is

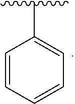 [SMILES: *c1ccccc1]

10. The compound of claim 7, wherein R¹ is

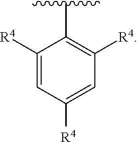

11. The compound of claim 7, wherein R¹ is

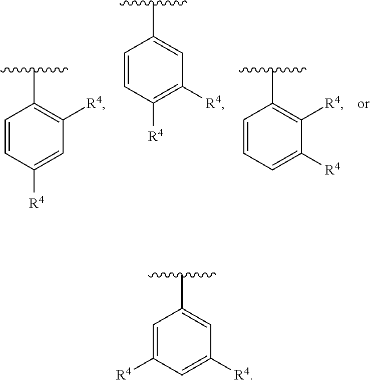

12. The compound of claim 7, wherein R¹ is

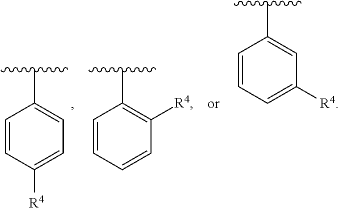

13. The compound of claim 10, wherein R¹ is

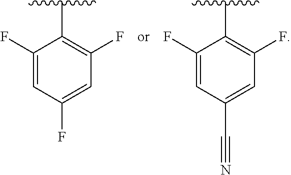 [SMILES: *c1c(F)cc(F)cc1F ; *c1c(F)cc(C#N)cc1F]

14. The compound of claim 10, wherein R¹ is

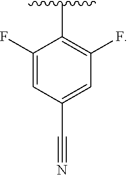 [SMILES: *c1c(F)cc(C#N)cc1F]

15. The compound of claim 11, wherein R¹ is

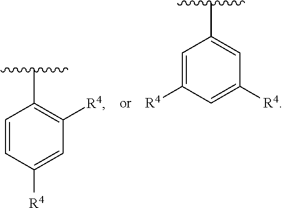

16. The compound of claim 15, wherein R¹ is

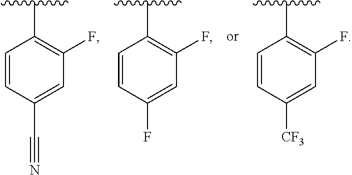

17. The compound of claim 15, wherein R¹ is

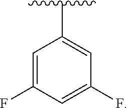 [SMILES: *c1cc(F)cc(F)c1]

18. The compound of claim 12, wherein R¹ is

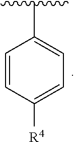

19. The compound of claim 18, wherein R¹ is:

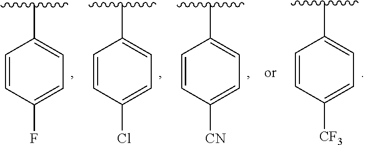

20. The compound of any one of claims 1-4, wherein R¹ is substituted 5-10 membered heteroaryl.

21. The compound of claim any one of claims 1-4, wherein R¹ is unsubstituted 5-10 membered heteroaryl.

22. The compound of claim 20 or 21, wherein R¹ is pyrrolyl, furanyl, thiophenyl, imidazolyl, pyrazolyl, oxazolyl, isoxazolyl, thiazolyl, isothiazolyl, triazolyl, oxadiazolyl, thiadiazolyl, pyridyl, pyridazinyl, pyrimidinyl, pyrazinyl, triazinyl, tetrazinyl, tetrazolyl, azocinyl, dithiazinyl, or oxazinyl.

23. The compound of claim 22, wherein R¹ is pyridyl, pyridinyl, pyridazinyl, pyrimidinyl, or pyrazinyl.

24. The compound of claim 23, wherein R¹ is 2-pyridyl, 3-pyridyl, 4-pyridyl, 2-pyrimidinyl, or 4-pyrimidinyl.

25. The compound of claim 20 or 21, wherein R¹ is 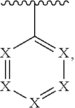
wherein each X is independently CH or N, wherein the H of CH is optionally substituted with one to four R⁴; wherein each R⁴ is independently halo, —CN, —OH, —NO₂, —N(R⁵)₂, C₁-C₆ alkyl, C₁-C₆ haloalkyl, C₁-C₆ alkoxy, C₁-C₆ haloalkoxy, C₆-C₁₀ aryl, C₃-C₇ cycloalkyl, or 3-7 membered heterocyclyl; and each R⁵ is independently H or C₁-C₆ alkyl.

26. The compound of claim 21, wherein R¹ is

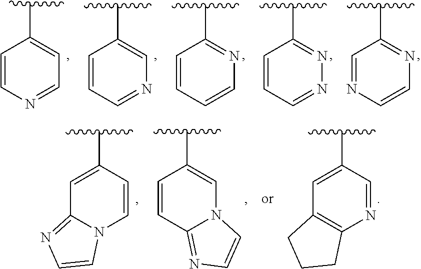

27. The compound of claim 21, wherein R¹ is

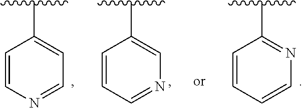

28. The compound of claim 21, wherein R¹ is

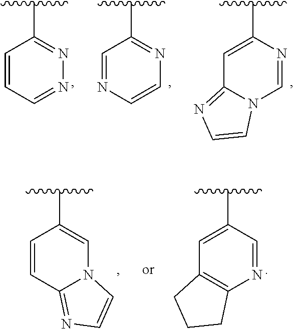

29. The compound of claim 27, wherein R¹ is

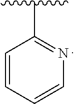 [SMILES: *c1ccccn1]

30. The compound of claim 20, wherein R¹ is:

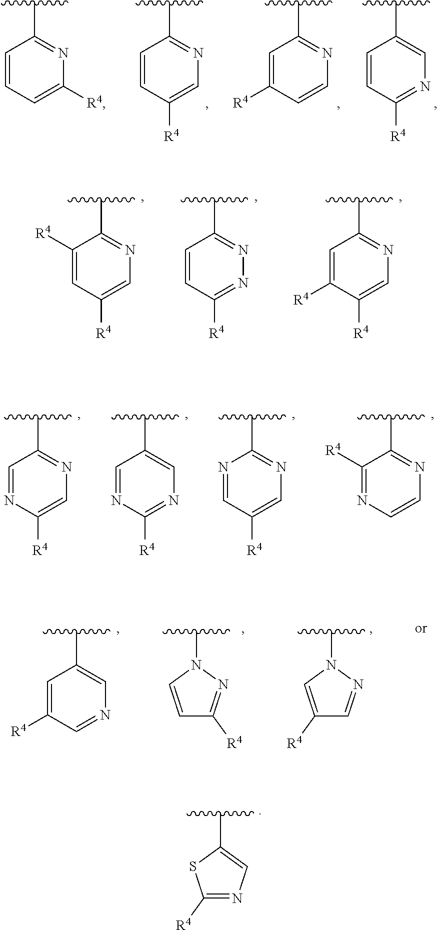

31. The compound of claim 30, wherein R¹ is:

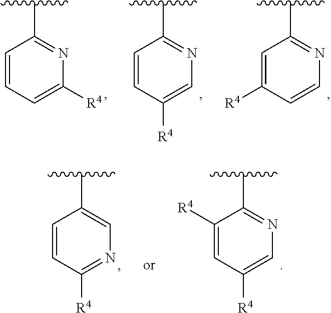

32. The compound of claim 30, wherein R¹ is:

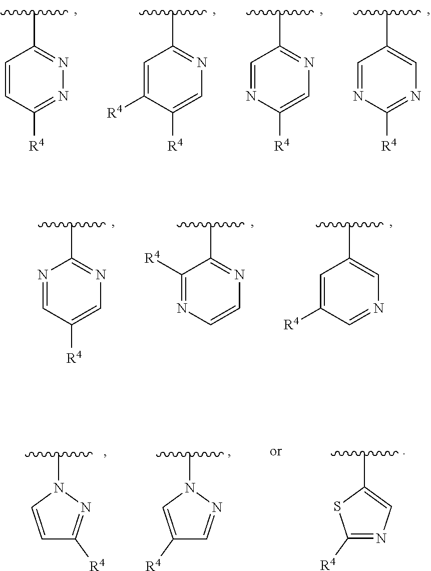

33. The compound of claim 30, wherein R¹ is:

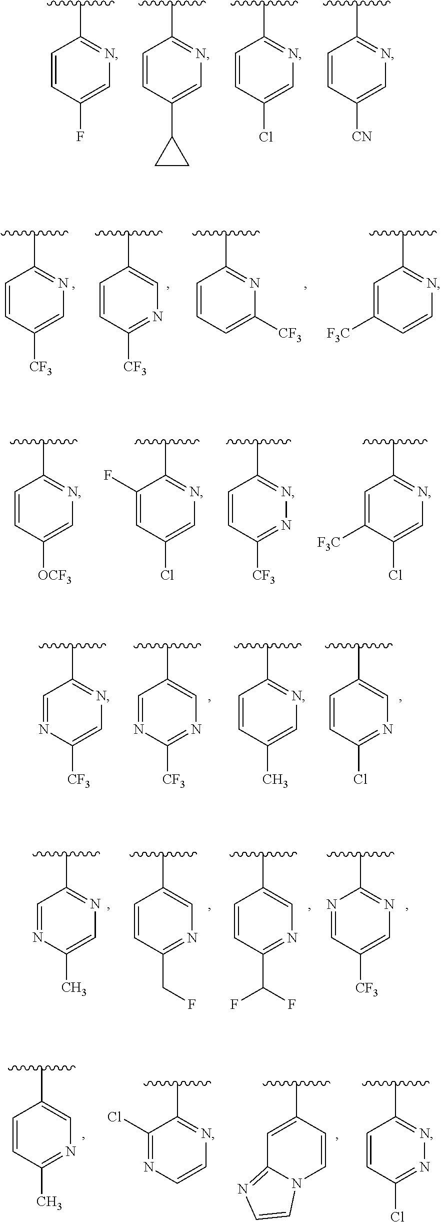

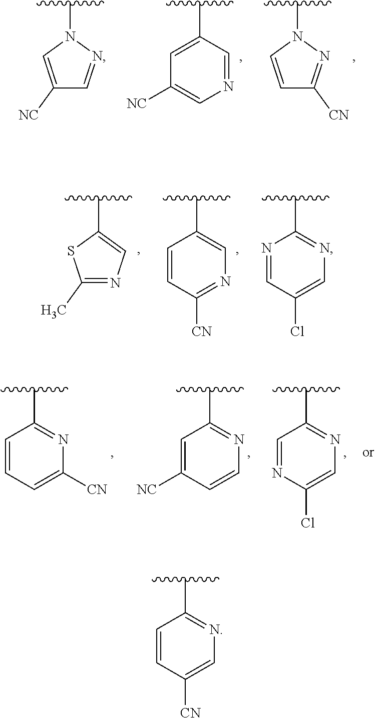

34. The compound of claim 33, wherein R¹ is:

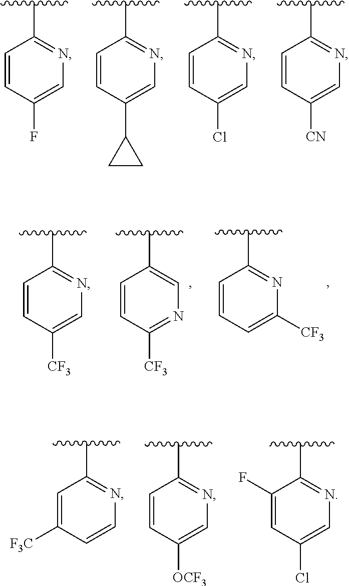

35. The compound of claim 33, wherein R¹ is:

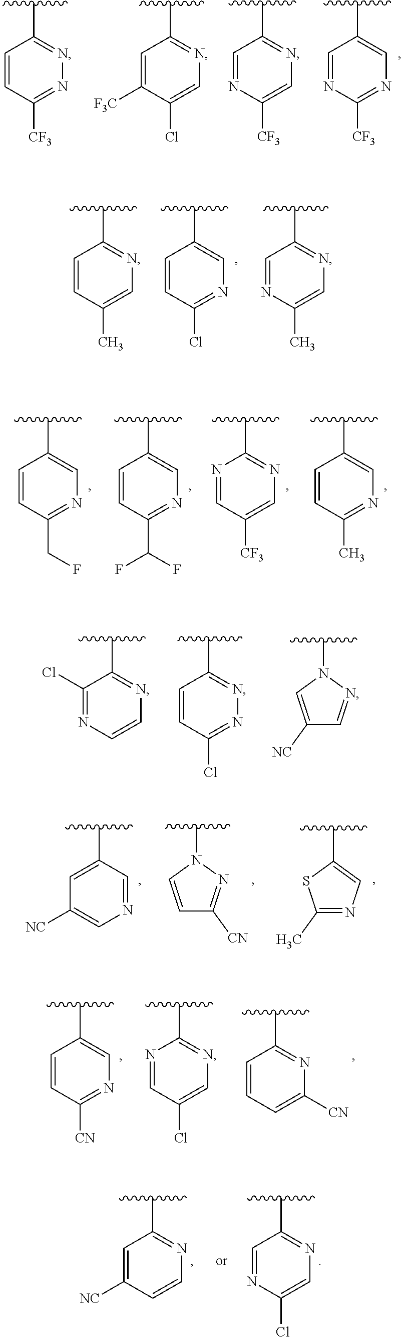

36. The compound of any one of claims 1-4, wherein R¹ is substituted 3-7 membered heterocyclyl.

37. The compound of any one of claims 1-4, wherein R¹ is unsubstituted 3-7 membered heterocyclyl.

38. The compound of claim 36 or 37, wherein R¹ is tetrahydrofuran, tetrahydropyran, pyrrolidine, piperidine, piperazine, dioxolane, dioxane, thiomorpholine, or dithiane.

39. The compound of claim 36 or 37, wherein R¹ is tetrahydrofuran or tetrahydropyran.

40. The compound of claim 36 or 37, wherein R¹ is

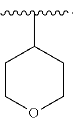 [SMILES: *C1CCOCC1]

41. The compound of any one of claims 1-40, wherein each R⁴ is independently halo, —CN, substituted C₁-C₆ alkyl, substituted C₁-C₆ alkoxy, or substituted C₃-C₇ cycloalkyl.

42. The compound of any one of claims 1-40, wherein each R⁴ is independently halo, —CN, unsubstituted C₁-C₆ alkyl, unsubstituted C₁-C₆ alkoxy, or unsubstituted C₃-C₇ cycloalkyl.

43. The compound of any one of claims 1-40, wherein each R⁴ is independently halo, —CN, C₁-C₆ alkyl, C₁-C₆ haloalkyl, C₁-C₆ alkoxy, C₁-C₆ haloalkoxy, or C₃-C₇ cycloalkyl.

44. The compound of claim 43, wherein each R⁴ is independently halo, —CN, —CH₃, —CF₃, —CH₂F, —CHF₂, —OCH₃, —OCF₃, or cyclopropyl.

45. The compound of claim 43, wherein each R⁴ is independently halo, —CN, —CF₃, —OCF₃, or cyclopropyl.

46. The compound of claim 43, wherein each R⁴ is independently halo, —CN, —CH₃, —CF₃, —CH₂F, or —CHF₂.

47. The compound of claim 43, wherein each R⁴ is independently Cl, F, Br, or I.

48. The compound of claim 47, wherein each R⁴ is independently Cl, or F.

49. The compound of any one of claims 1-48, wherein n is 4.

50. The compound of any one of claims 1-48, wherein n is 3.

51. The compound of any one of claims 1-48, wherein n is 2.

52. The compound of any one of claims 1-48, wherein n is 1.

53. The compound of any one of claims 1-48, wherein n is 0.

54. The compound of any one of claims 1-48, wherein n is 1, and R^{a} is C₁-C₆ alkyl and R^{b} is H.

55. The compound of any one of claims 1-48, wherein n is 1, and R^{a} is methyl and R^{b} is H.

56. The compound of any one of claims 1-48, wherein n is 1, R^{a} is —OH, and R^{b} is H.

57. The compound of any one of claims 1-48, wherein n is 1, and R^{a} and R^{b} are taken together to form an oxo.

58. The compound of any one of claims 1-48, wherein n is 1, and R^{a} and R^{b} are both H.

59. The compound of any one of claims 1-58, wherein p is 2.

60. The compound of any one of claims 1-58, wherein p is 1.

61. The compound of any one of claims 1-58, wherein p is 1, and R^{c}, R^{d}, R^{e}, and R^{f} are H.

62. The compound of any one of claims 1-58, wherein p is 1, R^{c} is methyl, and R^{d}, R^{e}, and R^{f} are H.

63. The compound of any one of claims 1-58, wherein p is 1, R^{c} and R^{e} are H, and R^{d} and R^{f} form together with the carbon to which they are attached, an C₁-C₃ alkylene bridge.

64. The compound of any one of claims 1-58, wherein p is 1, R^{d} and R^{f} are H, and R^{c} and R^{e} form together with the carbon to which they are attached, an C₁-C₃ alkylene bridge.

65. The compound of any one of claims 1-58, wherein p is 0.

66. The compound of any one of claims 1-58, wherein p is 0, and R^{c}, R^{d}, and R^{f} are H.

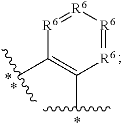

67. The compound of any one of claims 1-66, wherein B is; wherein each R⁶ is independently N or CR^{6a}, wherein R^{6a} is H or R²; and ** is the point of attachment to the carbonyl, and * is the point of attachment to A.

68. The compound of claim 67, wherein up to two R⁶ may be N and the other occurrences of R⁶ are CH.

69. The compound of any one of claims 1-68, wherein B is: 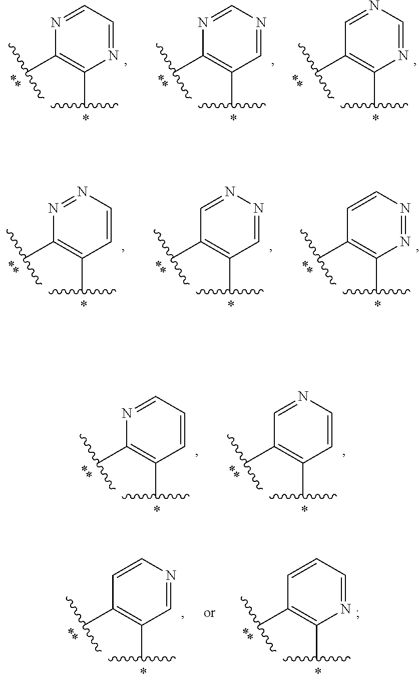
wherein ** is the point of attachment to a carbonyl, and * is the point of attachment to A.

70. The compound ofany one of claims 1-67, wherein B is 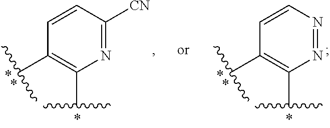 [SMILES: *c1ccc(C#N)nc1* ; *c1ccnnc1*]
wherein ** is the point of attachment to a carbonyl, and * is the point of attachment to A.

71. The compound of claim 70, wherein B is 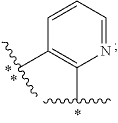 [SMILES: *c1cccnc1*]
wherein ** is the point of attachment to a carbonyl, and * is the point of attachment to A.

72. The compound of claim 70, wherein B is 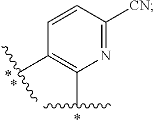 [SMILES: *c1ccc(C#N)nc1*]
wherein ** is the point of attachment to a carbonyl, and * is the point of attachment to A.

73. The compound of claim 70, wherein B is 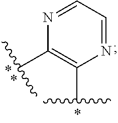 [SMILES: *c1nccnc1*]
wherein ** is the point of attachment to a carbonyl, and * is the point of attachment to A.

74. The compound of claim 70, wherein B is 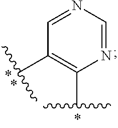 [SMILES: *c1cncnc1*]
wherein ** is the point of attachment to a carbonyl, and * is the point of attachment to A.

75. The compound of claim 70, wherein B is 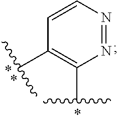 [SMILES: *c1ccnnc1*]
wherein ** is the point of attachment to a carbonyl, and * is the point of attachment to A.

76. The compound of any one of claims 1-66, wherein B is 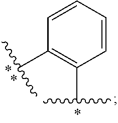 [SMILES: *c1ccccc1*]
wherein ** is the point of attachment to a carbonyl, and * is the point of attachment to A.

77. The compound of any one of claims 1-66, wherein m is 1.

78. The compound of any one of claims 1-66, wherein m is 0.

79. The compound of any one of claims 1-78, wherein A is pyridinyl, pyrrolyl, imidazolyl, pyrazolyl, pyridazinyl, pyrimidinyl, pyrazinyl, triazolyl, triazinyl, tetrazinyl, tetrazolyl, oxazolyl, isoxazolyl, or thiozolyl.

80. The compound of any one of claims 1-79, wherein A is pyridinyl oxazolyl, imidazolyl, triazolyl, pyridinyl, pyridazinyl, pyrimidinyl, or triazinyl.

81. The compound of any one of claims 1-80, wherein A is 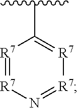
wherein each R⁷ is independently N or CH, wherein up to two R⁷ may be N and the other occurrences of R⁷ are CH, wherein the hydrogen of CH may be substituted with R³.

82. The compound of any one of claims 1-81, wherein A is:

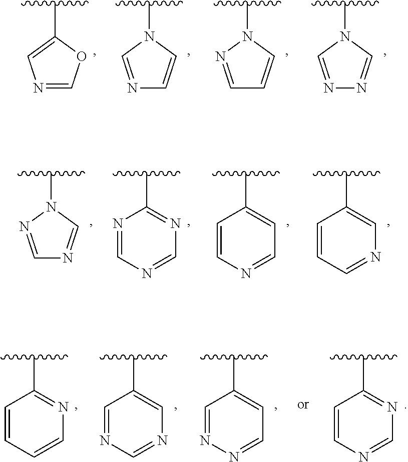

83. The compound of any one of claims 1-82, wherein A is:

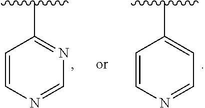 [SMILES: *c1ccncn1 ; *c1ccncc1]

84. The compound of any one of claims 1-83, wherein the compound is selected from the group consisting of: 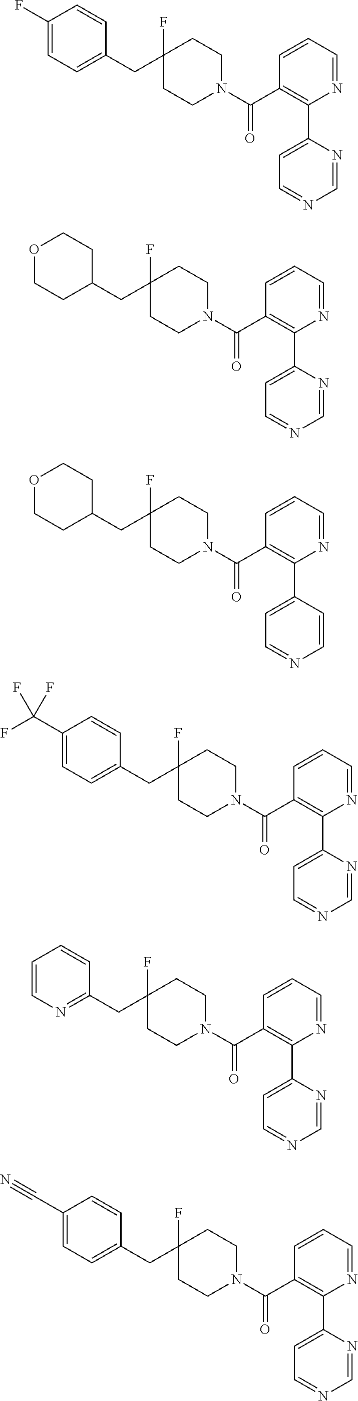 [SMILES: O=C(c1cccnc1-c1ccncn1)N1CCC(F)(Cc2ccc(F)cc2)CC1 ; O=C(c1cccnc1-c1ccncn1)N1CCC(F)(CC2CCOCC2)CC1 ; O=C(c1cccnc1-c1ccncc1)N1CCC(F)(CC2CCOCC2)CC1 ; O=C(c1cccnc1-c1ccncn1)N1CCC(F)(Cc2ccc(C(F)(F)F)cc2)CC1 ; O=C(c1cccnc1-c1ccncn1)N1CCC(F)(Cc2ccccn2)CC1 ; N#Cc1ccc(CC2(F)CCN(C(=O)c3cccnc3-c3ccncn3)CC2)cc1] 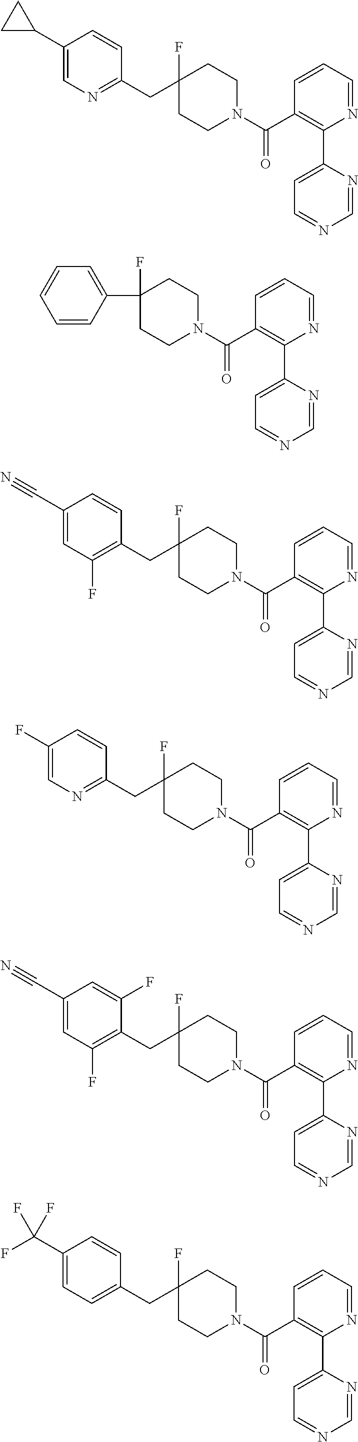 [SMILES: O=C(c1cccnc1-c1ccncn1)N1CCC(F)(Cc2ccc(C3CC3)cn2)CC1 ; O=C(c1cccnc1-c1ccncn1)N1CCC(F)(c2ccccc2)CC1 ; N#Cc1ccc(CC2(F)CCN(C(=O)c3cccnc3-c3ccncn3)CC2)c(F)c1 ; O=C(c1cccnc1-c1ccncn1)N1CCC(F)(Cc2ccc(F)cn2)CC1 ; N#Cc1cc(F)c(CC2(F)CCN(C(=O)c3cccnc3-c3ccncn3)CC2)c(F)c1 ; O=C(c1cccnc1-c1ccncn1)N1CCC(F)(Cc2ccc(C(F)(F)F)cc2)CC1] 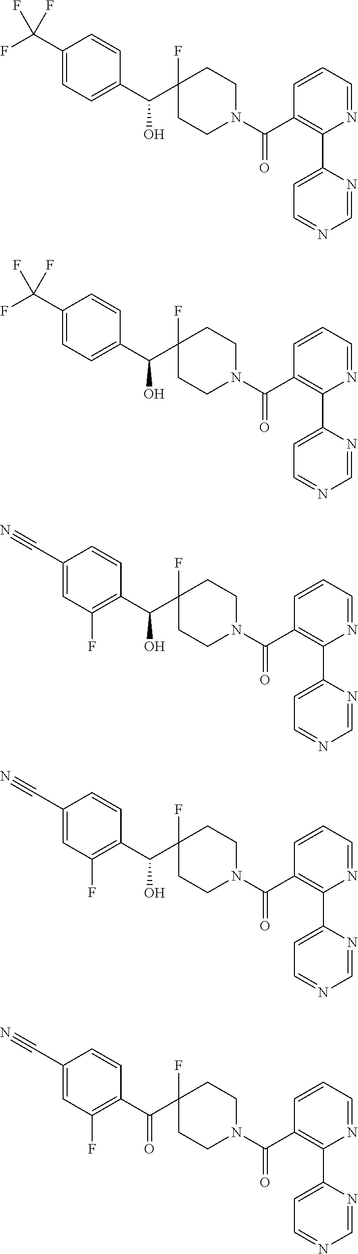 [SMILES: O=C(c1cccnc1-c1ccncn1)N1CCC(F)([C@H](O)c2ccc(C(F)(F)F)cc2)CC1 ; O=C(c1cccnc1-c1ccncn1)N1CCC(F)([C@@H](O)c2ccc(C(F)(F)F)cc2)CC1 ; N#Cc1ccc([C@H](O)C2(F)CCN(C(=O)c3cccnc3-c3ccncn3)CC2)c(F)c1 ; N#Cc1ccc([C@@H](O)C2(F)CCN(C(=O)c3cccnc3-c3ccncn3)CC2)c(F)c1 ; N#Cc1ccc(C(=O)C2(F)CCN(C(=O)c3cccnc3-c3ccncn3)CC2)c(F)c1] 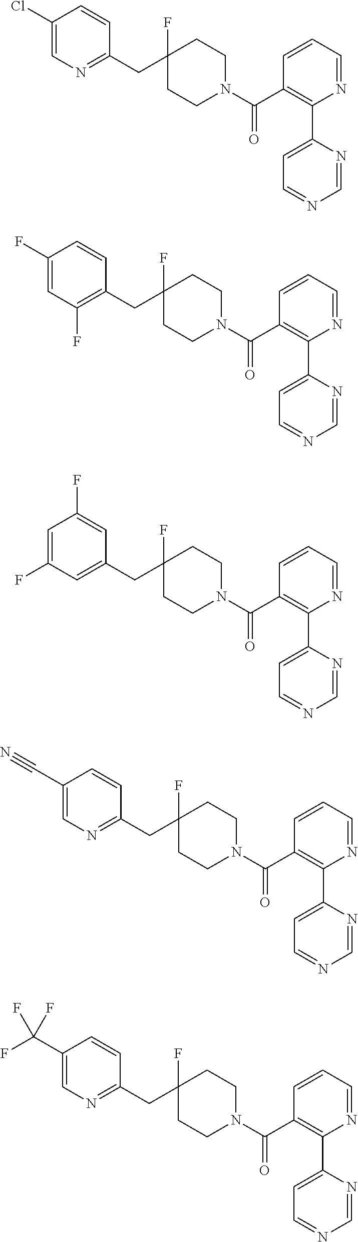 [SMILES: O=C(c1cccnc1-c1ccncn1)N1CCC(F)(Cc2ccc(Cl)cn2)CC1 ; O=C(c1cccnc1-c1ccncn1)N1CCC(F)(Cc2ccc(F)cc2F)CC1 ; O=C(c1cccnc1-c1ccncn1)N1CCC(F)(Cc2cc(F)cc(F)c2)CC1 ; N#Cc1ccc(CC2(F)CCN(C(=O)c3cccnc3-c3ccncn3)CC2)nc1 ; O=C(c1cccnc1-c1ccncn1)N1CCC(F)(Cc2ccc(C(F)(F)F)cn2)CC1] 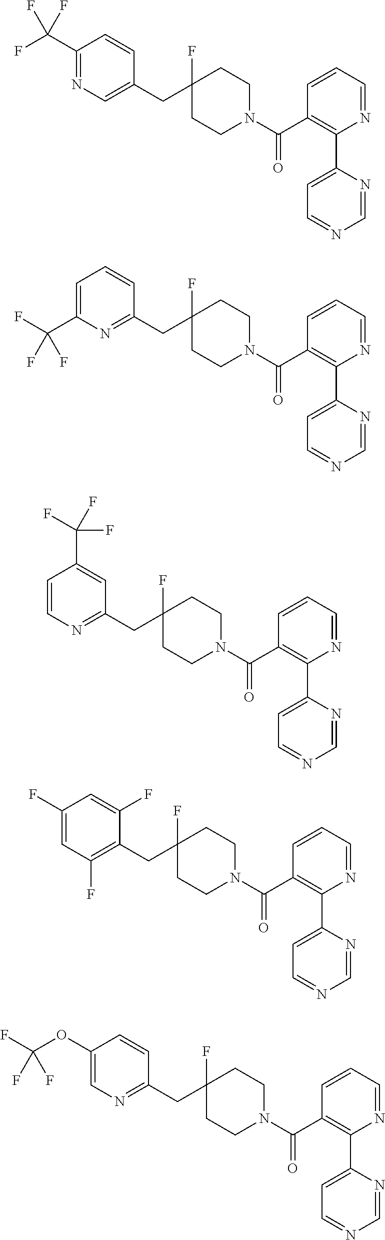 [SMILES: O=C(c1cccnc1-c1ccncn1)N1CCC(F)(Cc2ccc(C(F)(F)F)nc2)CC1 ; O=C(c1cccnc1-c1ccncn1)N1CCC(F)(Cc2cccc(C(F)(F)F)n2)CC1 ; O=C(c1cccnc1-c1ccncn1)N1CCC(F)(Cc2cc(C(F)(F)F)ccn2)CC1 ; O=C(c1cccnc1-c1ccncn1)N1CCC(F)(Cc2c(F)cc(F)cc2F)CC1 ; O=C(c1cccnc1-c1ccncn1)N1CCC(F)(Cc2ccc(OC(F)(F)F)cn2)CC1] 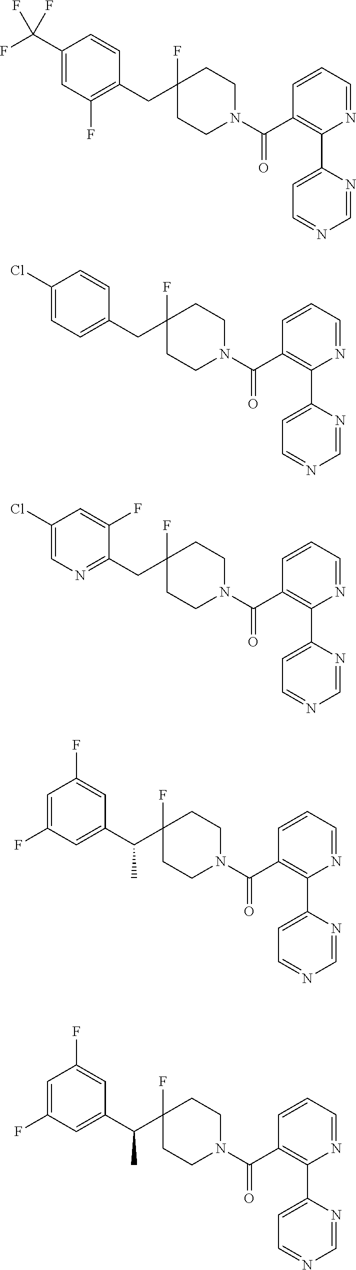 [SMILES: O=C(c1cccnc1-c1ccncn1)N1CCC(F)(Cc2ccc(C(F)(F)F)cc2F)CC1 ; O=C(c1cccnc1-c1ccncn1)N1CCC(F)(Cc2ccc(Cl)cc2)CC1 ; O=C(c1cccnc1-c1ccncn1)N1CCC(F)(Cc2ncc(Cl)cc2F)CC1 ; C[C@H](c1cc(F)cc(F)c1)C1(F)CCN(C(=O)c2cccnc2-c2ccncn2)CC1 ; C[C@@H](c1cc(F)cc(F)c1)C1(F)CCN(C(=O)c2cccnc2-c2ccncn2)CC1] 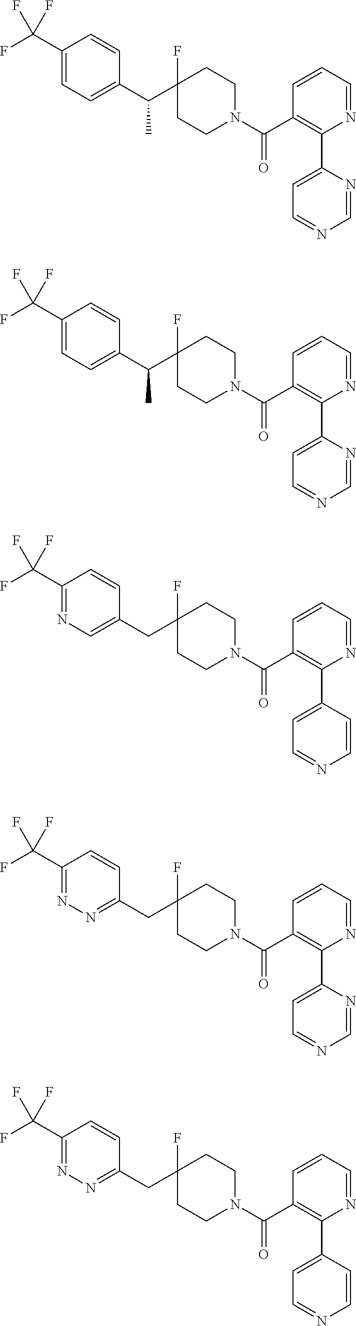 [SMILES: C[C@H](c1ccc(C(F)(F)F)cc1)C1(F)CCN(C(=O)c2cccnc2-c2ccncn2)CC1 ; C[C@@H](c1ccc(C(F)(F)F)cc1)C1(F)CCN(C(=O)c2cccnc2-c2ccncn2)CC1 ; O=C(c1cccnc1-c1ccncc1)N1CCC(F)(Cc2ccc(C(F)(F)F)nc2)CC1 ; O=C(c1cccnc1-c1ccncn1)N1CCC(F)(Cc2ccc(C(F)(F)F)nn2)CC1 ; O=C(c1cccnc1-c1ccncc1)N1CCC(F)(Cc2ccc(C(F)(F)F)nn2)CC1] 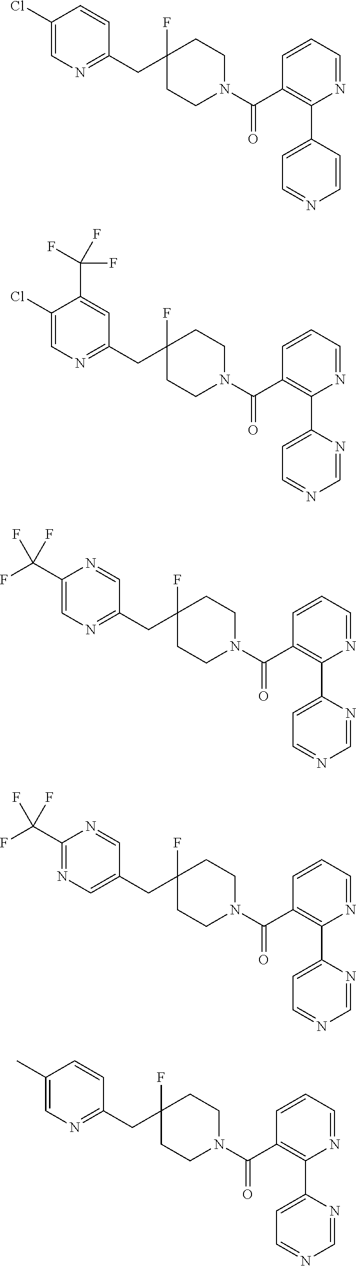 [SMILES: O=C(c1cccnc1-c1ccncc1)N1CCC(F)(Cc2ccc(Cl)cn2)CC1 ; O=C(c1cccnc1-c1ccncn1)N1CCC(F)(Cc2cc(C(F)(F)F)c(Cl)cn2)CC1 ; O=C(c1cccnc1-c1ccncn1)N1CCC(F)(Cc2cnc(C(F)(F)F)cn2)CC1 ; O=C(c1cccnc1-c1ccncn1)N1CCC(F)(Cc2cnc(C(F)(F)F)nc2)CC1 ; Cc1ccc(CC2(F)CCN(C(=O)c3cccnc3-c3ccncn3)CC2)nc1] 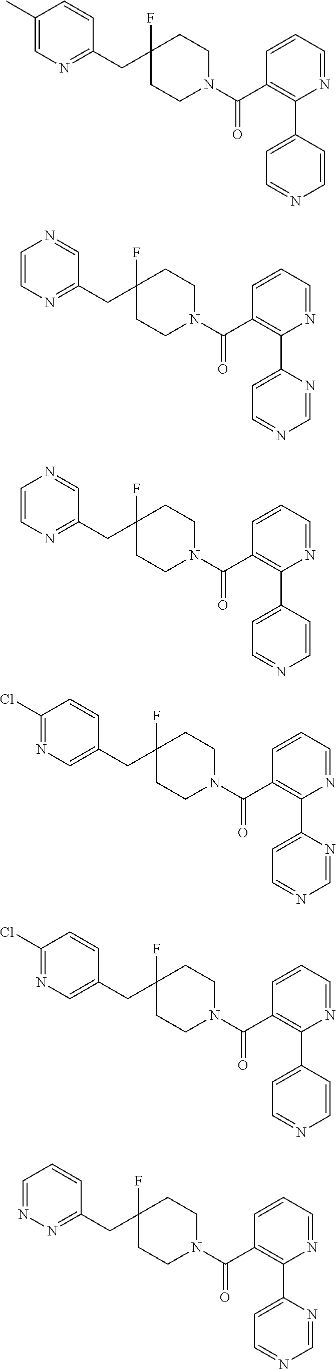 [SMILES: Cc1ccc(CC2(F)CCN(C(=O)c3cccnc3-c3ccncc3)CC2)nc1 ; O=C(c1cccnc1-c1ccncn1)N1CCC(F)(Cc2cnccn2)CC1 ; O=C(c1cccnc1-c1ccncc1)N1CCC(F)(Cc2cnccn2)CC1 ; O=C(c1cccnc1-c1ccncn1)N1CCC(F)(Cc2ccc(Cl)nc2)CC1 ; O=C(c1cccnc1-c1ccncc1)N1CCC(F)(Cc2ccc(Cl)nc2)CC1 ; O=C(c1cccnc1-c1ccncn1)N1CCC(F)(Cc2cccnn2)CC1] 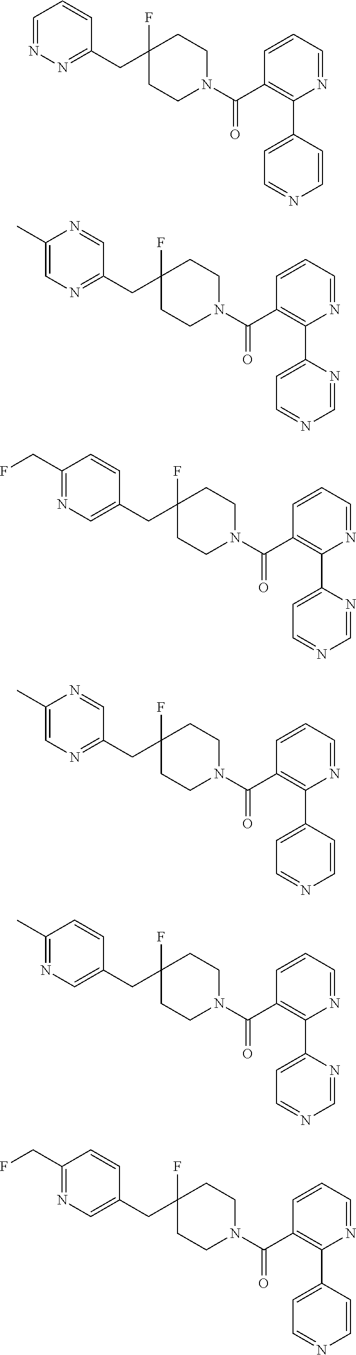 [SMILES: O=C(c1cccnc1-c1ccncc1)N1CCC(F)(Cc2cccnn2)CC1 ; Cc1cnc(CC2(F)CCN(C(=O)c3cccnc3-c3ccncn3)CC2)cn1 ; O=C(c1cccnc1-c1ccncn1)N1CCC(F)(Cc2ccc(CF)nc2)CC1 ; Cc1cnc(CC2(F)CCN(C(=O)c3cccnc3-c3ccncc3)CC2)cn1 ; Cc1ccc(CC2(F)CCN(C(=O)c3cccnc3-c3ccncn3)CC2)cn1 ; O=C(c1cccnc1-c1ccncc1)N1CCC(F)(Cc2ccc(CF)nc2)CC1] 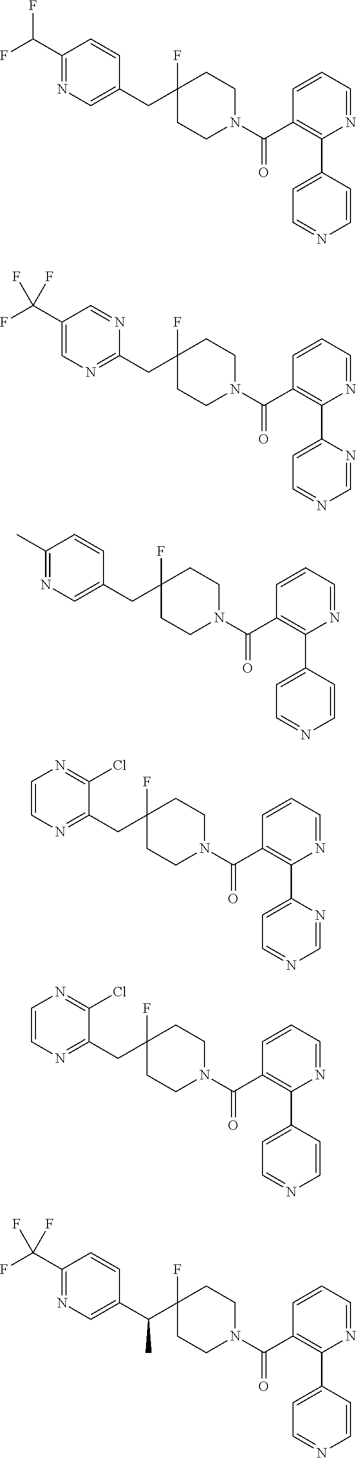 [SMILES: O=C(c1cccnc1-c1ccncc1)N1CCC(F)(Cc2ccc(C(F)F)nc2)CC1 ; O=C(c1cccnc1-c1ccncn1)N1CCC(F)(Cc2ncc(C(F)(F)F)cn2)CC1 ; Cc1ccc(CC2(F)CCN(C(=O)c3cccnc3-c3ccncc3)CC2)cn1 ; O=C(c1cccnc1-c1ccncn1)N1CCC(F)(Cc2nccnc2Cl)CC1 ; O=C(c1cccnc1-c1ccncc1)N1CCC(F)(Cc2nccnc2Cl)CC1 ; C[C@@H](c1ccc(C(F)(F)F)nc1)C1(F)CCN(C(=O)c2cccnc2-c2ccncc2)CC1] 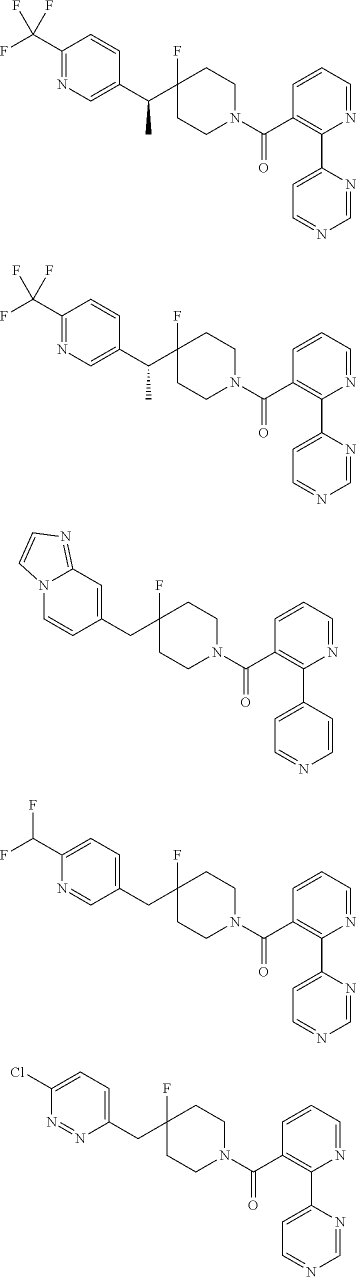 [SMILES: C[C@@H](c1ccc(C(F)(F)F)nc1)C1(F)CCN(C(=O)c2cccnc2-c2ccncn2)CC1 ; C[C@H](c1ccc(C(F)(F)F)nc1)C1(F)CCN(C(=O)c2cccnc2-c2ccncn2)CC1 ; O=C(c1cccnc1-c1ccncc1)N1CCC(F)(Cc2ccn3ccnc3c2)CC1 ; O=C(c1cccnc1-c1ccncn1)N1CCC(F)(Cc2ccc(C(F)F)nc2)CC1 ; O=C(c1cccnc1-c1ccncn1)N1CCC(F)(Cc2ccc(Cl)nn2)CC1] 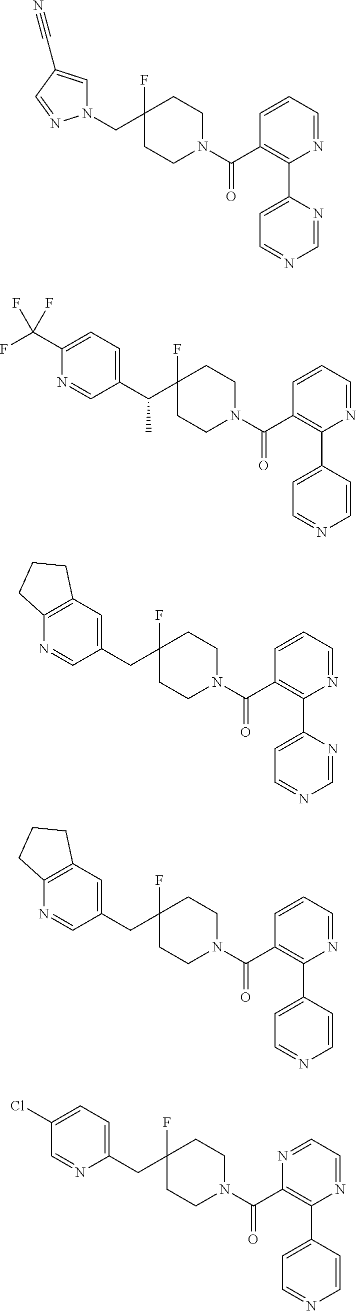 [SMILES: N#Cc1cnn(CC2(F)CCN(C(=O)c3cccnc3-c3ccncn3)CC2)c1 ; C[C@H](c1ccc(C(F)(F)F)nc1)C1(F)CCN(C(=O)c2cccnc2-c2ccncc2)CC1 ; O=C(c1cccnc1-c1ccncn1)N1CCC(F)(Cc2cnc3c(c2)CCC3)CC1 ; O=C(c1cccnc1-c1ccncc1)N1CCC(F)(Cc2cnc3c(c2)CCC3)CC1 ; O=C(c1nccnc1-c1ccncc1)N1CCC(F)(Cc2ccc(Cl)cn2)CC1] 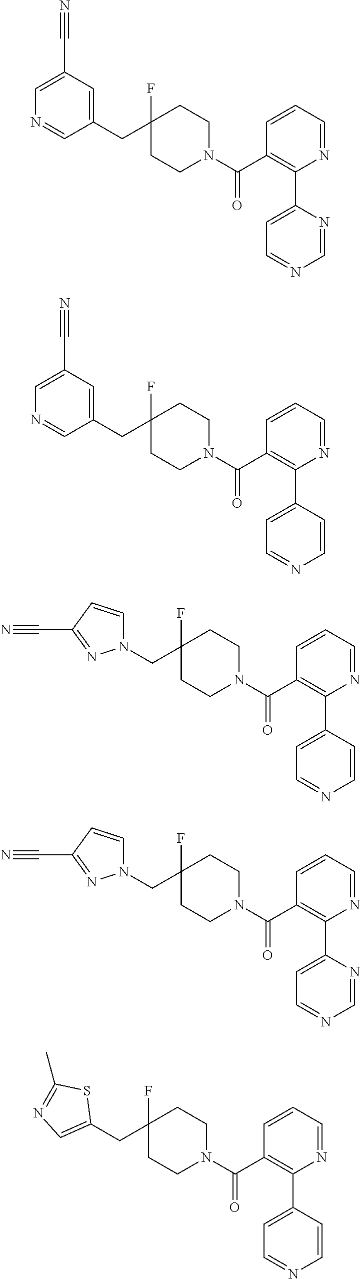 [SMILES: N#Cc1cncc(CC2(F)CCN(C(=O)c3cccnc3-c3ccncn3)CC2)c1 ; N#Cc1cncc(CC2(F)CCN(C(=O)c3cccnc3-c3ccncc3)CC2)c1 ; N#Cc1ccn(CC2(F)CCN(C(=O)c3cccnc3-c3ccncc3)CC2)n1 ; N#Cc1ccn(CC2(F)CCN(C(=O)c3cccnc3-c3ccncn3)CC2)n1 ; Cc1ncc(CC2(F)CCN(C(=O)c3cccnc3-c3ccncc3)CC2)s1] 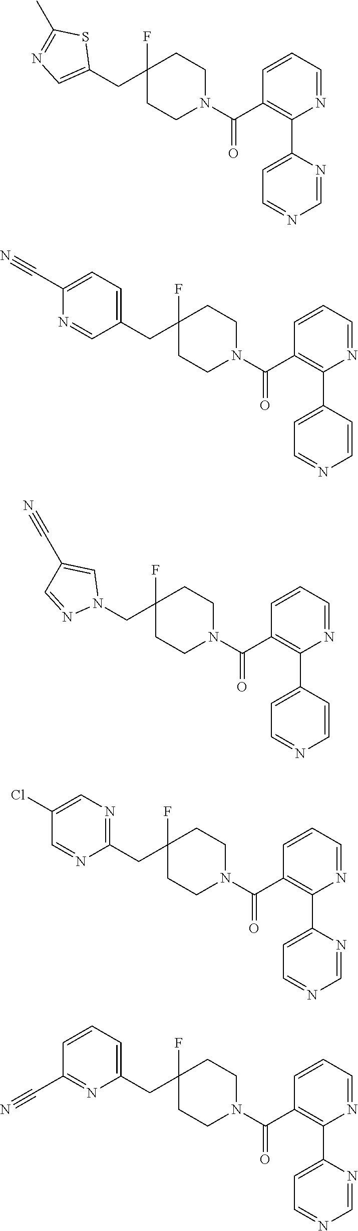 [SMILES: Cc1ncc(CC2(F)CCN(C(=O)c3cccnc3-c3ccncn3)CC2)s1 ; N#Cc1ccc(CC2(F)CCN(C(=O)c3cccnc3-c3ccncc3)CC2)cn1 ; N#Cc1cnn(CC2(F)CCN(C(=O)c3cccnc3-c3ccncc3)CC2)c1 ; O=C(c1cccnc1-c1ccncn1)N1CCC(F)(Cc2ncc(Cl)cn2)CC1 ; N#Cc1cccc(CC2(F)CCN(C(=O)c3cccnc3-c3ccncn3)CC2)n1] 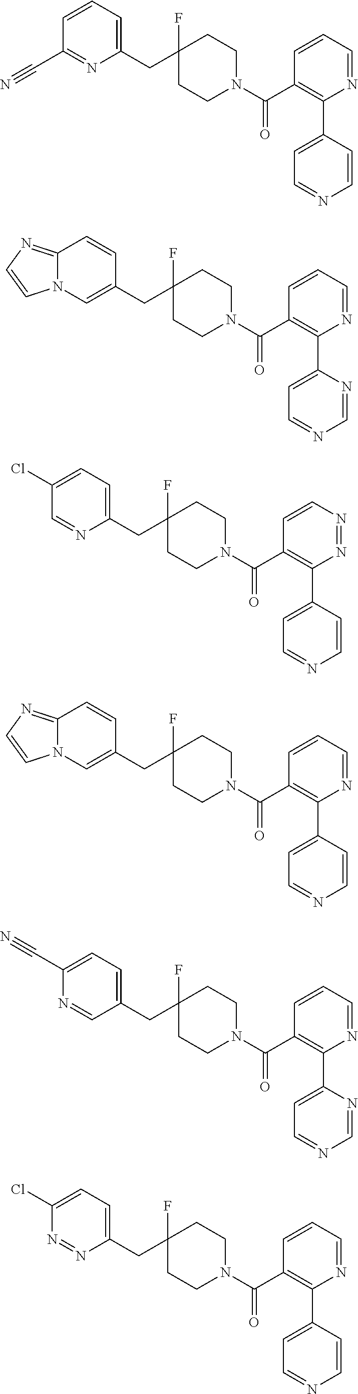 [SMILES: N#Cc1cccc(CC2(F)CCN(C(=O)c3cccnc3-c3ccncc3)CC2)n1 ; O=C(c1cccnc1-c1ccncn1)N1CCC(F)(Cc2ccc3nccn3c2)CC1 ; O=C(c1ccnnc1-c1ccncc1)N1CCC(F)(Cc2ccc(Cl)cn2)CC1 ; O=C(c1cccnc1-c1ccncc1)N1CCC(F)(Cc2ccc3nccn3c2)CC1 ; N#Cc1ccc(CC2(F)CCN(C(=O)c3cccnc3-c3ccncn3)CC2)cn1 ; O=C(c1cccnc1-c1ccncc1)N1CCC(F)(Cc2ccc(Cl)nn2)CC1] 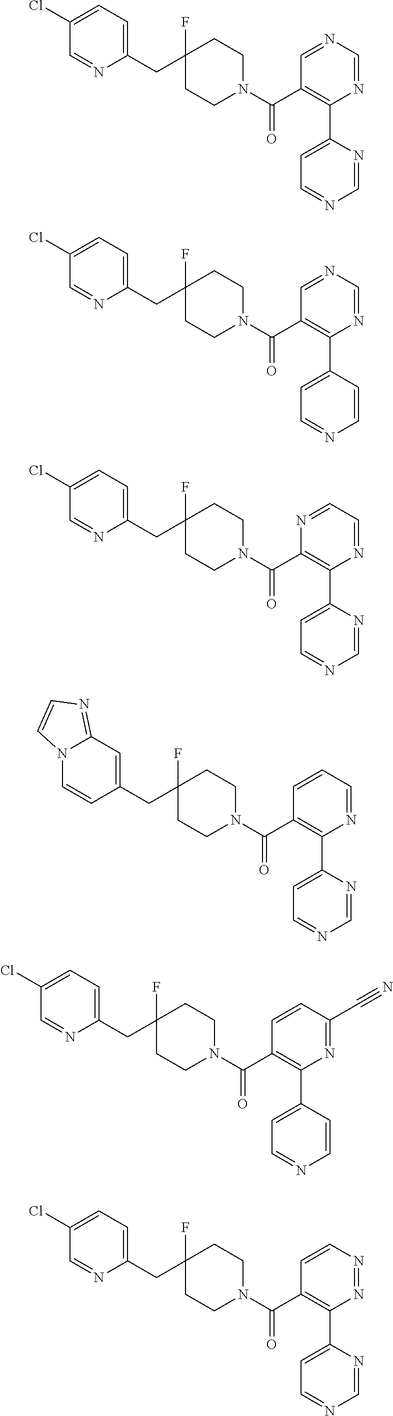 [SMILES: O=C(c1cncnc1-c1ccncn1)N1CCC(F)(Cc2ccc(Cl)cn2)CC1 ; O=C(c1cncnc1-c1ccncc1)N1CCC(F)(Cc2ccc(Cl)cn2)CC1 ; O=C(c1nccnc1-c1ccncn1)N1CCC(F)(Cc2ccc(Cl)cn2)CC1 ; O=C(c1cccnc1-c1ccncn1)N1CCC(F)(Cc2ccn3ccnc3c2)CC1 ; N#Cc1ccc(C(=O)N2CCC(F)(Cc3ccc(Cl)cn3)CC2)c(-c2ccncc2)n1 ; O=C(c1ccnnc1-c1ccncn1)N1CCC(F)(Cc2ccc(Cl)cn2)CC1] 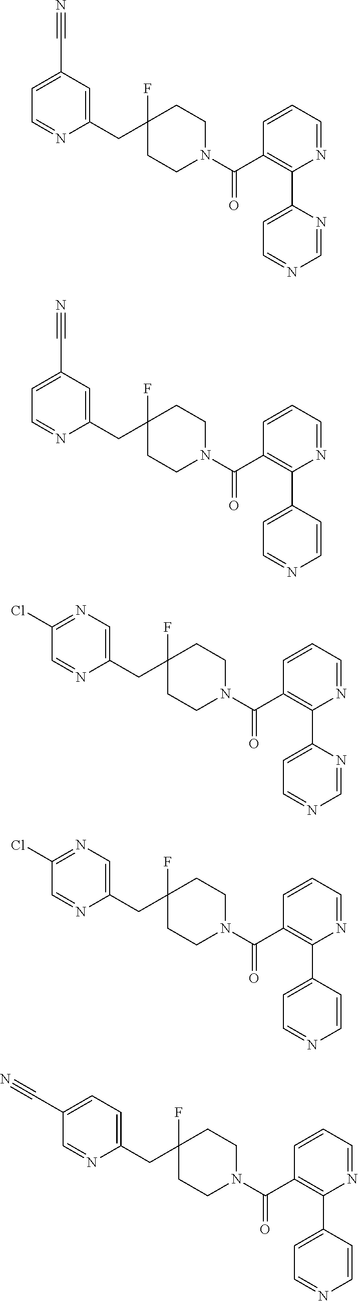 [SMILES: N#Cc1ccnc(CC2(F)CCN(C(=O)c3cccnc3-c3ccncn3)CC2)c1 ; N#Cc1ccnc(CC2(F)CCN(C(=O)c3cccnc3-c3ccncc3)CC2)c1 ; O=C(c1cccnc1-c1ccncn1)N1CCC(F)(Cc2cnc(Cl)cn2)CC1 ; O=C(c1cccnc1-c1ccncc1)N1CCC(F)(Cc2cnc(Cl)cn2)CC1 ; N#Cc1ccc(CC2(F)CCN(C(=O)c3cccnc3-c3ccncc3)CC2)nc1] 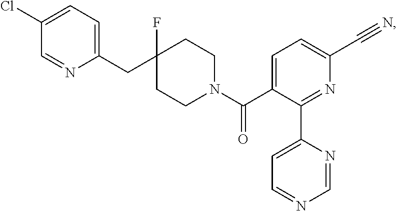 [SMILES: N#Cc1ccc(C(=O)N2CCC(F)(Cc3ccc(Cl)cn3)CC2)c(-c2ccncn2)n1]
or a pharmaceutically acceptable salt thereof.

85. The compound of claim 84, wherein the compound is selected from the group consisting of: 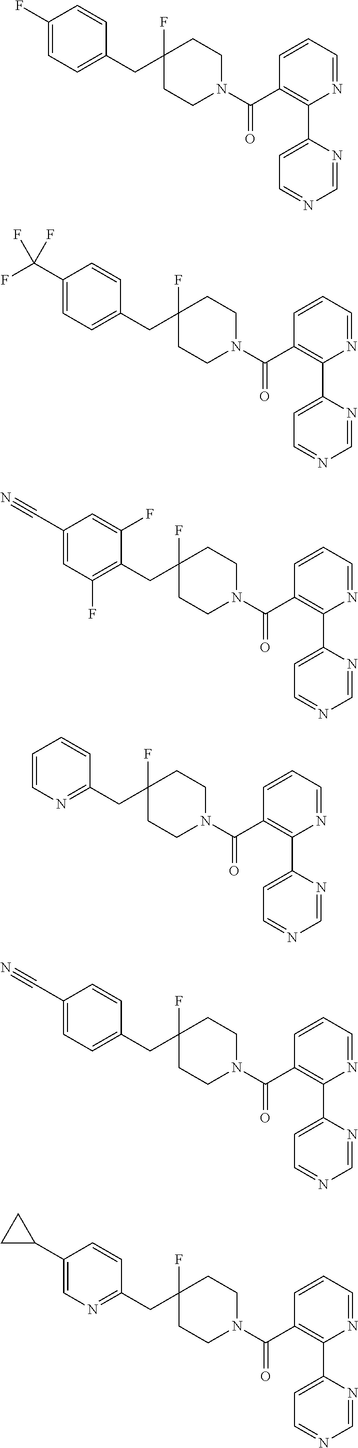 [SMILES: O=C(c1cccnc1-c1ccncn1)N1CCC(F)(Cc2ccc(F)cc2)CC1 ; O=C(c1cccnc1-c1ccncn1)N1CCC(F)(Cc2ccc(C(F)(F)F)cc2)CC1 ; N#Cc1cc(F)c(CC2(F)CCN(C(=O)c3cccnc3-c3ccncn3)CC2)c(F)c1 ; O=C(c1cccnc1-c1ccncn1)N1CCC(F)(Cc2ccccn2)CC1 ; N#Cc1ccc(CC2(F)CCN(C(=O)c3cccnc3-c3ccncn3)CC2)cc1 ; O=C(c1cccnc1-c1ccncn1)N1CCC(F)(Cc2ccc(C3CC3)cn2)CC1] 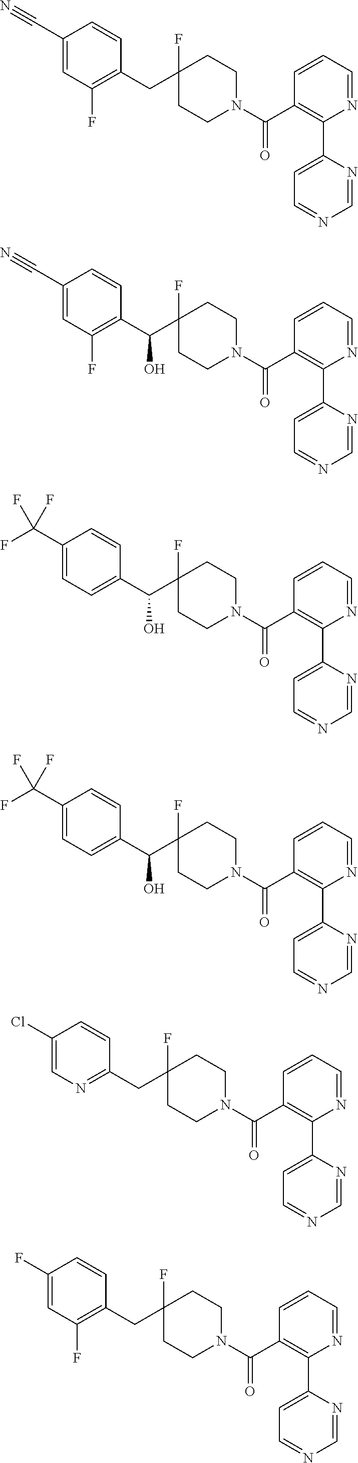 [SMILES: N#Cc1ccc(CC2(F)CCN(C(=O)c3cccnc3-c3ccncn3)CC2)c(F)c1 ; N#Cc1ccc([C@H](O)C2(F)CCN(C(=O)c3cccnc3-c3ccncn3)CC2)c(F)c1 ; O=C(c1cccnc1-c1ccncn1)N1CCC(F)([C@H](O)c2ccc(C(F)(F)F)cc2)CC1 ; O=C(c1cccnc1-c1ccncn1)N1CCC(F)([C@@H](O)c2ccc(C(F)(F)F)cc2)CC1 ; O=C(c1cccnc1-c1ccncn1)N1CCC(F)(Cc2ccc(Cl)cn2)CC1 ; O=C(c1cccnc1-c1ccncn1)N1CCC(F)(Cc2ccc(F)cc2F)CC1] 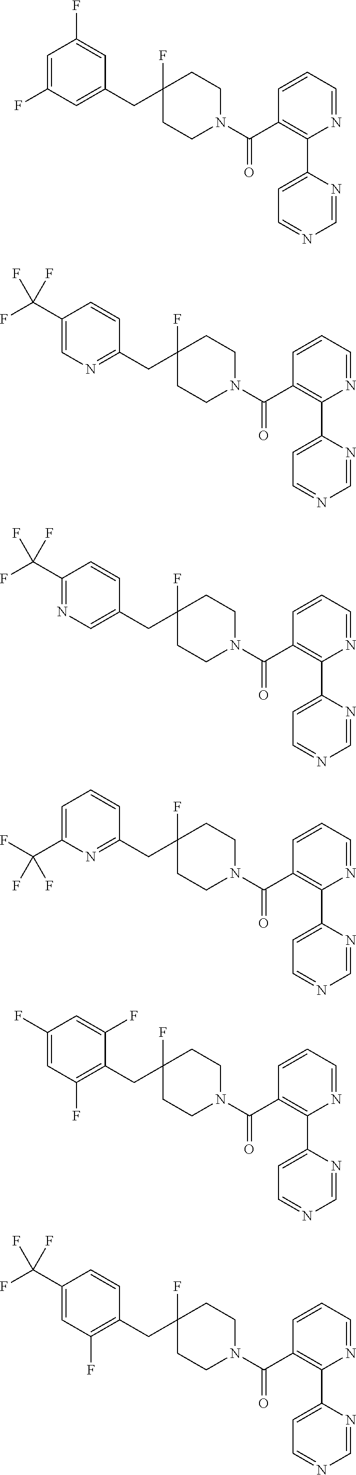 [SMILES: O=C(c1cccnc1-c1ccncn1)N1CCC(F)(Cc2cc(F)cc(F)c2)CC1 ; O=C(c1cccnc1-c1ccncn1)N1CCC(F)(Cc2ccc(C(F)(F)F)cn2)CC1 ; O=C(c1cccnc1-c1ccncn1)N1CCC(F)(Cc2ccc(C(F)(F)F)nc2)CC1 ; O=C(c1cccnc1-c1ccncn1)N1CCC(F)(Cc2cccc(C(F)(F)F)n2)CC1 ; O=C(c1cccnc1-c1ccncn1)N1CCC(F)(Cc2c(F)cc(F)cc2F)CC1 ; O=C(c1cccnc1-c1ccncn1)N1CCC(F)(Cc2ccc(C(F)(F)F)cc2F)CC1] 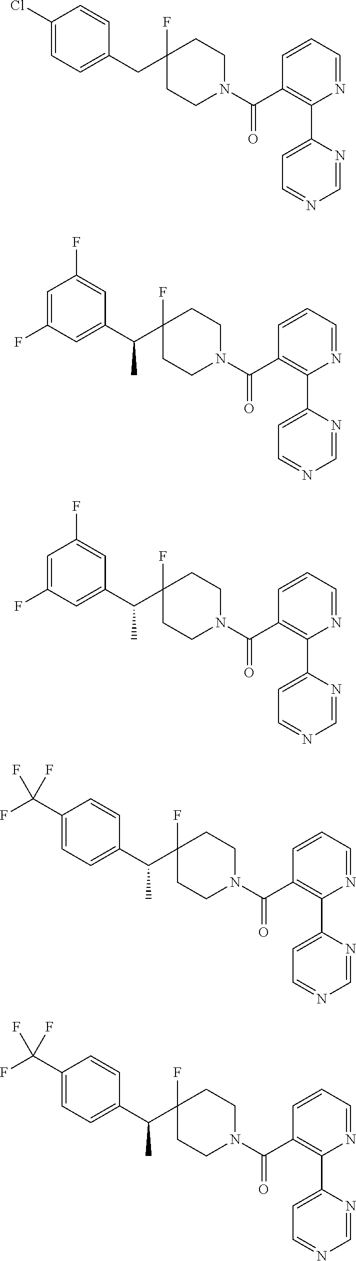 [SMILES: O=C(c1cccnc1-c1ccncn1)N1CCC(F)(Cc2ccc(Cl)cc2)CC1 ; C[C@@H](c1cc(F)cc(F)c1)C1(F)CCN(C(=O)c2cccnc2-c2ccncn2)CC1 ; C[C@H](c1cc(F)cc(F)c1)C1(F)CCN(C(=O)c2cccnc2-c2ccncn2)CC1 ; C[C@H](c1ccc(C(F)(F)F)cc1)C1(F)CCN(C(=O)c2cccnc2-c2ccncn2)CC1 ; C[C@@H](c1ccc(C(F)(F)F)cc1)C1(F)CCN(C(=O)c2cccnc2-c2ccncn2)CC1] 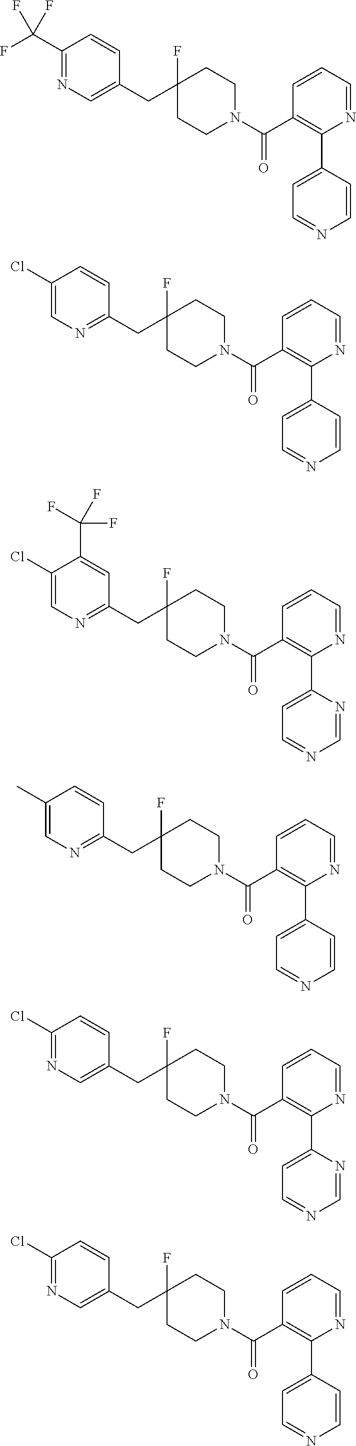 [SMILES: O=C(c1cccnc1-c1ccncc1)N1CCC(F)(Cc2ccc(C(F)(F)F)nc2)CC1 ; O=C(c1cccnc1-c1ccncc1)N1CCC(F)(Cc2ccc(Cl)cn2)CC1 ; O=C(c1cccnc1-c1ccncn1)N1CCC(F)(Cc2cc(C(F)(F)F)c(Cl)cn2)CC1 ; Cc1ccc(CC2(F)CCN(C(=O)c3cccnc3-c3ccncc3)CC2)nc1 ; O=C(c1cccnc1-c1ccncn1)N1CCC(F)(Cc2ccc(Cl)nc2)CC1 ; O=C(c1cccnc1-c1ccncc1)N1CCC(F)(Cc2ccc(Cl)nc2)CC1] 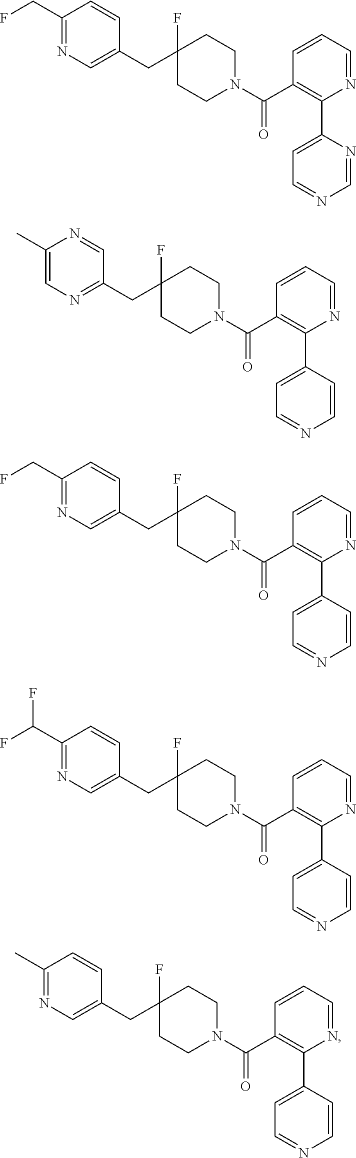 [SMILES: O=C(c1cccnc1-c1ccncn1)N1CCC(F)(Cc2ccc(CF)nc2)CC1 ; Cc1cnc(CC2(F)CCN(C(=O)c3cccnc3-c3ccncc3)CC2)cn1 ; O=C(c1cccnc1-c1ccncc1)N1CCC(F)(Cc2ccc(CF)nc2)CC1 ; O=C(c1cccnc1-c1ccncc1)N1CCC(F)(Cc2ccc(C(F)F)nc2)CC1 ; Cc1ccc(CC2(F)CCN(C(=O)c3cccnc3-c3ccncc3)CC2)cn1]
or a pharmaceutically acceptable salt thereof.

86. The compound of claim 84, wherein the compound is selected from the group consisting of: 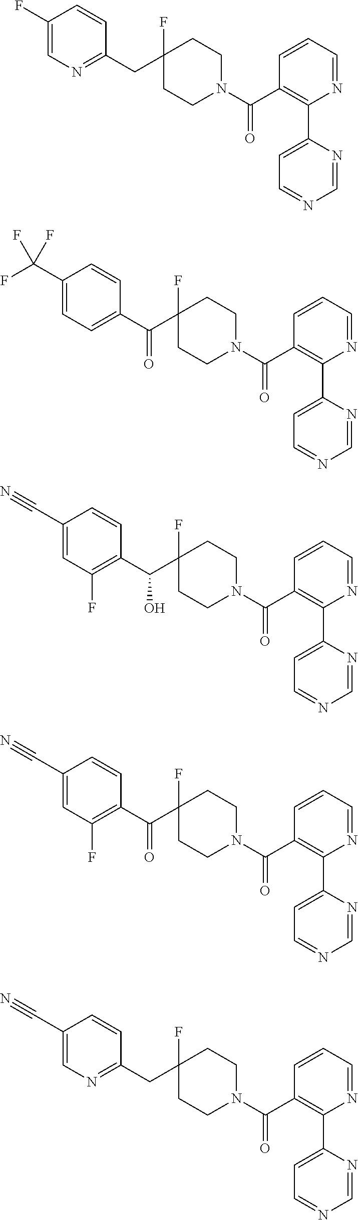 [SMILES: O=C(c1cccnc1-c1ccncn1)N1CCC(F)(Cc2ccc(F)cn2)CC1 ; O=C(c1cccnc1-c1ccncn1)N1CCC(F)(C(=O)c2ccc(C(F)(F)F)cc2)CC1 ; N#Cc1ccc([C@@H](O)C2(F)CCN(C(=O)c3cccnc3-c3ccncn3)CC2)c(F)c1 ; N#Cc1ccc(C(=O)C2(F)CCN(C(=O)c3cccnc3-c3ccncn3)CC2)c(F)c1 ; N#Cc1ccc(CC2(F)CCN(C(=O)c3cccnc3-c3ccncn3)CC2)nc1] 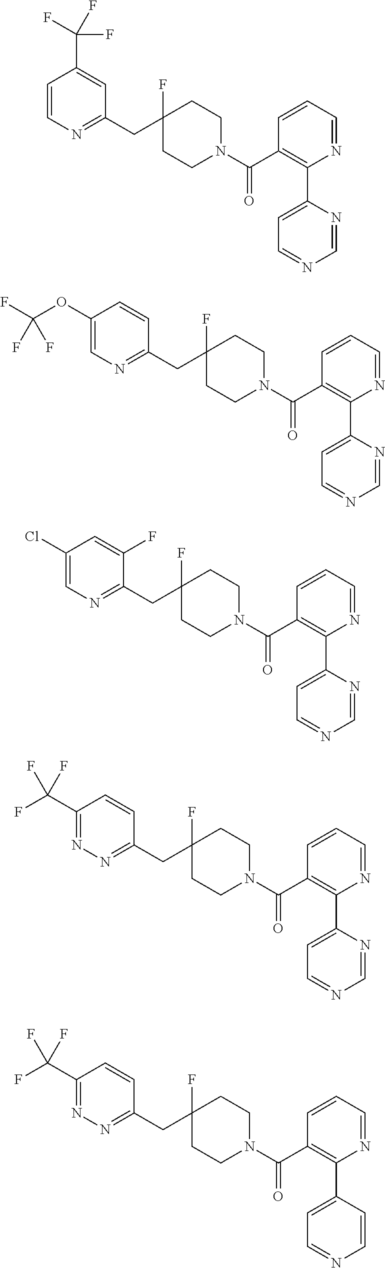 [SMILES: O=C(c1cccnc1-c1ccncn1)N1CCC(F)(Cc2cc(C(F)(F)F)ccn2)CC1 ; O=C(c1cccnc1-c1ccncn1)N1CCC(F)(Cc2ccc(OC(F)(F)F)cn2)CC1 ; O=C(c1cccnc1-c1ccncn1)N1CCC(F)(Cc2ncc(Cl)cc2F)CC1 ; O=C(c1cccnc1-c1ccncn1)N1CCC(F)(Cc2ccc(C(F)(F)F)nn2)CC1 ; O=C(c1cccnc1-c1ccncc1)N1CCC(F)(Cc2ccc(C(F)(F)F)nn2)CC1] 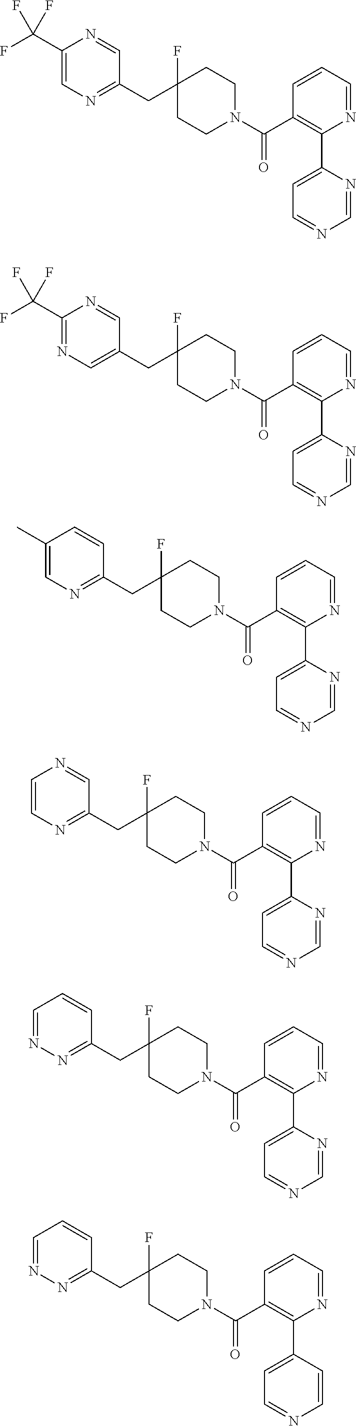 [SMILES: O=C(c1cccnc1-c1ccncn1)N1CCC(F)(Cc2cnc(C(F)(F)F)cn2)CC1 ; O=C(c1cccnc1-c1ccncn1)N1CCC(F)(Cc2cnc(C(F)(F)F)nc2)CC1 ; Cc1ccc(CC2(F)CCN(C(=O)c3cccnc3-c3ccncn3)CC2)nc1 ; O=C(c1cccnc1-c1ccncn1)N1CCC(F)(Cc2cnccn2)CC1 ; O=C(c1cccnc1-c1ccncn1)N1CCC(F)(Cc2cccnn2)CC1 ; O=C(c1cccnc1-c1ccncc1)N1CCC(F)(Cc2cccnn2)CC1] 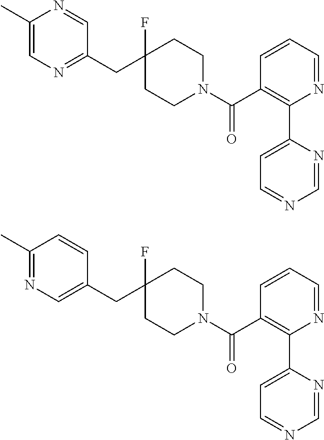 [SMILES: Cc1cnc(CC2(F)CCN(C(=O)c3cccnc3-c3ccncn3)CC2)cn1 ; Cc1ccc(CC2(F)CCN(C(=O)c3cccnc3-c3ccncn3)CC2)cn1]
or a pharmaceutically acceptable salt thereof.

87. A pharmaceutical composition comprising a compound of any one of claims 1-86, or a pharmaceutically acceptable salt thereof, and a pharmaceutically acceptable carrier.

88. A method for treating or preventing a disease or disorder involving the inhibition of CYP46A1 in a subject in need thereof, comprising administering to the subject therapeutically effective amount of a compound of any one of claims 1-86, or a pharmaceutically acceptable salt thereof, or a pharmaceutical composition of claim 87, wherein the disease or disorder involving the inhibition of CYP46A1 is selected from the group consisting of a neurodegenerative disorder, epilepsy, developmental and epileptic encephalopathies, psychiatric disorders, and spasms.

89. The method of claim 88, wherein the disease or disorder involving the inhibition of CYP46A1 is a neurodegenerative disorder.

90. The method of claim 89, wherein the neurodegenerative disorder is selected from the group consisting of Alzheimer's disease, mild cognitive impairment, Huntington's disease, Parkinson's disease, amyotrophic lateral sclerosis, traumatic brain injury, cerebral infarction, glaucoma, and multiple sclerosis.

91. The method of claim 88, wherein the disease or disorder involving the inhibition of CYP46A1 is epilepsy.

92. The method of claim 88, wherein the disease or disorder involving the inhibition of CYP46A1 is developmental and epileptic encephalopathies.

93. The method of claim 88, wherein the disease or disorder involving the inhibition of CYP46A1 is a psychiatric disorder.

94. The method of claim 93, wherein the psychiatric disorder is selected from the group consisting of schizophrenia, autism spectrum disorder, delusional disorder, schizoaffective disorder, and depression.

95. The method of claim 88, wherein the disease or disorder involving the inhibition of CYP46A1 is spasms.

96. A compound or pharmaceutically acceptable salt thereof of any one of claims 1-86, or a pharmaceutically acceptable salt thereof, or a pharmaceutical composition of claim 87, for use in treating or preventing a disease or disorder involving the inhibition of CYP46A1 in a subject, wherein the disease or disorder involving the inhibition of CYP46A1 is selected from the group consisting of a neurodegenerative disorder, epilepsy, developmental and epileptic encephalopathies, psychiatric disorders, and spasms.

97. The compound or pharmaceutically acceptable salt thereof or pharmaceutical composition for use of claim 96, wherein the disease or disorder involving the inhibition of CYP46A1 is a neurodegenerative disorder.

98. The compound or pharmaceutically acceptable salt thereof or pharmaceutical composition for use of claim 97, wherein the neurodegenerative disorder is selected from the group consisting of Alzheimer's disease, mild cognitive impairment, Huntington's disease, Parkinson's disease, amyotrophic lateral sclerosis, traumatic brain injury, cerebral infarction, glaucoma, and multiple sclerosis.

99. The compound or pharmaceutically acceptable salt thereof or pharmaceutical composition for use of claim 96, wherein the disease or disorder involving the inhibition of CYP46A1 is epilepsy.

100. The compound or pharmaceutically acceptable salt thereof or pharmaceutical composition for use of claim 96, wherein the disease or disorder involving the inhibition of CYP46A1 is developmental and epileptic encephalopathies.

101. The compound or pharmaceutically acceptable salt thereof or pharmaceutical composition for use of claim 96, wherein the disease or disorder involving the inhibition of CYP46A1 is a psychiatric disorder.

102. The compound or pharmaceutically acceptable salt thereof or pharmaceutical composition for use of claim 101, wherein the psychiatric disorder is selected from the group consisting of schizophrenia, autism spectrum disorder, delusional disorder, schizoaffective disorder, and depression.

103. The compound or pharmaceutically acceptable salt thereof or pharmaceutical composition for use of claim 96, wherein the disease or disorder involving the inhibition of CYP46A1 is spasms.

104. Use of a compound or pharmaceutically acceptable salt thereof according to any one of claims 1-86, or a pharmaceutical composition of claim 87, in the manufacture of a medicament for treating or preventing a disease or disorder involving the inhibition of CYP46A1 in a subject, wherein the disease or disorder involving the inhibition of CYP46A1 is selected from the group consisting of a neurodegenerative disorder, epilepsy, developmental and epileptic encephalopathies, psychiatric disorders, and spasms.

105. The use of claim 104, wherein the disease or disorder involving the inhibition of CYP46A1 is a neurodegenerative disorder.

106. The use of claim 105, wherein the neurodegenerative disorder is selected from the group consisting of Alzheimer's disease, mild cognitive impairment, Huntington's disease, Parkinson's disease, amyotrophic lateral sclerosis, traumatic brain injury, cerebral infarction, glaucoma, and multiple sclerosis.

107. The use of claim 104, wherein the disease or disorder involving the inhibition of CYP46A1 is epilepsy.

108. The use of claim 104, wherein the disease or disorder involving the inhibition of CYP46A1 is developmental and epileptic encephalopathies.

109. The use of claim 104, wherein the disease or disorder involving the inhibition of CYP46A1 is a psychiatric disorder.

110. The use of claim 109, wherein the psychiatric disorder is selected from the group consisting of schizophrenia, autism spectrum disorder, delusional disorder, schizoaffective disorder, and depression.

111. The use of claim 104, wherein the disease or disorder involving the inhibition of CYP46A1 is spasms.
